(19) United States
(12) Patent Application Publication
(10) Pub. No.: US 2025/0273287 A1
(43) Pub. Date: Aug. 28, 2025

(54) GLYCOSYLATION ENGINEERING

(71) Applicant: The Regents of the University of California, Oakland, CA (US)

(72) Inventors: Nathan Lewis, San Diego, CA (US); Benjamin Kellman, Beaverton, OR (US); Daniel Sandoval, San Diego, CA (US); Daniela Nachmanson, San Diego, CA (US); Wan-tien Chiang, San Diego, CA (US)

(21) Appl. No.: 18/858,181

(22) PCT Filed: Apr. 18, 2023
(86) PCT No.: PCT/US2023/065895

§ 371 Date: Oct. 18, 2024

Related U.S. Application Data
(60) Provisional application No. 63/332,385, filed on Apr. 19, 2022.

(51) Int. Cl. G16B 15/20; C07K 14/005

(52) U.S. Cl. CPC G16B 15/20; C07K 14/005; C12N 2740/16122; C12N 2770/20022

(57) ABSTRACT

Disclosed herein are methods and systems for engineering glycosylation. The methods and systems may use structure of sequence information of biomolecules to predict glycosylation features. The methods and systems may employ one or more trained algorithms described herein.

### CROSS REFERENCE TO RELATED APPLICATIONS

This application claims priority to U.S. Provisional Patent Application No. 63/332,385 filed on Apr. 19, 2022, the entire contents of which are incorporated herein by reference.

### STATEMENT AS TO FEDERALLY SPONSORED RESEARCH

This invention was made with government support under Contract number GM119850 awarded by the National Institutes of Health. The government has certain rights in the invention.

### BACKGROUND OF THE INVENTION

Glycosylation is the reaction in which a carbohydrate (or ‘glycan’), i.e. a glycosyl donor, is attached to a hydroxyl or other functional group of another molecule (a glycosyl acceptor) in order to form a glycoconjugate. Such glycoconjugates serve various chemical and biological functions. Unlike biosynthesis of other biological molecules, glycan synthesis and glycosylation have resisted characterization as templated processes which has led to poor understanding of the structural and sequence factors that impact them.

### SUMMARY OF THE INVENTION

Recognized herein is a need for systems and methods that lead to a predictive understanding of glycosylation and glycan biosynthesis. Using curated datasets of glycosylation features associated with protein structures and sequences, associations between these biomolecular features and glycosylation features may be determined. In some embodiments, such associations provide insights into the principles underlying glycan biosynthesis as well as contribute to methods and systems for engineering novel glycosylated biomolecules.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. The present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates discovered mechanisms of glycan biosynthesis considered in comparison to previously known templated biosynthesis of DNA, RNA, and protein.

FIG. 2A shows feature weights from a Factor Analysis with Mixed Data (FAMD) (of annotated sites. FIG. 2B shows a two-dimensional projection of the FAMD using a Uniform Manifold Approximation and Projection (UMAP). Each point is a site on a glycoprotein, each color indicates the source of that protein. FIG. 2C shows an FDR distribution of p-values from a multivariate gaussian trained on the UMAP 2D projection.

FIG. 3A illustrates a specification of the event space to each observed glycosylation event at a glycosylation site. FIG. 3B shows a volcano plot of the log odds ratio and False Discovery Rate (FDR) adjusted p-values from a Fisher exact test between glycan and protein structure occurrence. Proximal AAs for the top 20 most significant relations are shown. FIG. 3C shows a Kullback-Leibler divergence when either glycan structures, G, or protein structures, P, are specified as present, 1, or absent, 0. FIG. 3D shows probabilities of glycan structures when protein structures were known or “fixed.” FIG. 3E shows protein structure probabilities conditioned on fixed glycan structures. FIG. 3F illustrates non-independence, absolute difference between conditional and marginal probabilities (Pr(A|B)−Pr(A)), stratified by glycan motif size for protein-glycan relations when glycan structures are fixed and when protein structures are fixed.

FIGS. 4A-4C show distributions of high-confidence/high-certainty (OR-derived probability is effectively 1 or 0; within 0.001) amino acid-glycan IMRs. Each plot is split indicating when the protein structure is fixed present (T, left) or absent (F, right). FIG. 4A shows distribution of high-confidence (grey) and other (black) aa-glycan IMRs out of 60,000 IMRs. FIG. 4B shows a number of unique glycan substructures involved in high-confidence aa-glycan IMRs. FIG. 4C shows high-certainty aa-glycan IMRs by amino-acid (x-axis) proximity type and probability: close to zero (top) or close to one (bottom).

FIG. 5A shows the number of significant (FDR<0.1, |log(OR)|>0.1) IMRs relating to structurally proximal AAs (N+6 Å), sequence proximal AAs C-terminal (N+5), N-terminal (N−5), or either direction (N+/−5), predicted secondary structure from sequence (SSpro8) and structure (DSSP): alpha-helix (ss.H), extended strand (ss.E), beta-bridge (ss.B), turn (ss.T) bend (ss.S), other (ss.C). FIG. 5B show The Odds Ratios (x-axis) and FDR (y-axis) for IMRs relating glycan motifs to structurally (DSSP) estimated Turns (T). FIGS. 5C and 5D show IMRs (FDR<0.1, |log(OR)|>0.1) relating structurally proximal amino acids to motifs stratified by the number of Sialic Acids (FIG. 5C) and 4-Sulfated GalNAc (FIG. 5D). FIG. 5E shows the Spearman correlation between the monosaccharide count of glycan substructures from protein-structure features; protein structure features with an average absolute correlation >0.2 were retained. FIGS. 5F and 5G show IMRs (FDR<0.1, log(OR)|>0.1) compared across two sequence-proximal (FIG. 5F) and structure-proximal (FIG. 5G) amino acids, phenylalanine (F) and tryptophan (W). The direct comparison of proximal-amino acid effects visualized the expected change in glycosylation associated with that substitution; this expected change is the basic concept underlying “glycoimpact” (the expected impact on glycosylation of a protein-structure change and/or amino-acid substitution. FIG. 5H illustrates a network depicting the predicted magnitude of glycoimpact (edge color) of structure-proximal (within 5 Å) substitutions for low impact (Blosum64) substitutions. Predicted glycoimpact is calculated as Euclidean distance between the significant log(OR) for all glycomotifs associated with a protein structure. FIG. 5H shows glycoimpact predicted from BLAMO 0.5:0.1 (|log(OR)|>0.5, FDR<0.1). The present disclosure uses glycoimpact from BLAMO 0.5:0.1 unless otherwise specified.

FIG. 6 shows for additional thresholds, raw scores, and sequence-proximal substitution predictions. BLAMO 0.1:0.1, BLAMO 0.5:0.1, and BLAMO 1:0.1 from left to right.

FIG. 7A shows a comparison of the error between the PAM and BLOSUM substitution matrices and the glycoimpact for corresponding substitutions. Linear regressions are split into two relevant ranges: null glycoimpact (<2.5) and impactful (>2.5). Glycoimpact scores from BLAMO 0.5:0.1 were used; those computed from strong IMRs (log(OR)|>0.5, FDR<0.1). Error (y-axis) was calculated as the root mean square error (RMSE) between PAM and BLOSUM scores. Multiple versions of the PAM (PAM30-250) and BLOSUM (BLOSUM45-100) were examined; all pairings are shown in FIG. 8. FIG. 7B illustrates null and impactful glycoimpact (BLAMO 0.5:0.1) stratified by pathogenicity in ClinVar of mutations within 20 Å of an N-glycosylation site. FIG. 7C shows a hierarchical biclustered heatmap (average-linkage with Euclidean distance) of Spearman correlations between glycoimpact (BLAMO0.5:0.1) and error between various pathogenicity predictions. Prediction-type and protein structure indicate the training data used to build various pathogenicity prediction tools. FIG. 7D shows the minimum distance from all residues within human PrP to the N197 or N181 glycosylation sites. Residues are stratified by all sites (All) and causative mutations of prion disease including Creutzfeldt-Jakob disease (CJD) and Gerstmann-Straussler disease (GSD). Significance determined by a one-sided Wilcoxon test. For site-proximity to each glycosylation site, see FIG. 9. FIG. 7E illustrates the number of high-ranking (p<L/5 to p<L/3) evolutionary coupling events between glycosites (GN), asparagines (N), or any residue (AA) with all other residues in each of 2,005 alignments. FIG. 7F shows evolutionary coupling (EC) probability between serine or threonine with a glycosite (GN), any asparagine (N), or any amino acid (AA). Serines and threonines considered appear two residues C-terminal to the GN, N or X (N+2). The upper right sub-panel of FIG. 7G shows the proportion of high-ranking ECs (Rank<L) at N+/−i relative to a glycosylation site (GN, highlighted by pointer), asparagine (N) or any amino acid (X). The significance of EC-enrichment with glycosites was measured with a hypergeometric enrichment comparing the proportion of high-ranking ECs with GN vs those with either N, or X at the same relative position (black-lines). The central panel of FIG. 7G shows an aggregation of all residue-glycosite enrichments from at N+/−10. The proportion of high-ranking ECs for each amino acid (rows) at the column-specified relative position was compared with GN, N, and X. An opaque square indicates that for that residue (row) in that position (column), high-EC proportion is higher with GN than N. An opaque triangle indicates high-EC proportion is higher with GN than any amino acid (AA). A transparent triangle or square indicates GN was not significantly more coupled. Significance was assessed at multiple EC-rank thresholds between L/3 and 3L and p-values were pooled using a Fisher's method; FDR was used to correct for multiple testing. FIG. 7H illustrates a hierarchical clustering of coupling-masked (Rank<4 L) amino acids surrounding (+/−6aa) a glycosite. Each of 5 clusters was summarized as a motif. Height is the log of cumulative reciprocal EC-Rank with a pseudo-count of 0.25. The asparagine at the center was fixed at a height of 2 for context. Residues are colored by chemical properties; this analysis is repeated with 25 clusters in FIG. 14. FIG. 7I shows Glycosite Alignments corresponding to GlyConnect-documented tetra-antennary structures (Hex:7 HexNAc:6) with no sialic acid or fucose. Abbreviated alignments are shown above the brown middle-most “conservation” track. Below the conservation track, the first (top line) and second (bottom line) most popular amino acids are displayed for each position N+/−30. The full alignment can be found in FIG. 15. Consensus amino acids consistent with other analyses are highlighted in bold and marked with a “+” indicating glycosite-coupling (FIG. 7G) or “*” indicating IMR-associated (FIG. 5A). FIG. 7I discloses SEQ ID NOS 1-6, respectively, in order of appearance.

FIG. 8 shows the root mean square error between PAM and BLOSUM substitution scores correlated with predicted glycoimpact.

FIG. 9 show the minimum distance between amino acids within human PrP for disease and library mutants (FIGS. 9A and 9B) and low expression mutants (FIGS. 9C and 9D).

FIG. 10 show the correlation between glycoimpact and pathogenicity score pairwise errors when pathogenicity scores are shuffled within score.

FIG. 11A shows GEE-calculated odds ratio (x-axis) and FDR-adjusted p-values defining IMRs from PGES-DB denoting relations between sequence (triangle & square) or structural protein features (circle & plus) and motifs containing >3 mannose (hybrid & high-mannose). FIG. 11B illustrates the range of observed complex glycan to high-mannose/hybrid glycans (N203/N3) at each site on the HIV envelope gp160 (BG505 SOSIP.664, PDB:4TVP). FIG. 11C shows distributions of low/high-complexity (N203/N3) stratified by proximal protein structure features. FIG. 11D shows GEE-learned IMRs relating to Sequence-proximal (upstream/N-terminal) effects of lie and Phe. FIG. 11E shows IgG allotypes, Phe299 (circle) and 1299 (triangle), segregated by Principal Component Analysis of relative abundance across BALB/c and C57BL/6 mice. FIG. 11F shows Galactose (Gal) and Sialylation (Neu5Ac) relative abundance distributions for IgG1 Phe299 and Ile299 allotypes across BALB/c and C57BL/6 mice. FIG. 11G shows the mean proportion of mass-spectroscopy-observed peptide fragments with mass offsets corresponding to Complex, Oligomannose/Hybrid, or unoccupied glycosylation sites in the SARS-CoV-2 spike S1 across the original 2019 strain, and the Delta and Gamma variants.

FIGS. 12A-12F illustrate Protein-Glycan structure relations in HIV ENV gp160. Relationships between glycosylation feature (specifically the high-mannose/hybrid to complex ratio shown in FIGS. 11B-C; y-axis) and specific protein structure features are shown. Structural elements illustrated number of glycosite 3D-proximinal Phe (e.g., within 6 Å of the glycosite) (struct_aa.F; FIG. 12A), number of glyosite-3D proximal Leu (e.g., within 6 Å of the glycosite) (struct_aa.L; FIG. 12B), number of glyosite-3D proximal Gly (e.g., within 6 Å of the glycosite) (struct aa.G; FIG. 12B), number of downstream Cys (e.g., up to 6 amino acids) downstream of the glycosite (seq_aaDown.C; FIG. 12D), number of downstream Asn (e.g., up to 6 amino acids) downstream of the glycosite (seq_aaDown.N; FIG. 12E), and number of downstream Arg (e.g., within 6 Å) downstream of the glycosite (struct_aa.R; FIG. 12F). Where “Mann_v_Complex” indicates the Low/High-complexity ratio (N203/N3) shown in FIGS. 11B and 11C.

FIG. 13 shows IgG3 N-gly cosylation measurements in mice.

FIG. 14 shows EC-masked extended sequon clustering with motif logos describing high-ranking glycosite-coupled ECs at each position. Figure discloses SEQ ID NOS 1, 7-8, 2, 2-3, 9-19, 4, 20, 20, 19, 21-23, 16, 15, 17-18, 24, 23, 25-36, 34, 34, 34, 31, 24, 37, 15, 15, 37-46, 29, 47-49, 15, 37, 9, 3, 50-53, 52-60, 60-61, 9, 3, 55, 54, and 4-6, respectively, in order of appearance.

FIG. 15 illustrates glycosite alignment for tetra-antennary structures with no sialic acid or fucose from Glycositealign at glyconnect.

FIGS. 16A-16B show Fisher Odds Ratios (OR) estimated IMRs indicating the preference for afucosylation (square) or core-fucosylation (circle) across multiple antibody allotypes. IMRs above y=x (dotted line) are correlated with the mutant allotype and/or anticorrelated with the wild-type. Each substitution is written relative to PODOX5 with an N297 glycosite. Plots show IMRs relating to (FIG. 16A) structure-specific IMRs, within 5 Å, and (FIG. 16B) sequence-specific IMRs, within 5 amino acids up or down stream.

FIG. 17A illustrates model architectures to analyze glycan sequences. FIG. 17B illustrates the full model architecture of a trained algorithm as described herein.

FIG. 18A shows the dependence of glycan feature prediction performance on occurrence. Using a trained algorithm as described herein, the averaged prediction performance of GlyCompare features against their counts in a dataset were plotted. FIG. 18B illustrates GlyCompare feature accuracy distribution. A histogram of the prediction performance for all observed GlyCompare features is shown. FIG. 18C shows a t-SNE visualizing the glycan representation learned by InSaNNe for all GlyCompare features. Each feature was shaded by its averaged prediction performance to identify structurally related clusters of glycan features that are more difficult to predict for InSaNNe. Ovals highlight clusters of difficult-to-predict GlyCompare features. FIG. 18D shows a visualization of the prediction performance depending on the sequon. For all sequons in the dataset, prediction performance was averaged over all glycans and sequons were colored by prediction performance to observe whether any sequon clusters would be difficult to predict for InSaNNe. Clusters were labeled as to whether they contained sequons with N-linked or 0-linked glycans. FIG. 18E illustrates a comparison of experimentally observed and predicted glycans at a glycosylation site of human uromodulin. The sequon GTVLTRNETHATYS (SEQ ID NO: 62) was used to predict permissible glycans using the trained InSaNNe model. The top 100 predicted glycans were analyzed as to their characteristics and examples are shown. FIG. 18F illustrates predicting glycans for an HIV-1 Env sequon. Using the sequon PVQINCTRPN (SEQ ID NO: 63) as input for the trained algorithm, a t-SNE of the glycan representations learned the trained algorithm was colored by the predicted probability of occurring on that sequon to identify structurally related glycans that were predicted to be present. FIG. 18G shows comparing predicted glycans at sequons of the HIV-1 Env protein. For three sequons, a t-SNE of the glycan representations learned by the trained algorithm was colored by the predicted probability by the trained algorithm and indicated structural glycan features common to predicted glycans. FIG. 18G discloses SEQ ID NOS 65-67, respectively, in order of appearance.

FIG. 19 illustrates effects of amino acid substitutions on predicted glycosylation ranges. For all N-linked sequons in the dataset, all amino acids were systematically substituted with every other amino acid and the modified sequons were used as input for a trained algorithm as described herein, obtaining a predicted glycosylation range. The average change was then calculated and compared to the glycosylation range of the wild-type sequons, which is depicted with a 95% confidence interval. Lines for changes to high-mannose (“g”), fucosylated (“r”), and sialylated (“p”) glycans are shown, with a horizontal line at zero.

FIGS. 20A-20B show distributions of predicted-presence for the L and F variants at N-2 stratified by mannose per glycan (FIG. 20A) and sialic acids per glycan (FIG. 20B). FIG. 20C illustrates simpler boxplots which describe predicted glycosylation by mannose per glycan and sialic acid per glycan for three oligomannose sites in the SARS-CoV-2 spike glycoprotein. FIG. 20D shows glycan predict-presence fold-changes at site N717 (by galactose and sialic acid between the wild-type and B.1.1.7 spike protein. Predicted-presence fold-change (y-axis) is stratified by the basal predicted-presence for each glycan in the wild-type (x-axis). Predicted-presence fold-change from wild-type by galactose, mannose, GlcNAc, and sialic acid is provided for N717 and N616 in B.1.1.7 and D615G variants respectively. ns: p>0.05, *: p<0.05, **: p<0.01, **: p<0.001, ***: p<1e−3, ****:p<1e−4

FIG. 21 shows predicted-presence by monosaccharide for all sites in the SARS-CoV-2 spike. Glycosylation by mannose per glycan and sialic acid per glycan. Bottom bar indicates the dominant glycan type at each site, hybrid (122, 603), oligomannose (61, 234, 717, 801), complex (remainder) characterized at that site in the wildtype spike.

FIGS. 22A-D shows predicted change in presence for glycans at N616 in D614G. Predicted-presence fold-change (y-axis) is stratified by the basal predicted-presence for each glycan in the wild-type (x-axis). Predicted-presence fold-change from wild-type by galactose (FIG. 22A), mannose (FIG. 221B), GlcNAc (FIG. 22C), and sialic acid (FIG. 22D) is provided for N616 in D614G.

FIG. 23A depicts a heatmap showing the log-scale abundance of various glycan species observed in wt and mutant Fe on human IgG3. FIG. 23B shows the background-adjusted InSaNNe predicted-presence compared with the empirical abundance in wild type (circle), R301A mutant (“+”), and the Y296A mutant (“x”). FIG. 23C shows the log fold change between glycan abundance for mutants relative to wildtype were compared between empirical and predicted abundance for all glycans. FIGS. 23D-23E mirror FIGS. 23B-23C except glycans with a predicted absolute log fold-change less than 1 were removed.

FIG. 24 depicts a table showing random forest model performance in a 2×6-fold cross-validation. Cross validation folds were split on protein identity. Training data were either annotated from SWISSMOD curated homology models, PDB empirical models, or ab initio I-TASSER homology models using structural resolutions between 4-10 Å. Performance was measured using average AUROC, Sensitivity and Specificity across cross-validation and the standard error across 12 folds in parenthesis. For each model type, the top two scores are bold. Rows with two or more top scores are noted in the final column.

FIG. 25 depicts a table showing ablation of major protein structure feature types. Seven ablations of major feature-types including: struct (all structure-derived annotation), Depth & Accessibility (depth of residue, relative/absolute surface area), SS (secondary structure), aaUp/Down/All (sequence-proximal amino acids upstream/downstream/either), structAA (structure-proximal amino acids). Because some feature-types are associated, the ablation of some feature-types such as aaUp also required the removal of other feature-types indicated by the “x” left of the center line. Ablation significance is indicated by FDR-corrected Fisher's Method pooled p-values (2-sample t-test, n=12 for each sample) comparing the performance distribution of ablation trained models to models trained on all data; performance distributions were collected over 2×6-fold cross-validation.

FIG. 26A shows the validation loss as a function of training epoch of a trained algorithm as described herein. FIG. 26B shows the validation accuracy as a function of training epoch of a trained algorithm as described herein.

FIG. 27 shows a computer control system that is programmed or otherwise configured to implement methods provided herein.

FIG. 28A depicts a plot showing false positive rate estimation of a trained algorithm as described herein. For classification thresholds between 0 and 1, the true and false positive rate of InSaNNe predictions was assessed on the independent test set and compared to a random classifier baseline. FIG. 28B shows validation of predictions by the trained algorithm with existing structures on GlyConnect. The influence of classification threshold on the hit rate (recall/sensitivity) was investigated by evaluating whether recorded structures on GlyConnect would have been predicted by InSaNNe. FIG. 28C illustrates observed compositions in GlyConnect via predicted structures from InSaNNe. For each composition, the predicted probability of structures with that composition was probed to evaluate whether the composition was predicted via matching structures.

FIG. 29 shows a dependence of sequon prediction performance on occurrence. Using a trained model as described herein (InSaNNe), the prediction performance of sequons, averaged over all their observed glycans, was determined and plotted against the number of sequon-glycan pairs in the dataset.

FIG. 30 shows the redundancies in sequon sequence. All sequon-glycan pairs in the dataset were used to obtain an averaged recall value for the trained InSaNNe model (WT), compared to an averaged recall value when removing the entire sequon sequence (all). Single or multiple amino acid positions were then iteratively removed from all sequons and model recall assessed.

### DETAILED DESCRIPTION OF THE INVENTION

Unless defined otherwise, all terms of art, notations and other technical and scientific terms or terminology used herein are intended to have the same meaning as is commonly understood by one of ordinary skill in the art to which the claimed subject matter pertains. In some embodiments, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5. from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some embodiments, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

“Position” refers to a particular amino acid by its index relative to a contextual zero-position, e.g., the first amino acid at the N-terminal end of the molecule.

“Region” refers to a portion of an amino acid or nucleic acid, wherein said portion is smaller than the entire amino acid or nucleic acid.

The terms “identical” or percent “identity” in the context of two or more nucleic acid or polypeptide sequences, refer to two or more sequences or subsequences that are the same or have a specified percentage of amino acid residues or nucleotides that are the same, when compared and aligned for maximum correspondence, e.g., as measured using one of the sequence comparison algorithms available to persons of skill or by visual inspection.

Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated by reference. Many other optimal alignment algorithms are also known in the art and are optionally utilized to determine percent sequence identity.

### Sequences and Sequons

Methods and systems as described herein may ingest as inputs or operate on one or more sequences of biological molecules. Sequences may comprise amino acid or nucleic acid sequences. Sequences as described herein may be distinguished according to one or more property or features. The feature may comprise sequence length; amino acid identity (e.g., presence or absence of a specific amino acid or content of a specific amino acid); amino acid position (e.g., relative or absolute position of an amino acid or amino acids); amino acid insertions (e.g., relative to a reference sequence); amino acid deletions (e.g., relative to a reference sequence); amino acid substitutions (e.g., relative to a reference sequence); observed or predicted structure of the sequence, including observed or predicted secondary structure (e.g., 3-turn helix, 4-turn (alpha) helix, 5-turn (pi) helix, beta strand, bend, (random) coil), observed or predicted tertiary structure, and observed or predicted quaternary structure; observed or predicted glycosylation features associated with the sequence; or any combination thereof.

In some embodiments, an amino acid “sequence” (sometimes referred to herein as simply a “sequence”) of a peptide refers to the order and identity of amino acids in the peptide (wherein peptide is inclusive of proteins). In some embodiments, an amino acid sequence comprises a glycosite, referring to an amino acid of the sequence that is, has the potential to be, or is predicted to be, attached via a hydroxyl or other functional group of that amino acid to a glycan (e.g., a glycan comprising one or more glycosylation features) to form a glycoconjugate. In some embodiments, a sequence comprising a glycosite comprises amino acids that are structurally proximal (“structurally proximal amino acids” or “structurally proximal AAs”) to the glycosite. For example, in some embodiments amino acids that are structurally proximal include those amino acids that are within 1, 2, 3, 4, 5, 6, 7, 8, 9, 10, 11, 12, 13, 14, 15, 16, 17, 18, 19, 20, 30, or more Angstroms of the glycosite when the monomeric peptide sequence is arranged in three-dimensional space to have secondary, tertiary, and/or quaternary structure. In some embodiments, a sequence comprising a glycosite comprises sequence proximal amino acids, where sequence proximal amino acids are amino acids that are within 20 amino acids upstream or downstream of the glycosite. For instance, for the non-limiting example amino acid sequence AA1-AA2-AA3-AA4-AA5-AA6-AA7-AA8-AA9-AA10-AA11-AA12-AA13-AA14-AA15-AA16-AA17-AA18-AA19-AA20-AA21-AA22-AA23-AA24-AA25 (N)-AA26-AA27-AA28-AA29-AA30-AA31-AA32-AA33-AA34-AA35-AA36-AA37-AA38-AA39-AA40-AA41-AA42-AA43-AA44-AA45-AA46-AA47-AA48-AA49-AA50, where each “AA” is an amino acid, AA25 is a glycosite (N), and amino acids AA4-AA24 and AA26-AA46 are “sequence proximal”. Accordingly, use of the term “sequence” is inclusive of an amino acid sequence comprising a glycosite with one or more amino acids that are structurally proximal and sequence proximal to the glycosite.

In some embodiments, a sequence comprises a sequon, which comprises a glycosite. In some cases, the sequon comprises a glycosite and one or more structurally proximal amino acids. In some cases, the sequon comprises a glycosite and one or more sequence proximal amino acids. In some cases, the sequon comprises a glycosite and one or more structurally proximal amino acids, and one or more sequence proximal amino acids. While sequon has been referred to as N-type NX[S/T], in various embodiments it is used herein to include the sequence and structure context surrounding any glycosite.

In some embodiments, a sequence or sequons comprises one or more observed or predicted glycosylation sites and any number of flanking residues (e.g., amino acids). In some embodiments, a sequence or sequon comprises a glycosylation site and about 1, 2, 3, 4, 5, 6, 7, 8, 9, 10, 11, 12, 13, 14, 15, 16, 17, 18, 19, 20, 30, or more residues flanking the glycosylation site (glycosite). The flanking residues may be in either or both directions of the glycosylation site (or glycosite) (e.g., upstream, downstream, N-terminal. C-terminal). The flanking residues may comprise any monomer. In some embodiments, an N-type glycosite in a sequence or sequence, comprises an NX[S/T] motif where N is an asparagine residue which may or may potentially be glycosylated, X is any amino acid except proline, S is serine, and T is threonine. In some embodiments, a sequence or sequon comprises an extended aromatic sequon (EAS). In some embodiments, a sequence or sequon comprises an O-type glycosite, and may comprise a glycosylated Serine or Threonine and flanking amino acids. In some embodiments, a sequence may have associated with it one or more glycosites as described elsewhere herein. In some embodiments, a sequence of sequon may comprise a glycosite, amino acids flanking the glycosite (sequence proximal), and physically proximal amino acid residues within about 1, 2, 3, 4, 5, 6, 7, 8, 9, 10, 11, 12, 13, 14, 15, 16, 17, 18, 19, 20, 30, or more Angstroms from the glycosylated amino acid (or glycosite).

### Glycosylation Features

Glycoconjugates (e.g., a glycopeptide) as described herein may comprise one or more glycosylation features or glycans decorating a glycosite of an amino acid sequence. A glycosylation feature may comprise one or more monosaccharides linked glycosidically. A glycosylation feature may be present or otherwise associated with the glycosite. The association may comprise one or more covalent (e.g., glycosidic) bonds or the association may be non-covalent. A glycosylation feature may comprise any number of monosaccharides or derivatives. A glycosylation feature may comprise 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more monosaccharides or derivatives thereof.

Glycosylation features as described herein may comprise any monosaccharide or derivative thereof. Monosaccharides may comprise D-glucose (Glc), n-galactose (Gal), N-acetylglucosamine (GlcNAc), N-acetylgalactosamine (GalNAc), D-mannose (Man), N-acetylneuraminic acid (Neu5Ac), N-glycolylneuraminic acid (Neu5Gc), neuraminic acid (Neu), 2-keto-3-deoxynononic acid or 3-deoxy-D-glycero-D-galacto-nonulosonic acid (KDN), 3-deoxy-D-manno-2 octulopyranosylonic acid (KdO), D-galacturonic acid (GalA), L-iduronic acid (IdoA), L-rhamnose (Rha), L-fucose (Fuc), D-xylose (Xyl), D-ribose (Rib), L-arabinofuranose (Araf), D-glucuronic acid (GlcA), D-allose (All), D-apiose (Api), D-fructofuranose (Fruf), ascarylose (Asc), and ribitol (Rbo). Derivatives of monosaccharides may comprise sugar alcohols, amino sugars, uronic acids, ulosonic acids, aldonic acids, aldaric acids, sulfosugars, or any combination or modification thereof. A sugar modification may comprise one or more of acetylation, propylation, formylation, phosphorylation. or sulfonation or addition of one or more of deacetylated N-acetyl (N), phosphoethanolam ine (Pe), inositol (In), methyl (Me), N-acetyl (NAc), O-acetyl (Ac), phosphate (P), phosphocholine (Pc), pyruvate (Pyr), sulfate (S), sulfide (Sh), aminoethylphosphonate (Ep), deoxy (d), carboxylic acid (-oic), amine (-amine), amide (-amide), and ketone (-one). Such modifications may be present at any position on the sugar, as designated by standard sugar naming/notation. In some cases, a glycosytic addition of a monosaccharide to another monosaccharide is considered a polymerizing modification that gives rise to a glycans. In some embodiments, at least 1, 2, 3, 4, 5, 6, 7, 8, 9, 10 or more modifications are present on the monosaccharide. In some embodiments, no more than 10, 9, 8, 7, 6, 5, 4, 3, 2, 1, or fewer modifications are present on the monosaccharide. Monosaccharides may comprise any number of carbon atoms. Monosaccharides may comprise any stereoisomer, epimer, enantiomer, or anomer. In some embodiments, monosaccharides comprise 3, 4, 5, 6, 7, 8, 9, 10, 11, 12, 13, 14, 15, or more carbon atoms.

In some embodiments, a glycosylation feature comprises glyceraldehyle, threose, erythrose, lyxose, xylose (Xyl), arabinose, ribose, talose, galactose (Gal), idose, gulose, mannose (Man), glucose (Glc), altrose, allose, sedoheptulose, mannoheptulose, N-acetyl-galactosamine (Glc2NAc), glucuronic acid (GlcA), 3-O-sulfogalactose (Gal3S), N-acetylneuraminic acid (Neu5Ac), 2-keto-3-deoxynonic acid (Kdn), and any combination thereof.

A glycosylation feature may comprise one monosaccharide. A glycosylation feature may comprise a plurality of monosaccharides. In such cases, the monosaccharides may be connected in any configuration through any suitable glycosidic bond(s). Glycosidic bonds between monosaccharides in a polysaccharide glycosylation feature may be alpha or beta and connect any two carbon atoms between adjacent monosaccharide residues through an oxygen atom. In some embodiments, the glycosylation feature of glycan is an N-linked, O-linked, C-linked, or S-linked glycan. In some embodiments, more than one glycosylation feature is present on a single biomolecule. The more than one glycosylation features may all be linked in the same manner (e.g., N-linked, O-linked, C-linked, S-linked), or they may be independently N-linked, O-linked, C-linked, or S-linked. Glycosylation features may be branched, linear, or both. Glycosylation features may be biantennary, triantennary, tetra-antennary, or any combination thereof. In some embodiments, the glycosylation feature comprises a polysaccharide epitope. In some embodiments, the glycosylation feature comprises high-mannose. In some embodiments, the glycosylation feature comprises sialylation. In some embodiments, the glycosylation feature comprises fucosylation. In some embodiments, the glycosylation feature comprises hybrid, complex, core or distally fucosylated, terminally sialylated, terminally galactosylated, terminally GlcNAc-ylated, GlcNAc-bisected, or poly-sialylated, or a combination thereof.

A glycosylation feature may be described in relative terms. A glycosylation feature may be described as increased or decreased with respect to the amount of a given monosaccharide in the glycosylation feature relative to a reference glycosylation feature. For example, a glycosylation feature may be described as an increase or increased in sialylation or fucosylation if the glycosylation feature comprises more sialic acid or fucose residues, respectively, than a reference glycan. Alternatively or additionally, a glycosylation feature may be described as increased or decreased with respect to the configuration (e.g., branched, linear, biantennary, tri-antennary, tetra-antennary, penta-antennary) of the glycosylation feature relative to a reference glycosylation feature. For example, a glycosylation feature may be described as an increase or increased in branching if the glycosylation feature comprises more branches than a reference glycosylation feature. In some embodiments, a glycosylation feature may be described as increased or decreased in one or more of high-mannose, sialylation, fucosylation, hybrid, complexity, core or distally fucosylation, terminal sialylation, terminal galactosylation, terminal GlcNAc-ylation, GlcNAc-bisection, or poly-sialylation, or a glycosylation feature listed in Table 1.

In some embodiments, a glycosylation feature is one of those listed in Table 1. Table 1. Table of representative glycosylation features. Glycosylation features are described using IUPAC-extended format (doi.org/10.1351/pac199668101919). Table 1 can be found at https://doi.org/10.5281/zenodo.6459738

### Glycosites

Methods and systems as described herein may analyze one or more glycosites. Glycosites may comprise any site on a molecule (e.g., protein, lipid, nucleic acid) that can be glycosylated, whether or not the site is glycosylated. Generally, such sites comprise one or more atoms (e.g., nitrogen, oxygen, sulfur, carbon), optionally in one or more moieties (e.g., amino, amido, phenol, hydroxyl, guanidino, alcohol, thiol, indole), that are capable of forming a glycosidic bond with a sugar (e.g., glycosylation feature, such as a monosaccharide, oligosaccharide, polysaccharide, or derivative) molecule or part thereof. In some embodiments, a glycosite may comprise an amino acid comprising a side chain comprising an oxygen atom. In some embodiments, a glycosite may comprise an amino acid comprising a side chain comprising a sulfur atom. a glycosite may comprise an amino acid comprising a side chain comprising a nitrogen atom. The glycosite may comprise arginine, asparagine, serine, threonine, tyrosine, cysteine, homocysteine, omithine, or lysine. In some embodiments, a glycosite may comprise a nucleic acid or portion (e.g., nucleotide) thereof. In some embodiments, a glycosite may comprise a lipid or portion thereof.

Glycosites may be further distinguished by the sequence or sequon comprising the glycosite. In some embodiments, sequence or sequon may comprise other atoms, moieties, residues, monomers (e.g., amino acids, monosaccharides), or glycosites or glycan features in proximity to an atom forming or capable of forming a glycosidic bond. For example, a glycosite may be designated based on the sequential or spatial proximity of a particular amino acid or nucleoside to an atom that may be or may potentially be glycosylated. Proximity may be described in relative terms (e.g., C-terminal or N-terminal to a reference amino acid), absolute terms (e.g., within three sites of a reference amino acid, within 6 Å of an amino acid), or a combination thereof (e.g., within three site C-terminal to a reference amino acid).

In some embodiments, a sequence or sequon may comprise one or more amino acids within a certain sequence position. The one or more amino acids may be within 1, 2, 3, 4, 5, 6, 7, 8, 9, 10, 11, 12, 13, 14, 15, 16, 17, 18, 19, 20, or more positions of the atom forming or capable of forming the glycosidic bond. The one or more amino acids may be within no more than 20, 19, 18, 17, 16, 15, 14, 13, 12, 11, 10, 9, 8, 7, 6, 5, 4, 3, 2, or 1 positions of the atom forming or capable of forming the glycosidic bond. The one or more amino acids may be C-terminal with respect to the glycosite. The one or more C-terminal amino acids may be within 1, 2, 3, 4, 5, 6, 7, 8, 9, 10, 11, 12, 13, 14, 15, 16, 17, 18, 19, 20, or more positions of the atom forming or capable of forming the glycosidic bond. The one or more amino acids may be N-terminal with respect to the glycosite. The one or more N-terminal amino acids may be within 1, 2, 3, 4, 5, 6, 7, 8, 9, 10, 11, 12, 13, 14, 15, 16, 17, 18, 19, 20, or more positions of the atom forming or capable of forming the glycosidic bond. The one or more amino acids may comprise an atom within about 1, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.5, about 7.0, about 7.5, about 8.0, about 8.5, about 9.0, about 9.5, about 10.0, about 10.5, about 11.0, about 11.5, about 12.0, about 13.0, about 14.0, about 15.0, about 16.0, about 17.0, about 18.0, about 19.0, about 20.0, about 21.0, about 22.0, about 23.0, about 24.0, about 25.0, about 26.0, about 27.0, about 28.0, about 29.0, about 30.0 Å or more of the atom forming or capable of forming a glycosidic bond. The one or more amino acids may comprise an atom within about 30.0, about 29.0, about 28.0, about 27.0, about 26.0, about 25.0, about 24.0, about 23.0, about 22.0, about 21.0, about 20.0, about 19.0, about 18.0, about 17.0, about 16.0, about 15.0, about 14.0, about 13.0, about 12.0, about 11.5, about 11.0, about 10.5, about 10.0, about 9.5, about 9.0, about 8.5, about 8.0, about 7.5, about 7.0, about 6.5, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1 Å or less of the atom forming or capable of forming a glycosidic bond.

A glycosite may be distinguished based on the sequence or sequon containing the glycosite. In some embodiments, a sequence or sequon may comprise a glycosite occurrence within or near a particular protein structural element. In some embodiments, the glycosite may be in a particular secondary structural element. In some embodiments, the secondary structural element comprises one or more of alpha-helix (H), 3₁₀-helix (G), pi-helix (I), extended strand (E), beta-bridge (B), turn (T), bend (S), or random coil (C). In some embodiments, the glycosite may be within one or more sites of the secondary structural element. In some embodiments, the glycosite may be within 1, 2, 3, 4, 5, 6, 7, 8, 9, 10, 11, 12, 13, 14, 15, 16, 17, 18, 19, 20, or more positions of the secondary structural element. In some embodiments, the glycosite may be within about 1, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, 4 about 0.8, about 4.9, about 5.0, about 5.1, about 5.2, about 5.3, about 5.4, about 5.5, about 5.6, about 5.7, about 5.8, about 5.9, about 6.0, about 6.5, about 7.0, about 7.5, about 8.0, about 8.5, about 9.0, about 9.5, about 10.0, about 10.5, about 11.0, about 11.5, about 12.0, about 13.0, about 14.0, about 15.0, about 16.0, about 17.0, about 18.0, about 19.0, about 20.0, about 21.0, about 22.0, about 23.0, about 24.0, about 25.0, about 26.0, about 27.0, about 28.0, about 29.0, about 30.0 Å or more of the secondary structural element. In some embodiments, the glycosite may be within no more than about 30.0, about 29.0, about 28.0, about 27.0, about 26.0, about 25.0, about 24.0, about 23.0, about 22.0, about 21.0, about 20.0, about 19.0, about 18.0, about 17.0, about 16.0, about 15.0, about 14.0, about 13.0, about 12.0, about 11.5, about 11.0, about 10.5, about 10.0, about 9.5, about 9.0, about 8.5, about 8.0, about 7.5, about 7.0, about 6.5, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1 Å or less of the secondary structural element. In some embodiments, the glycosite may be in a particular tertiary structural element. In some embodiments, the glycosite may be within one or more sites of the tertiary structural element. In some embodiments, the glycosite may be within 1, 2, 3, 4, 5, 6, 7, 8, 9, 10, 11, 12, 13, 14, 15, 16, 17, 18, 19, 20, or more positions of the tertiary structural element. In some embodiments, the glycosite may be within about 1, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, 4 about 0.8, about 4.9, about 5.0, about 5.1, about 5.2, about 5.3, about 5.4, about 5.5, about 5.6, about 5.7, about 5.8, about 5.9, about 6.0, about 6.5, about 7.0, about 7.5, about 8.0, about 8.5, about 9.0, about 9.5, about 10.0, about 10.5, about 11.0, about 11.5, about 12.0, about 13.0, about 14.0, about 15.0, about 16.0, about 17.0, about 18.0, about 19.0, about 20.0, about 21.0, about 22.0, about 23.0, about 24.0, about 25.0, about 26.0, about 27.0, about 28.0, about 29.0, about 30.0 Å or more of the tertiary structural element. In some embodiments, the glycosite may be within no more than about 30.0, about 29.0, about 28.0, about 27.0, about 26.0, about 25.0, about 24.0, about 23.0, about 22.0, about 21.0, about 20.0, about 19.0, about 18.0, about 17.0, about 16.0, about 15.0, about 14.0, about 13.0, about 12.0, about 11.5, about 11.0, about 10.5, about 10.0, about 9.5, about 9.0, about 8.5, about 8.0, about 7.5, about 7.0, about 6.5, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1 Å or less of the tertiary structural element. In some embodiments, the glycosite may be in a particular quaternary structural element. In some embodiments, the glycosite may be within one or more sites of the quaternary structural element. In some embodiments, the glycosite may be within 1, 2, 3, 4, 5, 6, 7, 8, 9, 10, 11, 12, 13, 14, 15, 16, 17, 18, 19, 20, or more positions of the quaternary structural element. In some embodiments, the glycosite may be within about 1, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, 4 about 0.8, about 4.9, about 5.0, about 5.1, about 5.2, about 5.3, about 5.4, about 5.5, about 5.6, about 5.7, about 5.8, about 5.9, about 6.0, about 6.5, about 7.0, about 7.5, about 8.0, about 8.5, about 9.0, about 9.5, about 10.0, about 10.5, about 11.0, about 11.5, about 12.0, about 13.0, about 14.0, about 15.0, about 16.0, about 17.0, about 18.0, about 19.0, about 20.0, about 21.0, about 22.0, about 23.0, about 24.0, about 25.0, about 26.0, about 27.0, about 28.0, about 29.0, about 30.0 Å or more of the quaternary structural element. In some embodiments, the glycosite may be within no more than about 30.0, about 29.0, about 28.0, about 27.0, about 26.0, about 25.0, about 24.0, about 23.0, about 22.0, about 21.0, about 20.0, about 19.0, about 18.0, about 17.0, about 16.0, about 15.0, about 14.0, about 13.0, about 12.0, about 11.5, about 11.0, about 10.5, about 10.0, about 9.5, about 9.0, about 8.5, about 8.0, about 7.5, about 7.0, about 6.5, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1 Å or less of the quaternary structural element.

In some embodiments, a sequence or sequon may comprise a glycosite occurrence near another glycosite. In some embodiments, the glycosite may be within 1, 2, 3, 4, 5, 6, 7, 8, 9, 10, 11, 12, 13, 14, 15, 16, 17, 18, 19, 20, or more positions (e.g., amino acid residues) of the other glycosite. In some embodiments, the glycosite may be within about 1, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, 4 about 0.8, about 4.9, about 5.0, about 5.1, about 5.2, about 5.3, about 5.4, about 5.5, about 5.6, about 5.7, about 5.8, about 5.9, about 6.0, about 6.5, about 7.0, about 7.5, about 8.0, about 8.5, about 9.0, about 9.5, about 10.0, about 10.5, about 11.0, about 11.5, about 12.0, about 13.0, about 14.0, about 15.0, about 16.0, about 17.0, about 18.0, about 19.0, about 20.0, about 21.0, about 22.0, about 23.0, about 24.0, about 25.0, about 26.0, about 27.0, about 28.0, about 29.0, about 30.0 Å or more of the other glycosite. In some embodiments, the glycosite may be within no more than about 30.0, about 29.0, about 28.0, about 27.0, about 26.0, about 25.0, about 24.0, about 23.0, about 22.0, about 21.0, about 20.0, about 19.0, about 18.0, about 17.0, about 16.0, about 15.0, about 14.0, about 13.0, about 12.0, about 11.5, about 11.0, about 10.5, about 10.0, about 9.5, about 9.0, about 8.5, about 8.0, about 7.5, about 7.0, about 6.5, about 6.0, about 5.9, about 5.8, about 5.7, about 5.6, about 5.5, about 5.4, about 5.3, about 5.2, about 5.1, about 5.0, about 4.9, about 4.8, about 4.7, about 4.6, about 4.5, about 4.4, about 4.3, about 4.2, about 4.1, about 4.0, about 3.9, about 3.8, about 3.7, about 3.6, about 3.5, about 3.4, about 3.3, about 3.2, about 3.1, about 3.0, about 2.9, about 2.8, about 2.7, about 2.6, about 2.5, about 2.4, about 2.3, about 2.2, about 2.1, about 2.0, about 1.9, about 1.8, about 1.7, about 1.6, about 1.5, about 1 Å or less of the other glycosite.

In some embodiments, a sequence of sequon may comprise predicted or observed solvent accessible surface area (SASA), total solvent accessibility (ASA), relative solvent accessibility (RSA), or any combination thereof. In some embodiments, a glycosite may be distinguished on the basis of its hydrophobicity. The hydrophobicity of a residue or residues may be estimated or determined by, for example, a Kyte-Doolittle scale. In some embodiments, a glycosite may be distinguished on the basis on the depth of a residue center or atom (e.g., alpha carbon, such as of an amino acid). In some embodiments, a glycosite may be distinguished on the basis of one or more dihedral angles. In some embodiments, the dihedral angles comprise phi and psi angles of an amino acid residue. In some embodiments, the dihedral angles comprise one or more torsion angles (e.g., alpha, beta, gamma, delta, epsilon, zeta, nu) of a nucleic acid residue.

### Intermolecular Relationships

Methods and systems as described herein may produce or use parameters characterizing the association between glycosylation features and biomolecular features (e.g., amino acids proximal to the glycosite by sequence and/or three-dimensional space, such as found in a sequence of sequon comprising a glycosite). Such parameters may characterize the strength of an association between a glycosylation feature or aspect of a glycosylation feature and a biomolecule or aspect of a biomolecule. These parameters may be referred to herein as “intermolecular relations” or “IMRs.”

In some embodiments, the parameter characterizes a relationship (e.g., an association) of a glycosylation feature or part thereof as described herein. In some embodiments, the relationship is an association with a biomolecular feature or a glycosite as described herein. The glycosite may be characterized by one or more of glycosylation feature, biomolecular sequence (e.g., amino acid sequence), biomolecule composition (e.g., amino acid identity), amino acid substitution, amino acid position, observed or predicted structure (e.g., secondary, tertiary, quaternary structure), presence or absence of another glycosite, proximity to another glycosite, glycosylation composition, glycosylation configuration (e.g., linear, branched), hydrophobicity, solvent accessibility, and any combination thereof.

The IMRs may be determined from one or more datasets comprising sets of biomolecular features and glycosylation features. The datasets may comprise data from one or more databases of experimental structural biology data (e.g., X-ray crystallographic data, cryogenic electron microscopy data, nuclear magnetic resonance data), experimental glycan measurements or biochemical data (e.g., mass spectrometry data, chromatographic data), computer simulation or modeling data (e.g., molecular dynamics simulations, de novo or ab initio prediction, homology modeling, fragment assembly, secondary structure prediction algorithms, trained algorithms as described herein). By observing the coincidence of certain biomolecular features and corresponding glycosylation features, IMRs may be determined. Alternatively or additionally, the IMRs may be determined by a trained algorithm as described herein. The trained algorithm may be able to offer a robust, noise-mitigated observation or prediction of IMRs.

Any suitable measure of association may be used to determine the association between a biomolecular feature and a glycosylation feature. In some embodiments, the measure of association is determined by a generalized estimating equation (GEE), a Fisher exact test, a chi-squared test, or a hypergeometric test. In some embodiments, the determination of the association may comprise a dimensionality reduction process. The dimensionality reduction process may comprise principle component analysis (PCA), kernel PCA, linear discriminant analysis (LDA), independent component analysis (ICA), non-negative matrix factorization (NMF), uniform manifold approximation and projection (UMAP), t-distributed stochastic neighbor embedding (tSNE), or neural network embedding. In some embodiments, the IMRs may be expressed as an odds ratio. In some embodiments, the IMRs may be expressed as a logarithm of an odds ratio. In some cases, the odds ratio is determined by GEE. In some cases, the odds ratio is determined by Fisher's exact test.

IMRs as described herein may be either positive or negative. In some embodiments, an IMR may be positive. In such cases, the IMR may indicate that a glycosylation feature and a sequence, sequon, glycosite, or part thereof, tend to occur together (e.g., the sequence, sequon, or glycosite, or part thereof, is associated with the presence of the glycosylation feature). In some embodiments, an IMR may be negative. In such cases, the IMR may indicate that a glycosylation feature and a sequence, sequon, or glycosite, or part thereof, tend not to occur together (e.g., the sequence, sequon, or glycosite, or part thereof, is associated with the absence of the glycosylation feature). In some embodiments, an IMR may be zero. In such cases, the IMR may indicate the glycosylation feature and sequence, sequon, or glycosite, or part thereof, are independent (e.g., are not associated).

IMRs may have associated with them one or more measures of uncertainty. The measures of uncertainty may comprise confidence intervals, estimated errors, estimated standard errors of the mean, p-values, or false discovery rates (FDRs) or corrections thereto.

In some embodiments, the IMR may be described as “significant” is it has a measure of uncertainty that is above or below a cutoff or threshold value. In some embodiments, an IMR may be described as “significant” if it is associated with a p-value less than a cutoff value. In some embodiments, the cutoff value is less than about 1.0, about 0.9, about 0.8, about 0.7, about 0.6, about 0.5, about 0.4, about 0.3, about 0.2, about 0.1, about 0.09, about 0.08, about 0.07, about 0.06, about 0.05, about 0.04, about 0.03, about 0.02, about 0.01, about 0.005, about 0.0001, about 0.0005, about 0.0001, or less. In some embodiments, an IMR may be described as “significant” if it is associated with an FRD correction less than a cutoff value. In some embodiments, the cutoff value is less than about 1.0, about 0.9, about 0.8, about 0.7, about 0.6, about 0.5, about 0.4, about 0.3, about 0.2, about 0.1, about 0.09, about 0.08, about 0.07, about 0.06, about 0.05, about 0.04, about 0.03, about 0.02, about 0.01, about 0.005, about 0.001, about 0.0005, about 0.0001, or less.

In some embodiments, the IMR may be described as “significant” if its magnitude is above a cutoff or threshold value. In some embodiments, the threshold is at least about 0.0001, about 0.001, about 0.01, about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1.0, about 2.0, about 3.0, about 4.0, about 5.0, about 10.0, about 100.0, or more. In, some embodiments, the IMR may be described as “significant” if the threshold is at least about 1.0 or more. In, some embodiments, the IMR may be described as “significant” if the threshold is at least about 0.1 or more.

In some embodiments, the IMR may be described as “moderate” if its magnitude is above a lower cutoff or threshold value but is less than some upper cutoff or threshold value. In some embodiments, the lower threshold is at least about 0.0001, about 0.001, about 0.01, about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1.0, about 2.0, about 3.0, about 4.0, about 5.0, about 10.0, about 100.0, or more. In some embodiments, the upper threshold is no more than about 100.0, about 10.0, about 5.0, about 4.0, about 3.0, 2 about 0.0, about 1.0, about 0.95, about 0.9, about 0.85, about 0.8, about 0.75, about 0.7, about 0.65, about 0.6, about 0.55, about 0.5, about 0.45, about 0.4, about 0.35, about 0.3, about 0.25, about 0.2, about 0.15, about 0.1, about 0.01, about 0.001, about 0.0001, or less. The IMR may be described as “moderate” if the threshold is at least about 0.5 but less than about 1.0.

In some embodiments, the IMR may be described as “weak” if its magnitude is below a cutoff or threshold value. In some embodiments, the threshold is no more than about 100.0, about 10.0, about 5.0, about 4.0, about 3.0, 2 about 0.0, about 1.0, about 0.95, about 0.9, about 0.85, about 0.8, about 0.75, about 0.7, about 0.65, about 0.6, about 0.55, about 0.5, about 0.45, about 0.4, about 0.35, about 0.3, about 0.25, about 0.2, about 0.15, about 0.1, about 0.01, about 0.001, about 0.0001, or less. The IMR may be described as “weak” if the threshold is less than about 0.1.

Methods and systems as described herein may take as inputs or process one or more IMRs. IMRs may be used in methods of determining the effect of sequence modification on glycosylation, methods of modifying glycopeptides, methods of modifying other biomolecules, and any combination thereof. Methods and systems which take as input of process one of more IMRs may comprise an operation on the one or more IMRs. In some embodiments, the operation may comprise calculating a norm of two IMRs. In some embodiments, the norm is an -norm. In some embodiments, the norm comprises a Euclidean norm (also referred to herein as a “Euclidean distance” or “-norm”). In some embodiments, IMRs may be expressed or encoded in one or more substitution matrices (i.e., BLAMO). Additional example substitution matrix may comprise a point accepted mutation (PAM) matrix, a block substitution matrix (BLOSUM), or any combination or variant thereof.

In some embodiments, an IMR comprises an IMR as listed in Table 2. In some embodiments, an IMR comprises an IMR as listed in Table 3. The first column of Table 2 and Table 3, “Protein_Structure,” specify a glycan-associated protein structure. The prefix for the protein structure variables is as follows, “<glycan_type>_<seq/struc>_<measurement>_”. The “glycan_type” will be shown as “ASN” (indicating an N-glycan associated protein structure) or “SER.THR” (indicating an 0-glycan associated protein structure). The “seq_” and “struc_” prefix indicate if the measure was derived from sequence or structure respectively. The “measurement” term indicates the type of protein structure descriptor measurement. The “measurement” can refer to proximal amino acids as “_aa_” (occurrences of a specific amino acid within n Angstroms glycosite), “_aaUp_” (occurrences of a specific amino acid within n positions of a glycosite upstream), “_aaDown_” (occurrences of a specific amino acid within n positions of a glycosite downstream), “_aaAll_” (occurrences of a specific amino acid within n positions of a glycosite upstream or downstream). When the measurement refers to amino acids, the suffix will be “_<aa>” where aa is a one letter expression of the proximal amino acid. The “measurement” can refer to secondary structure as “_SS_” (secondary structure predicted either from sequence or 3D structure). When the “measurement” refers to secondary structure, secondary structure can be predicted from sequence using the sspro or sspro8 tools in the SCRATCH protein prediction software indicated by the suffix “_sspro<ss>” or “sspro8<ss>” where ss is a secondary structure output by sspro8 (H: alpha-helix G: 310-helix I: pi-helix (extremely rare) E: extended strand B: beta-bridge T: turn S: bend C: the rest) or sspro (H: helix E: strand C: the rest). Secondary structure can also be predicted from 3D structure using DSSP software indicated by the suffix “_dssp<ss>” where ss is a secondary structure output by dssp (H: Alpha helix, B: Beta bridge, E: Strand, G: Helix-3, I: Helix-5, T: Turn, S: Bend). The “measurement” can refer to hydrophobicity (Kyte-Doolitle hydrophobicity based on 7 glycosite flanking amino acids) indicated by the “hydrophobicity_kd” suffix. The “measurement” can refer to alpha-carbon or whole-residue depth as “_CA depth_” or “_RES_depth_” respectively. When the “measurement” refers to depth, depth can be predicted from 3D structure using MSMS software indicated by the “_msms” suffix. The “measurement” can refer to relative and absolute solvent accessibility and solvent-accessible surface area as “_RSA_” or “ASA_” respectively. When the “measurement” refers to absolute solvent accessibility, absolute solvent-accessible surface area (ASA) can be predicted from 3D protein structure using the FreeSASA software indicated by the “_[asa]_freesasa_het” suffix where asa specifies either all, backbone, polar, apolar, or residue accessibility; ASA can also be calculated from 3D protein structure using the DSSP software indicated by the “_dssp” suffix. When the “measurement” refers to relative solvent accessibility, relative accessibility (RSA) can be predicted from 3D protein structure using the FreeSASA software indicated by the “_[rsa]_freesasa_het” suffix where rsa specifies either all, backbone, polar, apolar, or residue accessibility; RSA can also be calculated from 3D protein structure using the DSSP software indicated by the “_dssp” suffix. RSA can also be predicted from protein sequence using ACCPro or ACCPro8 tools from the SCRATCH software package indicated by the “_accpro[20]” suffix. The “measurement” can refer to Psi and Phi bond angles of the glycosite residue as “_PHI_” or “_PSI_” respectively. When the “measurement” refers to bond angles, bond angles can be predicted from 3D protein structure using the DSSP software indicated by the “_dssp” suffix. Log OR ranges in the second column of Table 2 and Table 3 are denoted by the first two digest of the largest magnitude number bounding the range. Specifically, −Inf denotes [−Inf,−4.75], −4.7 denotes (−4.75,−4.26], −4.2 denotes (−4.26,−3.72], −3.7 denotes (−3.72,−3.09], −3.0 denotes (−3.09,−2.33], −2.3 denotes (−2.33,−1.28], −1.2 denotes (−1.28,0], 1.2 denotes (0,1.28], 2.3 denotes (1.28,2.33], 3.0 denotes (2.33,3.09], 3.7 denotes (3.09,3.72], 4.2 denotes (3.72,4.26], 4.7 denotes (4.26,4.75], and Inf (4.75, Inf])

TABLE 2
Table of representative IMRs determined by Fisher's exact test

| | Log OR | |
|---|---|---|
| Protein_Structure | Extrema | Glycan_Motifs |
| ASN_seq_aaAll_A | −1.28 | X1184/X1189/X1191/X1193/X1194/X1206/X1207/X1208/X1266/X1316/X1317/X1318/X1320/ |
| | | X1321/X1322/X1852/X1867/X1868/X1869/X187/X1883/X1884/X1894/X192/X193/X194/ |
| | | X195/X1952/X196/X198/X2011/X2012/X2013/X2015/X2021/X2022/X2023/X21/X23/X25/ |
| | | X253/X2734/X2741/X2743/X2756/X2763/X2827/X2895/X2899/X2904/X2910/X2913/X2914/ |
| | | X3802/X394/X3946/X3947/X3953/X3958/X3959/X397/X399/X400/X401/X402/X406/X407/ |
| | | X485/X487/X5106/X705/X714/X715/X717/X718/X719/X727/X728/X729/X73/X75/X76/ |
| | | X78/X822/X823/X824 |
| ASN_seq_aaAll_D | −1.28 | X1/X1164/X1169/X1172/X1355/X1479/X17/X1837/X184/X1840/X2054/X2212/X2713/ |
| | | X385/X393/X68/X696/X703/X853 |
| ASN_seq_aaAll_E | −1.28 | X10425/X10426/X10427/X11095/X1162/X1163/X1165/X1166/X1167/X1170/X1171/X1174/ |
| | | X1177/X1180/X1185/X1186/X16/X183/X1831/X1832/X1833/X1834/X1835/X1836/X1842/ |
| | | X1844/X1847/X1848/X1849/X185/X1853/X1854/X1858/X190/X2699/X2700/X2701/X2702/ |
| | | X2704/X2705/X2707/X2709/X2712/X2716/X2718/X2723/X2724/X2725/X2735/X2736/X3742/ |
| | | X3743/X3744/X3746/X3747/X3749/X3750/X3753/X3755/X3761/X3763/X3768/X3774/X3776/ |
| | | X383/X386/X389/X391/X395/X4915/X4916/X4917/X4919/X4921/X4923/X4924/X4926/X4927/ |
| | | X4929/X4934/X4940/X4942/X6159/X6160/X6161/X6162/X6164/X6165/X6167/X6168/X6170/ |
| | | X6171/X6173/X6174/X6186/X67/X694/X697/X698/X699/X701/X704/X706/X71/X711/X7379/ |
| | | X7380/X7381/X7382/X7384/X7385/X7387/X7389/X7390/X83/X8542/X8543/X8544/X8545/ |
| | | X8547/X8548/X8552/X8553/X9566/X9567/X9568/X9569/X9571 |
| ASN_seq_aaAll_F | −1.28 | X1/X10425/X10426/X10427/X10429/X10459/X11095/X11096/X11581/X1162/X1163/X1164/ |
| | | X1165/X1167/X1168/X1169/X1170/X1171/X1172/X1175/X1176/X1177/X1180/X1181/X1182/ |
| | | X1183/X1185/X1186/X1219/X1220/X1221/X1222/X1223/X1224/X1225/X1226/X1227/X1228/ |
| | | X1229/X1230/X1231/X1232/X1233/X1234/X1235/X1236/X1237/X1238/X1239/X1240/X1431/ |
| | | X1432/X1433/X1435/X1437/X17/X1831/X1832/X1833/X1834/X1835/X1836/X1837/X1839/ |
| | | X184/X1840/X1841/X1842/X1843/X1844/X1848/X1849/X185/X1850/X1851/X1853/X1854/ |
| | | X1856/X1857/X1858/X1860/X1861/X1862/X189/X1895/X1896/X1897/X1898/X1899/X19/ |
| | | X190/X1900/X1901/X1902/X1903/X1904/X1905/X1906/X1907/X1908/X1909/X1910/X1911/ |
| | | X1912/X1913/X1914/X1915/X1916/X1917/X1918/X1919/X1920/X1921/X1922/X1923/X1924/ |
| | | X1925/X2035/X2036/X2145/X2146/X2148/X2149/X215/X2150/X2151/X2152/X2154/X2157/ |
| | | X216/X2160/X217/X218/X26/X2699/X2700/X2701/X2702/X2703/X2704/X2705/X2706/X2707/ |
| | | X2708/X2709/X2712/X2713/X2715/X2716/X2717/X2723/X2724/X2725/X2727/X2728/X2729/ |
| | | X2732/X2733/X2735/X2736/X2738/X2739/X2740/X2764/X2765/X2766/X2767/X2768/X2769/ |
| | | X2770/X2771/X2772/X2773/X2774/X2775/X2776/X2777/X2778/X2779/X2780/X2781/X2782/ |
| | | X2783/X2784/X2785/X2786/X2788/X2789/X2790/X2791/X2792/X2793/X2794/X2795/X2796/ |
| | | X2797/X2798/X2799/X2922/X2923/X2924/X2925/X2926/X2927/X2928/X3/X3032/X3033/X3034/ |
| | | X3035/X3036/X3038/X3039/X3040/X3042/X3044/X3045/X3046/X3050/X3051/X3052/X3053/ |
| | | X3056/X3059/X3060/X3061/X3062/X32/X3742/X3743/X3744/X3745/X3746/X3747/X3749/ |
| | | X3750/X3752/X3753/X3754/X3760/X3761/X3762/X3763/X3766/X3767/X3773/X3774/X3776/ |
| | | X3777/X3779/X3780/X3781/X3782/X3783/X3784/X3787/X3803/X3804/X3805/X3806/X3807/ |
| | | X3808/X3811/X3813/X3814/X3815/X3816/X3817/X3818/X3819/X3820/X3822/X3823/X3824/ |
| | | X3825/X3826/X3827/X3828/X3829/X383/X3830/X3831/X3832/X3833/X3834/X3835/X3836/ |
| | | X385/X387/X388/X389/X391/X395/X396/X3966/X3967/X3968/X3969/X3970/X3972/X3973/ |
| | | X3974/X3975/X3976/X3977/X3978/X4066/X4067/X4069/X4070/X4071/X4072/X4073/X4074/ |
| | | X4075/X4076/X4078/X4079/X4080/X4081/X4082/X4083/X4087/X4088/X4089/X4090/X4093/ |
| | | X4095/X4097/X4098/X4099/X4102/X4103/X4104/X4105/X4108/X4109/X4111/X4112/X4113/ |
| | | X424/X425/X426/X427/X428/X429/X430/X4915/X4916/X4917/X4919/X4920/X4921/X4922/ |
| | | X4924/X4926/X4927/X4928/X4929/X4932/X4933/X4939/X4940/X4941/X4950/X4951/X4952/ |
| | | X4955/X4956/X4957/X4958/X4959/X4971/X4974/X4975/X4976/X4978/X4979/X4980/X4981/ |
| | | X4982/X4983/X4984/X4985/X4987/X4988/X4989/X4990/X4991/X5119/X5120/X5123/X5124/ |
| | | X5125/X5127/X5128/X5129/X5130/X5131/X5132/X5133/X5134/X5135/X5200/X5201/X5202/ |
| | | X5203/X5204/X5205/X5206/X5207/X5208/X5210/X5211/X5212/X5213/X5214/X5215/X5217/ |
| | | X5220/X5221/X5222/X5225/X5227/X5229/X5230/X5231/X5232/X5233/X5237/X5238/X5239/ |
| | | X5240/X5242/X5243/X5244/X5245/X5248/X5249/X5252/X5253/X5254/X5255/X5256/X5257/ |
| | | X5258/X5259/X5260/X532/X6159/X6160/X6161/X6162/X6163/X6165/X6167/X6168/X6171/ |
| | | X6173/X6174/X6175/X6184/X6185/X6193/X6194/X6195/X6196/X6197/X6201/X6202/X6205/ |
| | | X6207/X6208/X6210/X6211/X6325/X6328/X6329/X6330/X6331/X6332/X6334/X6335/X6336/ |
| | | X6337/X6338/X6339/X6381/X6382/X6383/X6384/X6385/X6387/X6389/X6391/X6394/X6395/ |
| | | X6396/X6397/X6399/X6400/X6401/X6403/X6406/X6407/X6408/X6409/X6413/X6414/X6415/ |
| | | X6416/X6418/X6419/X6420/X6422/X6424/X6426/X6427/X6428/X6430/X6431/X6432/X6433/ |
| | | X6435/X6436/X6437/X6438/X6439/X6440/X6442/X6443/X6503/X68/X694/X696/X697/X699/ |
| | | X7/X70/X701/X702/X703/X706/X707/X709/X71/X710/X711/X7379/X7380/X7381/X7382/X7385/ |
| | | X7387/X7388/X7390/X7394/X7395/X7406/X7407/X7412/X744/X745/X746/X747/X748/X749/ |
| | | X750/X7507/X751/X7510/X7511/X7513/X7514/X7515/X7516/X752/X753/X7537/X7538/X754/ |
| | | X7540/X7541/X7544/X7547/X755/X7550/X7551/X7553/X7556/X7557/X7558/X756/X7560/ |
| | | X7563/X7564/X7568/X7570/X7571/X7572/X7573/X7575/X7576/X7577/X7578/X7579/X7580/ |
| | | X7581/X7582/X7584/X7585/X7586/X7587/X7589/X7662/X8542/X8543/X8544/X8545/X8546/ |
| | | X8548/X8553/X8560/X8631/X8632/X8645/X8647/X8654/X8655/X8657/X8660/X8661/X8664/ |
| | | X8665/X8666/X8667/X8668/X8669/X8670/X8673/X8674/X901/X903/X9031/X92/X93/X94/ |
| | | X9566/X9567/X9568/X9569/X9571/X9624/X9630/X9631/X9632/X9634/X9635 |
| ASN_seq_aaAll_G | −1.28 | X1199/X1202/X1204/X1206/X1207/X1876/X1878/X1883/X1951/X197/X198/X24/X25/ |
| | | X2751/X2826/X3864/X403/X406/X407/X722/X725/X727/X728/X729/X77/X78 |
| ASN_seq_aaAll_H | −1.28 | X1184/X1191/X1852/X193/X194/X196/X23/X2734/X3775/X397/X399/X401/X4132/X5278/ |
| | | X5289/X6470/X6478/X714/X717/X75/X76/X7620/X7639/X8714 |
| ASN_seq_aaAll_I | −1.28 | X104/X234/X235/X39/X452/X453/X799 |
| ASN_seq_aaAll_K | −1.28 | X1162/X1163/X1165/X1166/X1167/X1170/X1171/X1174/X1175/X1177/X1180/X1183/X1185/ |
| | | X1206/X1207/X1219/X1222/X1223/X1224/X1225/X1226/X1228/X1229/X1230/X1231/X1232/ |
| | | X1233/X1234/X1235/X1236/X1237/X1238/X1239/X1240/X1431/X1435/X16/X183/X1831/ |
| | | X1832/X1833/X1834/X1835/X1836/X1841/X1842/X1844/X1847/X1848/X1849/X185/X1851/ |
| | | X1853/X1856/X1858/X1883/X1898/X1899/X190/X1901/X1902/X1903/X1904/X1905/X1906/ |
| | | X1907/X1908/X1910/X1911/X1912/X1914/X1915/X1916/X1917/X1918/X1919/X1920/X1921/ |
| | | X1922/X1923/X1924/X1925/X198/X2035/X2146/X2148/X215/X2151/X2154/X216/X217/X218/ |
| | | X25/X26/X2699/X2700/X2701/X2702/X2705/X2706/X2707/X2709/X2712/X2715/X2716/X2718/ |
| | | X2725/X2733/X2735/X2769/X2770/X2771/X2773/X2774/X2775/X2777/X2779/X2780/X2781/ |
| | | X2782/X2783/X2785/X2786/X2789/X2792/X2793/X2794/X2795/X2796/X2797/X2798/X2799/ |
| | | X2922/X2923/X2927/X3036/X3038/X3039/X3042/X3050/X32/X3742/X3744/X3746/X3747/ |
| | | X3749/X3750/X3752/X3753/X3755/X3760/X3761/X3763/X3768/X3815/X3816/X3819/X3820/ |
| | | X3823/X3825/X3826/X3827/X3829/X383/X3832/X3833/X3834/X3835/X3836/X386/X389/ |
| | | X391/X395/X3966/X3970/X3972/X3974/X3976/X406/X407/X4070/X4074/X4079/X4087/X4090/ |
| | | X424/X425/X426/X427/X428/X429/X430/X4915/X4916/X4917/X4919/X4921/X4923/X4924/ |
| | | X4927/X4929/X4934/X4939/X4940/X4942/X4980/X4982/X4984/X4985/X4988/X5120/X5123/ |
| | | X5125/X5127/X5129/X5134/X5211/X5213/X5237/X532/X6/X6159/X6160/X6162/X6164/X6165/ |
| | | X6167/X6168/X6171/X6174/X6186/X6325/X6328/X6334/X6336/X6400/X67/X694/X697/X698/ |
| | | X699/X7/X701/X704/X706/X709/X71/X711/X727/X728/X729/X7379/X7380/X7381/X7382/ |
| | | X7385/X7387/X7389/X7390/X744/X746/X747/X748/X749/X750/X751/X7510/X752/X753/X754/ |
| | | X755/X756/X78/X8542/X8543/X8553/X901/X92/X93/X94/X9567/X9569 |
| ASN_seq_aaAll_M | −1.28 | X1163/X1165/X1171/X16/X1832/X1833/X1834/X1836/X1841/X1842/X1844/X2700/X2701/ |
| | | X2705/X2706/X2707/X2709/X2712/X2715/X2716/X3743/X3749/X3752/X3753/X3760/X3761/ |
| | | X3763/X3766/X383/X4926/X4927/X4932/X4939/X4940/X6173/X6174/X6184/X694/X697/X7394 |
| ASN_seq_aaAll_P | −1.28 | X117/X1194/X13/X1354/X1368/X1415/X1866/X1867/X2125/X263/X2742/X2743/X3935/X45/ |
| | | X466/X508/X509/X5098/X798/X851/X852/X895 |
| ASN_seq_aaAll_Q | −1.28 | X1162/X1163/X1171/X1219/X1222/X1223/X1224/X1225/X1226/X1228/X1229/X1230/X1231/ |
| | | X1232/X1233/X1234/X1235/X1236/X1237/X1238/X1239/X1240/X1431/X1432/X1433/X1435/ |
| | | X1437/X1831/X1834/X1836/X1842/X1844/X1898/X1899/X1901/X1902/X1903/X1904/X1905/ |
| | | X1906/X1907/X1908/X1910/X1911/X1912/X1913/X1914/X1915/X1916/X1917/X1918/X1919/ |
| | | X1920/X1921/X1922/X1923/X1924/X1925/X2035/X2145/X2146/X2148/X2149/X215/X2150/ |
| | | X2151/X2152/X2154/X2157/X216/X217/X218/X26/X2699/X2700/X2702/X2707/X2709/X2712/ |
| | | X2718/X2768/X2769/X2770/X2771/X2773/X2774/X2775/X2776/X2777/X2779/X2780/X2781/ |
| | | X2782/X2783/X2785/X2786/X2789/X2790/X2791/X2792/X2793/X2794/X2795/X2796/X2797/ |
| | | X2798/X2799/X2922/X2923/X2927/X3033/X3034/X3035/X3036/X3038/X3039/X3042/X3044/ |
| | | X3046/X3050/X3051/X3052/X3053/X3059/X3060/X32/X3742/X3743/X3744/X3746/X3749/ |
| | | X3750/X3755/X3761/X3763/X3768/X3805/X3813/X3814/X3815/X3816/X3817/X3819/X3820/ |
| | | X3823/X3825/X3826/X3827/X3828/X3829/X383/X3830/X3831/X3832/X3833/X3834/X3835/ |
| | | X3836/X3966/X3970/X3972/X3974/X3976/X4066/X4067/X4070/X4072/X4073/X4074/X4079/ |
| | | X4080/X4083/X4087/X4088/X4089/X4090/X4095/X4097/X4098/X4099/X4102/X4103/X4105/ |
| | | X4109/X424/X425/X426/X427/X428/X429/X430/X4915/X4916/X4919/X4923/X4924/X4926/ |
| | | X4927/X4929/X4934/X4942/X4976/X4978/X4979/X4980/X4982/X4984/X4985/X4988/X4989/ |
| | | X4990/X4991/X5120/X5123/X5125/X5127/X5129/X5134/X5200/X5201/X5211/X5212/X5213/ |
| | | X5220/X5221/X5227/X5229/X5230/X5231/X5233/X5237/X5238/X5239/X5242/X5249/X5252/ |
| | | X5253/X532/X6/X6160/X6164/X6165/X6167/X6171/X6173/X6174/X6175/X6186/X6207/X6208/ |
| | | X6211/X6325/X6328/X6334/X6336/X6384/X6394/X6395/X6400/X6401/X6406/X6418/X6424/ |
| | | X6426/X6427/X6428/X6433/X697/X7/X7380/X7381/X7382/X7385/X7389/X7390/X744/X746/ |
| | | X747/X748/X749/X750/X751/X7510/X752/X753/X754/X755/X7556/X756/X7563/X7564/X7575/ |
| | | X8542/X8553/X8660/X901/X903/X92/X93/X94/X9569 |
| ASN_seq_aaAll_R | −1.28 | X1/X1162/X1163/X1164/X1165/X1168/X1169/X1170/X1171/X1172/X1175/X1176/X1177/X1179/ |
| | | X1180/X1181/X1182/X1183/X1184/X1185/X1186/X1219/X1222/X1223/X1224/X1225/X1226/ |
| | | X1228/X1229/X1230/X1231/X1232/X1233/X1234/X1235/X1236/X1237/X1238/X1239/X1240/ |
| | | X1356/X1358/X1359/X1361/X1365/X1431/X1432/X1433/X1435/X1437/X17/X1831/X1832/ |
| | | X1833/X1834/X1835/X1836/X1837/X1839/X184/X1840/X1841/X1842/X1843/X1844/X185/ |
| | | X1850/X1851/X1852/X1853/X1854/X1856/X1857/X1858/X1860/X1861/X1862/X187/X189/ |
| | | X1898/X1899/X19/X190/X1901/X1902/X1903/X1904/X1905/X1906/X1907/X1908/X1910/X1911/ |
| | | X1912/X1913/X1914/X1915/X1916/X1917/X1918/X1919/X192/X1920/X1921/X1922/X1923/ |
| | | X1924/X1925/X2035/X2055/X2056/X2057/X2062/X2063/X2066/X2067/X2069/X21/X2145/ |
| | | X2146/X2148/X2149/X215/X2150/X2151/X2152/X2154/X2157/X216/X217/X218/X26/X2699/ |
| | | X2700/X2701/X2702/X2703/X2705/X2706/X2707/X2708/X2709/X2712/X2713/X2715/X2716/ |
| | | X2717/X2718/X2723/X2727/X2728/X2729/X2732/X2733/X2734/X2735/X2736/X2738/X2739/ |
| | | X2740/X2768/X2769/X2770/X2771/X2773/X2774/X2775/X2776/X2777/X2779/X2780/X2781/ |
| | | X2782/X2783/X2785/X2786/X2788/X2789/X2790/X2791/X2792/X2793/X2794/X2795/X2796/ |
| | | X2797/X2798/X2799/X2922/X2923/X2924/X2927/X2944/X2946/X2950/X2951/X2952/X2953/ |
| | | X2955/X2956/X2957/X2958/X2960/X2962/X3/X3032/X3033/X3034/X3035/X3036/X3038/X3039/ |
| | | X3040/X3042/X3044/X3045/X3046/X3050/X3051/X3052/X3053/X3059/X3060/X3062/X32/ |
| | | X3742/X3744/X3745/X3746/X3747/X3749/X3750/X3752/X3753/X3754/X3755/X3760/X3761/ |
| | | X3762/X3763/X3766/X3767/X3768/X3769/X3774/X3776/X3777/X3779/X3780/X3781/X3782/ |
| | | X3783/X3784/X3787/X3805/X3811/X3813/X3814/X3815/X3816/X3817/X3819/X3820/X3822/ |
| | | X3823/X3825/X3826/X3827/X3828/X3829/X383/X3830/X3831/X3832/X3833/X3834/X3835/ |
| | | X3836/X385/X387/X388/X389/X390/X391/X392/X394/X395/X396/X3966/X3968/X3970/X3972/ |
| | | X3973/X3974/X3976/X3992/X3993/X3994/X3995/X3996/X3997/X3998/X4000/X4001/X4002/ |
| | | X4005/X4006/X4008/X4009/X4066/X4067/X4069/X4070/X4071/X4072/X4073/X4074/X4076/ |
| | | X4078/X4079/X4080/X4082/X4083/X4087/X4088/X4089/X4090/X4095/X4097/X4098/X4099/ |
| | | X4102/X4103/X4105/X4109/X4111/X4112/X424/X425/X426/X427/X428/X429/X430/X4915/ |
| | | X4916/X4917/X4919/X4920/X4921/X4922/X4923/X4924/X4927/X4928/X4929/X4932/X4933/ |
| | | X4934/X4935/X4939/X4940/X4941/X4942/X4947/X4948/X4951/X4952/X4955/X4956/X4957/ |
| | | X4958/X4959/X4974/X4976/X4978/X4979/X4980/X4982/X4984/X4985/X4987/X4988/X4989/ |
| | | X4990/X4991/X511/X5120/X5123/X5125/X5127/X5128/X5129/X5131/X5134/X5141/X5142/ |
| | | X5143/X5144/X5145/X5147/X5150/X5151/X5152/X5153/X5154/X5155/X5200/X5201/X5203/ |
| | | X5206/X5208/X5210/X5211/X5212/X5213/X5214/X5220/X5221/X5222/X5227/X5229/X5230/ |
| | | X5231/X5232/X5233/X5237/X5238/X5239/X5242/X5245/X5249/X5252/X5253/X5255/X5259/ |
| | | X532/X6159/X6160/X6162/X6163/X6164/X6165/X6167/X6168/X6170/X6174/X6175/X6176/ |
| | | X6181/X6182/X6184/X6185/X6186/X6187/X6191/X6194/X6196/X6197/X6205/X6207/X6208/ |
| | | X6211/X6325/X6328/X6334/X6335/X6336/X6338/X6344/X6346/X6347/X6348/X6349/X6350/ |
| | | X6351/X6352/X6383/X6384/X6394/X6395/X6397/X6399/X6400/X6401/X6406/X6409/X6413/ |
| | | X6418/X6424/X6426/X6427/X6428/X6433/X6438/X68/X694/X696/X697/X699/X7/X70/X700/ |
| | | X701/X702/X703/X705/X706/X707/X709/X71/X710/X711/X73/X7379/X7380/X7381/X7384/ |
| | | X7387/X7388/X7389/X7390/X7396/X7397/X7401/X7403/X7404/X7407/X744/X746/X747/X748/ |
| | | X749/X750/X751/X7510/X7513/X7515/X7517/X7518/X7519/X752/X7520/X753/X754/X7541/ |
| | | X755/X7550/X7556/X756/X7563/X7564/X7575/X854/X8542/X8543/X8547/X855/X8552/X8555/ |
| | | X8556/X8558/X858/X8631/X8660/X8716/X901/X903/X92/X93/X94/X9569 |
| ASN_seq_aaAll_S | −1.28 | X1164/X1169/X117/X1172/X1179/X1184/X13/X1354/X1355/X1479/X1837/X184/X1840/X1843/ |
| | | X1852/X1857/X189/X2054/X2212/X263/X2708/X2713/X2734/X3762/X385/X388/X394/X45/ |
| | | X508/X509/X696/X70/X700/X703/X705/X851/X852/X853 |
| ASN_seq_aaAll_T | −1.28 | X1174/X1206/X1207/X1208/X1266/X1267/X16/X183/X1849/X1883/X1884/X1951/X1952/X1953/ |
| | | X198/X25/X2756/X2826/X2827/X2828/X386/X3864/X3865/X3947/X406/X407/X49/X6/X67/ |
| | | X698/X704/X727/X728/X729/X78 |
| ASN_seq_aaAll_V | −1.28 | X1162/X1163/X1164/X1165/X1167/X1169/X1171/X1172/X1174/X1177/X1180/X1181/X1206/ |
| | | X1207/X1266/X17/X1831/X1833/X1834/X1836/X1837/X184/X1840/X1841/X1842/X1843/X1844/ |
| | | X1848/X1849/X1858/X1883/X190/X1952/X198/X25/X2699/X2701/X2702/X2703/X2706/X2707/ |
| | | X2708/X2709/X2712/X2713/X2715/X2716/X2725/X2827/X3742/X3744/X3745/X3746/X3747/ |
| | | X3752/X3753/X3760/X3761/X3762/X3763/X383/X385/X389/X3947/X395/X396/X406/X407/ |
| | | X4916/X4917/X4919/X4920/X4922/X4939/X4940/X6/X6163/X6167/X6168/X694/X696/X697/ |
| | | X698/X701/X703/X706/X707/X71/X711/X727/X728/X729/X7388/X78 |
| ASN_seq_aaDown_A | −1.28 | X1184/X1852/X2734 |
| ASN_seq_aaDown_C | −1.28 | X1206/X1207/X1266/X1883/X1952/X198/X25/X2827/X406/X407/X727/X728X729/X78 |
| ASN_seq_aaDown_D | −1.28 | X393 |
| ASN_seq_aaDown_E | −1.28 | X1163/X1165/X1166/X1167/X1168/X1170/X1171/X1174/X1175/X1177/X1180/X1182/X1183/ |
| | | X1185/X1186/X16/X183/X1833/X1834/X1835/X1836/X1839/X1842/X1844/X1847/X1848/X1849/ |
| | | X185/X1850/X1851/X1853/X1854/X1856/X1858/X1860/X1861/X1862/X190/X2700/X2701/ |
| | | X2704/X2707/X2709/X2712/X2716/X2718/X2723/X2724/X2725/X2727/X2728/X2729/X2732/ |
| | | X2733/X2735/X2736/X2738/X2739/X2740/X3743/X3749/X3753/X3755/X3761/X3763/X3768/ |
| | | X3769/X3773/X3774/X3775/X3776/X3777/X3779/X3780/X3781/X3782/X3783/X3784/X3787/ |
| | | X383/X386/X387/X389/X391/X395/X4915/X4923/X4926/X4927/X4929/X4934/X4935/X4940/ |
| | | X4942/X4947/X4948/X4950/X4951/X4952/X4955/X4956/X4957/X4958/X4959/X6160/X6164/ |
| | | X6170/X6173/X6174/X6175/X6176/X6181/X6182/X6186/X6187/X6191/X6193/X6194/X6195/ |
| | | X6196/X6197/X67/X694/X697/X698/X699/X701/X702/X704/X706/X709/X71/X711/X7381/ |
| | | X7384/X7389/X7394/X7395/X7396/X7397/X7401/X7403/X7404/X7406/X7407/X8545/X8547/ |
| | | X8552/X8555/X8556/X8558 |
| ASN_seq_aaDown_F | −1.28 | X1164/X1166/X1167/X1168/X1169/X1170/X1172/X1174/X1175/X1176/X1177/X1180/X1181/ |
| | | X1182/X1183/X1185/X16/X17/X183/X1835/X1837/X1839/X184/X1840/X1843/X1847/X1848/ |
| | | X1849/X185/X1850/X1851/X1853/X1854/X1856/X1857/X1858/X1860/X1861/X1862/X187/X189/ |
| | | X19/X190/X192/X21/X2703/X2704/X2708/X2713/X2723/X2724/X2725/X2727/X2728/X2729/ |
| | | X2732/X2733/X2735/X2736/X2738/X2739/X2740/X3/X3745/X3747/X3762/X3766/X3767/X3773/ |
| | | X3774/X3775/X3776/X3777/X3779/X3780/X3781/X3782/X3783/X3784/X3787/X385/X386/X387/ |
| | | X388/X389/X391/X395/X396/X49/X4917/X4920/X4921/X4922/X4928/X4932/X4933/X4950/ |
| | | X4951/X4952/X4955/X4956/X4957/X4958/X4959/X6/X6159/X6161/X6162/X6163/X6168/X6184/ |
| | | X6185/X6193/X6194/X6195/X6196/X6197/X67/X68/X696/X698/X699/X70/X700/X701/X702/ |
| | | X703/X706/X707/X709/X71/X710/X711/X73/X7379/X7387/X7388/X7406/X7407/X8543/X8560 |
| ASN_seq_aaDown_G | −1.28 | X117/X1206/X1207/X13/X1354/X1883/X198/X25/X263/X406/X407/X45/X508/X509/X6/X708/ |
| | | X727/X728/X729/X74/X78/X851/X852 |
| ASN_seq_aaDown_H | −1.28 | X8560 |
| ASN_seq_aaDown_I | −1.28 | X1207/X1266/X1883/X190/X1952/X2827/X389/X6/X69/X71/X729 |
| ASN_seq_aaDown_K | −1.28 | X1163/X1164/X1169/X1171/X1172/X1174/X1832/X1834/X1836/X1837/X184/X1840/X1842/ |
| | | X187/X189/X192/X21/X2700/X2705/X2707/X2712/X2713/X3749/X3750/X3761/X383/X385/ |
| | | X388/X390/X392/X396/X4927/X4928/X4929/X696/X697/X698/X70/X700/X703/X707/X708/X73 |
| ASN_seq_aaDown_L | −1.28 | X117/X1177/X1180/X13/X1354/X1357/X1360/X1858/X187/X190/X192/X2058/X2059/X2061/ |
| | | X2064/X21/X214/X263/X2945/X2949/X2954/X2959/X389/X390/X392/X395/X3991/X4004/ |
| | | X4007/X45/X508/X509/X510/X5146/X5148/X701/X706/X708/X71/X73/X84/X851/X852/X856 |
| ASN_seq_aaDown_M | −1.28 | X1/X1162/X1164/X1165/X1169/X1172/X1176/X1186/X17/X1831/X1832/X1833/X1837/X184/ |
| | | X1840/X1841/X1844/X1854/X1857/X189/X19/X2699/X2701/X2702/X2705/X2706/X2709/X2713/ |
| | | X2715/X2716/X2717/X3/X3742/X3744/X3746/X3750/X3752/X3753/X3754/X3760/X3763/X3766/ |
| | | X385/X388/X4916/X4919/X4921/X4928/X4932/X4939/X4940/X4941/X6162/X6167/X6184/X68/ |
| | | X694/X696/X70/X703/X710/X7387 |
| ASN_seq_aaDown_P | −1.28 | X1199/X1202/X1876/X197/X403/X453/X49/X722/X725 |
| ASN_seq_aaDown_Q | −1.28 | X1/X1162/X1163/X1164/X1169/X1171/X1172/X17/X1831/X1834/X1836/X1837/X184/X1840/ |
| | | X1842/X2699/X2700/X2702/X2707/X2712/X2713/X2718/X3742/X3744/X3746/X3749/X3755/ |
| | | X3761/X383/X385/X4916/X4919/X4923/X4924/X4927/X4942/X6/X6164/X6165/X6167/X68/ |
| | | X696/X697/X703/X7380/X7389/X7390/X8542/X9569 |
| ASN_seq_aaDown_R | −1.28 | X1/X1162/X1163/X1164/X1165/X1168/X1169/X1170/X1171/X1172/X1175/X1176/X1177/ |
| | | X1179/X1180/X1182/X1183/X1184/X1185/X1186/X17/X1831/X1832/X1833/X1835/X1836/ |
| | | X1837/X1839/X184/X1840/X1841/X1842/X1843/X1848/X185/X1850/X1851/X1852/X1853/ |
| | | X1854/X1856/X1857/X1858/X1860/X1861/X1862/X189/X19/X190/X2699/X2700/X2701/X2702/ |
| | | X2703/X2704/X2705/X2706/X2707/X2708/X2713/X2715/X2716/X2717/X2723/X2725/X2727/ |
| | | X2728/X2729/X2732/X2733/X2734/X2735/X2736/X2738/X3/X3742/X3743/X3744/X3745/X3746/ |
| | | X3747/X3749/X3750/X3752/X3753/X3754/X3760/X3762/X3766/X3767/X3769/X3773/X3774/ |
| | | X3776/X3779/X3780/X3782/X3783/X3784/X383/X385/X387/X388/X389/X391/X394/X395/ |
| | | X4915/X4916/X4917/X4919/X4920/X4921/X4922/X4926/X4927/X4928/X4929/X4932/X4933/ |
| | | X4935/X4939/X4940/X4941/X4947/X4948/X4950/X4951/X4952/X4956/X4957/X4958/X6159/ |
| | | X6160/X6162/X6163/X6167/X6168/X6170/X6173/X6174/X6175/X6181/X6182/X6184/X6185/ |
| | | X6197/X68/X694/X696/X697/X699/X70/X700/X701/X702/X703/X705/X706/X709/X71/X710/ |
| | | X711/X7379/X7380/X7381/X7382/X7384/X7387/X7388/X7390/X7396/X7403/X7404/X8542/ |
| | | X8543/X8545/X8547/X8552/X9569 |
| ASN_seq_aaDown_S | −1.28 | X1164/X1169/X1172/X1176/X1179/X1184/X1837/X184/X1840/X1843/X1852/X1857/X187/X189/ |
| | | X19/X192/X21/X2708/X2713/X2734/X2738/X3762/X3779/X385/X388/X394/X4956/X68/X696/ |
| | | X70/X700/X703/X705/X710/X73 |
| ASN_seq_aaDown_T | −1.28 | X1199/X1202/X1204/X1206/X1207/X1208/X16/X183/X1849/X1876/X1878/X1883/X1884/X197/ |
| | | X198/X2017/X24/X25/X2751/X2756/X2901/X386/X3947/X3955/X403/X406/X407/X49/X5108/ |
| | | X6/X67/X698/X704/X722/X725/X727/X728/X729/X77/X78 |
| ASN_seq_aaDown_V | −1.28 | X1/X1162/X1163/X1164/X1165/X1166/X1167/X1169/X1170/X1171/X1172/X1174/X1175/ |
| | | X1176/X1177/X1180/X1181/X1183/X1185/X1186/X1219/X1222/X1223/X1224/X1225/X1226/ |
| | | X1228/X1229/X1230/X1231/X1232/X1233/X1234/X1235/X1236/X1237/X1238/X1239/X1240/ |
| | | X1431/X1432/X1433/X1435/X1437/X17/X183/X1831/X1832/X1833/X1834/X1835/X1836/X1837/ |
| | | X184/X1840/X1841/X1842/X1843/X1844/X1847/X1848/X1849/X185/X1851/X1853/X1854/ |
| | | X1856/X1857/X1858/X1861/X187/X189/X1898/X1899/X190/X1901/X1902/X1903/X1904/X1905/ |
| | | X1906/X1907/X1908/X1910/X1911/X1912/X1913/X1914/X1915/X1916/X1917/X1918/X1919/ |
| | | X192/X1920/X1921/X1922/X1923/X1924/X1925/X2035/X21/X2145/X2146/X2148/X2149/X215/ |
| | | X2150/X2151/X2152/X2154/X2157/X216/X217/X218/X26/X2699/X2700/X2701/X2702/X2703/ |
| | | X2704/X2705/X2706/X2707/X2708/X2709/X2712/X2713/X2715/X2716/X2717/X2718/X2723/ |
| | | X2724/X2725/X2728/X2733/X2735/X2736/X2768/X2769/X2770/X2771/X2773/X2774/X2775/ |
| | | X2776/X2777/X2779/X2780/X2781/X2782/X2783/X2785/X2786/X2789/X2790/X2791/X2792/ |
| | | X2793/X2794/X2795/X2796/X2797/X2798/X2799/X2922/X2923/X2927/X3033/X3034/X3035/ |
| | | X3036/X3038/X3039/X3042/X3044/X3046/X3050/X3051/X3052/X3053/X3059/X3060/X32/ |
| | | X3742/X3743/X3744/X3745/X3746/X3747/X3749/X3750/X3752/X3753/X3754/X3755/X3760/ |
| | | X3761/X3762/X3763/X3766/X3767/X3768/X3773/X3774/X3775/X3776/X3777/X3784/X3805/ |
| | | X3813/X3814/X3815/X3816/X3817/X3819/X3820/X3823/X3825/X3826/X3827/X3828/X3829/ |
| | | X383/X3830/X3831/X3832/X3833/X3834/X3835/X3836/X385/X388/X389/X390/X391/X392/ |
| | | X395/X396/X3966/X3970/X3972/X3974/X3976/X4066/X4067/X4070/X4072/X4073/X4074/ |
| | | X4079/X4080/X4083/X4087/X4088/X4089/X4090/X4095/X4097/X4098/X4099/X4102/X4103/ |
| | | X4105/X4109/X424/X425/X426/X427/X428/X429/X430/X4915/X4916/X4917/X4919/X4920/ |
| | | X4921/X4922/X4923/X4924/X4926/X4927/X4928/X4929/X4932/X4933/X4934/X4939/X4940/ |
| | | X4941/X4942/X4950/X4951/X4952/X4976/X4978/X4979/X4980/X4982/X4984/X4985/X4988/ |
| | | X4989/X4990/X4991/X5120/X5123/X5125/X5127/X5129/X5134/X5200/X5201/X5211/X5212/ |
| | | X5213/X5220/X5221/X5227/X5229/X5230/X5231/X5233/X5237/X5238/X5239/X5242/X5249/ |
| | | X5252/X5253/X532/X6/X6159/X6160/X6161/X6162/X6163/X6164/X6165/X6167/X6168/X6171/ |
| | | X6173/X6174/X6175/X6184/X6185/X6186/X6193/X6207/X6208/X6211/X6325/X6328/X6334/ |
| | | X6336/X6384/X6394/X6395/X6400/X6401/X6406/X6418/X6424/X6426/X6427/X6428/X6433/ |
| | | X68/X69/X694/X696/X697/X698/X699/X7/X70/X700/X701/X703/X706/X707/X708/X709/X71/ |
| | | X710/X711/X73/X7379/X7380/X7381/X7382/X7385/X7387/X7388/X7389/X7390/X7394/X744/ |
| | | X746/X747/X748/X749/X750/X751/X7510/X752/X753/X754/X755/X7556/X756/X7563/X7564/ |
| | | X7575/X8542/X8543/X8545/X8546/X8553/X8660/X901/X903/X92/X93/X94/X9567/X9569 |
| ASN_seq_aaDown_Y | −1.28 | X1/X1163/X1164/X1169/X1171/X1172/X1176/X1179/X17/X1832/X1834/X1836/X1837/X184/ |
| | | X1840/X1842/X1843/X1857/X189/X19/X2705/X2707/X2708/X2712/X2713/X3/X3750/X3761/ |
| | | X3762/X383/X385/X388/X393/X4923/X6164/X6186/X68/X696/X697/X70/X700/X703/X7389 |
| ASN_seq_aaUp_A | −1.28 | X105/X1189/X1194/X1206/X1207/X1208/X1283/X1284/X1867/X187/X1883/X1884/X192/X198/ |
| | | X1980/X21/X241/X25/X2743/X2756/X392/X400/X406/X407/X461/X464/X69/X718/X727/X728/ |
| | | X729/X73/X78/X789/X792/X795 |
| ASN_seq_aaUp_C | −1.28 | X1176/X1179/X1184/X1852/X1857/X187/X189/X192/X21/X2734/X388/X390/X392/X394/X69/ |
| | | X70/X700/X705/X710/X73/X74 |
| ASN_seq_aaUp_D | −1.28 | X117/X13/X1354/X263/X45/X508/X509/X851/X852 |
| ASN_seq_aaUp_E | −1.28 | X10425/X11095/X1162/X1163/X1171/X1172/X17/X1831/X1832/X1834/X1836/X1837/X1842/ |
| | | X2699/X2702/X2705/X2707/X2712/X2713/X2718/X3742/X3744/X3746/X3750/X3755/X3761/ |
| | | X3763/X383/X4916/X4919/X4923/X4924/X4942/X6161/X6164/X6165/X6167/X6171/X68/X697/ |
| | | X7380/X7385/X7389/X7390/X8542/X8553/X9566/X9569 |
| ASN_seq_aaUp_F | −1.28 | X1162/X1163/X1165/X1167/X1168/X1170/X1171/X1175/X1177/X1180/X1182/X1183/X1185/ |
| | | X1186/X1831/X1833/X1834/X1835/X1836/X1839/X1841/X1842/X1844/X1848/X185/X1850/ |
| | | X1851/X1853/X1854/X1856/X1858/X1860/X1861/X1862/X188/X190/X2699/X2700/X2701/ |
| | | X2702/X2704/X2706/X2707/X2709/X2712/X2715/X2716/X2717/X2718/X2723/X2725/X2727/ |
| | | X2728/X2729/X2732/X2733/X2735/X2736/X2738/X2739/X2740/X3742/X3743/X3744/X3746/ |
| | | X3747/X3749/X3752/X3753/X3754/X3755/X3760/X3761/X3763/X3766/X3767/X3768/X3769/ |
| | | X3773/X3774/X3776/X3779/X3780/X3781/X3782/X3783/X3784/X3787/X383/X387/X389/X391/ |
| | | X395/X4915/X4916/X4917/X4919/X4921/X4922/X4923/X4926/X4927/X4929/X4932/X4933/ |
| | | X4934/X4935/X4939/X4940/X4941/X4942/X4947/X4948/X4950/X4951/X4952/X4955/X4956/ |
| | | X4957/X4958/X4959/X6160/X6162/X6163/X6164/X6167/X6168/X6170/X6173/X6174/X6175/ |
| | | X6176/X6181/X6182/X6184/X6185/X6186/X6187/X6191/X6193/X6194/X6195/X6196/X6197/ |
| | | X694/X697/X699/X701/X702/X706/X709/X71/X711/X7381/X7382/X7384/X7387/X7388/X7389/ |
| | | X7394/X7395/X7396/X7397/X7401/X7403/X7404/X7407/X8545/X8546/X8552/X8555/X8556/ |
| | | X8558/X9575 |
| ASN_seq_aaUp_G | −1.28 | X1168/X1182/X1186/X1357/X1360/X1839/X1850/X1860/X1862/X2058/X2059/X2061/X2064/ |
| | | X214/X2704/X2727/X2729/X2732/X2738/X2739/X2740/X2945/X2949/X2954/X2959/X3773/ |
| | | X3779/X3780/X3781/X3782/X3787/X387/X3991/X4004/X4007/X49/X4950/X4951/X4955/ |
| | | X4956/X4957/X4958/X4959/X510/X5146/X5148/X6193/X6194/X6195/X6196/X6197/X702/ |
| | | X7406/X7407/X84/X856/X8560/X9575 |
| ASN_seq_aaUp_H | −1.28 | X393/X708 |
| ASN_seq_aaUp_I | −1.28 | X1166/X1168/X1176/X1179/X1181/X1182/X1184/X1847/X1850/X1852/X1857/X187/X189/X19/ |
| | | X192/X21/X2724/X2732/X2734/X3/X3775/X3780/X3781/X3782/X388/X390/X392/X394/X396/ |
| | | X4957/X4959/X6196/X6197/X70/X700/X702/X705/X707/X708/X710/X73/X7407 |
| ASN_seq_aaUp_K | −1.28 | X1163/X1167/X1170/X1171/X1174/X1175/X1177/X1180/X1183/X1185/X1206/X1207/X1223/ |
| | | X1224/X1225/X1226/X1231/X1240/X16/X183/X1834/X1836/X1842/X1849/X185/X1851/X1853/ |
| | | X1856/X1858/X1883/X190/X1904/X1907/X1908/X1914/X1919/X198/X2035/X216/X217/X25/ |
| | | X26/X2707/X2712/X2733/X2777/X2782/X2799/X2923/X3761/X3829/X383/X386/X389/X391/ |
| | | X395/X3970/X406/X407/X424/X426/X427/X49/X6/X67/X697/X698/X699/X7/X701/X704/X706/ |
| | | X709/X71/X711/X727/X728/X729/X744/X748/X749/X751/X752/X78/X92 |
| ASN_seq_aaUp_L | −1.28 | X1206/X1207/X1208/X1219/X1222/X1223/X1224/X1225/X1226/X1228/X1229/ |
| | | X1230/X1231/X1232/X1233/X1234/X1235/X1236/X1237/X1238/X1239/X1240/ |
| | | X1266/X1431/X1435/X1883/X1884/X1898/X1899/X1901/X1902/X1903/ |
| | | X1904/X1905/X1906/X1907/X1908/X1910/X1911/X1912/X1914/X1915/X1916/ |
| | | X1917/X1918/X1919/X1920/X1921/X1922/X1923/X1924/X1925/X1952/X198/ |
| | | X2035/X2146/X2148/X215/X2151/X2154/X216/X217/X218/X25/X26/X2756/ |
| | | X2769/X2770/X2771/X2773/X2774/X2775/X2777/X2779/X2780/X2781/X2782/ |
| | | X2783/X2785/X2786/X2789/X2792/X2793/X2794/X2795/X2796/X2797/ |
| | | X2798/X2799/X2827/X2922/X2923/X2927/X3036/X3038/X3039/X3042/X3050/ |
| | | X32/X3815/X3816/X3819/X3820/X3823/X3825/X3826/X3827/X3829/X3832/ |
| | | X3833/X3834/X3835/X3836/X3966/X3970/X3972/X3974/X3976/X406/X407/ |
| | | X4070/X4074/X4079/X4087/X4090/X424/X425/X426/X427/X428/X429/X430/ |
| | | X4980/X4982/X4984/X4985/X4988/X5120/X5123/X5125/X5127/X5129/ |
| | | X5134/X5211/X5213/X5237/X532/X6/X6325/X6328/X6334/X6336/X6400/X7/ |
| | | X727/X728/X729/X744/X746/X747/X748/X749/X750/X751/X7510/X752/X753/ |
| | | X754/X755/X756/X78/X901/X92/X93/X94 |
| ASN_seq_aaUp_P | −1.28 | X1191/X1193/X1357/X1360/X1368/X1869/X193/X194/X195/X196/X2058/X2059/ |
| | | X2061/X214/X23/X2945/X2949/X2954/X397/X399/X3991/X4004/X401/ |
| | | X402/X466/X510/X5146/X714/X715/X717/X719/X75/X76/X798/X84/X856 |
| ASN_seq_aaUp_Q | −1.28 | X1207/X1266/X16/X183/X1883/X1952/X2827/X386/X6/X67/X704/X729 |
| ASN_seq_aaUp_R | −1.28 | X1164/X1169/X1172/X1176/X1177/X1183/X1185/X1186/X1837/X184/X1840/ |
| | | X1843/X1844/X185/X1851/X1853/X1854/X1857/X1862/X189/X190/X193/X194/ |
| | | X196/X23/X2703/X2708/X2709/X2713/X2718/X2729/X2735/X2739/X2740/ |
| | | X3745/X3755/X3762/X3768/X3780/X3781/X3784/X3787/X385/X388/X389/ |
| | | X391/X394/X395/X396/X397/X399/X401/X4920/X4934/X4942/X4948/X4955/ |
| | | X4959/X6182/X6196/X6197/X696/X699/X70/X700/X703/X706/X707/X71/ |
| | | X710/X711/X714/X7404/X7407/X75/X76 |
| ASN_seq_aaUp_S | −1.28 | X117/X13/X1354/X188/X263/X393/X45/X508/X509/X851/X852 |
| ASN_seq_aaUp_T | −1.28 | X1167/X1168/X1170/X1175/X1176/X1177/X1180/X1182/X1183/X1184/X1185/ |
| | | X1835/X1839/X185/X1850/X1851/X1852/X1853/X1856/X1857/X1858/X1860/ |
| | | X1861/X19/X190/X2725/X2727/X2728/X2732/X2733/X2734/X2735/X2738/ |
| | | X2739/X2740/X3/X3773/X3774/X3776/X3779/X3781/X3782/X3783/X3787/ |
| | | X387/X389/X391/X395/X49/X4950/X4951/X4955/X4956/X4958/X4959/X6191/ |
| | | X6193/X6195/X6196/X6197/X699/X701/X702/X706/X709/X71/X710/X711/ |
| | | X7395/X7401/X7406/X7407/X8558/X8560/X9575 |
| ASN_seq_aaUp_V | −1.28 | X1206/X1207/X1843/X1858/X1883/X198/X25/X2708/X393/X3947/X406/X407/ |
| | | X6/X727/X728/X729/X78 |
| ASN_seq_aaUp_W | −1.28 | X1168/X1182/X1184/X1839/X1850/X1852/X1860/X1861/X2727/X2728/X2732/ |
| | | X2734/X2738/X3779/X3782/X387/X4956/X702 |
| ASN_seq_RSA_accproe | −1.28 | X1/X10425/X10426/X10454/X10457/X10458/X10459/X10460/X10461/X10656/ |
| | | X10670/X10679/X10695/X10700/X10702/X11095/X11249/X11422/X1162/ |
| | | X1163/X1164/X1165/X1169/X1170/X1171/X1172/X1177/X1180/X1220/X1221/ |
| | | X1222/X1227/X1229/X1230/X1232/X1234/X1235/X1236/X1238/X1239/X1431/ |
| | | X1432/X1433/X1435/X1437/X1464/X1557/X17/X1831/X1832/X1833/X1834/ |
| | | X1836/X1837/X184/X1840/X1841/X1842/X1843/X1844/X185/X1858/X1895/ |
| | | X1896/X1897/X190/X1900/X1902/X1903/X1905/X1906/X1909/X1911/X1912/ |
| | | X1913/X1915/X1917/X1918/X1921/X1922/X1924/X1925/X2036/X2145/ |
| | | X2146/X2148/X2149/X215/X2150/X2151/X2152/X2154/X2157/X2160/X218/ |
| | | X2186/X2211/X2331/X2332/X2699/X2700/X2701/X2702/X2703/X2705/X2706/ |
| | | X2707/X2708/X2709/X2712/X2713/X2715/X2716/X2717/X2718/X2735/X2736/ |
| | | X2764/X2765/X2766/X2767/X2768/X2769/X2772/X2774/X2775/X2776/ |
| | | X2778/X2780/X2781/X2783/X2784/X2786/X2787/X2788/X2789/X2790/X2791/ |
| | | X2792/X2794/X2795/X2797/X2798/X2924/X2925/X2926/X2927/X2928/X3032/ |
| | | X3033/X3034/X3035/X3036/X3038/X3039/X3040/X3042/X3044/X3045/ |
| | | X3046/X3050/X3051/X3052/X3053/X3056/X3059/X3060/X3061/X3062/X3115/ |
| | | X3117/X32/X3285/X3286/X3287/X3293/X3294/X3299/X3332/X3742/X3743/ |
| | | X3744/X3745/X3746/X3747/X3749/X3750/X3752/X3753/X3754/X3755/X3760/ |
| | | X3761/X3762/X3763/X3768/X3769/X3803/X3804/X3805/X3806/X3807/ |
| | | X3808/X3809/X3810/X3811/X3812/X3813/X3814/X3815/X3817/X3818/X3820/ |
| | | X3821/X3822/X3823/X3824/X3826/X3827/X3828/X383/X3830/X3831/X3832/ |
| | | X3835/X3836/X385/X389/X391/X3967/X3968/X3969/X3972/X3973/X3974/ |
| | | X3975/X3976/X3977/X3978/X4066/X4067/X4069/X4070/X4071/X4072/X4073/ |
| | | X4074/X4075/X4076/X4078/X4079/X4080/X4081/X4082/X4083/X4087/ |
| | | X4088/X4089/X4090/X4093/X4095/X4096/X4097/X4098/X4099/X4102/X4103/ |
| | | X4104/X4105/X4108/X4109/X4110/X4111/X4112/X4113/X4158/X4166/X425/ |
| | | X429/X430/X4373/X4374/X4376/X4378/X4380/X4384/X4385/X4389/X4391/ |
| | | X4392/X4394/X4397/X4442/X4915/X4916/X4917/X4919/X4920/X4921/ |
| | | X4923/X4924/X4926/X4927/X4929/X4934/X4935/X4939/X4940/X4941/X4942/ |
| | | X4968/X4969/X4970/X4971/X4972/X4973/X4974/X4975/X4976/X4977/X4978/ |
| | | X4979/X4980/X4981/X4983/X4985/X4986/X4987/X4988/X4989/X4990/ |
| | | X4991/X5119/X5123/X5124/X5125/X5127/X5128/X5129/X5130/X5131/X5132/ |
| | | X5133/X5134/X5135/X5200/X5201/X5202/X5203/X5204/X5205/X5206/X5207/ |
| | | X5208/X5210/X5211/X5212/X5213/X5214/X5215/X5217/X5218/X5219/ |
| | | X5220/X5221/X5222/X5225/X5227/X5228/X5229/X5230/X5231/X5232/X5233/ |
| | | X5237/X5238/X5239/X5240/X5241/X5242/X5243/X5244/X5245/X5248/X5249/ |
| | | X5250/X5252/X5253/X5254/X5255/X5256/X5257/X5258/X5259/X5260/ |
| | | X5309/X5310/X5315/X532/X5564/X5565/X5566/X5567/X5568/X5570/X5572/ |
| | | X5575/X5577/X5579/X5580/X5583/X5585/X5586/X5588/X5590/X5591/X5594/ |
| | | X5596/X5598/X5604/X5659/X5972/X6/X6159/X6160/X6161/X6162/X6164/ |
| | | X6165/X6167/X6168/X6170/X6171/X6173/X6174/X6175/X6176/X6186/X6187/ |
| | | X6199/X6200/X6201/X6202/X6203/X6204/X6205/X6206/X6207/X6208/ |
| | | X6209/X6210/X6211/X6325/X6328/X6329/X6330/X6331/X6332/X6334/X6335/ |
| | | X6336/X6337/X6338/X6339/X6381/X6382/X6383/X6384/X6385/X6386/X6387/ |
| | | X6388/X6389/X6391/X6392/X6393/X6394/X6395/X6396/X6397/X6399/ |
| | | X6400/X6401/X6402/X6403/X6404/X6405/X6406/X6407/X6408/X6409/X6413/ |
| | | X6414/X6415/X6416/X6417/X6418/X6419/X6420/X6422/X6424/X6425/X6426/ |
| | | X6427/X6428/X6430/X6431/X6432/X6433/X6434/X6435/X6436/X6437/ |
| | | X6438/X6439/X6440/X6441/X6442/X6443/X6503/X6506/X6507/X6510/X6760/ |
| | | X6761/X6762/X6763/X6764/X6765/X6767/X6769/X6771/X6772/X6773/X6775/ |
| | | X6777/X6780/X6782/X6784/X6787/X6789/X6791/X6793/X6795/X6796/ |
| | | X6798/X68/X6805/X6807/X6812/X694/X696/X697/X699/X701/X703/X706/X708/ |
| | | X71/X711/X7379/X7380/X7381/X7382/X7384/X7385/X7387/X7389/X7390/ |
| | | X7396/X7408/X7409/X7411/X7412/X745/X747/X750/X7507/X7510/X7511/ |
| | | X7513/X7514/X7515/X7516/X753/X7537/X7538/X7539/X7540/X7541/X7542/ |
| | | X7543/X7544/X7545/X7546/X7547/X7549/X755/X7550/X7551/X7552/X7553/ |
| | | X7554/X7555/X7556/X7557/X7558/X756/X7560/X7561/X7562/X7563/X7564/ |
| | | X7566/X7568/X7569/X7570/X7571/X7572/X7573/X7574/X7575/X7576/ |
| | | X7577/X7578/X7579/X7580/X7581/X7582/X7583/X7584/X7585/X7586/X7587/ |
| | | X7588/X7589/X7662/X7664/X7665/X7911/X7912/X7913/X7914/X7915/X7916/ |
| | | X7918/X7920/X7922/X7925/X7926/X7928/X7930/X7931/X7936/X7942/ |
| | | X7945/X7946/X7948/X7955/X7957/X7961/X7963/X7965/X7967/X7972/X7974/ |
| | | X7978/X7992/X8542/X8543/X8545/X8546/X8547/X8548/X8552/X8553/X8631/ |
| | | X8632/X8640/X8641/X8642/X8643/X8644/X8645/X8646/X8647/X8648/ |
| | | X8649/X8651/X8652/X8653/X8654/X8655/X8656/X8657/X8658/X8659/X8660/ |
| | | X8661/X8662/X8663/X8664/X8665/X8666/X8667/X8668/X8669/X8670/X8671/ |
| | | X8672/X8673/X8674/X8675/X8975/X8976/X8977/X8979/X8980/X8985/ |
| | | X8987/X8989/X8990/X8995/X9000/X9003/X9008/X901/X9014/X9016/X9018/ |
| | | X9020/X9025/X9027/X903/X9031/X9036/X9038/X9041/X9043/X9046/X93/ |
| | | X94/X9566/X9567/X9569/X9571/X9619/X9620/X9621/X9622/X9623/X9624/ |
| | | X9625/X9627/X9628/X9629/X9630/X9631/X9632/X9633/X9634/X9635/X9636/ |
| | | X9637/X9897/X9898/X9903/X9909/X9912/X9917/X9926/X9932/X9937/X9939/ |
| | | X9943/X9948/X9950/X9967/X9969/X9972 |
| ASN_seq_SS_sspro8C | −1.28 | X104/X110/X1191/X1193/X1266/X1267/X1329/X1370/X1415/X1869/X193/X194/ |
| | | X195/X1952/X1953/X196/X2072/X2125/X23/X234/X235/X255/X2827/X2828/ |
| | | X3865/X39/X3947/X397/X399/X401/X402/X452/X453/X491/X492/X714/ |
| | | X715/X717/X719/X75/X76/X799/X830/X831/X862/X895 |
| ASN_seq_SS_sspro8E | −1.28 | X1164/X1166/X1167/X1168/X1169/X1170/X1172/X1174/X1175/X1177/X1181/ |
| | | X1182/X1183/X1185/X1186/X1206/X1207/X16/X17/X183/X1832/X1835/X1837/ |
| | | X1839/X184/X1840/X1843/X1847/X1848/X1849/X185/X1850/X1851/X1853/ |
| | | X1854/X1856/X1857/X1860/X1861/X1862/X1883/X190/X198/X25/X2704/ |
| | | X2705/X2708/X2713/X2723/X2724/X2725/X2727/X2728/X2729/X2732/X2733/ |
| | | X2735/X2738/X2739/X2740/X3750/X3762/X3767/X3773/X3774/X3776/ |
| | | X3777/X3779/X3780/X3781/X3782/X3783/X3784/X3787/X385/X386/X387/ |
| | | X389/X391/X395/X406/X407/X4921/X4922/X4928/X4933/X4950/X4951/X4952/ |
| | | X4955/X4956/X4957/X4958/X6159/X6161/X6162/X6163/X6185/X6194/X6195/ |
| | | X67/X68/X696/X698/X699/X702/X703/X704/X706/X708/X709/X71/X711/ |
| | | X727/X728/X729/X7379/X7387/X7388/X7406/X78/X8543 |
| ASN_seq_SS_sspro8S | −1.28 | X1164/X1166/X1167/X1168/X1169/X1170/X1172/X1175/X1176/X1177/X1179/ |
| | | X1180/X1181/X1182/X1183/X1184/X1185/X17/X1832/X1835/X1837/X1839/ |
| | | X184/X1840/X1843/X1847/X1848/X185/X1850/X1851/X1852/X1853/X1856/ |
| | | X1857/X1858/X1860/X1861/X189/X190/X2703/X2704/X2705/X2708/X2713/ |
| | | X2723/X2724/X2725/X2727/X2728/X2732/X2733/X2734/X2735/X2738/X2740/ |
| | | X3745/X3750/X3762/X3767/X3773/X3774/X3775/X3776/X3777/X3779/ |
| | | X3782/X3783/X385/X387/X388/X389/X391/X394/X395/X396/X4920/X4922/ |
| | | X4928/X4933/X4950/X4951/X4952/X4956/X6159/X6161/X6163/X6185/X6193/ |
| | | X6195/X68/X69/X696/X699/X70/X700/X701/X702/X703/X705/X706/X707/ |
| | | X709/X71/X710/X711/X7379/X7388/X7406/X8543/X8560 |
| ASN_seq_SS_sspro8T | −1.28 | X1179/X19/X3/X390/X705/X708 |
| ASN_seq_SS_ssproE | −1.28 | X1164/X1166/X1167/X1168/X1169/X1170/X1172/X1174/X1175/X1177/X1181/ |
| | | X1182/X1183/X1185/X1186/X1207/X16/X17/X183/X1832/X1835/X1837/X1839/ |
| | | X184/X1840/X1843/X1847/X1848/X1849/X185/X1850/X1851/X1853/X1854/ |
| | | X1856/X1857/X1860/X1861/X1862/X1883/X19/X190/X25/X2704/X2705/ |
| | | X2708/X2713/X2723/X2724/X2725/X2727/X2728/X2729/X2732/X2733/X2735/ |
| | | X2738/X2739/X2740/X3750/X3762/X3767/X3773/X3774/X3775/X3776/ |
| | | X3779/X3780/X3781/X3782/X3783/X3784/X3787/X385/X386/X387/X389/X391/ |
| | | X395/X4922/X4928/X4933/X4950/X4951/X4952/X4955/X4956/X4957/X4958/ |
| | | X6159/X6161/X6163/X6185/X6193/X6194/X6195/X67/X68/X696/X698/ |
| | | X699/X700/X702/X703/X704/X706/X708/X709/X71/X711/X729/X7379/X7388/ |
| | | X7406/X8543 |
| ASN_struct_aa_A | −1.28 | X1199/X1202/X1204/X1368/X1415/X1876/X1878/X197/X2125/X24/X2751/X403/ |
| | | X466/X722/X725/X77/X798/X83/X895 |
| ASN_struct_aa_C | −1.28 | X191 |
| ASN_struct_aa_D | −1.28 | X1174/X16/X183/X3775/X386/X67/X698/X704/X8560 |
| ASN_struct_aa_E | −1.28 | X1188/X1190/X1191/X1192/X1865/X1869/X1894/X2763/X3802/X398/X713/ |
| | | X716/X717 |
| ASN_struct_aa_F | −1.28 | X1/X1162/X1163/X1164/X1165/X1168/X1169/X1170/X1171/X1175/X1177/X1180/ |
| | | X1182/X1183/X1185/X1186/X1223/X1224/X1225/X1226/X1231/X1240/ |
| | | X1355/X17/X1831/X1833/X1834/X1836/X1839/X184/X1840/X1841/X1842/ |
| | | X1844/X185/X1850/X1851/X1853/X1854/X1856/X1860/X1861/X1862/X190/ |
| | | X1904/X1907/X1908/X1914/X1919/X2035/X2054/X216/X217/X26/X2699/X2700/ |
| | | X2701/X2702/X2704/X2706/X2707/X2709/X2712/X2715/X2716/X2717/ |
| | | X2718/X2727/X2728/X2729/X2732/X2733/X2735/X2738/X2739/X2777/X2782/ |
| | | X2799/X2923/X3742/X3743/X3744/X3746/X3747/X3749/X3752/X3753/X3754/ |
| | | X3755/X3760/X3761/X3763/X3766/X3767/X3768/X3769/X3779/X3780/ |
| | | X3781/X3782/X3783/X3787/X3829/X383/X385/X387/X389/X391/X395/X3970/ |
| | | X424/X426/X427/X4915/X4916/X4917/X4919/X4921/X4922/X4923/X4926/ |
| | | X4927/X4929/X4932/X4933/X4934/X4935/X4939/X4940/X4941/X4942/X4947/ |
| | | X4948/X4955/X4956/X4957/X4958/X4959/X6159/X6160/X6162/X6163/X6164/ |
| | | X6167/X6168/X6173/X6174/X6175/X6176/X6181/X6182/X6184/X6185/ |
| | | X6186/X6187/X6191/X6196/X6197/X68/X694/X696/X697/X699/X7/X701/X702/ |
| | | X703/X706/X709/X71/X711/X7379/X7381/X7387/X7388/X7389/X7394/ |
| | | X7395/X7396/X7397/X7401/X7403/X7404/X7407/X744/X748/X749/X751/X752/ |
| | | X853/X8543/X8545/X8555/X8556/X8558/X92 |
| ASN_struct_aa_G | −1.28 | X117/X13/X1354/X263/X45/X508/X509/X851/X852 |
| ASN_struct_aa_H | −1.28 | X1191/X1869/X717 |
| ASN_struct_aa_I | −1.28 | X104/X110/X1329/X234/X235/X255/X39/X452/X453/X491/X492/X799/X830/X831 |
| ASN_struct_aa_K | −1.28 | X107/X1174/X1219/X1223/X1224/X1225/X1226/X1228/X1231/X1232/X1233/ |
| | | X1237/X1240/X1267/X1372/X1898/X1899/X1901/X1904/X1905/X1907/X1908/ |
| | | X1910/X1914/X1916/X1919/X1920/X1923/X1953/X2035/X216/X217/X26/ |
| | | X267/X2770/X2771/X2773/X2777/X2779/X2782/X2783/X2785/X2793/X2796/ |
| | | X2799/X2828/X2922/X2923/X3816/X3819/X3825/X3829/X3833/X3834/X3865/ |
| | | X3966/X3970/X424/X426/X427/X428/X4982/X4984/X5120/X514/X515/ |
| | | X698/X7/X744/X746/X748/X749/X751/X752/X754/X864/X865/X92 |
| ASN_struct_aa_L | −1.28 | X49 |
| ASN_struct_aa_M | −1.28 | X1/X1164/X1166/X1167/X1168/X1169/X1170/X1172/X1174/X1175/X1176/X1177/ |
| | | X1180/X1181/X1182/X1183/X1184/X1185/X16/X17/X183/X1832/X1835/ |
| | | X1837/X1839/X184/X1840/X1843/X1847/X1848/X1849/X185/X1850/X1851/ |
| | | X1852/X1853/X1856/X1857/X1858/X1860/X1861/X189/X19/X190/X2703/ |
| | | X2704/X2705/X2708/X2713/X2723/X2724/X2725/X2727/X2728/X2732/X2733/ |
| | | X2734/X2735/X2738/X2739/X2740/X3/X3745/X3750/X3762/X3773/X3774/ |
| | | X3775/X3776/X3777/X3779/X3781/X3782/X3783/X3787/X385/X386/X387/ |
| | | X388/X389/X391/X395/X396/X4920/X4922/X4928/X4950/X4951/X4952/X4955/ |
| | | X4956/X4958/X4959/X6159/X6161/X6163/X6193/X6195/X6196/X67/X68/ |
| | | X696/X698/X699/X70/X700/X701/X702/X703/X704/X706/X707/X709/X71/ |
| | | X710/X711/X7379/X7388/X7406/X7407/X8543 |
| ASN_struct_aa_P | −1.28 | X1/X1164/X1169/X1172/X1176/X1179/X1181/X1184/X17/X1837/X184/X1840/ |
| | | X1843/X1852/X1857/X187/X189/X19/X191/X192/X21/X2708/X2713/X2734/ |
| | | X3/X3762/X385/X388/X390/X392/X394/X396/X68/X696/X70/X700/X703/ |
| | | X705/X707/X708/X710/X73 |
| ASN_struct_aa_Q | −1.28 | X1/X1164/X1165/X1167/X1169/X1170/X1172/X1177/X1180/X1181/X1183/X1185/ |
| | | X1186/X16/X17/X183/X1832/X1833/X1835/X1837/X184/X1840/X1841/ |
| | | X1843/X1848/X1849/X185/X1851/X1853/X1854/X1856/X1858/X190/X2701/ |
| | | X2703/X2704/X2705/X2706/X2708/X2713/X2715/X2717/X2723/X2724/X2725/ |
| | | X2733/X2735/X2736/X3745/X3747/X3750/X3752/X3754/X3760/X3762/X3766/ |
| | | X3767/X3774/X3776/X3777/X3784/X385/X386/X389/X391/X395/X396/ |
| | | X4917/X4920/X4921/X4922/X4928/X4932/X4933/X4939/X4941/X4951/X4952/ |
| | | X6/X6159/X6161/X6162/X6163/X6168/X6184/X6185/X6194/X67/X68/X694/ |
| | | X696/X699/X701/X703/X704/X706/X707/X71/X711/X7379/X7387/X7388/X8543 |
| ASN_struct_aa_R | −1.28 | X1162/X1163/X1164/X1165/X1166/X1167/X1168/X1169/X1170/X1171/X1172/ |
| | | X1175/X1176/X1177/X1179/X1180/X1181/X1182/X1183/X1184/X1185/X1186/ |
| | | X1223/X1224/X1225/X1226/X1231/X1240/X1831/X1833/X1834/X1835/ |
| | | X1836/X1837/X1839/X184/X1840/X1841/X1842/X1843/X1844/X1847/X1848/ |
| | | X1849/X185/X1850/X1851/X1852/X1853/X1854/X1856/X1857/X1858/X1860/ |
| | | X1861/X1862/X187/X189/X19/X190/X1904/X1907/X1908/X1914/X1919/X192/ |
| | | X2035/X21/X216/X217/X26/X2699/X2700/X2701/X2702/X2704/X2706/ |
| | | X2707/X2708/X2709/X2712/X2713/X2715/X2716/X2717/X2718/X2723/X2724/ |
| | | X2725/X2727/X2728/X2729/X2732/X2733/X2734/X2735/X2736/X2738/X2739/ |
| | | X2740/X2777/X2782/X2799/X2922/X2923/X3/X3742/X3744/X3746/X3747/ |
| | | X3749/X3752/X3753/X3754/X3755/X3760/X3761/X3762/X3763/X3766/X3767/ |
| | | X3773/X3774/X3775/X3776/X3777/X3779/X3780/X3781/X3782/X3783/ |
| | | X3784/X3787/X3829/X383/X385/X387/X388/X389/X390/X391/X392/X394/ |
| | | X395/X396/X3966/X3970/X424/X426/X427/X4916/X4917/X4919/X4920/X4921/ |
| | | X4923/X4927/X4929/X4932/X4933/X4939/X4940/X4941/X4942/X4950/X4951/ |
| | | X4952/X4955/X4956/X4957/X4958/X4959/X5120/X6159/X6162/X6164/ |
| | | X6167/X6168/X6184/X6185/X6186/X6193/X6194/X6195/X6196/X6197/X694/ |
| | | X696/X697/X699/X7/X70/X700/X701/X702/X703/X705/X706/X707/X708/ |
| | | X709/X71/X710/X711/X73/X7379/X7387/X7389/X7406/X7407/X744/X748/X749/ |
| | | X751/X752/X8543/X92 |
| ASN_struct_aa_S | −1.28 | X1/X1164/X1169/X1172/X1223/X1224/X1225/X1226/X1231/X1240/X1355/X1464/ |
| | | X17/X1832/X1837/X184/X1840/X1843/X188/X1904/X1907/X1908/X1919/ |
| | | X2054/X216/X217/X2186/X26/X2705/X2708/X2713/X2782/X2799/X3116/ |
| | | X3117/X3750/X3762/X385/X393/X424/X426/X427/X68/X696/X7/X703/X744/ |
| | | X748/X749/X751/X752/X853/X92 |
| ASN_struct_aa_T | −1.28 | X1206/X198/X25/X406/X407/X727/X728/X78 |
| ASN_struct_aa_V | −1.28 | X1162/X1163/X1164/X1165/X1166/X1167/X1169/X1170/X1171/X1172/X1174/ |
| | | X1175/X1177/X1180/X1181/X1183/X1185/X1186/X16/X17/X183/X1831/X1832/ |
| | | X1833/X1834/X1835/X1836/X1837/X184/X1840/X1841/X1842/X1844/ |
| | | X1847/X1848/X1849/X185/X1851/X1853/X1854/X1856/X1858/X188/X190/X2699/ |
| | | X2700/X2701/X2702/X2705/X2706/X2707/X2709/X2712/X2713/X2715/ |
| | | X2716/X2717/X2718/X2723/X2724/X2725/X2733/X2735/X2736/X3742/X3743/ |
| | | X3744/X3746/X3747/X3749/X3750/X3752/X3753/X3754/X3755/X3760/X3761/ |
| | | X3763/X3766/X3768/X3769/X3774/X3776/X383/X385/X386/X389/X391/ |
| | | X395/X4915/X4916/X4917/X4919/X4921/X4922/X4926/X4927/X4928/X4929/ |
| | | X4932/X4934/X4935/X4939/X4940/X4941/X4942/X4947/X4948/X6/X6159/ |
| | | X6160/X6161/X6162/X6163/X6167/X6168/X6170/X6173/X6174/X6175/X6176/ |
| | | X6181/X6182/X6184/X6186/X6187/X67/X68/X694/X696/X697/X698/X699/ |
| | | X701/X703/X704/X706/X709/X71/X711/X7379/X7381/X7382/X7384/X7387/ |
| | | X7388/X7394/X7396/X7403/X8543/X8545/X8546/X8547/X8552/X9567/X9570 |
| ASN_struct_SS_dsspE | −1.28 | X1163/X1167/X1168/X1170/X1171/X1175/X1177/X1180/X1182/X1183/X1185/ |
| | | X1206/X1834/X1836/X1839/X1842/X185/X1850/X1851/X1853/X1856/X1858/ |
| | | X1860/X1861/X190/X198/X25/X2707/X2712/X2727/X2728/X2733/X2735/ |
| | | X2738/X3761/X3779/X3783/X383/X387/X389/X391/X395/X406/X407/X4956/ |
| | | X6/X697/X699/X701/X702/X706/X709/X71/X711/X727/X728/X78 |
| ASN_struct_SS_dsspH | −1.28 | X1206/X1207/X1883/X198/X25/X406/X407/X727/X728/X729/X78 |
| ASN_struct_SS_dsspS | −1.28 | X1162/X1163/X1164/X1165/X1166/X1168/X1169/X1170/X1171/X1172/X1175/ |
| | | X1176/X1177/X1179/X1182/X1183/X1184/X1185/X1186/X1219/X1223/X1224/ |
| | | X1225/X1226/X1228/X1231/X1232/X1233/X1237/X1240/X1831/X1832/ |
| | | X1833/X1834/X1835/X1836/X1837/X1839/X184/X1840/X1841/X1842/X1843/ |
| | | X1844/X1847/X185/X1850/X1851/X1852/X1853/X1854/X1856/X1857/X1858/ |
| | | X1861/X1862/X189/X1898/X1899/X190/X1901/X1904/X1905/X1907/X1908/ |
| | | X1910/X1914/X1916/X1919/X1920/X1923/X2035/X216/X217/X26/X2699/ |
| | | X2700/X2701/X2702/X2703/X2704/X2705/X2706/X2707/X2708/X2709/X2712/ |
| | | X2713/X2715/X2716/X2717/X2718/X2723/X2724/X2728/X2729/X2732/X2733/ |
| | | X2734/X2735/X2736/X2738/X2739/X2740/X2770/X2771/X2773/X2777/ |
| | | X2779/X2782/X2783/X2785/X2793/X2796/X2799/X2922/X2923/X3742/X3743/ |
| | | X3744/X3745/X3746/X3747/X3749/X3750/X3752/X3753/X3754/X3755/X3760/ |
| | | X3761/X3762/X3763/X3766/X3767/X3768/X3769/X3774/X3775/X3776/ |
| | | X3777/X3779/X3780/X3781/X3782/X3783/X3784/X3787/X3816/X3819/X3825/ |
| | | X3829/X383/X3833/X3834/X385/X387/X388/X389/X391/X395/X396/X3966/ |
| | | X3970/X424/X426/X427/X428/X4916/X4917/X4919/X4920/X4922/X4923/ |
| | | X4926/X4927/X4929/X4932/X4933/X4934/X4935/X4939/X4940/X4941/X4942/ |
| | | X4947/X4948/X4951/X4952/X4955/X4957/X4958/X4959/X4982/X4984/X5120/ |
| | | X6163/X6164/X6167/X6168/X6173/X6174/X6175/X6176/X6181/X6182/ |
| | | X6184/X6185/X6186/X6187/X6191/X6194/X6195/X6196/X6197/X694/X696/ |
| | | X697/X699/X7/X70/X700/X701/X702/X703/X705/X706/X707/X709/X71/X710/ |
| | | X711/X7388/X7389/X7395/X7396/X7397/X7401/X7403/X7404/X7406/X7407/ |
| | | X744/X746/X748/X749/X751/X752/X754/X8555/X8556/X8558/X8560/X92 |
| ASN_struct_SS_dsspT | −1.28 | X1/X1164/X1169/X1172/X1176/X1179/X1184/X17/X1837/X184/X1840/X1843/ |
| | | X1844/X1852/X1857/X189/X19/X2703/X2708/X2709/X2713/X2717/X2734/ |
| | | X3/X3745/X3754/X3762/X3763/X3767/X385/X388/X394/X396/X4920/X4933/ |
| | | X4941/X6185/X68/X696/X70/X700/X703/X705/X707/X710 |
| SER.THR_seq_aaAll_E | −1.28 | X119/X149/X211/X27/X32/X380/X456/X6/X781/X90/X93 |
| SER.THR_seq_aaAll_H | −1.28 | X19/X2/X204/X237/X28/X495/X68/X81/X87 |
| SER.THR_seq_aaAll_P | −1.28 | X1/X116/X96 |
| SER.THR_seq_aaAll_Q | −1.28 | X29 |
| SER.THR_seq_aaAll_S | −1.28 | X29 |
| SER.THR_seq_aaDown_P | −1.28 | X1 |
| SER.THR_seq_aaUp_P | −1.28 | X1 |
| SER.THR_struct_aa_P | −1.28 | X1/X116 |
| SER.THR_struct_SS_dsspH | −1.28 | X2/X28/X87 |
| ASN_seq_aaAll_A | −2.33 | X1344/X1345/X1346/X1347/X1529/X2042/X2043/X2044/X2045/X2285/X258/ |
| | | X2932/X2933/X2934/X3980/X3981/X502/X843/X844 |
| ASN_seq_aaAll_C | −2.33 | X1316/X1317/X1318/X1320/X1321/X1322/X2011/X2012/X2013/X2015/X2017/ |
| | | X2021/X2022/X2023/X253/X2895/X2899/X2901/X2904/X2910/X2913/X2914/ |
| | | X3946/X3953/X3955/X3958/X3959/X485/X487/X5106/X5108/X822/X823/X824 |
| ASN_seq_aaAll_D | −2.33 | X117/X13/X1354/X191/X263/X45/X508/X509/X851/X852 |
| ASN_seq_aaAll_E | −2.33 | X10459/X1219/X1220/X1221/X1222/X1223/X1224/X1225/X1226/X1227/X1228/ |
| | | X1229/X1230/X1231/X1232/X1233/X1234/X1235/X1236/X1237/X1238/X1239/ |
| | | X1240/X1431/X1432/X1433/X1435/X1437/X1557/X1895/X1896/X1897/ |
| | | X1898/X1899/X1900/X1901/X1902/X1903/X1904/X1905/X1906/X1907/X1908/ |
| | | X1909/X1910/X1911/X1912/X1913/X1914/X1915/X1916/X1917/X1918/X1919/ |
| | | X1920/X1921/X1922/X1923/X1924/X1925/X2035/X2036/X2145/X2146/ |
| | | X2148/X2149/X215/X2150/X2151/X2152/X2154/X2157/X216/X2160/X217/ |
| | | X218/X2331/X2332/X26/X2764/X2765/X2766/X2767/X2768/X2769/X2770/X2771/ |
| | | X2772/X2773/X2774/X2775/X2776/X2777/X2778/X2779/X2780/X2781/ |
| | | X2782/X2783/X2784/X2785/X2786/X2787/X2788/X2789/X2790/X2791/X2792/ |
| | | X2793/X2794/X2795/X2796/X2797/X2798/X2799/X2922/X2923/X2924/X2925/ |
| | | X2926/X2927/X2928/X3032/X3033/X3034/X3035/X3036/X3038/X3039/ |
| | | X3040/X3042/X3044/X3045/X3046/X3050/X3051/X3052/X3053/X3056/X3059/ |
| | | X3060/X3061/X3062/X32/X3285/X3286/X3287/X3293/X3803/X3804/X3805/ |
| | | X3806/X3807/X3808/X3810/X3811/X3812/X3813/X3814/X3815/X3816/X3817/ |
| | | X3818/X3819/X3820/X3821/X3822/X3823/X3824/X3825/X3826/X3827/ |
| | | X3828/X3829/X3830/X3831/X3832/X3833/X3834/X3835/X3836/X3966/X3967/ |
| | | X3968/X3969/X3970/X3972/X3973/X3974/X3975/X3976/X3977/X3978/X4066/ |
| | | X4067/X4069/X4070/X4071/X4072/X4073/X4074/X4075/X4076/X4078/ |
| | | X4079/X4080/X4081/X4082/X4083/X4087/X4088/X4089/X4090/X4093/X4095/ |
| | | X4096/X4097/X4098/X4099/X4102/X4103/X4104/X4105/X4108/X4109/X4111/ |
| | | X4112/X4113/X424/X425/X426/X427/X428/X429/X430/X4373/X4374/ |
| | | X4376/X4378/X4384/X4389/X4392/X4394/X4969/X4970/X4971/X4973/X4974/ |
| | | X4975/X4976/X4977/X4978/X4979/X4980/X4981/X4982/X4983/X4984/X4985/ |
| | | X4986/X4987/X4988/X4989/X4990/X4991/X5119/X5120/X5123/X5124/ |
| | | X5125/X5127/X5128/X5129/X5130/X5131/X5132/X5133/X5134/X5135/X5200/ |
| | | X5201/X5202/X5203/X5204/X5205/X5206/X5207/X5208/X5210/X5211/X5212/ |
| | | X5213/X5214/X5215/X5217/X5218/X5219/X5220/X5221/X5222/X5225/ |
| | | X5227/X5228/X5229/X5230/X5231/X5232/X5233/X5237/X5238/X5239/X5240/ |
| | | X5241/X5242/X5243/X5244/X5245/X5248/X5249/X5252/X5253/X5254/X5255/ |
| | | X5256/X5257/X5258/X5259/X5260/X5309/X5310/X532/X5564/X5565/X5566/ |
| | | X5570/X5575/X5580/X5583/X5586/X5588/X5590/X5594/X6200/X6201/ |
| | | X6202/X6204/X6205/X6206/X6207/X6208/X6210/X6211/X6325/X6328/X6329/ |
| | | X6330/X6331/X6332/X6334/X6335/X6336/X6337/X6338/X6339/X6381/X6382/ |
| | | X6383/X6384/X6385/X6386/X6387/X6388/X6389/X6391/X6392/X6393/ |
| | | X6394/X6395/X6396/X6397/X6399/X6400/X6401/X6403/X6404/X6405/X6406/ |
| | | X6407/X6408/X6409/X6413/X6414/X6415/X6416/X6417/X6418/X6419/X6420/ |
| | | X6422/X6424/X6425/X6426/X6427/X6428/X6430/X6431/X6432/X6433/ |
| | | X6434/X6435/X6436/X6437/X6438/X6439/X6440/X6441/X6442/X6443/X6503/ |
| | | X6506/X6507/X6760/X6762/X6764/X6767/X6772/X6773/X6777/X6787/X6793/ |
| | | X6796/X6798/X6812/X7/X7409/X7412/X744/X745/X746/X747/X748/X749/ |
| | | X750/X7507/X751/X7510/X7511/X7513/X7514/X7515/X7516/X752/X753/ |
| | | X7537/X7538/X7539/X754/X7540/X7541/X7544/X7545/X7547/X7549/X755/ |
| | | X7550/X7551/X7553/X7554/X7555/X7556/X7557/X7558/X756/X7560/X7561/ |
| | | X7562/X7563/X7564/X7566/X7568/X7570/X7571/X7572/X7573/X7574/X7575/ |
| | | X7576/X7577/X7578/X7579/X7580/X7581/X7582/X7583/X7584/X7585/ |
| | | X7586/X7587/X7589/X7662/X7664/X7665/X7912/X7914/X7916/X7922/X7926/ |
| | | X7931/X7946/X7948/X7961/X8631/X8632/X8642/X8645/X8646/X8647/X8648/ |
| | | X8651/X8654/X8655/X8657/X8658/X8659/X8660/X8661/X8664/X8665/ |
| | | X8666/X8667/X8668/X8669/X8670/X8673/X8674/X8977/X8979/X8990/X9000/ |
| | | X901/X903/X9031/X9041/X9043/X92/X93/X94/X9624/X9625/X9630/X9631/ |
| | | X9632/X9634/X9635/X9903/X9932 |
| ASN_seq_aaAll_F | −2.33 | X10428/X10494/X1356/X1358/X1359/X1361/X1365/X1440/X1502/X18/X2055/ |
| | | X2056/X2057/X2062/X2063/X2066/X2067/X2069/X2120/X2162/X2166/X2244/ |
| | | X254/X2718/X2944/X2946/X2950/X2951/X2952/X2953/X2955/X2956/X2957/ |
| | | X2958/X2960/X2962/X2996/X2997/X3002/X3004/X3065/X3070/X3072/ |
| | | X3073/X3149/X3150/X3755/X3768/X3769/X3992/X3993/X3994/X3995/X3996/ |
| | | X3997/X3998/X4000/X4001/X4002/X4005/X4006/X4008/X4009/X4025/X4026/ |
| | | X4032/X4033/X4037/X4038/X4039/X4040/X4042/X4114/X4119/X4123/ |
| | | X4124/X4127/X4130/X4189/X4191/X4193/X489/X4923/X4934/X4935/X4942/ |
| | | X4947/X4948/X511/X5141/X5142/X5143/X5144/X5145/X5147/X5150/X5151/ |
| | | X5152/X5153/X5154/X5155/X5161/X5164/X5165/X5169/X5170/X5171/X5172/ |
| | | X5173/X5174/X5178/X5179/X5180/X5181/X5264/X5273/X5276/X5282/ |
| | | X5283/X5293/X5335/X5340/X5341/X5344/X6164/X6170/X6176/X6181/X6182/ |
| | | X6186/X6187/X6191/X6344/X6346/X6347/X6348/X6349/X6350/X6351/X6352/ |
| | | X6353/X6355/X6356/X6357/X6358/X6359/X6363/X6364/X6365/X6366/ |
| | | X6367/X6368/X6369/X6452/X6461/X6462/X6463/X6476/X6490/X6491/X6525/ |
| | | X6528/X6531/X7384/X7389/X7392/X7393/X7396/X7397/X7401/X7403/X7404/ |
| | | X7517/X7518/X7519/X7520/X7521/X7522/X7524/X7525/X7526/X7527/ |
| | | X7528/X7529/X7530/X7593/X7615/X7617/X7618/X7621/X7651/X7652/X7683/ |
| | | X7689/X854/X8547/X855/X8550/X8551/X8552/X8555/X8556/X8558/X858/ |
| | | X8633/X8634/X8635/X8636/X8707/X8709/X8712/X8715/X8716/X8742/X8772/ |
| | | X907/X9570/X9573/X9574/X9575/X9672/X9674/X9677/X9689 |
| ASN_seq_aaAll_G | −2.33 | X170 |
| ASN_seq_aaAll_H | −2.33 | X1358/X1361/X1362/X1363/X1364/X1365/X2056/X2060/X2062/X2064/X2065/ |
| | | X2066/X2067/X2068/X2069/X265/X29/X2948/X2950/X2952/X2955/X2956/ |
| | | X2958/X2959/X2960/X2961/X2962/X3072/X3994/X3997/X4000/X4001/X4003/ |
| | | X4005/X4007/X4008/X4009/X4123/X511/X512/X5143/X5148/X5150/X5152/ |
| | | X5153/X5155/X5282/X6347/X6350/X6351/X6461/X7518/X854/X857/X858/ |
| | | X859/X90 |
| ASN_seq_aaAll_K | −2.33 | X1266/X1952/X2827 |
| ASN_seq_aaAll_M | −2.33 | X1358/X1361/X1365/X2056/X2062/X2066/X2067/X2069/X2950/X2952/X2955/ |
| | | X2956/X2958/X2960/X2962/X3994/X3997/X4000/X4001/X4005/X4008/X4009/ |
| | | X511/X5143/X5150/X5152/X5153/X5155/X6347/X6350/X6351/X7518/X854/ |
| | | X858 |
| ASN_seq_aaAll_N | −2.33 | X183/X386/X6/X67/X698 |
| ASN_seq_aaAll_P | −2.33 | X1188/X1189/X1190/X1191/X1192/X1193/X1199/X1202/X1204/X1865/X1868/ |
| | | X1869/X1876/X1878/X1880/X1894/X193/X194/X195/X196/X197/X23/X24/ |
| | | X2741/X2751/X2753/X2763/X3794/X3802/X397/X398/X399/X400/X401/X402/ |
| | | X403/X713/X714/X715/X716/X717/X718/X719/X722/X725/X75/X76/X77 |
| ASN_seq_aaAll_R | −2.33 | X10494/X117/X13/X1354/X1355/X1440/X1464/X1479/X191/X2054/X2120/X2162/ |
| | | X2166/X2186/X2211/X2212/X254/X263/X2996/X2997/X3002/X3004/X3065/ |
| | | X3070/X3072/X3073/X3115/X3116/X3117/X4025/X4026/X4032/X4033/ |
| | | X4037/X4038/X4039/X4040/X4042/X4114/X4119/X4123/X4124/X4127/X4166/ |
| | | X45/X489/X508/X509/X5161/X5164/X5165/X5169/X5170/X5171/X5172/ |
| | | X5173/X5174/X5178/X5179/X5180/X5181/X5264/X5273/X5282/X5283/X5293/ |
| | | X6353/X6355/X6356/X6357/X6358/X6359/X6363/X6364/X6365/X6366/X6367/ |
| | | X6368/X6369/X6452/X6461/X6462/X6463/X6490/X6491/X74/X7521/X7522/ |
| | | X7524/X7525/X7526/X7527/X7528/X7529/X7530/X7593/X7615/X7618/ |
| | | X7621/X7651/X7652/X851/X852/X853/X8633/X8634/X8635/X8636/X8707/X8709/ |
| | | X8712/X8715/X8742/X907/X9672/X9674/X9677/X9689 |
| ASN_seq_aaAll_S | −2.33 | X188/X191/X393 |
| ASN_seq_aaAll_T | −2.33 | X1294/X1295/X1296/X1316/X1317/X1318/X1320/X1321/X1322/X170/X1991/ |
| | | X1995/X1996/X2011/X2012/X2013/X2015/X2016/X2017/X2018/X2021/X2022/ |
| | | X2023/X245/X253/X27/X2882/X2887/X2895/X2896/X2899/X2900/X2901/ |
| | | X2902/X2904/X2910/X2913/X2914/X3936/X3940/X3946/X3953/X3954/X3955/ |
| | | X3956/X3958/X3959/X470/X485/X487/X5103/X5106/X5107/X5108/X5109/ |
| | | X6314/X80/X801/X802/X822/X823/X824 |
| ASN_seq_aaAll_V | −2.33 | X1267/X1953/X2828/X3865 |
| ASN_seq_aaDown_A | −2.33 | X1358/X1361/X1362/X1363/X1364/X1365/X2056/X2060/X2062/X2064/X2065/ |
| | | X2066/X2067/X2068/X2069/X265/X29/X2948/X2950/X2952/X2955/X2956/ |
| | | X2958/X2959/X2960/X2961/X2962/X3072/X3994/X3997/X4000/X4001/X4003/ |
| | | X4005/X4007/X4008/X4009/X4123/X4132/X511/X512/X5143/X5148/X5150/ |
| | | X5152/X5153/X5155/X5278/X5282/X5289/X6347/X6350/X6351/X6461/X6470/ |
| | | X6478/X7518/X7620/X7639/X854/X857/X858/X859/X8714/X90 |
| ASN_seq_aaDown_C | −2.33 | X1267/X1953/X2828/X3865 |
| ASN_seq_aaDown_D | −2.33 | X1415/X2125/X895 |
| ASN_seq_aaDown_E | −2.33 | X107/X1367/X1371/X1372/X2073/X249/X267/X3072/X4123/X4132/X513/X514/ |
| | | X515/X5278/X5282/X5289/X6461/X6470/X6478/X7620/X7639/X83/X8560/ |
| | | X860/X863/X864/X865/X8714/X9575 |
| ASN_seq_aaDown_F | −2.33 | X1/X1162/X1163/X1165/X1171/X1186/X1199/X1202/X1204/X1357/X1360/X1362/ |
| | | X1363/X1364/X1831/X1832/X1833/X1834/X1836/X1841/X1842/X1844/ |
| | | X1876/X1878/X197/X2058/X2059/X2060/X2061/X2065/X2068/X214/X265/ |
| | | X2699/X2700/X2701/X2702/X2705/X2706/X2707/X2709/X2712/X2715/X2716/ |
| | | X2717/X2751/X29/X2945/X2948/X2949/X2954/X2961/X3742/X3743/X3744/ |
| | | X3746/X3749/X3750/X3752/X3753/X3754/X3760/X3761/X3763/X383/X3991/ |
| | | X4003/X4004/X403/X4915/X4916/X4919/X4926/X4927/X4929/X4939/X4940/ |
| | | X4941/X510/X512/X5146/X6160/X6167/X6173/X6174/X6175/X694/X697/ |
| | | X722/X725/X7381/X7382/X7394/X7395/X84/X8545/X8546/X856/X857/X859/ |
| | | X90/X9567 |
| ASN_seq_aaDown_G | −2.33 | X1188/X1190/X1192/X1865/X1868/X191/X2741/X398/X713/X716 |
| ASN_seq_aaDown_I | −2.33 | X1267/X1953/X2828/X3865 |
| ASN_seq_aaDown_K | −2.33 | X3768/X3769/X4934/X4935/X4947/X4948/X6176/X6181/X6182/X6186/X6187/ |
| | | X69/X7396/X7403/X7404 |
| ASN_seq_aaDown_L | −2.33 | X10494/X2120/X2947/X2996/X2997/X3002/X3004/X3072/X3999/X4025/X4026/ |
| | | X4032/X4033/X4037/X4038/X4039/X4040/X4042/X4123/X4132/X5149/X5161/ |
| | | X5164/X5165/X5169/X5170/X5171/X5172/X5173/X5174/X5178/X5179/ |
| | | X5180/X5181/X5278/X5282/X5289/X5293/X6345/X6353/X6355/X6356/X6357/ |
| | | X6358/X6359/X6363/X6364/X6365/X6366/X6367/X6368/X6369/X6461/X6462/ |
| | | X6470/X6478/X6490/X6491/X7521/X7522/X7524/X7525/X7526/X7527/ |
| | | X7528/X7529/X7530/X7615/X7618/X7620/X7621/X7639/X7651/X7652/X8633/ |
| | | X8634/X8635/X8636/X8707/X8709/X8712/X8714/X8715/X8742/X9672/X9674/ |
| | | X9677/X9689 |
| ASN_seq_aaDown_M | −2.33 | X1163/X1171/X1834/X1836/X1842/X2700/X2707/X2712/X2739/X3743/X3749/ |
| | | X3761/X3780/X383/X4915/X4926/X4927/X4929/X4957/X6160/X6173/X6174/ |
| | | X6175/X697/X7381/X7394 |
| ASN_seq_aaDown_P | −2.33 | X1368/X1880/X1881/X24/X2753/X2754/X3794/X3795/X466/X77/X798 |
| ASN_seq_aaDown_Q | −2.33 | X104/X107/X110/X1329/X1330/X1368/X1372/X234/X235/X255/X267/X39/X452/ |
| | | X453/X466/X491/X492/X493/X514/X515/X798/X799/X830/X831/X832/ |
| | | X864/X865 |
| ASN_seq_aaDown_R | −2.33 | X1834/X1844/X2709/X2712/X2718/X3755/X3761/X3763/X3768/X4923/X4934/ |
| | | X4942/X6164/X6176/X6186/X6187/X7389 |
| ASN_seq_aaDown_T | −2.33 | X115/X12/X1266/X1267/X1294/X1295/X1296/X1316/X1317/X1318/X1320/X1321/ |
| | | X1322/X1350/X1351/X1352/X1950/X1951/X1952/X1953/X1995/X1996/ |
| | | X2011/X2012/X2013/X2015/X2016/X2021/X2022/X2023/X2050/X2051/X2287/ |
| | | X245/X253/X260/X27/X2825/X2826/X2827/X2828/X2887/X2895/X2896/ |
| | | X2899/X2900/X2904/X2910/X2913/X2914/X2941/X3226/X3863/X3864/X3865/ |
| | | X3940/X3946/X3953/X3954/X3958/X3959/X4295/X44/X470/X485/X487/X504/ |
| | | X5103/X5106/X5107/X5318/X5385/X6314/X6895/X80/X801/X802/X822/ |
| | | X823/X824/X846/X847 |
| ASN_seq_aaDown_V | −2.33 | X1316/X1317/X1318/X1320/X1321/X1322/X18/X2011/X2012/X2013/X2015/ |
| | | X2017/X2021/X2022/X2023/X253/X2895/X2899/X2901/X2904/X2910/X2913/ |
| | | X2914/X3946/X3953/X3955/X3958/X3959/X485/X487/X5106/X5108/X822/ |
| | | X823/X824 |
| ASN_seq_aaDown_Y | −2.33 | X191/X2718/X3755/X4942/X74 |
| ASN_seq_aaUp_A | −2.33 | X1188/X1190/X1191/X1192/X1193/X1266/X1267/X1274/X1277/X1281/X1287/ |
| | | X1289/X1865/X1866/X1868/X1869/X1894/X193/X194/X195/X1952/X1953/ |
| | | X196/X1971/X1974/X1976/X1978/X1984/X23/X239/X2741/X2742/X2763/X2827/ |
| | | X2828/X2857/X2861/X2868/X2870/X3802/X3865/X3922/X3935/X3947/ |
| | | X397/X398/X399/X401/X402/X462/X5098/X713/X714/X715/X716/X717/X719/ |
| | | X75/X76/X787/X793 |
| ASN_seq_aaUp_D | −2.33 | X1357/X1358/X1360/X1361/X1362/X1363/X1364/X1365/X191/X2056/X2058/ |
| | | X2059/X2060/X2061/X2062/X2065/X2066/X2067/X2068/X2069/X214/X265/ |
| | | X29/X2945/X2948/X2949/X2950/X2952/X2954/X2955/X2956/X2958/X2960/ |
| | | X2961/X2962/X3991/X3994/X3997/X4000/X4001/X4003/X4004/X4005/X4008/ |
| | | X4009/X510/X511/X512/X5143/X5146/X5150/X5152/X5153/X5155/X6347/ |
| | | X6350/X6351/X74/X7518/X84/X854/X856/X857/X858/X859/X90 |
| ASN_seq_aaUp_F | −2.33 | X4132/X5278/X5289/X6470/X6478/X69/X7620/X7639/X8714 |
| ASN_seq_aaUp_G | −2.33 | X1358/X1361/X1365/X1440/X2056/X2062/X2066/X2067/X2069/X2162/X2166/ |
| | | X254/X2947/X2950/X2952/X2955/X2956/X2958/X2960/X2962/X3065/X3070/ |
| | | X3073/X3994/X3997/X3999/X4000/X4001/X4005/X4008/X4009/X4114/X4119/ |
| | | X4124/X4127/X4130/X4132/X489/X511/X5143/X5149/X5150/X5152/X5153/ |
| | | X5155/X5264/X5273/X5276/X5278/X5283/X5289/X6345/X6347/X6350/ |
| | | X6351/X6452/X6463/X6470/X6476/X6478/X7518/X7593/X7617/X7620/X7639/ |
| | | X854/X858/X8714/X907 |
| ASN_seq_aaUp_H | −2.33 | X1358/X1361/X1362/X1363/X1364/X1365/X2056/X2060/X2062/X2064/X2065/ |
| | | X2066/X2067/X2068/X2069/X265/X29/X2948/X2950/X2952/X2955/X2956/ |
| | | X2958/X2959/X2960/X2961/X2962/X3994/X3997/X4000/X4001/X4003/X4005/ |
| | | X4007/X4008/X4009/X511/X512/X5143/X5148/X5150/X5152/X5153/X5155/ |
| | | X6347/X6350/X6351/X7518/X854/X857/X858/X859/X90 |
| ASN_seq_aaUp_I | −2.33 | X1362/X1363/X1364/X2060/X2064/X2065/X2068/X265/X29/X2948/X2959/X2961/ |
| | | X4003/X4007/X512/X5148/X69/X857/X859/X90 |
| ASN_seq_aaUp_K | −2.33 | X1266/X1267/X1952/X1953/X2827/X2828/X3865/X3947 |
| ASN_seq_aaUp_L | −2.33 | X170 |
| ASN_seq_aaUp_P | −2.33 | X1199/X1202/X1204/X1358/X1361/X1365/X1440/X1876/X1878/X1951/X197/ |
| | | X2056/X2062/X2066/X2067/X2069/X2162/X2166/X24/X254/X2751/X2826/ |
| | | X2947/X2950/X2952/X2955/X2956/X2958/X2960/X2962/X3065/X3070/X3073/ |
| | | X3864/X3994/X3997/X3999/X4000/X4001/X4005/X4008/X4009/X403/X4114/ |
| | | X4119/X4124/X4127/X4130/X489/X511/X5143/X5149/X5150/X5152/X5153/ |
| | | X5155/X5264/X5273/X5276/X5283/X6345/X6347/X6350/X6351/X6452/X6463/ |
| | | X6476/X722/X725/X7518/X7593/X7617/X77/X854/X858/X907 |
| ASN_seq_aaUp_R | −2.33 | X117/X13/X1354/X263/X45/X508/X509/X851/X852 |
| ASN_seq_aaUp_T | −2.33 | X1316/X1317/X1318/X1320/X1321/X1322/X1358/X1361/X1362/X1363/X1364/ |
| | | X1365/X2011/X2012/X2013/X2015/X2021/X2022/X2023/X2056/X2060/X2062/ |
| | | X2064/X2065/X2066/X2067/X2068/X2069/X253/X265/X2895/X2899/X29/ |
| | | X2904/X2910/X2913/X2914/X2948/X2950/X2952/X2955/X2956/X2958/X2959/ |
| | | X2960/X2961/X2962/X3072/X3946/X3953/X3958/X3959/X3994/X3997/ |
| | | X4000/X4001/X4003/X4005/X4007/X4008/X4009/X4123/X4132/X485/X487/ |
| | | X5106/X511/X512/X5143/X5148/X5150/X5152/X5153/X5155/X5278/X5282/ |
| | | X5289/X6347/X6350/X6351/X6461/X6470/X6478/X7518/X7620/X7639/X822/ |
| | | X823/X824/X854/X857/X858/X859/X8714/X90 |
| ASN_seq_aaUp_V | −2.33 | X1266/X1952/X2827 |
| ASN_seq_aaUp_W | −2.33 | X49 |
| ASN_seq_aaUp_Y | −2.33 | X74 |
| ASN_seq_RSA_accproe | −2.33 | X117/X1219/X1223/X1224/X1225/X1226/X1228/X1231/X1233/X1237/X1240/ |
| | | X13/X1354/X1355/X1479/X1898/X1899/X1901/X1904/X1907/X1908/X1910/ |
| | | X1914/X1916/X1919/X1920/X1923/X2035/X2054/X216/X217/X2212/X26/X263/ |
| | | X2770/X2771/X2773/X2777/X2779/X2782/X2785/X2793/X2796/X2799/ |
| | | X2922/X2923/X3116/X3816/X3819/X3825/X3829/X3833/X3834/X393/X3966/ |
| | | X3970/X4167/X424/X426/X427/X428/X45/X4982/X4984/X508/X509/X5120/ |
| | | X7/X744/X746/X748/X749/X751/X752/X754/X851/X852/X853/X92 |
| ASN_seq_SS_sspro8C | −2.33 | X1206/X1207/X1208/X1368/X1883/X1884/X1950/X198/X25/X2756/X2825/X3863/ |
| | | X406/X407/X466/X49/X5318/X727/X728/X729/X78/X798 |
| ASN_seq_SS_sspro8E | −2.33 | X10428/X1162/X1163/X1165/X1171/X1180/X1266/X1362/X1363/X1364/X18/ |
| | | X1831/X1833/X1834/X1836/X1841/X1842/X1844/X1858/X188/X1952/X2060/ |
| | | X2065/X2068/X265/X2699/X2700/X2701/X2702/X2706/X2707/X2709/X2712/ |
| | | X2715/X2716/X2717/X2736/X2827/X29/X2948/X2961/X3742/X3743/X3744/ |
| | | X3746/X3747/X3749/X3752/X3753/X3754/X3760/X3761/X3763/X3766/X3769/ |
| | | X383/X393/X4003/X4915/X4916/X4917/X4919/X4924/X4926/X4927/X4929/ |
| | | X4932/X4935/X4939/X4940/X4941/X4947/X4948/X512/X6/X6160/X6165/ |
| | | X6167/X6168/X6170/X6171/X6173/X6174/X6175/X6181/X6182/X6184/X |
| | | 6187/X6191/X69/X694/X697/X701/X7380/X7381/X7382/X7384/X7385/X7390/ |
| | | X7394/X7395/X7401/X7403/X7404/X8542/X8545/X8546/X8552/X8553/X8558/ |
| | | X857/X859/X90/X9567/X9569/X9575 |
| ASN_seq_SS_sspro8H | −2.33 | X6 |
| ASN_seq_SS_sspro8S | −2.33 | X10425/X10426/X11095/X1162/X1163/X1165/X1171/X1186/X1831/X1833/X1834/ |
| | | X1836/X1841/X1842/X1844/X1854/X1862/X2699/X2700/X2701/X2702/ |
| | | X2706/X2707/X2709/X2712/X2715/X2716/X2717/X2729/X2736/X2739/X3742/ |
| | | X3743/X3744/X3746/X3747/X3749/X3752/X3753/X3754/X3760/X3761/X3763/ |
| | | X3766/X3780/X3781/X3784/X3787/X383/X4915/X4916/X4917/X4919/ |
| | | X4921/X4924/X4926/X4927/X4929/X4932/X4939/X4940/X4941/X4947/X4948/ |
| | | X4955/X4957/X4958/X4959/X6160/X6162/X6165/X6167/X6168/X6170/X6171/ |
| | | X6173/X6174/X6175/X6181/X6182/X6184/X6191/X6194/X6196/X6197/ |
| | | X694/X697/X7380/X7381/X7382/X7384/X7385/X7387/X7390/X7392/X7394/ |
| | | X7395/X7397/X7401/X7403/X7404/X7407/X8542/X8545/X8546/X8547/X8548/ |
| | | X8550/X8552/X8553/X8555/X8556/X8558/X9566/X9567/X9569/X9570/X9571/ |
| | | X9573/X9575 |
| ASN_seq_SS_sspro8T | −2.33 | X105/X1274/X1283/X1284/X191/X1971/X1980/X241/X2857/X461/X464/X74/ |
| | | X789/X792/X795 |
| ASN_seq_SS_ssproE | −2.33 | X10426/X10428/X1162/X1163/X1165/X1171/X1180/X1266/X1362/X1363/X1364/ |
| | | X18/X1831/X1833/X1834/X1836/X1841/X1842/X1844/X1858/X188/X1952/ |
| | | X2060/X2065/X2068/X265/X2699/X2701/X2702/X2706/X2707/X2709/X2712/ |
| | | X2715/X2716/X2717/X2736/X2827/X29/X2948/X2961/X3742/X3743/X3744/ |
| | | X3746/X3747/X3752/X3753/X3754/X3760/X3761/X3763/X3766/X3769/ |
| | | X3777/X383/X393/X4003/X4915/X4916/X4917/X4919/X4921/X4924/X4926/ |
| | | X4929/X4932/X4935/X4939/X4940/X4941/X4947/X4948/X512/X6/X6160/X6162/ |
| | | X6165/X6167/X6168/X6170/X6171/X6173/X6174/X6175/X6181/X6182/ |
| | | X6184/X6187/X6191/X69/X694/X697/X701/X7380/X7381/X7382/X7384/X7385/ |
| | | X7387/X7390/X7394/X7395/X7401/X7403/X7404/X8542/X8545/X8546/X8548/ |
| | | X8552/X8553/X8558/X857/X859/X90/X9567/X9569/X9571/X9575 |
| ASN_seq_SS_ssproH | −2.33 | X6 |
| ASN_struct_aa_A | −2.33 | X105/X1206/X1207/X1208/X1266/X1267/X1283/X1284/X1317/X1320/X1321/ |
| | | X1322/X1426/X1883/X1884/X1951/X1952/X1953/X198/X1980/X2012/X2016/ |
| | | X2021/X2022/X2023/X2140/X241/X25/X253/X2756/X2826/X2827/X2828/ |
| | | X2896/X2900/X2910/X2913/X2914/X3864/X3865/X3940/X3947/X3954/X3958/ |
| | | X3959/X406/X407/X461/X464/X487/X49/X5103/X5107/X6314/X727/X728/ |
| | | X729/X78/X789/X792/X795/X823/X824/X899 |
| ASN_struct_aa_C | −2.33 | X74 |
| ASN_struct_aa_D | −2.33 | X1189/X1194/X1274/X1277/X1281/X1287/X1358/X1361/X1362/X1363/X1364/ |
| | | X1365/X1866/X1867/X1971/X1974/X1978/X2056/X2060/X2062/X2064/X2065/ |
| | | X2066/X2067/X2068/X2069/X239/X265/X2742/X2743/X2857/X2868/X29/ |
| | | X2948/X2950/X2952/X2955/X2956/X2958/X2959/X2960/X2961/X2962/X3935/ |
| | | X3994/X3997/X400/X4000/X4001/X4003/X4005/X4007/X4008/X4009/X462/ |
| | | X511/X512/X5143/X5148/X5150/X5152/X5153/X5155/X6347/X6350/X6351/ |
| | | X718/X7518/X787/X793/X83/X854/X857/X858/X859/X90 |
| ASN_struct_aa_E | −2.33 | X1189/X1194/X1867/X2743/X400/X718 |
| ASN_struct_aa_F | −2.33 | X117/X13/X1354/X18/X263/X4132/X45/X508/X509/X5278/X5289/X6470/X6478/ |
| | | X7620/X7639/X851/X852/X8714 |
| ASN_struct_aa_G | −2.33 | X191 |
| ASN_struct_aa_H | −2.33 | X105/X1189/X1193/X1194/X1274/X1283/X1284/X1358/X1361/X1362/X1363/ |
| | | X1364/X1365/X1867/X193/X194/X195/X196/X1971/X1980/X2056/X2060/X2062/ |
| | | X2064/X2065/X2066/X2067/X2068/X2069/X23/X241/X265/X2743/X2857/ |
| | | X29/X2948/X2950/X2952/X2955/X2956/X2958/X2959/X2960/X2961/X2962/ |
| | | X397/X399/X3994/X3997/X400/X4000/X4001/X4003/X4005/X4007/X4008/ |
| | | X4009/X401/X402/X461/X464/X511/X512/X5143/X5148/X5150/X5152/X5153/ |
| | | X5155/X6347/X6350/X6351/X714/X715/X718/X719/X75/X7518/X76/X789/ |
| | | X792/X795/X83/X854/X857/X858/X859/X90 |
| ASN_struct_aa_L | −2.33 | X170 |
| ASN_struct_aa_M | −2.33 | X10427/X10428/X11096/X11581/X1162/X1163/X1165/X1171/X1186/X1831/ |
| | | X1833/X1834/X1836/X1841/X1842/X1844/X1854/X1862/X2699/X2700/X2701/ |
| | | X2702/X2706/X2707/X2709/X2712/X2715/X2716/X2717/X2729/X2736/X3742/ |
| | | X3743/X3744/X3746/X3747/X3749/X3752/X3753/X3754/X3760/X3761/ |
| | | X3763/X3766/X3767/X3780/X3784/X383/X4915/X4916/X4917/X4919/X4921/ |
| | | X4926/X4927/X4929/X4932/X4933/X4939/X4940/X4941/X4957/X6160/X6162/ |
| | | X6167/X6168/X6173/X6174/X6175/X6184/X6185/X6194/X6197/X694/X697/ |
| | | X7381/X7382/X7387/X7393/X7394/X7395/X8544/X8545/X8546/X8551/X9567/ |
| | | X9568/X9574/X9575 |
| ASN_struct_aa_N | −2.33 | X183/X386/X6/X67/X698 |
| ASN_struct_aa_P | −2.33 | X1358/X1361/X1365/X2056/X2062/X2066/X2067/X2069/X2950/X2952/X2955/ |
| | | X2956/X2958/X2960/X2962/X3994/X3997/X4000/X4001/X4005/X4008/X4009/ |
| | | X511/X5143/X5150/X5152/X5153/X5155/X6347/X6350/X6351/X7518/X854/ |
| | | X858 |
| ASN_struct_aa_Q | −2.33 | X10425/X10426/X10427/X10428/X10429/X11095/X11096/X11581/X1162/X1163/ |
| | | X1171/X1831/X1834/X1836/X1842/X1844/X2699/X2700/X2702/X2707/X2709/ |
| | | X2712/X2716/X3072/X3742/X3743/X3744/X3746/X3749/X3753/X3761/ |
| | | X3763/X383/X4123/X4130/X4915/X4916/X4919/X4924/X4926/X4927/X4929/ |
| | | X4940/X5276/X5282/X6160/X6165/X6167/X6170/X6171/X6173/X6174/X6175/ |
| | | X6191/X6461/X6476/X697/X7380/X7381/X7382/X7384/X7385/X7390/ |
| | | X7392/X7393/X7394/X7395/X7401/X7617/X8542/X8544/X8545/X8546/X8548/ |
| | | X8550/X8551/X8552/X8553/X8558/X9566/X9567/X9568/X9569/X9571/X9573/ |
| | | X9574/X9575 |
| ASN_struct_aa_R | −2.33 | X1362/X1363/X1364/X18/X2060/X2065/X2068/X265/X29/X2948/X2961/X3072/ |
| | | X4003/X4123/X4132/X512/X5278/X5282/X5289/X6461/X6470/X6478/X69/ |
| | | X7620/X7639/X857/X859/X8714/X90 |
| ASN_strucl_aa_S | −2.33 | X117/X13/X1354/X1479/X2212/X263/X45/X508/X509/X851/X852 |
| ASN_struct_aa_T | −2.33 | X1993/X2885/X3938 |
| ASN_struct_aa_V | −2.33 | X10426/X393/X4923/X4924/X6164/X6165/X6171/X7380/X7385/X7389/X7390/ |
| | | X8542/X8548/X8553/X9569/X9571 |
| ASN_struct_SS_dsspE | −2.33 | X1207/X18/X1883/X3947/X729 |
| ASN_struct_SS_dsspS | −2.33 | X1277/X1281/X1287/X1974/X1978/X239/X2868/X4132/X462/X5278/X5289/ |
| | | X6470/X6478/X7620/X7639/X787/X793/X8714 |
| ASN_struct_SS_dsspT | −2.33 | X191/X74 |
| SER.THR_seq_aaAll_P | −2.33 | X119/X1327/X1343/X1405/X1407/X149/X206/X2101/X211/X2111/X214/X221/ |
| | | X27/X2983/X32/X380/X381/X412/X423/X438/X456/X501/X6/X771/X781/ |
| | | X782/X84/X842/X88/X891/X90/X93 |
| SER.THR_seq_aaAll_R | −2.33 | X237/X495/X68 |
| SER.THR_seq_aaAll_S | −2.33 | X16/X207/X208/X271/X415/X418/X589/X85/X86/X95 |
| SER.THR_seq_aaDown_P | −2.33 | X119/X149/X211/X27/X32/X380/X456/X6/X781/X90/X93 |
| SER.THR_struct_aa_P | −2.33 | X119/X1327/X1343/X1405/X1407/X149/X206/X2101/X211/X2111/X214/X221/ |
| | | X27/X2983/X32/X380/X381/X412/X423/X438/X456/X501/X6/X771/X781/ |
| | | X782/X84/X842/X88/X891/X90/X93 |
| ASN_seq_aaAll_D | −3.09 | X74 |
| ASN_seq_aaAll_E | −3.09 | X170 |
| ASN_seq_aaAll_F | −3.09 | X4132/X5278/X5289/X6470/X6478/X7620/X7639/X8714 |
| ASN_seq_aaAll_K | −3.09 | X1267/X1953/X2828/X3865 |
| ASN_seq_aaAll_N | −3.09 | X1199/X1202/X1204/X1206/X1207/X1266/X1267/X1876/X1878/X1883/X1952/ |
| | | X1953/X197/X198/X24/X25/X2751/X2827/X2828/X3865/X403/X406/X407/ |
| | | X722/X725/X727/X728/X729/X77/X78 |
| ASN_seq_aaAll_P | −3.09 | X1416/X170/X2123/X3006 |
| ASN_seq_aaAll_R | −3.09 | X4130/X4132/X5276/X5278/X5289/X6470/X6476/X6478/X7617/X7620/X7639/ |
| | | X8714 |
| ASN_seq_aaAll_T | −3.09 | X5096 |
| ASN_seq_aaAll_V | −3.09 | X1317/X1320/X1321/X1322/X2012/X2016/X2017/X2021/X2022/X2023/X253/ |
| | | X2896/X2900/X2901/X2910/X2913/X2914/X3940/X3954/X3955/X3958/X3959/ |
| | | X487/X5103/X5107/X5108/X6314/X823/X824 |
| ASN_seq_aaDown_E | −3.09 | X1358/X1361/X1362/X1363/X1364/X1365/X2056/X2060/X2062/X2064/X2065/ |
| | | X2066/X2067/X2068/X2069/X265/X29/X2948/X2950/X2952/X2955/X2956/ |
| | | X2958/X2959/X2960/X2961/X2962/X3994/X3997/X4000/X4001/X4003/X4005/ |
| | | X4007/X4008/X4009/X511/X512/X5143/X5148/X5150/X5152/X5153/X5155/ |
| | | X6347/X6350/X6351/X7518/X854/X857/X858/X859/X90 |
| ASN_seq_aaDown_F | −3.09 | X24/X77 |
| ASN_seq_aaDown_K | −3.09 | X2718/X3755/X4923/X4942/X6164/X7389 |
| ASN_seq_aaDown_Q | −3.09 | X1328/X2083/X829 |
| ASN_seq_aaDown_R | −3.09 | X1219/X1224/X1226/X1240/X1899/X1908/X191/X1914/X1919/X216/X26/X2771/ |
| | | X2777/X2799/X3829/X3833/X424/X427/X4982/X7/X74/X744/X749/X752/X92 |
| ASN_seq_aaDown_T | −3.09 | X170/X3938/X5096 |
| ASN_seq_aaDown_Y | −3.09 | X1219/X1240/X1899/X1914/X1919/X2771/X2777/X2799/X3829/X3833/X4982 |
| ASN_seq_aaUp_A | −3.09 | X18 |
| ASN_seq_aaUp_D | −3.09 | X2064/X2959/X4007/X5148 |
| ASN_seq_aaUp_E | −3.09 | X1219/X1899/X2771 |
| ASN_seq_aaUp_F | −3.09 | X18 |
| ASN_seq_SS_sspro8C | −3.09 | X1416/X1951/X2123/X2826/X3006/X3864 |
| ASN_seq_SS_sspro8E | −3.09 | X10426/X1219/X1224/X1226/X1899/X1908/X216/X26/X2718/X2771/X3755/ |
| | | X3768/X424/X427/X4923/X4934/X4942/X6164/X6176/X6186/X7/X7389/X7396/ |
| | | X7397/X744/X749/X752/X8547/X8548/X8555/X8556/X92/X9570/X9571 |
| ASN_seq_SS_sspro8S | −3.09 | X2718/X3755/X3768/X3769/X4923/X4934/X4935/X4942/X6164/X6176/X6186/ |
| | | X6187/X7389/X7396 |
| ASN_seq_SS_ssproE | −3.09 | X1219/X1224/X1226/X1899/X1908/X216/X26/X2700/X2718/X2771/X3749/X3755/ |
| | | X3768/X424/X427/X4923/X4927/X4934/X4942/X6164/X6176/X6186/X7/ |
| | | X7389/X7396/X7397/X744/X749/X752/X8547/X8555/X8556/X92/X9570 |
| ASN_struct_aa_A | −3.09 | X1274/X1277/X1281/X1287/X1289/X1316/X1318/X1416/X1971/X1974/X1976/ |
| | | X1978/X1984/X2011/X2013/X2015/X2017/X2123/X239/X2857/X2861/X2868/ |
| | | X2870/X2895/X2899/X2901/X2904/X3006/X3922/X3946/X3953/X3955/X462/ |
| | | X485/X5106/X5108/X787/X793/X822 |
| ASN_struct_aa_D | −3.09 | X1357/X1360/X2058/X2059/X2061/X214/X2945/X2949/X2954/X3991/X4004/ |
| | | X510/X5146/X84/X856 |
| ASN_struct_aa_M | −3.09 | X10425/X10426/X11095/X1219/X1899/X2771/X3769/X4924/X4935/X4947/X4948/ |
| | | X6165/X6170/X6171/X6181/X6182/X6187/X6191/X7380/X7384/X7385/ |
| | | X7390/X7392/X7396/X7397/X7401/X7403/X7404/X8542/X8547/X8548/X8550/ |
| | | X8552/X8553/X8555/X8556/X8558/X9566/X9569/X9570/X9571/X9573 |
| ASN_struct_aa_N | −3.09 | X1199/X1202/X1204/X1206/X1207/X1266/X1267/X1876/X1878/X1883/X1952/ |
| | | X1953/X197/X198/X24/X25/X2751/X2827/X2828/X3865/X403/X406/X407/ |
| | | X722/X725/X727/X728/X729/X77/X78 |
| ASN_struct_aa_Q | −3.09 | X2718/X3755/X3768/X3769/X4923/X4934/X4935/X4942/X4947/X4948/X6164/ |
| | | X6176/X6181/X6182/X6186/X6187/X7389/X7396/X7397/X7403/X7404/X8547/ |
| | | X8555/X8556/X9570 |
| ASN_struct_aa_R | −3.09 | X1357/X1358/X1360/X1361/X1365/X2056/X2058/X2059/X2061/X2062/X2064/ |
| | | X2066/X2067/X2069/X214/X2945/X2949/X2950/X2952/X2954/X2955/X2956/ |
| | | X2958/X2959/X2960/X2962/X3991/X3994/X3997/X4000/X4001/X4004/X4005/ |
| | | X4007/X4008/X4009/X510/X511/X5143/X5146/X5148/X5150/X5152/X5153/ |
| | | X5155/X6347/X6350/X6351/X7518/X84/X854/X856/X858 |
| ASN_struct_aa_S | −3.09 | X170 |
| ASN_struct_aa_T | −3.09 | X170 |
| ASN_struct_SS_dsspE | −3.09 | X1266/X1952/X2827 |
| ASN_struct_SS_dsspS | −3.09 | X1358/X1361/X1362/X1363/X1364/X1365/X2056/X2060/X2062/X2064/X2065/ |
| | | X2066/X2067/X2068/X2069/X265/X29/X2948/X2950/X2952/X2955/X2956/ |
| | | X2958/X2959/X2960/X2961/X2962/X3072/X3994/X3997/X4000/X4001/X4003/ |
| | | X4005/X4007/X4008/X4009/X4123/X511/X512/X5143/X5148/X5150/X5152/ |
| | | X5153/X5155/X5282/X6347/X6350/X6351/X6461/X7518/X854/X857/X858/ |
| | | X859/X90 |
| ASN_seq_aaAll_N | −3.72 | X1950/X1951/X2825/X2826/X3863/X3864/X5318 |
| ASN_seq_aaAll_T | −3.72 | X1993/X2885/X3938 |
| ASN_seq_aaAll_V | −3.72 | X1316/X1318/X2011/X2013/X2015/X2895/X2899/X2904/X3946/X3953/X485/ |
| | | X5106/X822 |
| ASN_seq_aaDown_T | −3.72 | X1993/X2885 |
| ASN_seq_aaDown_Y | −3.72 | X1224/X1226/X1908/X216/X26/X424/X427/X7/X744/X749/X752/X92 |
| ASN_seq_aaUp_E | −3.72 | X1224/X1226/X1908/X216/X26/X3833/X424/X427/X4982/X7/X744/X749/X752/X92 |
| ASN_seq_SS_sspro8C | −3.72 | X1199/X1202/X1204/X1876/X1878/X197/X24/X2751/X403/X722/X725/X77 |
| ASN_seq_SS_sspro8S | −3.72 | X1219/X1899/X2771 |
| ASN_struct_aa_M | −3.72 | X1224/X1226/X1908/X216/X26/X2718/X3755/X3768/X424/X427/X4923/X4934/ |
| | | X4942/X6164/X6176/X6186/X7/X7389/X744/X749/X752/X92 |
| ASN_struct_aa_N | −3.72 | X1950/X1951/X2825/X2826/X3863/X3864/X5318 |
| ASN_struct_aa_Q | −3.72 | X1219/X1224/X1226/X1899/X1908/X216/X26/X2771/X3833/X424/X427/X4982/ |
| | | X7/X744/X749/X752/X92 |
| ASN_struct_aa_V | −3.72 | X1219/X1224/X1226/X1899/X1908/X216/X26/X2771/X424/X427/X7/X744/X749/ |
| | | X752/X92 |
| ASN_seq_aaUp_E | −4.26 | X1240/X1914/X1919/X2777/X2799/X3829 |
| ASN_seq_SS_sspro8S | −4.26 | X1224/X1226/X1908/X216/X26/X424/X427/X7/X744/X749/X752/X92 |
| ASN_struct_aa_Q | −4.26 | X1240/X1914/X1919/X2777/X2799/X3829 |
| ASN_seq_aaAll_C | −Inf | X2018/X2024/X2902/X2906/X2908/X2912/X3244/X3949/X3951/X3956/X3960/ |
| | | X4315/X5109/X5111/X5113/X5115/X5499/X6320/X6322/X6693 |
| ASN_seq_aaAll_E | −Inf | X1294/X1295/X1296/X1993/X1995/X1996/X245/X27/X2885/X2887/X356/X3938/ |
| | | X470/X80/X801/X802 |
| ASN_seq_aaAll_F | −Inf | X10462/X10463/X10465/X10466/X10469/X10470/X10471/X10472/X10473/X10474/ |
| | | X10475/X10477/X10478/X10479/X10480/X10481/X10483/X10484/X10485/ |
| | | X10486/X10487/X10488/X10489/X10491/X10492/X10493/X10495/X10497/ |
| | | X10498/X10500/X10502/X10504/X10506/X10507/X10509/X10512/X10514/ |
| | | X10517/X10519/X10524/X11108/X11109/X11112/X11114/X11117/X11118/ |
| | | X11119/X11120/X11121/X11122/X11123/X11125/X11126/X11127/X11128/ |
| | | X11129/X11130/X11131/X11132/X11135/X11136/X11138/X11140/X11142/ |
| | | X11144/X11147/X11585/X11587/X11590/X11593/X11594/X11595/X11596/X11597/ |
| | | X11599/X11600/X11601/X11603/X11605/X11898/X11901/X11904/X11905/ |
| | | X11906/X12091/X1439/X2161/X2163/X3063/X3064/X3066/X3074/X4115/ |
| | | X4116/X4117/X4118/X4125/X4129/X4131/X4133/X4134/X5262/X5263/X5265/ |
| | | X5266/X5267/X5268/X5269/X5270/X5275/X5277/X5279/X5280/X5284/ |
| | | X5285/X5286/X5287/X5288/X5290/X5292/X6445/X6446/X6447/X6448/X6449/ |
| | | X6450/X6451/X6453/X6454/X6455/X6456/X6457/X6458/X6459/X6465/X6466/ |
| | | X6467/X6468/X6469/X6471/X6473/X6475/X6477/X6479/X6480/X6481/ |
| | | X6482/X6483/X6484/X6485/X6488/X6489/X6519/X7591/X7592/X7594/X7595/ |
| | | X7596/X7597/X7598/X7599/X7600/X7601/X7602/X7603/X7604/X7605/X7606/ |
| | | X7607/X7608/X7609/X7610/X7611/X7612/X7616/X7619/X7623/X7625/ |
| | | X7626/X7627/X7628/X7629/X7630/X7633/X7634/X7635/X7636/X7637/X7638/ |
| | | X7640/X7642/X7643/X7645/X7646/X7648/X7650/X7666/X7669/X7677/X8676/ |
| | | X8678/X8679/X8680/X8681/X8682/X8683/X8684/X8685/X8686/X8687/ |
| | | X8688/X8689/X8690/X8692/X8693/X8694/X8695/X8696/X8697/X8698/X8699/ |
| | | X8700/X8701/X8702/X8703/X8704/X8705/X8708/X8710/X8711/X8713/X8717/ |
| | | X8719/X8720/X8722/X8723/X8725/X8727/X8728/X8729/X8730/X8731/ |
| | | X8732/X8735/X8736/X8738/X8739/X8741/X8749/X8751/X8755/X8760/X8766/ |
| | | X9639/X9640/X9641/X9643/X9644/X9645/X9646/X9647/X9648/X9649/X9650/ |
| | | X9651/X9652/X9653/X9654/X9655/X9656/X9658/X9659/X9660/X9661/ |
| | | X9662/X9663/X9664/X9666/X9667/X9668/X9669/X9670/X9673/X9675/X9676/ |
| | | X9678/X9679/X9682/X9683/X9685/X9686/X9688/X9690/X9692/X9693/X9695/ |
| | | X9697/X9699/X9703/X9705/X9707/X9710/X9716/X9720 |
| ASN_seq_aaAll_G | −Inf | X356 |
| ASN_seq_aaAll_H | −Inf | X10462/X10463/X10465/X10466/X10469/X10470/X10471/X10472/X10473/X10474/ |
| | | X10475/X10477/X10478/X10479/X10480/X10481/X10483/X10484/X10485/ |
| | | X10486/X10487/X10488/X10489/X10491/X10492/X10493/X10494/X10495/ |
| | | X10496/X10497/X10498/X10499/X10500/X10501/X10502/X10503/X10504/ |
| | | X10505/X10506/X10507/X10508/X10509/X10510/X10512/X10514/X10517/ |
| | | X10519/X10524/X11108/X11109/X11112/X11114/X11117/X11118/X11119/ |
| | | X11120/X11121/X11122/X11123/X11125/X11126/X11127/X11128/X11129/ |
| | | X11130/X11131/X11132/X11134/X11135/X11136/X11137/X11138/X11139/X11140/ |
| | | X11142/X11144/X11147/X11585/X11587/X11590/X11593/X11594/X11595/ |
| | | X11596/X11597/X11599/X11600/X11601/X11602/X11603/X11605/X11898/ |
| | | X11901/X11904/X11905/X11906/X12091/X1356/X1357/X1359/X1360/X1439/ |
| | | X1440/X1441/X2055/X2057/X2058/X2059/X2061/X2063/X2120/X214/ |
| | | X2161/X2162/X2163/X2164/X2165/X2166/X254/X2944/X2945/X2946/X2947/ |
| | | X2949/X2951/X2953/X2954/X2957/X2996/X2997/X3002/X3004/X3063/X3064/ |
| | | X3065/X3066/X3068/X3069/X3070/X3071/X3073/X3074/X3075/X3991/X3992/ |
| | | X3993/X3995/X3996/X3998/X3999/X4002/X4004/X4006/X4025/X4026/ |
| | | X4032/X4033/X4037/X4038/X4039/X4040/X4042/X4114/X4115/X4116/X4117/ |
| | | X4118/X4119/X4122/X4124/X4125/X4126/X4127/X4128/X4129/X4130/X4131/ |
| | | X4133/X4134/X4136/X4137/X4138/X489/X510/X5141/X5142/X5144/X5145/ |
| | | X5146/X5147/X5149/X5151/X5154/X5161/X5164/X5165/X5169/X5170/ |
| | | X5171/X5172/X5173/X5174/X5178/X5179/X5180/X5181/X5262/X5263/X5264/ |
| | | X5265/X5266/X5267/X5268/X5269/X5270/X5272/X5273/X5274/X5275/X5276/ |
| | | X5277/X5279/X5280/X5281/X5283/X5284/X5285/X5286/X5287/X5288/ |
| | | X5290/X5291/X5292/X5293/X5297/X5298/X5299/X5300/X5301/X5302/X6344/ |
| | | X6345/X6346/X6348/X6349/X6352/X6353/X6355/X6356/X6357/X6358/X6359/ |
| | | X6363/X6364/X6365/X6366/X6367/X6368/X6369/X6445/X6446/X6447/ |
| | | X6448/X6449/X6450/X6451/X6452/X6453/X6454/X6455/X6456/X6457/X6458/ |
| | | X6459/X6460/X6462/X6463/X6465/X6466/X6467/X6468/X6469/X6471/X6472/ |
| | | X6473/X6474/X6475/X6476/X6477/X6479/X6480/X6481/X6482/X6483/ |
| | | X6484/X6485/X6486/X6487/X6488/X6489/X6490/X6491/X6495/X6496/X6497/ |
| | | X6498/X6499/X6500/X6501/X6502/X6519/X7517/X7519/X7520/X7521/X7522/ |
| | | X7524/X7525/X7526/X7527/X7528/X7529/X7530/X7591/X7592/X7593/ |
| | | X7594/X7595/X7596/X7597/X7598/X7599/X7600/X7601/X7602/X7603/X7604/ |
| | | X7605/X7606/X7607/X7608/X7609/X7610/X7611/X7612/X7614/X7615/X7616/ |
| | | X7617/X7618/X7619/X7621/X7623/X7625/X7626/X7627/X7628/X7629/ |
| | | X7630/X7631/X7632/X7633/X7634/X7635/X7636/X7637/X7638/X7640/X7641/ |
| | | X7642/X7643/X7644/X7645/X7646/X7647/X7648/X7649/X7650/X7651/X7652/ |
| | | X7654/X7655/X7656/X7657/X7658/X7659/X7660/X7661/X7666/X7669/ |
| | | X7677/X84/X855/X856/X8633/X8634/X8635/X8636/X8676/X8678/X8679/X8680/ |
| | | X8681/X8682/X8683/X8684/X8685/X8686/X8687/X8688/X8689/X8690/ |
| | | X8692/X8693/X8694/X8695/X8696/X8697/X8698/X8699/X8700/X8701/X8702/ |
| | | X8703/X8704/X8705/X8707/X8708/X8709/X8710/X8711/X8712/X8713/X8715/ |
| | | X8716/X8717/X8718/X8719/X8720/X8721/X8722/X8723/X8724/X8725/ |
| | | X8726/X8727/X8728/X8729/X8730/X8731/X8732/X8733/X8734/X8735/X8736/ |
| | | X8737/X8738/X8739/X8740/X8741/X8742/X8743/X8744/X8745/X8746/X8747/ |
| | | X8748/X8749/X8751/X8755/X8760/X8766/X907/X9639/X9640/X9641/ |
| | | X9643/X9644/X9645/X9646/X9647/X9648/X9649/X9650/X9651/X9652/X9653/ |
| | | X9654/X9655/X9656/X9658/X9659/X9660/X9661/X9662/X9663/X9664/X9666/ |
| | | X9667/X9668/X9669/X9670/X9672/X9673/X9674/X9675/X9676/X9677/ |
| | | X9678/X9679/X9680/X9681/X9682/X9683/X9684/X9685/X9686/X9687/X9688/ |
| | | X9689/X9690/X9691/X9692/X9693/X9694/X9695/X9696/X9697/X9698/X9699/ |
| | | X9700/X9701/X9702/X9703/X9705/X9707/X9710/X9716/X9720 |
| ASN_seq_aaAll_K | −Inf | X1316/X1317/X1318/X1320/X1321/X1322/X1950/X2011/X2012/X2013/X2015/ |
| | | X2016/X2017/X2021/X2022/X2023/X253/X2825/X2895/X2896/X2899/X2900/ |
| | | X2901/X2904/X2910/X2913/X2914/X3863/X3940/X3946/X3953/X3954/X3955/ |
| | | X3958/X3959/X485/X487/X5103/X5106/X5107/X5108/X6314/X822/X823/X824 |
| ASN_seq_aaAll_L | −Inf | X15 |
| ASN_seq_aaAll_M | −Inf | X10494/X1356/X1359/X2055/X2057/X2063/X2120/X2944/X2946/X2951/X2953/ |
| | | X2957/X2996/X2997/X3002/X3004/X3992/X3993/X3995/X3996/X3998/X4002/ |
| | | X4006/X4025/X4026/X4032/X4033/X4037/X4038/X4039/X4040/X4042/ |
| | | X5141/X5142/X5144/X5145/X5147/X5151/X5154/X5161/X5164/X5165/X5169/ |
| | | X5170/X5171/X5172/X5173/X5174/X5178/X5179/X5180/X5181/X5293/X6344/ |
| | | X6346/X6348/X6349/X6352/X6353/X6355/X6356/X6357/X6358/X6359/ |
| | | X6363/X6364/X6365/X6366/X6367/X6368/X6369/X6462/X6490/X6491/X7517/ |
| | | X7519/X7520/X7521/X7522/X7524/X7525/X7526/X7527/X7528/X7529/X7530/ |
| | | X7615/X7618/X7621/X7651/X7652/X855/X8633/X8634/X8635/X8636/ |
| | | X8707/X8709/X8712/X8715/X8716/X8742/X9672/X9674/X9677/X9689 |
| ASN_seq_aaAll_P | −Inf | X1527/X1528/X1529/X2283/X2285/X238/X3207/X356/X568/X965/X966 |
| ASN_seq_aaAll_Q | −Inf | X1294/X1295/X170/X1996/X245/X27/X470/X80/X801/X802 |
| ASN_seq_aaAll_R | −Inf | X10462/X10463/X10465/X10469/X10470/X10471/X10472/X10473/X10474/X10483/ |
| | | X10484/X10485/X10495/X11108/X11112/X11117/X11118/X11119/X11585/ |
| | | X1439/X2161/X2163/X3064/X3066/X3074/X4116/X4117/X4125/X4129/ |
| | | X4131/X4133/X4134/X5262/X5265/X5267/X5268/X5269/X5270/X5275/X5277/ |
| | | X5279/X5280/X5284/X5285/X5286/X5287/X5288/X5290/X6445/X6447/X6448/ |
| | | X6449/X6450/X6453/X6454/X6455/X6456/X6457/X6458/X6459/X6465/ |
| | | X6466/X6467/X6468/X6469/X6471/X6475/X6477/X6479/X6480/X6481/X6482/ |
| | | X6483/X6484/X7591/X7594/X7595/X7596/X7597/X7598/X7599/X7600/X7602/ |
| | | X7603/X7604/X7605/X7606/X7607/X7608/X7609/X7610/X7611/X7616/ |
| | | X7619/X7623/X7625/X7626/X7627/X7628/X7629/X7635/X7636/X7637/X7638/ |
| | | X7640/X7643/X8676/X8678/X8680/X8681/X8682/X8683/X8684/X8685/X8686/ |
| | | X8687/X8688/X8689/X8692/X8693/X8694/X8695/X8696/X8697/X8698/ |
| | | X8699/X8700/X8701/X8708/X8710/X8711/X8713/X8717/X8720/X8728/X8729/ |
| | | X8730/X8731/X9639/X9640/X9641/X9643/X9644/X9645/X9646/X9647/X9648/ |
| | | X9649/X9650/X9651/X9652/X9658/X9659/X9660/X9661/X9662/X9663/ |
| | | X9673/X9675/X9676/X9678/X9690 |
| ASN_seq_aaAll_S | −Inf | X74 |
| ASN_seq_aaAll_T | −Inf | X1333/X1334/X1335/X1336/X1337/X2028/X2029/X2031/X2032/X2033/X2034/ |
| | | X210/X256/X2916/X2918/X2919/X2920/X2921/X31/X356/X3962/X3964/X3965/ |
| | | X496/X497/X5118/X835/X836/X837/X89/X9/X91 |
| ASN_seq_aaAll_V | −Inf | X2018/X2024/X2902/X2906/X2907/X2908/X2912/X3244/X3435/X3943/X3949/ |
| | | X3950/X3951/X3956/X3960/X4315/X4316/X4544/X5100/X5109/X5111/X5112/ |
| | | X5113/X5115/X5498/X5499/X5756/X6317/X6320/X6321/X6322/X6693/ |
| | | X6694/X6972/X7503/X7847 |
| ASN_seq_aaAll_Y | −Inf | X4130/X5276/X5287/X6468/X6476/X6483/X7617/X7628/X7637/X8711/X8730/ |
| | | X9676 |
| ASN_seq_aaDown_A | −Inf | X10462/X10463/X10465/X10466/X10469/X10470/X10471/X10472/X10473/X10474/ |
| | | X10475/X10477/X10478/X10479/X10480/X10481/X10483/X10484/X10485/ |
| | | X10486/X10487/X10488/X10489/X10491/X10492/X10493/X10494/X10495/ |
| | | X10496/X10497/X10498/X10499/X10500/X10501/X10502/X10503/X10504/ |
| | | X10505/X10506/X10507/X10508/X10509/X10510/X10512/X10514/X10517/ |
| | | X10519/X10524/X11108/X11109/X11112/X11114/X11117/X11118/X11119/ |
| | | X11120/X11121/X11122/X11123/X11125/X11126/X11127/X11128/X11129/ |
| | | X11130/X11131/X11132/X11134/X11135/X11136/X11137/X11138/X11139/X11140/ |
| | | X11142/X11144/X11147/X11585/X11587/X11590/X11593/X11594/X11595/ |
| | | X11596/X11597/X11599/X11600/X11601/X11602/X11603/X11605/X11898/ |
| | | X11901/X11904/X11905/X11906/X12091/X1356/X1357/X1359/X1360/X1439/ |
| | | X1440/X1441/X170/X2055/X2057/X2058/X2059/X2061/X2063/X2120/ |
| | | X214/X2161/X2162/X2163/X2164/X2165/X2166/X254/X2944/X2945/X2946/ |
| | | X2947/X2949/X2951/X2953/X2954/X2957/X2996/X2997/X3002/X3004/X3063/ |
| | | X3064/X3065/X3066/X3068/X3069/X3070/X3071/X3073/X3074/X3075/X356/ |
| | | X3991/X3992/X3993/X3995/X3996/X3998/X3999/X4002/X4004/X4006/X4025/ |
| | | X4026/X4032/X4033/X4037/X4038/X4039/X4040/X4042/X4114/X4115/ |
| | | X4116/X4117/X4118/X4119/X4122/X4124/X4125/X4126/X4127/X4128/X4129/ |
| | | X4130/X4131/X4133/X4134/X4136/X4137/X4138/X489/X510/X5141/X5142/ |
| | | X5144/X5145/X5146/X5147/X5149/X5151/X5154/X5161/X5164/X5165/X5169/ |
| | | X5170/X5171/X5172/X5173/X5174/X5178/X5179/X5180/X5181/X5262/ |
| | | X5263/X5264/X5265/X5266/X5267/X5268/X5269/X5270/X5272/X5273/X5274/ |
| | | X5275/X5276/X5277/X5279/X5280/X5281/X5283/X5284/X5285/X5286/X5287/ |
| | | X5288/X5290/X5291/X5292/X5293/X5297/X5298/X5299/X5300/X5301/ |
| | | X5302/X6344/X6345/X6346/X6348/X6349/X6352/X6353/X6355/X6356/X6357/ |
| | | X6358/X6359/X6363/X6364/X6365/X6366/X6367/X6368/X6369/X6445/X6446/ |
| | | X6447/X6448/X6449/X6450/X6451/X6452/X6453/X6454/X6455/X6456/ |
| | | X6457/X6458/X6459/X6460/X6462/X6463/X6465/X6466/X6467/X6468/X6469/ |
| | | X6471/X6472/X6473/X6474/X6475/X6476/X6477/X6479/X6480/X6481/X6482/ |
| | | X6483/X6484/X6485/X6486/X6487/X6488/X6489/X6490/X6491/X6495/ |
| | | X6496/X6497/X6498/X6499/X6500/X6501/X6502/X6519/X7517/X7519/X7520/ |
| | | X7521/X7522/X7524/X7525/X7526/X7527/X7528/X7529/X7530/X7591/X7592/ |
| | | X7593/X7594/X7595/X7596/X7597/X7598/X7599/X7600/X7601/X7602/ |
| | | X7603/X7604/X7605/X7606/X7607/X7608/X7609/X7610/X7611/X7612/X7614/ |
| | | X7615/X7616/X7617/X7618/X7619/X7621/X7623/X7625/X7626/X7627/X7628/ |
| | | X7629/X7630/X7631/X7632/X7633/X7634/X7635/X7636/X7637/X7638/ |
| | | X7640/X7641/X7642/X7643/X7644/X7645/X7646/X7647/X7648/X7649/X7650/ |
| | | X7651/X7652/X7654/X7655/X7656/X7657/X7658/X7659/X7660/X7661/X7666/ |
| | | X7669/X7677/X84/X855/X856/X8633/X8634/X8635/X8636/X8676/X8678/ |
| | | X8679/X8680/X8681/X8682/X8683/X8684/X8685/X8686/X8687/X8688/X8689/ |
| | | X8690/X8692/X8693/X8694/X8695/X8696/X8697/X8698/X8699/X8700/ |
| | | X8701/X8702/X8703/X8704/X8705/X8707/X8708/X8709/X8710/X8711/X8712/ |
| | | X8713/X8715/X8716/X8717/X8718/X8719/X8720/X8721/X8722/X8723/X8724/ |
| | | X8725/X8726/X8727/X8728/X8729/X8730/X8731/X8732/X8733/X8734/ |
| | | X8735/X8736/X8737/X8738/X8739/X8740/X8741/X8742/X8743/X8744/X8745/ |
| | | X8746/X8747/X8748/X8749/X8751/X8755/X8760/X8766/X907/X9639/X9640/ |
| | | X9641/X9643/X9644/X9645/X9646/X9647/X9648/X9649/X9650/X9651/ |
| | | X9652/X9653/X9654/X9655/X9656/X9658/X9659/X9660/X9661/X9662/X9663/ |
| | | X9664/X9666/X9667/X9668/X9669/X9670/X9672/X9673/X9674/X9675/X9676/ |
| | | X9677/X9678/X9679/X9680/X9681/X9682/X9683/X9684/X9685/X9686/ |
| | | X9687/X9688/X9689/X9690/X9691/X9692/X9693/X9694/X9695/X9696/X9697/ |
| | | X9698/X9699/X9700/X9701/X9702/X9703/X9705/X9707/X9710/X9716/X9720 |
| ASN_seq_aaDown_C | −Inf | X1316/X1317/X1318/X1320/X1321/X1322/X2011/X2012/X2013/X2015/X2016/ |
| | | X2017/X2021/X2022/X2023/X253/X2895/X2896/X2899/X2900/X2901/X2904/ |
| | | X2910/X2913/X2914/X3940/X3946/X3953/X3954/X3955/X3958/X3959/X485/ |
| | | X487/X5103/X5106/X5107/X5108/X6314/X822/X823/X824 |
| ASN_seq_aaDown_E | −Inf | X10469/X10472/X10473/X10483/X10494/X10495/X11117/X1356/X1357/X1359/ |
| | | X1360/X1439/X1440/X2055/X2057/X2058/X2059/X2061/X2063/X2120/X214/ |
| | | X2161/X2162/X2163/X2166/X254/X2944/X2945/X2946/X2947/X2949/X2951/ |
| | | X2953/X2954/X2957/X2996/X2997/X3002/X3004/X3064/X3065/X3066/ |
| | | X3070/X3073/X3074/X3991/X3992/X3993/X3995/X3996/X3998/X3999/X4002/ |
| | | X4004/X4006/X4025/X4026/X4032/X4033/X4037/X4038/X4039/X4040/X4042/ |
| | | X4114/X4116/X4117/X4119/X4124/X4125/X4127/X4129/X4130/X4133/ |
| | | X4134/X489/X510/X5141/X5142/X5144/X5145/X5146/X5147/X5149/X5151/ |
| | | X5154/X5161/X5164/X5165/X5169/X5170/X5171/X5172/X5173/X5174/X5178/ |
| | | X5179/X5180/X5181/X5262/X5264/X5265/X5267/X5268/X5270/X5273/X5275/ |
| | | X5276/X5279/X5280/X5283/X5284/X5285/X5286/X5287/X5290/X5293/ |
| | | X6344/X6345/X6346/X6348/X6349/X6352/X6353/X6355/X6356/X6357/X6358/ |
| | | X6359/X6363/X6364/X6365/X6366/X6367/X6368/X6369/X6445/X6447/X6448/ |
| | | X6450/X6452/X6453/X6456/X6457/X6459/X6462/X6463/X6465/X6466/ |
| | | X6467/X6468/X6471/X6475/X6476/X6479/X6480/X6481/X6482/X6483/X6490/ |
| | | X6491/X7517/X7519/X7520/X7521/X7522/X7524/X7525/X7526/X7527/X7528/ |
| | | X7529/X7530/X7593/X7594/X7597/X7598/X7600/X7602/X7603/X7605/ |
| | | X7606/X7609/X7610/X7615/X7616/X7617/X7618/X7621/X7623/X7625/X7626/ |
| | | X7627/X7628/X7635/X7636/X7637/X7640/X7643/X7651/X7652/X84/X855/ |
| | | X856/X8633/X8634/X8635/X8636/X8680/X8681/X8683/X8684/X8687/X8688/ |
| | | X8692/X8695/X8696/X8698/X8699/X8707/X8708/X8709/X8710/X8711/X8712/ |
| | | X8715/X8716/X8717/X8720/X8728/X8729/X8730/X8742/X907/X9643/ |
| | | X9646/X9647/X9649/X9650/X9658/X9661/X9662/X9672/X9673/X9674/X9675/ |
| | | X9676/X9677/X9689/X9690 |
| ASN_seq_aaDown_F | −Inf | X10425/X10426/X10427/X10428/X10429/X10459/X10494/X11095/X11096/X11581/ |
| | | X117/X1219/X1220/X1221/X1222/X1223/X1224/X1225/X1226/X1227/ |
| | | X1228/X1229/X1230/X1231/X1232/X1233/X1234/X1235/X1236/X1237/X1238/ |
| | | X1239/X1240/X13/X1354/X1355/X1356/X1358/X1359/X1361/X1365/X1431/ |
| | | X1432/X1433/X1435/X1437/X1464/X1479/X1557/X1895/X1896/X1897/X1898/ |
| | | X1899/X1900/X1901/X1902/X1903/X1904/X1905/X1906/X1907/X1908/ |
| | | X1909/X1910/X1911/X1912/X1913/X1914/X1915/X1916/X1917/X1918/X1919/ |
| | | X1920/X1921/X1922/X1923/X1924/X1925/X1951/X2035/X2036/X2054/X2055/ |
| | | X2056/X2057/X2062/X2063/X2064/X2066/X2067/X2069/X2120/X2145/ |
| | | X2146/X2148/X2149/X215/X2150/X2151/X2152/X2154/X2157/X216/X2160/ |
| | | X217/X218/X2186/X2211/X2212/X2331/X2332/X26/X263/X2718/X2764/X2765/ |
| | | X2766/X2767/X2768/X2769/X2770/X2771/X2772/X2773/X2774/X2775/ |
| | | X2776/X2777/X2778/X2779/X2780/X2781/X2782/X2783/X2784/X2785/X2786/ |
| | | X2787/X2788/X2789/X2790/X2791/X2792/X2793/X2794/X2795/X2796/X2797/ |
| | | X2798/X2799/X2826/X2922/X2923/X2924/X2925/X2926/X2927/X2928/ |
| | | X2944/X2946/X2947/X2950/X2951/X2952/X2953/X2955/X2956/X2957/X2958/ |
| | | X2959/X2960/X2962/X2996/X2997/X3002/X3004/X3032/X3033/X3034/X3035/ |
| | | X3036/X3038/X3039/X3040/X3042/X3044/X3045/X3046/X3050/X3051/ |
| | | X3052/X3053/X3056/X3059/X3060/X3061/X3062/X3072/X3115/X3116/X3117/ |
| | | X32/X3285/X3286/X3287/X3293/X3755/X3768/X3769/X3803/X3804/X3805/ |
| | | X3806/X3807/X3808/X3810/X3811/X3812/X3813/X3814/X3815/X3816/X3817/ |
| | | X3818/X3819/X3820/X3821/X3822/X3823/X3824/X3825/X3826/X3827/ |
| | | X3828/X3829/X3830/X3831/X3832/X3833/X3834/X3835/X3836/X3864/X3966/ |
| | | X3967/X3968/X3969/X3970/X3972/X3973/X3974/X3975/X3976/X3977/X3978/ |
| | | X3992/X3993/X3994/X3995/X3996/X3997/X3998/X3999/X4000/X4001/ |
| | | X4002/X4005/X4006/X4007/X4008/X4009/X4025/X4026/X4032/X4033/X4037/ |
| | | X4038/X4039/X4040/X4042/X4066/X4067/X4069/X4070/X4071/X4072/X4073/ |
| | | X4074/X4075/X4076/X4078/X4079/X4080/X4081/X4082/X4083/X4087/ |
| | | X4088/X4089/X4090/X4093/X4095/X4096/X4097/X4098/X4099/X4102/X4103/ |
| | | X4104/X4105/X4108/X4109/X4111/X4112/X4113/X4123/X4130/X4132/X4166/ |
| | | X424/X425/X426/X427/X428/X429/X430/X4373/X4374/X4376/X4378/X4384/ |
| | | X4389/X4392/X4394/X45/X4923/X4924/X4934/X4935/X4942/X4947/X4948/ |
| | | X4969/X4970/X4971/X4973/X4974/X4975/X4976/X4977/X4978/X4979/ |
| | | X4980/X4981/X4982/X4983/X4984/X4985/X4986/X4987/X4988/X4989/X4990/ |
| | | X4991/X508/X509/X511/X5119/X5120/X5123/X5124/X5125/X5127/X5128/ |
| | | X5129/X5130/X5131/X5132/X5133/X5134/X5135/X5141/X5142/X5143/X5144/ |
| | | X5145/X5147/X5148/X5149/X5150/X5151/X5152/X5153/X5154/X5155/ |
| | | X5161/X5164/X5165/X5169/X5170/X5171/X5172/X5173/X5174/X5178/X5179/ |
| | | X5180/X5181/X5200/X5201/X5202/X5203/X5204/X5205/X5206/X5207/X5208/ |
| | | X5210/X5211/X5212/X5213/X5214/X5215/X5217/X5218/X5219/X5220/ |
| | | X5221/X5222/X5225/X5227/X5228/X5229/X5230/X5231/X5232/X5233/X5237/ |
| | | X5238/X5239/X5240/X5241/X5242/X5243/X5244/X5245/X5248/X5249/X5252/ |
| | | X5253/X5254/X5255/X5256/X5257/X5258/X5259/X5260/X5276/X5278/ |
| | | X5282/X5289/X5293/X5309/X5310/X532/X5564/X5565/X5566/X5570/X5575/ |
| | | X5580/X5583/X5586/X5588/X5590/X5594/X6164/X6165/X6170/X6171/X6176/ |
| | | X6181/X6182/X6186/X6187/X6191/X6200/X6201/X6202/X6204/X6205/X6206/ |
| | | X6207/X6208/X6210/X6211/X6325/X6328/X6329/X6330/X6331/X6332/ |
| | | X6334/X6335/X6336/X6337/X6338/X6339/X6344/X6345/X6346/X6347/X6348/ |
| | | X6349/X6350/X6351/X6352/X6353/X6355/X6356/X6357/X6358/X6359/X6363/ |
| | | X6364/X6365/X6366/X6367/X6368/X6369/X6381/X6382/X6383/X6384/ |
| | | X6385/X6386/X6387/X6388/X6389/X6391/X6392/X6393/X6394/X6395/X6396/ |
| | | X6397/X6399/X6400/X6401/X6403/X6404/X6405/X6406/X6407/X6408/X6409/ |
| | | X6413/X6414/X6415/X6416/X6417/X6418/X6419/X6420/X6422/X6424/ |
| | | X6425/X6426/X6427/X6428/X6430/X6431/X6432/X6433/X6434/X6435/X6436/ |
| | | X6437/X6438/X6439/X6440/X6441/X6442/X6443/X6461/X6462/X6470/X6476/ |
| | | X6478/X6490/X6491/X6503/X6506/X6507/X6760/X6762/X6764/X6767/ |
| | | X6772/X6773/X6777/X6787/X6793/X6796/X6798/X6812/X7/X7380/X7384/ |
| | | X7385/X7389/X7390/X7392/X7393/X7396/X7397/X7401/X7403/X7404/X7409/ |
| | | X7412/X744/X745/X746/X747/X748/X749/X750/X7507/X751/X7510/X7511/ |
| | | X7513/X7514/X7515/X7516/X7517/X7518/X7519/X752/X7520/X7521/X7522/ |
| | | X7524/X7525/X7526/X7527/X7528/X7529/X753/X7530/X7537/X7538/X7539/ |
| | | X754/X7540/X7541/X7544/X7545/X7547/X7549/X755/X7550/X7551/X7553/ |
| | | X7554/X7555/X7556/X7557/X7558/X756/X7560/X7561/X7562/X7563/X7564/ |
| | | X7566/X7568/X7570/X7571/X7572/X7573/X7574/X7575/X7576/X7577/ |
| | | X7578/X7579/X7580/X7581/X7582/X7583/X7584/X7585/X7586/X7587/X7589/ |
| | | X7615/X7617/X7618/X7620/X7621/X7639/X7651/X7652/X7662/X7664/X7665/ |
| | | X7912/X7914/X7916/X7922/X7926/X7931/X7946/X7948/X7961/X851/ |
| | | X852/X853/X854/X8542/X8544/X8547/X8548/X855/X8550/X8551/X8552/X8553/ |
| | | X8555/X8556/X8558/X858/X8631/X8632/X8633/X8634/X8635/X8636/X8642/ |
| | | X8645/X8646/X8647/X8648/X8651/X8654/X8655/X8657/X8658/X8659/ |
| | | X8660/X8661/X8664/X8665/X8666/X8667/X8668/X8669/X8670/X8673/X8674/ |
| | | X8707/X8709/X8712/X8714/X8715/X8716/X8742/X8977/X8979/X8990/X9000/ |
| | | X901/X903/X9031/X9041/X9043/X92/X93/X94/X9566/X9568/X9569/X9570/ |
| | | X9571/X9573/X9574/X9575/X9624/X9625/X9630/X9631/X9632/X9634/ |
| | | X9635/X9672/X9674/X9677/X9689/X9903/X9932 |
| ASN_seq_aaDown_H | −Inf | X1357/X1360/X2058/X2059/X2061/X214/X2945/X2947/X2949/X2954/X3991/ |
| | | X3999/X4004/X510/X5146/X5149/X6345/X84/X856 |
| ASN_seq_aaDown_I | −Inf | X1316/X1317/X1318/X1320/X1321/X1322/X2011/X2012/X2013/X2015/X2016/ |
| | | X2017/X2021/X2022/X2023/X253/X2895/X2899/X2900/X2901/X2904/X2910/ |
| | | X2913/X2914/X3946/X3953/X3954/X3955/X3958/X3959/X485/X487/X5106/ |
| | | X5107/X5108/X822/X823/X824 |
| ASN_seq_aaDown_K | −Inf | X1219/X1222/X1223/X1224/X1225/X1226/X1228/X1229/X1230/X1231/X1232/ |
| | | X1233/X1234/X1235/X1236/X1237/X1238/X1239/X1240/X1431/X1435/X1898/ |
| | | X1899/X1901/X1902/X1903/X1904/X1905/X1906/X1907/X1908/X1910/ |
| | | X1911/X1912/X1914/X1915/X1916/X1917/X1918/X1919/X1920/X1921/X1922/ |
| | | X1923/X1924/X1925/X2035/X2146/X2148/X215/X2151/X2154/X216/X217/ |
| | | X218/X26/X2769/X2770/X2771/X2773/X2774/X2775/X2777/X2779/X2780/ |
| | | X2781/X2782/X2783/X2785/X2786/X2789/X2792/X2793/X2794/X2795/X2796/ |
| | | X2797/X2798/X2799/X2922/X2923/X2927/X3036/X3038/X3039/X3042/X3050/ |
| | | X32/X3815/X3816/X3819/X3820/X3823/X3825/X3826/X3827/X3829/X3832/ |
| | | X3833/X3834/X3835/X3836/X3966/X3970/X3972/X3974/X3976/X4070/ |
| | | X4074/X4079/X4087/X4090/X424/X425/X426/X427/X428/X429/X430/X4980/ |
| | | X4982/X4984/X4985/X4988/X5120/X5123/X5125/X5127/X5129/X5134/X5211/ |
| | | X5213/X5237/X532/X6325/X6328/X6334/X6336/X6400/X7/X744/X746/X747/ |
| | | X748/X749/X750/X751/X7510/X752/X753/X754/X755/X756/X901/X92/X93/X94 |
| ASN_seq_aaDown_L | −Inf | X10462/X10463/X10465/X10469/X10470/X10471/X10472/X10473/X10474/X10483/ |
| | | X10484/X10485/X10495/X11108/X11112/X11117/X11118/X11119/X11585/ |
| | | X1439/X1440/X1528/X2161/X2162/X2163/X2166/X254/X3064/X3065/ |
| | | X3066/X3070/X3073/X3074/X4114/X4116/X4117/X4119/X4124/X4125/X4127/ |
| | | X4129/X4130/X4131/X4133/X4134/X489/X5262/X5264/X5265/X5267/X5268/ |
| | | X5269/X5270/X5273/X5275/X5276/X5277/X5279/X5280/X5283/X5284/X5285/ |
| | | X5286/X5287/X5288/X5290/X568/X6445/X6447/X6448/X6449/X6450/ |
| | | X6452/X6453/X6454/X6455/X6456/X6457/X6458/X6459/X6463/X6465/X6466/ |
| | | X6467/X6468/X6469/X6471/X6475/X6476/X6477/X6479/X6480/X6481/X6482/ |
| | | X6483/X6484/X7591/X7593/X7594/X7595/X7596/X7597/X7598/X7599/ |
| | | X7600/X7602/X7603/X7604/X7605/X7606/X7607/X7608/X7609/X7610/X7611/ |
| | | X7616/X7617/X7619/X7623/X7625/X7626/X7627/X7628/X7629/X7635/X7636/ |
| | | X7637/X7638/X7640/X7643/X8676/X8678/X8680/X8681/X8682/X8683/ |
| | | X8684/X8685/X8686/X8687/X8688/X8689/X8692/X8693/X8694/X8695/X8696/ |
| | | X8697/X8698/X8699/X8700/X8701/X8708/X8710/X8711/X8713/X8717/X8720/ |
| | | X8728/X8729/X8730/X8731/X907/X9639/X9640/X9641/X9643/X9644/X9645/ |
| | | X9646/X9647/X9648/X9649/X965/X9650/X9651/X9652/X9658/X9659/ |
| | | X966/X9660/X9661/X9662/X9663/X9673/X9675/X9676/X9678/X9690 |
| ASN_seq_aaDown_M | −Inf | X1219/X1222/X1223/X1224/X1225/X1226/X1228/X1229/X1230/X1231/X1232/ |
| | | X1233/X1234/X1235/X1236/X1237/X1238/X1239/X1240/X1431/X1435/X1898/ |
| | | X1899/X1901/X1902/X1903/X1904/X1905/X1906/X1907/X1908/X1910/ |
| | | X1911/X1912/X1914/X1916/X1917/X1918/X1919/X1920/X1921/X1922/X1923/ |
| | | X1924/X1925/X2035/X2146/X2148/X215/X2151/X2154/X216/X217/X218/ |
| | | X26/X2718/X2770/X2771/X2773/X2774/X2775/X2777/X2779/X2780/X2781/ |
| | | X2782/X2783/X2785/X2786/X2789/X2793/X2794/X2795/X2796/X2797/X2798/ |
| | | X2799/X2922/X2923/X2927/X3036/X3038/X3042/X3050/X32/X3755/X3768/ |
| | | X3769/X3816/X3819/X3820/X3823/X3825/X3826/X3827/X3829/X3833/X3834/ |
| | | X3835/X3836/X3966/X3970/X3972/X3976/X4074/X4079/X4087/X4090/ |
| | | X424/X425/X426/X427/X428/X429/X430/X4923/X4924/X4934/X4935/X4942/ |
| | | X4947/X4948/X4982/X4984/X4985/X4988/X5120/X5125/X5127/X5134/X5211/ |
| | | X5237/X532/X6164/X6165/X6170/X6171/X6176/X6181/X6182/X6186/X6187/ |
| | | X6325/X6334/X6400/X7/X7380/X7384/X7385/X7389/X7390/X7396/X7397/ |
| | | X7403/X7404/X744/X746/X747/X748/X749/X750/X751/X752/X753/X754/ |
| | | X755/X756/X8542/X8547/X8552/X8553/X8555/X8556/X901/X92/X93/X94/ |
| | | X9569/X9570 |
| ASN_seq_aaDown_P | −Inf | X1188/X1189/X1190/X1191/X1192/X1193/X1194/X170/X1865/X1866/X1867/ |
| | | X1868/X1869/X1894/X193/X194/X195/X196/X23/X2741/X2742/X2743/X2763/ |
| | | X3802/X3935/X397/X398/X399/X400/X401/X402/X5098/X713/X714/X715/ |
| | | X716/X717/X718/X719/X75/X76 |
| ASN_seq_aaDown_Q | −Inf | X10459/X117/X1219/X1220/X1221/X1222/X1223/X1224/X1225/X1226/X1227/ |
| | | X1228/X1229/X1230/X1231/X1232/X1233/X1234/X1235/X1236/X1237/X1238/ |
| | | X1239/X1240/X13/X1354/X1355/X1367/X1371/X1431/X1432/X1433/X1435/ |
| | | X1437/X1464/X1479/X1895/X1896/X1897/X1898/X1899/X1900/X1901/ |
| | | X1902/X1903/X1904/X1905/X1906/X1907/X1908/X1909/X1910/X1911/X1912/ |
| | | X1913/X1914/X1915/X1916/X1917/X1918/X1919/X1920/X1921/X1922/X1923/ |
| | | X1924/X1925/X2035/X2036/X2054/X2073/X2145/X2146/X2148/X2149/ |
| | | X215/X2150/X2151/X2152/X2154/X2157/X216/X2160/X217/X218/X2186/X2212/ |
| | | X249/X26/X263/X2764/X2765/X2766/X2767/X2768/X2769/X2770/X2771/ |
| | | X2772/X2773/X2774/X2775/X2776/X2777/X2778/X2779/X2780/X2781/X2782/ |
| | | X2783/X2784/X2785/X2786/X2788/X2789/X2790/X2791/X2792/X2793/ |
| | | X2794/X2795/X2796/X2797/X2798/X2799/X2922/X2923/X2924/X2925/X2926/ |
| | | X2927/X2928/X3032/X3033/X3034/X3035/X3036/X3038/X3039/X3040/X3042/ |
| | | X3044/X3045/X3046/X3050/X3051/X3052/X3053/X3056/X3059/X3060/ |
| | | X3061/X3062/X3116/X3117/X32/X3803/X3804/X3805/X3806/X3807/X3808/ |
| | | X3811/X3813/X3814/X3815/X3816/X3817/X3818/X3819/X3820/X3822/X3823/ |
| | | X3824/X3825/X3826/X3827/X3828/X3829/X3830/X3831/X3832/X3833/X3834/ |
| | | X3835/X3836/X3966/X3967/X3968/X3969/X3970/X3972/X3973/X3974/ |
| | | X3975/X3976/X3977/X3978/X4066/X4067/X4069/X4070/X4071/X4072/X4073/ |
| | | X4074/X4075/X4076/X4078/X4079/X4080/X4081/X4082/X4083/X4087/X4088/ |
| | | X4089/X4090/X4093/X4095/X4097/X4098/X4099/X4102/X4103/X4104/ |
| | | X4105/X4108/X4109/X4111/X4112/X4113/X424/X425/X426/X427/X428/X429/ |
| | | X430/X45/X4971/X4974/X4975/X4976/X4978/X4979/X4980/X4981/X4982/ |
| | | X4983/X4984/X4985/X4987/X4988/X4989/X4990/X4991/X508/X509/X5119/ |
| | | X5120/X5123/X5124/X5125/X5127/X5128/X5129/X513/X5130/X5131/X5132/ |
| | | X5133/X5134/X5135/X5200/X5201/X5202/X5203/X5204/X5205/X5206/X5207/ |
| | | X5208/X5210/X5211/X5212/X5213/X5214/X5215/X5217/X5220/X5221/ |
| | | X5222/X5225/X5227/X5229/X5230/X5231/X5232/X5233/X5237/X5238/X5239/ |
| | | X5240/X5242/X5243/X5244/X5245/X5248/X5249/X5252/X5253/X5254/X5255/ |
| | | X5256/X5257/X5258/X5259/X5260/X532/X6201/X6202/X6205/X6207/X6208/ |
| | | X6210/X6211/X6325/X6328/X6329/X6330/X6331/X6332/X6334/X6335/ |
| | | X6336/X6337/X6338/X6339/X6381/X6382/X6383/X6384/X6385/X6387/X6389/ |
| | | X6391/X6394/X6395/X6396/X6397/X6399/X6400/X6401/X6403/X6406/X6407/ |
| | | X6408/X6409/X6413/X6414/X6415/X6416/X6418/X6419/X6420/X6422/ |
| | | X6424/X6426/X6427/X6428/X6430/X6431/X6432/X6433/X6435/X6436/X6437/ |
| | | X6438/X6439/X6440/X6442/X6443/X6503/X7/X7412/X744/X745/X746/X747/ |
| | | X748/X749/X750/X7507/X751/X7510/X7511/X7513/X7514/X7515/X7516/ |
| | | X752/X753/X7537/X7538/X754/X7540/X7541/X7544/X7547/X755/X7550/ |
| | | X7551/X7553/X7556/X7557/X7558/X756/X7560/X7563/X7564/X7568/X7570/ |
| | | X7571/X7572/X7573/X7575/X7576/X7577/X7578/X7579/X7580/X7581/X7582/ |
| | | X7584/X7585/X7586/X7587/X7589/X7662/X851/X852/X853/X860/X863/ |
| | | X8631/X8632/X8645/X8647/X8654/X8655/X8657/X8660/X8661/X8664/X8665/ |
| | | X8666/X8667/X8668/X8669/X8670/X8673/X8674/X901/X903/X9031/X92/ |
| | | X93/X94/X9624/X9630/X9631/X9632/X9634/X9635 |
| ASN_seq_aaDown_R | −Inf | X10459/X117/X1220/X1221/X1222/X1223/X1225/X1227/X1228/X1229/X1230/ |
| | | X1231/X1232/X1233/X1234/X1235/X1236/X1237/X1238/X1239/X13/X1354/ |
| | | X1355/X1356/X1357/X1358/X1359/X1360/X1361/X1362/X1363/X1364/X1365/ |
| | | X1431/X1432/X1433/X1435/X1437/X1464/X1479/X1895/X1896/X1897/ |
| | | X1898/X1900/X1901/X1902/X1903/X1904/X1905/X1906/X1907/X1909/X1910/ |
| | | X1911/X1912/X1913/X1915/X1916/X1917/X1918/X1920/X1921/X1922/X1923/ |
| | | X1924/X1925/X2035/X2036/X2054/X2055/X2056/X2057/X2058/X2059/ |
| | | X2060/X2061/X2062/X2063/X2064/X2065/X2066/X2067/X2068/X2069/X214/ |
| | | X2145/X2146/X2148/X2149/X215/X2150/X2151/X2152/X2154/X2157/X2160/ |
| | | X217/X218/X2186/X2211/X2212/X263/X265/X2764/X2765/X2766/X2767/ |
| | | X2768/X2769/X2770/X2772/X2773/X2774/X2775/X2776/X2778/X2779/X2780/ |
| | | X2781/X2782/X2783/X2784/X2785/X2786/X2788/X2789/X2790/X2791/X2792/ |
| | | X2793/X2794/X2795/X2796/X2797/X2798/X29/X2922/X2923/X2924/X2925/ |
| | | X2926/X2927/X2928/X2944/X2945/X2946/X2947/X2948/X2949/X2950/ |
| | | X2951/X2952/X2953/X2954/X2955/X2956/X2957/X2958/X2959/X2960/X2961/ |
| | | X2962/X3032/X3033/X3034/X3035/X3036/X3038/X3039/X3040/X3042/X3044/ |
| | | X3045/X3046/X3050/X3051/X3052/X3053/X3056/X3059/X3060/X3061/ |
| | | X3062/X3072/X3115/X3116/X3117/X32/X3803/X3804/X3805/X3806/X3807/ |
| | | X3808/X3811/X3813/X3814/X3815/X3816/X3817/X3818/X3819/X3820/X3822/ |
| | | X3823/X3824/X3825/X3826/X3827/X3828/X3830/X3831/X3832/X3834/X3835/ |
| | | X3836/X3966/X3967/X3968/X3969/X3970/X3972/X3973/X3974/X3975/ |
| | | X3976/X3977/X3978/X3991/X3992/X3993/X3994/X3995/X3996/X3997/X3998/ |
| | | X3999/X4000/X4001/X4002/X4003/X4004/X4005/X4006/X4007/X4008/X4009/ |
| | | X4066/X4067/X4069/X4070/X4071/X4072/X4073/X4074/X4075/X4076/ |
| | | X4078/X4079/X4080/X4081/X4082/X4083/X4087/X4088/X4089/X4090/X4093/ |
| | | X4095/X4097/X4098/X4099/X4102/X4103/X4104/X4105/X4108/X4109/X4111/ |
| | | X4112/X4113/X4123/X4132/X4166/X425/X426/X428/X429/X430/X45/X4971/ |
| | | X4974/X4975/X4976/X4978/X4979/X4980/X4981/X4983/X4984/X4985/ |
| | | X4987/X4988/X4989/X4990/X4991/X508/X509/X510/X511/X5119/X512/X5120/ |
| | | X5123/X5124/X5125/X5127/X5128/X5129/X5130/X5131/X5132/X5133/ |
| | | X5134/X5135/X5141/X5142/X5143/X5144/X5145/X5146/X5147/X5148/X5149/ |
| | | X5150/X5151/X5152/X5153/X5154/X5155/X5200/X5201/X5202/X5203/X5204/ |
| | | X5205/X5206/X5207/X5208/X5210/X5211/X5212/X5213/X5214/X5215/ |
| | | X5217/X5220/X5221/X5222/X5225/X5227/X5229/X5230/X5231/X5232/X5233/ |
| | | X5237/X5238/X5239/X5240/X5242/X5243/X5244/X5245/X5248/X5249/X5252/ |
| | | X5253/X5254/X5255/X5256/X5257/X5258/X5259/X5260/X5278/X5282/ |
| | | X5289/X532/X6201/X6202/X6205/X6207/X6208/X6210/X6211/X6325/X6328/ |
| | | X6329/X6330/X6331/X6332/X6334/X6335/X6336/X6337/X6338/X6339/X6344/ |
| | | X6345/X6346/X6347/X6348/X6349/X6350/X6351/X6352/X6381/X6382/X6383/ |
| | | X6384/X6385/X6387/X6389/X6391/X6394/X6395/X6396/X6397/X6399/ |
| | | X6400/X6401/X6403/X6406/X6407/X6408/X6409/X6413/X6414/X6415/X6416/ |
| | | X6418/X6419/X6420/X6422/X6424/X6426/X6427/X6428/X6430/X6431/X6432/ |
| | | X6433/X6435/X6436/X6437/X6438/X6439/X6440/X6442/X6443/X6461/ |
| | | X6470/X6478/X6503/X7412/X745/X746/X747/X748/X750/X7507/X751/X7510/ |
| | | X7511/X7513/X7514/X7515/X7516/X7517/X7518/X7519/X7520/X753/X7537/ |
| | | X7538/X754/X7540/X7541/X7544/X7547/X755/X7550/X7551/X7553/X7556/ |
| | | X7557/X7558/X756/X7560/X7563/X7564/X7568/X7570/X7571/X7572/X7573/ |
| | | X7575/X7576/X7577/X7578/X7579/X7580/X7581/X7582/X7584/X7585/ |
| | | X7586/X7587/X7589/X7620/X7639/X7662/X84/X851/X852/X853/X854/X855/ |
| | | X856/X857/X858/X859/X8631/X8632/X8645/X8647/X8654/X8655/X8657/ |
| | | X8660/X8661/X8664/X8665/X8666/X8667/X8668/X8669/X8670/X8673/X8674/ |
| | | X8714/X8716/X90/X901/X903/X9031/X93/X94/X9624/X9630/X9631/X9632/ |
| | | X9634/X9635 |
| ASN_seq_aaDown_T | −Inf | X1333/X1334/X1335/X1336/X1337/X2028/X2029/X2031/X2032/X2033/X2034/ |
| | | X210/X256/X2916/X2918/X2919/X2920/X2921/X31/X356/X3962/X3964/X3965/ |
| | | X496/X497/X5118/X835/X836/X837/X89/X9/X91 |
| ASN_seq_aaDown_W | −Inf | X104/X110/X1219/X1223/X1224/X1225/X1226/X1228/X1231/X1232/X1233/ |
| | | X1237/X1240/X1329/X1898/X1899/X1901/X1904/X1905/X1907/X1908/X1910/ |
| | | X1914/X1916/X1919/X1920/X1923/X2035/X216/X217/X234/X235/X255/X26/ |
| | | X2770/X2771/X2773/X2777/X2779/X2782/X2783/X2785/X2793/X2796/X2799/ |
| | | X2922/X2923/X3816/X3819/X3825/X3829/X3833/X3834/X39/X3966/X3970/ |
| | | X424/X426/X427/X428/X452/X453/X491/X492/X4982/X4984/X5120/X7/ |
| | | X744/X746/X748/X749/X751/X752/X754/X799/X830/X831/X92 |
| ASN_seq_aaDown_Y | −Inf | X117/X1222/X1223/X1225/X1228/X1229/X1230/X1231/X1232/X1233/X1234/ |
| | | X1235/X1236/X1237/X1238/X1239/X13/X1354/X1431/X1432/X1433/X1435/ |
| | | X1437/X1898/X1901/X1902/X1903/X1904/X1905/X1906/X1907/X1910/X1911/ |
| | | X1912/X1913/X1915/X1916/X1917/X1918/X1920/X1921/X1922/X1923/X1924/ |
| | | X1925/X2035/X2145/X2146/X2148/X2149/X215/X2150/X2151/X2152/ |
| | | X2154/X2157/X217/X218/X263/X2768/X2769/X2770/X2773/X2774/X2775/X2776/ |
| | | X2779/X2780/X2781/X2782/X2783/X2785/X2786/X2789/X2790/X2791/ |
| | | X2792/X2793/X2794/X2795/X2796/X2797/X2798/X2922/X2923/X2927/X3033/ |
| | | X3034/X3035/X3036/X3038/X3039/X3042/X3044/X3046/X3050/X3051/X3052/ |
| | | X3053/X3059/X3060/X32/X3805/X3813/X3814/X3815/X3816/X3817/X3819/ |
| | | X3820/X3823/X3825/X3826/X3827/X3828/X3830/X3831/X3832/X3834/ |
| | | X3835/X3836/X3966/X3970/X3972/X3974/X3976/X4066/X4067/X4070/X4072/ |
| | | X4073/X4074/X4079/X4080/X4083/X4087/X4088/X4089/X4090/X4095/X4097/ |
| | | X4098/X4099/X4102/X4103/X4105/X4109/X425/X426/X428/X429/X430/ |
| | | X45/X4976/X4978/X4979/X4980/X4984/X4985/X4988/X4989/X4990/X4991/ |
| | | X508/X509/X5120/X5123/X5125/X5127/X5129/X5134/X5200/X5201/X5211/ |
| | | X5212/X5213/X5220/X5221/X5227/X5229/X5230/X5231/X5233/X5237/X5238/ |
| | | X5239/X5242/X5249/X5252/X5253/X532/X6207/X6208/X6211/X6325/X6328/ |
| | | X6334/X6336/X6384/X6394/X6395/X6400/X6401/X6406/X6418/X6424/ |
| | | X6426/X6427/X6428/X6433/X746/X747/X748/X750/X751/X7510/X753/X754/ |
| | | X755/X7556/X756/X7563/X7564/X7575/X851/X852/X8660/X901/X903/X93/X94 |
| ASN_seq_aaUp_A | −Inf | X115/X1195/X1198/X12/X1294/X1295/X1296/X1316/X1317/X1318/X1320/X1321/ |
| | | X1322/X1350/X1351/X1352/X1527/X1528/X1529/X1870/X1873/X1875/ |
| | | X1961/X1993/X1995/X1996/X2011/X2012/X2013/X2015/X2016/X2017/X2018/ |
| | | X2021/X2022/X2023/X2024/X2050/X2051/X2283/X2285/X2287/X245/X253/ |
| | | X260/X27/X2745/X2747/X2761/X2855/X2885/X2887/X2895/X2896/X2899/ |
| | | X2900/X2901/X2902/X2904/X2906/X2908/X2910/X2912/X2913/X2914/X2941/ |
| | | X3226/X3244/X3790/X3799/X3801/X3907/X3909/X3938/X3940/X3946/ |
| | | X3949/X3951/X3953/X3954/X3955/X3956/X3958/X3959/X3960/X4295/X4315/ |
| | | X44/X470/X485/X487/X4966/X4967/X504/X5073/X5103/X5106/X5107/X5108/ |
| | | X5109/X5111/X5113/X5115/X5499/X568/X6314/X6320/X6322/X6693/X7073/ |
| | | X720/X80/X801/X802/X822/X823/X824/X846/X847/X965/X966 |
| ASN_seq_aaUp_D | −Inf | X10494/X1356/X1359/X2055/X2057/X2063/X2120/X2944/X2946/X2947/X2951/ |
| | | X2953/X2957/X2996/X2997/X3002/X3004/X3992/X3993/X3995/X3996/X3998/ |
| | | X3999/X4002/X4006/X4025/X4026/X4032/X4033/X4037/X4038/X4039/ |
| | | X4040/X4042/X4130/X5141/X5142/X5144/X5145/X5147/X5149/X5151/X5154/ |
| | | X5161/X5164/X5165/X5169/X5170/X5171/X5172/X5173/X5174/X5178/X5179/ |
| | | X5180/X5181/X5276/X5287/X5293/X6344/X6345/X6346/X6348/X6349/ |
| | | X6352/X6353/X6355/X6356/X6357/X6358/X6359/X6363/X6364/X6365/X6366/ |
| | | X6367/X6368/X6369/X6462/X6468/X6476/X6483/X6490/X6491/X7517/X7519/ |
| | | X7520/X7521/X7522/X7524/X7525/X7526/X7527/X7528/X7529/X7530/ |
| | | X7615/X7617/X7618/X7621/X7628/X7637/X7651/X7652/X855/X8633/X8634/ |
| | | X8635/X8636/X8707/X8709/X8711/X8712/X8715/X8716/X8730/X8742/X9672/ |
| | | X9674/X9676/X9677/X9689 |
| ASN_seq_aaUp_E | −Inf | X10454/X10457/X10458/X10459/X10460/X10461/X10656/X10670/X10679/X10695/ |
| | | X10700/X10702/X11249/X11422/X1220/X1221/X1222/X1223/X1225/ |
| | | X1227/X1228/X1229/X1230/X1231/X1232/X1233/X1234/X1235/X1236/X1237/ |
| | | X1238/X1239/X1431/X1432/X1433/X1435/X1437/X1557/X1895/X1896/X1897/ |
| | | X1898/X1900/X1901/X1902/X1903/X1904/X1905/X1906/X1907/X1909/ |
| | | X1910/X1911/X1912/X1913/X1915/X1916/X1917/X1918/X1920/X1921/X1922/ |
| | | X1923/X1924/X1925/X2035/X2036/X2145/X2146/X2148/X2149/X215/X2150/ |
| | | X2151/X2152/X2154/X2157/X2160/X217/X218/X2331/X2332/X2764/X2765/ |
| | | X2766/X2767/X2768/X2769/X2770/X2772/X2773/X2774/X2775/X2776/X2778/ |
| | | X2779/X2780/X2781/X2782/X2783/X2784/X2785/X2786/X2787/X2788/ |
| | | X2789/X2790/X2791/X2792/X2793/X2794/X2795/X2796/X2797/X2798/X2922/ |
| | | X2923/X2924/X2925/X2926/X2927/X2928/X3032/X3033/X3034/X3035/X3036/ |
| | | X3038/X3039/X3040/X3042/X3044/X3045/X3046/X3050/X3051/X3052/ |
| | | X3053/X3056/X3059/X3060/X3061/X3062/X32/X3285/X3286/X3287/X3293/ |
| | | X3294/X3299/X3332/X3803/X3804/X3805/X3806/X3807/X3808/X3809/X3810/ |
| | | X3811/X3812/X3813/X3814/X3815/X3816/X3817/X3818/X3819/X3820/X3821/ |
| | | X3822/X3823/X3824/X3825/X3826/X3827/X3828/X3830/X3831/X3832/ |
| | | X3834/X3835/X3836/X3966/X3967/X3968/X3969/X3970/X3972/X3973/X3974/ |
| | | X3975/X3976/X3977/X3978/X4066/X4067/X4069/X4070/X4071/X4072/X4073/ |
| | | X4074/X4075/X4076/X4078/X4079/X4080/X4081/X4082/X4083/X4087/ |
| | | X4088/X4089/X4090/X4093/X4095/X4096/X4097/X4098/X4099/X4102/X4103/ |
| | | X4104/X4105/X4108/X4109/X4110/X4111/X4112/X4113/X4158/X425/X426/ |
| | | X428/X429/X430/X4373/X4374/X4376/X4378/X4380/X4384/X4385/X4389/ |
| | | X4391/X4392/X4394/X4397/X4442/X4968/X4969/X4970/X4971/X4972/X4973/ |
| | | X4974/X4975/X4976/X4977/X4978/X4979/X4980/X4981/X4983/X4984/ |
| | | X4985/X4986/X4987/X4988/X4989/X4990/X4991/X5119/X5120/X5123/X5124/ |
| | | X5125/X5127/X5128/X5129/X5130/X5131/X5132/X5133/X5134/X5135/X5200/ |
| | | X5201/X5202/X5203/X5204/X5205/X5206/X5207/X5208/X5210/X5211/ |
| | | X5212/X5213/X5214/X5215/X5217/X5218/X5219/X5220/X5221/X5222/X5225/ |
| | | X5227/X5228/X5229/X5230/X5231/X5232/X5233/X5237/X5238/X5239/X5240/ |
| | | X5241/X5242/X5243/X5244/X5245/X5248/X5249/X5250/X5252/X5253/ |
| | | X5254/X5255/X5256/X5257/X5258/X5259/X5260/X5309/X5310/X5315/X532/ |
| | | X5564/X5565/X5566/X5567/X5568/X5570/X5572/X5575/X5577/X5579/X5580/ |
| | | X5583/X5585/X5586/X5588/X5590/X5591/X5594/X5596/X5598/X5604/X5659/ |
| | | X6199/X6200/X6201/X6202/X6203/X6204/X6205/X6206/X6207/X6208/ |
| | | X6209/X6210/X6211/X6325/X6328/X6329/X6330/X6331/X6332/X6334/X6335/ |
| | | X6336/X6337/X6338/X6339/X6381/X6382/X6383/X6384/X6385/X6386/X6387/ |
| | | X6388/X6389/X6391/X6392/X6393/X6394/X6395/X6396/X6397/X6399/ |
| | | X6400/X6401/X6402/X6403/X6404/X6405/X6406/X6407/X6408/X6409/X6413/ |
| | | X6414/X6415/X6416/X6417/X6418/X6419/X6420/X6422/X6424/X6425/X6426/ |
| | | X6427/X6428/X6430/X6431/X6432/X6433/X6434/X6435/X6436/X6437/ |
| | | X6438/X6439/X6440/X6441/X6442/X6443/X6503/X6506/X6507/X6510/X6760/ |
| | | X6761/X6762/X6763/X6764/X6765/X6767/X6769/X6771/X6772/X6773/X6775/ |
| | | X6777/X6780/X6782/X6784/X6787/X6789/X6791/X6793/X6795/X6796/ |
| | | X6798/X6805/X6807/X6812/X7408/X7409/X7411/X7412/X745/X746/X747/ |
| | | X748/X750/X7507/X751/X7510/X7511/X7513/X7514/X7515/X7516/X753/X7537/ |
| | | X7538/X7539/X754/X7540/X7541/X7542/X7543/X7544/X7545/X7546/X7547/ |
| | | X7549/X755/X7550/X7551/X7552/X7553/X7554/X7555/X7556/X7557/ |
| | | X7558/X756/X7560/X7561/X7562/X7563/X7564/X7566/X7568/X7569/X7570/ |
| | | X7571/X7572/X7573/X7574/X7575/X7576/X7577/X7578/X7579/X7580/X7581/ |
| | | X7582/X7583/X7584/X7585/X7586/X7587/X7588/X7589/X7662/X7664/X7665/ |
| | | X7911/X7912/X7913/X7914/X7915/X7916/X7918/X7920/X7922/X7925/ |
| | | X7926/X7928/X7930/X7931/X7936/X7942/X7945/X7946/X7948/X7955/X7957/ |
| | | X7961/X7963/X7965/X7967/X7972/X7974/X7978/X7992/X8631/X8632/X8640/ |
| | | X8641/X8642/X8643/X8644/X8645/X8646/X8647/X8648/X8649/X8651/ |
| | | X8652/X8653/X8654/X8655/X8656/X8657/X8658/X8659/X8660/X8661/X8662/ |
| | | X8663/X8664/X8665/X8666/X8667/X8668/X8669/X8670/X8671/X8672/X8673/ |
| | | X8674/X8675/X8975/X8976/X8977/X8979/X8980/X8985/X8987/X8989/ |
| | | X8990/X8995/X9000/X9003/X9008/X901/X9014/X9016/X9018/X9020/X9025/ |
| | | X9027/X903/X9031/X9036/X9038/X9041/X9043/X9046/X93/X94/X9619/X9620/ |
| | | X9621/X9622/X9623/X9624/X9625/X9627/X9628/X9629/X9630/X9631/ |
| | | X9632/X9633/X9634/X9635/X9636/X9637/X9897/X9898/X9903/X9909/X9912/ |
| | | X9917/X9926/X9932/X9937/X9939/X9943/X9948/X9950/X9967/X9969/X9972 |
| ASN_seq_aaUp_F | −Inf | X10469/X10472/X10473/X10483/X10495/X11117/X1439/X2161/X2163/X3064/ |
| | | X3066/X3074/X4116/X4117/X4125/X4129/X4133/X4134/X5262/X5265/X5267/ |
| | | X5268/X5270/X5275/X5279/X5280/X5284/X5285/X5286/X5287/X5290/ |
| | | X6445/X6447/X6448/X6450/X6453/X6456/X6457/X6459/X6465/X6466/X6467/ |
| | | X6468/X6471/X6475/X6479/X6480/X6481/X6482/X6483/X7594/X7597/X7598/ |
| | | X7600/X7602/X7603/X7605/X7606/X7609/X7610/X7616/X7623/X7625/ |
| | | X7626/X7627/X7628/X7635/X7636/X7637/X7640/X7643/X8680/X8681/X8683/ |
| | | X8684/X8687/X8688/X8692/X8695/X8696/X8698/X8699/X8708/X8710/X8711/ |
| | | X8717/X8720/X8728/X8729/X8730/X9643/X9646/X9647/X9649/X9650/ |
| | | X9658/X9661/X9662/X9673/X9675/X9676/X9690 |
| ASN_seq_aaUp_G | −Inf | X10462/X10463/X10465/X10469/X10470/X10471/X10472/X10473/X10474/X10483/ |
| | | X10484/X10485/X10494/X10495/X11108/X11112/X11117/X11118/X11119/ |
| | | X11585/X1356/X1359/X1439/X2055/X2057/X2063/X2120/X2161/X2163/ |
| | | X2944/X2946/X2951/X2953/X2957/X2996/X2997/X3002/X3004/X3064/X3066/ |
| | | X3074/X3992/X3993/X3995/X3996/X3998/X4002/X4006/X4025/X4026/ |
| | | X4032/X4033/X4037/X4038/X4039/X4040/X4042/X4116/X4117/X4125/X4129/ |
| | | X4131/X4133/X4134/X5141/X5142/X5144/X5145/X5147/X5151/X5154/X5161/ |
| | | X5164/X5165/X5169/X5170/X5171/X5172/X5173/X5174/X5178/X5179/ |
| | | X5180/X5181/X5262/X5265/X5267/X5268/X5269/X5270/X5275/X5277/X5279/ |
| | | X5280/X5284/X5285/X5286/X5287/X5288/X5290/X5293/X6344/X6346/X6348/ |
| | | X6349/X6352/X6353/X6355/X6356/X6357/X6358/X6359/X6363/X6364/ |
| | | X6365/X6366/X6367/X6368/X6369/X6445/X6447/X6448/X6449/X6450/X6453/ |
| | | X6454/X6455/X6456/X6457/X6458/X6459/X6462/X6465/X6466/X6467/X6468/ |
| | | X6469/X6471/X6475/X6477/X6479/X6480/X6481/X6482/X6483/X6484/ |
| | | X6490/X6491/X7517/X7519/X7520/X7521/X7522/X7524/X7525/X7526/X7527/ |
| | | X7528/X7529/X7530/X7591/X7594/X7595/X7596/X7597/X7598/X7599/X7600/ |
| | | X7602/X7603/X7604/X7605/X7606/X7607/X7608/X7609/X7610/X7611/ |
| | | X7615/X7616/X7618/X7619/X7621/X7623/X7625/X7626/X7627/X7628/X7629/ |
| | | X7635/X7636/X7637/X7638/X7640/X7643/X7651/X7652/X855/X8633/X8634/ |
| | | X8635/X8636/X8676/X8678/X8680/X8681/X8682/X8683/X8684/X8685/X8686/ |
| | | X8687/X8688/X8689/X8692/X8693/X8694/X8695/X8696/X8697/X8698/ |
| | | X8699/X8700/X8701/X8707/X8708/X8709/X8710/X8711/X8712/X8713/X8715/ |
| | | X8716/X8717/X8720/X8728/X8729/X8730/X8731/X8742/X9639/X9640/X9641/ |
| | | X9643/X9644/X9645/X9646/X9647/X9648/X9649/X9650/X9651/X9652/ |
| | | X9658/X9659/X9660/X9661/X9662/X9663/X9672/X9673/X9674/X9675/X9676/ |
| | | X9677/X9678/X9689/X9690 |
| ASN_seq_aaUp_H | −Inf | X10469/X10472/X10473/X10483/X10494/X10495/X11117/X1356/X1357/X1359/ |
| | | X1360/X1439/X1440/X2055/X2057/X2058/X2059/X2061/X2063/X2120/X214/ |
| | | X2161/X2162/X2163/X2166/X254/X2944/X2945/X2946/X2947/X2949/X2951/ |
| | | X2953/X2954/X2957/X2996/X2997/X3002/X3004/X3064/X3065/X3066/ |
| | | X3070/X3073/X3074/X3991/X3992/X3993/X3995/X3996/X3998/X3999/X4002/ |
| | | X4004/X4006/X4025/X4026/X4032/X4033/X4037/X4038/X4039/X4040/X4042/ |
| | | X4114/X4116/X4117/X4119/X4124/X4125/X4127/X4129/X4130/X4133/ |
| | | X4134/X489/X510/X5141/X5142/X5144/X5145/X5146/X5147/X5149/X5151/ |
| | | X5154/X5161/X5164/X5165/X5169/X5170/X5171/X5172/X5173/X5174/X5178/ |
| | | X5179/X5180/X5181/X5262/X5264/X5265/X5267/X5268/X5270/X5273/X5275/ |
| | | X5276/X5279/X5280/X5283/X5284/X5285/X5286/X5287/X5290/X5293/ |
| | | X6344/X6345/X6346/X6348/X6349/X6352/X6353/X6355/X6356/X6357/X6358/ |
| | | X6359/X6363/X6364/X6365/X6366/X6367/X6368/X6369/X6445/X6447/X6448/ |
| | | X6450/X6452/X6453/X6456/X6457/X6459/X6462/X6463/X6465/X6466/ |
| | | X6467/X6468/X6471/X6475/X6476/X6479/X6480/X6481/X6482/X6483/X6490/ |
| | | X6491/X7517/X7519/X7520/X7521/X7522/X7524/X7525/X7526/X7527/X7528/ |
| | | X7529/X7530/X7593/X7594/X7597/X7598/X7600/X7602/X7603/X7605/ |
| | | X7606/X7609/X7610/X7615/X7616/X7617/X7618/X7621/X7623/X7625/X7626/ |
| | | X7627/X7628/X7635/X7636/X7637/X7640/X7643/X7651/X7652/X84/X855/ |
| | | X856/X8633/X8634/X8635/X8636/X8680/X8681/X8683/X8684/X8687/X8688/ |
| | | X8692/X8695/X8696/X8698/X8699/X8707/X8708/X8709/X8710/X8711/X8712/ |
| | | X8715/X8716/X8717/X8720/X8728/X8729/X8730/X8742/X907/X9643/ |
| | | X9646/X9647/X9649/X9650/X9658/X9661/X9662/X9672/X9673/X9674/X9675/ |
| | | X9676/X9677/X9689/X9690 |
| ASN_seq_aaUp_I | −Inf | X1356/X1357/X1359/X1360/X2055/X2057/X2058/X2059/X2061/X2063/X214/ |
| | | X2944/X2945/X2946/X2947/X2949/X2951/X2953/X2954/X2957/X3991/X3992/ |
| | | X3993/X3995/X3996/X3998/X3999/X4002/X4004/X4006/X510/X5141/X5142/ |
| | | X5144/X5145/X5146/X5147/X5149/X5151/X5154/X6344/X6345/X6346/ |
| | | X6348/X6349/X6352/X7517/X7519/X7520/X84/X855/X856/X8716 |
| ASN_seq_aaUp_K | −Inf | X1316/X1317/X1318/X1320/X1321/X1322/X2011/X2012/X2013/X2015/X2016/ |
| | | X2017/X2021/X2022/X2023/X253/X2895/X2899/X2900/X2901/X2904/X2910/ |
| | | X2913/X2914/X3946/X3953/X3954/X3955/X3958/X3959/X485/X487/X5106/ |
| | | X5107/X5108/X822/X823/X824 |
| ASN_seq_aaUp_L | −Inf | X356 |
| ASN_seq_aaUp_M | −Inf | X1189/X1194/X1356/X1359/X1866/X1867/X2055/X2057/X2063/X2742/X2743/ |
| | | X2944/X2946/X2951/X2953/X2957/X3935/X3992/X3993/X3995/X3996/X3998/ |
| | | X400/X4002/X4006/X5098/X5141/X5142/X5144/X5145/X5147/X5151/X5154/ |
| | | X6344/X6346/X6348/X6349/X6352/X718/X7517/X7519/X7520/X855/X8716 |
| ASN_seq_aaUp_P | −Inf | X10462/X10463/X10465/X10469/X10470/X10471/X10472/X10473/X10474/X10483/ |
| | | X10484/X10485/X10494/X10495/X11108/X11112/X11117/X11118/X11119/ |
| | | X11585/X1356/X1359/X1439/X1950/X2055/X2057/X2063/X2120/X2161/ |
| | | X2163/X2825/X2944/X2946/X2951/X2953/X2957/X2996/X2997/X3002/X3004/ |
| | | X3064/X3066/X3074/X3863/X3992/X3993/X3995/X3996/X3998/X4002/ |
| | | X4006/X4025/X4026/X4032/X4033/X4037/X4038/X4039/X4040/X4042/X4116/ |
| | | X4117/X4125/X4129/X4131/X4133/X4134/X5141/X5142/X5144/X5145/X5147/ |
| | | X5151/X5154/X5161/X5164/X5165/X5169/X5170/X5171/X5172/X5173/ |
| | | X5174/X5178/X5179/X5180/X5181/X5262/X5265/X5267/X5268/X5269/X5270/ |
| | | X5275/X5277/X5279/X5280/X5284/X5285/X5286/X5287/X5288/X5290/X5293/ |
| | | X6344/X6346/X6348/X6349/X6352/X6353/X6355/X6356/X6357/X6358/ |
| | | X6359/X6363/X6364/X6365/X6366/X6367/X6368/X6369/X6445/X6447/X6448/ |
| | | X6449/X6450/X6453/X6454/X6455/X6456/X6457/X6458/X6459/X6462/X6465/ |
| | | X6466/X6467/X6468/X6469/X6471/X6475/X6477/X6479/X6480/X6481/ |
| | | X6482/X6483/X6484/X6490/X6491/X7517/X7519/X7520/X7521/X7522/X7524/ |
| | | X7525/X7526/X7527/X7528/X7529/X7530/X7591/X7594/X7595/X7596/X7597/ |
| | | X7598/X7599/X7600/X7602/X7603/X7604/X7605/X7606/X7607/X7608/ |
| | | X7609/X7610/X7611/X7615/X7616/X7618/X7619/X7621/X7623/X7625/X7626/ |
| | | X7627/X7628/X7629/X7635/X7636/X7637/X7638/X7640/X7643/X7651/X7652/ |
| | | X855/X8633/X8634/X8635/X8636/X8676/X8678/X8680/X8681/X8682/X8683/ |
| | | X8684/X8685/X8686/X8687/X8688/X8689/X8692/X8693/X8694/X8695/ |
| | | X8696/X8697/X8698/X8699/X8700/X8701/X8707/X8708/X8709/X8710/X8711/ |
| | | X8712/X8713/X8715/X8716/X8717/X8720/X8728/X8729/X8730/X8731/X8742/ |
| | | X9639/X9640/X9641/X9643/X9644/X9645/X9646/X9647/X9648/X9649/ |
| | | X9650/X9651/X9652/X9658/X9659/X9660/X9661/X9662/X9663/X9672/X9673/ |
| | | X9674/X9675/X9676/X9677/X9678/X9689/X9690 |
| ASN_seq_aaUp_Q | −Inf | X1274/X1971/X2857 |
| ASN_seq_aaUp_R | −Inf | X1316/X1317/X1318/X1320/X1321/X1322/X2011/X2012/X2013/X2015/X2016/ |
| | | X2017/X2021/X2022/X2023/X253/X2895/X2899/X2900/X2901/X2904/X2910/ |
| | | X2913/X2914/X3946/X3953/X3954/X3955/X3958/X3959/X485/X487/X5106/ |
| | | X5107/X5108/X5287/X6468/X6483/X7628/X7637/X822/X823/X824/X8711/ |
| | | X8730/X9676 |
| ASN_seq_aaUp_S | −Inf | X170 |
| ASN_seq_aaUp_T | −Inf | X10462/X10463/X10465/X10469/X10470/X10471/X10472/X10473/X10474/X10483/ |
| | | X10484/X10485/X10494/X10495/X11108/X11112/X11117/X11118/X11119/ |
| | | X11585/X1356/X1357/X1359/X1360/X1439/X1440/X2055/X2057/X2058/ |
| | | X2059/X2061/X2063/X2120/X214/X2161/X2162/X2163/X2166/X254/X2944/ |
| | | X2945/X2946/X2947/X2949/X2951/X2953/X2954/X2957/X2996/X2997/X3002/ |
| | | X3004/X3064/X3065/X3066/X3070/X3073/X3074/X3991/X3992/X3993/ |
| | | X3995/X3996/X3998/X3999/X4002/X4004/X4006/X4025/X4026/X4032/X4033/ |
| | | X4037/X4038/X4039/X4040/X4042/X4114/X4116/X4117/X4119/X4124/X4125/ |
| | | X4127/X4129/X4130/X4131/X4133/X4134/X489/X510/X5141/X5142/X5144/ |
| | | X5145/X5146/X5147/X5149/X5151/X5154/X5161/X5164/X5165/X5169/ |
| | | X5170/X5171/X5172/X5173/X5174/X5178/X5179/X5180/X5181/X5262/X5264/ |
| | | X5265/X5267/X5268/X5269/X5270/X5273/X5275/X5276/X5277/X5279/X5280/ |
| | | X5283/X5284/X5285/X5286/X5287/X5288/X5290/X5293/X6344/X6345/ |
| | | X6346/X6348/X6349/X6352/X6353/X6355/X6356/X6357/X6358/X6359/X6363/ |
| | | X6364/X6365/X6366/X6367/X6368/X6369/X6445/X6447/X6448/X6449/X6450/ |
| | | X6452/X6453/X6454/X6455/X6456/X6457/X6458/X6459/X6462/X6463/ |
| | | X6465/X6466/X6467/X6468/X6469/X6471/X6475/X6476/X6477/X6479/X6480/ |
| | | X6481/X6482/X6483/X6484/X6490/X6491/X7517/X7519/X7520/X7521/X7522/ |
| | | X7524/X7525/X7526/X7527/X7528/X7529/X7530/X7591/X7593/X7594/ |
| | | X7595/X7596/X7597/X7598/X7599/X7600/X7602/X7603/X7604/X7605/X7606/ |
| | | X7607/X7608/X7609/X7610/X7611/X7615/X7616/X7617/X7618/X7619/X7621/ |
| | | X7623/X7625/X7626/X7627/X7628/X7629/X7635/X7636/X7637/X7638/ |
| | | X7640/X7643/X7651/X7652/X84/X855/X856/X8633/X8634/X8635/X8636/X8676/ |
| | | X8678/X8680/X8681/X8682/X8683/X8684/X8685/X8686/X8687/X8688/ |
| | | X8689/X8692/X8693/X8694/X8695/X8696/X8697/X8698/X8699/X8700/X8701/ |
| | | X8707/X8708/X8709/X8710/X8711/X8712/X8713/X8715/X8716/X8717/X8720/ |
| | | X8728/X8729/X8730/X8731/X8742/X907/X9639/X9640/X9641/X9643/X9644/ |
| | | X9645/X9646/X9647/X9648/X9649/X9650/X9651/X9652/X9658/X9659/ |
| | | X9660/X9661/X9662/X9663/X9672/X9673/X9674/X9675/X9676/X9677/X9678/ |
| | | X9689/X9690 |
| ASN_seq_{—} | −Inf | X1267/X1316/X1317/X1318/X1320/X1321/X1322/X1950/X1953/X2011/X2012/ |
| aaUp_V | | X2013/X2015/X2016/X2017/X2018/X2021/X2022/X2023/X253/X2825/X2828/ |
| | | X2895/X2896/X2899/X2900/X2901/X2902/X2904/X2910/X2913/X2914/X3863/ |
| | | X3865/X3940/X3946/X3953/X3954/X3955/X3956/X3958/X3959/X485/ |
| | | X487/X5103/X5106/X5107/X5108/X5109/X5318/X6314/X822/X823/X824 |
| ASN_seq_SS_sspro8C | −Inf | X1527/X1528/X1529/X170/X1880/X1881/X2283/X2285/X2748/X2753/X2754/ |
| | | X3207/X356/X3791/X3794/X3795/X4961/X5385/X5386/X568/X6895/X965/X966 |
| ASN_seq_SS_sspro8E | −Inf | X10459/X1220/X1221/X1222/X1223/X1225/X1227/X1228/X1229/X1230/X1231/ |
| | | X1232/X1233/X1234/X1235/X1236/X1237/X1238/X1239/X1240/X1267/X1317/ |
| | | X1320/X1321/X1322/X1356/X1358/X1359/X1361/X1365/X1431/X1432/ |
| | | X1433/X1435/X1437/X1895/X1896/X1897/X1898/X1900/X1901/X1902/X1903/ |
| | | X1904/X1905/X1906/X1907/X1909/X1910/X1911/X1912/X1913/X1914/X1915/ |
| | | X1916/X1917/X1918/X1919/X1920/X1921/X1922/X1923/X1924/X1925/ |
| | | X1953/X2012/X2021/X2022/X2023/X2035/X2036/X2055/X2056/X2057/X2062/ |
| | | X2063/X2064/X2066/X2067/X2069/X2145/X2146/X2148/X2149/X215/X2150/ |
| | | X2151/X2152/X2154/X2157/X2160/X217/X218/X253/X2764/X2765/X2766/ |
| | | X2767/X2768/X2769/X2770/X2772/X2773/X2774/X2775/X2776/X2777/X2778/ |
| | | X2779/X2780/X2781/X2782/X2783/X2784/X2785/X2786/X2788/X2789/ |
| | | X2790/X2791/X2792/X2793/X2794/X2795/X2796/X2797/X2798/X2799/X2828/ |
| | | X2910/X2913/X2914/X2922/X2923/X2924/X2925/X2926/X2927/X2928/X2944/ |
| | | X2946/X2947/X2950/X2951/X2952/X2953/X2955/X2956/X2957/X2958/ |
| | | X2959/X2960/X2962/X3032/X3033/X3034/X3035/X3036/X3038/X3039/X3040/ |
| | | X3042/X3044/X3045/X3046/X3050/X3051/X3052/X3053/X3056/X3059/X3060/ |
| | | X3061/X3062/X3072/X32/X3803/X3804/X3805/X3806/X3807/X3808/X3811/ |
| | | X3813/X3814/X3815/X3816/X3817/X3818/X3819/X3820/X3822/X3823/ |
| | | X3824/X3825/X3826/X3827/X3828/X3829/X3830/X3831/X3832/X3833/X3834/ |
| | | X3835/X3836/X3865/X3958/X3959/X3966/X3967/X3968/X3969/X3970/X3972/ |
| | | X3973/X3974/X3975/X3976/X3977/X3978/X3992/X3993/X3994/X3995/ |
| | | X3996/X3997/X3998/X3999/X4000/X4001/X4002/X4005/X4006/X4007/X4008/ |
| | | X4009/X4066/X4067/X4069/X4070/X4071/X4072/X4073/X4074/X4075/X4076/ |
| | | X4078/X4079/X4080/X4081/X4082/X4083/X4087/X4088/X4089/X4090/ |
| | | X4093/X4095/X4097/X4098/X4099/X4102/X4103/X4104/X4105/X4108/X4109/ |
| | | X4111/X4112/X4113/X4123/X4132/X425/X426/X428/X429/X430/X487/X4971/ |
| | | X4974/X4975/X4976/X4978/X4979/X4980/X4981/X4982/X4983/X4984/ |
| | | X4985/X4987/X4988/X4989/X4990/X4991/X511/X5119/X5120/X5123/X5124/ |
| | | X5125/X5127/X5128/X5129/X5130/X5131/X5132/X5133/X5134/X5135/X5141/ |
| | | X5142/X5143/X5144/X5145/X5147/X5148/X5149/X5150/X5151/X5152/ |
| | | X5153/X5154/X5155/X5200/X5201/X5202/X5203/X5204/X5205/X5206/X5207/ |
| | | X5208/X5210/X5211/X5212/X5213/X5214/X5215/X5217/X5220/X5221/X5222/ |
| | | X5225/X5227/X5229/X5230/X5231/X5232/X5233/X5237/X5238/X5239/ |
| | | X5240/X5242/X5243/X5244/X5245/X5248/X5249/X5252/X5253/X5254/X5255/ |
| | | X5256/X5257/X5258/X5259/X5260/X5278/X5282/X5289/X532/X6201/X6202/ |
| | | X6205/X6207/X6208/X6210/X6211/X6325/X6328/X6329/X6330/X6331/X6332/ |
| | | X6334/X6335/X6336/X6337/X6338/X6339/X6344/X6345/X6346/X6347/ |
| | | X6348/X6349/X6350/X6351/X6352/X6381/X6382/X6383/X6384/X6385/X6387/ |
| | | X6389/X6391/X6394/X6395/X6396/X6397/X6399/X6400/X6401/X6403/X6406/ |
| | | X6407/X6408/X6409/X6413/X6414/X6415/X6416/X6418/X6419/X6420/ |
| | | X6422/X6424/X6426/X6427/X6428/X6430/X6431/X6432/X6433/X6435/X6436/ |
| | | X6437/X6438/X6439/X6440/X6442/X6443/X6461/X6470/X6478/X6503/X7412/ |
| | | X745/X746/X747/X748/X750/X7507/X751/X7510/X7511/X7513/X7514/ |
| | | X7515/X7516/X7517/X7518/X7519/X7520/X753/X7537/X7538/X754/X7540/ |
| | | X7541/X7544/X7547/X755/X7550/X7551/X7553/X7556/X7557/X7558/X756/ |
| | | X7560/X7563/X7564/X7568/X7570/X7571/X7572/X7573/X7575/X7576/X7577/ |
| | | X7578/X7579/X7580/X7581/X7582/X7584/X7585/X7586/X7587/X7589/X7620/ |
| | | X7639/X7662/X823/X824/X854/X855/X858/X8631/X8632/X8645/X8647/ |
| | | X8654/X8655/X8657/X8660/X8661/X8664/X8665/X8666/X8667/X8668/X8669/ |
| | | X8670/X8673/X8674/X8714/X8716/X901/X903/X9031/X93/X94/X9624/ |
| | | X9630/X9631/X9632/X9634/X9635 |
| ASN_seq_SS_sspro8H | −Inf | X117/X1219/X1222/X1223/X1224/X1225/X1226/X1228/X1229/X1230/X1231/ |
| | | X1232/X1233/X1234/X1235/X1236/X1237/X1238/X1239/X1240/X1267/X13/ |
| | | X1354/X1431/X1435/X1898/X1899/X1901/X1902/X1903/X1904/X1905/X1906/ |
| | | X1907/X1908/X1910/X1911/X1912/X1914/X1915/X1916/X1917/X1918/X1919/ |
| | | X1920/X1921/X1922/X1923/X1924/X1925/X1953/X2035/X2146/X2148/ |
| | | X215/X2151/X2154/X2157/X216/X217/X218/X26/X263/X2769/X2770/X2771/ |
| | | X2773/X2774/X2775/X2777/X2779/X2780/X2781/X2782/X2783/X2785/X2786/ |
| | | X2789/X2790/X2791/X2792/X2793/X2794/X2795/X2796/X2797/X2798/ |
| | | X2799/X2828/X2922/X2923/X2927/X3036/X3038/X3039/X3042/X3046/X3050/ |
| | | X3053/X32/X3813/X3814/X3815/X3816/X3817/X3819/X3820/X3823/X3825/ |
| | | X3826/X3827/X3829/X3830/X3831/X3832/X3833/X3834/X3835/X3836/X3966/ |
| | | X3970/X3972/X3974/X3976/X4067/X4070/X4074/X4079/X4083/X4087/ |
| | | X4090/X4097/X4105/X424/X425/X426/X427/X428/X429/X430/X45/X4978/X4979/ |
| | | X4980/X4982/X4984/X4985/X4988/X4989/X4990/X4991/X508/X509/X5120/ |
| | | X5123/X5125/X5127/X5129/X5134/X5200/X5211/X5213/X5220/X5229/ |
| | | X5233/X5237/X5242/X5253/X532/X6207/X6208/X6211/X6325/X6328/X6334/ |
| | | X6336/X6384/X6394/X6400/X6406/X6418/X6426/X6433/X7/X744/X746/X747/ |
| | | X748/X749/X750/X751/X7510/X752/X753/X754/X755/X7556/X756/X7563/ |
| | | X7575/X851/X852/X8660/X901/X92/X93/X94 |
| ASN_seq_SS_sspro8S | −Inf | X10459/X10494/X1220/X1221/X1222/X1223/X1225/X1227/X1228/X1229/X1230/ |
| | | X1231/X1232/X1233/X1234/X1235/X1236/X1237/X1238/X1239/X1240/ |
| | | X1356/X1357/X1358/X1359/X1360/X1361/X1362/X1363/X1364/X1365/X1431/ |
| | | X1432/X1433/X1435/X1437/X1557/X1895/X1896/X1897/X1898/X1900/X1901/ |
| | | X1902/X1903/X1904/X1905/X1906/X1907/X1909/X1910/X1911/X1912/ |
| | | X1913/X1914/X1915/X1916/X1917/X1918/X1919/X1920/X1921/X1922/X1923/ |
| | | X1924/X1925/X2035/X2036/X2055/X2056/X2057/X2058/X2059/X2060/X2061/ |
| | | X2062/X2063/X2064/X2065/X2066/X2067/X2068/X2069/X2120/X214/X2145/ |
| | | X2146/X2148/X2149/X215/X2150/X2151/X2152/X2154/X2157/X2160/X217/ |
| | | X218/X2331/X2332/X265/X2764/X2765/X2766/X2767/X2768/X2769/X2770/ |
| | | X2772/X2773/X2774/X2775/X2776/X2777/X2778/X2779/X2780/X2781/ |
| | | X2782/X2783/X2784/X2785/X2786/X2787/X2788/X2789/X2790/X2791/X2792/ |
| | | X2793/X2794/X2795/X2796/X2797/X2798/X2799/X29/X2922/X2923/X2924/ |
| | | X2925/X2926/X2927/X2928/X2944/X2945/X2946/X2947/X2948/X2949/X2950/ |
| | | X2951/X2952/X2953/X2954/X2955/X2956/X2957/X2958/X2959/X2960/ |
| | | X2961/X2962/X2996/X2997/X3002/X3004/X3032/X3033/X3034/X3035/X3036/ |
| | | X3038/X3039/X3040/X3042/X3044/X3045/X3046/X3050/X3051/X3052/X3053/ |
| | | X3056/X3059/X3060/X3061/X3062/X3072/X32/X3285/X3286/X3287/X3293/ |
| | | X3803/X3804/X3805/X3806/X3807/X3808/X3810/X3811/X3812/X3813/ |
| | | X3814/X3815/X3816/X3817/X3818/X3819/X3820/X3821/X3822/X3823/X3824/ |
| | | X3825/X3826/X3827/X3828/X3829/X3830/X3831/X3832/X3833/X3834/X3835/ |
| | | X3836/X3966/X3967/X3968/X3969/X3970/X3972/X3973/X3974/X3975/ |
| | | X3976/X3977/X3978/X3991/X3992/X3993/X3994/X3995/X3996/X3997/X3998/ |
| | | X3999/X4000/X4001/X4002/X4003/X4004/X4005/X4006/X4007/X4008/X4009/ |
| | | X4025/X4026/X4032/X4033/X4037/X4038/X4039/X4040/X4042/X4066/ |
| | | X4067/X4069/X4070/X4071/X4072/X4073/X4074/X4075/X4076/X4078/X4079/ |
| | | X4080/X4081/X4082/X4083/X4087/X4088/X4089/X4090/X4093/X4095/X4096/ |
| | | X4097/X4098/X4099/X4102/X4103/X4104/X4105/X4108/X4109/X4111/ |
| | | X4112/X4113/X4123/X4132/X425/X426/X428/X429/X430/X4373/X4374/X4376/ |
| | | X4378/X4384/X4389/X4392/X4394/X4969/X4970/X4971/X4973/X4974/ |
| | | X4975/X4976/X4977/X4978/X4979/X4980/X4981/X4982/X4983/X4984/X4985/ |
| | | X4986/X4987/X4988/X4989/X4990/X4991/X510/X511/X5119/X512/X5120/ |
| | | X5123/X5124/X5125/X5127/X5128/X5129/X5130/X5131/X5132/X5133/X5134/ |
| | | X5135/X5141/X5142/X5143/X5144/X5145/X5146/X5147/X5148/X5149/X5150/ |
| | | X5151/X5152/X5153/X5154/X5155/X5161/X5164/X5165/X5169/X5170/ |
| | | X5171/X5172/X5173/X5174/X5178/X5179/X5180/X5181/X5200/X5201/X5202/ |
| | | X5203/X5204/X5205/X5206/X5207/X5208/X5210/X5211/X5212/X5213/X5214/ |
| | | X5215/X5217/X5218/X5219/X5220/X5221/X5222/X5225/X5227/X5228/ |
| | | X5229/X5230/X5231/X5232/X5233/X5237/X5238/X5239/X5240/X5241/X5242/ |
| | | X5243/X5244/X5245/X5248/X5249/X5252/X5253/X5254/X5255/X5256/X5257/ |
| | | X5258/X5259/X5260/X5278/X5282/X5289/X5293/X5309/X5310/X532/ |
| | | X5564/X5565/X5566/X5570/X5575/X5580/X5583/X5586/X5588/X5590/X5594/ |
| | | X6200/X6201/X6202/X6204/X6205/X6206/X6207/X6208/X6210/X6211/X6325/ |
| | | X6328/X6329/X6330/X6331/X6332/X6334/X6335/X6336/X6337/X6338/ |
| | | X6339/X6344/X6345/X6346/X6347/X6348/X6349/X6350/X6351/X6352/X6353/ |
| | | X6355/X6356/X6357/X6358/X6359/X6363/X6364/X6365/X6366/X6367/X6368/ |
| | | X6369/X6381/X6382/X6383/X6384/X6385/X6386/X6387/X6388/X6389/ |
| | | X6391/X6392/X6393/X6394/X6395/X6396/X6397/X6399/X6400/X6401/X6403/ |
| | | X6404/X6405/X6406/X6407/X6408/X6409/X6413/X6414/X6415/X6416/X6417/ |
| | | X6418/X6419/X6420/X6422/X6424/X6425/X6426/X6427/X6428/X6430/ |
| | | X6431/X6432/X6433/X6434/X6435/X6436/X6437/X6438/X6439/X6440/X6441/ |
| | | X6442/X6443/X6461/X6462/X6470/X6478/X6490/X6491/X6503/X6506/X6507/ |
| | | X6760/X6762/X6764/X6767/X6772/X6773/X6777/X6787/X6793/X6796/ |
| | | X6798/X6812/X7409/X7412/X745/X746/X747/X748/X750/X7507/X751/X7510/ |
| | | X7511/X7513/X7514/X7515/X7516/X7517/X7518/X7519/X7520/X7521/X7522/ |
| | | X7524/X7525/X7526/X7527/X7528/X7529/X753/X7530/X7537/X7538/X7539/ |
| | | X754/X7540/X7541/X7544/X7545/X7547/X7549/X755/X7550/X7551/X7553/ |
| | | X7554/X7555/X7556/X7557/X7558/X756/X7560/X7561/X7562/X7563/ |
| | | X7564/X7566/X7568/X7570/X7571/X7572/X7573/X7574/X7575/X7576/X7577/ |
| | | X7578/X7579/X7580/X7581/X7582/X7583/X7584/X7585/X7586/X7587/X7589/ |
| | | X7615/X7618/X7620/X7621/X7639/X7651/X7652/X7662/X7664/X7665/ |
| | | X7912/X7914/X7916/X7922/X7926/X7931/X7946/X7948/X7961/X84/X854/X855/ |
| | | X856/X857/X858/X859/X8631/X8632/X8633/X8634/X8635/X8636/X8642/ |
| | | X8645/X8646/X8647/X8648/X8651/X8654/X8655/X8657/X8658/X8659/X8660/ |
| | | X8661/X8664/X8665/X8666/X8667/X8668/X8669/X8670/X8673/X8674/ |
| | | X8707/X8709/X8712/X8714/X8715/X8716/X8742/X8977/X8979/X8990/X90/ |
| | | X9000/X901/X903/X9031/X9041/X9043/X93/X94/X9624/X9625/X9630/X9631/ |
| | | X9632/X9634/X9635/X9672/X9674/X9677/X9689/X9903/X9932 |
| ASN_seq_SS_sspro8T | −Inf | X10469/X10472/X10473/X10483/X10495/X11117/X1439/X1440/X2161/X2162/ |
| | | X2163/X2166/X254/X3064/X3065/X3066/X3070/X3073/X3074/X4114/X4116/ |
| | | X4117/X4119/X4124/X4125/X4127/X4129/X4130/X4133/X4134/X489/X5262/ |
| | | X5264/X5265/X5267/X5268/X5270/X5273/X5275/X5276/X5279/X5280/ |
| | | X5283/X5284/X5285/X5286/X5287/X5290/X6445/X6447/X6448/X6450/X6452/ |
| | | X6453/X6456/X6457/X6459/X6463/X6465/X6466/X6467/X6468/X6471/X6475/ |
| | | X6476/X6479/X6480/X6481/X6482/X6483/X7593/X7594/X7597/X7598/ |
| | | X7600/X7602/X7603/X7605/X7606/X7609/X7610/X7616/X7617/X7623/X7625/ |
| | | X7626/X7627/X7628/X7635/X7636/X7637/X7640/X7643/X8680/X8681/X8683/ |
| | | X8684/X8687/X8688/X8692/X8695/X8696/X8698/X8699/X8708/X8710/ |
| | | X8711/X8717/X8720/X8728/X8729/X8730/X907/X9643/X9646/X9647/X9649/ |
| | | X9650/X9658/X9661/X9662/X9673/X9675/X9676/X9690 |
| ASN_seq_SS_ssproE | −Inf | X10459/X105/X1220/X1221/X1222/X1223/X1225/X1227/X1228/X1229/X1230/ |
| | | X1231/X1232/X1233/X1234/X1235/X1236/X1237/X1238/X1239/X1240/X1267/ |
| | | X1283/X1284/X1317/X1320/X1321/X1356/X1358/X1359/X1361/X1365/ |
| | | X1431/X1432/X1433/X1435/X1437/X1895/X1896/X1897/X1898/X1900/X1901/ |
| | | X1902/X1903/X1904/X1905/X1906/X1907/X1909/X1910/X1911/X1912/X1913/ |
| | | X1914/X1915/X1916/X1917/X1918/X1919/X1920/X1921/X1922/X1923/ |
| | | X1924/X1925/X1953/X1980/X2012/X2021/X2023/X2035/X2036/X2055/X2056/ |
| | | X2057/X2062/X2063/X2064/X2066/X2067/X2069/X2145/X2146/X2148/X2149/ |
| | | X215/X2150/X2151/X2152/X2154/X2157/X2160/X217/X218/X241/X253/ |
| | | X2764/X2765/X2766/X2767/X2768/X2769/X2770/X2772/X2773/X2774/X2775/ |
| | | X2776/X2777/X2778/X2779/X2780/X2781/X2782/X2783/X2784/X2785/X2786/ |
| | | X2788/X2789/X2790/X2791/X2792/X2793/X2794/X2795/X2796/X2797/ |
| | | X2798/X2799/X2828/X2910/X2913/X2922/X2923/X2924/X2925/X2926/X2927/ |
| | | X2928/X2944/X2946/X2947/X2950/X2951/X2952/X2953/X2955/X2956/X2957/ |
| | | X2958/X2959/X2960/X2962/X3032/X3033/X3034/X3035/X3036/X3038/ |
| | | X3039/X3040/X3042/X3044/X3045/X3046/X3050/X3051/X3052/X3053/X3056/ |
| | | X3059/X3060/X3061/X3062/X3072/X32/X3803/X3804/X3805/X3806/X3807/ |
| | | X3808/X3811/X3813/X3814/X3815/X3816/X3817/X3818/X3819/X3820/X3822/ |
| | | X3823/X3824/X3825/X3826/X3827/X3828/X3829/X3830/X3831/X3832/ |
| | | X3833/X3834/X3835/X3836/X3865/X3958/X3966/X3967/X3968/X3969/X3970/ |
| | | X3972/X3973/X3974/X3975/X3976/X3977/X3978/X3992/X3993/X3994/X3995/ |
| | | X3996/X3997/X3998/X3999/X4000/X4001/X4002/X4005/X4006/X4007/ |
| | | X4008/X4009/X4066/X4067/X4069/X4070/X4071/X4072/X4073/X4074/X4075/ |
| | | X4076/X4078/X4079/X4080/X4081/X4082/X4083/X4087/X4088/X4089/X4090/ |
| | | X4093/X4095/X4097/X4098/X4099/X4102/X4103/X4104/X4105/X4108/ |
| | | X4109/X4111/X4112/X4113/X4123/X425/X426/X428/X429/X430/X461/X464/ |
| | | X487/X4971/X4974/X4975/X4976/X4978/X4979/X4980/X4981/X4982/X4983/ |
| | | X4984/X4985/X4987/X4988/X4989/X4990/X4991/X511/X5119/X5120/X5123/ |
| | | X5124/X5125/X5127/X5128/X5129/X5130/X5131/X5132/X5133/X5134/ |
| | | X5135/X5141/X5142/X5143/X5144/X5145/X5147/X5148/X5149/X5150/X5151/ |
| | | X5152/X5153/X5154/X5155/X5200/X5201/X5202/X5203/X5204/X5205/X5206/ |
| | | X5207/X5208/X5210/X5211/X5212/X5213/X5214/X5215/X5217/X5220/ |
| | | X5221/X5222/X5225/X5227/X5229/X5230/X5231/X5232/X5233/X5237/X5238/ |
| | | X5239/X5240/X5242/X5243/X5244/X5245/X5248/X5249/X5252/X5253/X5254/ |
| | | X5255/X5256/X5257/X5258/X5259/X5260/X5282/X532/X6201/X6202/X6205/ |
| | | X6207/X6208/X6210/X6211/X6325/X6328/X6329/X6330/X6331/X6332/ |
| | | X6334/X6335/X6336/X6337/X6338/X6339/X6344/X6345/X6346/X6347/X6348/ |
| | | X6349/X6350/X6351/X6352/X6381/X6382/X6383/X6384/X6385/X6387/X6389/ |
| | | X6391/X6394/X6395/X6396/X6397/X6399/X6400/X6401/X6403/X6406/ |
| | | X6407/X6408/X6409/X6413/X6414/X6415/X6416/X6418/X6419/X6420/X6422/ |
| | | X6424/X6426/X6427/X6428/X6430/X6431/X6432/X6433/X6435/X6436/X6437/ |
| | | X6438/X6439/X6440/X6442/X6443/X6461/X6503/X7412/X745/X746/X747/ |
| | | X748/X750/X7507/X751/X7510/X7511/X7513/X7514/X7515/X7516/X7517/ |
| | | X7518/X7519/X7520/X753/X7537/X7538/X754/X7540/X7541/X7544/X7547/ |
| | | X755/X7550/X7551/X7553/X7556/X7557/X7558/X756/X7560/X7563/X7564/ |
| | | X7568/X7570/X7571/X7572/X7573/X7575/X7576/X7577/X7578/X7579/X7580/ |
| | | X7581/X7582/X7584/X7585/X7586/X7587/X7589/X7662/X789/X792/X795/ |
| | | X823/X824/X854/X855/X858/X8631/X8632/X8645/X8647/X8654/X8655/ |
| | | X8657/X8660/X8661/X8664/X8665/X8666/X8667/X8668/X8669/X8670/X8673/ |
| | | X8674/X8716/X901/X903/X9031/X93/X94/X9624/X9630/X9631/X9632/X9634/X9635 |
| ASN_seq_SS_ssproH | −Inf | X117/X1219/X1222/X1223/X1224/X1225/X1226/X1228/X1229/X1230/X1231/ |
| | | X1232/X1233/X1234/X1235/X1236/X1237/X1238/X1239/X1240/X1267/X13/ |
| | | X1354/X1431/X1435/X1898/X1899/X1901/X1902/X1903/X1904/X1905/X1906/ |
| | | X1907/X1908/X1910/X1911/X1912/X1914/X1915/X1916/X1917/X1918/X1919/ |
| | | X1920/X1921/X1922/X1923/X1924/X1925/X1953/X2035/X2146/X2148/ |
| | | X215/X2151/X2154/X2157/X216/X217/X218/X26/X263/X2769/X2770/X2771/ |
| | | X2773/X2774/X2775/X2777/X2779/X2780/X2781/X2782/X2783/X2785/X2786/ |
| | | X2789/X2790/X2791/X2792/X2793/X2794/X2795/X2796/X2797/X2798/ |
| | | X2799/X2828/X2922/X2923/X2927/X3036/X3038/X3039/X3042/X3046/X3050/ |
| | | X3053/X32/X3813/X3814/X3815/X3816/X3817/X3819/X3820/X3823/X3825/ |
| | | X3826/X3827/X3829/X3830/X3831/X3832/X3833/X3834/X3835/X3836/X3966/ |
| | | X3970/X3972/X3974/X3976/X4067/X4070/X4074/X4079/X4083/X4087/ |
| | | X4090/X4097/X4105/X424/X425/X426/X427/X428/X429/X430/X45/X4978/X4979/ |
| | | X4980/X4982/X4984/X4985/X4988/X4989/X4990/X4991/X508/X509/X5120/ |
| | | X5123/X5125/X5127/X5129/X5134/X5200/X5211/X5213/X5220/X5229/ |
| | | X5233/X5237/X5242/X5253/X532/X6207/X6208/X6211/X6325/X6328/X6334/ |
| | | X6336/X6384/X6394/X6400/X6406/X6418/X6426/X6433/X7/X744/X746/X747/ |
| | | X748/X749/X750/X751/X7510/X752/X753/X754/X755/X7556/X756/X7563/ |
| | | X7575/X851/X852/X8660/X901/X92/X93/X94 |
| ASN_struct_aa_A | −Inf | X113/X1344/X1345/X1346/X1347/X1961/X2018/X2024/X2042/X2043/X2044/ |
| | | X2045/X258/X2855/X2902/X2906/X2908/X2912/X2932/X2933/X2934/X3244/ |
| | | X356/X3907/X3909/X3949/X3951/X3956/X3960/X3980/X3981/X4315/X502/ |
| | | X5073/X5109/X5111/X5113/X5115/X5499/X6320/X6322/X6693/X843/X844 |
| ASN_struct_aa_C | −Inf | X4130/X5276/X6476/X7617 |
| ASN_struct_aa_D | −Inf | X10462/X10463/X10465/X10469/X10470/X10471/X10472/X10473/X10474/X10483/ |
| | | X10484/X10485/X10494/X10495/X11108/X11112/X11117/X11118/X11119/ |
| | | X11585/X1356/X1359/X1439/X1440/X170/X1961/X2055/X2057/X2063/ |
| | | X2120/X2161/X2162/X2163/X2166/X254/X2855/X2944/X2946/X2947/X2951/ |
| | | X2953/X2957/X2996/X2997/X3002/X3004/X3064/X3065/X3066/X3070/X3073/ |
| | | X3074/X3907/X3909/X3992/X3993/X3995/X3996/X3998/X3999/X4002/ |
| | | X4006/X4025/X4026/X4032/X4033/X4037/X4038/X4039/X4040/X4042/X4114/ |
| | | X4116/X4117/X4119/X4124/X4125/X4127/X4129/X4130/X4131/X4133/X4134/ |
| | | X489/X5073/X5141/X5142/X5144/X5145/X5147/X5149/X5151/X5154/X5161/ |
| | | X5164/X5165/X5169/X5170/X5171/X5172/X5173/X5174/X5178/X5179/ |
| | | X5180/X5181/X5262/X5264/X5265/X5267/X5268/X5269/X5270/X5273/X5275/ |
| | | X5276/X5277/X5279/X5280/X5283/X5284/X5285/X5286/X5287/X5288/X5290/ |
| | | X5293/X6344/X6345/X6346/X6348/X6349/X6352/X6353/X6355/X6356/ |
| | | X6357/X6358/X6359/X6363/X6364/X6365/X6366/X6367/X6368/X6369/X6445/ |
| | | X6447/X6448/X6449/X6450/X6452/X6453/X6454/X6455/X6456/X6457/X6458/ |
| | | X6459/X6462/X6463/X6465/X6466/X6467/X6468/X6469/X6471/X6475/ |
| | | X6476/X6477/X6479/X6480/X6481/X6482/X6483/X6484/X6490/X6491/X7517/ |
| | | X7519/X7520/X7521/X7522/X7524/X7525/X7526/X7527/X7528/X7529/X7530/ |
| | | X7591/X7593/X7594/X7595/X7596/X7597/X7598/X7599/X7600/X7602/ |
| | | X7603/X7604/X7605/X7606/X7607/X7608/X7609/X7610/X7611/X7615/X7616/ |
| | | X7617/X7618/X7619/X7621/X7623/X7625/X7626/X7627/X7628/X7629/X7635/ |
| | | X7636/X7637/X7638/X7640/X7643/X7651/X7652/X855/X8633/X8634/ |
| | | X8635/X8636/X8676/X8678/X8680/X8681/X8682/X8683/X8684/X8685/X8686/ |
| | | X8687/X8688/X8689/X8692/X8693/X8694/X8695/X8696/X8697/X8698/X8699/ |
| | | X8700/X8701/X8707/X8708/X8709/X8710/X8711/X8712/X8713/X8715/ |
| | | X8716/X8717/X8720/X8728/X8729/X8730/X8731/X8742/X907/X9639/X9640/ |
| | | X9641/X9643/X9644/X9645/X9646/X9647/X9648/X9649/X9650/X9651/X9652/ |
| | | X9658/X9659/X9660/X9661/X9662/X9663/X9672/X9673/X9674/X9675/X9676/ |
| | | X9677/X9678/X9689/X9690 |
| ASN_struct_aa_E | −Inf | X1294/X1295/X1296/X1993/X1995/X1996/X245/X27/X2885/X2887/X470/X80/ |
| | | X801/X802 |
| ASN_struct_aa_F | −Inf | X10469/X10472/X10473/X10483/X10495/X11117/X1439/X170/X2161/X2163/ |
| | | X3064/X3066/X3074/X4116/X4117/X4125/X4129/X4133/X4134/X5262/X5265/ |
| | | X5267/X5268/X5270/X5275/X5279/X5280/X5284/X5285/X5286/X5287/X5290/ |
| | | X6445/X6447/X6448/X6450/X6453/X6456/X6457/X6459/X6465/X6466/ |
| | | X6467/X6468/X6471/X6475/X6479/X6480/X6481/X6482/X6483/X7594/X7597/ |
| | | X7598/X7600/X7602/X7603/X7605/X7606/X7609/X7610/X7616/X7623/X7625/ |
| | | X7626/X7627/X7628/X7635/X7636/X7637/X7640/X7643/X8680/X8681/ |
| | | X8683/X8684/X8687/X8688/X8692/X8695/X8696/X8698/X8699/X8708/X8710/ |
| | | X8711/X8717/X8720/X8728/X8729/X8730/X9643/X9646/X9647/X9649/X9650/ |
| | | X9658/X9661/X9662/X9673/X9675/X9676/X9690 |
| ASN_struct_aa_G | −Inf | X5287/X6468/X6483/X7628/X7637/X8711/X8730/X9676 |
| ASN_struct_aa_H | −Inf | X10462/X10463/X10465/X10469/X10470/X10471/X10472/X10473/X10474/X10483/ |
| | | X10484/X10485/X10494/X10495/X11108/X11112/X11117/X11118/X11119/ |
| | | X11585/X1356/X1357/X1359/X1360/X1439/X1440/X170/X2055/X2057/ |
| | | X2058/X2059/X2061/X2063/X2120/X214/X2161/X2162/X2163/X2166/X254/ |
| | | X2944/X2945/X2946/X2947/X2949/X2951/X2953/X2954/X2957/X2996/X2997/ |
| | | X3002/X3004/X3064/X3065/X3066/X3070/X3073/X3074/X3991/X3992/X3993/ |
| | | X3995/X3996/X3998/X3999/X4002/X4004/X4006/X4025/X4026/X4032/ |
| | | X4033/X4037/X4038/X4039/X4040/X4042/X4114/X4116/X4117/X4119/X4124/ |
| | | X4125/X4127/X4129/X4130/X4131/X4133/X4134/X489/X510/X5141/X5142/ |
| | | X5144/X5145/X5146/X5147/X5149/X5151/X5154/X5161/X5164/X5165/X5169/ |
| | | X5170/X5171/X5172/X5173/X5174/X5178/X5179/X5180/X5181/X5262/ |
| | | X5264/X5265/X5267/X5268/X5269/X5270/X5273/X5275/X5276/X5277/X5279/ |
| | | X5280/X5283/X5284/X5285/X5286/X5287/X5288/X5290/X5293/X6344/X6345/ |
| | | X6346/X6348/X6349/X6352/X6353/X6355/X6356/X6357/X6358/X6359/ |
| | | X6363/X6364/X6365/X6366/X6367/X6368/X6369/X6445/X6447/X6448/X6449/ |
| | | X6450/X6452/X6453/X6454/X6455/X6456/X6457/X6458/X6459/X6462/X6463/ |
| | | X6465/X6466/X6467/X6468/X6469/X6471/X6475/X6476/X6477/X6479/ |
| | | X6480/X6481/X6482/X6483/X6484/X6490/X6491/X7517/X7519/X7520/X7521/ |
| | | X7522/X7524/X7525/X7526/X7527/X7528/X7529/X7530/X7591/X7593/X7594/ |
| | | X7595/X7596/X7597/X7598/X7599/X7600/X7602/X7603/X7604/X7605/ |
| | | X7606/X7607/X7608/X7609/X7610/X7611/X7615/X7616/X7617/X7618/X7619/ |
| | | X7621/X7623/X7625/X7626/X7627/X7628/X7629/X7635/X7636/X7637/X7638/ |
| | | X7640/X7643/X7651/X7652/X84/X855/X856/X8633/X8634/X8635/X8636/ |
| | | X8676/X8678/X8680/X8681/X8682/X8683/X8684/X8685/X8686/X8687/X8688/ |
| | | X8689/X8692/X8693/X8694/X8695/X8696/X8697/X8698/X8699/X8700/ |
| | | X8701/X8707/X8708/X8709/X8710/X8711/X8712/X8713/X8715/X8716/X8717/ |
| | | X8720/X8728/X8729/X8730/X8731/X8742/X907/X9639/X9640/X9641/X9643/ |
| | | X9644/X9645/X9646/X9647/X9648/X9649/X9650/X9651/X9652/X9658/ |
| | | X9659/X9660/X9661/X9662/X9663/X9672/X9673/X9674/X9675/X9676/X9677/ |
| | | X9678/X9689/X9690 |
| ASN_struct_aa_I | −Inf | X1316/X1317/X1318/X1320/X1321/X1322/X2011/X2012/X2013/X2015/X2016/ |
| | | X2017/X2021/X2022/X2023/X253/X2895/X2899/X2900/X2901/X2904/X2910/ |
| | | X2913/X2914/X3946/X3953/X3954/X3955/X3958/X3959/X485/X487/X5106/ |
| | | X5107/X5108/X822/X823/X824 |
| ASN_struct_aa_L | −Inf | X238/X356 |
| ASN_struct_aa_M | −Inf | X10459/X10494/X1220/X1221/X1222/X1223/X1225/X1227/X1228/X1229/X1230/ |
| | | X1231/X1232/X1233/X1234/X1235/X1236/X1237/X1238/X1239/X1240/ |
| | | X1356/X1357/X1358/X1359/X1360/X1361/X1362/X1363/X1364/X1365/X1382/ |
| | | X1431/X1432/X1433/X1435/X1437/X1557/X1895/X1896/X1897/X1898/X1900/ |
| | | X1901/X1902/X1903/X1904/X1905/X1906/X1907/X1909/X1910/X1911/ |
| | | X1912/X1913/X1914/X1915/X1916/X1917/X1918/X1919/X1920/X1921/X1922/ |
| | | X1923/X1924/X1925/X2035/X2036/X2055/X2056/X2057/X2058/X2059/X2060/ |
| | | X2061/X2062/X2063/X2064/X2065/X2066/X2067/X2068/X2069/X2081/ |
| | | X2120/X214/X2145/X2146/X2148/X2149/X215/X2150/X2151/X2152/X2154/ |
| | | X2157/X2160/X217/X218/X2331/X2332/X265/X2764/X2765/X2766/X2767/X2768/ |
| | | X2769/X2770/X2772/X2773/X2774/X2775/X2776/X2777/X2778/X2779/ |
| | | X2780/X2781/X2782/X2783/X2784/X2785/X2786/X2787/X2788/X2789/X2790/ |
| | | X2791/X2792/X2793/X2794/X2795/X2796/X2797/X2798/X2799/X29/X2922/ |
| | | X2923/X2924/X2925/X2926/X2927/X2928/X2944/X2945/X2946/X2947/X2948/ |
| | | X2949/X2950/X2951/X2952/X2953/X2954/X2955/X2956/X2957/X2958/ |
| | | X2959/X2960/X2961/X2962/X2965/X2996/X2997/X3002/X3004/X3032/X3033/ |
| | | X3034/X3035/X3036/X3038/X3039/X3040/X3042/X3044/X3045/X3046/X3050/ |
| | | X3051/X3052/X3053/X3056/X3059/X3060/X3061/X3062/X3072/X32/X3285/ |
| | | X3286/X3287/X3293/X3803/X3804/X3805/X3806/X3807/X3808/X3810/ |
| | | X3811/X3812/X3813/X3814/X3815/X3816/X3817/X3818/X3819/X3820/X3821/ |
| | | X3822/X3823/X3824/X3825/X3826/X3827/X3828/X3829/X3830/X3831/X3832/ |
| | | X3833/X3834/X3835/X3836/X3966/X3967/X3968/X3969/X3970/X3972/ |
| | | X3973/X3974/X3975/X3976/X3977/X3978/X3991/X3992/X3993/X3994/X3995/ |
| | | X3996/X3997/X3998/X3999/X4000/X4001/X4002/X4003/X4004/X4005/X4006/ |
| | | X4007/X4008/X4009/X4025/X4026/X4032/X4033/X4037/X4038/X4039/ |
| | | X4040/X4042/X4066/X4067/X4069/X4070/X4071/X4072/X4073/X4074/X4075/ |
| | | X4076/X4078/X4079/X4080/X4081/X4082/X4083/X4087/X4088/X4089/X4090/ |
| | | X4093/X4095/X4096/X4097/X4098/X4099/X4102/X4103/X4104/X4105/ |
| | | X4108/X4109/X4111/X4112/X4113/X4123/X4132/X425/X426/X428/X429/X430/ |
| | | X4373/X4374/X4376/X4378/X4384/X4389/X4392/X4394/X4969/X4970/ |
| | | X4971/X4973/X4974/X4975/X4976/X4977/X4978/X4979/X4980/X4981/X4982/ |
| | | X4983/X4984/X4985/X4986/X4987/X4988/X4989/X4990/X4991/X510/X511/ |
| | | X5119/X512/X5120/X5123/X5124/X5125/X5127/X5128/X5129/X5130/X5131/ |
| | | X5132/X5133/X5134/X5135/X5141/X5142/X5143/X5144/X5145/X5146/X5147/ |
| | | X5148/X5149/X5150/X5151/X5152/X5153/X5154/X5155/X5161/X5164/ |
| | | X5165/X5169/X5170/X5171/X5172/X5173/X5174/X5178/X5179/X5180/X5181/ |
| | | X5200/X5201/X5202/X5203/X5204/X5205/X5206/X5207/X5208/X5210/X5211/ |
| | | X5212/X5213/X5214/X5215/X5217/X5218/X5219/X5220/X5221/X5222/ |
| | | X5225/X5227/X5228/X5229/X5230/X5231/X5232/X5233/X5237/X5238/X5239/ |
| | | X5240/X5241/X5242/X5243/X5244/X5245/X5248/X5249/X5252/X5253/X5254/ |
| | | X5255/X5256/X5257/X5258/X5259/X5260/X5278/X5282/X5289/X5293/ |
| | | X5309/X5310/X532/X5564/X5565/X5566/X5570/X5575/X5580/X5583/X5586/ |
| | | X5588/X5590/X5594/X6200/X6201/X6202/X6204/X6205/X6206/X6207/X6208/ |
| | | X6210/X6211/X6325/X6328/X6329/X6330/X6331/X6332/X6334/X6335/ |
| | | X6336/X6337/X6338/X6339/X6344/X6345/X6346/X6347/X6348/X6349/X6350/ |
| | | X6351/X6352/X6353/X6355/X6356/X6357/X6358/X6359/X6363/X6364/X6365/ |
| | | X6366/X6367/X6368/X6369/X6381/X6382/X6383/X6384/X6385/X6386/ |
| | | X6387/X6388/X6389/X6391/X6392/X6393/X6394/X6395/X6396/X6397/X6399/ |
| | | X6400/X6401/X6403/X6404/X6405/X6406/X6407/X6408/X6409/X6413/X6414/ |
| | | X6415/X6416/X6417/X6418/X6419/X6420/X6422/X6424/X6425/X6426/ |
| | | X6427/X6428/X6430/X6431/X6432/X6433/X6434/X6435/X6436/X6437/X6438/ |
| | | X6439/X6440/X6441/X6442/X6443/X6461/X6462/X6470/X6478/X6490/X6491/ |
| | | X6503/X6506/X6507/X6760/X6762/X6764/X6767/X6772/X6773/X6777/ |
| | | X6787/X6793/X6796/X6798/X6812/X7409/X7412/X745/X746/X747/X748/X750/ |
| | | X7507/X751/X7510/X7511/X7513/X7514/X7515/X7516/X7517/X7518/X7519/ |
| | | X7520/X7521/X7522/X7524/X7525/X7526/X7527/X7528/X7529/X753/X7530/ |
| | | X7537/X7538/X7539/X754/X7540/X7541/X7544/X7545/X7547/X7549/ |
| | | X755/X7550/X7551/X7553/X7554/X7555/X7556/X7557/X7558/X756/X7560/ |
| | | X7561/X7562/X7563/X7564/X7566/X7568/X7570/X7571/X7572/X7573/X7574/ |
| | | X7575/X7576/X7577/X7578/X7579/X7580/X7581/X7582/X7583/X7584/X7585/ |
| | | X7586/X7587/X7589/X7615/X7618/X7620/X7621/X7639/X7651/X7652/ |
| | | X7662/X7664/X7665/X7912/X7914/X7916/X7922/X7926/X7931/X7946/X7948/ |
| | | X7961/X84/X854/X855/X856/X857/X858/X859/X8631/X8632/X8633/X8634/ |
| | | X8635/X8636/X8642/X8645/X8646/X8647/X8648/X8651/X8654/X8655/X8657/ |
| | | X8658/X8659/X8660/X8661/X8664/X8665/X8666/X8667/X8668/X8669/ |
| | | X8670/X8673/X8674/X8707/X8709/X8712/X8714/X8715/X8716/X8742/X8977/ |
| | | X8979/X8990/X90/X9000/X901/X903/X9031/X9041/X9043/X93/X94/X9624/ |
| | | X9625/X9630/X9631/X9632/X9634/X9635/X9672/X9674/X9677/X9689/X9903/ |
| | | X9932 |
| ASN_struct_aa_P | −Inf | X10494/X1356/X1359/X2055/X2057/X2063/X2120/X2944/X2946/X2951/X2953/ |
| | | X2957/X2996/X2997/X3002/X3004/X3992/X3993/X3995/X3996/X3998/X4002/ |
| | | X4006/X4025/X4026/X4032/X4033/X4037/X4038/X4039/X4040/X4042/ |
| | | X5141/X5142/X5144/X5145/X5147/X5151/X5154/X5161/X5164/X5165/X5169/ |
| | | X5170/X5171/X5172/X5173/X5174/X5178/X5179/X5180/X5181/X5287/X5293/ |
| | | X6344/X6346/X6348/X6349/X6352/X6353/X6355/X6356/X6357/X6358/ |
| | | X6359/X6363/X6364/X6365/X6366/X6367/X6368/X6369/X6462/X6468/X6483/ |
| | | X6490/X6491/X7517/X7519/X7520/X7521/X7522/X7524/X7525/X7526/X7527/ |
| | | X7528/X7529/X7530/X7615/X7618/X7621/X7628/X7637/X7651/X7652/ |
| | | X855/X8633/X8634/X8635/X8636/X8707/X8709/X8711/X8712/X8715/X8716/ |
| | | X8730/X8742/X9672/X9674/X9676/X9677/X9689 |
| ASN_struct_aa_Q | −Inf | X10454/X10457/X10458/X10459/X10460/X10461/X10469/X10472/X10473/X10483/ |
| | | X10495/X10656/X10670/X10679/X10695/X10700/X10702/X11117/X11249/ |
| | | X11422/X1220/X1221/X1222/X1223/X1225/X1227/X1228/X1229/X1230/ |
| | | X1231/X1232/X1233/X1234/X1235/X1236/X1237/X1238/X1239/X1431/X1432/ |
| | | X1433/X1435/X1437/X1439/X1557/X1895/X1896/X1897/X1898/X1900/ |
| | | X1901/X1902/X1903/X1904/X1905/X1906/X1907/X1909/X1910/X1911/X1912/ |
| | | X1913/X1915/X1916/X1917/X1918/X1920/X1921/X1922/X1923/X1924/X1925/ |
| | | X2035/X2036/X2145/X2146/X2148/X2149/X215/X2150/X2151/X2152/X2154/ |
| | | X2157/X2160/X2161/X2163/X217/X218/X2331/X2332/X2764/X2765/X2766/ |
| | | X2767/X2768/X2769/X2770/X2772/X2773/X2774/X2775/X2776/X2778/ |
| | | X2779/X2780/X2781/X2782/X2783/X2784/X2785/X2786/X2787/X2788/X2789/ |
| | | X2790/X2791/X2792/X2793/X2794/X2795/X2796/X2797/X2798/X2922/X2923/ |
| | | X2924/X2925/X2926/X2927/X2928/X3032/X3033/X3034/X3035/X3036/ |
| | | X3038/X3039/X3040/X3042/X3044/X3045/X3046/X3050/X3051/X3052/X3053/ |
| | | X3056/X3059/X3060/X3061/X3062/X3064/X3066/X3074/X32/X3285/X3286/ |
| | | X3287/X3293/X3294/X3299/X3332/X3803/X3804/X3805/X3806/X3807/X3808/ |
| | | X3809/X3810/X3811/X3812/X3813/X3814/X3815/X3816/X3817/X3818/ |
| | | X3819/X3820/X3821/X3822/X3823/X3824/X3825/X3826/X3827/X3828/X3830/ |
| | | X3831/X3832/X3834/X3835/X3836/X3966/X3967/X3968/X3969/X3970/X3972/ |
| | | X3973/X3974/X3975/X3976/X3977/X3978/X4066/X4067/X4069/X4070/ |
| | | X4071/X4072/X4073/X4074/X4075/X4076/X4078/X4079/X4080/X4081/X4082/ |
| | | X4083/X4087/X4088/X4089/X4090/X4093/X4095/X4096/X4097/X4098/X4099/ |
| | | X4102/X4103/X4104/X4105/X4108/X4109/X4110/X4111/X4112/X4113/ |
| | | X4116/X4117/X4125/X4129/X4132/X4133/X4134/X4158/X425/X426/X428/ |
| | | X429/X430/X4373/X4374/X4376/X4378/X4380/X4384/X4385/X4389/X4391/ |
| | | X4392/X4394/X4397/X4442/X4968/X4969/X4970/X4971/X4972/X4973/X4974/ |
| | | X4975/X4976/X4977/X4978/X4979/X4980/X4981/X4983/X4984/X4985/X4986/ |
| | | X4987/X4988/X4989/X4990/X4991/X5119/X5120/X5123/X5124/X5125/ |
| | | X5127/X5128/X5129/X5130/X5131/X5132/X5133/X5134/X5135/X5200/X5201/ |
| | | X5202/X5203/X5204/X5205/X5206/X5207/X5208/X5210/X5211/X5212/X5213/ |
| | | X5214/X5215/X5217/X5218/X5219/X5220/X5221/X5222/X5225/X5227/ |
| | | X5228/X5229/X5230/X5231/X5232/X5233/X5237/X5238/X5239/X5240/X5241/ |
| | | X5242/X5243/X5244/X5245/X5248/X5249/X5250/X5252/X5253/X5254/X5255/ |
| | | X5256/X5257/X5258/X5259/X5260/X5262/X5265/X5267/X5268/X5270/ |
| | | X5275/X5278/X5279/X5280/X5284/X5285/X5286/X5287/X5289/X5290/X5309/ |
| | | X5310/X5315/X532/X5564/X5565/X5566/X5567/X5568/X5570/X5572/X5575/ |
| | | X5577/X5579/X5580/X5583/X5585/X5586/X5588/X5590/X5591/X5594/X5596/ |
| | | X5598/X5604/X5659/X6199/X6200/X6201/X6202/X6203/X6204/X6205/ |
| | | X6206/X6207/X6208/X6209/X6210/X6211/X6325/X6328/X6329/X6330/X6331/ |
| | | X6332/X6334/X6335/X6336/X6337/X6338/X6339/X6381/X6382/X6383/X6384/ |
| | | X6385/X6386/X6387/X6388/X6389/X6391/X6392/X6393/X6394/X6395/ |
| | | X6396/X6397/X6399/X6400/X6401/X6402/X6403/X6404/X6405/X6406/X6407/ |
| | | X6408/X6409/X6413/X6414/X6415/X6416/X6417/X6418/X6419/X6420/X6422/ |
| | | X6424/X6425/X6426/X6427/X6428/X6430/X6431/X6432/X6433/X6434/ |
| | | X6435/X6436/X6437/X6438/X6439/X6440/X6441/X6442/X6443/X6445/X6447/ |
| | | X6448/X6450/X6453/X6456/X6457/X6459/X6465/X6466/X6467/X6468/X6470/ |
| | | X6471/X6475/X6478/X6479/X6480/X6481/X6482/X6483/X6503/X6506/ |
| | | X6507/X6510/X6760/X6761/X6762/X6763/X6764/X6765/X6767/X6769/X6771/ |
| | | X6772/X6773/X6775/X6777/X6780/X6782/X6784/X6787/X6789/X6791/X6793/ |
| | | X6795/X6796/X6798/X6805/X6807/X6812/X7408/X7409/X7411/X7412/ |
| | | X745/X746/X747/X748/X750/X7507/X751/X7510/X7511/X7513/X7514/X7515/ |
| | | X7516/X753/X7537/X7538/X7539/X754/X7540/X7541/X7542/X7543/X7544/ |
| | | X7545/X7546/X7547/X7549/X755/X7550/X7551/X7552/X7553/X7554/X7555/ |
| | | X7556/X7557/X7558/X756/X7560/X7561/X7562/X7563/X7564/X7566/X7568/ |
| | | X7569/X7570/X7571/X7572/X7573/X7574/X7575/X7576/X7577/X7578/ |
| | | X7579/X7580/X7581/X7582/X7583/X7584/X7585/X7586/X7587/X7588/X7589/ |
| | | X7594/X7597/X7598/X7600/X7602/X7603/X7605/X7606/X7609/X7610/X7616/ |
| | | X7620/X7623/X7625/X7626/X7627/X7628/X7635/X7636/X7637/X7639/ |
| | | X7640/X7643/X7662/X7664/X7665/X7911/X7912/X7913/X7914/X7915/X7916/ |
| | | X7918/X7920/X7922/X7925/X7926/X7928/X7930/X7931/X7936/X7942/X7945/ |
| | | X7946/X7948/X7955/X7957/X7961/X7963/X7965/X7967/X7972/X7974/ |
| | | X7978/X7992/X8631/X8632/X8640/X8641/X8642/X8643/X8644/X8645/X8646/ |
| | | X8647/X8648/X8649/X8651/X8652/X8653/X8654/X8655/X8656/X8657/X8658/ |
| | | X8659/X8660/X8661/X8662/X8663/X8664/X8665/X8666/X8667/X8668/ |
| | | X8669/X8670/X8671/X8672/X8673/X8674/X8675/X8680/X8681/X8683/X8684/ |
| | | X8687/X8688/X8692/X8695/X8696/X8698/X8699/X8708/X8710/X8711/X8714/ |
| | | X8717/X8720/X8728/X8729/X8730/X8975/X8976/X8977/X8979/X8980/ |
| | | X8985/X8987/X8989/X8990/X8995/X9000/X9003/X9008/X901/X9014/X9016/ |
| | | X9018/X9020/X9025/X9027/X903/X9031/X9036/X9038/X9041/X9043/X9046/ |
| | | X93/X94/X9619/X9620/X9621/X9622/X9623/X9624/X9625/X9627/X9628/ |
| | | X9629/X9630/X9631/X9632/X9633/X9634/X9635/X9636/X9637/X9643/X9646/ |
| | | X9647/X9649/X9650/X9658/X9661/X9662/X9673/X9675/X9676/X9690/X9897/ |
| | | X9898/X9903/X9909/X9912/X9917/X9926/X9932/X9937/X9939/X9943/ |
| | | X9948/X9950/X9967/X9969/X9972 |
| ASN_struct_aa_R | −Inf | X10462/X10463/X10465/X10469/X10470/X10471/X10472/X10473/X10474/X10483/ |
| | | X10484/X10485/X10494/X10495/X11108/X11112/X11117/X11118/X11119/ |
| | | X11585/X1356/X1359/X1439/X1440/X2055/X2057/X2063/X2120/X2161/ |
| | | X2162/X2163/X2166/X254/X2944/X2946/X2947/X2951/X2953/X2957/X2996/ |
| | | X2997/X3002/X3004/X3064/X3065/X3066/X3070/X3073/X3074/X3992/X3993/ |
| | | X3995/X3996/X3998/X3999/X4002/X4006/X4025/X4026/X4032/X4033/ |
| | | X4037/X4038/X4039/X4040/X4042/X4114/X4116/X4117/X4119/X4124/X4125/ |
| | | X4127/X4129/X4130/X4131/X4133/X4134/X489/X5141/X5142/X5144/X5145/ |
| | | X5147/X5149/X5151/X5154/X5161/X5164/X5165/X5169/X5170/X5171/ |
| | | X5172/X5173/X5174/X5178/X5179/X5180/X5181/X5262/X5264/X5265/X5267/ |
| | | X5268/X5269/X5270/X5273/X5275/X5276/X5277/X5279/X5280/X5283/X5284/ |
| | | X5285/X5286/X5287/X5288/X5290/X5293/X6344/X6345/X6346/X6348/ |
| | | X6349/X6352/X6353/X6355/X6356/X6357/X6358/X6359/X6363/X6364/X6365/ |
| | | X6366/X6367/X6368/X6369/X6445/X6447/X6448/X6449/X6450/X6452/X6453/ |
| | | X6454/X6455/X6456/X6457/X6458/X6459/X6462/X6463/X6465/X6466/ |
| | | X6467/X6468/X6469/X6471/X6475/X6476/X6477/X6479/X6480/X6481/X6482/ |
| | | X6483/X6484/X6490/X6491/X7517/X7519/X7520/X7521/X7522/X7524/X7525/ |
| | | X7526/X7527/X7528/X7529/X7530/X7591/X7593/X7594/X7595/X7596/ |
| | | X7597/X7598/X7599/X7600/X7602/X7603/X7604/X7605/X7606/X7607/X7608/ |
| | | X7609/X7610/X7611/X7615/X7616/X7617/X7618/X7619/X7621/X7623/X7625/ |
| | | X7626/X7627/X7628/X7629/X7635/X7636/X7637/X7638/X7640/X7643/ |
| | | X7651/X7652/X855/X8633/X8634/X8635/X8636/X8676/X8678/X8680/X8681/ |
| | | X8682/X8683/X8684/X8685/X8686/X8687/X8688/X8689/X8692/X8693/X8694/ |
| | | X8695/X8696/X8697/X8698/X8699/X8700/X8701/X8707/X8708/X8709/X8710/ |
| | | X8711/X8712/X8713/X8715/X8716/X8717/X8720/X8728/X8729/X8730/ |
| | | X8731/X8742/X907/X9639/X9640/X9641/X9643/X9644/X9645/X9646/X9647/ |
| | | X9648/X9649/X9650/X9651/X9652/X9658/X9659/X9660/X9661/X9662/X9663/ |
| | | X9672/X9673/X9674/X9675/X9676/X9677/X9678/X9689/X9690 |
| ASN_struct_aa_S | −Inf | X356 |
| ASN_struct_aa_T | −Inf | X1333/X1334/X1335/X1336/X2028/X2029/X2032/X2034/X210/X256/X2916/ |
| | | X2919/X2920/X31/X356/X3962/X496/X497/X835/X836/X837/X89/X9/X91 |
| ASN_struct_aa_V | −Inf | X10454/X10457/X10458/X10459/X10460/X10461/X10656/X10670/X10679/X10695/ |
| | | X10700/X10702/X11249/X11422/X1220/X1221/X1222/X1223/X1225/ |
| | | X1227/X1228/X1229/X1230/X1231/X1232/X1233/X1234/X1235/X1236/X1237/ |
| | | X1238/X1239/X1240/X1316/X1317/X1318/X1320/X1321/X1322/X1431/X1432/ |
| | | X1433/X1435/X1437/X1557/X1895/X1896/X1897/X1898/X1900/X1901/ |
| | | X1902/X1903/X1904/X1905/X1906/X1907/X1909/X1910/X1911/X1912/X1913/ |
| | | X1914/X1915/X1916/X1917/X1918/X1919/X1920/X1921/X1922/X1923/X1924/ |
| | | X1925/X2011/X2012/X2013/X2015/X2016/X2017/X2021/X2022/X2023/ |
| | | X2035/X2036/X2145/X2146/X2148/X2149/X215/X2150/X2151/X2152/X2154/ |
| | | X2157/X2160/X217/X218/X2331/X2332/X253/X2764/X2765/X2766/X2767/ |
| | | X2768/X2769/X2770/X2772/X2773/X2774/X2775/X2776/X2777/X2778/X2779/ |
| | | X2780/X2781/X2782/X2783/X2784/X2785/X2786/X2787/X2788/X2789/X2790/ |
| | | X2791/X2792/X2793/X2794/X2795/X2796/X2797/X2798/X2799/X2895/ |
| | | X2899/X2900/X2901/X2904/X2910/X2913/X2914/X2922/X2923/X2924/X2925/ |
| | | X2926/X2927/X2928/X3032/X3033/X3034/X3035/X3036/X3038/X3039/X3040/ |
| | | X3042/X3044/X3045/X3046/X3050/X3051/X3052/X3053/X3056/X3059/ |
| | | X3060/X3061/X3062/X32/X3285/X3286/X3287/X3293/X3294/X3299/X3332/ |
| | | X3803/X3804/X3805/X3806/X3807/X3808/X3809/X3810/X3811/X3812/X3813/ |
| | | X3814/X3815/X3816/X3817/X3818/X3819/X3820/X3821/X3822/X3823/X3824/ |
| | | X3825/X3826/X3827/X3828/X3829/X3830/X3831/X3832/X3833/X3834/ |
| | | X3835/X3836/X3946/X3953/X3954/X3955/X3958/X3959/X3966/X3967/X3968/ |
| | | X3969/X3970/X3972/X3973/X3974/X3975/X3976/X3977/X3978/X4066/X4067/ |
| | | X4069/X4070/X4071/X4072/X4073/X4074/X4075/X4076/X4078/X4079/ |
| | | X4080/X4081/X4082/X4083/X4087/X4088/X4089/X4090/X4093/X4095/X4096/ |
| | | X4097/X4098/X4099/X4102/X4103/X4104/X4105/X4108/X4109/X4110/X4111/ |
| | | X4112/X4113/X4158/X425/X426/X428/X429/X430/X4373/X4374/X4376/ |
| | | X4378/X4380/X4384/X4385/X4389/X4391/X4392/X4394/X4397/X4442/X485/ |
| | | X487/X4968/X4969/X4970/X4971/X4972/X4973/X4974/X4975/X4976/X4977/ |
| | | X4978/X4979/X4980/X4981/X4982/X4983/X4984/X4985/X4986/X4987/X4988/ |
| | | X4989/X4990/X4991/X5106/X5107/X5108/X5119/X5120/X5123/X5124/ |
| | | X5125/X5127/X5128/X5129/X5130/X5131/X5132/X5133/X5134/X5135/X5200/ |
| | | X5201/X5202/X5203/X5204/X5205/X5206/X5207/X5208/X5210/X5211/X5212/ |
| | | X5213/X5214/X5215/X5217/X5218/X5219/X5220/X5221/X5222/X5225/ |
| | | X5227/X5228/X5229/X5230/X5231/X5232/X5233/X5237/X5238/X5239/X5240/ |
| | | X5241/X5242/X5243/X5244/X5245/X5248/X5249/X5250/X5252/X5253/X5254/ |
| | | X5255/X5256/X5257/X5258/X5259/X5260/X5309/X5310/X5315/X532/ |
| | | X5564/X5565/X5566/X5567/X5568/X5570/X5572/X5575/X5577/X5579/X5580/ |
| | | X5583/X5585/X5586/X5588/X5590/X5591/X5594/X5596/X5598/X5604/X5659/ |
| | | X6199/X6200/X6201/X6202/X6203/X6204/X6205/X6206/X6207/X6208/ |
| | | X6209/X6210/X6211/X6325/X6328/X6329/X6330/X6331/X6332/X6334/X6335/ |
| | | X6336/X6337/X6338/X6339/X6381/X6382/X6383/X6384/X6385/X6386/X6387/ |
| | | X6388/X6389/X6391/X6392/X6393/X6394/X6395/X6396/X6397/X6399/ |
| | | X6400/X6401/X6402/X6403/X6404/X6405/X6406/X6407/X6408/X6409/X6413/ |
| | | X6414/X6415/X6416/X6417/X6418/X6419/X6420/X6422/X6424/X6425/X6426/ |
| | | X6427/X6428/X6430/X6431/X6432/X6433/X6434/X6435/X6436/X6437/ |
| | | X6438/X6439/X6440/X6441/X6442/X6443/X6503/X6506/X6507/X6510/X6760/ |
| | | X6761/X6762/X6763/X6764/X6765/X6767/X6769/X6771/X6772/X6773/X6775/ |
| | | X6777/X6780/X6782/X6784/X6787/X6789/X6791/X6793/X6795/X6796/ |
| | | X6798/X6805/X6807/X6812/X7408/X7409/X7411/X7412/X745/X746/X747/X748/ |
| | | X750/X7507/X751/X7510/X7511/X7513/X7514/X7515/X7516/X753/X7537/ |
| | | X7538/X7539/X754/X7540/X7541/X7542/X7543/X7544/X7545/X7546/X7547/ |
| | | X7549/X755/X7550/X7551/X7552/X7553/X7554/X7555/X7556/X7557/X7558/ |
| | | X756/X7560/X7561/X7562/X7563/X7564/X7566/X7568/X7569/X7570/ |
| | | X7571/X7572/X7573/X7574/X7575/X7576/X7577/X7578/X7579/X7580/X7581/ |
| | | X7582/X7583/X7584/X7585/X7586/X7587/X7588/X7589/X7662/X7664/X7665/ |
| | | X7911/X7912/X7913/X7914/X7915/X7916/X7918/X7920/X7922/X7925/ |
| | | X7926/X7928/X7930/X7931/X7936/X7942/X7945/X7946/X7948/X7955/X7957/ |
| | | X7961/X7963/X7965/X7967/X7972/X7974/X7978/X7992/X822/X823/X824/ |
| | | X8631/X8632/X8640/X8641/X8642/X8643/X8644/X8645/X8646/X8647/X8648/ |
| | | X8649/X8651/X8652/X8653/X8654/X8655/X8656/X8657/X8658/X8659/X8660/ |
| | | X8661/X8662/X8663/X8664/X8665/X8666/X8667/X8668/X8669/X8670/ |
| | | X8671/X8672/X8673/X8674/X8675/X8975/X8976/X8977/X8979/X8980/X8985/ |
| | | X8987/X8989/X8990/X8995/X9000/X9003/X9008/X901/X9014/X9016/X9018/ |
| | | X9020/X9025/X9027/X903/X9031/X9036/X9038/X9041/X9043/X9046/X93/ |
| | | X94/X9619/X9620/X9621/X9622/X9623/X9624/X9625/X9627/X9628/X9629/ |
| | | X9630/X9631/X9632/X9633/X9634/X9635/X9636/X9637/X9897/X9898/X9903/ |
| | | X9909/X9912/X9917/X9926/X9932/X9937/X9939/X9943/X9948/X9950/ |
| | | X9967/X9969/X9972 |
| ASN_struct_aa_W | −Inf | X10494/X1356/X1359/X2055/X2057/X2063/X2120/X2944/X2946/X2951/X2953/ |
| | | X2957/X2996/X2997/X3002/X3004/X3992/X3993/X3995/X3996/X3998/X4002/ |
| | | X4006/X4025/X4026/X4032/X4033/X4037/X4038/X4039/X4040/X4042/ |
| | | X5141/X5142/X5144/X5145/X5147/X5151/X5154/X5161/X5164/X5165/X5169/ |
| | | X5170/X5171/X5172/X5173/X5174/X5178/X5179/X5180/X5181/X5293/X6344/ |
| | | X6346/X6348/X6349/X6352/X6353/X6355/X6356/X6357/X6358/X6359/ |
| | | X6363/X6364/X6365/X6366/X6367/X6368/X6369/X6462/X6490/X6491/X7517/ |
| | | X7519/X7520/X7521/X7522/X7524/X7525/X7526/X7527/X7528/X7529/X7530/ |
| | | X7615/X7618/X7621/X7651/X7652/X855/X8633/X8634/X8635/X8636/ |
| | | X8707/X8709/X8712/X8715/X8716/X8742/X9672/X9674/X9677/X9689 |
| ASN_struct_aa_Y | −Inf | X1317/X1320/X1321/X1322/X2012/X2021/X2022/X2023/X253/X2910/X2913/ |
| | | X2914/X3958/X3959/X487/X823/X824 |
| ASN_struct_SS_dsspE | −Inf | X105/X1267/X1274/X1277/X1281/X1283/X1284/X1287/X1316/X1317/X1318/ |
| | | X1320/X1321/X1322/X1953/X1971/X1974/X1978/X1980/X2011/X2012/X2013/ |
| | | X2015/X2016/X2021/X2022/X2023/X239/X241/X253/X2828/X2857/X2868/ |
| | | X2895/X2899/X2900/X2904/X2910/X2913/X2914/X3865/X3946/X3953/X3954/ |
| | | X3958/X3959/X461/X462/X464/X485/X487/X5106/X5107/X787/X789/X792/ |
| | | X793/X795/X822/X823/X824 |
| ASN_struct_SS_dsspH | −Inf | X1189/X1191/X1193/X1194/X1867/X1869/X193/X194/X195/X196/X23/X2743/ |
| | | X397/X399/X400/X401/X402/X714/X715/X717/X718/X719/X75/X76 |
| ASN_struct_SS_dsspS | −Inf | X10462/X10463/X10465/X10469/X10470/X10471/X10472/X10473/X10474/X10483/ |
| | | X10484/X10485/X10494/X10495/X11108/X11112/X11117/X11118/X11119/ |
| | | X11585/X1356/X1357/X1359/X1360/X1439/X1440/X1961/X2055/X2057/ |
| | | X2058/X2059/X2061/X2063/X2120/X214/X2161/X2162/X2163/X2166/X254/ |
| | | X2855/X2944/X2945/X2946/X2947/X2949/X2951/X2953/X2954/X2957/X2996/ |
| | | X2997/X3002/X3004/X3064/X3065/X3066/X3070/X3073/X3074/X3907/ |
| | | X3909/X3991/X3992/X3993/X3995/X3996/X3998/X3999/X4002/X4004/X4006/ |
| | | X4025/X4026/X4032/X4033/X4037/X4038/X4039/X4040/X4042/X4114/X4116/ |
| | | X4117/X4119/X4124/X4125/X4127/X4129/X4130/X4131/X4133/X4134/ |
| | | X489/X510/X5141/X5142/X5144/X5145/X5146/X5147/X5149/X5151/X5154/ |
| | | X5161/X5164/X5165/X5169/X5170/X5171/X5172/X5173/X5174/X5178/X5179/ |
| | | X5180/X5181/X5262/X5264/X5265/X5267/X5268/X5269/X5270/X5273/X5275/ |
| | | X5276/X5277/X5279/X5280/X5283/X5284/X5285/X5286/X5287/X5288/ |
| | | X5290/X5293/X6344/X6345/X6346/X6348/X6349/X6352/X6353/X6355/X6356/ |
| | | X6357/X6358/X6359/X6363/X6364/X6365/X6366/X6367/X6368/X6369/X6445/ |
| | | X6447/X6448/X6449/X6450/X6452/X6453/X6454/X6455/X6456/X6457/ |
| | | X6458/X6459/X6462/X6463/X6465/X6466/X6467/X6468/X6469/X6471/X6475/ |
| | | X6476/X6477/X6479/X6480/X6481/X6482/X6483/X6484/X6490/X6491/X7517/ |
| | | X7519/X7520/X7521/X7522/X7524/X7525/X7526/X7527/X7528/X7529/ |
| | | X7530/X7591/X7593/X7594/X7595/X7596/X7597/X7598/X7599/X7600/X7602/ |
| | | X7603/X7604/X7605/X7606/X7607/X7608/X7609/X7610/X7611/X7615/X7616/ |
| | | X7617/X7618/X7619/X7621/X7623/X7625/X7626/X7627/X7628/X7629/ |
| | | X7635/X7636/X7637/X7638/X7640/X7643/X7651/X7652/X84/X855/X856/X8633/ |
| | | X8634/X8635/X8636/X8676/X8678/X8680/X8681/X8682/X8683/X8684/ |
| | | X8685/X8686/X8687/X8688/X8689/X8692/X8693/X8694/X8695/X8696/X8697/ |
| | | X8698/X8699/X8700/X8701/X8707/X8708/X8709/X8710/X8711/X8712/X8713/ |
| | | X8715/X8716/X8717/X8720/X8728/X8729/X8730/X8731/X8742/X907/X9639/ |
| | | X9640/X9641/X9643/X9644/X9645/X9646/X9647/X9648/X9649/X9650/ |
| | | X9651/X9652/X9658/X9659/X9660/X9661/X9662/X9663/X9672/X9673/X9674/ |
| | | X9675/X9676/X9677/X9678/X9689/X9690 |
| SER.THR_seq_aaAll_F | −Inf | X1/X17/X96 |
| SER.THR_seq_aaAll_G | −Inf | X208/X86 |
| SER.THR_seq_aaAll_H | −Inf | X208/X86 |
| SER.THR_seq_aaAll_L | −Inf | X119/X149/X208/X211/X27/X29/X32/X380/X456/X6/X781/X86/X90/X93 |
| SER.THR_seq_aaAll_M | −Inf | X208/X86 |
| SER.THR_seq_aaAll_Q | −Inf | X208/X86 |
| SER.THR_seq_aaAll_Y | −Inf | X208/X86 |
| SER.THR_seq_aaDown_F | −Inf | X1 |
| SER.THR_seq_aaDown_L | −Inf | X29 |
| SER.THR_seq_aaUp_F | −Inf | X1 |
| SER.THR_seq_aaUp_L | −Inf | X119/X149/X211/X27/X29/X32/X380/X456/X6/X781/X90/X93 |
| SER.THR_seq_aaUp_N | −Inf | X19/X2/X204/X28/X81/X87 |
| SER.THR_seq_aaUp_P | −Inf | X119/X1327/X1343/X1405/X1407/X149/X206/X2101/X211/X2111/X214/X221/ |
| | | X27/X2983/X32/X380/X381/X412/X423/X438/X456/X501/X6/X771/X781/ |
| | | X782/X84/X842/X88/X891/X90/X93 |
| SER.THR_struct_aa_L | −Inf | X119/X149/X211/X27/X29/X32/X380/X456/X6/X781/X90/X93 |
| SER.THR_struct_SS_dsspG | −Inf | X1/X17/X96 |
| ASN_seq_aaAll_A | 1.28 | X10425/X10426/X10427/X11095/X11096/X11581/X1162/X1163/X1164/X1165/ |
| | | X1167/X1169/X117/X1170/X1171/X1172/X1175/X1177/X1180/X1183/X1185/ |
| | | X1186/X13/X1354/X1355/X17/X1831/X1832/X1833/X1834/X1835/X1836/ |
| | | X1837/X184/X1840/X1841/X1842/X1844/X1848/X185/X1851/X1853/X1854/ |
| | | X1856/X1858/X190/X2054/X263/X2699/X2700/X2701/X2702/X2704/X2705/ |
| | | X2706/X2707/X2709/X2712/X2713/X2715/X2716/X2717/X2718/X2723/X2725/ |
| | | X2729/X2733/X2735/X2736/X3742/X3743/X3744/X3746/X3747/X3749/X3750/ |
| | | X3752/X3753/X3754/X3755/X3760/X3761/X3763/X3766/X3767/X3768/ |
| | | X3769/X3773/X3774/X3776/X3777/X3784/X383/X385/X387/X389/X391/X395/ |
| | | X45/X4915/X4916/X4917/X4919/X4920/X4921/X4922/X4923/X4924/X4926/ |
| | | X4927/X4929/X4932/X4933/X4934/X4935/X4939/X4940/X4941/X4942/X4947/ |
| | | X4948/X4950/X4952/X508/X509/X6159/X6160/X6161/X6162/X6163/X6164/ |
| | | X6165/X6167/X6168/X6170/X6171/X6173/X6174/X6175/X6176/X6181/ |
| | | X6182/X6184/X6185/X6186/X6187/X68/X694/X696/X697/X699/X701/X703/ |
| | | X706/X709/X71/X711/X7379/X7380/X7381/X7382/X7384/X7385/X7387/X7388/ |
| | | X7389/X7390/X7394/X7395/X7396/X7397/X7403/X7404/X851/X852/X853/ |
| | | X8542/X8543/X8544/X8545/X8546/X8547/X8548/X8552/X8553/X8555/X8556/ |
| | | X9566/X9567/X9568/X9569/X9570/X9571 |
| ASN_seq_aaAll_C | 1.28 | X1/X10469/X10472/X10473/X10483/X10494/X10495/X11117/X117/X1199/X1202/ |
| | | X1204/X13/X1354/X1355/X1356/X1357/X1358/X1359/X1360/X1361/X1362/ |
| | | X1363/X1364/X1365/X1439/X1440/X1479/X1876/X1878/X1880/X197/ |
| | | X2054/X2055/X2056/X2057/X2058/X2059/X2060/X2061/X2062/X2063/X2064/ |
| | | X2065/X2066/X2067/X2068/X2069/X2120/X214/X2161/X2162/X2163/X2166/ |
| | | X2212/X24/X254/X263/X265/X2748/X2751/X2753/X29/X2944/X2945/X2946/ |
| | | X2947/X2948/X2949/X2950/X2951/X2952/X2953/X2954/X2955/X2956/ |
| | | X2957/X2958/X2959/X2960/X2961/X2962/X2996/X2997/X3002/X3004/X3064/ |
| | | X3065/X3066/X3070/X3072/X3073/X3074/X3791/X3794/X3991/X3992/X3993/ |
| | | X3994/X3995/X3996/X3997/X3998/X3999/X4000/X4001/X4002/X4003/ |
| | | X4004/X4005/X4006/X4007/X4008/X4009/X4025/X4026/X403/X4032/X4033/ |
| | | X4037/X4038/X4039/X4040/X4042/X4114/X4116/X4117/X4119/X4123/X4124/ |
| | | X4125/X4127/X4129/X4130/X4132/X4133/X4134/X45/X489/X4961/X508/ |
| | | X509/X510/X511/X512/X5141/X5142/X5143/X5144/X5145/X5146/X5147/X5148/ |
| | | X5149/X5150/X5151/X5152/X5153/X5154/X5155/X5161/X5164/X5165/ |
| | | X5169/X5170/X5171/X5172/X5173/X5174/X5178/X5179/X5180/X5181/X5262/ |
| | | X5264/X5265/X5267/X5268/X5270/X5273/X5275/X5276/X5278/X5279/X5280/ |
| | | X5282/X5283/X5284/X5285/X5286/X5287/X5289/X5290/X5293/X6344/ |
| | | X6345/X6346/X6347/X6348/X6349/X6350/X6351/X6352/X6353/X6355/X6356/ |
| | | X6357/X6358/X6359/X6363/X6364/X6365/X6366/X6367/X6368/X6369/X6445/ |
| | | X6447/X6448/X6450/X6452/X6453/X6456/X6457/X6459/X6461/X6462/ |
| | | X6463/X6465/X6466/X6467/X6468/X6470/X6471/X6475/X6476/X6478/X6479/ |
| | | X6480/X6481/X6482/X6483/X6490/X6491/X68/X722/X725/X7517/X7518/ |
| | | X7519/X7520/X7521/X7522/X7524/X7525/X7526/X7527/X7528/X7529/X7530/ |
| | | X7593/X7594/X7597/X7598/X7600/X7602/X7603/X7605/X7606/X7609/X7610/ |
| | | X7615/X7616/X7617/X7618/X7620/X7621/X7623/X7625/X7626/X7627/ |
| | | X7628/X7635/X7636/X7637/X7639/X7640/X7643/X7651/X7652/X77/X84/X851/ |
| | | X852/X853/X854/X855/X856/X857/X858/X859/X8633/X8634/X8635/X8636/ |
| | | X8680/X8681/X8683/X8684/X8687/X8688/X8692/X8695/X8696/X8698/ |
| | | X8699/X8707/X8708/X8709/X8710/X8711/X8712/X8714/X8715/X8716/X8717/ |
| | | X8720/X8728/X8729/X8730/X8742/X90/X907/X9643/X9646/X9647/X9649/ |
| | | X9650/X9658/X9661/X9662/X9672/X9673/X9674/X9675/X9676/X9677/X9689/X9690 |
| ASN_seq_aaAll_D | 1.28 | X1167/X1168/X1177/X1180/X1182/X1183/X1185/X1266/X1267/X1839/X1850/ |
| | | X1851/X1853/X1858/X1860/X1861/X190/X1952/X1953/X2727/X2728/X2732/ |
| | | X2738/X2827/X2828/X3779/X3782/X3783/X3865/X387/X389/X395/X49/ |
| | | X4956/X701/X702/X706/X709/X71/X711 |
| ASN_seq_aaAll_E | 1.28 | X10469/X10472/X10473/X10483/X10495/X11117/X1206/X1207/X1266/X1267/ |
| | | X1439/X1883/X1952/X1953/X198/X2161/X2163/X25/X2827/X2828/X3064/ |
| | | X3066/X3074/X3865/X393/X406/X407/X4116/X4117/X4125/X4129/X4133/ |
| | | X4134/X5262/X5265/X5267/X5268/X5270/X5275/X5279/X5280/X5284/X5285/ |
| | | X5286/X5287/X5290/X6445/X6447/X6448/X6450/X6453/X6456/X6457/X6459/ |
| | | X6465/X6466/X6467/X6468/X6471/X6475/X6479/X6480/X6481/X6482/ |
| | | X6483/X727/X728/X729/X7594/X7597/X7598/X7600/X7602/X7603/X7605/X7606/ |
| | | X7609/X7610/X7616/X7623/X7625/X7626/X7627/X7628/X7635/X7636/ |
| | | X7637/X7640/X7643/X78/X8680/X8681/X8683/X8684/X8687/X8688/X8692/ |
| | | X8695/X8696/X8698/X8699/X8708/X8710/X8711/X8717/X8720/X8728/X8729/ |
| | | X8730/X9643/X9646/X9647/X9649/X9650/X9658/X9661/X9662/X9673/X9675/ |
| | | X9676/X9690 |
| ASN_seq_aaAll_F | 1.28 | X1316/X1317/X1318/X1320/X1321/X1322/X2011/X2012/X2013/X2015/X2016/ |
| | | X2021/X2022/X2023/X253/X2895/X2899/X2900/X2904/X2910/X2913/X2914/ |
| | | X3946/X3953/X3954/X3958/X3959/X485/X487/X5106/X5107/X822/X823/X824 |
| ASN_seq_aaAll_G | 1.28 | X1/X1164/X1169/X1172/X1184/X17/X1832/X1837/X184/X1840/X1843/X1852/ |
| | | X2703/X2705/X2708/X2713/X2734/X3745/X3762/X385/X4920/X68/X696/X703 |
| ASN_seq_aaAll_H | 1.28 | X1/X10425/X10426/X10427/X10459/X11095/X1162/X1163/X1164/X1165/X1167/ |
| | | X1169/X1170/X1171/X1172/X1177/X1180/X1186/X1206/X1207/X1208/ |
| | | X1219/X1220/X1221/X1222/X1223/X1224/X1225/X1226/X1227/X1228/X1229/ |
| | | X1230/X1231/X1232/X1233/X1234/X1235/X1236/X1237/X1238/X1239/X1240/ |
| | | X1266/X1267/X1316/X1317/X1318/X1320/X1321/X1322/X1431/X1432/ |
| | | X1433/X1435/X1437/X1557/X17/X1831/X1833/X1834/X1836/X1837/X184/X1840/ |
| | | X1841/X1842/X1844/X1848/X185/X1854/X1858/X1862/X1883/X1884/ |
| | | X1895/X1896/X1897/X1898/X1899/X190/X1900/X1901/X1902/X1903/X1904/ |
| | | X1905/X1906/X1907/X1908/X1909/X1910/X1911/X1912/X1913/X1914/X1915/ |
| | | X1916/X1917/X1918/X1919/X1920/X1921/X1922/X1923/X1924/X1925/X1952/ |
| | | X1953/X198/X2011/X2012/X2013/X2015/X2016/X2021/X2022/X2023/ |
| | | X2035/X2036/X2145/X2146/X2148/X2149/X215/X2150/X2151/X2152/X2154/ |
| | | X2157/X216/X2160/X217/X218/X2331/X2332/X25/X253/X26/X2699/X2700/ |
| | | X2701/X2702/X2706/X2707/X2709/X2712/X2713/X2715/X2716/X2717/X2718/ |
| | | X2725/X2729/X2736/X2756/X2764/X2765/X2766/X2767/X2768/X2769/X2770/ |
| | | X2771/X2772/X2773/X2774/X2775/X2776/X2777/X2778/X2779/X2780/ |
| | | X2781/X2782/X2783/X2784/X2785/X2786/X2787/X2788/X2789/X2790/X2791/ |
| | | X2792/X2793/X2794/X2795/X2796/X2797/X2798/X2799/X2827/X2828/X2895/ |
| | | X2899/X2900/X2904/X2910/X2913/X2914/X2922/X2923/X2924/X2925/ |
| | | X2926/X2927/X2928/X3032/X3033/X3034/X3035/X3036/X3038/X3039/X3040/ |
| | | X3042/X3044/X3045/X3046/X3050/X3051/X3052/X3053/X3056/X3059/X3060/ |
| | | X3061/X3062/X32/X3285/X3286/X3287/X3293/X3742/X3744/X3746/X3747/ |
| | | X3749/X3752/X3753/X3754/X3755/X3760/X3761/X3763/X3768/X3784/ |
| | | X3803/X3804/X3805/X3806/X3807/X3808/X3810/X3811/X3812/X3813/X3814/ |
| | | X3815/X3816/X3817/X3818/X3819/X3820/X3821/X3822/X3823/X3824/X3825/ |
| | | X3826/X3827/X3828/X3829/X383/X3830/X3831/X3832/X3833/X3834/ |
| | | X3835/X3836/X385/X389/X391/X3946/X3947/X395/X3953/X3954/X3958/X3959/ |
| | | X396/X3966/X3967/X3968/X3969/X3970/X3972/X3973/X3974/X3975/X3976/ |
| | | X3977/X3978/X406/X4066/X4067/X4069/X407/X4070/X4071/X4072/X4073/ |
| | | X4074/X4075/X4076/X4078/X4079/X4080/X4081/X4082/X4083/X4087/ |
| | | X4088/X4089/X4090/X4093/X4095/X4096/X4097/X4098/X4099/X4102/X4103/ |
| | | X4104/X4105/X4108/X4109/X4111/X4112/X4113/X424/X425/X426/X427/ |
| | | X428/X429/X430/X4373/X4374/X4376/X4378/X4384/X4389/X4392/X4394/ |
| | | X485/X487/X49/X4915/X4916/X4917/X4919/X4921/X4922/X4923/X4924/X4927/ |
| | | X4929/X4934/X4939/X4940/X4941/X4942/X4969/X4970/X4971/X4973/ |
| | | X4974/X4975/X4976/X4977/X4978/X4979/X4980/X4981/X4982/X4983/X4984/ |
| | | X4985/X4986/X4987/X4988/X4989/X4990/X4991/X5106/X5107/X5119/X5120/ |
| | | X5123/X5124/X5125/X5127/X5128/X5129/X5130/X5131/X5132/X5133/ |
| | | X5134/X5135/X5200/X5201/X5202/X5203/X5204/X5205/X5206/X5207/X5208/ |
| | | X5210/X5211/X5212/X5213/X5214/X5215/X5217/X5218/X5219/X5220/X5221/ |
| | | X5222/X5225/X5227/X5228/X5229/X5230/X5231/X5232/X5233/X5237/ |
| | | X5238/X5239/X5240/X5241/X5242/X5243/X5244/X5245/X5248/X5249/X5252/ |
| | | X5253/X5254/X5255/X5256/X5257/X5258/X5259/X5260/X5309/X5310/X532/ |
| | | X5564/X5565/X5566/X5570/X5575/X5580/X5583/X5586/X5588/X5590/X5594/ |
| | | X6/X6159/X6160/X6162/X6163/X6164/X6165/X6167/X6168/X6170/X6171/ |
| | | X6186/X6200/X6201/X6202/X6204/X6205/X6206/X6207/X6208/X6210/ |
| | | X6211/X6325/X6328/X6329/X6330/X6331/X6332/X6334/X6335/X6336/X6337/ |
| | | X6338/X6339/X6381/X6382/X6383/X6384/X6385/X6386/X6387/X6388/X6389/ |
| | | X6391/X6392/X6393/X6394/X6395/X6396/X6397/X6399/X6400/X6401/ |
| | | X6403/X6404/X6405/X6406/X6407/X6408/X6409/X6413/X6414/X6415/X6416/ |
| | | X6417/X6418/X6419/X6420/X6422/X6424/X6425/X6426/X6427/X6428/X6430/ |
| | | X6431/X6432/X6433/X6434/X6435/X6436/X6437/X6438/X6439/X6440/ |
| | | X6441/X6442/X6443/X6503/X6506/X6507/X6760/X6762/X6764/X6767/X6772/ |
| | | X6773/X6777/X6787/X6793/X6796/X6798/X68/X6812/X694/X696/X697/X699/ |
| | | X7/X701/X703/X706/X707/X71/X711/X727/X728/X729/X7379/X7380/X7381/ |
| | | X7382/X7384/X7385/X7387/X7388/X7389/X7390/X7409/X7412/X744/ |
| | | X745/X746/X747/X748/X749/X750/X7507/X751/X7510/X7511/X7513/X7514/ |
| | | X7515/X7516/X752/X753/X7537/X7538/X7539/X754/X7540/X7541/X7544/ |
| | | X7545/X7547/X7549/X755/X7550/X7551/X7553/X7554/X7555/X7556/X7557/ |
| | | X7558/X756/X7560/X7561/X7562/X7563/X7564/X7566/X7568/X7570/X7571/ |
| | | X7572/X7573/X7574/X7575/X7576/X7577/X7578/X7579/X7580/X7581/X7582/ |
| | | X7583/X7584/X7585/X7586/X7587/X7589/X7662/X7664/X7665/X78/X7912/ |
| | | X7914/X7916/X7922/X7926/X7931/X7946/X7948/X7961/X822/X823/X824/ |
| | | X8542/X8543/X8544/X8547/X8548/X8552/X8553/X8631/X8632/X8642/X8645/ |
| | | X8646/X8647/X8648/X8651/X8654/X8655/X8657/X8658/X8659/X8660/ |
| | | X8661/X8664/X8665/X8666/X8667/X8668/X8669/X8670/X8673/X8674/X8977/ |
| | | X8979/X8990/X9000/X901/X903/X9031/X9041/X9043/X92/X93/X94/X9566/ |
| | | X9567/X9568/X9569/X9571/X9624/X9625/X9630/X9631/X9632/X9634/X9635/ |
| | | X9903/X9932 |
| ASN_seq_aaAll_I | 1.28 | X1/X10494/X1172/X1356/X1358/X1359/X1361/X1365/X17/X1837/X1841/X1843/ |
| | | X1844/X2055/X2056/X2057/X2062/X2063/X2066/X2067/X2069/X2120/ |
| | | X2706/X2708/X2709/X2713/X2715/X2717/X2944/X2946/X2950/X2951/X2952/ |
| | | X2953/X2955/X2956/X2957/X2958/X2960/X2962/X2996/X2997/X3002/X3004/ |
| | | X3072/X3752/X3754/X3760/X3762/X3763/X3766/X3767/X3992/X3993/ |
| | | X3994/X3995/X3996/X3997/X3998/X4000/X4001/X4002/X4005/X4006/X4008/ |
| | | X4009/X4025/X4026/X4032/X4033/X4037/X4038/X4039/X4040/X4042/X4123/ |
| | | X4132/X4932/X4933/X4939/X4941/X511/X5141/X5142/X5143/X5144/X5145/ |
| | | X5147/X5150/X5151/X5152/X5153/X5154/X5155/X5161/X5164/X5165/ |
| | | X5169/X5170/X5171/X5172/X5173/X5174/X5178/X5179/X5180/X5181/X5278/ |
| | | X5282/X5289/X5293/X6184/X6185/X6344/X6346/X6347/X6348/X6349/X6350/ |
| | | X6351/X6352/X6353/X6355/X6356/X6357/X6358/X6359/X6363/X6364/ |
| | | X6365/X6366/X6367/X6368/X6369/X6461/X6462/X6470/X6478/X6490/X6491/ |
| | | X68/X7395/X7517/X7518/X7519/X7520/X7521/X7522/X7524/X7525/X7526/ |
| | | X7527/X7528/X7529/X7530/X7615/X7618/X7620/X7621/X7639/X7651/X7652/ |
| | | X854/X855/X858/X8633/X8634/X8635/X8636/X8707/X8709/X8712/X8714/ |
| | | X8715/X8716/X8742/X9672/X9674/X9677/X9689 |
| ASN_seq_aaAll_K | 1.28 | X117/X1188/X1190/X1192/X13/X1354/X1865/X1868/X1880/X263/X2741/X2753/ |
| | | X3794/X398/X45/X508/X509/X713/X716/X851/X852 |
| ASN_seq_aaAll_L | 1.28 | X1184/X1852/X2734 |
| ASN_seq_aaAll_M | 1.28 | X1206/X1207/X1208/X1266/X1267/X1883/X1884/X1952/X1953/X198/X25/X2756/ |
| | | X2827/X2828/X3865/X3947/X406/X407/X727/X728/X729/X78 |
| ASN_seq_aaAll_P | 1.28 | X1/X10426/X10428/X10494/X1162/X1163/X1164/X1165/X1166/X1167/X1168/ |
| | | X1169/X1170/X1171/X1172/X1175/X1176/X1177/X1179/X1180/X1181/X1182/ |
| | | X1183/X1184/X1185/X1186/X1219/X1222/X1223/X1224/X1225/X1226/ |
| | | X1228/X1229/X1230/X1231/X1232/X1233/X1234/X1235/X1236/X1237/X1238/ |
| | | X1239/X1240/X1356/X1357/X1358/X1359/X1360/X1361/X1362/X1363/X1364/ |
| | | X1365/X1431/X1432/X1433/X1435/X1437/X16/X17/X183/X1831/X1832/ |
| | | X1833/X1834/X1835/X1836/X1837/X1839/X184/X1840/X1841/X1842/X1843/ |
| | | X1844/X1847/X1848/X1849/X185/X1850/X1851/X1852/X1853/X1854/X1856/ |
| | | X1857/X1858/X1860/X1861/X1862/X189/X1898/X1899/X190/X1901/X1902/ |
| | | X1903/X1904/X1905/X1906/X1907/X1908/X1910/X1911/X1912/X1913/X1914/ |
| | | X1915/X1916/X1917/X1918/X1919/X1920/X1921/X1922/X1923/X1924/ |
| | | X1925/X1961/X2035/X2055/X2056/X2057/X2058/X2059/X2060/X2061/X2062/ |
| | | X2063/X2064/X2065/X2066/X2067/X2068/X2069/X2120/X214/X2145/X2146/ |
| | | X2148/X2149/X215/X2150/X2151/X2152/X2154/X2157/X216/X217/X218/ |
| | | X26/X265/X2699/X2700/X2701/X2702/X2703/X2704/X2705/X2706/X2707/ |
| | | X2708/X2709/X2712/X2713/X2715/X2716/X2717/X2718/X2723/X2724/X2725/ |
| | | X2727/X2728/X2729/X2732/X2733/X2734/X2735/X2736/X2738/X2739/X2740/ |
| | | X2768/X2769/X2770/X2771/X2773/X2774/X2775/X2776/X2777/X2779/ |
| | | X2780/X2781/X2782/X2783/X2785/X2786/X2789/X2790/X2791/X2792/X2793/ |
| | | X2794/X2795/X2796/X2797/X2798/X2799/X2855/X29/X2922/X2923/X2927/ |
| | | X2944/X2945/X2946/X2947/X2948/X2949/X2950/X2951/X2952/X2953/X2954/ |
| | | X2955/X2956/X2957/X2958/X2959/X2960/X2961/X2962/X2996/X2997/ |
| | | X3002/X3004/X3033/X3034/X3035/X3036/X3038/X3039/X3042/X3044/X3046/ |
| | | X3050/X3051/X3052/X3053/X3059/X3060/X3072/X32/X3742/X3743/X3744/ |
| | | X3745/X3746/X3747/X3749/X3750/X3752/X3753/X3754/X3755/X3760/X3761/ |
| | | X3762/X3763/X3766/X3767/X3768/X3769/X3773/X3774/X3775/X3776/ |
| | | X3777/X3779/X3780/X3781/X3782/X3783/X3784/X3787/X3805/X3813/X3814/ |
| | | X3815/X3816/X3817/X3819/X3820/X3823/X3825/X3826/X3827/X3828/X3829/ |
| | | X383/X3830/X3831/X3832/X3833/X3834/X3835/X3836/X385/X386/X387/ |
| | | X388/X389/X3907/X391/X394/X395/X396/X3966/X3970/X3972/X3974/X3976/ |
| | | X3991/X3992/X3993/X3994/X3995/X3996/X3997/X3998/X3999/X4000/ |
| | | X4001/X4002/X4003/X4004/X4005/X4006/X4007/X4008/X4009/X4025/X4026/ |
| | | X4032/X4033/X4037/X4038/X4039/X4040/X4042/X4066/X4067/X4070/X4072/ |
| | | X4073/X4074/X4079/X4080/X4083/X4087/X4088/X4089/X4090/X4095/ |
| | | X4097/X4098/X4099/X4102/X4103/X4105/X4109/X4123/X4130/X4132/X424/ |
| | | X425/X426/X427/X428/X429/X430/X4915/X4916/X4917/X4919/X4920/X4921/ |
| | | X4922/X4923/X4924/X4926/X4927/X4928/X4929/X4932/X4933/X4934/ |
| | | X4935/X4939/X4940/X4941/X4942/X4947/X4948/X4950/X4951/X4952/X4955/ |
| | | X4956/X4957/X4958/X4959/X4976/X4978/X4979/X4980/X4982/X4984/X4985/ |
| | | X4988/X4989/X4990/X4991/X510/X511/X512/X5120/X5123/X5125/X5127/ |
| | | X5129/X5134/X5141/X5142/X5143/X5144/X5145/X5146/X5147/X5148/X5149/ |
| | | X5150/X5151/X5152/X5153/X5154/X5155/X5161/X5164/X5165/X5169/ |
| | | X5170/X5171/X5172/X5173/X5174/X5178/X5179/X5180/X5181/X5200/X5201/ |
| | | X5211/X5212/X5213/X5220/X5221/X5227/X5229/X5230/X5231/X5233/X5237/ |
| | | X5238/X5239/X5242/X5249/X5252/X5253/X5276/X5278/X5282/X5287/ |
| | | X5289/X5293/X532/X6159/X6160/X6161/X6162/X6163/X6164/X6165/X6167/ |
| | | X6168/X6170/X6171/X6173/X6174/X6175/X6176/X6181/X6182/X6184/X6185/ |
| | | X6186/X6187/X6191/X6193/X6194/X6195/X6196/X6197/X6207/X6208/ |
| | | X6211/X6325/X6328/X6334/X6336/X6344/X6345/X6346/X6347/X6348/X6349/ |
| | | X6350/X6351/X6352/X6353/X6355/X6356/X6357/X6358/X6359/X6363/X6364/ |
| | | X6365/X6366/X6367/X6368/X6369/X6384/X6394/X6395/X6400/X6401/ |
| | | X6406/X6418/X6424/X6426/X6427/X6428/X6433/X6461/X6462/X6468/X6470/ |
| | | X6476/X6478/X6483/X6490/X6491/X67/X68/X694/X696/X697/X699/X7/X70/ |
| | | X700/X701/X702/X703/X705/X706/X707/X709/X71/X710/X711/X7379/X7380/ |
| | | X7381/X7382/X7384/X7385/X7387/X7388/X7389/X7390/X7392/X7394/ |
| | | X7395/X7396/X7397/X7401/X7403/X7404/X7406/X7407/X744/X746/X747/ |
| | | X748/X749/X750/X751/X7510/X7517/X7518/X7519/X752/X7520/X7521/X7522/ |
| | | X7524/X7525/X7526/X7527/X7528/X7529/X753/X7530/X754/X755/X7556/ |
| | | X756/X7563/X7564/X7575/X7615/X7617/X7618/X7620/X7621/X7628/X7637/ |
| | | X7639/X7651/X7652/X84/X854/X8542/X8543/X8545/X8546/X8547/X8548/ |
| | | X855/X8550/X8552/X8553/X8555/X8556/X8558/X856/X8560/X857/X858/ |
| | | X859/X8633/X8634/X8635/X8636/X8660/X8707/X8709/X8711/X8712/X8714/ |
| | | X8715/X8716/X8730/X8742/X90/X901/X903/X92/X93/X94/X9567/X9569/X9570/ |
| | | X9571/X9573/X9575/X9672/X9674/X9676/X9677/X9689 |
| ASN_seq_aaAll_Q | 1.28 | X1176/X1179/X1184/X1356/X1357/X1359/X1360/X1852/X187/X189/X19/X192/ |
| | | X2055/X2057/X2058/X2059/X2061/X2063/X21/X214/X2734/X2944/X2945/ |
| | | X2946/X2947/X2949/X2951/X2953/X2954/X2957/X388/X390/X392/X394/ |
| | | X3991/X3992/X3993/X3995/X3996/X3998/X3999/X4002/X4004/X4006/X510/ |
| | | X5141/X5142/X5144/X5145/X5146/X5147/X5149/X5151/X5154/X6344/X6345/ |
| | | X6346/X6348/X6349/X6352/X70/X700/X705/X708/X710/X73/X7517/X7519/ |
| | | X7520/X84/X855/X856/X8716 |
| ASN_seq_aaAll_R | 1.28 | X105/X1206/X1207/X1266/X1267/X1277/X1281/X1283/X1284/X1287/X1883/ |
| | | X1952/X1953/X1974/X1978/X198/X1980/X239/X241/X25/X2827/X2828/X2868/ |
| | | X3865/X406/X407/X461/X462/X464/X727/X728/X729/X78/X787/X789/ |
| | | X792/X793/X795/X83 |
| ASN_seq_aaAll_S | 1.28 | X1167/X1174/X1177/X1206/X1207/X1208/X16/X183/X1835/X1848/X1849/X1883/ |
| | | X1884/X190/X198/X25/X2723/X2725/X2756/X386/X389/X395/X406/X407/ |
| | | X6/X67/X698/X704/X706/X71/X711/X727/X728/X729/X78 |
| ASN_seq_aaAll_T | 1.28 | X117/X13/X1354/X263/X45/X508/X509/X851/X852 |
| ASN_seq_aaAll_V | 1.28 | X1881/X24/X2754/X3795/X77 |
| ASN_seq_aaAll_W | 1.28 | X10469/X10472/X10473/X10483/X10495/X11117/X1166/X1174/X1188/X1190/ |
| | | X1191/X1192/X1357/X1360/X1362/X1363/X1364/X1439/X1440/X16/X183/ |
| | | X1847/X1849/X1865/X1868/X1869/X1894/X193/X194/X195/X196/X2058/X2059/ |
| | | X2060/X2061/X2064/X2065/X2068/X214/X2161/X2162/X2163/X2166/ |
| | | X23/X254/X265/X2724/X2741/X2763/X29/X2945/X2947/X2948/X2949/X2954/ |
| | | X2959/X2961/X3064/X3065/X3066/X3070/X3072/X3073/X3074/X3775/X3802/ |
| | | X386/X397/X398/X399/X3991/X3999/X4003/X4004/X4007/X401/X402/ |
| | | X4114/X4116/X4117/X4119/X4123/X4124/X4125/X4127/X4129/X4130/X4133/ |
| | | X4134/X489/X510/X512/X5146/X5148/X5149/X5262/X5264/X5265/X5267/ |
| | | X5268/X5270/X5273/X5275/X5276/X5279/X5280/X5282/X5283/X5284/X5285/ |
| | | X5286/X5287/X5290/X6345/X6445/X6447/X6448/X6450/X6452/X6453/X6456/ |
| | | X6457/X6459/X6461/X6463/X6465/X6466/X6467/X6468/X6471/X6475/ |
| | | X6476/X6479/X6480/X6481/X6482/X6483/X67/X698/X704/X713/X714/X715/ |
| | | X716/X717/X75/X7593/X7594/X7597/X7598/X76/X7600/X7602/X7603/X7605/ |
| | | X7606/X7609/X7610/X7616/X7617/X7623/X7625/X7626/X7627/X7628/ |
| | | X7635/X7636/X7637/X7640/X7643/X84/X856/X857/X859/X8680/X8681/X8683/ |
| | | X8684/X8687/X8688/X8692/X8695/X8696/X8698/X8699/X8708/X8710/X8711/ |
| | | X8717/X8720/X8728/X8729/X8730/X90/X907/X9643/X9646/X9647/X9649/ |
| | | X9650/X9658/X9661/X9662/X9673/X9675/X9676/X9690 |
| ASN_seq_aaAll_Y | 1.28 | X104/X1367/X1371/X2073/X234/X235/X249/X39/X452/X453/X513/X799/X860/X863 |
| ASN_seq_aaDown_A | 1.28 | X1/X10425/X11095/X1162/X1163/X1164/X1169/X117/X1171/X1172/X1177/ |
| | | X1219/X1221/X1222/X1223/X1224/X1225/X1226/X1228/X1229/X1230/X1231/ |
| | | X1232/X1233/X1234/X1235/X1236/X1237/X1238/X1239/X1240/X13/X1354/ |
| | | X1355/X1431/X1432/X1433/X1435/X1437/X1464/X1479/X1831/X1832/X1834/ |
| | | X1836/X1837/X184/X1840/X1842/X1844/X1895/X1898/X1899/X1901/X1902/ |
| | | X1903/X1904/X1905/X1906/X1907/X1908/X1910/X1911/X1912/X1913/ |
| | | X1914/X1915/X1916/X1917/X1918/X1919/X1920/X1921/X1922/X1923/X1924/ |
| | | X1925/X2035/X2036/X2054/X2145/X2146/X2148/X2149/X215/X2150/X2151/ |
| | | X2152/X2154/X2157/X216/X217/X218/X2186/X2211/X2212/X26/X263/ |
| | | X2699/X2700/X2702/X2705/X2707/X2709/X2712/X2713/X2716/X2718/X2766/ |
| | | X2768/X2769/X2770/X2771/X2773/X2774/X2775/X2776/X2777/X2779/X2780/ |
| | | X2781/X2782/X2783/X2785/X2786/X2788/X2789/X2790/X2791/X2792/ |
| | | X2793/X2794/X2795/X2796/X2797/X2798/X2799/X2922/X2923/X2924/X2926/ |
| | | X2927/X2928/X3032/X3033/X3034/X3035/X3036/X3038/X3039/X3040/X3042/ |
| | | X3044/X3045/X3046/X3050/X3051/X3052/X3053/X3059/X3060/X3062/ |
| | | X3115/X3116/X3117/X32/X3742/X3744/X3746/X3747/X3749/X3750/X3753/ |
| | | X3755/X3761/X3763/X3805/X3811/X3813/X3814/X3815/X3816/X3817/X3819/ |
| | | X3820/X3822/X3823/X3825/X3826/X3827/X3828/X3829/X383/X3830/X3831/ |
| | | X3832/X3833/X3834/X3835/X3836/X385/X3966/X3968/X3970/X3972/X3973/ |
| | | X3974/X3976/X3977/X3978/X4066/X4067/X4069/X4070/X4071/X4072/ |
| | | X4073/X4074/X4076/X4078/X4079/X4080/X4081/X4082/X4083/X4087/X4088/ |
| | | X4089/X4090/X4093/X4095/X4097/X4098/X4099/X4102/X4103/X4105/X4108/ |
| | | X4109/X4111/X4112/X4166/X424/X425/X426/X427/X428/X429/X430/X45/ |
| | | X4916/X4917/X4919/X4923/X4924/X4927/X4940/X4942/X4974/X4976/X4978/ |
| | | X4979/X4980/X4981/X4982/X4984/X4985/X4987/X4988/X4989/X4990/ |
| | | X4991/X508/X509/X5120/X5123/X5125/X5127/X5128/X5129/X5131/X5133/ |
| | | X5134/X5135/X5200/X5201/X5203/X5204/X5205/X5206/X5208/X5210/X5211/ |
| | | X5212/X5213/X5214/X5217/X5220/X5221/X5222/X5225/X5227/X5229/X5230/ |
| | | X5231/X5232/X5233/X5237/X5238/X5239/X5242/X5243/X5244/X5245/ |
| | | X5248/X5249/X5252/X5253/X5254/X5255/X5259/X532/X6/X6159/X6164/X6165/ |
| | | X6167/X6168/X6171/X6201/X6205/X6207/X6208/X6211/X6325/X6328/ |
| | | X6331/X6334/X6335/X6336/X6338/X6339/X6381/X6383/X6384/X6387/X6391/ |
| | | X6394/X6395/X6397/X6399/X6400/X6401/X6406/X6407/X6409/X6413/X6414/ |
| | | X6415/X6418/X6419/X6420/X6422/X6424/X6426/X6427/X6428/X6430/ |
| | | X6431/X6432/X6433/X6438/X68/X696/X697/X7/X703/X7379/X7380/X7382/ |
| | | X7385/X7389/X7390/X744/X746/X747/X748/X749/X750/X751/X7510/X7513/ |
| | | X7514/X7515/X752/X753/X754/X7541/X755/X7550/X7551/X7556/X7557/ |
| | | X756/X7560/X7563/X7564/X7568/X7570/X7571/X7572/X7575/X7576/X7577/ |
| | | X7578/X851/X852/X853/X8542/X8543/X8553/X8631/X8654/X8655/X8660/ |
| | | X8661/X8664/X901/X903/X92/X93/X94/X9566/X9569/X9630 |
| ASN_seq_aaDown_C | 1.28 | X105/X117/X1179/X1274/X1283/X1284/X1289/X13/X1354/X1355/X1464/X1479/ |
| | | X187/X1880/X192/X1971/X1976/X1980/X1984/X2054/X21/X2186/X2212/ |
| | | X241/X263/X2753/X2857/X2861/X2870/X3116/X3117/X3794/X390/X392/ |
| | | X3922/X45/X461/X464/X508/X509/X700/X708/X73/X789/X792/X795/X851/X852/X853 |
| ASN_seq_aaDown_D | 1.28 | X1162/X1163/X1165/X1167/X1170/X1171/X1175/X1177/X1180/X1183/X1185/ |
| | | X1186/X1223/X1224/X1225/X1226/X1231/X1240/X1266/X1267/X1831/X1833/ |
| | | X1834/X1836/X1841/X1842/X1844/X1848/X185/X1851/X1853/X1854/X1856/ |
| | | X1858/X1861/X1862/X190/X1904/X1907/X1908/X1914/X1919/X1952/ |
| | | X1953/X2035/X216/X217/X26/X2699/X2701/X2702/X2706/X2707/X2709/X2712/ |
| | | X2715/X2716/X2717/X2718/X2723/X2725/X2728/X2729/X2733/X2735/ |
| | | X2736/X2739/X2740/X2777/X2782/X2799/X2827/X2828/X2923/X3742/X3744/ |
| | | X3746/X3747/X3752/X3753/X3754/X3755/X3760/X3761/X3763/X3774/X3777/ |
| | | X3780/X3781/X3783/X3784/X3787/X3829/X383/X3865/X389/X391/X395/ |
| | | X3970/X424/X426/X427/X4916/X4917/X4919/X4921/X4939/X4940/X4941/ |
| | | X4942/X4952/X4955/X4957/X4958/X4959/X6/X6162/X6167/X6168/X6196/ |
| | | X6197/X694/X697/X699/X7/X701/X706/X709/X71/X711/X7387/X7407/X744/ |
| | | X748/X749/X751/X752/X92 |
| ASN_seq_aaDown_E | 1.28 | X393 |
| ASN_seq_aaDown_F | 1.28 | X110/X1189/X1194/X1329/X1370/X1867/X2072/X255/X2743/X400/X491/X492/ |
| | | X718/X830/X831/X862 |
| ASN_seq_aaDown_G | 1.28 | X104/X107/X110/X1184/X1329/X1357/X1360/X1362/X1363/X1364/X1367/X1370/ |
| | | X1371/X1372/X1417/X1440/X1832/X1852/X2058/X2059/X2060/X2061/ |
| | | X2064/X2065/X2068/X2072/X2073/X2124/X214/X2162/X2166/X234/X235/ |
| | | X249/X254/X255/X265/X267/X2734/X2738/X29/X2945/X2947/X2948/X2949/ |
| | | X2954/X2959/X2961/X3007/X3065/X3070/X3073/X3750/X3775/X3779/X39/ |
| | | X3991/X3999/X4003/X4004/X4007/X4114/X4119/X4124/X4127/X4130/X452/ |
| | | X453/X489/X491/X492/X4928/X4959/X510/X512/X513/X514/X5146/X5148/ |
| | | X5149/X515/X5264/X5273/X5276/X5283/X6193/X6196/X6345/X6452/X6463/ |
| | | X6476/X7407/X7593/X7617/X799/X830/X831/X84/X856/X8560/X857/X859/ |
| | | X860/X862/X863/X864/X865/X90/X907/X9575 |
| ASN_seq_aaDown_H | 1.28 | X1/X10459/X1162/X1163/X1164/X1165/X1167/X1169/X1170/X1171/X1172/ |
| | | X1177/X1180/X1181/X1186/X1219/X1220/X1221/X1222/X1227/X1228/X1229/ |
| | | X1230/X1232/X1233/X1234/X1235/X1236/X1237/X1238/X1239/X1317/X1320/ |
| | | X1321/X1431/X1432/X1433/X1435/X1437/X1464/X1479/X1557/X17/X1831/ |
| | | X1832/X1833/X1834/X1836/X1837/X184/X1840/X1841/X1842/X1844/X1848/ |
| | | X185/X1854/X1856/X1858/X187/X1895/X1896/X1897/X1898/X1899/X190/ |
| | | X1900/X1901/X1902/X1903/X1905/X1906/X1909/X1910/X1911/X1912/ |
| | | X1913/X1914/X1915/X1916/X1917/X1918/X192/X1920/X1921/X1922/X1923/ |
| | | X1924/X1925/X2012/X2021/X2023/X2035/X2036/X21/X2145/X2146/X2148/ |
| | | X2149/X215/X2150/X2151/X2152/X2154/X2157/X2160/X218/X2186/X2211/ |
| | | X2212/X2331/X2332/X253/X2699/X2700/X2701/X2702/X2705/X2706/X2707/ |
| | | X2709/X2712/X2713/X2715/X2716/X2718/X2723/X2725/X2733/X2736/X2764/ |
| | | X2765/X2766/X2767/X2768/X2769/X2770/X2771/X2772/X2773/X2774/ |
| | | X2775/X2776/X2777/X2778/X2779/X2780/X2781/X2783/X2784/X2785/X2786/ |
| | | X2787/X2788/X2789/X2790/X2791/X2792/X2793/X2794/X2795/X2796/X2797/ |
| | | X2798/X2910/X2913/X2922/X2923/X2924/X2925/X2926/X2927/X2928/ |
| | | X3032/X3033/X3034/X3035/X3036/X3038/X3039/X3040/X3042/X3044/X3045/ |
| | | X3046/X3050/X3051/X3052/X3053/X3056/X3059/X3060/X3061/X3062/X3115/ |
| | | X3116/X3117/X32/X3285/X3286/X3287/X3293/X3742/X3743/X3744/X3746/ |
| | | X3747/X3749/X3750/X3752/X3753/X3755/X3760/X3761/X3763/X3768/ |
| | | X3774/X3803/X3804/X3805/X3806/X3807/X3808/X3810/X3811/X3812/X3813/ |
| | | X3814/X3815/X3816/X3817/X3818/X3819/X3820/X3821/X3822/X3823/X3824/ |
| | | X3825/X3826/X3827/X3828/X3829/X383/X3830/X3831/X3832/X3833/X3834/ |
| | | X3835/X3836/X385/X389/X390/X391/X392/X395/X3958/X396/X3966/ |
| | | X3967/X3968/X3969/X3970/X3972/X3973/X3974/X3975/X3976/X3977/X3978/ |
| | | X4066/X4067/X4069/X4070/X4071/X4072/X4073/X4074/X4075/X4076/X4078/ |
| | | X4079/X4080/X4081/X4082/X4083/X4087/X4088/X4089/X4090/X4093/ |
| | | X4095/X4096/X4097/X4098/X4099/X4102/X4103/X4104/X4105/X4108/X4109/ |
| | | X4111/X4112/X4113/X4166/X425/X428/X429/X430/X4373/X4374/X4376/ |
| | | X4378/X4384/X4389/X4392/X4394/X487/X4915/X4916/X4917/X4919/X4921/ |
| | | X4923/X4924/X4926/X4927/X4929/X4934/X4939/X4940/X4942/X4969/X4970/ |
| | | X4971/X4973/X4974/X4975/X4976/X4977/X4978/X4979/X4980/X4981/X4982/ |
| | | X4983/X4984/X4985/X4986/X4987/X4988/X4989/X4990/X4991/X5119/ |
| | | X5120/X5123/X5124/X5125/X5127/X5128/X5129/X5130/X5131/X5132/X5133/ |
| | | X5134/X5135/X5200/X5201/X5202/X5203/X5204/X5205/X5206/X5207/X5208/ |
| | | X5210/X5211/X5212/X5213/X5214/X5215/X5217/X5218/X5219/X5220/ |
| | | X5221/X5222/X5225/X5227/X5228/X5229/X5230/X5231/X5232/X5233/X5237/ |
| | | X5238/X5239/X5240/X5241/X5242/X5243/X5244/X5245/X5248/X5249/X5252/ |
| | | X5253/X5254/X5255/X5256/X5257/X5258/X5259/X5260/X5309/X5310/ |
| | | X532/X5564/X5565/X5566/X5570/X5575/X5580/X5583/X5586/X5588/X5590/ |
| | | X5594/X6/X6160/X6162/X6164/X6165/X6167/X6168/X6170/X6171/X6173/ |
| | | X6174/X6176/X6186/X6200/X6201/X6202/X6204/X6205/X6206/X6207/X6208/ |
| | | X6210/X6211/X6325/X6328/X6329/X6330/X6331/X6332/X6334/X6335/X6336/ |
| | | X6337/X6338/X6339/X6381/X6382/X6383/X6384/X6385/X6386/X6387/ |
| | | X6388/X6389/X6391/X6392/X6393/X6394/X6395/X6396/X6397/X6399/X6400/ |
| | | X6401/X6403/X6404/X6405/X6406/X6407/X6408/X6409/X6413/X6414/X6415/ |
| | | X6416/X6417/X6418/X6419/X6420/X6422/X6424/X6425/X6426/X6427/ |
| | | X6428/X6430/X6431/X6432/X6433/X6434/X6435/X6436/X6437/X6438/X6439/ |
| | | X6440/X6441/X6442/X6443/X6503/X6506/X6507/X6760/X6762/X6764/X6767/ |
| | | X6772/X6773/X6777/X6787/X6793/X6796/X6798/X68/X6812/X694/X696/ |
| | | X697/X699/X701/X703/X706/X707/X71/X711/X73/X7380/X7381/X7382/ |
| | | X7384/X7385/X7387/X7389/X7390/X7409/X7412/X745/X746/X747/X750/X7507/ |
| | | X7510/X7511/X7513/X7514/X7515/X7516/X753/X7537/X7538/X7539/X754/ |
| | | X7540/X7541/X7544/X7545/X7547/X7549/X755/X7550/X7551/X7553/X7554/ |
| | | X7555/X7556/X7557/X7558/X756/X7560/X7561/X7562/X7563/X7564/ |
| | | X7566/X7568/X7570/X7571/X7572/X7573/X7574/X7575/X7576/X7577/X7578/ |
| | | X7579/X7580/X7581/X7582/X7583/X7584/X7585/X7586/X7587/X7589/X7662/ |
| | | X7664/X7665/X7912/X7914/X7916/X7922/X7926/X7931/X7946/X7948/ |
| | | X7961/X823/X824/X8542/X8545/X8547/X8552/X8553/X8631/X8632/X8642/ |
| | | X8645/X8646/X8647/X8648/X8651/X8654/X8655/X8657/X8658/X8659/X8660/ |
| | | X8661/X8664/X8665/X8666/X8667/X8668/X8669/X8670/X8673/X8674/X8977/ |
| | | X8979/X8990/X9000/X901/X903/X9031/X9041/X9043/X93/X94/X9569/ |
| | | X9624/X9625/X9630/X9631/X9632/X9634/X9635/X9903/X9932 |
| ASN_seq_aaDown_I | 1.28 | X1/X1176/X1184/X1357/X1358/X1360/X1361/X1362/X1363/X1364/X1365/X17/ |
| | | X1852/X1857/X19/X2056/X2058/X2059/X2060/X2061/X2062/X2064/X2065/ |
| | | X2066/X2067/X2068/X2069/X214/X265/X2734/X29/X2945/X2947/X2948/ |
| | | X2949/X2950/X2952/X2954/X2955/X2956/X2958/X2959/X2960/X2961/X2962/ |
| | | X3/X3991/X3994/X3997/X3999/X4000/X4001/X4003/X4004/X4005/X4007/ |
| | | X4008/X4009/X510/X511/X512/X5143/X5146/X5148/X5149/X5150/X5152/ |
| | | X5153/X5155/X6191/X6345/X6347/X6350/X6351/X68/X710/X7395/X7401/ |
| | | X7518/X84/X854/X8558/X856/X857/X858/X859/X90/X9575 |
| ASN_seq_aaDown_L | 1.28 | X1/X1206/X1207/X1266/X1883/X1952/X198/X25/X2827/X406/X407/X6/X727/ |
| | | X728/X729/X78 |
| ASN_seq_aaDown_M | 1.28 | X104/X110/X1206/X1207/X1328/X1329/X1330/X1883/X198/X2083/X234/X235/ |
| | | X25/X255/X39/X3947/X406/X407/X452/X453/X491/X492/X493/X727/X728/ |
| | | X729/X78/X799/X829/X830/X831/X832 |
| ASN_seq_aaDown_N | 1.28 | X1/X10459/X1162/X1163/X1164/X1165/X1167/X1169/X1170/X1171/X1172/ |
| | | X1175/X1176/X1177/X1179/X1180/X1181/X1183/X1185/X1219/X1220/X1221/ |
| | | X1222/X1227/X1228/X1229/X1230/X1232/X1233/X1234/X1235/X1236/X1237/ |
| | | X1238/X1239/X1431/X1432/X1433/X1435/X1437/X1557/X17/X1831/X1832/ |
| | | X1833/X1834/X1835/X1836/X1837/X184/X1840/X1841/X1842/X1843/X1844/ |
| | | X1848/X185/X1851/X1853/X1856/X1857/X1858/X187/X189/X1895/X1896/ |
| | | X1897/X1898/X1899/X19/X190/X1900/X1901/X1902/X1903/X1905/X1906/ |
| | | X1909/X1910/X1911/X1912/X1913/X1914/X1915/X1916/X1917/X1918/X192/ |
| | | X1920/X1921/X1922/X1923/X1924/X1925/X2035/X2036/X21/X2145/X2146/ |
| | | X2148/X2149/X215/X2150/X2151/X2152/X2154/X2157/X2160/X218/X2331/ |
| | | X2332/X2699/X2700/X2701/X2702/X2703/X2705/X2706/X2707/X2708/ |
| | | X2709/X2712/X2713/X2715/X2716/X2717/X2718/X2723/X2724/X2725/X2733/ |
| | | X2735/X2736/X2764/X2765/X2766/X2767/X2768/X2769/X2770/X2771/X2772/ |
| | | X2773/X2774/X2775/X2776/X2777/X2778/X2779/X2780/X2781/X2783/ |
| | | X2784/X2785/X2786/X2787/X2788/X2789/X2790/X2791/X2792/X2793/X2794/ |
| | | X2795/X2796/X2797/X2798/X2922/X2923/X2924/X2925/X2926/X2927/X2928/ |
| | | X3/X3032/X3033/X3034/X3035/X3036/X3038/X3039/X3040/X3042/X3044/ |
| | | X3045/X3046/X3050/X3051/X3052/X3053/X3056/X3059/X3060/X3061/X3062/ |
| | | X32/X3285/X3286/X3287/X3293/X3742/X3744/X3745/X3746/X3747/X3749/ |
| | | X3750/X3752/X3753/X3754/X3755/X3760/X3761/X3762/X3763/X3766/ |
| | | X3768/X3774/X3776/X3803/X3804/X3805/X3806/X3807/X3808/X3810/X3811/ |
| | | X3812/X3813/X3814/X3815/X3816/X3817/X3818/X3819/X3820/X3821/X3822/ |
| | | X3823/X3824/X3825/X3826/X3827/X3828/X3829/X383/X3830/X3831/ |
| | | X3832/X3833/X3834/X3835/X3836/X385/X388/X389/X390/X391/X392/X394/ |
| | | X395/X396/X3966/X3967/X3968/X3969/X3970/X3972/X3973/X3974/X3975/ |
| | | X3976/X3977/X3978/X4066/X4067/X4069/X4070/X4071/X4072/X4073/X4074/ |
| | | X4075/X4076/X4078/X4079/X4080/X4081/X4082/X4083/X4087/X4088/X4089/ |
| | | X4090/X4093/X4095/X4096/X4097/X4098/X4099/X4102/X4103/X4104/ |
| | | X4105/X4108/X4109/X4111/X4112/X4113/X425/X428/X429/X430/X4373/X4374/ |
| | | X4376/X4378/X4384/X4389/X4392/X4394/X4915/X4916/X4917/X4919/ |
| | | X4920/X4921/X4922/X4923/X4924/X4927/X4929/X4932/X4934/X4939/X4940/ |
| | | X4941/X4942/X4969/X4970/X4971/X4973/X4974/X4975/X4976/X4977/X4978/ |
| | | X4979/X4980/X4981/X4982/X4983/X4984/X4985/X4986/X4987/X4988/ |
| | | X4989/X4990/X4991/X5119/X5120/X5123/X5124/X5125/X5127/X5128/X5129/ |
| | | X5130/X5131/X5132/X5133/X5134/X5135/X5200/X5201/X5202/X5203/X5204/ |
| | | X5205/X5206/X5207/X5208/X5210/X5211/X5212/X5213/X5214/X5215/ |
| | | X5217/X5218/X5219/X5220/X5221/X5222/X5225/X5227/X5228/X5229/X5230/ |
| | | X5231/X5232/X5233/X5237/X5238/X5239/X5240/X5241/X5242/X5243/X5244/ |
| | | X5245/X5248/X5249/X5252/X5253/X5254/X5255/X5256/X5257/X5258/ |
| | | X5259/X5260/X5309/X5310/X532/X5564/X5565/X5566/X5570/X5575/X5580/ |
| | | X5583/X5586/X5588/X5590/X5594/X6159/X6160/X6162/X6163/X6164/X6165/ |
| | | X6167/X6168/X6171/X6174/X6184/X6186/X6200/X6201/X6202/X6204/ |
| | | X6205/X6206/X6207/X6208/X6210/X6211/X6325/X6328/X6329/X6330/X6331/ |
| | | X6332/X6334/X6335/X6336/X6337/X6338/X6339/X6381/X6382/X6383/X6384/ |
| | | X6385/X6386/X6387/X6388/X6389/X6391/X6392/X6393/X6394/X6395/ |
| | | X6396/X6397/X6399/X6400/X6401/X6403/X6404/X6405/X6406/X6407/X6408/ |
| | | X6409/X6413/X6414/X6415/X6416/X6417/X6418/X6419/X6420/X6422/X6424/ |
| | | X6425/X6426/X6427/X6428/X6430/X6431/X6432/X6433/X6434/X6435/ |
| | | X6436/X6437/X6438/X6439/X6440/X6441/X6442/X6443/X6503/X6506/X6507/ |
| | | X6760/X6762/X6764/X6767/X6772/X6773/X6777/X6787/X6793/X6796/X6798/ |
| | | X68/X6812/X694/X696/X697/X699/X70/X700/X701/X703/X705/X706/X707/ |
| | | X708/X709/X71/X710/X711/X73/X7379/X7380/X7381/X7382/X7385/X7387/ |
| | | X7388/X7389/X7390/X7409/X7412/X745/X746/X747/X750/X7507/X7510/ |
| | | X7511/X7513/X7514/X7515/X7516/X753/X7537/X7538/X7539/X754/X7540/ |
| | | X7541/X7544/X7545/X7547/X7549/X755/X7550/X7551/X7553/X7554/X7555/ |
| | | X7556/X7557/X7558/X756/X7560/X7561/X7562/X7563/X7564/X7566/X7568/ |
| | | X7570/X7571/X7572/X7573/X7574/X7575/X7576/X7577/X7578/X7579/ |
| | | X7580/X7581/X7582/X7583/X7584/X7585/X7586/X7587/X7589/X7662/X7664/ |
| | | X7665/X7912/X7914/X7916/X7922/X7926/X7931/X7946/X7948/X7961/X8542/ |
| | | X8543/X8547/X8553/X8631/X8632/X8642/X8645/X8646/X8647/X8648/ |
| | | X8651/X8654/X8655/X8657/X8658/X8659/X8660/X8661/X8664/X8665/X8666/ |
| | | X8667/X8668/X8669/X8670/X8673/X8674/X8977/X8979/X8990/X9000/X901/ |
| | | X903/X9031/X9041/X9043/X93/X94/X9567/X9569/X9624/X9625/X9630/ |
| | | X9631/X9632/X9634/X9635/X9903/X9932 |
| ASN_seq_aaDown_P | 1.28 | X1/X10425/X10426/X10427/X10428/X10429/X10459/X11095/X11096/X11581/ |
| | | X1162/X1163/X1164/X1165/X1166/X1167/X1168/X1169/X1170/X1171/X1172/ |
| | | X1174/X1175/X1176/X1177/X1179/X1180/X1181/X1182/X1183/X1184/ |
| | | X1185/X1186/X1219/X1220/X1221/X1222/X1223/X1224/X1225/X1226/X1227/ |
| | | X1228/X1229/X1230/X1231/X1232/X1233/X1234/X1235/X1236/X1237/X1238/ |
| | | X1239/X1240/X1431/X1432/X1433/X1435/X1437/X17/X1831/X1832/X1833/ |
| | | X1834/X1835/X1836/X1837/X1839/X184/X1840/X1841/X1842/X1843/X1844/ |
| | | X1847/X1848/X1849/X185/X1850/X1851/X1852/X1853/X1854/X1856/ |
| | | X1857/X1858/X1860/X1861/X1862/X189/X1895/X1896/X1897/X1898/X1899/ |
| | | X19/X190/X1900/X1901/X1902/X1903/X1904/X1905/X1906/X1907/X1908/ |
| | | X1909/X1910/X1911/X1912/X1913/X1914/X1915/X1916/X1917/X1918/X1919/ |
| | | X1920/X1921/X1922/X1923/X1924/X1925/X2035/X2036/X2145/X2146/X2148/ |
| | | X2149/X215/X2150/X2151/X2152/X2154/X2157/X216/X2160/X217/X218/ |
| | | X26/X2699/X2700/X2701/X2702/X2703/X2704/X2705/X2706/X2707/X2708/ |
| | | X2709/X2712/X2713/X2715/X2716/X2717/X2718/X2723/X2724/X2725/X2727/ |
| | | X2728/X2729/X2732/X2733/X2734/X2735/X2736/X2738/X2739/X2740/ |
| | | X2764/X2765/X2766/X2767/X2768/X2769/X2770/X2771/X2772/X2773/X2774/ |
| | | X2775/X2776/X2777/X2778/X2779/X2780/X2781/X2782/X2783/X2784/X2785/ |
| | | X2786/X2788/X2789/X2790/X2791/X2792/X2793/X2794/X2795/X2796/ |
| | | X2797/X2798/X2799/X2922/X2923/X2924/X2925/X2926/X2927/X2928/X3/X3032/ |
| | | X3033/X3034/X3035/X3036/X3038/X3039/X3040/X3042/X3044/X3045/ |
| | | X3046/X3050/X3051/X3052/X3053/X3056/X3059/X3060/X3061/X3062/X32/ |
| | | X3742/X3743/X3744/X3745/X3746/X3747/X3749/X3750/X3752/X3753/X3754/ |
| | | X3755/X3760/X3761/X3762/X3763/X3766/X3767/X3768/X3769/X3773/X3774/ |
| | | X3775/X3776/X3777/X3779/X3780/X3781/X3782/X3783/X3784/X3787/ |
| | | X3803/X3804/X3805/X3806/X3807/X3808/X3811/X3813/X3814/X3815/X3816/ |
| | | X3817/X3818/X3819/X3820/X3822/X3823/X3824/X3825/X3826/X3827/X3828/ |
| | | X3829/X383/X3830/X3831/X3832/X3833/X3834/X3835/X3836/X385/X387/ |
| | | X388/X389/X391/X394/X395/X396/X3966/X3967/X3968/X3969/X3970/ |
| | | X3972/X3973/X3974/X3975/X3976/X3977/X3978/X4066/X4067/X4069/X4070/ |
| | | X4071/X4072/X4073/X4074/X4075/X4076/X4078/X4079/X4080/X4081/X4082/ |
| | | X4083/X4087/X4088/X4089/X4090/X4093/X4095/X4097/X4098/X4099/ |
| | | X4102/X4103/X4104/X4105/X4108/X4109/X4111/X4112/X4113/X424/X425/ |
| | | X426/X427/X428/X429/X430/X4915/X4916/X4917/X4919/X4920/X4921/X4922/ |
| | | X4923/X4924/X4926/X4927/X4928/X4929/X4932/X4933/X4934/X4935/X4939/ |
| | | X4940/X4941/X4942/X4947/X4948/X4950/X4951/X4952/X4955/X4956/ |
| | | X4957/X4958/X4959/X4971/X4974/X4975/X4976/X4978/X4979/X4980/X4981/ |
| | | X4982/X4983/X4984/X4985/X4987/X4988/X4989/X4990/X4991/X5119/X5120/ |
| | | X5123/X5124/X5125/X5127/X5128/X5129/X5130/X5131/X5132/X5133/ |
| | | X5134/X5135/X5200/X5201/X5202/X5203/X5204/X5205/X5206/X5207/X5208/ |
| | | X5210/X5211/X5212/X5213/X5214/X5215/X5217/X5220/X5221/X5222/X5225/ |
| | | X5227/X5229/X5230/X5231/X5232/X5233/X5237/X5238/X5239/X5240/ |
| | | X5242/X5243/X5244/X5245/X5248/X5249/X5252/X5253/X5254/X5255/X5256/ |
| | | X5257/X5258/X5259/X5260/X532/X6159/X6160/X6161/X6162/X6163/X6164/ |
| | | X6165/X6167/X6168/X6170/X6171/X6173/X6174/X6175/X6176/X6181/ |
| | | X6182/X6184/X6185/X6186/X6187/X6191/X6193/X6194/X6195/X6196/X6197/ |
| | | X6201/X6202/X6205/X6207/X6208/X6210/X6211/X6325/X6328/X6329/X6330/ |
| | | X6331/X6332/X6334/X6335/X6336/X6337/X6338/X6339/X6381/X6382/ |
| | | X6383/X6384/X6385/X6387/X6389/X6391/X6394/X6395/X6396/X6397/X6399/ |
| | | X6400/X6401/X6403/X6406/X6407/X6408/X6409/X6413/X6414/X6415/X6416/ |
| | | X6418/X6419/X6420/X6422/X6424/X6426/X6427/X6428/X6430/X6431/ |
| | | X6432/X6433/X6435/X6436/X6437/X6438/X6439/X6440/X6442/X6443/X6503/ |
| | | X68/X694/X696/X697/X698/X699/X7/X70/X700/X701/X702/X703/X705/X706/ |
| | | X707/X709/X71/X710/X711/X7379/X7380/X7381/X7382/X7384/X7385/ |
| | | X7387/X7388/X7389/X7390/X7392/X7393/X7394/X7395/X7396/X7397/X7401/ |
| | | X7403/X7404/X7406/X7407/X7412/X744/X745/X746/X747/X748/X749/X750/ |
| | | X7507/X751/X7510/X7511/X7513/X7514/X7515/X7516/X752/X753/X7537/ |
| | | X7538/X754/X7540/X7541/X7544/X7547/X755/X7550/X7551/X7553/X7556/ |
| | | X7557/X7558/X756/X7560/X7563/X7564/X7568/X7570/X7571/X7572/X7573/ |
| | | X7575/X7576/X7577/X7578/X7579/X7580/X7581/X7582/X7584/X7585/X7586/ |
| | | X7587/X7589/X7662/X8542/X8543/X8544/X8545/X8546/X8547/X8548/ |
| | | X8550/X8551/X8552/X8553/X8555/X8556/X8558/X8560/X8631/X8632/X8645/ |
| | | X8647/X8654/X8655/X8657/X8660/X8661/X8664/X8665/X8666/X8667/X8668/ |
| | | X8669/X8670/X8673/X8674/X901/X903/X9031/X92/X93/X94/X9566/X9567/ |
| | | X9568/X9569/X9570/X9571/X9573/X9574/X9575/X9624/X9630/X9631/ |
| | | X9632/X9634/X9635 |
| ASN_seq_aaDown_Q | 1.28 | X10469/X10472/X10473/X10483/X10495/X11117/X1168/X1175/X1176/X1179/ |
| | | X1182/X1183/X1184/X1358/X1361/X1362/X1363/X1364/X1365/X1439/X1440/ |
| | | X1839/X185/X1850/X1851/X1852/X1856/X1857/X1860/X1861/X187/X189/ |
| | | X19/X192/X2056/X2060/X2062/X2064/X2065/X2066/X2067/X2068/X2069/ |
| | | X21/X2161/X2162/X2163/X2166/X254/X265/X2727/X2728/X2732/X2734/ |
| | | X2738/X2739/X2740/X29/X2948/X2950/X2952/X2955/X2956/X2958/X2959/ |
| | | X2960/X2961/X2962/X3064/X3065/X3066/X3070/X3073/X3074/X3773/X3775/ |
| | | X3779/X3780/X3781/X3782/X3783/X3787/X387/X388/X391/X392/X394/X3994/ |
| | | X3997/X4000/X4001/X4003/X4005/X4007/X4008/X4009/X4114/X4116/ |
| | | X4117/X4119/X4124/X4125/X4127/X4129/X4130/X4133/X4134/X489/X4955/ |
| | | X4956/X4957/X4958/X4959/X511/X512/X5143/X5148/X5150/X5152/X5153/ |
| | | X5155/X5262/X5264/X5265/X5267/X5268/X5270/X5273/X5275/X5276/X5279/ |
| | | X5280/X5283/X5284/X5285/X5286/X5287/X5290/X6193/X6196/X6197/ |
| | | X6347/X6350/X6351/X6445/X6447/X6448/X6450/X6452/X6453/X6456/X6457/ |
| | | X6459/X6463/X6465/X6466/X6467/X6468/X6471/X6475/X6476/X6479/X6480/ |
| | | X6481/X6482/X6483/X699/X70/X700/X702/X705/X709/X710/X73/X7406/ |
| | | X7407/X7518/X7593/X7594/X7597/X7598/X7600/X7602/X7603/X7605/X7606/ |
| | | X7609/X7610/X7616/X7617/X7623/X7625/X7626/X7627/X7628/X7635/ |
| | | X7636/X7637/X7640/X7643/X854/X8560/X857/X858/X859/X8680/X8681/X8683/ |
| | | X8684/X8687/X8688/X8692/X8695/X8696/X8698/X8699/X8708/X8710/ |
| | | X8711/X8717/X8720/X8728/X8729/X8730/X90/X907/X9643/X9646/X9647/X9649/ |
| | | X9650/X9658/X9661/X9662/X9673/X9675/X9676/X9690 |
| ASN_seq_aaDown_R | 1.28 | X1191/X1193/X1206/X1207/X1208/X1266/X1267/X1277/X1281/X1287/X1869/ |
| | | X1883/X1884/X193/X194/X195/X1952/X1953/X196/X1974/X1978/X198/X23/ |
| | | X239/X25/X2756/X2827/X2828/X2868/X3865/X3947/X397/X399/X401/X402/ |
| | | X406/X407/X462/X714/X715/X717/X719/X727/X728/X729/X75/X76/X78/X787/X793 |
| ASN_seq_aaDown_S | 1.28 | X1194/X1867/X25/X2743 |
| ASN_seq_aaDown_T | 1.28 | X1164/X1169/X1172/X1176/X1179/X1184/X1355/X1479/X17/X1837/X184/X1840/ |
| | | X1843/X1852/X1857/X187/X189/X19/X192/X2054/X21/X2212/X2708/ |
| | | X2713/X2734/X3/X3762/X385/X388/X390/X392/X394/X68/X696/X70/X700/ |
| | | X703/X705/X708/X710/X73/X853 |
| ASN_seq_aaDown_W | 1.28 | X1/X1166/X1184/X1832/X1847/X1852/X1860/X2704/X2705/X2724/X2727/X2734/ |
| | | X2738/X2739/X2740/X3/X3750/X3773/X3779/X3787/X49/X4928/X4950/ |
| | | X4951/X4955/X4956/X4959/X6193/X6194/X6195/X6196/X704/X7392/X7395/ |
| | | X7397/X7404/X7406/X7407/X8550/X8555/X8556/X9573 |
| ASN_seq_aaDown_Y | 1.28 | X105/X1206/X1207/X1266/X1267/X1283/X1284/X1883/X1952/X1953/X198/ |
| | | X1980/X241/X25/X2827/X2828/X406/X407/X461/X464/X727/X728/X729/X78/ |
| | | X789/X792/X795 |
| ASN_seq_aaUp_A | 1.28 | X1/X10426/X10428/X10462/X10463/X10465/X10469/X10470/X10471/X10472/ |
| | | X10473/X10474/X10483/X10484/X10485/X10494/X10495/X11108/X11112/ |
| | | X11117/X11118/X11119/X11585/X1162/X1163/X1164/X1165/X1167/X1168/ |
| | | X1169/X1170/X1171/X1172/X1175/X1177/X1180/X1183/X1185/X1186/X1219/ |
| | | X1221/X1222/X1223/X1224/X1225/X1226/X1228/X1229/X1230/X1231/X1232/ |
| | | X1233/X1234/X1235/X1236/X1237/X1238/X1239/X1240/X1356/X1358/ |
| | | X1359/X1361/X1365/X1431/X1432/X1433/X1435/X1437/X1439/X17/X1831/ |
| | | X1832/X1833/X1834/X1835/X1836/X1837/X1839/X184/X1840/X1841/X1842/ |
| | | X1844/X1848/X185/X1851/X1853/X1854/X1856/X1858/X1862/X1895/X1898/ |
| | | X1899/X190/X1901/X1902/X1903/X1904/X1905/X1906/X1907/X1908/X1910/ |
| | | X1911/X1912/X1913/X1914/X1915/X1916/X1917/X1918/X1919/X1920/ |
| | | X1921/X1922/X1923/X1924/X1925/X2035/X2036/X2055/X2056/X2057/X2062/ |
| | | X2063/X2064/X2066/X2067/X2069/X2120/X2145/X2146/X2148/X2149/X215/ |
| | | X2150/X2151/X2152/X2154/X2157/X216/X2161/X2163/X217/X218/X26/ |
| | | X2699/X2700/X2701/X2702/X2704/X2705/X2706/X2707/X2709/X2712/X2713/ |
| | | X2715/X2716/X2717/X2718/X2723/X2725/X2729/X2733/X2735/X2736/X2766/ |
| | | X2768/X2769/X2770/X2771/X2773/X2774/X2775/X2776/X2777/X2779/ |
| | | X2780/X2781/X2782/X2783/X2785/X2786/X2788/X2789/X2790/X2791/X2792/ |
| | | X2793/X2794/X2795/X2796/X2797/X2798/X2799/X2922/X2923/X2924/X2926/ |
| | | X2927/X2928/X2944/X2946/X2950/X2951/X2952/X2953/X2955/X2956/ |
| | | X2957/X2958/X2959/X2960/X2962/X2996/X2997/X3002/X3004/X3032/X3033/ |
| | | X3034/X3035/X3036/X3038/X3039/X3040/X3042/X3044/X3045/X3046/X3050/ |
| | | X3051/X3052/X3053/X3059/X3060/X3062/X3064/X3066/X3072/X3074/ |
| | | X32/X3742/X3743/X3744/X3746/X3747/X3749/X3750/X3752/X3753/X3754/ |
| | | X3755/X3760/X3761/X3763/X3766/X3767/X3768/X3769/X3773/X3774/X3776/ |
| | | X3777/X3784/X3805/X3811/X3813/X3814/X3815/X3816/X3817/X3819/X3820/ |
| | | X3822/X3823/X3825/X3826/X3827/X3828/X3829/X383/X3830/X3831/X3832/ |
| | | X3833/X3834/X3835/X3836/X385/X387/X389/X391/X395/X3966/X3968/ |
| | | X3970/X3972/X3973/X3974/X3976/X3977/X3978/X3992/X3993/X3994/X3995/ |
| | | X3996/X3997/X3998/X4000/X4001/X4002/X4005/X4006/X4007/X4008/ |
| | | X4009/X4025/X4026/X4032/X4033/X4037/X4038/X4039/X4040/X4042/X4066/ |
| | | X4067/X4069/X4070/X4071/X4072/X4073/X4074/X4076/X4078/X4079/X4080/ |
| | | X4081/X4082/X4083/X4087/X4088/X4089/X4090/X4093/X4095/X4097/ |
| | | X4098/X4099/X4102/X4103/X4105/X4108/X4109/X4111/X4112/X4116/X4117/ |
| | | X4123/X4125/X4129/X4130/X4131/X4132/X4133/X4134/X424/X425/X426/ |
| | | X427/X428/X429/X430/X4915/X4916/X4917/X4919/X4920/X4921/X4922/X4923/ |
| | | X4924/X4926/X4927/X4928/X4929/X4932/X4933/X4934/X4935/X4939/ |
| | | X4940/X4941/X4942/X4947/X4948/X4950/X4951/X4952/X4957/X4974/X4976/ |
| | | X4978/X4979/X4980/X4981/X4982/X4984/X4985/X4987/X4988/X4989/X4990/ |
| | | X4991/X511/X5120/X5123/X5125/X5127/X5128/X5129/X5131/X5133/ |
| | | X5134/X5135/X5141/X5142/X5143/X5144/X5145/X5147/X5148/X5150/X5151/ |
| | | X5152/X5153/X5154/X5155/X5161/X5164/X5165/X5169/X5170/X5171/X5172/ |
| | | X5173/X5174/X5178/X5179/X5180/X5181/X5200/X5201/X5203/X5204/ |
| | | X5205/X5206/X5208/X5210/X5211/X5212/X5213/X5214/X5217/X5220/X5221/ |
| | | X5222/X5225/X5227/X5229/X5230/X5231/X5232/X5233/X5237/X5238/X5239/ |
| | | X5242/X5243/X5244/X5245/X5248/X5249/X5252/X5253/X5254/X5255/ |
| | | X5259/X5262/X5265/X5267/X5268/X5269/X5270/X5275/X5276/X5277/X5278/ |
| | | X5279/X5280/X5282/X5284/X5285/X5286/X5287/X5288/X5289/X5290/X5293/ |
| | | X532/X6159/X6160/X6161/X6162/X6163/X6164/X6165/X6167/X6168/X6170/ |
| | | X6171/X6173/X6174/X6175/X6176/X6181/X6182/X6184/X6185/X6186/ |
| | | X6187/X6191/X6194/X6201/X6205/X6207/X6208/X6211/X6325/X6328/X6331/ |
| | | X6334/X6335/X6336/X6338/X6339/X6344/X6346/X6347/X6348/X6349/X6350/ |
| | | X6351/X6352/X6353/X6355/X6356/X6357/X6358/X6359/X6363/X6364/ |
| | | X6365/X6366/X6367/X6368/X6369/X6381/X6383/X6384/X6387/X6391/X6394/ |
| | | X6395/X6397/X6399/X6400/X6401/X6406/X6407/X6409/X6413/X6414/X6415/ |
| | | X6418/X6419/X6420/X6422/X6424/X6426/X6427/X6428/X6430/X6431/ |
| | | X6432/X6433/X6438/X6445/X6447/X6448/X6449/X6450/X6453/X6454/X6455/ |
| | | X6456/X6457/X6458/X6459/X6461/X6462/X6465/X6466/X6467/X6468/X6469/ |
| | | X6470/X6471/X6475/X6476/X6477/X6478/X6479/X6480/X6481/X6482/ |
| | | X6483/X6484/X6490/X6491/X68/X694/X696/X697/X699/X7/X701/X702/X703/ |
| | | X706/X709/X71/X711/X7379/X7380/X7381/X7382/X7384/X7385/X7387/ |
| | | X7388/X7389/X7390/X7392/X7394/X7395/X7396/X7397/X7401/X7403/X7404/ |
| | | X744/X746/X747/X748/X749/X750/X751/X7510/X7513/X7514/X7515/X7517/ |
| | | X7518/X7519/X752/X7520/X7521/X7522/X7524/X7525/X7526/X7527/X7528/ |
| | | X7529/X753/X7530/X754/X7541/X755/X7550/X7551/X7556/X7557/X756/ |
| | | X7560/X7563/X7564/X7568/X7570/X7571/X7572/X7575/X7576/X7577/X7578/ |
| | | X7591/X7594/X7595/X7596/X7597/X7598/X7599/X7600/X7602/X7603/ |
| | | X7604/X7605/X7606/X7607/X7608/X7609/X7610/X7611/X7615/X7616/X7617/ |
| | | X7618/X7619/X7620/X7621/X7623/X7625/X7626/X7627/X7628/X7629/X7635/ |
| | | X7636/X7637/X7638/X7639/X7640/X7643/X7651/X7652/X854/X8542/X8543/ |
| | | X8545/X8546/X8547/X8548/X855/X8550/X8552/X8553/X8555/X8556/ |
| | | X8558/X858/X8631/X8633/X8634/X8635/X8636/X8654/X8655/X8660/X8661/ |
| | | X8664/X8676/X8678/X8680/X8681/X8682/X8683/X8684/X8685/X8686/X8687/ |
| | | X8688/X8689/X8692/X8693/X8694/X8695/X8696/X8697/X8698/X8699/X8700/ |
| | | X8701/X8707/X8708/X8709/X8710/X8711/X8712/X8713/X8714/X8715/ |
| | | X8716/X8717/X8720/X8728/X8729/X8730/X8731/X8742/X901/X903/X92/X93/ |
| | | X94/X9567/X9569/X9570/X9571/X9573/X9575/X9630/X9639/X9640/X9641/ |
| | | X9643/X9644/X9645/X9646/X9647/X9648/X9649/X9650/X9651/X9652/X9658/ |
| | | X9659/X9660/X9661/X9662/X9663/X9672/X9673/X9674/X9675/X9676/ |
| | | X9677/X9678/X9689/X9690 |
| ASN_seq_aaUp_C | 1.28 | X1/X1163/X1167/X1171/X1219/X1223/X1224/X1225/X1226/X1231/X1240/X16/ |
| | | X183/X1832/X1834/X1836/X1842/X1848/X1899/X1904/X1907/X1908/X1914/ |
| | | X1919/X216/X217/X26/X2700/X2705/X2707/X2712/X2718/X2725/X2736/ |
| | | X2771/X2777/X2782/X2799/X3743/X3749/X3750/X3755/X3761/X3768/X3769/ |
| | | X3777/X3829/X383/X386/X424/X426/X427/X4923/X4926/X4927/X4929/ |
| | | X4934/X4935/X4942/X4947/X6164/X6170/X6173/X6174/X6176/X6181/X6186/ |
| | | X6187/X67/X697/X7/X704/X7384/X7389/X7394/X7396/X7397/X7403/X744/ |
| | | X748/X749/X751/X752/X8547/X8552/X8555/X92/X9570 |
| ASN_seq_aaUp_D | 1.28 | X1180/X1858/X190/X389/X701/X71 |
| ASN_seq_aaUp_E | 1.28 | X105/X1199/X1202/X1204/X1283/X1284/X1357/X1358/X1360/X1361/X1362/ |
| | | X1363/X1364/X1365/X187/X1876/X1878/X192/X197/X1980/X2056/X2058/ |
| | | X2059/X2060/X2061/X2062/X2064/X2065/X2066/X2067/X2068/X2069/X21/ |
| | | X214/X24/X241/X265/X2751/X29/X2945/X2947/X2948/X2949/X2950/X2952/ |
| | | X2954/X2955/X2956/X2958/X2959/X2960/X2961/X2962/X3991/X3994/X3997/ |
| | | X3999/X4000/X4001/X4003/X4004/X4005/X4007/X4008/X4009/X403/X461/ |
| | | X464/X510/X511/X512/X5143/X5146/X5148/X5149/X5150/X5152/X5153/ |
| | | X5155/X6/X6195/X6345/X6347/X6350/X6351/X708/X722/X725/X73/X7518/ |
| | | X77/X789/X792/X795/X84/X854/X856/X8560/X857/X858/X859/X90/X9575 |
| ASN_seq_aaUp_F | 1.28 | X83 |
| ASN_seq_aaUp_G | 1.28 | X1189/X1194/X1867/X2743/X400/X718 |
| ASN_seq_aaUp_H | 1.28 | X1/X1206/X1207/X1208/X1224/X1226/X1240/X1266/X1277/X1281/X1287/X1883/ |
| | | X1884/X1908/X1919/X1952/X1961/X1974/X1978/X198/X216/X239/X25/ |
| | | X26/X2756/X2799/X2827/X2855/X2868/X3907/X406/X407/X424/X427/X462/ |
| | | X49/X6/X7/X727/X728/X729/X744/X749/X752/X78/X787/X793/X92 |
| ASN_seq_aaUp_I | 1.28 | X117/X1206/X1207/X1208/X1266/X1267/X13/X1354/X1883/X1884/X1952/X1953/ |
| | | X198/X25/X263/X2756/X2827/X2828/X3865/X3947/X406/X407/X45/X508/ |
| | | X509/X6/X727/X728/X729/X78/X851/X852 |
| ASN_seq_aaUp_K | 1.28 | X117/X1188/X1190/X1192/X1193/X13/X1354/X1355/X1865/X1868/X1894/X193/ |
| | | X194/X195/X196/X2054/X23/X263/X2741/X2763/X3802/X397/X398/X399/ |
| | | X401/X402/X45/X508/X509/X713/X714/X715/X716/X719/X75/X76/X851/ |
| | | X852/X853 |
| ASN_seq_aaUp_L | 1.28 | X1179/X1184/X1193/X1277/X1281/X1287/X1289/X1356/X1357/X1359/X1360/ |
| | | X1362/X1363/X1364/X1440/X1852/X1860/X189/X193/X194/X195/X196/X1974/ |
| | | X1976/X1978/X1984/X2055/X2057/X2058/X2059/X2060/X2061/X2063/ |
| | | X2064/X2065/X2068/X214/X2162/X2166/X23/X239/X254/X265/X2727/X2734/ |
| | | X2738/X2861/X2868/X2870/X29/X2944/X2945/X2946/X2947/X2948/X2949/ |
| | | X2951/X2953/X2954/X2957/X2959/X2961/X3065/X3070/X3073/X3775/X3779/ |
| | | X388/X3922/X394/X397/X399/X3991/X3992/X3993/X3995/X3996/X3998/ |
| | | X3999/X4002/X4003/X4004/X4006/X4007/X401/X402/X4114/X4119/X4124/ |
| | | X4127/X4130/X462/X489/X4956/X4959/X510/X512/X5141/X5142/X5144/ |
| | | X5145/X5146/X5147/X5148/X5149/X5151/X5154/X5264/X5273/X5276/X5283/ |
| | | X6196/X6344/X6345/X6346/X6348/X6349/X6352/X6452/X6463/X6476/X70/ |
| | | X705/X714/X715/X719/X75/X7517/X7519/X7520/X7593/X76/X7617/X787/ |
| | | X793/X84/X855/X856/X8560/X857/X859/X8716/X90/X907 |
| ASN_seq_aaUp_N | 1.28 | X1168/X1170/X1175/X1182/X1183/X1184/X1185/X1186/X1835/X1839/X185/ |
| | | X1850/X1851/X1852/X1853/X1856/X1860/X1861/X1862/X2064/X2700/X2704/ |
| | | X2723/X2727/X2728/X2729/X2732/X2733/X2734/X2735/X2738/X2739/X2740/ |
| | | X2959/X3743/X3766/X3773/X3774/X3775/X3776/X3779/X3780/X3781/ |
| | | X3782/X3783/X3784/X3787/X387/X391/X4007/X4926/X4928/X4932/X4950/ |
| | | X4951/X4952/X4955/X4956/X4957/X4958/X4959/X5148/X6173/X6174/X6175/ |
| | | X6184/X6191/X6193/X6194/X6195/X6196/X6197/X699/X702/X709/X7394/ |
| | | X7395/X7397/X7401/X7406/X7407/X8555/X8556/X8558/X8560/X9575 |
| ASN_seq_aaUp_P | 1.28 | X1162/X1163/X1164/X1165/X1169/X1170/X1171/X1172/X1175/X1176/X1177/ |
| | | X1180/X1183/X1185/X1219/X1222/X1223/X1224/X1225/X1226/X1228/X1229/ |
| | | X1230/X1231/X1232/X1233/X1234/X1235/X1236/X1237/X1238/X1239/ |
| | | X1240/X1322/X1431/X1432/X1433/X1435/X1437/X1831/X1833/X1834/X1836/ |
| | | X1837/X184/X1840/X1842/X1844/X185/X1851/X1853/X1857/X1858/X1898/ |
| | | X1899/X190/X1901/X1902/X1903/X1904/X1905/X1906/X1907/X1908/X1910/ |
| | | X1911/X1912/X1913/X1914/X1915/X1916/X1917/X1918/X1919/X1920/X1921/ |
| | | X1922/X1923/X1924/X1925/X1961/X2016/X2022/X2035/X2145/X2146/ |
| | | X2148/X2149/X215/X2150/X2151/X2152/X2154/X2157/X216/X217/X218/X26/ |
| | | X2699/X2700/X2701/X2702/X2703/X2707/X2709/X2712/X2713/X2716/X2717/ |
| | | X2718/X2733/X2735/X2768/X2769/X2770/X2771/X2773/X2774/X2775/ |
| | | X2776/X2777/X2779/X2780/X2781/X2782/X2783/X2785/X2786/X2788/X2789/ |
| | | X2790/X2791/X2792/X2793/X2794/X2795/X2796/X2797/X2798/X2799/X2855/ |
| | | X2900/X2914/X2922/X2923/X2924/X2927/X3032/X3033/X3034/X3035/ |
| | | X3036/X3038/X3039/X3040/X3042/X3044/X3045/X3046/X3050/X3051/X3052/ |
| | | X3053/X3059/X3060/X3062/X32/X3742/X3744/X3745/X3746/X3747/X3749/ |
| | | X3753/X3754/X3755/X3761/X3763/X3805/X3811/X3813/X3814/X3815/X3816/ |
| | | X3817/X3819/X3820/X3822/X3823/X3825/X3826/X3827/X3828/X3829/ |
| | | X383/X3830/X3831/X3832/X3833/X3834/X3835/X3836/X385/X389/X3907/ |
| | | X391/X395/X3954/X3959/X3966/X3968/X3970/X3972/X3973/X3974/X3976/ |
| | | X4066/X4067/X4069/X4070/X4071/X4072/X4073/X4074/X4076/X4078/X4079/ |
| | | X4080/X4082/X4083/X4087/X4088/X4089/X4090/X4095/X4097/X4098/X4099/ |
| | | X4102/X4103/X4105/X4109/X4111/X4112/X424/X425/X426/X427/X428/ |
| | | X429/X430/X4915/X4916/X4917/X4919/X4920/X4921/X4922/X4923/X4924/ |
| | | X4927/X4929/X4940/X4942/X4974/X4976/X4978/X4979/X4980/X4982/X4984/ |
| | | X4985/X4987/X4988/X4989/X4990/X4991/X5107/X5120/X5123/X5125/X5127/ |
| | | X5128/X5129/X5131/X5134/X5200/X5201/X5203/X5206/X5208/X5210/ |
| | | X5211/X5212/X5213/X5214/X5220/X5221/X5222/X5227/X5229/X5230/X5231/ |
| | | X5232/X5233/X5237/X5238/X5239/X5242/X5245/X5249/X5252/X5253/X5255/ |
| | | X5259/X532/X6159/X6160/X6161/X6162/X6163/X6164/X6165/X6167/ |
| | | X6168/X6171/X6205/X6207/X6208/X6211/X6325/X6328/X6334/X6335/X6336/ |
| | | X6338/X6383/X6384/X6394/X6395/X6397/X6399/X6400/X6401/X6406/X6409/ |
| | | X6413/X6418/X6424/X6426/X6427/X6428/X6433/X6438/X694/X696/X697/ |
| | | X699/X7/X701/X703/X706/X71/X710/X711/X7379/X7380/X7381/X7382/ |
| | | X7385/X7387/X7388/X7389/X7390/X744/X746/X747/X748/X749/X750/X751/ |
| | | X7510/X7513/X7515/X752/X753/X754/X7541/X755/X7550/X7556/X756/X7563/ |
| | | X7564/X7575/X8542/X8543/X8545/X8546/X8553/X8631/X8660/X901/X903/ |
| | | X92/X93/X94/X9567/X9569 |
| ASN_seq_aaUp_Q | 1.28 | X1184/X1356/X1358/X1359/X1361/X1365/X1852/X187/X192/X2055/X2056/ |
| | | X2057/X2062/X2063/X2066/X2067/X2069/X21/X2734/X2944/X2946/X2950/ |
| | | X2951/X2952/X2953/X2955/X2956/X2957/X2958/X2960/X2962/X390/X392/ |
| | | X3992/X3993/X3994/X3995/X3996/X3997/X3998/X4000/X4001/X4002/X4005/ |
| | | X4006/X4008/X4009/X511/X5141/X5142/X5143/X5144/X5145/X5147/X5150/ |
| | | X5151/X5152/X5153/X5154/X5155/X6344/X6346/X6347/X6348/X6349/X6350/ |
| | | X6351/X6352/X708/X73/X7517/X7518/X7519/X7520/X854/X855/X858/X8716 |
| ASN_seq_aaUp_R | 1.28 | X1274/X1971/X2857/X83 |
| ASN_seq_aaUp_S | 1.28 | X1166/X1167/X1168/X1170/X1174/X1175/X1177/X1180/X1183/X1185/X1206/ |
| | | X1207/X1266/X1267/X16/X183/X1835/X1847/X1848/X1849/X185/X1851/ |
| | | X1853/X1856/X1858/X1860/X1861/X1883/X190/X1952/X1953/X198/X2704/ |
| | | X2723/X2724/X2725/X2727/X2728/X2733/X2735/X2827/X2828/X3773/X3774/ |
| | | X3775/X3776/X3783/X386/X3865/X387/X389/X391/X3947/X395/X396/X406/ |
| | | X407/X49/X4950/X4951/X6193/X67/X698/X699/X701/X702/X704/X706/ |
| | | X707/X709/X71/X711/X727/X728/X729/X78 |
| ASN_seq_aaUp_T | 1.28 | X1188/X1189/X1190/X1191/X1192/X1194/X1865/X1867/X1868/X1894/X2741/ |
| | | X2743/X2763/X3802/X398/X400/X713/X716/X717/X718 |
| ASN_seq_aaUp_V | 1.28 | X117/X1179/X1188/X1190/X1192/X13/X1354/X1355/X1464/X1865/X1868/X187/ |
| | | X1881/X189/X19/X192/X2054/X21/X2186/X263/X2741/X2748/X2754/X3117/ |
| | | X3791/X3795/X388/X392/X394/X398/X45/X49/X4961/X508/X509/X70/ |
| | | X700/X705/X713/X716/X73/X851/X852/X853 |
| ASN_seq_aaUp_W | 1.28 | X1224/X1226/X1240/X1908/X1914/X1919/X1951/X216/X26/X2777/X2799/X2826/ |
| | | X3829/X3864/X424/X427/X7/X744/X749/X752/X92 |
| ASN_seq_aaUp_Y | 1.28 | X104/X110/X1329/X234/X235/X255/X39/X452/X453/X491/X492/X799/X830/X831 |
| ASN_seq_SS_sspro8C | 1.28 | X1/X10425/X10426/X10454/X10457/X10458/X10459/X10460/X10461/X105/ |
| | | X10656/X10670/X10679/X10695/X10700/X10702/X11095/X11249/X11422/X1162/ |
| | | X1163/X1164/X1165/X1166/X1167/X1168/X1169/X1170/X1171/X1172/ |
| | | X1174/X1175/X1176/X1177/X1179/X1180/X1181/X1182/X1183/X1184/X1185/ |
| | | X1186/X1219/X1220/X1227/X1283/X1284/X1289/X1356/X1358/X1359/X1361/ |
| | | X1362/X1363/X1364/X1365/X1557/X16/X17/X183/X1831/X1832/X1833/ |
| | | X1834/X1835/X1836/X1837/X1839/X184/X1840/X1841/X1842/X1843/X1844/ |
| | | X1847/X1848/X1849/X185/X1850/X1851/X1852/X1853/X1854/X1856/X1857/ |
| | | X1858/X1860/X1861/X1862/X187/X189/X1896/X1897/X1899/X19/X190/ |
| | | X1900/X1909/X192/X1976/X1980/X2055/X2056/X2057/X2060/X2062/X2063/ |
| | | X2064/X2065/X2066/X2067/X2068/X2069/X21/X2160/X2331/X2332/X241/ |
| | | X265/X2699/X2700/X2701/X2702/X2703/X2704/X2705/X2706/X2707/X2708/ |
| | | X2709/X2712/X2713/X2715/X2716/X2717/X2718/X2723/X2724/X2725/X2727/ |
| | | X2728/X2729/X2732/X2733/X2734/X2735/X2736/X2738/X2739/X2764/ |
| | | X2765/X2767/X2771/X2772/X2778/X2784/X2787/X2870/X29/X2925/X2944/ |
| | | X2946/X2947/X2948/X2950/X2951/X2952/X2953/X2955/X2956/X2957/X2958/ |
| | | X2959/X2960/X2961/X2962/X3/X3056/X3061/X3072/X3285/X3286/X3287/ |
| | | X3293/X3294/X3299/X3332/X3742/X3743/X3744/X3745/X3746/X3747/X3749/ |
| | | X3750/X3752/X3753/X3754/X3755/X3760/X3761/X3762/X3763/X3766/X3767/ |
| | | X3768/X3769/X3773/X3774/X3775/X3776/X3777/X3779/X3780/X3781/ |
| | | X3782/X3783/X3784/X3803/X3804/X3806/X3807/X3808/X3809/X3810/X3812/ |
| | | X3818/X3821/X3824/X383/X3833/X385/X386/X387/X388/X389/X390/X391/ |
| | | X392/X394/X395/X396/X3967/X3969/X3975/X3992/X3993/X3994/X3995/ |
| | | X3996/X3997/X3998/X3999/X4000/X4001/X4002/X4003/X4005/X4006/X4007/ |
| | | X4008/X4009/X4075/X4096/X4104/X4110/X4113/X4123/X4130/X4132/X4158/ |
| | | X4373/X4374/X4376/X4378/X4380/X4384/X4385/X4389/X4391/X4392/ |
| | | X4394/X4397/X4442/X461/X464/X4915/X4916/X4917/X4919/X4920/X4921/ |
| | | X4922/X4923/X4924/X4926/X4927/X4928/X4929/X4932/X4933/X4934/X4935/ |
| | | X4939/X4940/X4941/X4942/X4947/X4948/X4950/X4951/X4952/X4956/X4957/ |
| | | X4958/X4968/X4969/X4970/X4971/X4972/X4973/X4975/X4977/X4982/ |
| | | X4983/X4986/X511/X5119/X512/X5124/X5130/X5132/X5141/X5142/X5143/ |
| | | X5144/X5145/X5147/X5148/X5149/X5150/X5151/X5152/X5153/X5154/X5155/ |
| | | X5202/X5207/X5215/X5218/X5219/X5228/X5240/X5241/X5250/X5256/X5257/ |
| | | X5258/X5260/X5276/X5278/X5282/X5289/X5309/X5310/X5315/X5564/ |
| | | X5565/X5566/X5567/X5568/X5570/X5572/X5575/X5577/X5579/X5580/X5583/ |
| | | X5585/X5586/X5588/X5590/X5591/X5594/X5596/X5598/X5604/X5659/X6159/ |
| | | X6160/X6161/X6162/X6163/X6164/X6165/X6167/X6168/X6170/X6171/ |
| | | X6173/X6174/X6175/X6176/X6181/X6182/X6184/X6185/X6186/X6187/X6191/ |
| | | X6194/X6199/X6200/X6202/X6203/X6204/X6206/X6209/X6210/X6329/X6330/ |
| | | X6332/X6337/X6344/X6345/X6346/X6347/X6348/X6349/X6350/X6351/ |
| | | X6352/X6382/X6385/X6386/X6388/X6389/X6392/X6393/X6396/X6402/X6403/ |
| | | X6404/X6405/X6408/X6416/X6417/X6425/X6434/X6435/X6436/X6437/X6439/ |
| | | X6440/X6441/X6442/X6443/X6461/X6470/X6476/X6478/X6503/X6506/ |
| | | X6507/X6510/X67/X6760/X6761/X6762/X6763/X6764/X6765/X6767/X6769/ |
| | | X6771/X6772/X6773/X6775/X6777/X6780/X6782/X6784/X6787/X6789/X6791/ |
| | | X6793/X6795/X6796/X6798/X68/X6805/X6807/X6812/X694/X696/X697/ |
| | | X698/X699/X70/X700/X701/X702/X703/X704/X705/X706/X707/X708/X709/ |
| | | X71/X710/X711/X73/X7379/X7380/X7381/X7382/X7384/X7385/X7387/X7388/ |
| | | X7389/X7390/X7392/X7394/X7395/X7396/X7397/X7401/X7403/X7404/X7408/ |
| | | X7409/X7411/X7412/X745/X7507/X7511/X7516/X7517/X7518/X7519/X7520/ |
| | | X7537/X7538/X7539/X7540/X7542/X7543/X7544/X7545/X7546/X7547/ |
| | | X7549/X7552/X7553/X7554/X7555/X7558/X7561/X7562/X7566/X7569/X7573/ |
| | | X7574/X7579/X7580/X7581/X7582/X7583/X7584/X7585/X7586/X7587/X7588/ |
| | | X7589/X7617/X7620/X7639/X7662/X7664/X7665/X789/X7911/X7912/ |
| | | X7913/X7914/X7915/X7916/X7918/X792/X7920/X7922/X7925/X7926/X7928/ |
| | | X7930/X7931/X7936/X7942/X7945/X7946/X7948/X795/X7955/X7957/X7961/ |
| | | X7963/X7965/X7967/X7972/X7974/X7978/X7992/X83/X854/X8542/X8543/ |
| | | X8545/X8546/X8547/X8548/X855/X8550/X8552/X8553/X8555/X8556/X8558/ |
| | | X857/X858/X859/X8632/X8640/X8641/X8642/X8643/X8644/X8645/X8646/ |
| | | X8647/X8648/X8649/X8651/X8652/X8653/X8656/X8657/X8658/X8659/X8662/ |
| | | X8663/X8665/X8666/X8667/X8668/X8669/X8670/X8671/X8672/X8673/X8674/ |
| | | X8675/X8714/X8716/X8975/X8976/X8977/X8979/X8980/X8985/X8987/ |
| | | X8989/X8990/X8995/X90/X9000/X9003/X9008/X9014/X9016/X9018/X9020/ |
| | | X9025/X9027/X9031/X9036/X9038/X9041/X9043/X9046/X9566/X9567/X9569/ |
| | | X9570/X9571/X9573/X9619/X9620/X9621/X9622/X9623/X9624/X9625/X9627/ |
| | | X9628/X9629/X9631/X9632/X9633/X9634/X9635/X9636/X9637/X9897/ |
| | | X9898/X9903/X9909/X9912/X9917/X9926/X9932/X9937/X9939/X9943/X9948/ |
| | | X9950/X9967/X9969/X9972 |
| ASN_seq_SS_sspro8E | 1.28 | X1189/X1191/X1193/X1194/X1355/X1464/X1867/X1869/X193/X194/X195/X196/ |
| | | X2054/X2186/X23/X2743/X3117/X397/X399/X400/X401/X402/X49/X714/ |
| | | X715/X717/X718/X719/X75/X76/X853 |
| ASN_seq_SS_sspro8H | 1.28 | X1184/X1852/X2734/X8560/X9575 |
| ASN_seq_SS_sspro8S | 1.28 | X1368/X1415/X1417/X2124/X2125/X3007/X466/X49/X6/X798/X895 |
| ASN_seq_SS_sspro8T | 1.28 | X104/X110/X1177/X1186/X1224/X1226/X1240/X1328/X1329/X1330/X1379/ |
| | | X1383/X190/X1908/X1919/X2078/X2082/X2083/X216/X234/X235/X255/X26/ |
| | | X2799/X2966/X389/X39/X395/X424/X427/X452/X453/X491/X492/X493/X7/ |
| | | X706/X71/X711/X744/X749/X752/X799/X829/X830/X831/X832/X871/X92 |
| ASN_seq_SS_ssproE | 1.28 | X104/X1188/X1189/X1190/X1191/X1192/X1193/X1194/X1464/X1865/X1867/ |
| | | X1868/X1869/X1880/X1894/X193/X194/X195/X196/X2186/X23/X234/X235/ |
| | | X2741/X2743/X2753/X2763/X3116/X3117/X3794/X3802/X39/X397/X398/X399/ |
| | | X400/X401/X402/X452/X49/X713/X714/X715/X716/X717/X718/X719/X75/X76 |
| ASN_seq_SS_ssproH | 1.28 | X1184/X1852/X2734/X8560/X9575 |
| ASN_struct_aa_A | 1.28 | X10425/X10426/X10427/X10428/X10462/X10463/X10465/X10469/X10470/X10471/ |
| | | X10472/X10473/X10474/X10483/X10484/X10485/X10494/X10495/X11095/ |
| | | X11096/X11108/X11112/X11117/X11118/X11119/X11581/X11585/X1162/ |
| | | X1163/X1164/X1165/X1166/X1167/X1168/X1169/X1170/X1171/X1172/X1174/ |
| | | X1175/X1176/X1177/X1180/X1181/X1182/X1183/X1185/X1186/X1356/ |
| | | X1358/X1359/X1361/X1362/X1363/X1364/X1365/X1439/X17/X1831/X1832/ |
| | | X1833/X1834/X1835/X1836/X1837/X1839/X184/X1840/X1841/X1842/X1843/ |
| | | X1844/X1847/X1848/X1849/X185/X1850/X1851/X1853/X1854/X1856/X1857/ |
| | | X1858/X1860/X1861/X1862/X189/X190/X2055/X2056/X2057/X2060/X2062/ |
| | | X2063/X2064/X2065/X2066/X2067/X2068/X2069/X2120/X2161/X2163/X265/ |
| | | X2699/X2700/X2701/X2702/X2703/X2704/X2705/X2706/X2707/X2708/ |
| | | X2709/X2712/X2713/X2715/X2716/X2717/X2723/X2724/X2725/X2727/X2728/ |
| | | X2729/X2732/X2733/X2735/X2736/X2738/X2739/X2740/X29/X2944/X2946/ |
| | | X2948/X2950/X2951/X2952/X2953/X2955/X2956/X2957/X2958/X2959/X2960/ |
| | | X2961/X2962/X2996/X2997/X3/X3002/X3004/X3064/X3066/X3072/X3074/ |
| | | X3742/X3743/X3744/X3745/X3746/X3747/X3749/X3750/X3752/X3753/X3754/ |
| | | X3760/X3761/X3762/X3763/X3766/X3767/X3768/X3769/X3773/X3774/ |
| | | X3776/X3777/X3779/X3780/X3781/X3782/X3783/X3784/X3787/X383/X385/ |
| | | X387/X388/X389/X390/X391/X392/X395/X396/X3992/X3993/X3994/X3995/ |
| | | X3996/X3997/X3998/X4000/X4001/X4002/X4003/X4005/X4006/X4007/X4008/ |
| | | X4009/X4025/X4026/X4032/X4033/X4037/X4038/X4039/X4040/X4042/X4116/ |
| | | X4117/X4123/X4125/X4129/X4130/X4131/X4133/X4134/X4915/X4916/ |
| | | X4917/X4919/X4920/X4921/X4922/X4923/X4924/X4926/X4927/X4928/X4929/ |
| | | X4932/X4933/X4934/X4935/X4939/X4940/X4941/X4947/X4948/X4950/X4951/ |
| | | X4952/X4955/X4956/X4957/X4958/X4959/X511/X512/X5141/X5142/X5143/ |
| | | X5144/X5145/X5147/X5148/X5150/X5151/X5152/X5153/X5154/X5155/ |
| | | X5161/X5164/X5165/X5169/X5170/X5171/X5172/X5173/X5174/X5178/X5179/ |
| | | X5180/X5181/X5262/X5265/X5267/X5268/X5269/X5270/X5275/X5276/X5277/ |
| | | X5279/X5280/X5282/X5284/X5285/X5286/X5287/X5288/X5290/X5293/ |
| | | X6159/X6160/X6161/X6162/X6163/X6164/X6165/X6167/X6168/X6170/X6171/ |
| | | X6173/X6174/X6175/X6176/X6181/X6182/X6184/X6185/X6186/X6187/X6191/ |
| | | X6193/X6194/X6195/X6196/X6197/X6344/X6346/X6347/X6348/X6349/ |
| | | X6350/X6351/X6352/X6353/X6355/X6356/X6357/X6358/X6359/X6363/X6364/ |
| | | X6365/X6366/X6367/X6368/X6369/X6445/X6447/X6448/X6449/X6450/X6453/ |
| | | X6454/X6455/X6456/X6457/X6458/X6459/X6461/X6462/X6465/X6466/ |
| | | X6467/X6468/X6469/X6471/X6475/X6476/X6477/X6479/X6480/X6481/X6482/ |
| | | X6483/X6484/X6490/X6491/X68/X694/X696/X697/X699/X70/X700/X701/ |
| | | X702/X703/X706/X707/X708/X709/X71/X710/X711/X7379/X7380/X7381/X7382/ |
| | | X7384/X7385/X7387/X7388/X7389/X7390/X7392/X7393/X7394/X7395/ |
| | | X7396/X7397/X7401/X7403/X7404/X7406/X7407/X7517/X7518/X7519/X7520/ |
| | | X7521/X7522/X7524/X7525/X7526/X7527/X7528/X7529/X7530/X7591/X7594/ |
| | | X7595/X7596/X7597/X7598/X7599/X7600/X7602/X7603/X7604/X7605/ |
| | | X7606/X7607/X7608/X7609/X7610/X7611/X7615/X7616/X7617/X7618/X7619/ |
| | | X7621/X7623/X7625/X7626/X7627/X7628/X7629/X7635/X7636/X7637/X7638/ |
| | | X7640/X7643/X7651/X7652/X854/X8542/X8543/X8544/X8545/X8546/ |
| | | X8547/X8548/X855/X8550/X8551/X8552/X8553/X8555/X8556/X8558/X8560/ |
| | | X857/X858/X859/X8633/X8634/X8635/X8636/X8676/X8678/X8680/X8681/ |
| | | X8682/X8683/X8684/X8685/X8686/X8687/X8688/X8689/X8692/X8693/X8694/ |
| | | X8695/X8696/X8697/X8698/X8699/X8700/X8701/X8707/X8708/X8709/X8710/ |
| | | X8711/X8712/X8713/X8715/X8716/X8717/X8720/X8728/X8729/X8730/ |
| | | X8731/X8742/X90/X9566/X9567/X9568/X9569/X9570/X9571/X9573/X9574/ |
| | | X9575/X9639/X9640/X9641/X9643/X9644/X9645/X9646/X9647/X9648/X9649/ |
| | | X9650/X9651/X9652/X9658/X9659/X9660/X9661/X9662/X9663/X9672/X9673/ |
| | | X9674/X9675/X9676/X9677/X9678/X9689/X9690 |
| ASN_struct_aa_C | 1.28 | X1/X10425/X10426/X10427/X11095/X11581/X1162/X1163/X1164/X1165/X1166/ |
| | | X1167/X1169/X1170/X1171/X1172/X1174/X1175/X1177/X1180/X1181/ |
| | | X1183/X1185/X1186/X17/X1831/X1832/X1833/X1834/X1835/X1836/X1837/ |
| | | X184/X1840/X1841/X1842/X1843/X1844/X1847/X1848/X1849/X185/X1851/ |
| | | X1853/X1854/X1856/X1857/X1858/X1862/X190/X2699/X2700/X2701/X2702/ |
| | | X2703/X2704/X2705/X2706/X2707/X2708/X2709/X2712/X2713/X2715/X2716/ |
| | | X2717/X2718/X2723/X2724/X2725/X2729/X2733/X2735/X2736/X3742/X3743/ |
| | | X3744/X3745/X3746/X3747/X3749/X3750/X3752/X3753/X3754/X3755/ |
| | | X3760/X3761/X3762/X3763/X3766/X3768/X3769/X3774/X3776/X3777/X3784/ |
| | | X383/X385/X387/X389/X391/X395/X396/X4915/X4916/X4917/X4919/X4920/ |
| | | X4921/X4922/X4923/X4924/X4926/X4927/X4928/X4929/X4932/X4934/ |
| | | X4935/X4939/X4940/X4941/X4942/X4947/X4948/X4951/X4952/X6/X6159/X6160/ |
| | | X6161/X6162/X6163/X6164/X6165/X6167/X6168/X6170/X6171/X6173/ |
| | | X6174/X6175/X6176/X6181/X6182/X6184/X6186/X6187/X68/X694/X696/X697/ |
| | | X698/X699/X701/X703/X706/X707/X709/X71/X711/X7379/X7380/X7381/ |
| | | X7382/X7384/X7385/X7387/X7388/X7389/X7390/X7394/X7396/X7403/X7404/ |
| | | X8542/X8543/X8544/X8545/X8546/X8547/X8548/X8552/X8553/X9566/ |
| | | X9567/X9568/X9569/X9570/X9571 |
| ASN_struct_aa_D | 1.28 | X1/X1162/X1163/X1164/X1169/X1171/X1172/X1177/X1180/X1219/X1222/X1223/ |
| | | X1224/X1225/X1226/X1228/X1229/X1230/X1231/X1232/X1233/X1234/ |
| | | X1235/X1236/X1237/X1238/X1239/X1240/X1431/X1432/X1433/X1435/X1437/ |
| | | X17/X1831/X1834/X1836/X1837/X184/X1840/X1842/X1844/X1858/X1898/ |
| | | X1899/X190/X1901/X1902/X1903/X1904/X1905/X1906/X1907/X1908/X1910/ |
| | | X1911/X1912/X1913/X1914/X1915/X1916/X1917/X1918/X1919/X1920/X1921/ |
| | | X1922/X1923/X1924/X1925/X2035/X2145/X2146/X2148/X2149/X215/ |
| | | X2150/X2151/X2152/X2154/X2157/X216/X217/X218/X26/X2699/X2700/X2702/ |
| | | X2707/X2709/X2712/X2713/X2716/X2718/X2735/X2768/X2769/X2770/X2771/ |
| | | X2773/X2774/X2775/X2776/X2777/X2779/X2780/X2781/X2782/X2783/ |
| | | X2785/X2786/X2788/X2789/X2790/X2791/X2792/X2793/X2794/X2795/X2796/ |
| | | X2797/X2798/X2799/X2922/X2923/X2924/X2927/X3032/X3033/X3034/X3035/ |
| | | X3036/X3038/X3039/X3040/X3042/X3044/X3045/X3046/X3050/X3051/ |
| | | X3052/X3053/X3059/X3060/X3062/X32/X3742/X3744/X3746/X3749/X3753/ |
| | | X3755/X3761/X3763/X3805/X3811/X3813/X3814/X3815/X3816/X3817/X3819/ |
| | | X3820/X3822/X3823/X3825/X3826/X3827/X3828/X3829/X383/X3830/X3831/ |
| | | X3832/X3833/X3834/X3835/X3836/X385/X389/X395/X3966/X3968/X3970/ |
| | | X3972/X3973/X3974/X3976/X4066/X4067/X4069/X4070/X4071/X4072/X4073/ |
| | | X4074/X4076/X4078/X4079/X4080/X4082/X4083/X4087/X4088/X4089/ |
| | | X4090/X4095/X4097/X4098/X4099/X4102/X4103/X4105/X4109/X4111/X4112/ |
| | | X424/X425/X426/X427/X428/X429/X430/X4916/X4919/X4923/X4924/X4927/ |
| | | X4940/X4942/X4974/X4976/X4978/X4979/X4980/X4982/X4984/X4985/ |
| | | X4987/X4988/X4989/X4990/X4991/X5120/X5123/X5125/X5127/X5128/X5129/ |
| | | X5131/X5134/X5200/X5201/X5203/X5206/X5208/X5210/X5211/X5212/X5213/ |
| | | X5214/X5220/X5221/X5222/X5227/X5229/X5230/X5231/X5232/X5233/ |
| | | X5237/X5238/X5239/X5242/X5245/X5249/X5252/X5253/X5255/X5259/X532/ |
| | | X6164/X6165/X6167/X6205/X6207/X6208/X6211/X6325/X6328/X6334/X6335/ |
| | | X6336/X6338/X6383/X6384/X6394/X6395/X6397/X6399/X6400/X6401/X6406/ |
| | | X6409/X6413/X6418/X6424/X6426/X6427/X6428/X6433/X6438/X68/X696/ |
| | | X697/X7/X701/X703/X706/X71/X711/X7380/X7389/X7390/X744/X746/ |
| | | X747/X748/X749/X750/X751/X7510/X7513/X7515/X752/X753/X754/X7541/ |
| | | X755/X7550/X7556/X756/X7563/X7564/X7575/X8542/X8631/X8660/X901/X903/ |
| | | X92/X93/X94/X9569 |
| ASN_struct_aa_E | 1.28 | X10494/X1206/X1207/X1208/X1266/X1267/X1317/X1320/X1321/X1322/X1440/ |
| | | X1883/X1884/X1952/X1953/X198/X2012/X2016/X2021/X2022/X2023/X2120/ |
| | | X2162/X2166/X25/X253/X254/X2756/X2827/X2828/X2900/X2910/X2913/ |
| | | X2914/X2996/X2997/X3002/X3004/X3065/X3070/X3072/X3073/X3865/X3947/ |
| | | X3954/X3958/X3959/X4025/X4026/X4032/X4033/X4037/X4038/X4039/ |
| | | X4040/X4042/X406/X407/X4114/X4119/X4123/X4124/X4127/X4130/X4132/ |
| | | X487/X489/X5107/X5161/X5164/X5165/X5169/X5170/X5171/X5172/X5173/ |
| | | X5174/X5178/X5179/X5180/X5181/X5264/X5273/X5276/X5278/X5282/X5283/ |
| | | X5289/X5293/X6/X6353/X6355/X6356/X6357/X6358/X6359/X6363/X6364/ |
| | | X6365/X6366/X6367/X6368/X6369/X6452/X6461/X6462/X6463/X6470/X6476/ |
| | | X6478/X6490/X6491/X727/X728/X729/X7521/X7522/X7524/X7525/X7526/ |
| | | X7527/X7528/X7529/X7530/X7593/X7615/X7617/X7618/X7620/X7621/X7639/ |
| | | X7651/X7652/X78/X823/X824/X8560/X8633/X8634/X8635/X8636/X8707/ |
| | | X8709/X8712/X8714/X8715/X8742/X907/X9575/X9672/X9674/X9677/X9689 |
| ASN_struct_aa_F | 1.28 | X1206/X1207/X1208/X1266/X1267/X1883/X1884/X1952/X1953/X198/X25/X2756/ |
| | | X2827/X2828/X3865/X3947/X406/X407/X727/X728/X729/X78 |
| ASN_struct_aa_G | 1.28 | X104/X110/X1206/X1207/X1208/X1266/X1267/X1316/X1317/X1318/X1320/ |
| | | X1321/X1322/X1329/X1379/X1883/X1884/X1952/X1953/X198/X2011/X2012/ |
| | | X2013/X2015/X2016/X2017/X2021/X2022/X2023/X2078/X234/X235/X25/X253/ |
| | | X255/X2756/X2827/X2828/X2895/X2899/X2900/X2901/X2904/X2910/X2913/ |
| | | X2914/X3865/X39/X3946/X3947/X3953/X3954/X3955/X3958/X3959/X406/ |
| | | X407/X452/X453/X485/X487/X491/X492/X5106/X5107/X5108/X6/X727/ |
| | | X728/X729/X78/X799/X822/X823/X824/X830/X831/X871 |
| ASN_struct_aa_H | 1.28 | X1/X10425/X10426/X10427/X10459/X11095/X1162/X1163/X1164/X1165/X1167/ |
| | | X1169/X117/X1170/X1171/X1172/X1175/X1176/X1177/X1179/X1180/X1183/ |
| | | X1185/X1186/X1206/X1207/X1208/X1219/X1220/X1221/X1222/X1223/ |
| | | X1224/X1225/X1226/X1227/X1228/X1229/X1230/X1231/X1232/X1233/X1234/ |
| | | X1235/X1236/X1237/X1238/X1239/X1240/X1266/X1267/X13/X1354/X1355/ |
| | | X1431/X1432/X1433/X1435/X1437/X17/X1831/X1832/X1833/X1834/X1836/ |
| | | X1837/X184/X1840/X1841/X1842/X1844/X185/X1851/X1853/X1854/X1856/ |
| | | X1857/X1858/X1862/X187/X1883/X1884/X189/X1895/X1896/X1897/X1898/ |
| | | X1899/X19/X190/X1900/X1901/X1902/X1903/X1904/X1905/X1906/X1907/ |
| | | X1908/X1909/X1910/X1911/X1912/X1913/X1914/X1915/X1916/X1917/X1918/ |
| | | X1919/X192/X1920/X1921/X1922/X1923/X1924/X1925/X1952/X1953/X198/ |
| | | X2035/X2036/X2054/X21/X2145/X2146/X2148/X2149/X215/X2150/X2151/ |
| | | X2152/X2154/X2157/X216/X2160/X217/X218/X25/X26/X263/X2699/X2700/ |
| | | X2701/X2702/X2703/X2705/X2706/X2707/X2709/X2712/X2713/X2715/X2716/ |
| | | X2717/X2718/X2729/X2733/X2735/X2736/X2756/X2764/X2765/X2766/ |
| | | X2767/X2768/X2769/X2770/X2771/X2772/X2773/X2774/X2775/X2776/X2777/ |
| | | X2778/X2779/X2780/X2781/X2782/X2783/X2784/X2785/X2786/X2788/X2789/ |
| | | X2790/X2791/X2792/X2793/X2794/X2795/X2796/X2797/X2798/X2799/ |
| | | X2827/X2828/X2922/X2923/X2924/X2925/X2926/X2927/X2928/X3/X3032/ |
| | | X3033/X3034/X3035/X3036/X3038/X3039/X3040/X3042/X3044/X3045/X3046/ |
| | | X3050/X3051/X3052/X3053/X3056/X3059/X3060/X3061/X3062/X32/X3742/ |
| | | X3744/X3745/X3746/X3747/X3749/X3750/X3752/X3753/X3754/X3755/X3760/ |
| | | X3761/X3763/X3768/X3784/X3803/X3804/X3805/X3806/X3807/X3808/ |
| | | X3811/X3813/X3814/X3815/X3816/X3817/X3818/X3819/X3820/X3822/X3823/ |
| | | X3824/X3825/X3826/X3827/X3828/X3829/X383/X3830/X3831/X3832/X3833/ |
| | | X3834/X3835/X3836/X385/X3865/X388/X389/X390/X391/X392/X394/X3947/ |
| | | X395/X396/X3966/X3967/X3968/X3969/X3970/X3972/X3973/X3974/X3975/ |
| | | X3976/X3977/X3978/X406/X4066/X4067/X4069/X407/X4070/X4071/X4072/ |
| | | X4073/X4074/X4075/X4076/X4078/X4079/X4080/X4081/X4082/X4083/ |
| | | X4087/X4088/X4089/X4090/X4093/X4095/X4097/X4098/X4099/X4102/X4103/ |
| | | X4104/X4105/X4108/X4109/X4111/X4112/X4113/X424/X425/X426/X427/ |
| | | X428/X429/X430/X45/X49/X4915/X4916/X4917/X4919/X4920/X4921/X4922/ |
| | | X4923/X4924/X4927/X4929/X4934/X4939/X4940/X4941/X4942/X4971/X4974/ |
| | | X4975/X4976/X4978/X4979/X4980/X4981/X4982/X4983/X4984/X4985/X4987/ |
| | | X4988/X4989/X4990/X4991/X508/X509/X5119/X5120/X5123/X5124/X5125/ |
| | | X5127/X5128/X5129/X5130/X5131/X5132/X5133/X5134/X5135/X5200/ |
| | | X5201/X5202/X5203/X5204/X5205/X5206/X5207/X5208/X5210/X5211/X5212/ |
| | | X5213/X5214/X5215/X5217/X5220/X5221/X5222/X5225/X5227/X5229/X5230/ |
| | | X5231/X5232/X5233/X5237/X5238/X5239/X5240/X5242/X5243/X5244/ |
| | | X5245/X5248/X5249/X5252/X5253/X5254/X5255/X5256/X5257/X5258/X5259/ |
| | | X5260/X532/X6/X6159/X6160/X6162/X6163/X6164/X6165/X6167/X6168/ |
| | | X6170/X6171/X6186/X6201/X6202/X6205/X6207/X6208/X6210/X6211/X6325/ |
| | | X6328/X6329/X6330/X6331/X6332/X6334/X6335/X6336/X6337/X6338/X6339/ |
| | | X6381/X6382/X6383/X6384/X6385/X6387/X6389/X6391/X6394/X6395/ |
| | | X6396/X6397/X6399/X6400/X6401/X6403/X6406/X6407/X6408/X6409/X6413/ |
| | | X6414/X6415/X6416/X6418/X6419/X6420/X6422/X6424/X6426/X6427/X6428/ |
| | | X6430/X6431/X6432/X6433/X6435/X6436/X6437/X6438/X6439/X6440/ |
| | | X6442/X6443/X6503/X68/X694/X696/X697/X699/X7/X70/X700/X701/X703/ |
| | | X705/X706/X707/X709/X71/X710/X711/X727/X728/X729/X73/X7379/X7380/ |
| | | X7381/X7382/X7384/X7385/X7387/X7388/X7389/X7390/X7412/X744/X745/ |
| | | X746/X747/X748/X749/X750/X7507/X751/X7510/X7511/X7513/X7514/X7515/ |
| | | X7516/X752/X753/X7537/X7538/X754/X7540/X7541/X7544/X7547/X755/ |
| | | X7550/X7551/X7553/X7556/X7557/X7558/X756/X7560/X7563/X7564/X7568/ |
| | | X7570/X7571/X7572/X7573/X7575/X7576/X7577/X7578/X7579/X7580/X7581/ |
| | | X7582/X7584/X7585/X7586/X7587/X7589/X7662/X78/X851/X852/X853/ |
| | | X8542/X8543/X8544/X8552/X8553/X8631/X8632/X8645/X8647/X8654/X8655/ |
| | | X8657/X8660/X8661/X8664/X8665/X8666/X8667/X8668/X8669/X8670/ |
| | | X8673/X8674/X901/X903/X9031/X92/X93/X94/X9566/X9567/X9568/X9569/ |
| | | X9624/X9630/X9631/X9632/X9634/X9635 |
| ASN_struct_aa_I | 1.28 | X1/X10428/X1162/X1163/X1164/X1165/X1169/X1171/X1172/X1181/X1219/ |
| | | X1223/X1224/X1225/X1226/X1231/X1240/X1357/X1360/X1362/X1363/X1364/ |
| | | X1464/X17/X1831/X1832/X1833/X1834/X1836/X1837/X184/X1840/X1841/ |
| | | X1842/X1843/X1844/X1899/X1904/X1907/X1908/X1914/X1919/X2035/X2058/ |
| | | X2059/X2060/X2061/X2064/X2065/X2068/X214/X216/X217/X2186/X2211/ |
| | | X26/X265/X2699/X2700/X2701/X2702/X2705/X2706/X2707/X2708/X2709/ |
| | | X2712/X2713/X2715/X2716/X2717/X2718/X2771/X2777/X2782/X2799/X29/ |
| | | X2922/X2923/X2945/X2947/X2948/X2949/X2954/X2959/X2961/X3115/X3116/ |
| | | X3117/X3742/X3743/X3744/X3746/X3747/X3749/X3750/X3752/X3753/X3754/ |
| | | X3755/X3760/X3761/X3762/X3763/X3766/X3767/X3768/X3769/X3776/ |
| | | X3829/X383/X385/X393/X3966/X3970/X3991/X3999/X4003/X4004/X4007/ |
| | | X4166/X424/X426/X427/X4915/X4916/X4917/X4919/X4921/X4923/X4926/X4927/ |
| | | X4928/X4929/X4932/X4933/X4934/X4935/X4939/X4940/X4941/X4942/ |
| | | X4947/X4948/X510/X512/X5120/X5146/X5148/X5149/X6160/X6162/X6164/ |
| | | X6167/X6168/X6170/X6173/X6174/X6175/X6176/X6181/X6182/X6184/X6185/ |
| | | X6186/X6187/X6191/X6194/X6345/X68/X694/X696/X697/X7/X703/X707/ |
| | | X708/X7381/X7382/X7384/X7387/X7389/X7392/X7394/X7395/X7396/X7397/ |
| | | X7401/X7403/X7404/X744/X748/X749/X751/X752/X84/X8545/X8547/X8550/ |
| | | X8552/X8555/X8556/X8558/X856/X857/X859/X90/X92/X9570/X9573/X9575 |
| ASN_struct_aa_K | 1.28 | X19/X3 |
| ASN_struct_aa_L | 1.28 | X1164/X1169/X1172/X1179/X1415/X17/X1837/X184/X1840/X1843/X189/X2125/ |
| | | X2708/X2713/X3762/X385/X388/X394/X68/X696/X70/X700/X703/X705/X895 |
| ASN_struct_aa_M | 1.28 | X117/X1188/X1189/X1190/X1192/X1194/X1199/X1202/X13/X1354/X1368/X1370/ |
| | | X1415/X1479/X1865/X1867/X1868/X1876/X1894/X197/X2072/X2125/ |
| | | X2212/X24/X263/X2741/X2743/X2763/X3802/X398/X400/X403/X45/X466/X508/ |
| | | X509/X5098/X713/X716/X718/X722/X725/X77/X798/X851/X852/X862/X895 |
| ASN_struct_aa_P | 1.28 | X1206/X1207/X1208/X1266/X1267/X1277/X1281/X1287/X1316/X1317/X1318/ |
| | | X1320/X1321/X1322/X1883/X1884/X1952/X1953/X1974/X1978/X198/X2011/ |
| | | X2012/X2013/X2015/X2016/X2021/X2022/X2023/X239/X25/X253/X2756/ |
| | | X2827/X2828/X2868/X2895/X2899/X2900/X2904/X2910/X2913/X2914/X3865/ |
| | | X3946/X3947/X3953/X3954/X3958/X3959/X406/X407/X462/X485/X487/ |
| | | X5106/X5107/X6/X727/X728/X729/X78/X787/X793/X822/X823/X824 |
| ASN_struct_aa_R | 1.28 | X104/X117/X1199/X1202/X1204/X13/X1354/X1355/X1876/X1878/X197/X2054/ |
| | | X234/X235/X24/X25/X263/X2748/X2751/X3791/X39/X403/X45/X452/X4961/ |
| | | X508/X509/X722/X725/X77/X799/X851/X852/X853 |
| ASN_struct_aa_S | 1.28 | X105/X1206/X1207/X1266/X1267/X1283/X1284/X16/X183/X1883/X1952/X1953/ |
| | | X198/X1980/X241/X25/X2827/X2828/X386/X3865/X406/X407/X461/X464/ |
| | | X49/X67/X698/X727/X728/X729/X78/X789/X792/X795 |
| ASN_struct_aa_T | 1.28 | X1223/X1224/X1225/X1226/X1231/X1240/X1904/X1907/X1908/X1914/X1919/ |
| | | X2035/X216/X217/X26/X2777/X2782/X2799/X2922/X2923/X3829/X3966/ |
| | | X3970/X424/X426/X427/X5120/X7/X744/X748/X749/X751/X752/X92 |
| ASN_struct_aa_V | 1.28 | X117/X1188/X1190/X1191/X1192/X1199/X1202/X1204/X13/X1354/X1355/X1865/ |
| | | X1868/X1869/X1876/X1878/X1894/X197/X2054/X24/X263/X2741/X2748/ |
| | | X2751/X2763/X3791/X3802/X398/X403/X45/X49/X4961/X508/X509/X713/ |
| | | X716/X717/X722/X725/X77/X851/X852/X853 |
| ASN_struct_aa_W | 1.28 | X1/X1162/X1163/X1164/X1169/X1171/X1172/X1176/X1179/X1181/X1219/X1222/ |
| | | X1223/X1224/X1225/X1226/X1228/X1229/X1230/X1231/X1232/X1233/ |
| | | X1234/X1235/X1236/X1237/X1238/X1239/X1240/X1431/X1435/X17/X1831/ |
| | | X1832/X1834/X1836/X1837/X184/X1840/X1842/X1843/X1844/X1857/X187/ |
| | | X189/X1898/X1899/X19/X1901/X1902/X1903/X1904/X1905/X1906/X1907/ |
| | | X1908/X1910/X1911/X1912/X1914/X1915/X1916/X1917/X1918/X1919/X1920/ |
| | | X1921/X1922/X1923/X1924/X1925/X1984/X2035/X21/X2146/X2148/X215/ |
| | | X2151/X2154/X2157/X216/X217/X218/X26/X2699/X2702/X2703/X2705/X2707/ |
| | | X2708/X2709/X2712/X2713/X2769/X2770/X2771/X2773/X2774/X2775/ |
| | | X2777/X2779/X2780/X2781/X2782/X2783/X2785/X2786/X2789/X2790/X2791/ |
| | | X2792/X2793/X2794/X2795/X2796/X2797/X2798/X2799/X2861/X2922/X2923/ |
| | | X2927/X3/X3036/X3038/X3039/X3042/X3046/X3050/X3053/X32/X3742/ |
| | | X3744/X3745/X3746/X3747/X3750/X3761/X3762/X3763/X3813/X3814/X3815/ |
| | | X3816/X3817/X3819/X3820/X3823/X3825/X3826/X3827/X3829/X383/X3830/ |
| | | X3831/X3832/X3833/X3834/X3835/X3836/X385/X388/X390/X3922/X394/ |
| | | X3966/X3970/X3972/X3974/X3976/X4067/X4070/X4074/X4079/X4083/X4087/ |
| | | X4090/X4097/X4105/X424/X425/X426/X427/X428/X429/X430/X4916/X4917/ |
| | | X4919/X4920/X4978/X4979/X4980/X4982/X4984/X4985/X4988/X4989/ |
| | | X4990/X4991/X5120/X5123/X5125/X5127/X5129/X5134/X5200/X5211/X5213/ |
| | | X5220/X5229/X5233/X5237/X5242/X5253/X532/X6167/X6168/X6207/X6208/ |
| | | X6211/X6325/X6328/X6334/X6336/X6384/X6394/X6400/X6406/X6418/ |
| | | X6426/X6433/X68/X696/X697/X7/X70/X700/X703/X705/X707/X708/X710/X73/ |
| | | X744/X746/X747/X748/X749/X750/X751/X7510/X752/X753/X754/X755/ |
| | | X7556/X756/X7563/X7575/X83/X8660/X901/X92/X93/X94 |
| ASN_struct_aa_Y | 1.28 | X49 |
| ASN_struct_SS_dsspE | 1.28 | X1188/X1189/X1190/X1191/X1192/X1193/X1194/X1355/X1464/X1479/X1865/ |
| | | X1866/X1867/X1868/X1869/X1894/X193/X194/X195/X196/X2054/X2186/ |
| | | X2212/X23/X2741/X2742/X2743/X2763/X3116/X3117/X3802/X3935/X397/X398/ |
| | | X399/X400/X401/X402/X5098/X713/X714/X715/X716/X717/X718/X719/ |
| | | X75/X76/X853 |
| ASN_struct_SS_dsspH | 1.28 | X1162/X1163/X1164/X1165/X1167/X1168/X1169/X1170/X1171/X1172/X1175/ |
| | | X1176/X1177/X1179/X1180/X1181/X1182/X1183/X1184/X1185/X1186/X17/ |
| | | X1831/X1832/X1833/X1834/X1835/X1836/X1837/X1839/X184/X1840/X1841/ |
| | | X1842/X1843/X1844/X1848/X185/X1850/X1851/X1852/X1853/X1854/X1856/ |
| | | X1857/X1858/X1860/X1861/X1862/X189/X19/X190/X2699/X2700/X2701/ |
| | | X2702/X2704/X2705/X2706/X2707/X2708/X2709/X2712/X2713/X2715/X2716/ |
| | | X2717/X2718/X2723/X2724/X2725/X2727/X2728/X2729/X2732/X2733/ |
| | | X2734/X2735/X2736/X2738/X2739/X2740/X3742/X3743/X3744/X3746/X3749/ |
| | | X3750/X3752/X3753/X3754/X3755/X3760/X3761/X3762/X3763/X3766/X3767/ |
| | | X3768/X3769/X3773/X3774/X3775/X3776/X3777/X3779/X3780/X3781/ |
| | | X3782/X3783/X3784/X3787/X383/X385/X387/X388/X389/X391/X394/X395/ |
| | | X4915/X4916/X4919/X4923/X4926/X4927/X4928/X4929/X4932/X4933/X4934/ |
| | | X4935/X4939/X4940/X4941/X4942/X4947/X4948/X4950/X4951/X4952/X4955/ |
| | | X4956/X4957/X4958/X4959/X6160/X6164/X6167/X6170/X6173/X6174/ |
| | | X6175/X6176/X6181/X6182/X6184/X6185/X6186/X6187/X6193/X6194/X6195/ |
| | | X6196/X6197/X68/X694/X696/X697/X699/X70/X700/X701/X702/X703/X705/ |
| | | X706/X709/X71/X710/X711/X7381/X7384/X7389/X7392/X7394/X7395/X7396/ |
| | | X7397/X7403/X7404/X7406/X7407/X8545/X8547/X8550/X8552/X8560/X9570/X9573 |
| ASN_struct_SS_dsspS | 1.28 | X1199/X1202/X1204/X1206/X1207/X1208/X1266/X1876/X1878/X1883/X1884/ |
| | | X1951/X1952/X197/X198/X24/X25/X2751/X2756/X2826/X2827/X3864/X3947/ |
| | | X403/X406/X407/X49/X722/X725/X727/X728/X729/X77/X78 |
| ASN_struct_SS_dsspT | 1.28 | X1206/X1267/X1328/X1951/X1953/X198/X2083/X25/X2826/X2828/X3864/X406/ |
| | | X407/X453/X727/X728/X78/X799/X829 |
| SER.THR_seq_aaAll_E | 1.28 | X237/X495/X68 |
| SER.THR_seq_aaAll_G | 1.28 | X1/X17 |
| SER.THR_seq_aaAll_H | 1.28 | X119/X149/X211/X27/X32/X380/X456/X6/X781/X90/X93 |
| SER.THR_seq_aaAll_L | 1.28 | X1/X1080/X17/X237/X495/X68 |
| SER.THR_seq_aaAll_M | 1.28 | X1 |
| SER.THR_seq_aaAll_P | 1.28 | X2/X28/X87 |
| SER.THR_seq_aaAll_Q | 1.28 | X1/X17 |
| SER.THR_seq_aaAll_R | 1.28 | X27 |
| SER.THR_seq_aaAll_S | 1.28 | X1 |
| SER.THR_seq_aaAll_Y | 1.28 | X1/X119/X149/X211/X27/X32/X380/X456/X6/X781/X90/X93 |
| SER.THR_seq_aaDown_G | 1.28 | X1 |
| SER.THR_seq_aaDown_L | 1.28 | X237/X495/X68 |
| SER.THR_seq_aaDown_Q | 1.28 | X1 |
| SER.THR_seq_aaDown_S | 1.28 | X1/X237/X495/X68 |
| SER.THR_seq_aaDown_Y | 1.28 | X119/X149/X211/X27/X32/X380/X456/X6/X781/X90/X93 |
| SER.THR_seq_aaUp_H | 1.28 | X119/X149/X211/X27/X32/X380/X456/X6/X781/X90/X93 |
| SER.THR_seq_aaUp_L | 1.28 | X1/X1080/X17/X237/X495/X68 |
| SER.THR_struct_aa_F | 1.28 | X29 |
| SER.THR_struct_aa_I | 1.28 | X208/X86 |
| SER.THR_struct_aa_L | 1.28 | X1/X17/X237/X495/X68 |
| SER.THR_struct_aa_Q | 1.28 | X1/X17 |
| ASN_seq_aaAll_A | 2.33 | X10459/X1219/X1220/X1221/X1222/X1223/X1224/X1225/X1226/X1227/X1228/ |
| | | X1229/X1230/X1231/X1232/X1233/X1234/X1235/X1236/X1237/X1238/X1239/ |
| | | X1240/X1431/X1432/X1433/X1435/X1437/X1557/X170/X1895/X1896/ |
| | | X1897/X1898/X1899/X1900/X1901/X1902/X1903/X1904/X1905/X1906/X1907/ |
| | | X1908/X1909/X1910/X1911/X1912/X1913/X1914/X1915/X1916/X1917/X1918/ |
| | | X1919/X1920/X1921/X1922/X1923/X1924/X1925/X2035/X2036/X2145/ |
| | | X2146/X2148/X2149/X215/X2150/X2151/X2152/X2154/X2157/X216/X2160/ |
| | | X217/X218/X2331/X2332/X26/X2764/X2765/X2766/X2767/X2768/X2769/X2770/ |
| | | X2771/X2772/X2773/X2774/X2775/X2776/X2777/X2778/X2779/X2780/ |
| | | X2781/X2782/X2783/X2784/X2785/X2786/X2787/X2788/X2789/X2790/X2791/ |
| | | X2792/X2793/X2794/X2795/X2796/X2797/X2798/X2799/X2922/X2923/X2924/ |
| | | X2925/X2926/X2927/X2928/X3032/X3033/X3034/X3035/X3036/X3038/ |
| | | X3039/X3040/X3042/X3044/X3045/X3046/X3050/X3051/X3052/X3053/X3056/ |
| | | X3059/X3060/X3061/X3062/X32/X3285/X3286/X3287/X3293/X3803/X3804/ |
| | | X3805/X3806/X3807/X3808/X3810/X3811/X3812/X3813/X3814/X3815/X3816/ |
| | | X3817/X3818/X3819/X3820/X3821/X3822/X3823/X3824/X3825/X3826/ |
| | | X3827/X3828/X3829/X3830/X3831/X3832/X3833/X3834/X3835/X3836/X3966/ |
| | | X3967/X3968/X3969/X3970/X3972/X3973/X3974/X3975/X3976/X3977/X3978/ |
| | | X4066/X4067/X4069/X4070/X4071/X4072/X4073/X4074/X4075/X4076/ |
| | | X4078/X4079/X4080/X4081/X4082/X4083/X4087/X4088/X4089/X4090/X4093/ |
| | | X4095/X4096/X4097/X4098/X4099/X4102/X4103/X4104/X4105/X4108/X4109/ |
| | | X4111/X4112/X4113/X424/X425/X426/X427/X428/X429/X430/X4373/X4374/ |
| | | X4376/X4378/X4384/X4389/X4392/X4394/X4969/X4970/X4971/X4973/ |
| | | X4974/X4975/X4976/X4977/X4978/X4979/X4980/X4981/X4982/X4983/X4984/ |
| | | X4985/X4986/X4987/X4988/X4989/X4990/X4991/X5119/X5120/X5123/X5124/ |
| | | X5125/X5127/X5128/X5129/X5130/X5131/X5132/X5133/X5134/X5135/ |
| | | X5200/X5201/X5202/X5203/X5204/X5205/X5206/X5207/X5208/X5210/X5211/ |
| | | X5212/X5213/X5214/X5215/X5217/X5218/X5219/X5220/X5221/X5222/X5225/ |
| | | X5227/X5228/X5229/X5230/X5231/X5232/X5233/X5237/X5238/X5239/ |
| | | X5240/X5241/X5242/X5243/X5244/X5245/X5248/X5249/X5252/X5253/X5254/ |
| | | X5255/X5256/X5257/X5258/X5259/X5260/X5309/X5310/X532/X5564/X5565/ |
| | | X5566/X5570/X5575/X5580/X5583/X5586/X5588/X5590/X5594/X6200/ |
| | | X6201/X6202/X6204/X6205/X6206/X6207/X6208/X6210/X6211/X6325/X6328/ |
| | | X6329/X6330/X6331/X6332/X6334/X6335/X6336/X6337/X6338/X6339/X6381/ |
| | | X6382/X6383/X6384/X6385/X6386/X6387/X6388/X6389/X6391/X6392/ |
| | | X6393/X6394/X6395/X6396/X6397/X6399/X6400/X6401/X6403/X6404/X6405/ |
| | | X6406/X6407/X6408/X6409/X6413/X6414/X6415/X6416/X6417/X6418/X6419/ |
| | | X6420/X6422/X6424/X6425/X6426/X6427/X6428/X6430/X6431/X6432/ |
| | | X6433/X6434/X6435/X6436/X6437/X6438/X6439/X6440/X6441/X6442/X6443/ |
| | | X6503/X6506/X6507/X6760/X6762/X6764/X6767/X6772/X6773/X6777/X6787/ |
| | | X6793/X6796/X6798/X6812/X7/X7409/X7412/X744/X745/X746/X747/X748/ |
| | | X749/X750/X7507/X751/X7510/X7511/X7513/X7514/X7515/X7516/X752/ |
| | | X753/X7537/X7538/X7539/X754/X7540/X7541/X7544/X7545/X7547/X7549/ |
| | | X755/X7550/X7551/X7553/X7554/X7555/X7556/X7557/X7558/X756/X7560/ |
| | | X7561/X7562/X7563/X7564/X7566/X7568/X7570/X7571/X7572/X7573/X7574/ |
| | | X7575/X7576/X7577/X7578/X7579/X7580/X7581/X7582/X7583/X7584/ |
| | | X7585/X7586/X7587/X7589/X7662/X7664/X7665/X7912/X7914/X7916/X7922/ |
| | | X7926/X7931/X7946/X7948/X7961/X8631/X8632/X8642/X8645/X8646/X8647/ |
| | | X8648/X8651/X8654/X8655/X8657/X8658/X8659/X8660/X8661/X8664/ |
| | | X8665/X8666/X8667/X8668/X8669/X8670/X8673/X8674/X8977/X8979/X8990/ |
| | | X9000/X901/X903/X9031/X9041/X9043/X92/X93/X94/X9624/X9625/X9630/ |
| | | X9631/X9632/X9634/X9635/X9903/X9932 |
| ASN_seq_aaAll_C | 2.33 | X115/X12/X1294/X1295/X1296/X1350/X1351/X1352/X1995/X1996/X2050/X2051/ |
| | | X2287/X245/X260/X27/X2887/X2941/X3226/X4295/X44/X470/X504/X5096/ |
| | | X80/X801/X802/X83/X846/X847 |
| ASN_seq_aaAll_E | 2.33 | X1208/X1344/X1345/X1346/X1347/X1884/X2016/X2042/X2043/X2044/X2045/ |
| | | X258/X2756/X2900/X2932/X2933/X2934/X3947/X3954/X3980/X3981/X502/ |
| | | X5107/X74/X843/X844 |
| ASN_seq_aaAll_F | 2.33 | X1970/X2843/X2851/X3885/X3892/X5054 |
| ASN_seq_aaAll_H | 2.33 | X1294/X1295/X1296/X170/X1995/X1996/X245/X27/X2887/X470/X80/X801/X802 |
| ASN_seq_aaAll_I | 2.33 | X10462/X10463/X10465/X10466/X10469/X10470/X10471/X10472/X10473/X10474/ |
| | | X10475/X10477/X10478/X10479/X10480/X10481/X10483/X10484/X10485/ |
| | | X10486/X10487/X10488/X10489/X10491/X10492/X10493/X10495/X10497/ |
| | | X10498/X10500/X10502/X10504/X10506/X10507/X10509/X10512/X10514/ |
| | | X10517/X10519/X10524/X11108/X11109/X11112/X11114/X11117/X11118/ |
| | | X11119/X11120/X11121/X11122/X11123/X11125/X11126/X11127/X11128/ |
| | | X11129/X11130/X11131/X11132/X11135/X11136/X11138/X11140/X11142/ |
| | | X11144/X11147/X113/X11585/X11587/X11590/X11593/X11594/X11595/X11596/ |
| | | X11597/X11599/X11600/X11601/X11603/X11605/X11898/X11901/X11904/ |
| | | X11905/X11906/X12091/X1439/X1440/X157/X170/X2161/X2162/X2163/ |
| | | X2166/X254/X3063/X3064/X3065/X3066/X3070/X3073/X3074/X393/X4114/ |
| | | X4115/X4116/X4117/X4118/X4119/X4124/X4125/X4127/X4129/X4130/X4131/ |
| | | X4133/X4134/X489/X5262/X5263/X5264/X5265/X5266/X5267/X5268/X5269/ |
| | | X5270/X5273/X5275/X5276/X5277/X5279/X5280/X5283/X5284/X5285/X5286/ |
| | | X5287/X5288/X5290/X5292/X6445/X6446/X6447/X6448/X6449/X6450/ |
| | | X6451/X6452/X6453/X6454/X6455/X6456/X6457/X6458/X6459/X6463/X6465/ |
| | | X6466/X6467/X6468/X6469/X6471/X6473/X6475/X6476/X6477/X6479/X6480/ |
| | | X6481/X6482/X6483/X6484/X6485/X6488/X6489/X6519/X7591/X7592/ |
| | | X7593/X7594/X7595/X7596/X7597/X7598/X7599/X7600/X7601/X7602/X7603/ |
| | | X7604/X7605/X7606/X7607/X7608/X7609/X7610/X7611/X7612/X7616/X7617/ |
| | | X7619/X7623/X7625/X7626/X7627/X7628/X7629/X7630/X7633/X7634/ |
| | | X7635/X7636/X7637/X7638/X7640/X7642/X7643/X7645/X7646/X7648/X7650/ |
| | | X7666/X7669/X7677/X8676/X8678/X8679/X8680/X8681/X8682/X8683/X8684/ |
| | | X8685/X8686/X8687/X8688/X8689/X8690/X8692/X8693/X8694/X8695/ |
| | | X8696/X8697/X8698/X8699/X8700/X8701/X8702/X8703/X8704/X8705/X8708/ |
| | | X8710/X8711/X8713/X8717/X8719/X8720/X8722/X8723/X8725/X8727/X8728/ |
| | | X8729/X8730/X8731/X8732/X8735/X8736/X8738/X8739/X8741/X8749/ |
| | | X8751/X8755/X8760/X8766/X907/X9639/X9640/X9641/X9643/X9644/X9645/ |
| | | X9646/X9647/X9648/X9649/X9650/X9651/X9652/X9653/X9654/X9655/X9656/ |
| | | X9658/X9659/X9660/X9661/X9662/X9663/X9664/X9666/X9667/X9668/ |
| | | X9669/X9670/X9673/X9675/X9676/X9678/X9679/X9682/X9683/X9685/X9686/ |
| | | X9688/X9690/X9692/X9693/X9695/X9697/X9699/X9703/X9705/X9707/X9710/ |
| | | X9716/X9720 |
| ASN_seq_aaAll_K | 2.33 | X1195/X1198/X1870/X1873/X1875/X188/X2745/X2747/X2761/X3153/X3790/ |
| | | X3799/X3801/X4196/X4966/X4967/X5349/X7073/X7088/X720 |
| ASN_seq_aaAll_L | 2.33 | X105/X1274/X1277/X1281/X1283/X1284/X1287/X1971/X1974/X1978/X1980/ |
| | | X239/X241/X2857/X2868/X3909/X461/X462/X464/X5073/X787/X789/X792/X793/X795 |
| ASN_seq_aaAll_M | 2.33 | X1316/X1317/X1318/X1320/X1321/X1322/X2011/X2012/X2013/X2015/X2016/ |
| | | X2017/X2018/X2021/X2022/X2023/X2024/X253/X2895/X2896/X2899/X2900/ |
| | | X2901/X2902/X2904/X2906/X2907/X2908/X2910/X2912/X2913/X2914/X3244/ |
| | | X3940/X3943/X3946/X3949/X3950/X3951/X3953/X3954/X3955/X3956/ |
| | | X3958/X3959/X3960/X4315/X485/X487/X5100/X5103/X5106/X5107/X5108/ |
| | | X5109/X5111/X5112/X5113/X5115/X5499/X6314/X6317/X6320/X6321/X6322/ |
| | | X6693/X7503/X822/X823/X824 |
| ASN_seq_aaAll_Q | 2.33 | X69 |
| ASN_seq_aaAll_R | 2.33 | X113/X115/X12/X1274/X1294/X1295/X1296/X1350/X1351/X1352/X170/X1961/ |
| | | X1971/X1991/X1995/X1996/X2049/X2050/X2051/X2287/X245/X260/X27/ |
| | | X2855/X2857/X2882/X2887/X2940/X2941/X3226/X3907/X3909/X3936/X3987/ |
| | | X4295/X44/X470/X504/X5073/X5470/X80/X801/X802/X846/X847 |
| ASN_seq_aaAll_S | 2.33 | X1266/X1267/X170/X1952/X1953/X2827/X2828/X3865/X3947/X49 |
| ASN_seq_aaAll_V | 2.33 | X1880/X2748/X2753/X3791/X3794/X3938/X4961 |
| ASN_seq_aaAll_W | 2.33 | X105/X115/X1189/X1194/X12/X1274/X1277/X1281/X1283/X1284/X1287/X1289/ |
| | | X1350/X1351/X1352/X1866/X1867/X1950/X1951/X1961/X1971/X1974/ |
| | | X1976/X1978/X1980/X1984/X2049/X2050/X2051/X2287/X239/X241/X260/X2742/ |
| | | X2743/X2825/X2826/X2855/X2857/X2861/X2868/X2870/X2940/X2941/ |
| | | X3226/X3863/X3864/X3907/X3909/X3922/X3935/X3987/X400/X4295/X44/ |
| | | X461/X462/X464/X504/X5073/X5098/X5318/X5470/X718/X787/X789/X792/ |
| | | X793/X795/X83/X846/X847 |
| ASN_seq_aaAll_Y | 2.33 | X113/X1344/X1345/X1346/X1347/X2041/X2042/X2043/X2044/X2045/X258/ |
| | | X2931/X2932/X2933/X2934/X3979/X3980/X3981/X502/X5320/X843/X844 |
| ASN_seq_aaDown_A | 2.33 | X115/X12/X1294/X1295/X1296/X1350/X1351/X1352/X1995/X1996/X2050/X2051/ |
| | | X2287/X245/X260/X27/X2887/X2941/X3226/X4295/X44/X470/X504/X80/ |
| | | X801/X802/X846/X847 |
| ASN_seq_aaDown_C | 2.33 | X1277/X1281/X1287/X1296/X188/X1961/X1974/X1978/X1991/X1995/X239/ |
| | | X2855/X2868/X2882/X2887/X3907/X3909/X3936/X462/X5073/X787/X793/X83 |
| ASN_seq_aaDown_D | 2.33 | X1344/X1345/X1346/X1347/X2042/X2043/X2044/X2045/X258/X2932/X2933/ |
| | | X2934/X3980/X3981/X502/X843/X844 |
| ASN_seq_aaDown_F | 2.33 | X1366/X1369/X1417/X1430/X2070/X2071/X2124/X2143/X2231/X3007/X3030/ |
| | | X3131/X3133/X4174/X861 |
| ASN_seq_aaDown_G | 2.33 | X1366/X1369/X1430/X2070/X2071/X2143/X2231/X3030/X3131/X3133/X4174/X861 |
| ASN_seq_aaDown_H | 2.33 | X117/X1223/X1224/X1225/X1226/X1231/X1240/X13/X1354/X1355/X1904/X1907/ |
| | | X1908/X1919/X2054/X216/X217/X26/X263/X2782/X2799/X424/X426/ |
| | | X427/X45/X508/X509/X7/X708/X744/X748/X749/X751/X752/X851/X852/X853/X92 |
| ASN_seq_aaDown_I | 2.33 | X10462/X10463/X10464/X10465/X10466/X10467/X10468/X10469/X10470/X10471/ |
| | | X10472/X10473/X10474/X10475/X10476/X10477/X10478/X10479/X10480/ |
| | | X10481/X10482/X10483/X10484/X10485/X10486/X10487/X10488/X10489/ |
| | | X10490/X10491/X10492/X10493/X10494/X10495/X10496/X10497/X10498/ |
| | | X10499/X10500/X10501/X10502/X10503/X10504/X10505/X10506/X10507/ |
| | | X10508/X10509/X10510/X10512/X10513/X10514/X10517/X10519/X10523/ |
| | | X10524/X10527/X10529/X10538/X10549/X10558/X10568/X11108/X11109/ |
| | | X11110/X11111/X11112/X11113/X11114/X11115/X11116/X11117/X11118/X11119/ |
| | | X11120/X11121/X11122/X11123/X11124/X11125/X11126/X11127/X11128/ |
| | | X11129/X11130/X11131/X11132/X11133/X11134/X11135/X11136/X11137/ |
| | | X11138/X11139/X11140/X11142/X11144/X11147/X11150/X11163/X11174/ |
| | | X11180/X11584/X11585/X11586/X11587/X11588/X11589/X11590/X11591/ |
| | | X11592/X11593/X11594/X11595/X11596/X11597/X11598/X11599/X11600/ |
| | | X11601/X11602/X11603/X11605/X11618/X11627/X11897/X11898/X11899/ |
| | | X11900/X11901/X11902/X11903/X11904/X11905/X11906/X11916/X12090/X12091/ |
| | | X12092/X12093/X12200/X1356/X1359/X1439/X1440/X1441/X170/X2055/ |
| | | X2057/X2063/X2120/X2161/X2162/X2163/X2164/X2165/X2166/X2243/ |
| | | X254/X2944/X2946/X2951/X2953/X2957/X2996/X2997/X3002/X3004/X3063/ |
| | | X3064/X3065/X3066/X3068/X3069/X3070/X3071/X3072/X3073/X3074/X3075/ |
| | | X3148/X3184/X393/X3992/X3993/X3995/X3996/X3998/X4002/X4006/X4025/ |
| | | X4026/X4032/X4033/X4037/X4038/X4039/X4040/X4042/X4114/X4115/ |
| | | X4116/X4117/X4118/X4119/X4122/X4123/X4124/X4125/X4126/X4127/X4128/ |
| | | X4129/X4130/X4131/X4132/X4133/X4134/X4136/X4137/X4138/X4188/X4190/ |
| | | X4232/X489/X5141/X5142/X5144/X5145/X5147/X5151/X5154/X5161/X5164/ |
| | | X5165/X5169/X5170/X5171/X5172/X5173/X5174/X5178/X5179/X5180/ |
| | | X5181/X5261/X5262/X5263/X5264/X5265/X5266/X5267/X5268/X5269/X5270/ |
| | | X5272/X5273/X5274/X5275/X5276/X5277/X5278/X5279/X5280/X5281/X5282/ |
| | | X5283/X5284/X5285/X5286/X5287/X5288/X5289/X5290/X5291/X5292/ |
| | | X5293/X5297/X5298/X5299/X5300/X5301/X5302/X5334/X5336/X5338/X5345/ |
| | | X5387/X5388/X6344/X6346/X6348/X6349/X6352/X6353/X6355/X6356/X6357/ |
| | | X6358/X6359/X6363/X6364/X6365/X6366/X6367/X6368/X6369/X6444/ |
| | | X6445/X6446/X6447/X6448/X6449/X6450/X6451/X6452/X6453/X6454/X6455/ |
| | | X6456/X6457/X6458/X6459/X6460/X6461/X6462/X6463/X6465/X6466/X6467/ |
| | | X6468/X6469/X6470/X6471/X6472/X6473/X6474/X6475/X6476/X6477/ |
| | | X6478/X6479/X6480/X6481/X6482/X6483/X6484/X6485/X6486/X6487/X6488/ |
| | | X6489/X6490/X6491/X6495/X6496/X6497/X6498/X6499/X6500/X6501/X6502/ |
| | | X6515/X6517/X6518/X6519/X6523/X6524/X6529/X6534/X6567/X6568/ |
| | | X6569/X7517/X7519/X7520/X7521/X7522/X7524/X7525/X7526/X7527/X7528/ |
| | | X7529/X7530/X7590/X7591/X7592/X7593/X7594/X7595/X7596/X7597/X7598/ |
| | | X7599/X7600/X7601/X7602/X7603/X7604/X7605/X7606/X7607/X7608/ |
| | | X7609/X7610/X7611/X7612/X7613/X7614/X7615/X7616/X7617/X7618/X7619/ |
| | | X7620/X7621/X7623/X7625/X7626/X7627/X7628/X7629/X7630/X7631/X7632/ |
| | | X7633/X7634/X7635/X7636/X7637/X7638/X7639/X7640/X7641/X7642/ |
| | | X7643/X7644/X7645/X7646/X7647/X7648/X7649/X7650/X7651/X7652/X7654/ |
| | | X7655/X7656/X7657/X7658/X7659/X7660/X7661/X7666/X7667/X7668/X7669/ |
| | | X7673/X7674/X7676/X7677/X7681/X7686/X7690/X7692/X7720/X7721/ |
| | | X7722/X7723/X855/X8633/X8634/X8635/X8636/X8676/X8677/X8678/X8679/ |
| | | X8680/X8681/X8682/X8683/X8684/X8685/X8686/X8687/X8688/X8689/X8690/ |
| | | X8691/X8692/X8693/X8694/X8695/X8696/X8697/X8698/X8699/X8700/ |
| | | X8701/X8702/X8703/X8704/X8705/X8706/X8707/X8708/X8709/X8710/X8711/ |
| | | X8712/X8713/X8714/X8715/X8716/X8717/X8718/X8719/X8720/X8721/X8722/ |
| | | X8723/X8724/X8725/X8726/X8727/X8728/X8729/X8730/X8731/X8732/ |
| | | X8733/X8734/X8735/X8736/X8737/X8738/X8739/X8740/X8741/X8742/X8743/ |
| | | X8744/X8745/X8746/X8747/X8748/X8749/X8750/X8751/X8754/X8755/X8759/ |
| | | X8760/X8764/X8765/X8766/X8769/X8773/X8775/X8778/X8800/X8801/ |
| | | X8802/X8811/X907/X9638/X9639/X9640/X9641/X9642/X9643/X9644/X9645/ |
| | | X9646/X9647/X9648/X9649/X9650/X9651/X9652/X9653/X9654/X9655/X9656/ |
| | | X9657/X9658/X9659/X9660/X9661/X9662/X9663/X9664/X9665/X9666/X9667/ |
| | | X9668/X9669/X9670/X9671/X9672/X9673/X9674/X9675/X9676/X9677/ |
| | | X9678/X9679/X9680/X9681/X9682/X9683/X9684/X9685/X9686/X9687/X9688/ |
| | | X9689/X9690/X9691/X9692/X9693/X9694/X9695/X9696/X9697/X9698/X9699/ |
| | | X9700/X9701/X9702/X9703/X9705/X9706/X9707/X9710/X9714/X9715/ |
| | | X9716/X9719/X9720/X9724/X9727/X9729/X9744/X9745/X9750/X9765 |
| ASN_seq_aaDown_L | 2.33 | X1344/X1345/X1346/X1347/X1416/X170/X2041/X2042/X2043/X2044/X2045/ |
| | | X2123/X258/X2931/X2932/X2933/X2934/X3006/X3979/X3980/X3981/X502/ |
| | | X5320/X843/X844 |
| ASN_seq_aaDown_M | 2.33 | X1266/X1267/X1316/X1317/X1318/X1320/X1321/X1322/X1367/X1371/X1952/ |
| | | X1953/X2011/X2012/X2013/X2015/X2016/X2017/X2018/X2021/X2022/X2023/ |
| | | X2024/X2073/X249/X253/X2827/X2828/X2895/X2896/X2899/X2900/X2901/ |
| | | X2902/X2904/X2906/X2908/X2910/X2912/X2913/X2914/X3244/X3865/ |
| | | X3940/X3946/X3949/X3951/X3953/X3954/X3955/X3956/X3958/X3959/X3960/ |
| | | X4315/X485/X487/X5103/X5106/X5107/X5108/X5109/X5111/X5113/X5115/ |
| | | X513/X5499/X6314/X6320/X6322/X6693/X822/X823/X824/X860/X863 |
| ASN_seq_aaDown_N | 2.33 | X1223/X1224/X1225/X1226/X1231/X1240/X1904/X1907/X1908/X1919/X216/ |
| | | X217/X26/X2782/X2799/X393/X424/X426/X427/X7/X744/X748/X749/X751/X752/X92 |
| ASN_seq_aaDown_P | 2.33 | X10462/X10463/X10464/X10465/X10466/X10467/X10468/X10469/X10470/X10471/ |
| | | X10472/X10473/X10474/X10475/X10476/X10477/X10478/X10479/X10480/ |
| | | X10481/X10482/X10483/X10484/X10485/X10486/X10487/X10488/X10489/ |
| | | X10490/X10491/X10492/X10493/X10494/X10495/X10496/X10497/X10498/ |
| | | X10499/X10500/X10501/X10502/X10503/X10504/X10505/X10506/X10507/ |
| | | X10508/X10509/X10510/X10512/X10514/X10517/X10519/X10524/X10549/ |
| | | X10558/X10568/X11108/X11109/X11110/X11111/X11112/X11113/X11114/ |
| | | X11115/X11116/X11117/X11118/X11119/X11120/X11121/X11122/X11123/X11124/ |
| | | X11125/X11126/X11127/X11128/X11129/X11130/X11131/X11132/X11133/ |
| | | X11134/X11135/X11136/X11137/X11138/X11139/X11140/X11142/X11144/ |
| | | X11147/X11163/X11174/X11180/X11584/X11585/X11586/X11587/X11588/ |
| | | X11589/X11590/X11591/X11592/X11593/X11594/X11595/X11596/X11597/ |
| | | X11598/X11599/X11600/X11601/X11602/X11603/X11605/X11618/X11627/ |
| | | X11897/X11898/X11899/X11900/X11901/X11902/X11903/X11904/X11905/ |
| | | X11906/X11916/X12090/X12091/X12092/X12093/X12200/X1356/X1357/X1358/ |
| | | X1359/X1360/X1361/X1362/X1363/X1364/X1365/X1439/X1440/X1441/X1502/ |
| | | X2055/X2056/X2057/X2058/X2059/X2060/X2061/X2062/X2063/X2064/ |
| | | X2065/X2066/X2067/X2068/X2069/X2120/X214/X2161/X2162/X2163/X2164/ |
| | | X2165/X2166/X2243/X2244/X254/X265/X29/X2944/X2945/X2946/X2947/ |
| | | X2948/X2949/X2950/X2951/X2952/X2953/X2954/X2955/X2956/X2957/X2958/ |
| | | X2959/X2960/X2961/X2962/X2996/X2997/X3002/X3004/X3063/X3064/X3065/ |
| | | X3066/X3068/X3069/X3070/X3071/X3072/X3073/X3074/X3075/X3148/ |
| | | X3149/X3150/X3991/X3992/X3993/X3994/X3995/X3996/X3997/X3998/X3999/ |
| | | X4000/X4001/X4002/X4003/X4004/X4005/X4006/X4007/X4008/X4009/X4025/ |
| | | X4026/X4032/X4033/X4037/X4038/X4039/X4040/X4042/X4114/X4115/ |
| | | X4116/X4117/X4118/X4119/X4122/X4123/X4124/X4125/X4126/X4127/X4128/ |
| | | X4129/X4130/X4131/X4132/X4133/X4134/X4136/X4137/X4138/X4188/X4189/ |
| | | X4190/X4191/X4193/X489/X510/X511/X512/X5141/X5142/X5143/X5144/ |
| | | X5145/X5146/X5147/X5148/X5149/X5150/X5151/X5152/X5153/X5154/X5155/ |
| | | X5161/X5164/X5165/X5169/X5170/X5171/X5172/X5173/X5174/X5178/ |
| | | X5179/X5180/X5181/X5261/X5262/X5263/X5264/X5265/X5266/X5267/X5268/ |
| | | X5269/X5270/X5272/X5273/X5274/X5275/X5276/X5277/X5278/X5279/X5280/ |
| | | X5281/X5282/X5283/X5284/X5285/X5286/X5287/X5288/X5289/X5290/ |
| | | X5291/X5292/X5293/X5297/X5298/X5299/X5300/X5301/X5302/X5334/X5335/ |
| | | X5336/X5338/X5340/X5341/X5344/X6344/X6345/X6346/X6347/X6348/X6349/ |
| | | X6350/X6351/X6352/X6353/X6355/X6356/X6357/X6358/X6359/X6363/ |
| | | X6364/X6365/X6366/X6367/X6368/X6369/X6444/X6445/X6446/X6447/X6448/ |
| | | X6449/X6450/X6451/X6452/X6453/X6454/X6455/X6456/X6457/X6458/X6459/ |
| | | X6460/X6461/X6462/X6463/X6465/X6466/X6467/X6468/X6469/X6470/ |
| | | X6471/X6472/X6473/X6474/X6475/X6476/X6477/X6478/X6479/X6480/X6481/ |
| | | X6482/X6483/X6484/X6485/X6486/X6487/X6488/X6489/X6490/X6491/X6495/ |
| | | X6496/X6497/X6498/X6499/X6500/X6501/X6502/X6515/X6517/X6518/ |
| | | X6519/X6522/X6523/X6525/X6528/X6531/X7517/X7518/X7519/X7520/X7521/ |
| | | X7522/X7524/X7525/X7526/X7527/X7528/X7529/X7530/X7590/X7591/X7592/ |
| | | X7593/X7594/X7595/X7596/X7597/X7598/X7599/X7600/X7601/X7602/ |
| | | X7603/X7604/X7605/X7606/X7607/X7608/X7609/X7610/X7611/X7612/X7613/ |
| | | X7614/X7615/X7616/X7617/X7618/X7619/X7620/X7621/X7623/X7625/X7626/ |
| | | X7627/X7628/X7629/X7630/X7631/X7632/X7633/X7634/X7635/X7636/ |
| | | X7637/X7638/X7639/X7640/X7641/X7642/X7643/X7644/X7645/X7646/X7647/ |
| | | X7648/X7649/X7650/X7651/X7652/X7654/X7655/X7656/X7657/X7658/X7659/ |
| | | X7660/X7661/X7666/X7668/X7669/X7672/X7673/X7676/X7677/X7683/ |
| | | X7689/X84/X854/X855/X856/X857/X858/X859/X8633/X8634/X8635/X8636/ |
| | | X8676/X8677/X8678/X8679/X8680/X8681/X8682/X8683/X8684/X8685/X8686/ |
| | | X8687/X8688/X8689/X8690/X8691/X8692/X8693/X8694/X8695/X8696/X8697/ |
| | | X8698/X8699/X8700/X8701/X8702/X8703/X8704/X8705/X8706/X8707/ |
| | | X8708/X8709/X8710/X8711/X8712/X8713/X8714/X8715/X8716/X8717/X8718/ |
| | | X8719/X8720/X8721/X8722/X8723/X8724/X8725/X8726/X8727/X8728/X8729/ |
| | | X8730/X8731/X8732/X8733/X8734/X8735/X8736/X8737/X8738/X8739/ |
| | | X8740/X8741/X8742/X8743/X8744/X8745/X8746/X8747/X8748/X8749/X8751/ |
| | | X8754/X8755/X8760/X8763/X8764/X8766/X8772/X8811/X90/X907/X9638/ |
| | | X9639/X9640/X9641/X9642/X9643/X9644/X9645/X9646/X9647/X9648/X9649/ |
| | | X9650/X9651/X9652/X9653/X9654/X9655/X9656/X9657/X9658/X9659/X9660/ |
| | | X9661/X9662/X9663/X9664/X9665/X9666/X9667/X9668/X9669/X9670/ |
| | | X9671/X9672/X9673/X9674/X9675/X9676/X9677/X9678/X9679/X9680/X9681/ |
| | | X9682/X9683/X9684/X9685/X9686/X9687/X9688/X9689/X9690/X9691/X9692/ |
| | | X9693/X9694/X9695/X9696/X9697/X9698/X9699/X9700/X9701/X9702/ |
| | | X9703/X9705/X9707/X9710/X9713/X9714/X9716/X9720/X9750/X9765 |
| ASN_seq_aaDown_Q | 2.33 | X10494/X1356/X1357/X1359/X1360/X2055/X2057/X2058/X2059/X2061/X2063/ |
| | | X2120/X214/X2944/X2945/X2946/X2947/X2949/X2951/X2953/X2954/X2957/ |
| | | X2996/X2997/X3002/X3004/X3991/X3992/X3993/X3995/X3996/X3998/ |
| | | X3999/X4002/X4004/X4006/X4025/X4026/X4032/X4033/X4037/X4038/X4039/ |
| | | X4040/X4042/X510/X5141/X5142/X5144/X5145/X5146/X5147/X5149/X5151/ |
| | | X5154/X5161/X5164/X5165/X5169/X5170/X5171/X5172/X5173/X5174/X5178/ |
| | | X5179/X5180/X5181/X5293/X6344/X6345/X6346/X6348/X6349/X6352/ |
| | | X6353/X6355/X6356/X6357/X6358/X6359/X6363/X6364/X6365/X6366/X6367/ |
| | | X6368/X6369/X6462/X6490/X6491/X7517/X7519/X7520/X7521/X7522/X7524/ |
| | | X7525/X7526/X7527/X7528/X7529/X7530/X7615/X7618/X7621/X7651/ |
| | | X7652/X84/X855/X856/X8633/X8634/X8635/X8636/X8707/X8709/X8712/X8715/ |
| | | X8716/X8742/X9672/X9674/X9677/X9689 |
| ASN_seq_aaDown_R | 2.33 | X1189/X1194/X1316/X1317/X1318/X1320/X1321/X1322/X1528/X1529/X1867/ |
| | | X1961/X2011/X2012/X2013/X2015/X2016/X2017/X2021/X2022/X2023/X2285/ |
| | | X253/X2743/X2855/X2895/X2896/X2899/X2900/X2901/X2904/X2910/X2913/ |
| | | X2914/X3907/X3940/X3946/X3953/X3954/X3955/X3958/X3959/X400/ |
| | | X485/X487/X5103/X5106/X5107/X5108/X568/X6314/X718/X822/X823/X824/X965/X966 |
| ASN_seq_aaDown_S | 2.33 | X1993/X2885 |
| ASN_seq_aaDown_T | 2.33 | X117/X13/X1354/X263/X45/X508/X509/X851/X852 |
| ASN_seq_aaDown_V | 2.33 | X1294/X1295/X1296/X170/X1991/X1995/X1996/X245/X27/X2882/X2887/X3936/ |
| | | X470/X80/X801/X802 |
| ASN_seq_aaDown_W | 2.33 | X10428/X10494/X10496/X10499/X10501/X10503/X10505/X10508/X10510/X11134/ |
| | | X11137/X11139/X11602/X1174/X1181/X1188/X1189/X1190/X1191/X1192/ |
| | | X1193/X1194/X1356/X1358/X1359/X1361/X1362/X1363/X1364/X1365/ |
| | | X1441/X16/X183/X1849/X1865/X1866/X1867/X1868/X1869/X1894/X193/X194/ |
| | | X195/X196/X2055/X2056/X2057/X2060/X2062/X2063/X2064/X2065/X2066/ |
| | | X2067/X2068/X2069/X2120/X2164/X2165/X23/X265/X2741/X2742/X2743/ |
| | | X2763/X29/X2944/X2946/X2948/X2950/X2951/X2952/X2953/X2955/X2956/ |
| | | X2957/X2958/X2959/X2960/X2961/X2962/X2996/X2997/X3002/X3004/X3068/ |
| | | X3069/X3071/X3072/X3075/X3775/X3802/X386/X3935/X396/X397/X398/ |
| | | X399/X3992/X3993/X3994/X3995/X3996/X3997/X3998/X400/X4000/X4001/ |
| | | X4002/X4003/X4005/X4006/X4007/X4008/X4009/X401/X402/X4025/X4026/ |
| | | X4032/X4033/X4037/X4038/X4039/X4040/X4042/X4122/X4123/X4126/X4128/ |
| | | X4132/X4136/X4137/X4138/X5098/X511/X512/X5141/X5142/X5143/X5144/ |
| | | X5145/X5147/X5148/X5150/X5151/X5152/X5153/X5154/X5155/X5161/ |
| | | X5164/X5165/X5169/X5170/X5171/X5172/X5173/X5174/X5178/X5179/X5180/ |
| | | X5181/X5272/X5274/X5278/X5281/X5282/X5289/X5291/X5293/X5297/X5298/ |
| | | X5299/X5300/X5301/X5302/X6191/X6344/X6346/X6347/X6348/X6349/ |
| | | X6350/X6351/X6352/X6353/X6355/X6356/X6357/X6358/X6359/X6363/X6364/ |
| | | X6365/X6366/X6367/X6368/X6369/X6460/X6461/X6462/X6470/X6472/X6474/ |
| | | X6478/X6486/X6487/X6490/X6491/X6495/X6496/X6497/X6498/X6499/ |
| | | X6500/X6501/X6502/X67/X698/X707/X713/X714/X715/X716/X717/X718/X719/ |
| | | X7401/X75/X7517/X7518/X7519/X7520/X7521/X7522/X7524/X7525/X7526/ |
| | | X7527/X7528/X7529/X7530/X76/X7614/X7615/X7618/X7620/X7621/X7631/ |
| | | X7632/X7639/X7641/X7644/X7647/X7649/X7651/X7652/X7654/X7655/ |
| | | X7656/X7657/X7658/X7659/X7660/X7661/X83/X854/X855/X8558/X8560/X857/ |
| | | X858/X859/X8633/X8634/X8635/X8636/X8707/X8709/X8712/X8714/X8715/ |
| | | X8716/X8718/X8721/X8724/X8726/X8733/X8734/X8737/X8740/X8742/X8743/ |
| | | X8744/X8745/X8746/X8747/X8748/X90/X9575/X9672/X9674/X9677/X9680/ |
| | | X9681/X9684/X9687/X9689/X9691/X9694/X9696/X9698/X9700/X9701/X9702 |
| ASN_seq_aaDown_Y | 2.33 | X1274/X1971/X2857/X3865/X49 |
| ASN_seq_aaUp_C | 2.33 | X238 |
| ASN_seq_aaUp_E | 2.33 | X10462/X10463/X10464/X10465/X10466/X10467/X10468/X10469/X10470/X10471/ |
| | | X10472/X10473/X10474/X10475/X10476/X10477/X10478/X10479/X10480/ |
| | | X10481/X10482/X10483/X10484/X10485/X10486/X10487/X10488/X10489/ |
| | | X10490/X10491/X10492/X10493/X10494/X10495/X10496/X10497/X10498/ |
| | | X10499/X10500/X10501/X10502/X10503/X10504/X10505/X10506/X10507/ |
| | | X10508/X10509/X10510/X10512/X10514/X10517/X10519/X10524/X10549/ |
| | | X10558/X10568/X11108/X11109/X11110/X11111/X11112/X11113/X11114/ |
| | | X11115/X11116/X11117/X11118/X11119/X11120/X11121/X11122/X11123/X11124/ |
| | | X11125/X11126/X11127/X11128/X11129/X11130/X11131/X11132/X11133/ |
| | | X11134/X11135/X11136/X11137/X11138/X11139/X11140/X11142/X11144/ |
| | | X11147/X11163/X11174/X11180/X113/X11584/X11585/X11586/X11587/ |
| | | X11588/X11589/X11590/X11591/X11592/X11593/X11594/X11595/X11596/ |
| | | X11597/X11598/X11599/X11600/X11601/X11602/X11603/X11605/X11618/X11627/ |
| | | X11897/X11898/X11899/X11900/X11901/X11902/X11903/X11904/X11905/ |
| | | X11906/X11916/X1206/X1207/X1208/X12090/X12091/X12092/X12093/ |
| | | X12200/X1266/X1267/X1316/X1317/X1318/X1320/X1321/X1322/X1356/X1359/ |
| | | X1439/X1440/X1441/X1883/X1884/X1952/X1953/X198/X2011/X2012/X2013/ |
| | | X2015/X2016/X2017/X2018/X2021/X2022/X2023/X2024/X2055/X2057/ |
| | | X2063/X2120/X2161/X2162/X2163/X2164/X2165/X2166/X2243/X25/X253/ |
| | | X254/X2756/X2827/X2828/X2895/X2896/X2899/X2900/X2901/X2902/X2904/ |
| | | X2906/X2908/X2910/X2912/X2913/X2914/X2944/X2946/X2951/X2953/X2957/ |
| | | X2996/X2997/X3002/X3004/X3063/X3064/X3065/X3066/X3068/X3069/X3070/ |
| | | X3071/X3072/X3073/X3074/X3075/X3148/X3244/X3435/X3865/X3940/ |
| | | X3946/X3947/X3949/X3951/X3953/X3954/X3955/X3956/X3958/X3959/X3960/ |
| | | X3992/X3993/X3995/X3996/X3998/X4002/X4006/X4025/X4026/X4032/X4033/ |
| | | X4037/X4038/X4039/X4040/X4042/X406/X407/X4114/X4115/X4116/X4117/ |
| | | X4118/X4119/X4122/X4123/X4124/X4125/X4126/X4127/X4128/X4129/ |
| | | X4130/X4131/X4132/X4133/X4134/X4136/X4137/X4138/X4188/X4190/X4315/ |
| | | X4316/X4544/X485/X487/X489/X5103/X5106/X5107/X5108/X5109/X5111/ |
| | | X5113/X5115/X5141/X5142/X5144/X5145/X5147/X5151/X5154/X5161/X5164/ |
| | | X5165/X5169/X5170/X5171/X5172/X5173/X5174/X5178/X5179/X5180/ |
| | | X5181/X5261/X5262/X5263/X5264/X5265/X5266/X5267/X5268/X5269/X5270/ |
| | | X5272/X5273/X5274/X5275/X5276/X5277/X5278/X5279/X5280/X5281/X5282/ |
| | | X5283/X5284/X5285/X5286/X5287/X5288/X5289/X5290/X5291/X5292/ |
| | | X5293/X5297/X5298/X5299/X5300/X5301/X5302/X5334/X5336/X5338/X5498/ |
| | | X5499/X5756/X6314/X6320/X6322/X6344/X6346/X6348/X6349/X6352/X6353/ |
| | | X6355/X6356/X6357/X6358/X6359/X6363/X6364/X6365/X6366/X6367/ |
| | | X6368/X6369/X6444/X6445/X6446/X6447/X6448/X6449/X6450/X6451/X6452/ |
| | | X6453/X6454/X6455/X6456/X6457/X6458/X6459/X6460/X6461/X6462/X6463/ |
| | | X6465/X6466/X6467/X6468/X6469/X6470/X6471/X6472/X6473/X6474/ |
| | | X6475/X6476/X6477/X6478/X6479/X6480/X6481/X6482/X6483/X6484/X6485/ |
| | | X6486/X6487/X6488/X6489/X6490/X6491/X6495/X6496/X6497/X6498/X6499/ |
| | | X6500/X6501/X6502/X6515/X6517/X6518/X6519/X6523/X6693/X6694/ |
| | | X6972/X727/X728/X729/X7517/X7519/X7520/X7521/X7522/X7524/X7525/X7526/ |
| | | X7527/X7528/X7529/X7530/X7590/X7591/X7592/X7593/X7594/X7595/ |
| | | X7596/X7597/X7598/X7599/X7600/X7601/X7602/X7603/X7604/X7605/X7606/ |
| | | X7607/X7608/X7609/X7610/X7611/X7612/X7613/X7614/X7615/X7616/X7617/ |
| | | X7618/X7619/X7620/X7621/X7623/X7625/X7626/X7627/X7628/X7629/ |
| | | X7630/X7631/X7632/X7633/X7634/X7635/X7636/X7637/X7638/X7639/X7640/ |
| | | X7641/X7642/X7643/X7644/X7645/X7646/X7647/X7648/X7649/X7650/X7651/ |
| | | X7652/X7654/X7655/X7656/X7657/X7658/X7659/X7660/X7661/X7666/ |
| | | X7668/X7669/X7673/X7676/X7677/X78/X7847/X822/X823/X824/X855/X8633/ |
| | | X8634/X8635/X8636/X8676/X8677/X8678/X8679/X8680/X8681/X8682/X8683/ |
| | | X8684/X8685/X8686/X8687/X8688/X8689/X8690/X8691/X8692/X8693/ |
| | | X8694/X8695/X8696/X8697/X8698/X8699/X8700/X8701/X8702/X8703/X8704/ |
| | | X8705/X8706/X8707/X8708/X8709/X8710/X8711/X8712/X8713/X8714/X8715/ |
| | | X8716/X8717/X8718/X8719/X8720/X8721/X8722/X8723/X8724/X8725/ |
| | | X8726/X8727/X8728/X8729/X8730/X8731/X8732/X8733/X8734/X8735/X8736/ |
| | | X8737/X8738/X8739/X8740/X8741/X8742/X8743/X8744/X8745/X8746/X8747/ |
| | | X8748/X8749/X8751/X8754/X8755/X8760/X8764/X8766/X8811/X907/ |
| | | X9638/X9639/X9640/X9641/X9642/X9643/X9644/X9645/X9646/X9647/X9648/ |
| | | X9649/X9650/X9651/X9652/X9653/X9654/X9655/X9656/X9657/X9658/X9659/ |
| | | X9660/X9661/X9662/X9663/X9664/X9665/X9666/X9667/X9668/X9669/ |
| | | X9670/X9671/X9672/X9673/X9674/X9675/X9676/X9677/X9678/X9679/X9680/ |
| | | X9681/X9682/X9683/X9684/X9685/X9686/X9687/X9688/X9689/X9690/X9691/ |
| | | X9692/X9693/X9694/X9695/X9696/X9697/X9698/X9699/X9700/X9701/ |
| | | X9702/X9703/X9705/X9707/X9710/X9714/X9716/X9720/X9750/X9765 |
| ASN_seq_aaUp_F | 2.33 | X1970/X2843/X2851/X3885/X3892/X5054 |
| ASN_seq_aaUp_H | 2.33 | X115/X12/X1350/X1351/X1352/X170/X2050/X2051/X2287/X260/X2941/X3226/ |
| | | X4295/X44/X504/X846/X847 |
| ASN_seq_aaUp_I | 2.33 | X113 |
| ASN_seq_aaUp_K | 2.33 | X1195/X1198/X1870/X1873/X1875/X2745/X2747/X2761/X3790/X3801/X4967/X720 |
| ASN_seq_aaUp_L | 2.33 | X1294/X1295/X1369/X1430/X1961/X1996/X2071/X2143/X245/X27/X2855/X3030/ |
| | | X3907/X3909/X470/X5073/X80/X801/X802/X861 |
| ASN_seq_aaUp_N | 2.33 | X10494/X10496/X10499/X10501/X10503/X10505/X10508/X10510/X11134/X11137/ |
| | | X11139/X11602/X1356/X1357/X1358/X1359/X1360/X1361/X1362/X1363/ |
| | | X1364/X1365/X1441/X2055/X2056/X2057/X2058/X2059/X2060/X2061/ |
| | | X2062/X2063/X2065/X2066/X2067/X2068/X2069/X2120/X214/X2164/X2165/ |
| | | X265/X29/X2944/X2945/X2946/X2947/X2948/X2949/X2950/X2951/X2952/ |
| | | X2953/X2954/X2955/X2956/X2957/X2958/X2960/X2961/X2962/X2996/X2997/ |
| | | X3002/X3004/X3068/X3069/X3071/X3075/X3991/X3992/X3993/X3994/X3995/ |
| | | X3996/X3997/X3998/X3999/X4000/X4001/X4002/X4003/X4004/X4005/ |
| | | X4006/X4008/X4009/X4025/X4026/X4032/X4033/X4037/X4038/X4039/X4040/ |
| | | X4042/X4122/X4126/X4128/X4136/X4137/X4138/X510/X511/X512/X5141/ |
| | | X5142/X5143/X5144/X5145/X5146/X5147/X5149/X5150/X5151/X5152/X5153/ |
| | | X5154/X5155/X5161/X5164/X5165/X5169/X5170/X5171/X5172/X5173/X5174/ |
| | | X5178/X5179/X5180/X5181/X5272/X5274/X5281/X5291/X5293/X5297/ |
| | | X5298/X5299/X5300/X5301/X5302/X6344/X6345/X6346/X6347/X6348/X6349/ |
| | | X6350/X6351/X6352/X6353/X6355/X6356/X6357/X6358/X6359/X6363/X6364/ |
| | | X6365/X6366/X6367/X6368/X6369/X6460/X6462/X6472/X6474/X6486/ |
| | | X6487/X6490/X6491/X6495/X6496/X6497/X6498/X6499/X6500/X6501/X6502/ |
| | | X7517/X7518/X7519/X7520/X7521/X7522/X7524/X7525/X7526/X7527/X7528/ |
| | | X7529/X7530/X7614/X7615/X7618/X7621/X7631/X7632/X7641/X7644/ |
| | | X7647/X7649/X7651/X7652/X7654/X7655/X7656/X7657/X7658/X7659/X7660/ |
| | | X7661/X84/X854/X855/X856/X857/X858/X859/X8633/X8634/X8635/X8636/ |
| | | X8707/X8709/X8712/X8715/X8716/X8718/X8721/X8724/X8726/X8733/X8734/ |
| | | X8737/X8740/X8742/X8743/X8744/X8745/X8746/X8747/X8748/X90/X9672/ |
| | | X9674/X9677/X9680/X9681/X9684/X9687/X9689/X9691/X9694/X9696/ |
| | | X9698/X9700/X9701/X9702 |
| ASN_seq_aaUp_P | 2.33 | X1316/X1317/X1318/X1320/X1321/X2011/X2012/X2013/X2015/X2017/X2018/ |
| | | X2021/X2023/X2024/X253/X2895/X2899/X2901/X2902/X2904/X2906/X2908/ |
| | | X2910/X2912/X2913/X3244/X3435/X3946/X3949/X3951/X3953/X3955/X3956/ |
| | | X3958/X3960/X4315/X4316/X4544/X485/X487/X5106/X5108/X5109/X5111/ |
| | | X5113/X5115/X5498/X5499/X5756/X6320/X6322/X6693/X6694/X6972/ |
| | | X7847/X822/X823/X824 |
| ASN_seq_aaUp_Q | 2.33 | X1527/X1528/X1529/X2283/X2285/X3207/X568/X965/X966 |
| ASN_seq_aaUp_R | 2.33 | X113/X1344/X1345/X1346/X1347/X170/X2042/X2043/X2044/X2045/X258/X2932/ |
| | | X2933/X2934/X393/X3980/X3981/X502/X843/X844 |
| ASN_seq_aaUp_S | 2.33 | X1316/X1317/X1318/X1320/X1321/X1322/X2011/X2012/X2013/X2015/X2016/ |
| | | X2017/X2018/X2021/X2022/X2023/X238/X253/X2895/X2896/X2899/X2900/ |
| | | X2901/X2902/X2904/X2910/X2913/X2914/X3940/X3946/X3953/X3954/X3955/ |
| | | X3956/X3958/X3959/X485/X487/X5103/X5106/X5107/X5108/X5109/X6314/ |
| | | X822/X823/X824 |
| ASN_seq_aaUp_T | 2.33 | X1195/X1198/X1866/X1870/X1873/X1875/X2742/X2745/X2747/X2761/X3790/ |
| | | X3799/X3801/X3935/X4966/X4967/X5098/X7073/X720 |
| ASN_seq_aaUp_V | 2.33 | X1195/X1198/X1294/X1295/X1296/X1870/X1873/X1875/X1880/X1991/X1995/ |
| | | X1996/X245/X27/X2745/X2747/X2753/X2761/X2882/X2887/X3790/X3794/ |
| | | X3801/X3936/X470/X4967/X720/X80/X801/X802 |
| ASN_seq_aaUp_W | 2.33 | X105/X1274/X1277/X1281/X1283/X1284/X1287/X1950/X1961/X1971/X1974/ |
| | | X1978/X1980/X2049/X239/X241/X2825/X2855/X2857/X2868/X2940/X3863/ |
| | | X3907/X3909/X3987/X461/X462/X464/X5073/X5470/X787/X789/X792/X793/X795 |
| ASN_seq_RSA_accproe | 2.33 | X105/X1191/X1193/X1283/X1284/X1869/X1894/X193/X194/X195/X196/X1980/ |
| | | X23/X241/X2763/X3802/X397/X399/X401/X402/X461/X464/X49/X714/X715/ |
| | | X717/X719/X75/X76/X789/X792/X795/X83 |
| ASN_seq_SS_sspro8C | 2.33 | X1221/X1222/X1223/X1224/X1225/X1226/X1228/X1229/X1230/X1231/X1232/ |
| | | X1233/X1234/X1235/X1236/X1237/X1238/X1239/X1240/X1277/X1281/X1287/ |
| | | X1294/X1295/X1431/X1432/X1433/X1435/X1437/X188/X1895/X1898/X1901/ |
| | | X1902/X1903/X1904/X1905/X1906/X1907/X1908/X1910/X1911/X1912/ |
| | | X1913/X1914/X1915/X1916/X1917/X1918/X1919/X1920/X1921/X1922/X1923/ |
| | | X1924/X1925/X1961/X1974/X1978/X1996/X2035/X2036/X2145/X2146/X2148/ |
| | | X2149/X215/X2150/X2151/X2152/X2154/X2157/X216/X217/X218/X239/ |
| | | X245/X26/X27/X2766/X2768/X2769/X2770/X2773/X2774/X2775/X2776/X2777/ |
| | | X2779/X2780/X2781/X2782/X2783/X2785/X2786/X2788/X2789/X2790/ |
| | | X2791/X2792/X2793/X2794/X2795/X2796/X2797/X2798/X2799/X2855/X2868/ |
| | | X2922/X2923/X2924/X2926/X2927/X2928/X3032/X3033/X3034/X3035/X3036/ |
| | | X3038/X3039/X3040/X3042/X3044/X3045/X3046/X3050/X3051/X3052/ |
| | | X3053/X3059/X3060/X3062/X32/X3805/X3811/X3813/X3814/X3815/X3816/ |
| | | X3817/X3819/X3820/X3822/X3823/X3825/X3826/X3827/X3828/X3829/X3830/ |
| | | X3831/X3832/X3834/X3835/X3836/X3907/X393/X3966/X3968/X3970/X3972/ |
| | | X3973/X3974/X3976/X3977/X3978/X4066/X4067/X4069/X4070/X4071/ |
| | | X4072/X4073/X4074/X4076/X4078/X4079/X4080/X4081/X4082/X4083/X4087/ |
| | | X4088/X4089/X4090/X4093/X4095/X4097/X4098/X4099/X4102/X4103/X4105/ |
| | | X4108/X4109/X4111/X4112/X424/X425/X426/X427/X428/X429/X430/X462/ |
| | | X470/X4974/X4976/X4978/X4979/X4980/X4981/X4984/X4985/X4987/X4988/ |
| | | X4989/X4990/X4991/X5120/X5123/X5125/X5127/X5128/X5129/X5131/ |
| | | X5133/X5134/X5135/X5200/X5201/X5203/X5204/X5205/X5206/X5208/X5210/ |
| | | X5211/X5212/X5213/X5214/X5217/X5220/X5221/X5222/X5225/X5227/X5229/ |
| | | X5230/X5231/X5232/X5233/X5237/X5238/X5239/X5242/X5243/X5244/ |
| | | X5245/X5248/X5249/X5252/X5253/X5254/X5255/X5259/X532/X6201/X6205/ |
| | | X6207/X6208/X6211/X6325/X6328/X6331/X6334/X6335/X6336/X6338/X6339/ |
| | | X6381/X6383/X6384/X6387/X6391/X6394/X6395/X6397/X6399/X6400/ |
| | | X6401/X6406/X6407/X6409/X6413/X6414/X6415/X6418/X6419/X6420/X6422/ |
| | | X6424/X6426/X6427/X6428/X6430/X6431/X6432/X6433/X6438/X69/X7/X744/ |
| | | X746/X747/X748/X749/X750/X751/X7510/X7513/X7514/X7515/X752/X753/ |
| | | X754/X7541/X755/X7550/X7551/X7556/X7557/X756/X7560/X7563/X7564/ |
| | | X7568/X7570/X7571/X7572/X7575/X7576/X7577/X7578/X787/X793/X80/ |
| | | X801/X802/X8631/X8654/X8655/X8660/X8661/X8664/X901/X903/X92/X93/X94/X9630 |
| ASN_seq_SS_sspro8E | 2.33 | X117/X1188/X1190/X1192/X1195/X1198/X13/X1354/X1479/X1865/X1866/X1868/ |
| | | X1870/X1873/X1875/X1894/X2212/X263/X2741/X2742/X2745/X2747/ |
| | | X2761/X2763/X3790/X3799/X3801/X3802/X3935/X398/X45/X4966/X4967/X508/ |
| | | X509/X5098/X7073/X713/X716/X720/X8213/X851/X852 |
| ASN_seq_SS_sspro8H | 2.33 | X10462/X10463/X10465/X10466/X10469/X10470/X10471/X10472/X10473/X10474/ |
| | | X10475/X10477/X10478/X10479/X10480/X10481/X10483/X10484/X10485/ |
| | | X10486/X10487/X10488/X10489/X10491/X10492/X10493/X10494/X10495/ |
| | | X10496/X10497/X10498/X10499/X10500/X10501/X10502/X10503/X10504/ |
| | | X10505/X10506/X10507/X10508/X10509/X10510/X10512/X10514/X10517/ |
| | | X10519/X10524/X11108/X11109/X11112/X11114/X11117/X11118/X11119/ |
| | | X11120/X11121/X11122/X11123/X11125/X11126/X11127/X11128/X11129/ |
| | | X11130/X11131/X11132/X11134/X11135/X11136/X11137/X11138/X11139/X11140/ |
| | | X11142/X11144/X11147/X11585/X11587/X11590/X11593/X11594/X11595/ |
| | | X11596/X11597/X11599/X11600/X11601/X11602/X11603/X11605/X11898/ |
| | | X11901/X11904/X11905/X11906/X12091/X1356/X1357/X1358/X1359/X1360/ |
| | | X1361/X1362/X1363/X1364/X1365/X1439/X1440/X1441/X2055/X2056/ |
| | | X2057/X2058/X2059/X2060/X2061/X2062/X2063/X2064/X2065/X2066/X2067/ |
| | | X2068/X2069/X2120/X214/X2161/X2162/X2163/X2164/X2165/X2166/X254/ |
| | | X265/X29/X2944/X2945/X2946/X2947/X2948/X2949/X2950/X2951/X2952/ |
| | | X2953/X2954/X2955/X2956/X2957/X2958/X2959/X2960/X2961/X2962/X2996/ |
| | | X2997/X3002/X3004/X3063/X3064/X3065/X3066/X3068/X3069/X3070/ |
| | | X3071/X3072/X3073/X3074/X3075/X3991/X3992/X3993/X3994/X3995/X3996/ |
| | | X3997/X3998/X3999/X4000/X4001/X4002/X4003/X4004/X4005/X4006/X4007/ |
| | | X4008/X4009/X4025/X4026/X4032/X4033/X4037/X4038/X4039/X4040/ |
| | | X4042/X4114/X4115/X4116/X4117/X4118/X4119/X4122/X4123/X4124/X4125/ |
| | | X4126/X4127/X4128/X4129/X4130/X4131/X4132/X4133/X4134/X4136/X4137/ |
| | | X4138/X489/X510/X511/X512/X5141/X5142/X5143/X5144/X5145/X5146/ |
| | | X5147/X5148/X5149/X5150/X5151/X5152/X5153/X5154/X5155/X5161/X5164/ |
| | | X5165/X5169/X5170/X5171/X5172/X5173/X5174/X5178/X5179/X5180/ |
| | | X5181/X5262/X5263/X5264/X5265/X5266/X5267/X5268/X5269/X5270/X5272/ |
| | | X5273/X5274/X5275/X5276/X5277/X5278/X5279/X5280/X5281/X5282/X5283/ |
| | | X5284/X5285/X5286/X5287/X5288/X5289/X5290/X5291/X5292/X5293/ |
| | | X5297/X5298/X5299/X5300/X5301/X5302/X6344/X6345/X6346/X6347/X6348/ |
| | | X6349/X6350/X6351/X6352/X6353/X6355/X6356/X6357/X6358/X6359/X6363/ |
| | | X6364/X6365/X6366/X6367/X6368/X6369/X6445/X6446/X6447/X6448/ |
| | | X6449/X6450/X6451/X6452/X6453/X6454/X6455/X6456/X6457/X6458/X6459/ |
| | | X6460/X6461/X6462/X6463/X6465/X6466/X6467/X6468/X6469/X6470/X6471/ |
| | | X6472/X6473/X6474/X6475/X6476/X6477/X6478/X6479/X6480/X6481/ |
| | | X6482/X6483/X6484/X6485/X6486/X6487/X6488/X6489/X6490/X6491/X6495/ |
| | | X6496/X6497/X6498/X6499/X6500/X6501/X6502/X6519/X7517/X7518/X7519/ |
| | | X7520/X7521/X7522/X7524/X7525/X7526/X7527/X7528/X7529/X7530/ |
| | | X7591/X7592/X7593/X7594/X7595/X7596/X7597/X7598/X7599/X7600/X7601/ |
| | | X7602/X7603/X7604/X7605/X7606/X7607/X7608/X7609/X7610/X7611/X7612/ |
| | | X7614/X7615/X7616/X7617/X7618/X7619/X7620/X7621/X7623/X7625/ |
| | | X7626/X7627/X7628/X7629/X7630/X7631/X7632/X7633/X7634/X7635/X7636/ |
| | | X7637/X7638/X7639/X7640/X7641/X7642/X7643/X7644/X7645/X7646/X7647/ |
| | | X7648/X7649/X7650/X7651/X7652/X7654/X7655/X7656/X7657/X7658/ |
| | | X7659/X7660/X7661/X7666/X7669/X7677/X84/X854/X855/X856/X857/X858/ |
| | | X859/X8633/X8634/X8635/X8636/X8676/X8678/X8679/X8680/X8681/X8682/ |
| | | X8683/X8684/X8685/X8686/X8687/X8688/X8689/X8690/X8692/X8693/X8694/ |
| | | X8695/X8696/X8697/X8698/X8699/X8700/X8701/X8702/X8703/X8704/ |
| | | X8705/X8707/X8708/X8709/X8710/X8711/X8712/X8713/X8714/X8715/X8716/ |
| | | X8717/X8718/X8719/X8720/X8721/X8722/X8723/X8724/X8725/X8726/X8727/ |
| | | X8728/X8729/X8730/X8731/X8732/X8733/X8734/X8735/X8736/X8737/ |
| | | X8738/X8739/X8740/X8741/X8742/X8743/X8744/X8745/X8746/X8747/X8748/ |
| | | X8749/X8751/X8755/X8760/X8766/X90/X907/X9639/X9640/X9641/X9643/ |
| | | X9644/X9645/X9646/X9647/X9648/X9649/X9650/X9651/X9652/X9653/X9654/ |
| | | X9655/X9656/X9658/X9659/X9660/X9661/X9662/X9663/X9664/X9666/X9667/ |
| | | X9668/X9669/X9670/X9672/X9673/X9674/X9675/X9676/X9677/X9678/ |
| | | X9679/X9680/X9681/X9682/X9683/X9684/X9685/X9686/X9687/X9688/X9689/ |
| | | X9690/X9691/X9692/X9693/X9694/X9695/X9696/X9697/X9698/X9699/X9700/ |
| | | X9701/X9702/X9703/X9705/X9707/X9710/X9716/X9720 |
| ASN_seq_SS_sspro8S | 2.33 | X1199/X1202/X1204/X1206/X1207/X1208/X1266/X1267/X1316/X1317/X1318/ |
| | | X1320/X1321/X1322/X1344/X1345/X1346/X1347/X1416/X1876/X1878/X1880/ |
| | | X1881/X1883/X1884/X1950/X1951/X1952/X1953/X197/X198/X2011/X2012/ |
| | | X2013/X2015/X2016/X2017/X2018/X2021/X2022/X2023/X2024/X2042/ |
| | | X2043/X2044/X2045/X2123/X24/X25/X253/X258/X2748/X2751/X2753/X2754/ |
| | | X2756/X2825/X2826/X2827/X2828/X2895/X2896/X2899/X2900/X2901/X2902/ |
| | | X2904/X2906/X2908/X2910/X2912/X2913/X2914/X2932/X2933/X2934/ |
| | | X3006/X3244/X3435/X3791/X3794/X3795/X3863/X3864/X3865/X3940/X3946/ |
| | | X3947/X3949/X3951/X3953/X3954/X3955/X3956/X3958/X3959/X3960/X3980/ |
| | | X3981/X403/X406/X407/X4315/X4316/X4544/X485/X487/X4961/X502/ |
| | | X5103/X5106/X5107/X5108/X5109/X5111/X5113/X5115/X5318/X5385/X5386/ |
| | | X5498/X5499/X5756/X6314/X6320/X6322/X6693/X6694/X6895/X6972/X722/ |
| | | X725/X727/X728/X729/X77/X78/X7847/X822/X823/X824/X843/X844 |
| ASN_seq_SS_sspro8T | 2.33 | X115/X12/X1350/X1351/X1352/X2050/X2051/X2287/X260/X2941/X3226/X4295/ |
| | | X44/X504/X846/X847 |
| ASN_seq_SS_ssproE | 2.33 | X117/X1195/X1198/X13/X1354/X1355/X1479/X1870/X1873/X1875/X2054/X2212/ |
| | | X263/X2745/X2747/X3790/X45/X508/X509/X720/X851/X852/X853 |
| ASN_seq_SS_ssproH | 2.33 | X10462/X10463/X10465/X10466/X10469/X10470/X10471/X10472/X10473/X10474/ |
| | | X10475/X10477/X10478/X10479/X10480/X10481/X10483/X10484/X10485/ |
| | | X10486/X10487/X10488/X10489/X10491/X10492/X10493/X10494/X10495/ |
| | | X10496/X10497/X10498/X10499/X10500/X10501/X10502/X10503/X10504/ |
| | | X10505/X10506/X10507/X10508/X10509/X10510/X10512/X10514/X10517/ |
| | | X10519/X10524/X11108/X11109/X11112/X11114/X11117/X11118/X11119/ |
| | | X11120/X11121/X11122/X11123/X11125/X11126/X11127/X11128/X11129/ |
| | | X11130/X11131/X11132/X11134/X11135/X11136/X11137/X11138/X11139/X11140/ |
| | | X11142/X11144/X11147/X11585/X11587/X11590/X11593/X11594/X11595/ |
| | | X11596/X11597/X11599/X11600/X11601/X11602/X11603/X11605/X11898/ |
| | | X11901/X11904/X11905/X11906/X12091/X1356/X1357/X1358/X1359/X1360/ |
| | | X1361/X1362/X1363/X1364/X1365/X1439/X1440/X1441/X2055/X2056/ |
| | | X2057/X2058/X2059/X2060/X2061/X2062/X2063/X2064/X2065/X2066/X2067/ |
| | | X2068/X2069/X2120/X214/X2161/X2162/X2163/X2164/X2165/X2166/X254/ |
| | | X265/X29/X2944/X2945/X2946/X2947/X2948/X2949/X2950/X2951/X2952/ |
| | | X2953/X2954/X2955/X2956/X2957/X2958/X2959/X2960/X2961/X2962/X2996/ |
| | | X2997/X3002/X3004/X3063/X3064/X3065/X3066/X3068/X3069/X3070/ |
| | | X3071/X3072/X3073/X3074/X3075/X3991/X3992/X3993/X3994/X3995/X3996/ |
| | | X3997/X3998/X3999/X4000/X4001/X4002/X4003/X4004/X4005/X4006/X4007/ |
| | | X4008/X4009/X4025/X4026/X4032/X4033/X4037/X4038/X4039/X4040/ |
| | | X4042/X4114/X4115/X4116/X4117/X4118/X4119/X4122/X4123/X4124/X4125/ |
| | | X4126/X4127/X4128/X4129/X4130/X4131/X4132/X4133/X4134/X4136/X4137/ |
| | | X4138/X489/X510/X511/X512/X5141/X5142/X5143/X5144/X5145/X5146/ |
| | | X5147/X5148/X5149/X5150/X5151/X5152/X5153/X5154/X5155/X5161/X5164/ |
| | | X5165/X5169/X5170/X5171/X5172/X5173/X5174/X5178/X5179/X5180/ |
| | | X5181/X5262/X5263/X5264/X5265/X5266/X5267/X5268/X5269/X5270/X5272/ |
| | | X5273/X5274/X5275/X5276/X5277/X5278/X5279/X5280/X5281/X5282/X5283/ |
| | | X5284/X5285/X5286/X5287/X5288/X5289/X5290/X5291/X5292/X5293/ |
| | | X5297/X5298/X5299/X5300/X5301/X5302/X6344/X6345/X6346/X6347/X6348/ |
| | | X6349/X6350/X6351/X6352/X6353/X6355/X6356/X6357/X6358/X6359/X6363/ |
| | | X6364/X6365/X6366/X6367/X6368/X6369/X6445/X6446/X6447/X6448/ |
| | | X6449/X6450/X6451/X6452/X6453/X6454/X6455/X6456/X6457/X6458/X6459/ |
| | | X6460/X6461/X6462/X6463/X6465/X6466/X6467/X6468/X6469/X6470/X6471/ |
| | | X6472/X6473/X6474/X6475/X6476/X6477/X6478/X6479/X6480/X6481/ |
| | | X6482/X6483/X6484/X6485/X6486/X6487/X6488/X6489/X6490/X6491/X6495/ |
| | | X6496/X6497/X6498/X6499/X6500/X6501/X6502/X6519/X7517/X7518/X7519/ |
| | | X7520/X7521/X7522/X7524/X7525/X7526/X7527/X7528/X7529/X7530/ |
| | | X7591/X7592/X7593/X7594/X7595/X7596/X7597/X7598/X7599/X7600/X7601/ |
| | | X7602/X7603/X7604/X7605/X7606/X7607/X7608/X7609/X7610/X7611/X7612/ |
| | | X7614/X7615/X7616/X7617/X7618/X7619/X7620/X7621/X7623/X7625/ |
| | | X7626/X7627/X7628/X7629/X7630/X7631/X7632/X7633/X7634/X7635/X7636/ |
| | | X7637/X7638/X7639/X7640/X7641/X7642/X7643/X7644/X7645/X7646/X7647/ |
| | | X7648/X7649/X7650/X7651/X7652/X7654/X7655/X7656/X7657/X7658/ |
| | | X7659/X7660/X7661/X7666/X7669/X7677/X84/X854/X855/X856/X857/X858/ |
| | | X859/X8633/X8634/X8635/X8636/X8676/X8678/X8679/X8680/X8681/X8682/ |
| | | X8683/X8684/X8685/X8686/X8687/X8688/X8689/X8690/X8692/X8693/X8694/ |
| | | X8695/X8696/X8697/X8698/X8699/X8700/X8701/X8702/X8703/X8704/ |
| | | X8705/X8707/X8708/X8709/X8710/X8711/X8712/X8713/X8714/X8715/X8716/ |
| | | X8717/X8718/X8719/X8720/X8721/X8722/X8723/X8724/X8725/X8726/X8727/ |
| | | X8728/X8729/X8730/X8731/X8732/X8733/X8734/X8735/X8736/X8737/ |
| | | X8738/X8739/X8740/X8741/X8742/X8743/X8744/X8745/X8746/X8747/X8748/ |
| | | X8749/X8751/X8755/X8760/X8766/X90/X907/X9639/X9640/X9641/X9643/ |
| | | X9644/X9645/X9646/X9647/X9648/X9649/X9650/X9651/X9652/X9653/X9654/ |
| | | X9655/X9656/X9658/X9659/X9660/X9661/X9662/X9663/X9664/X9666/X9667/ |
| | | X9668/X9669/X9670/X9672/X9673/X9674/X9675/X9676/X9677/X9678/ |
| | | X9679/X9680/X9681/X9682/X9683/X9684/X9685/X9686/X9687/X9688/X9689/ |
| | | X9690/X9691/X9692/X9693/X9694/X9695/X9696/X9697/X9698/X9699/X9700/ |
| | | X9701/X9702/X9703/X9705/X9707/X9710/X9716/X9720 |
| ASN_struct_aa_A | 2.33 | X10077/X10113/X10270/X10271/X10272/X10273/X10274/X10275/X10282/X10454/ |
| | | X10455/X10456/X10457/X10458/X10459/X10460/X10461/X10654/X10655/ |
| | | X10656/X10657/X10658/X10659/X10660/X10661/X10662/X10663/X10664/ |
| | | X10665/X10666/X10667/X10668/X10669/X10670/X10671/X10672/X10673/ |
| | | X10674/X10675/X10676/X10677/X10678/X10679/X10680/X10681/X10682/ |
| | | X10683/X10684/X10685/X10686/X10687/X10689/X10690/X10691/X10692/ |
| | | X10693/X10694/X10695/X10696/X10697/X10698/X10699/X10700/X10701/ |
| | | X10702/X10703/X10704/X10705/X10706/X10707/X10708/X10709/X10710/X10711/ |
| | | X10712/X10713/X10715/X10716/X10717/X10718/X10986/X10987/X10988/ |
| | | X11228/X11229/X11230/X11231/X11232/X11233/X11234/X11235/X11236/ |
| | | X11237/X11238/X11240/X11242/X11243/X11244/X11245/X11246/X11247/ |
| | | X11248/X11249/X11250/X11251/X11252/X11253/X11254/X11255/X11256/ |
| | | X11257/X11259/X11260/X11261/X11262/X11263/X11264/X11265/X11266/ |
| | | X11267/X11268/X11269/X11270/X11271/X11272/X11274/X11275/X11276/ |
| | | X11277/X11278/X11279/X11280/X11281/X11282/X11283/X11284/X11285/X11286/ |
| | | X11422/X11535/X11537/X11648/X11649/X11651/X11652/X11653/X11654/ |
| | | X11655/X11656/X11658/X11660/X11661/X11662/X11663/X11664/X11665/ |
| | | X11666/X11668/X11669/X11670/X11671/X11672/X11673/X11674/X11675/ |
| | | X11676/X11677/X11678/X11679/X11680/X11681/X11683/X11684/X11685/ |
| | | X11686/X11927/X11929/X11930/X11932/X11934/X11935/X11936/X11937/ |
| | | X11938/X11939/X11940/X11942/X11943/X11944/X11945/X11946/X12101/ |
| | | X12102/X12104/X12106/X12107/X12108/X1219/X12196/X1220/X1221/X1222/ |
| | | X1223/X1224/X1225/X1226/X1227/X1228/X1229/X1230/X1231/X1232/X1233/ |
| | | X1234/X1235/X1236/X1237/X1238/X1239/X1240/X1431/X1432/X1433/ |
| | | X1435/X1437/X1502/X1557/X188/X1895/X1896/X1897/X1898/X1899/X1900/ |
| | | X1901/X1902/X1903/X1904/X1905/X1906/X1907/X1908/X1909/X1910/X1911/ |
| | | X1912/X1913/X1914/X1915/X1916/X1917/X1918/X1919/X1920/X1921/X1922/ |
| | | X1923/X1924/X1925/X2035/X2036/X2145/X2146/X2148/X2149/X215/ |
| | | X2150/X2151/X2152/X2154/X2157/X216/X2160/X217/X218/X2244/X2331/X2332/ |
| | | X26/X2718/X2764/X2765/X2766/X2767/X2768/X2769/X2770/X2771/X2772/ |
| | | X2773/X2774/X2775/X2776/X2777/X2778/X2779/X2780/X2781/X2782/ |
| | | X2783/X2784/X2785/X2786/X2787/X2788/X2789/X2790/X2791/X2792/X2793/ |
| | | X2794/X2795/X2796/X2797/X2798/X2799/X2922/X2923/X2924/X2925/X2926/ |
| | | X2927/X2928/X3032/X3033/X3034/X3035/X3036/X3038/X3039/X3040/ |
| | | X3042/X3044/X3045/X3046/X3050/X3051/X3052/X3053/X3056/X3059/X3060/ |
| | | X3061/X3062/X3149/X3150/X32/X3285/X3286/X3287/X3293/X3294/X3299/ |
| | | X3332/X3333/X3755/X3803/X3804/X3805/X3806/X3807/X3808/X3809/X3810/ |
| | | X3811/X3812/X3813/X3814/X3815/X3816/X3817/X3818/X3819/X3820/ |
| | | X3821/X3822/X3823/X3824/X3825/X3826/X3827/X3828/X3829/X3830/X3831/ |
| | | X3832/X3833/X3834/X3835/X3836/X3966/X3967/X3968/X3969/X3970/X3971/ |
| | | X3972/X3973/X3974/X3975/X3976/X3977/X3978/X4066/X4067/X4069/ |
| | | X4070/X4071/X4072/X4073/X4074/X4075/X4076/X4078/X4079/X4080/X4081/ |
| | | X4082/X4083/X4087/X4088/X4089/X4090/X4093/X4095/X4096/X4097/X4098/ |
| | | X4099/X4102/X4103/X4104/X4105/X4108/X4109/X4110/X4111/X4112/ |
| | | X4113/X4132/X4158/X4189/X4191/X4193/X424/X425/X426/X427/X428/X429/ |
| | | X430/X4373/X4374/X4376/X4378/X4380/X4384/X4385/X4389/X4391/X4392/ |
| | | X4394/X4397/X4400/X4442/X4443/X4444/X4445/X4446/X4524/X4942/ |
| | | X4968/X4969/X4970/X4971/X4972/X4973/X4974/X4975/X4976/X4977/X4978/ |
| | | X4979/X4980/X4981/X4982/X4983/X4984/X4985/X4986/X4987/X4988/X4989/ |
| | | X4990/X4991/X5119/X5120/X5121/X5122/X5123/X5124/X5125/X5126/ |
| | | X5127/X5128/X5129/X5130/X5131/X5132/X5133/X5134/X5135/X5200/X5201/ |
| | | X5202/X5203/X5204/X5205/X5206/X5207/X5208/X5210/X5211/X5212/X5213/ |
| | | X5214/X5215/X5217/X5218/X5219/X5220/X5221/X5222/X5225/X5227/ |
| | | X5228/X5229/X5230/X5231/X5232/X5233/X5237/X5238/X5239/X5240/X5241/ |
| | | X5242/X5243/X5244/X5245/X5248/X5249/X5250/X5252/X5253/X5254/X5255/ |
| | | X5256/X5257/X5258/X5259/X5260/X5278/X5289/X5309/X5310/X5315/ |
| | | X532/X5335/X5340/X5341/X5344/X5564/X5565/X5566/X5567/X5568/X5570/ |
| | | X5572/X5575/X5577/X5579/X5580/X5583/X5585/X5586/X5588/X5590/X5591/ |
| | | X5594/X5596/X5597/X5598/X5601/X5604/X5609/X5658/X5659/X5660/X5661/ |
| | | X5662/X5663/X5664/X5665/X5666/X5736/X5737/X5738/X5984/X6199/ |
| | | X6200/X6201/X6202/X6203/X6204/X6205/X6206/X6207/X6208/X6209/X6210/ |
| | | X6211/X6324/X6325/X6326/X6327/X6328/X6329/X6330/X6331/X6332/X6333/ |
| | | X6334/X6335/X6336/X6337/X6338/X6339/X6381/X6382/X6383/X6384/ |
| | | X6385/X6386/X6387/X6388/X6389/X6391/X6392/X6393/X6394/X6395/X6396/ |
| | | X6397/X6399/X6400/X6401/X6402/X6403/X6404/X6405/X6406/X6407/X6408/ |
| | | X6409/X6413/X6414/X6415/X6416/X6417/X6418/X6419/X6420/X6422/ |
| | | X6424/X6425/X6426/X6427/X6428/X6430/X6431/X6432/X6433/X6434/X6435/ |
| | | X6436/X6437/X6438/X6439/X6440/X6441/X6442/X6443/X6470/X6478/X6503/ |
| | | X6506/X6507/X6510/X6525/X6528/X6531/X6760/X6761/X6762/X6763/ |
| | | X6764/X6765/X6767/X6769/X6771/X6772/X6773/X6775/X6777/X6779/X6780/ |
| | | X6782/X6784/X6787/X6789/X6790/X6791/X6793/X6795/X6796/X6798/X6799/ |
| | | X6801/X6802/X6803/X6804/X6805/X6806/X6807/X6808/X6809/X6812/ |
| | | X6817/X6820/X6821/X6823/X6826/X6882/X6883/X6884/X6885/X6886/X6888/ |
| | | X6889/X6890/X6891/X6892/X6893/X6954/X6955/X6956/X6960/X7/X7205/ |
| | | X7206/X7207/X7408/X7409/X7410/X7411/X7412/X744/X745/X746/X747/ |
| | | X748/X749/X750/X7506/X7507/X7508/X7509/X751/X7510/X7511/X7512/X7513/ |
| | | X7514/X7515/X7516/X752/X753/X7537/X7538/X7539/X754/X7540/X7541/ |
| | | X7542/X7543/X7544/X7545/X7546/X7547/X7549/X755/X7550/X7551/X7552/ |
| | | X7553/X7554/X7555/X7556/X7557/X7558/X756/X7560/X7561/X7562/X7563/ |
| | | X7564/X7566/X7568/X7569/X7570/X7571/X7572/X7573/X7574/X7575/ |
| | | X7576/X7577/X7578/X7579/X7580/X7581/X7582/X7583/X7584/X7585/X7586/ |
| | | X7587/X7588/X7589/X7620/X7639/X7662/X7664/X7665/X7683/X7689/X7911/ |
| | | X7912/X7913/X7914/X7915/X7916/X7917/X7918/X7920/X7922/X7924/ |
| | | X7925/X7926/X7928/X7930/X7931/X7932/X7933/X7934/X7935/X7936/X7937/ |
| | | X7938/X7939/X7940/X7942/X7945/X7946/X7948/X7949/X7951/X7952/X7953/ |
| | | X7954/X7955/X7956/X7957/X7958/X7959/X7961/X7963/X7964/X7965/ |
| | | X7966/X7967/X7968/X7969/X7970/X7971/X7972/X7973/X7974/X7975/X7976/ |
| | | X7978/X7979/X7981/X7984/X7990/X7992/X8062/X8064/X8065/X8066/X8067/ |
| | | X8068/X8069/X8070/X8122/X8126/X8127/X8131/X8357/X8358/X8359/ |
| | | X8360/X8361/X8362/X8628/X8629/X8630/X8631/X8632/X8640/X8641/X8642/ |
| | | X8643/X8644/X8645/X8646/X8647/X8648/X8649/X8651/X8652/X8653/X8654/ |
| | | X8655/X8656/X8657/X8658/X8659/X8660/X8661/X8662/X8663/X8664/ |
| | | X8665/X8666/X8667/X8668/X8669/X8670/X8671/X8672/X8673/X8674/X8675/ |
| | | X8714/X8772/X8975/X8976/X8977/X8978/X8979/X8980/X8981/X8982/X8983/ |
| | | X8984/X8985/X8987/X8989/X8990/X8991/X8992/X8993/X8994/X8995/ |
| | | X8996/X8997/X8998/X8999/X9000/X9002/X9003/X9004/X9005/X9006/X9007/ |
| | | X9008/X9009/X901/X9010/X9011/X9012/X9014/X9016/X9017/X9018/X9019/ |
| | | X9020/X9021/X9022/X9023/X9024/X9025/X9026/X9027/X9028/X9029/ |
| | | X903/X9030/X9031/X9032/X9033/X9034/X9035/X9036/X9037/X9038/X9039/ |
| | | X9040/X9041/X9042/X9043/X9044/X9045/X9046/X9047/X9048/X9050/X9053/ |
| | | X9064/X9147/X9148/X9149/X9150/X9151/X9195/X9199/X92/X9200/X93/ |
| | | X9389/X9390/X9391/X9392/X9393/X9394/X9395/X94/X9619/X9620/X9621/ |
| | | X9622/X9623/X9624/X9625/X9626/X9627/X9628/X9629/X9630/X9631/X9632/ |
| | | X9633/X9634/X9635/X9636/X9637/X9896/X9897/X9898/X9899/X9900/X9901/ |
| | | X9902/X9903/X9904/X9905/X9906/X9907/X9908/X9909/X9911/X9912/ |
| | | X9913/X9914/X9915/X9916/X9917/X9918/X9919/X9920/X9921/X9922/X9923/ |
| | | X9924/X9925/X9926/X9927/X9928/X9929/X9930/X9931/X9932/X9933/X9934/ |
| | | X9935/X9936/X9937/X9938/X9939/X9940/X9941/X9942/X9943/X9944/ |
| | | X9945/X9946/X9947/X9948/X9949/X9950/X9951/X9952/X9953/X9954/X9955/ |
| | | X9956/X9957/X9958/X9959/X9960/X9961/X9963/X9964/X9965/X9966/X9967/ |
| | | X9968/X9969/X9970/X9971/X9972/X9973 |
| ASN_struct_aa_C | 2.33 | X10454/X10457/X10458/X10459/X10460/X10461/X10656/X10670/X10679/X10695/ |
| | | X10700/X10702/X11249/X11422/X1219/X1220/X1221/X1222/X1223/ |
| | | X1224/X1225/X1226/X1227/X1228/X1229/X1230/X1231/X1232/X1233/X1234/ |
| | | X1235/X1236/X1237/X1238/X1239/X1240/X1431/X1432/X1433/X1435/X1437/ |
| | | X1557/X1895/X1896/X1897/X1898/X1899/X1900/X1901/X1902/X1903/ |
| | | X1904/X1905/X1906/X1907/X1908/X1909/X1910/X1911/X1912/X1913/X1914/ |
| | | X1915/X1916/X1917/X1918/X1919/X1920/X1921/X1922/X1923/X1924/X1925/ |
| | | X2035/X2036/X2145/X2146/X2148/X2149/X215/X2150/X2151/X2152/X2154/ |
| | | X2157/X216/X2160/X217/X218/X2331/X2332/X26/X2764/X2765/X2766/ |
| | | X2767/X2768/X2769/X2770/X2771/X2772/X2773/X2774/X2775/X2776/X2777/ |
| | | X2778/X2779/X2780/X2781/X2782/X2783/X2784/X2785/X2786/X2787/ |
| | | X2788/X2789/X2790/X2791/X2792/X2793/X2794/X2795/X2796/X2797/X2798/ |
| | | X2799/X2922/X2923/X2924/X2925/X2926/X2927/X2928/X3032/X3033/X3034/ |
| | | X3035/X3036/X3038/X3039/X3040/X3042/X3044/X3045/X3046/X3050/ |
| | | X3051/X3052/X3053/X3056/X3059/X3060/X3061/X3062/X32/X3285/X3286/ |
| | | X3287/X3293/X3294/X3299/X3332/X3803/X3804/X3805/X3806/X3807/X3808/ |
| | | X3809/X3810/X3811/X3812/X3813/X3814/X3815/X3816/X3817/X3818/X3819/ |
| | | X3820/X3821/X3822/X3823/X3824/X3825/X3826/X3827/X3828/X3829/ |
| | | X3830/X3831/X3832/X3833/X3834/X3835/X3836/X3966/X3967/X3968/X3969/ |
| | | X3970/X3972/X3973/X3974/X3975/X3976/X3977/X3978/X4066/X4067/X4069/ |
| | | X4070/X4071/X4072/X4073/X4074/X4075/X4076/X4078/X4079/X4080/ |
| | | X4081/X4082/X4083/X4087/X4088/X4089/X4090/X4093/X4095/X4096/X4097/ |
| | | X4098/X4099/X4102/X4103/X4104/X4105/X4108/X4109/X4110/X4111/X4112/ |
| | | X4113/X4158/X424/X425/X426/X427/X428/X429/X430/X4373/X4374/X4376/ |
| | | X4378/X4380/X4384/X4385/X4389/X4391/X4392/X4394/X4397/X4442/ |
| | | X4968/X4969/X4970/X4971/X4972/X4973/X4974/X4975/X4976/X4977/X4978/ |
| | | X4979/X4980/X4981/X4982/X4983/X4984/X4985/X4986/X4987/X4988/X4989/ |
| | | X4990/X4991/X5119/X5120/X5123/X5124/X5125/X5127/X5128/X5129/ |
| | | X5130/X5131/X5132/X5133/X5134/X5135/X5200/X5201/X5202/X5203/X5204/ |
| | | X5205/X5206/X5207/X5208/X5210/X5211/X5212/X5213/X5214/X5215/X5217/ |
| | | X5218/X5219/X5220/X5221/X5222/X5225/X5227/X5228/X5229/X5230/ |
| | | X5231/X5232/X5233/X5237/X5238/X5239/X5240/X5241/X5242/X5243/X5244/ |
| | | X5245/X5248/X5249/X5250/X5252/X5253/X5254/X5255/X5256/X5257/X5258/ |
| | | X5259/X5260/X5309/X5310/X5315/X532/X5564/X5565/X5566/X5567/ |
| | | X5568/X5570/X5572/X5575/X5577/X5579/X5580/X5583/X5585/X5586/X5588/ |
| | | X5590/X5591/X5594/X5596/X5598/X5604/X5659/X6199/X6200/X6201/X6202/ |
| | | X6203/X6204/X6205/X6206/X6207/X6208/X6209/X6210/X6211/X6325/ |
| | | X6328/X6329/X6330/X6331/X6332/X6334/X6335/X6336/X6337/X6338/X6339/ |
| | | X6381/X6382/X6383/X6384/X6385/X6386/X6387/X6388/X6389/X6391/X6392/ |
| | | X6393/X6394/X6395/X6396/X6397/X6399/X6400/X6401/X6402/X6403/ |
| | | X6404/X6405/X6406/X6407/X6408/X6409/X6413/X6414/X6415/X6416/X6417/ |
| | | X6418/X6419/X6420/X6422/X6424/X6425/X6426/X6427/X6428/X6430/X6431/ |
| | | X6432/X6433/X6434/X6435/X6436/X6437/X6438/X6439/X6440/X6441/ |
| | | X6442/X6443/X6503/X6506/X6507/X6510/X6760/X6761/X6762/X6763/X6764/ |
| | | X6765/X6767/X6769/X6771/X6772/X6773/X6775/X6777/X6780/X6782/X6784/ |
| | | X6787/X6789/X6791/X6793/X6795/X6796/X6798/X6805/X6807/X6812/ |
| | | X7/X7408/X7409/X7411/X7412/X744/X745/X746/X747/X748/X749/X750/X7507/ |
| | | X751/X7510/X7511/X7513/X7514/X7515/X7516/X752/X753/X7537/X7538/ |
| | | X7539/X754/X7540/X7541/X7542/X7543/X7544/X7545/X7546/X7547/X7549/ |
| | | X755/X7550/X7551/X7552/X7553/X7554/X7555/X7556/X7557/X7558/X756/ |
| | | X7560/X7561/X7562/X7563/X7564/X7566/X7568/X7569/X7570/X7571/ |
| | | X7572/X7573/X7574/X7575/X7576/X7577/X7578/X7579/X7580/X7581/X7582/ |
| | | X7583/X7584/X7585/X7586/X7587/X7588/X7589/X7662/X7664/X7665/X7911/ |
| | | X7912/X7913/X7914/X7915/X7916/X7918/X7920/X7922/X7925/X7926/ |
| | | X7928/X7930/X7931/X7936/X7942/X7945/X7946/X7948/X7955/X7957/X7961/ |
| | | X7963/X7965/X7967/X7972/X7974/X7978/X7992/X8631/X8632/X8640/X8641/ |
| | | X8642/X8643/X8644/X8645/X8646/X8647/X8648/X8649/X8651/X8652/ |
| | | X8653/X8654/X8655/X8656/X8657/X8658/X8659/X8660/X8661/X8662/X8663/ |
| | | X8664/X8665/X8666/X8667/X8668/X8669/X8670/X8671/X8672/X8673/X8674/ |
| | | X8675/X8975/X8976/X8977/X8979/X8980/X8985/X8987/X8989/X8990/ |
| | | X8995/X9000/X9003/X9008/X901/X9014/X9016/X9018/X9020/X9025/X9027/ |
| | | X903/X9031/X9036/X9038/X9041/X9043/X9046/X92/X93/X94/X9619/X9620/ |
| | | X9621/X9622/X9623/X9624/X9625/X9627/X9628/X9629/X9630/X9631/X9632/ |
| | | X9633/X9634/X9635/X9636/X9637/X9897/X9898/X9903/X9909/X9912/ |
| | | X9917/X9926/X9932/X9937/X9939/X9943/X9948/X9950/X9967/X9969/X9972 |
| ASN_struct_aa_D | 2.33 | X1529/X188/X2285/X3207 |
| ASN_struct_aa_E | 2.33 | X10462/X10463/X10465/X10469/X10470/X10471/X10472/X10473/X10474/X10483/ |
| | | X10484/X10485/X10495/X11108/X11112/X11117/X11118/X11119/X11585/ |
| | | X1316/X1318/X1344/X1345/X1346/X1347/X1439/X2011/X2013/X2015/ |
| | | X2017/X2018/X2041/X2042/X2043/X2044/X2045/X2161/X2163/X258/X2895/ |
| | | X2899/X2901/X2902/X2904/X2931/X2932/X2933/X2934/X3064/X3066/X3074/ |
| | | X3946/X3953/X3955/X3956/X3979/X3980/X3981/X4116/X4117/X4125/ |
| | | X4129/X4131/X4133/X4134/X485/X502/X5106/X5108/X5109/X5262/X5265/ |
| | | X5267/X5268/X5269/X5270/X5275/X5277/X5279/X5280/X5284/X5285/X5286/ |
| | | X5287/X5288/X5290/X5320/X6445/X6447/X6448/X6449/X6450/X6453/X6454/ |
| | | X6455/X6456/X6457/X6458/X6459/X6465/X6466/X6467/X6468/X6469/ |
| | | X6471/X6475/X6477/X6479/X6480/X6481/X6482/X6483/X6484/X7591/X7594/ |
| | | X7595/X7596/X7597/X7598/X7599/X7600/X7602/X7603/X7604/X7605/X7606/ |
| | | X7607/X7608/X7609/X7610/X7611/X7616/X7619/X7623/X7625/X7626/ |
| | | X7627/X7628/X7629/X7635/X7636/X7637/X7638/X7640/X7643/X822/X843/ |
| | | X844/X8676/X8678/X8680/X8681/X8682/X8683/X8684/X8685/X8686/X8687/ |
| | | X8688/X8689/X8692/X8693/X8694/X8695/X8696/X8697/X8698/X8699/X8700/ |
| | | X8701/X8708/X8710/X8711/X8713/X8717/X8720/X8728/X8729/X8730/X8731/ |
| | | X9639/X9640/X9641/X9643/X9644/X9645/X9646/X9647/X9648/X9649/ |
| | | X9650/X9651/X9652/X9658/X9659/X9660/X9661/X9662/X9663/X9673/X9675/ |
| | | X9676/X9678/X9690 |
| ASN_struct_aa_F | 2.33 | X1316/X1317/X1318/X1320/X1321/X1322/X1366/X1369/X1430/X2011/X2012/ |
| | | X2013/X2015/X2016/X2017/X2018/X2021/X2022/X2023/X2024/X2070/X2071/ |
| | | X2143/X2231/X253/X2895/X2896/X2899/X2900/X2901/X2902/X2904/X2906/ |
| | | X2908/X2910/X2912/X2913/X2914/X3030/X3131/X3133/X3244/X3909/ |
| | | X3940/X3946/X3949/X3951/X3953/X3954/X3955/X3956/X3958/X3959/X3960/ |
| | | X4174/X4315/X485/X487/X5103/X5106/X5107/X5108/X5109/X5111/X5113/ |
| | | X5115/X5499/X6314/X6320/X6322/X6693/X822/X823/X824/X861 |
| ASN_struct_aa_G | 2.33 | X1536/X1538/X1539/X1567/X2297/X2298/X2299/X2301/X2302/X2303/X2343/ |
| | | X3225/X3228/X3229/X3230/X3232/X3233/X3235/X3237/X3238/X3239/X3304/ |
| | | X3305/X3306/X4293/X4294/X4297/X4299/X4300/X4302/X4303/X4304/ |
| | | X4306/X4308/X4310/X4311/X4403/X4404/X4405/X4406/X4408/X5468/X5469/ |
| | | X5471/X5474/X5477/X5480/X5481/X5486/X5492/X5493/X5494/X5615/X5616/ |
| | | X5618/X5619/X5620/X5621/X5623/X570/X6669/X6672/X6673/X6679/X6684/ |
| | | X6686/X6688/X6689/X6833/X6834/X6835/X6836/X6838/X6839/X6841/ |
| | | X7832/X7838/X7843/X7844/X8002/X8003/X8005/X8006/X8008/X8912/X9072/ |
| | | X9073/X9075/X970/X971 |
| ASN_struct_aa_H | 2.33 | X1316/X1317/X1318/X1320/X1321/X1322/X2011/X2012/X2013/X2015/X2016/ |
| | | X2017/X2018/X2021/X2022/X2023/X2024/X253/X2895/X2896/X2899/X2900/ |
| | | X2901/X2902/X2904/X2906/X2908/X2910/X2912/X2913/X2914/X3244/X3940/ |
| | | X3946/X3949/X3951/X3953/X3954/X3955/X3956/X3958/X3959/X3960/ |
| | | X4315/X485/X487/X5103/X5106/X5107/X5108/X5109/X5111/X5113/X5115/ |
| | | X5499/X6314/X6320/X6322/X6693/X822/X823/X824 |
| ASN_struct_aa_I | 2.33 | X10462/X10463/X10464/X10465/X10466/X10467/X10468/X10469/X10470/X10471/ |
| | | X10472/X10473/X10474/X10475/X10476/X10477/X10478/X10479/X10480/ |
| | | X10481/X10482/X10483/X10484/X10485/X10486/X10487/X10488/X10489/ |
| | | X10490/X10491/X10492/X10493/X10494/X10495/X10496/X10497/X10498/ |
| | | X10499/X10500/X10501/X10502/X10503/X10504/X10505/X10506/X10507/ |
| | | X10508/X10509/X10510/X10512/X10514/X10517/X10519/X10524/X10549/ |
| | | X10558/X10568/X11108/X11109/X11110/X11111/X11112/X11113/X11114/ |
| | | X11115/X11116/X11117/X11118/X11119/X11120/X11121/X11122/X11123/X11124/ |
| | | X11125/X11126/X11127/X11128/X11129/X11130/X11131/X11132/X11133/ |
| | | X11134/X11135/X11136/X11137/X11138/X11139/X11140/X11142/X11144/ |
| | | X11147/X11163/X11174/X11180/X11584/X11585/X11586/X11587/X11588/ |
| | | X11589/X11590/X11591/X11592/X11593/X11594/X11595/X11596/X11597/ |
| | | X11598/X11599/X11600/X11601/X11602/X11603/X11605/X11618/X11627/ |
| | | X117/X11897/X11898/X11899/X11900/X11901/X11902/X11903/X11904/X11905/ |
| | | X11906/X11916/X12090/X12091/X12092/X12093/X12200/X13/X1344/ |
| | | X1345/X1346/X1347/X1354/X1355/X1356/X1358/X1359/X1361/X1365/X1439/ |
| | | X1440/X1441/X1479/X1502/X2042/X2043/X2044/X2045/X2054/X2055/X2056/ |
| | | X2057/X2062/X2063/X2066/X2067/X2069/X2120/X2161/X2162/X2163/ |
| | | X2164/X2165/X2166/X2212/X2243/X2244/X254/X258/X263/X2932/X2933/X2934/ |
| | | X2944/X2946/X2950/X2951/X2952/X2953/X2955/X2956/X2957/X2958/ |
| | | X2960/X2962/X2996/X2997/X3002/X3004/X3063/X3064/X3065/X3066/X3068/ |
| | | X3069/X3070/X3071/X3072/X3073/X3074/X3075/X3148/X3149/X3150/X3980/ |
| | | X3981/X3992/X3993/X3994/X3995/X3996/X3997/X3998/X4000/X4001/ |
| | | X4002/X4005/X4006/X4008/X4009/X4025/X4026/X4032/X4033/X4037/X4038/ |
| | | X4039/X4040/X4042/X4114/X4115/X4116/X4117/X4118/X4119/X4122/X4123/ |
| | | X4124/X4125/X4126/X4127/X4128/X4129/X4130/X4131/X4132/X4133/ |
| | | X4134/X4136/X4137/X4138/X4188/X4189/X4190/X4191/X4193/X45/X489/ |
| | | X502/X508/X509/X511/X5141/X5142/X5143/X5144/X5145/X5147/X5150/X5151/ |
| | | X5152/X5153/X5154/X5155/X5161/X5164/X5165/X5169/X5170/X5171/ |
| | | X5172/X5173/X5174/X5178/X5179/X5180/X5181/X5261/X5262/X5263/X5264/ |
| | | X5265/X5266/X5267/X5268/X5269/X5270/X5272/X5273/X5274/X5275/X5276/ |
| | | X5277/X5278/X5279/X5280/X5281/X5282/X5283/X5284/X5285/X5286/ |
| | | X5287/X5288/X5289/X5290/X5291/X5292/X5293/X5297/X5298/X5299/X5300/ |
| | | X5301/X5302/X5334/X5335/X5336/X5338/X5340/X5341/X5344/X6344/X6346/ |
| | | X6347/X6348/X6349/X6350/X6351/X6352/X6353/X6355/X6356/X6357/ |
| | | X6358/X6359/X6363/X6364/X6365/X6366/X6367/X6368/X6369/X6444/X6445/ |
| | | X6446/X6447/X6448/X6449/X6450/X6451/X6452/X6453/X6454/X6455/X6456/ |
| | | X6457/X6458/X6459/X6460/X6461/X6462/X6463/X6465/X6466/X6467/ |
| | | X6468/X6469/X6470/X6471/X6472/X6473/X6474/X6475/X6476/X6477/X6478/ |
| | | X6479/X6480/X6481/X6482/X6483/X6484/X6485/X6486/X6487/X6488/X6489/ |
| | | X6490/X6491/X6495/X6496/X6497/X6498/X6499/X6500/X6501/X6502/ |
| | | X6515/X6517/X6518/X6519/X6523/X6525/X6528/X6531/X7517/X7518/X7519/ |
| | | X7520/X7521/X7522/X7524/X7525/X7526/X7527/X7528/X7529/X7530/X7590/ |
| | | X7591/X7592/X7593/X7594/X7595/X7596/X7597/X7598/X7599/X7600/ |
| | | X7601/X7602/X7603/X7604/X7605/X7606/X7607/X7608/X7609/X7610/X7611/ |
| | | X7612/X7613/X7614/X7615/X7616/X7617/X7618/X7619/X7620/X7621/X7623/ |
| | | X7625/X7626/X7627/X7628/X7629/X7630/X7631/X7632/X7633/X7634/ |
| | | X7635/X7636/X7637/X7638/X7639/X7640/X7641/X7642/X7643/X7644/X7645/ |
| | | X7646/X7647/X7648/X7649/X7650/X7651/X7652/X7654/X7655/X7656/X7657/ |
| | | X7658/X7659/X7660/X7661/X7666/X7668/X7669/X7673/X7676/X7677/ |
| | | X7683/X7689/X843/X844/X851/X852/X853/X854/X855/X858/X8633/X8634/ |
| | | X8635/X8636/X8676/X8677/X8678/X8679/X8680/X8681/X8682/X8683/X8684/ |
| | | X8685/X8686/X8687/X8688/X8689/X8690/X8691/X8692/X8693/X8694/X8695/ |
| | | X8696/X8697/X8698/X8699/X8700/X8701/X8702/X8703/X8704/X8705/ |
| | | X8706/X8707/X8708/X8709/X8710/X8711/X8712/X8713/X8714/X8715/X8716/ |
| | | X8717/X8718/X8719/X8720/X8721/X8722/X8723/X8724/X8725/X8726/X8727/ |
| | | X8728/X8729/X8730/X8731/X8732/X8733/X8734/X8735/X8736/X8737/ |
| | | X8738/X8739/X8740/X8741/X8742/X8743/X8744/X8745/X8746/X8747/X8748/ |
| | | X8749/X8751/X8754/X8755/X8760/X8764/X8766/X8772/X8811/X907/X9638/ |
| | | X9639/X9640/X9641/X9642/X9643/X9644/X9645/X9646/X9647/X9648/X9649/ |
| | | X9650/X9651/X9652/X9653/X9654/X9655/X9656/X9657/X9658/X9659/ |
| | | X9660/X9661/X9662/X9663/X9664/X9665/X9666/X9667/X9668/X9669/X9670/ |
| | | X9671/X9672/X9673/X9674/X9675/X9676/X9677/X9678/X9679/X9680/X9681/ |
| | | X9682/X9683/X9684/X9685/X9686/X9687/X9688/X9689/X9690/X9691/ |
| | | X9692/X9693/X9694/X9695/X9696/X9697/X9698/X9699/X9700/X9701/X9702/ |
| | | X9703/X9705/X9707/X9710/X9714/X9716/X9720/X9750/X9765 |
| ASN_struct_aa_K | 2.33 | X1527/X1528/X1529/X170/X2283/X2285/X568/X965/X966 |
| ASN_struct_aa_L | 2.33 | X1294/X1295/X1296/X1369/X1430/X1995/X1996/X2071/X2140/X2143/X245/ |
| | | X27/X2887/X3030/X470/X80/X801/X802/X861 |
| ASN_struct_aa_M | 2.33 | X1191/X1193/X1366/X1369/X1417/X1430/X1527/X1528/X1529/X1869/X193/ |
| | | X194/X195/X196/X2070/X2071/X2124/X2143/X2231/X2283/X2285/X23/X3007/ |
| | | X3030/X3131/X3133/X3207/X397/X399/X401/X402/X4174/X568/X714/ |
| | | X715/X717/X719/X75/X76/X861/X965/X966 |
| ASN_struct_aa_P | 2.33 | X1536/X1538/X1539/X1567/X1961/X2297/X2298/X2299/X2301/X2302/X2303/ |
| | | X2343/X2855/X3225/X3228/X3229/X3230/X3232/X3233/X3235/X3237/X3238/ |
| | | X3239/X3304/X3305/X3306/X3907/X4293/X4294/X4297/X4299/X4300/ |
| | | X4302/X4303/X4304/X4306/X4308/X4310/X4311/X4403/X4404/X4405/X4406/ |
| | | X4408/X5073/X5468/X5469/X5471/X5474/X5477/X5480/X5481/X5486/X5492/ |
| | | X5493/X5494/X5615/X5616/X5618/X5619/X5620/X5621/X5623/X570/X6669/ |
| | | X6672/X6673/X6679/X6684/X6686/X6688/X6689/X6833/X6834/X6835/ |
| | | X6836/X6838/X6839/X6841/X7832/X7838/X7843/X7844/X8002/X8003/X8005/ |
| | | X8006/X8008/X8912/X9072/X9073/X9075/X970/X971 |
| ASN_struct_aa_R | 2.33 | X1529/X170/X1880/X1881/X2049/X2285/X2753/X2754/X2940/X3207/X3794/ |
| | | X3795/X3987/X5386/X5470 |
| ASN_struct_aa_S | 2.33 | X115/X12/X1274/X1294/X1295/X1296/X1344/X1345/X1346/X1347/X1350/X1351/ |
| | | X1352/X1961/X1971/X1995/X1996/X2041/X2042/X2043/X2044/X2045/ |
| | | X2050/X2051/X2287/X245/X258/X260/X27/X2855/X2857/X2887/X2931/X2932/ |
| | | X2933/X2934/X2941/X3226/X3907/X3979/X3980/X3981/X4295/X44/X470/ |
| | | X502/X504/X5096/X5320/X80/X801/X802/X843/X844/X846/X847 |
| ASN_struct_aa_V | 2.33 | X1880/X2753/X3153/X3794/X3799/X4196/X4966/X5349/X7073/X7088 |
| ASN_struct_aa_W | 2.33 | X115/X12/X1277/X1281/X1287/X1350/X1351/X1352/X1961/X1974/X1978/X2050/ |
| | | X2051/X2287/X239/X260/X2855/X2868/X2941/X3226/X3907/X3909/X393/ |
| | | X4295/X44/X462/X504/X5073/X787/X793/X846/X847 |
| ASN_struct_SS_dsspE | 2.33 | X117/X1195/X1198/X13/X1354/X1870/X1873/X1875/X263/X2745/X2747/X2761/ |
| | | X3790/X3799/X3801/X45/X4966/X4967/X508/X509/X7073/X720/X851/X852 |
| ASN_struct_SS_dsspH | 2.33 | X10428/X1356/X1357/X1358/X1359/X1360/X1361/X1362/X1363/X1364/X1365/ |
| | | X1502/X2055/X2056/X2057/X2058/X2059/X2060/X2061/X2062/X2063/X2064/ |
| | | X2065/X2066/X2067/X2068/X2069/X214/X2244/X265/X29/X2944/X2945/ |
| | | X2946/X2947/X2948/X2949/X2950/X2951/X2952/X2953/X2954/X2955/X2956/ |
| | | X2957/X2958/X2959/X2960/X2961/X2962/X3149/X3150/X3991/X3992/ |
| | | X3993/X3994/X3995/X3996/X3997/X3998/X3999/X4000/X4001/X4002/X4003/ |
| | | X4004/X4005/X4006/X4007/X4008/X4009/X4189/X4191/X4193/X510/X511/ |
| | | X512/X5141/X5142/X5143/X5144/X5145/X5146/X5147/X5148/X5149/X5150/ |
| | | X5151/X5152/X5153/X5154/X5155/X5335/X5340/X5341/X5344/X6191/ |
| | | X6344/X6345/X6346/X6347/X6348/X6349/X6350/X6351/X6352/X6522/X6525/ |
| | | X6528/X6531/X7401/X7517/X7518/X7519/X7520/X7672/X7683/X7689/X84/ |
| | | X854/X855/X8555/X8556/X8558/X856/X857/X858/X859/X8716/X8763/X8772/ |
| | | X90/X9575/X9713 |
| ASN_struct_SS_dsspS | 2.33 | X1527/X1528/X1529/X170/X2283/X2285/X568/X965/X966 |
| ASN_struct_SS_dsspT | 2.33 | X1950/X2825/X3863/X5318 |
| SER.THR_seq_aaAll_F | 2.33 | X29 |
| SER.THR_struct_aa_A | 2.33 | X16/X207/X208/X271/X415/X418/X589/X85/X86/X95 |
| SER.THR_struct_aa_E | 2.33 | X208/X86 |
| SER.THR_struct_aa_F | 2.33 | X208/X86 |
| SER.THR_struct_SS_dssp_G | 2.33 | X29 |
| ASN_seq_aaAll_C | 3.09 | X1993/X238/X2885/X3938 |
| ASN_seq_aaAll_E | 3.09 | X1316/X1317/X1318/X1320/X1321/X1322/X2011/X2012/X2013/X2015/X2017/ |
| | | X2021/X2022/X2023/X253/X2895/X2896/X2899/X2901/X2904/X2910/X2913/ |
| | | X2914/X3940/X3946/X3953/X3955/X3958/X3959/X485/X487/X5103/X5106/ |
| | | X5108/X6314/X822/X823/X824 |
| ASN_seq_aaAll_H | 3.09 | X1993/X2885/X3938/X5096 |
| ASN_seq_aaAll_L | 3.09 | X1961/X2855/X3907 |
| ASN_seq_aaAll_N | 3.09 | X188/X191 |
| ASN_seq_aaAll_R | 3.09 | X5096 |
| ASN_seq_aaAll_V | 3.09 | X170/X1993/X2885 |
| ASN_seq_aaAll_W | 3.09 | X1991/X2882/X3936 |
| ASN_seq_aaDown_A | 3.09 | X1993/X2885/X3938/X5096 |
| ASN_seq_aaDown_C | 3.09 | X1294/X1295/X1996/X238/X245/X27/X470/X5096/X80/X801/X802 |
| ASN_seq_aaDown_L | 3.09 | X113 |
| ASN_seq_aaDown_R | 3.09 | X1296/X1536/X1538/X1539/X1567/X1991/X1995/X2049/X2297/X2298/X2299/ |
| | | X2301/X2302/X2303/X2343/X2882/X2887/X2940/X3225/X3228/X3229/X3230/ |
| | | X3232/X3233/X3235/X3237/X3238/X3239/X3304/X3305/X3306/X3936/ |
| | | X3987/X4293/X4294/X4297/X4299/X4300/X4302/X4303/X4304/X4306/X4308/ |
| | | X4310/X4311/X4403/X4404/X4405/X4406/X4408/X5468/X5469/X5470/X5471/ |
| | | X5474/X5477/X5480/X5481/X5486/X5492/X5493/X5494/X5615/X5616/ |
| | | X5618/X5619/X5620/X5621/X5623/X570/X6669/X6672/X6673/X6679/X6684/ |
| | | X6686/X6688/X6689/X6833/X6834/X6835/X6836/X6838/X6839/X6841/X7832/ |
| | | X7838/X7843/X7844/X8002/X8003/X8005/X8006/X8008/X8912/X9072/X9073/ |
| | | X9075/X970/X971 |
| ASN_seq_aaDown_S | 3.09 | X170 |
| ASN_seq_aaDown_V | 3.09 | X5096 |
| ASN_seq_aaDown_W | 3.09 | X10462/X10463/X10464/X10465/X10466/X10467/X10468/X10469/X10470/X10471/ |
| | | X10472/X10473/X10474/X10475/X10476/X10477/X10478/X10479/X10480/ |
| | | X10481/X10482/X10483/X10484/X10485/X10486/X10487/X10488/X10489/ |
| | | X10490/X10491/X10492/X10493/X10495/X10497/X10498/X10500/X10502/ |
| | | X10504/X10506/X10507/X10509/X10512/X10514/X10517/X10519/X10524/ |
| | | X10549/X10558/X10568/X11108/X11109/X11110/X11111/X11112/X11113/ |
| | | X11114/X11115/X11116/X11117/X11118/X11119/X11120/X11121/X11122/ |
| | | X11123/X11124/X11125/X11126/X11127/X11128/X11129/X11130/X11131/X11132/ |
| | | X11133/X11135/X11136/X11138/X11140/X11142/X11144/X11147/X11163/ |
| | | X11174/X11180/X11584/X11585/X11586/X11587/X11588/X11589/X11590/ |
| | | X11591/X11592/X11593/X11594/X11595/X11596/X11597/X11598/X11599/ |
| | | X11600/X11601/X11603/X11605/X11618/X11627/X11897/X11898/X11899/ |
| | | X11900/X11901/X11902/X11903/X11904/X11905/X11906/X11916/X12090/ |
| | | X12091/X12092/X12093/X12200/X1357/X1360/X1439/X1440/X15/X2058/X2059/ |
| | | X2061/X214/X2161/X2162/X2163/X2166/X2243/X254/X2945/X2947/X2949/ |
| | | X2954/X3063/X3064/X3065/X3066/X3070/X3073/X3074/X3148/X3991/ |
| | | X3999/X4004/X4114/X4115/X4116/X4117/X4118/X4119/X4124/X4125/X4127/ |
| | | X4129/X4130/X4131/X4133/X4134/X4188/X4190/X489/X5036/X510/X5146/ |
| | | X5149/X5261/X5262/X5263/X5264/X5265/X5266/X5267/X5268/X5269/X5270/ |
| | | X5273/X5275/X5276/X5277/X5279/X5280/X5283/X5284/X5285/X5286/ |
| | | X5287/X5288/X5290/X5292/X5334/X5336/X5338/X6260/X6345/X6444/X6445/ |
| | | X6446/X6447/X6448/X6449/X6450/X6451/X6452/X6453/X6454/X6455/X6456/ |
| | | X6457/X6458/X6459/X6463/X6465/X6466/X6467/X6468/X6469/X6471/ |
| | | X6473/X6475/X6476/X6477/X6479/X6480/X6481/X6482/X6483/X6484/X6485/ |
| | | X6488/X6489/X6515/X6517/X6518/X6519/X6523/X7590/X7591/X7592/X7593/ |
| | | X7594/X7595/X7596/X7597/X7598/X7599/X7600/X7601/X7602/X7603/ |
| | | X7604/X7605/X7606/X7607/X7608/X7609/X7610/X7611/X7612/X7613/X7616/ |
| | | X7617/X7619/X7623/X7625/X7626/X7627/X7628/X7629/X7630/X7633/X7634/ |
| | | X7635/X7636/X7637/X7638/X7640/X7642/X7643/X7645/X7646/X7648/ |
| | | X7650/X7666/X7668/X7669/X7673/X7676/X7677/X84/X856/X8676/X8677/ |
| | | X8678/X8679/X8680/X8681/X8682/X8683/X8684/X8685/X8686/X8687/X8688/ |
| | | X8689/X8690/X8691/X8692/X8693/X8694/X8695/X8696/X8697/X8698/X8699/ |
| | | X8700/X8701/X8702/X8703/X8704/X8705/X8706/X8708/X8710/X8711/ |
| | | X8713/X8717/X8719/X8720/X8722/X8723/X8725/X8727/X8728/X8729/X8730/ |
| | | X8731/X8732/X8735/X8736/X8738/X8739/X8741/X8749/X8751/X8754/X8755/ |
| | | X8760/X8764/X8766/X8811/X907/X9638/X9639/X9640/X9641/X9642/X9643/ |
| | | X9644/X9645/X9646/X9647/X9648/X9649/X9650/X9651/X9652/X9653/ |
| | | X9654/X9655/X9656/X9657/X9658/X9659/X9660/X9661/X9662/X9663/X9664/ |
| | | X9665/X9666/X9667/X9668/X9669/X9670/X9671/X9673/X9675/X9676/X9678/ |
| | | X9679/X9682/X9683/X9685/X9686/X9688/X9690/X9692/X9693/X9695/ |
| | | X9697/X9699/X9703/X9705/X9707/X9710/X9714/X9716/X9720/X9750/X9765 |
| ASN_seq_aaDown_Y | 3.09 | X113/X1344/X1345/X1346/X1347/X15/X2041/X2042/X2043/X2044/X2045/X258/ |
| | | X2931/X2932/X2933/X2934/X3979/X3980/X3981/X502/X5320/X843/X844 |
| ASN_seq_aaUp_A | 3.09 | X170 |
| ASN_seq_aaUp_C | 3.09 | X15 |
| ASN_seq_aaUp_E | 3.09 | X1344/X1345/X1346/X1347/X2041/X2042/X2043/X2044/X2045/X258/X2931/ |
| | | X2932/X2933/X2934/X3979/X3980/X3981/X502/X5320/X843/X844 |
| ASN_seq_aaUp_H | 3.09 | X1294/X1295/X1296/X1991/X1995/X1996/X245/X27/X2882/X2887/X3936/X470/ |
| | | X5096/X80/X801/X802 |
| ASN_seq_aaUp_I | 3.09 | X170 |
| ASN_seq_aaUp_L | 3.09 | X1993/X2885/X3938 |
| ASN_seq_aaUp_S | 3.09 | X15 |
| ASN_seq_aaUp_T | 3.09 | X8213 |
| ASN_seq_aaUp_V | 3.09 | X5096 |
| ASN_seq_aaUp_W | 3.09 | X115/X12/X1350/X1351/X1352/X2050/X2051/X2287/X260/X2941/X3226/X4295/ |
| | | X44/X504/X846/X847 |
| ASN_seq_SS_sspro8C | 3.09 | X191/X1993/X2885/X3938/X74 |
| ASN_seq_SS_sspro8S | 3.09 | X113/X170/X2041/X2122/X2907/X2931/X3005/X3943/X3950/X3979/X4473/ |
| | | X5100/X5112/X5320/X6317/X6321/X7503 |
| ASN_seq_SS_sspro8T | 3.09 | X2049/X2940/X3987/X5470 |
| ASN_struct_aa_K | 3.09 | X3207 |
| ASN_struct_aa_L | 3.09 | X1993/X2885/X3031/X3938 |
| ASN_struct_aa_N | 3.09 | X188/X191 |
| ASN_struct_aa_P | 3.09 | X115/X12/X1294/X1295/X1296/X1350/X1351/X1352/X1991/X1995/X1996/X2049/ |
| | | X2050/X2051/X2287/X245/X260/X27/X2882/X2887/X2940/X2941/X3226/ |
| | | X3936/X3987/X4295/X44/X470/X504/X5470/X80/X801/X802/X846/X847 |
| ASN_struct_aa_Q | 3.09 | X4474 |
| ASN_struct_aa_R | 3.09 | X115/X12/X1350/X1351/X1352/X2050/X2051/X2287/X260/X2941/X3226/X4295/ |
| | | X44/X504/X846/X847 |
| ASN_struct_aa_S | 3.09 | X1993/X2885/X3938 |
| ASN_struct_aa_V | 3.09 | X1195/X1198/X1870/X1873/X1875/X2745/X2747/X2761/X3790/X3801/X4967/ |
| | | X720 |
| ASN_struct_aa_W | 3.09 | X1294/X1295/X1296/X1991/X1995/X1996/X245/X27/X2882/X2887/X3936/X4474/ |
| | | X470/X80/X801/X802 |
| ASN_struct_SS_dsspB | 3.09 | X2703/X3745/X4920 |
| ASN_struct_SS_dsspH | 3.09 | X10462/X10463/X10464/X10465/X10466/X10467/X10468/X10469/X10470/X10471/ |
| | | X10472/X10473/X10474/X10475/X10476/X10477/X10478/X10479/X10480/ |
| | | X10481/X10482/X10483/X10484/X10485/X10486/X10487/X10488/X10489/ |
| | | X10490/X10491/X10492/X10493/X10494/X10495/X10496/X10497/X10498/ |
| | | X10499/X10500/X10501/X10502/X10503/X10504/X10505/X10506/X10507/ |
| | | X10508/X10509/X10510/X10512/X10513/X10514/X10517/X10519/X10523/ |
| | | X10524/X10527/X10529/X10538/X10549/X10558/X10568/X11108/X11109/ |
| | | X11110/X11111/X11112/X11113/X11114/X11115/X11116/X11117/X11118/X11119/ |
| | | X11120/X11121/X11122/X11123/X11124/X11125/X11126/X11127/X11128/ |
| | | X11129/X11130/X11131/X11132/X11133/X11134/X11135/X11136/X11137/ |
| | | X11138/X11139/X11140/X11142/X11144/X11147/X11150/X11163/X11174/ |
| | | X11180/X11584/X11585/X11586/X11587/X11588/X11589/X11590/X11591/ |
| | | X11592/X11593/X11594/X11595/X11596/X11597/X11598/X11599/X11600/ |
| | | X11601/X11602/X11603/X11605/X11618/X11627/X11897/X11898/X11899/ |
| | | X11900/X11901/X11902/X11903/X11904/X11905/X11906/X11916/X12090/X12091/ |
| | | X12092/X12093/X12200/X1439/X1440/X1441/X2120/X2161/X2162/X2163/ |
| | | X2164/X2165/X2166/X2243/X254/X2996/X2997/X3002/X3004/X3063/ |
| | | X3064/X3065/X3066/X3068/X3069/X3070/X3071/X3072/X3073/X3074/X3075/ |
| | | X3148/X3184/X4025/X4026/X4032/X4033/X4037/X4038/X4039/X4040/X4042/ |
| | | X4114/X4115/X4116/X4117/X4118/X4119/X4122/X4123/X4124/X4125/ |
| | | X4126/X4127/X4128/X4129/X4130/X4131/X4132/X4133/X4134/X4136/X4137/ |
| | | X4138/X4188/X4190/X4232/X489/X5161/X5164/X5165/X5169/X5170/X5171/ |
| | | X5172/X5173/X5174/X5178/X5179/X5180/X5181/X5261/X5262/X5263/X5264/ |
| | | X5265/X5266/X5267/X5268/X5269/X5270/X5272/X5273/X5274/X5275/ |
| | | X5276/X5277/X5278/X5279/X5280/X5281/X5282/X5283/X5284/X5285/X5286/ |
| | | X5287/X5288/X5289/X5290/X5291/X5292/X5293/X5297/X5298/X5299/X5300/ |
| | | X5301/X5302/X5334/X5336/X5338/X5345/X5387/X5388/X6353/X6355/ |
| | | X6356/X6357/X6358/X6359/X6363/X6364/X6365/X6366/X6367/X6368/X6369/ |
| | | X6444/X6445/X6446/X6447/X6448/X6449/X6450/X6451/X6452/X6453/X6454/ |
| | | X6455/X6456/X6457/X6458/X6459/X6460/X6461/X6462/X6463/X6465/ |
| | | X6466/X6467/X6468/X6469/X6470/X6471/X6472/X6473/X6474/X6475/X6476/ |
| | | X6477/X6478/X6479/X6480/X6481/X6482/X6483/X6484/X6485/X6486/X6487/ |
| | | X6488/X6489/X6490/X6491/X6495/X6496/X6497/X6498/X6499/X6500/ |
| | | X6501/X6502/X6515/X6517/X6518/X6519/X6523/X6524/X6529/X6534/X6567/ |
| | | X6568/X6569/X7521/X7522/X7524/X7525/X7526/X7527/X7528/X7529/X7530/ |
| | | X7590/X7591/X7592/X7593/X7594/X7595/X7596/X7597/X7598/X7599/ |
| | | X7600/X7601/X7602/X7603/X7604/X7605/X7606/X7607/X7608/X7609/X7610/ |
| | | X7611/X7612/X7613/X7614/X7615/X7616/X7617/X7618/X7619/X7620/X7621/ |
| | | X7623/X7625/X7626/X7627/X7628/X7629/X7630/X7631/X7632/X7633/ |
| | | X7634/X7635/X7636/X7637/X7638/X7639/X7640/X7641/X7642/X7643/X7644/ |
| | | X7645/X7646/X7647/X7648/X7649/X7650/X7651/X7652/X7654/X7655/X7656/ |
| | | X7657/X7658/X7659/X7660/X7661/X7666/X7667/X7668/X7669/X7673/ |
| | | X7674/X7676/X7677/X7681/X7686/X7690/X7692/X7720/X7721/X7722/X7723/ |
| | | X8633/X8634/X8635/X8636/X8676/X8677/X8678/X8679/X8680/X8681/X8682/ |
| | | X8683/X8684/X8685/X8686/X8687/X8688/X8689/X8690/X8691/X8692/ |
| | | X8693/X8694/X8695/X8696/X8697/X8698/X8699/X8700/X8701/X8702/X8703/ |
| | | X8704/X8705/X8706/X8707/X8708/X8709/X8710/X8711/X8712/X8713/X8714/ |
| | | X8715/X8717/X8718/X8719/X8720/X8721/X8722/X8723/X8724/X8725/ |
| | | X8726/X8727/X8728/X8729/X8730/X8731/X8732/X8733/X8734/X8735/X8736/ |
| | | X8737/X8738/X8739/X8740/X8741/X8742/X8743/X8744/X8745/X8746/X8747/ |
| | | X8748/X8749/X8750/X8751/X8754/X8755/X8759/X8760/X8764/X8765/ |
| | | X8766/X8769/X8773/X8775/X8778/X8800/X8801/X8802/X8811/X907/X9638/ |
| | | X9639/X9640/X9641/X9642/X9643/X9644/X9645/X9646/X9647/X9648/X9649/ |
| | | X9650/X9651/X9652/X9653/X9654/X9655/X9656/X9657/X9658/X9659/ |
| | | X9660/X9661/X9662/X9663/X9664/X9665/X9666/X9667/X9668/X9669/X9670/ |
| | | X9671/X9672/X9673/X9674/X9675/X9676/X9677/X9678/X9679/X9680/X9681/ |
| | | X9682/X9683/X9684/X9685/X9686/X9687/X9688/X9689/X9690/X9691/ |
| | | X9692/X9693/X9694/X9695/X9696/X9697/X9698/X9699/X9700/X9701/X9702/ |
| | | X9703/X9705/X9706/X9707/X9710/X9714/X9715/X9716/X9719/X9720/X9724/ |
| | | X9727/X9729/X9744/X9745/X9750/X9765 |
| ASN_struct_SS_dsspS | 3.09 | X3207 |
| ASN_struct_SS_dsspT | 3.09 | X1536/X1538/X1539/X1567/X2297/X2298/X2299/X2301/X2302/X2303/X2343/ |
| | | X3225/X3228/X3229/X3230/X3232/X3233/X3235/X3237/X3238/X3239/X3304/ |
| | | X3305/X3306/X4293/X4294/X4297/X4299/X4300/X4302/X4303/X4304/ |
| | | X4306/X4308/X4310/X4311/X4403/X4404/X4405/X4406/X4408/X5468/X5469/ |
| | | X5471/X5474/X5477/X5480/X5481/X5486/X5492/X5493/X5494/X5615/X5616/ |
| | | X5618/X5619/X5620/X5621/X5623/X570/X6669/X6672/X6673/X6679/X6684/ |
| | | X6686/X6688/X6689/X6833/X6834/X6835/X6836/X6838/X6839/X6841/ |
| | | X7832/X7838/X7843/X7844/X8002/X8003/X8005/X8006/X8008/X8912/X9072/ |
| | | X9073/X9075/X970/X971 |
| SER.THR_seq_aaAll_F | 3.09 | X16/X207/X271/X415/X418/X589/X85/X95 |
| SER.THR_struct_aa_P | 3.09 | X16/X207/X208/X271/X415/X418/X589/X85/X86/X95 |
| SER.THR_struct_SS_dsspG | 3.09 | X16/X207/X271/X415/X418/X589/X85/X95 |
| ASN_seq_aaAll_C | 3.72 | X170 |
| ASN_seq_aaAll_K | 3.72 | X170 |
| ASN_seq_aaAll_N | 3.72 | X74 |
| ASN_seq_aaAll_R | 3.72 | X1993/X2885/X3938 |
| ASN_seq_aaAll_W | 3.72 | X1294/X1295/X1296/X1995/X1996/X245/X27/X2887/X470/X5096/X80/X801/X802 |
| ASN_seq_aaDown_C | 3.72 | X1993/X2885/X3938 |
| ASN_seq_aaDown_K | 3.72 | X170 |
| ASN_seq_aaDown_R | 3.72 | X115/X12/X1294/X1295/X1350/X1351/X1352/X1996/X2050/X2051/X2287/X245/ |
| | | X260/X27/X2941/X3226/X4295/X44/X470/X504/X5096/X80/X801/X802/X846/X847 |
| ASN_seq_aaDown_V | 3.72 | X1993/X2885/X3938 |
| ASN_seq_aaUp_C | 3.72 | X170 |
| ASN_seq_aaUp_H | 3.72 | X1993/X2885/X3938 |
| ASN_seq_aaUp_T | 3.72 | X3153/X4196/X5349/X7088 |
| ASN_seq_aaUp_V | 3.72 | X1993/X2885/X3938 |
| ASN_seq_aaUp_W | 3.72 | X1294/X1295/X1296/X1991/X1995/X1996/X245/X27/X2882/X2887/X3936/X470/ |
| | | X80/X801/X802 |
| ASN_struct_aa_N | 3.72 | X74 |
| ASN_struct_aa_P | 3.72 | X5096 |
| ASN_struct_aa_W | 3.72 | X5096 |
| SER.THR_seq_aaAll_F | 3.72 | X208/X86 |
| SER.THR_seq_aaDown_F | 3.72 | X208/X86 |
| SER.THR_seq_aaUp_F | 3.72 | X208/X86 |
| SER.THR_struct_SS_dsspG | 3.72 | X208/X86 |
| ASN_seq_aaAll_W | 4.26 | X3938 |
| ASN_seq_aaDown_R | 4.26 | X3938 |
| ASN_seq_aaUp_W | 4.26 | X5096 |
| ASN_struct_aa_P | 4.26 | X1993/X2885/X3938 |
| ASN_struct_aa_W | 4.26 | X1993/X2885/X3938 |
| ASN_seq_aaAll_W | 4.75 | X1993/X2885 |
| ASN_seq_aaDown_R | 4.75 | X1993/X2885 |
| ASN_seq_aaUp_W | 4.75 | X1993/X2885/X3938 |
| ASN_seq_aaAll_A | Inf | X356 |
| ASN_seq_aaAll_C | Inf | X1333/X1334/X1335/X1336/X1337/X2028/X2029/X2031/X2032/X2033/X2034/ |
| | | X210/X256/X2916/X2918/X2919/X2920/X2921/X31/X356/X3962/X3964/X3965/ |
| | | X496/X497/X5118/X835/X836/X837/X89/X9/X91 |
| ASN_seq_aaAll_D | Inf | X15 |
| ASN_seq_aaAll_E | Inf | X2018/X2024/X2902/X2906/X2907/X2908/X2912/X3244/X3435/X3943/X3949/ |
| | | X3950/X3951/X3956/X3960/X4315/X4316/X4544/X5100/X5109/X5111/X5112/ |
| | | X5113/X5115/X5498/X5499/X5756/X6317/X6320/X6321/X6322/X6693/ |
| | | X6694/X6972/X7503/X7847 |
| ASN_seq_aaAll_F | Inf | X5036/X6260 |
| ASN_seq_aaAll_H | Inf | X1333/X1334/X1335/X1336/X1337/X2028/X2029/X2031/X2032/X2033/X2034/ |
| | | X210/X256/X2916/X2918/X2919/X2920/X2921/X31/X356/X3962/X3964/X3965/ |
| | | X496/X497/X5118/X835/X836/X837/X89/X9/X91 |
| ASN_seq_aaAll_I | Inf | X356 |
| ASN_seq_aaAll_K | Inf | X356 |
| ASN_seq_aaAll_L | Inf | X113/X1294/X1295/X1296/X1344/X1345/X1346/X1347/X1993/X1995/X1996/ |
| | | X2042/X2043/X2044/X2045/X245/X258/X27/X2885/X2887/X2932/X2933/X2934/ |
| | | X356/X3938/X3980/X3981/X470/X502/X80/X801/X802/X843/X844 |
| ASN_seq_aaAll_P | Inf | X1294/X1295/X1296/X1333/X1334/X1335/X1336/X1337/X157/X1991/X1993/ |
| | | X1995/X1996/X2028/X2029/X2031/X2032/X2033/X2034/X210/X245/X256/ |
| | | X27/X2882/X2885/X2887/X2916/X2918/X2919/X2920/X2921/X31/X3936/X3938/ |
| | | X3962/X3964/X3965/X470/X496/X497/X5096/X5118/X80/X801/X802/X835/ |
| | | X836/X837/X89/X9/X91 |
| ASN_seq_aaAll_R | Inf | X1333/X1334/X1335/X1336/X1337/X2028/X2029/X2031/X2032/X2033/X2034/ |
| | | X210/X256/X2916/X2918/X2919/X2920/X2921/X31/X356/X3962/X3964/X3965/ |
| | | X496/X497/X5118/X835/X836/X837/X89/X9/X91 |
| ASN_seq_aaAll_S | Inf | X1316/X1317/X1318/X1320/X1321/X1322/X2011/X2012/X2013/X2015/X2016/ |
| | | X2017/X2018/X2021/X2022/X2023/X253/X2895/X2896/X2899/X2900/X2901/ |
| | | X2902/X2904/X2910/X2913/X2914/X3940/X3946/X3953/X3954/X3955/X3956/ |
| | | X3958/X3959/X485/X487/X5103/X5106/X5107/X5108/X5109/X6314/X822/ |
| | | X823/X824 |
| ASN_seq_aaAll_T | Inf | X1195/X1198/X1870/X1873/X1875/X2745/X2747/X3790/X720 |
| ASN_seq_aaAll_V | Inf | X1333/X1334/X1335/X1336/X2028/X2029/X2032/X2034/X210/X256/X2916/ |
| | | X2919/X2920/X31/X356/X3962/X496/X497/X835/X836/X837/X89/X9/X91 |
| ASN_seq_aaAll_W | Inf | X1333/X1334/X1335/X1336/X1337/X2028/X2029/X2031/X2032/X2033/X2034/ |
| | | X210/X256/X2916/X2918/X2919/X2920/X2921/X31/X3962/X3964/X3965/ |
| | | X496/X497/X5118/X835/X836/X837/X89/X9/X91 |
| ASN_seq_aaDown_A | Inf | X1333/X1334/X1335/X1336/X1337/X18/X2028/X2029/X2031/X2032/X2033/ |
| | | X2034/X210/X256/X2916/X2918/X2919/X2920/X2921/X31/X3962/X3964/X3965/ |
| | | X496/X497/X5118/X835/X836/X837/X89/X9/X91 |
| ASN_seq_aaDown_C | Inf | X1333/X1334/X1335/X1336/X1337/X2028/X2029/X2031/X2032/X2033/X2034/ |
| | | X210/X256/X2916/X2918/X2919/X2920/X2921/X31/X3962/X3964/X3965/ |
| | | X496/X497/X5118/X835/X836/X837/X89/X9/X91 |
| ASN_seq_aaDown_I | Inf | X356 |
| ASN_seq_aaDown_K | Inf | X356 |
| ASN_seq_aaDown_L | Inf | X356/X4474 |
| ASN_seq_aaDown_R | Inf | X1333/X1334/X1335/X1336/X1337/X2028/X2029/X2031/X2032/X2033/X2034/ |
| | | X210/X256/X2916/X2918/X2919/X2920/X2921/X31/X3962/X3964/X3965/ |
| | | X496/X497/X5118/X835/X836/X837/X89/X9/X91 |
| ASN_seq_aaDown_S | Inf | X356 |
| ASN_seq_aaDown_V | Inf | X1333/X1334/X1335/X1336/X1337/X2028/X2029/X2031/X2032/X2033/X2034/ |
| | | X210/X256/X2916/X2918/X2919/X2920/X2921/X31/X356/X3962/X3964/X3965/ |
| | | X496/X497/X5118/X835/X836/X837/X89/X9/X91 |
| ASN_seq_aaDown_W | Inf | X187/X21/X73 |
| ASN_seq_aaUp_A | Inf | X356 |
| ASN_seq_aaUp_C | Inf | X356 |
| ASN_seq_aaUp_D | Inf | X15/X69 |
| ASN_seq_aaUp_F | Inf | X5036/X6260 |
| ASN_seq_aaUp_H | Inf | X1333/X1334/X1335/X1336/X1337/X2028/X2029/X2031/X2032/X2033/X2034/ |
| | | X210/X256/X2916/X2918/X2919/X2920/X2921/X31/X356/X3962/X3964/X3965/ |
| | | X496/X497/X5118/X835/X836/X837/X89/X9/X91 |
| ASN_seq_aaUp_I | Inf | X356 |
| ASN_seq_aaUp_K | Inf | X188/X69 |
| ASN_seq_aaUp_L | Inf | X1333/X1334/X1335/X1336/X1337/X18/X2028/X2029/X2031/X2032/X2033/ |
| | | X2034/X210/X256/X2916/X2918/X2919/X2920/X2921/X31/X3962/X3964/X3965/ |
| | | X496/X497/X5118/X835/X836/X837/X89/X9/X91 |
| ASN_seq_aaUp_P | Inf | X1294/X1295/X1296/X1333/X1334/X1335/X1336/X1337/X157/X1991/X1993/ |
| | | X1995/X1996/X2028/X2029/X2031/X2032/X2033/X2034/X210/X245/X256/ |
| | | X27/X2882/X2885/X2887/X2916/X2918/X2919/X2920/X2921/X31/X3936/X3938/ |
| | | X3962/X3964/X3965/X470/X496/X497/X5096/X5118/X80/X801/X802/X835/ |
| | | X836/X837/X89/X9/X91 |
| ASN_seq_aaUp_Q | Inf | X188/X69 |
| ASN_seq_aaUp_R | Inf | X356 |
| ASN_seq_aaUp_V | Inf | X1333/X1334/X1335/X1336/X1337/X2028/X2029/X2031/X2032/X2033/X2034/ |
| | | X210/X256/X2916/X2918/X2919/X2920/X2921/X31/X3962/X3964/X3965/ |
| | | X496/X497/X5118/X835/X836/X837/X89/X9/X91 |
| ASN_seq_aaUp_W | Inf | X1333/X1334/X1335/X1336/X1337/X2028/X2029/X2031/X2032/X2033/X2034/ |
| | | X210/X256/X2916/X2918/X2919/X2920/X2921/X31/X393/X3962/X3964/X3965/ |
| | | X496/X497/X5118/X835/X836/X837/X89/X9/X91 |
| ASN_seq_RSA_accproe | Inf | X1189/X1194/X1440/X1866/X1867/X2162/X2166/X254/X2742/X2743/X3065/ |
| | | X3070/X3073/X3935/X400/X4114/X4119/X4124/X4127/X4130/X489/X5098/ |
| | | X5264/X5273/X5276/X5283/X5287/X6452/X6463/X6468/X6476/X6483/X718/ |
| | | X7593/X7617/X7628/X7637/X8711/X8730/X907/X9676 |
| ASN_seq_SS_sspro8C | Inf | X1333/X1334/X1335/X1336/X1337/X2028/X2029/X2031/X2032/X2033/X2034/ |
| | | X210/X256/X2916/X2918/X2919/X2920/X2921/X31/X3962/X3964/X3965/ |
| | | X496/X497/X5118/X835/X836/X837/X89/X9/X91 |
| ASN_seq_SS_sspro8G | Inf | X1416/X1417/X2122/X2123/X2124/X3005/X3006/X3007/X4473 |
| ASN_seq_SS_sspro8S | Inf | X356 |
| ASN_seq_SS_sspro8T | Inf | X1536/X1538/X1539/X1567/X2297/X2298/X2299/X2301/X2302/X2303/X2343/ |
| | | X3225/X3228/X3229/X3230/X3232/X3233/X3235/X3237/X3238/X3239/X3304/ |
| | | X3305/X3306/X4293/X4294/X4297/X4299/X4300/X4302/X4303/X4304/ |
| | | X4306/X4308/X4310/X4311/X4403/X4404/X4405/X4406/X4408/X5468/X5469/ |
| | | X5471/X5474/X5477/X5480/X5481/X5486/X5492/X5493/X5494/X5615/X5616/ |
| | | X5618/X5619/X5620/X5621/X5623/X570/X6669/X6672/X6673/X6679/X6684/ |
| | | X6686/X6688/X6689/X6833/X6834/X6835/X6836/X6838/X6839/X6841/ |
| | | X7832/X7838/X7843/X7844/X8002/X8003/X8005/X8006/X8008/X8912/X9072/ |
| | | X9073/X9075/X970/X971 |
| ASN_struct_aa_A | Inf | X18/X69 |
| ASN_struct_aa_D | Inf | X69 |
| ASN_struct_aa_K | Inf | X356 |
| ASN_struct_aa_L | Inf | X1333/X1334/X1335/X1336/X1337/X2028/X2029/X2031/X2032/X2033/X2034/ |
| | | X210/X256/X2916/X2918/X2919/X2920/X2921/X31/X3962/X3964/X3965/ |
| | | X4474/X496/X497/X5118/X835/X836/X837/X89/X9/X91 |
| ASN_struct_aa_P | Inf | X1333/X1334/X1335/X1336/X1337/X157/X2028/X2029/X2031/X2032/X2033/ |
| | | X2034/X210/X256/X2916/X2918/X2919/X2920/X2921/X31/X3962/X3964/X3965/ |
| | | X496/X497/X5118/X835/X836/X837/X89/X9/X91 |
| ASN_struct_aa_R | Inf | X1294/X1295/X1296/X1333/X1334/X1335/X1336/X1337/X1991/X1993/X1995/ |
| | | X1996/X2028/X2029/X2031/X2032/X2033/X2034/X210/X245/X256/X27/X2882/ |
| | | X2885/X2887/X2916/X2918/X2919/X2920/X2921/X31/X356/X3936/X3938/ |
| | | X3962/X3964/X3965/X470/X496/X497/X5096/X5118/X80/X801/X802/X835/ |
| | | X836/X837/X89/X9/X91 |
| ASN_struct_aa_S | Inf | X1333/X1334/X1335/X1336/X1337/X2028/X2029/X2031/X2032/X2033/X2034/ |
| | | X210/X256/X2916/X2918/X2919/X2920/X2921/X31/X3962/X3964/X3965/ |
| | | X496/X497/X5118/X835/X836/X837/X89/X9/X91 |
| ASN_struct_aa_W | Inf | X1333/X1334/X1335/X1336/X1337/X2028/X2029/X2031/X2032/X2033/X2034/ |
| | | X210/X256/X2916/X2918/X2919/X2920/X2921/X31/X3962/X3964/X3965/ |
| | | X496/X497/X5118/X69/X835/X836/X837/X89/X9/X91 |
| ASN_struct_SS_dsspB | Inf | X1164/X1169/X1172/X1837/X184/X1840/X1843/X2708/X2713/X3762/X385/ |
| | | X696/X703 |
| ASN_struct_SS_dsspH | Inf | X393 |
| ASN_struct_SS_dsspS | Inf | X15/X356 |
| SER.THR_seq_aaAll_P | Inf | X16/X207/X208/X271/X415/X418/X589/X85/X86/X95 |

TABLE 3
Table of representative IMRs determined by generalized estimating equations

| Protein_{—} | LogOR | |
|---|---|---|
| Structure | Extrema | Glycan_Motifs |
| ASN_seq_aaAll_A | −1.28 | X10462/X10463/X10465/X10469/X10470/X10471/X10472/X10473/X10474/ |
| | | X10483/X10484/X10485/X10494/X10495/X10496/X10499/X10501/X10503/ |
| | | X10505/X10508/X10510/X11108/X11112/X11117/X11118/X11119/X11134/ |
| | | X11137/X11139/X11585/X11602/X1188/X1189/X1190/X1191/X1192/X1193/ |
| | | X1194/X1195/X1198/X1356/X1359/X1439/X1441/X1479/X1865/X1866/X1867/ |
| | | X1868/X1869/X1870/X1873/X1875/X1894/X193/X194/X195/X1951/X196/X2055/ |
| | | X2057/X2063/X2120/X2161/X2163/X2164/X2165/X2212/X23/X2741/X2742/ |
| | | X2743/X2745/X2747/X2763/X2826/X2944/X2946/X2951/X2953/X2957/X2996/ |
| | | X2997/X3002/X3004/X3064/X3066/X3068/X3069/X3071/X3074/X3075/X3790/ |
| | | X3802/X3864/X3935/X397/X398/X399/X3992/X3993/X3995/X3996/X3998/X400/ |
| | | X4002/X4006/X401/X402/X4025/X4026/X4032/X4033/X4037/X4038/X4039/X4040/ |
| | | X4042/X4116/X4117/X4122/X4125/X4126/X4128/X4129/X4131/X4133/X4134/ |
| | | X4136/X4137/X4138/X5098/X5141/X5142/X5144/X5145/X5147/X5151/X5154/ |
| | | X5161/X5164/X5165/X5169/X5170/X5171/X5172/X5173/X5174/X5178/X5179/ |
| | | X5180/X5181/X5262/X5265/X5267/X5268/X5269/X5270/X5272/X5274/X5275/ |
| | | X5277/X5279/X5280/X5281/X5284/X5285/X5286/X5287/X5288/X5290/X5291/ |
| | | X5293/X5297/X5298/X5299/X5300/X5301/X5302/X6344/X6346/X6348/X6349/ |
| | | X6352/X6353/X6355/X6356/X6357/X6358/X6359/X6363/X6364/X6365/X6366/ |
| | | X6367/X6368/X6369/X6445/X6447/X6448/X6449/X6450/X6453/X6454/X6455/ |
| | | X6456/X6457/X6458/X6459/X6460/X6462/X6465/X6466/X6467/X6468/X6469/ |
| | | X6471/X6472/X6474/X6475/X6477/X6479/X6480/X6481/X6482/X6483/X6484/ |
| | | X6486/X6487/X6490/X6491/X6495/X6496/X6497/X6498/X6499/X6500/X6501/ |
| | | X6502/X713/X714/X715/X716/X717/X718/X719/X720/X75/X7517/X7519/X7520/ |
| | | X7521/X7522/X7524/X7525/X7526/X7527/X7528/X7529/X7530/X7591/X7594/ |
| | | X7595/X7596/X7597/X7598/X7599/X76/X7600/X7602/X7603/X7604/X7605/ |
| | | X7606/X7607/X7608/X7609/X7610/X7611/X7614/X7615/X7616/X7618/X7619/ |
| | | X7621/X7623/X7625/X7626/X7627/X7628/X7629/X7631/X7632/X7635/X7636/ |
| | | X7637/X7638/X7640/X7641/X7643/X7644/X7647/X7649/X7651/X7652/X7654/ |
| | | X7655/X7656/X7657/X7658/X7659/X7660/X7661/X855/X8560/X8633/X8634/ |
| | | X8635/X8636/X8676/X8678/X8680/X8681/X8682/X8683/X8684/X8685/X8686/ |
| | | X8687/X8688/X8689/X8692/X8693/X8694/X8695/X8696/X8697/X8698/X8699/ |
| | | X8700/X8701/X8707/X8708/X8709/X8710/X8711/X8712/X8713/X8715/X8716/ |
| | | X8717/X8718/X8720/X8721/X8724/X8726/X8728/X8729/X8730/X8731/ |
| | | X8733/X8734/X8737/X8740/X8742/X8743/X8744/X8745/X8746/X8747/X8748/ |
| | | X9639/X9640/X9641/X9643/X9644/X9645/X9646/X9647/X9648/X9649/X9650/ |
| | | X9651/X9652/X9658/X9659/X9660/X9661/X9662/X9663/X9672/X9673/ |
| | | X9674/X9675/X9676/X9677/X9678/X9680/X9681/X9684/X9687/X9689/X9690/ |
| | | X9691/X9694/X9696/X9698/X9700/X9701/X9702 |
| ASN_seq_aaAll_C | −1.28 | X10462/X10463/X10465/X10469/X10470/X10471/X10472/X10473/X10474/ |
| | | X10483/X10484/X10485/X10494/X10495/X11108/X11112/X11117/X11118/ |
| | | X11119/X11585/X1439/X2120/X2161/X2163/X2996/X2997/X3002/X3004/ |
| | | X3064/X3066/X3074/X4025/X4026/X4032/X4033/X4037/X4038/X4039/X4040/X4042/ |
| | | X4116/X4117/X4125/X4129/X4131/X4133/X4134/X5161/X5164/X5165/ |
| | | X5169/X5170/X5171/X5172/X5173/X5174/X5178/X5179/X5180/X5181/X5262/ |
| | | X5265/X5267/X5268/X5269/X5270/X5275/X5277/X5279/X5280/X5284/X5285/ |
| | | X5286/X5287/X5288/X5290/X5293/X6353/X6355/X6356/X6357/X6358/ |
| | | X6359/X6363/X6364/X6365/X6366/X6367/X6368/X6369/X6445/X6447/X6448/ |
| | | X6449/X6450/X6453/X6454/X6455/X6456/X6457/X6458/X6459/X6462/X6465/ |
| | | X6466/X6467/X6468/X6469/X6471/X6475/X6477/X6479/X6480/X6481/ |
| | | X6482/X6483/X6484/X6490/X6491/X7521/X7522/X7524/X7525/X7526/X7527/ |
| | | X7528/X7529/X7530/X7591/X7594/X7595/X7596/X7597/X7598/X7599/X7600/ |
| | | X7602/X7603/X7604/X7605/X7606/X7607/X7608/X7609/X7610/X7611/ |
| | | X7615/X7616/X7618/X7619/X7621/X7623/X7625/X7626/X7627/X7628/X7629/ |
| | | X7635/X7636/X7637/X7638/X7640/X7643/X7651/X7652/X8633/X8634/X8635/ |
| | | X8636/X8676/X8678/X8680/X8681/X8682/X8683/X8684/X8685/X8686/ |
| | | X8687/X8688/X8689/X8692/X8693/X8694/X8695/X8696/X8697/X8698/X8699/ |
| | | X8700/X8701/X8707/X8708/X8709/X8710/X8711/X8712/X8713/X8715/X8717/ |
| | | X8720/X8728/X8729/X8730/X8731/X8742/X9639/X9640/X9641/X9643/ |
| | | X9644/X9645/X9646/X9647/X9648/X9649/X9650/X9651/X9652/X9658/X9659/ |
| | | X9660/X9661/X9662/X9663/X9672/X9673/X9674/X9675/X9676/X9677/X9678/ |
| | | X9689/X9690 |
| ASN_seq_aaAll_D | −1.28 | X1167/X1168/X1170/X1175/X1177/X1180/X1182/X1183/X1185/X1267/X1839/ |
| | | X1848/X185/X1850/X1851/X1853/X1856/X1858/X1860/X1861/X190/X1953/ |
| | | X2725/X2727/X2728/X2732/X2733/X2735/X2828/X3782/X3783/X3865/X387/ |
| | | X389/X391/X395/X699/X701/X702/X706/X709/X71/X711 |
| ASN_seq_aaAll_E | −1.28 | X10462/X10463/X10465/X10469/X10470/X10471/X10472/X10473/X10474/X10483/ |
| | | X10484/X10485/X10494/X10495/X110/X11108/X11112/X11117/X11118/ |
| | | X11119/X11585/X1277/X1281/X1287/X1328/X1329/X1330/X1439/X1974/ |
| | | X1978/X1984/X2083/X2120/X2161/X2163/X239/X255/X2861/X2868/X2996/ |
| | | X2997/X3002/X3004/X3064/X3066/X3074/X3922/X4025/X4026/X4032/X4033/ |
| | | X4037/X4038/X4039/X4040/X4042/X4116/X4117/X4125/X4129/X4131/X4133/ |
| | | X4134/X462/X491/X492/X493/X5161/X5164/X5165/X5169/X5170/X5171/ |
| | | X5172/X5173/X5174/X5178/X5179/X5180/X5181/X5262/X5265/X5267/X5268/ |
| | | X5269/X5270/X5275/X5277/X5279/X5280/X5284/X5285/X5286/X5287/ |
| | | X5288/X5290/X5293/X6353/X6355/X6356/X6357/X6358/X6359/X6363/X6364/ |
| | | X6365/X6366/X6367/X6368/X6369/X6445/X6447/X6448/X6449/X6450/X6453/ |
| | | X6454/X6455/X6456/X6457/X6458/X6459/X6462/X6465/X6466/X6467/ |
| | | X6468/X6469/X6471/X6475/X6477/X6479/X6480/X6481/X6482/X6483/X6484/ |
| | | X6490/X6491/X7521/X7522/X7524/X7525/X7526/X7527/X7528/X7529/X7530/ |
| | | X7591/X7594/X7595/X7596/X7597/X7598/X7599/X7600/X7602/X7603/ |
| | | X7604/X7605/X7606/X7607/X7608/X7609/X7610/X7611/X7615/X7616/X7618/ |
| | | X7619/X7621/X7623/X7625/X7626/X7627/X7628/X7629/X7635/X7636/X7637/ |
| | | X7638/X7640/X7643/X7651/X7652/X787/X793/X829/X83/X830/X831/X832/ |
| | | X8633/X8634/X8635/X8636/X8676/X8678/X8680/X8681/X8682/X8683/ |
| | | X8684/X8685/X8686/X8687/X8688/X8689/X8692/X8693/X8694/X8695/X8696/ |
| | | X8697/X8698/X8699/X8700/X8701/X8707/X8708/X8709/X8710/X8711/X8712/ |
| | | X8713/X8715/X8717/X8720/X8728/X8729/X8730/X8731/X8742/X9639/ |
| | | X9640/X9641/X9643/X9644/X9645/X9646/X9647/X9648/X9649/X9650/X9651/ |
| | | X9652/X9658/X9659/X9660/X9661/X9662/X9663/X9672/X9673/X9674/X9675/ |
| | | X9676/X9677/X9678/X9689/X9690 |
| ASN_seq_aaAll_F | −1.28 | X1186/X170/X49/X799 |
| ASN_seq_aaAll_H | −1.28 | X1206/X1207/X1330/X1883/X198/X25/X406/X407/X493/X727/X728/X729/X78/ |
| | | X832 |
| ASN_seq_aaAll_I | −1.28 | X10462/X10463/X10465/X10469/X10470/X10471/X10472/X10473/X10474/X10483/ |
| | | X10484/X10485/X10494/X10495/X11108/X11112/X11117/X11118/X11119/ |
| | | X11585/X1356/X1359/X1439/X187/X192/X2055/X2057/X2063/X21/X2120/ |
| | | X2161/X2163/X25/X2944/X2946/X2951/X2953/X2957/X2996/X2997/X3002/ |
| | | X3004/X3064/X3066/X3074/X390/X392/X393/X3992/X3993/X3995/X3996/ |
| | | X3998/X4002/X4006/X4025/X4026/X4032/X4033/X4037/X4038/X4039/X4040/ |
| | | X4042/X4116/X4117/X4125/X4129/X4131/X4133/X4134/X5141/X5142/ |
| | | X5144/X5145/X5147/X5151/X5154/X5161/X5164/X5165/X5169/X5170/X5171/ |
| | | X5172/X5173/X5174/X5178/X5179/X5180/X5181/X5262/X5265/X5267/X5268/ |
| | | X5269/X5270/X5275/X5277/X5279/X5280/X5284/X5285/X5286/X5287/ |
| | | X5288/X5290/X5293/X6344/X6346/X6348/X6349/X6352/X6353/X6355/X6356/ |
| | | X6357/X6358/X6359/X6363/X6364/X6365/X6366/X6367/X6368/X6369/X6445/ |
| | | X6447/X6448/X6449/X6450/X6453/X6454/X6455/X6456/X6457/X6458/ |
| | | X6459/X6462/X6465/X6466/X6467/X6468/X6469/X6471/X6475/X6477/X6479/ |
| | | X6480/X6481/X6482/X6483/X6484/X6490/X6491/X708/X73/X7517/X7519/ |
| | | X7520/X7521/X7522/X7524/X7525/X7526/X7527/X7528/X7529/X7530/X7591/ |
| | | X7594/X7595/X7596/X7597/X7598/X7599/X7600/X7602/X7603/X7604/X7605/ |
| | | X7606/X7607/X7608/X7609/X7610/X7611/X7615/X7616/X7618/X7619/ |
| | | X7621/X7623/X7625/X7626/X7627/X7628/X7629/X7635/X7636/X7637/X7638/ |
| | | X7640/X7643/X7651/X7652/X855/X8633/X8634/X8635/X8636/X8676/X8678/ |
| | | X8680/X8681/X8682/X8683/X8684/X8685/X8686/X8687/X8688/X8689/X8692/ |
| | | X8693/X8694/X8695/X8696/X8697/X8698/X8699/X8700/X8701/X8707/ |
| | | X8708/X8709/X8710/X8711/X8712/X8713/X8715/X8716/X8717/X8720/X8728/ |
| | | X8729/X8730/X8731/X8742/X9639/X9640/X9641/X9643/X9644/X9645/X9646/ |
| | | X9647/X9648/X9649/X9650/X9651/X9652/X9658/X9659/X9660/X9661/ |
| | | X9662/X9663/X9672/X9673/X9674/X9675/X9676/X9677/X9678/X9689/X9690 |
| ASN_seq_aaAll_L | −1.28 | X1188/X1190/X1191/X1192/X1195/X1198/X1865/X1866/X1868/X1870/X1873/ |
| | | X1875/X1894/X193/X194/X195/X196/X23/X2741/X2742/X2745/X2747/X2763/ |
| | | X3780/X3790/X3802/X3935/X397/X398/X399/X401/X402/X5098/X713/ |
| | | X714/X715/X716/X717/X720/X75/X76 |
| ASN_seq_aaAll_P | −1.28 | X10462/X10463/X10465/X10469/X10470/X10471/X10472/X10473/X10474/X10483/ |
| | | X10484/X10485/X10494/X10495/X10496/X10499/X10501/X10503/X10505/ |
| | | X10508/X10510/X11108/X11112/X11117/X11118/X11119/X11134/X11137/ |
| | | X11139/X11585/X11602/X1188/X1189/X1190/X1191/X1192/X1193/X1194/ |
| | | X1356/X1359/X1439/X1441/X1865/X1866/X1867/X1868/X1869/X1894/X193/ |
| | | X194/X195/X196/X2055/X2057/X2063/X2120/X2161/X2163/X2164/X2165/ |
| | | X23/X2741/X2742/X2743/X2763/X2944/X2946/X2951/X2953/X2957/X2996/ |
| | | X2997/X3002/X3004/X3064/X3066/X3068/X3069/X3071/X3074/X3075/X3769/ |
| | | X3802/X3935/X397/X398/X399/X3992/X3993/X3995/X3996/X3998/X400/ |
| | | X4002/X4006/X401/X402/X4025/X4026/X4032/X4033/X4037/X4038/X4039/ |
| | | X4040/X4042/X4116/X4117/X4122/X4125/X4126/X4128/X4129/X4130/X4131/ |
| | | X4133/X4134/X4136/X4137/X4138/X4935/X5098/X5141/X5142/X5144/ |
| | | X5145/X5147/X5151/X5154/X5161/X5164/X5165/X5169/X5170/X5171/X5172/ |
| | | X5173/X5174/X5178/X5179/X5180/X5181/X5262/X5265/X5267/X5268/X5269/ |
| | | X5270/X5272/X5274/X5275/X5276/X5277/X5279/X5280/X5281/X5284/ |
| | | X5285/X5286/X5287/X5288/X5290/X5291/X5293/X5297/X5298/X5299/X5300/ |
| | | X5301/X5302/X6344/X6346/X6348/X6349/X6352/X6353/X6355/X6356/X6357/ |
| | | X6358/X6359/X6363/X6364/X6365/X6366/X6367/X6368/X6369/X6445/ |
| | | X6447/X6448/X6449/X6450/X6453/X6454/X6455/X6456/X6457/X6458/X6459/ |
| | | X6460/X6462/X6465/X6466/X6467/X6468/X6469/X6471/X6472/X6474/X6475/ |
| | | X6476/X6477/X6479/X6480/X6481/X6482/X6483/X6484/X6486/X6487/ |
| | | X6490/X6491/X6495/X6496/X6497/X6498/X6499/X6500/X6501/X6502/X713/ |
| | | X714/X715/X716/X717/X718/X719/X75/X7517/X7519/X7520/X7521/X7522/ |
| | | X7524/X7525/X7526/X7527/X7528/X7529/X7530/X7591/X7594/X7595/X7596/ |
| | | X7597/X7598/X7599/X76/X7600/X7602/X7603/X7604/X7605/X7606/X7607/ |
| | | X7608/X7609/X7610/X7611/X7614/X7615/X7616/X7617/X7618/X7619/X7621/ |
| | | X7623/X7625/X7626/X7627/X7628/X7629/X7631/X7632/X7635/X7636/ |
| | | X7637/X7638/X7640/X7641/X7643/X7644/X7647/X7649/X7651/X7652/X7654/ |
| | | X7655/X7656/X7657/X7658/X7659/X7660/X7661/X855/X8633/X8634/X8635/ |
| | | X8636/X8676/X8678/X8680/X8681/X8682/X8683/X8684/X8685/X8686/X8687/ |
| | | X8688/X8689/X8692/X8693/X8694/X8695/X8696/X8697/X8698/X8699/ |
| | | X8700/X8701/X8707/X8708/X8709/X8710/X8711/X8712/X8713/X8715/X8716/ |
| | | X8717/X8718/X8720/X8721/X8724/X8726/X8728/X8729/X8730/X8731/X8733/ |
| | | X8734/X8737/X8740/X8742/X8743/X8744/X8745/X8746/X8747/X8748/ |
| | | X9639/X9640/X9641/X9643/X9644/X9645/X9646/X9647/X9648/X9649/X9650/ |
| | | X9651/X9652/X9658/X9659/X9660/X9661/X9662/X9663/X9672/X9673/X9674/ |
| | | X9675/X9676/X9677/X9678/X9680/X9681/X9684/X9687/X9689/X9690/ |
| | | X9691/X9694/X9696/X9698/X9700/X9701/X9702 |
| ASN_seq_aaAll_Q | −1.28 | X10494/X2120/X2996/X2997/X3002/X3004/X4025/X4026/X4032/X4033/X4037/ |
| | | X4038/X4039/X4040/X4042/X5161/X5164/X5165/X5169/X5170/X5171/X5172/ |
| | | X5173/X5174/X5178/X5179/X5180/X5181/X5293/X6353/X6355/X6356/ |
| | | X6357/X6358/X6359/X6363/X6364/X6365/X6366/X6367/X6368/X6369/X6462/ |
| | | X6490/X6491/X7521/X7522/X7524/X7525/X7526/X7527/X7528/X7529/X7530/ |
| | | X7615/X7618/X7621/X7651/X7652/X8633/X8634/X8635/X8636/X8707/ |
| | | X8709/X8712/X8715/X8742/X9672/X9674/X9677/X9689 |
| ASN_seq_aaAll_R | −1.28 | X189/X388/X394/X70/X700/X705/X7394 |
| ASN_seq_aaAll_S | −1.28 | X117/X1177/X13/X1328/X1354/X190/X2083/X263/X389/X395/X45/X508/X509/ |
| | | X706/X71/X711/X829/X851/X852 |
| ASN_seq_aaAll_V | −1.28 | X1862/X4920 |
| ASN_seq_aaAll_W | −1.28 | X1357/X1360/X2058/X2059/X2061/X214/X2945/X2947/X2949/X2954/X3991/ |
| | | X3999/X4004/X510/X5146/X5149/X6345/X84/X856 |
| ASN_seq_aaDown_A | −1.28 | X1199/X1202/X1206/X1355/X1464/X1876/X197/X198/X2054/X2186/X24/X25/ |
| | | X3117/X403/X406/X407/X722/X725/X727/X728/X77/X78/X853 |
| ASN_seq_aaDown_D | −1.28 | X10494/X2120/X2996/X2997/X3002/X3004/X4025/X4026/X4032/X4033/X4037/ |
| | | X4038/X4039/X4040/X4042/X5161/X5164/X5165/X5169/X5170/X5171/X5172/ |
| | | X5173/X5174/X5178/X5179/X5180/X5181/X5293/X6353/X6355/X6356/ |
| | | X6357/X6358/X6359/X6363/X6364/X6365/X6366/X6367/X6368/X6369/X6462/ |
| | | X6490/X6491/X708/X7521/X7522/X7524/X7525/X7526/X7527/X7528/X7529/ |
| | | X7530/X7615/X7618/X7621/X7651/X7652/X8633/X8634/X8635/X8636/X8707/ |
| | | X8709/X8712/X8715/X8742/X9672/X9674/X9677/X9689 |
| ASN_seq_aaDown_G | −1.28 | X1193/X1832/X193/X194/X195/X196/X23/X2705/X3750/X397/X399/X401/X402/ |
| | | X4928/X714/X715/X719/X75/X76 |
| ASN_seq_aaDown_H | −1.28 | X1328/X1330/X2083/X493/X829/X832 |
| ASN_seq_aaDown_I | −1.28 | X10494/X1356/X1359/X2055/X2057/X2063/X2120/X2944/X2946/X2951/X2953/ |
| | | X2957/X2996/X2997/X3002/X3004/X3992/X3993/X3995/X3996/X3998/X4002/ |
| | | X4006/X4025/X4026/X4032/X4033/X4037/X4038/X4039/X4040/X4042/ |
| | | X5141/X5142/X5144/X5145/X5147/X5151/X5154/X5161/X5164/X5165/X5169/ |
| | | X5170/X5171/X5172/X5173/X5174/X5178/X5179/X5180/X5181/X5293/X6344/ |
| | | X6346/X6348/X6349/X6352/X6353/X6355/X6356/X6357/X6358/X6359/ |
| | | X6363/X6364/X6365/X6366/X6367/X6368/X6369/X6462/X6490/X6491/X69/ |
| | | X7517/X7519/X7520/X7521/X7522/X7524/X7525/X7526/X7527/X7528/X7529/ |
| | | X7530/X7615/X7618/X7621/X7651/X7652/X855/X8633/X8634/X8635/X8636/ |
| | | X8707/X8709/X8712/X8715/X8716/X8742/X9672/X9674/X9677/X9689 |
| ASN_seq_aaDown_L | −1.28 | X1188/X1190/X1192/X1206/X1207/X1266/X1865/X1868/X187/X1883/X1894/ |
| | | X192/X1952/X198/X21/X25/X2741/X2763/X2827/X3802/X390/X392/X398/ |
| | | X406/X407/X708/X713/X716/X727/X728/X729/X73/X78/X83 |
| ASN_seq_aaDown_M | −1.28 | X1206/X198/X25/X406/X407/X727/X728/X78 |
| ASN_seq_aaDown_P | −1.28 | X10494/X1356/X1357/X1359/X1360/X1440/X2055/X2057/X2058/X2059/X2061/ |
| | | X2063/X2120/X214/X2162/X2166/X254/X2944/X2945/X2946/X2947/X2949/ |
| | | X2951/X2953/X2954/X2957/X2996/X2997/X3002/X3004/X3065/X3070/X3073/ |
| | | X3750/X3991/X3992/X3993/X3995/X3996/X3998/X3999/X4002/X4004/ |
| | | X4006/X4025/X4026/X4032/X4033/X4037/X4038/X4039/X4040/X4042/X4114/ |
| | | X4119/X4124/X4127/X4130/X489/X4928/X510/X5141/X5142/X5144/X5145/ |
| | | X5146/X5147/X5149/X5151/X5154/X5161/X5164/X5165/X5169/X5170/X5171/ |
| | | X5172/X5173/X5174/X5178/X5179/X5180/X5181/X5264/X5273/X5276/ |
| | | X5283/X5287/X5293/X6344/X6345/X6346/X6348/X6349/X6352/X6353/X6355/ |
| | | X6356/X6357/X6358/X6359/X6363/X6364/X6365/X6366/X6367/X6368/X6369/ |
| | | X6452/X6462/X6463/X6468/X6476/X6483/X6490/X6491/X7517/X7519/ |
| | | X7520/X7521/X7522/X7524/X7525/X7526/X7527/X7528/X7529/X7530/X7593/ |
| | | X7615/X7617/X7618/X7621/X7628/X7637/X7651/X7652/X84/X855/X856/ |
| | | X8633/X8634/X8635/X8636/X8707/X8709/X8711/X8712/X8715/X8716/X8730/ |
| | | X8742/X907/X9672/X9674/X9676/X9677/X9689 |
| ASN_seq_aaDown_Q | −1.28 | X104/X1357/X1360/X1832/X2058/X2059/X2061/X214/X234/X235/X2705/X2945/ |
| | | X2947/X2949/X2954/X3750/X39/X3991/X3999/X4004/X452/X453/X4928/ |
| | | X510/X5146/X5149/X6345/X799/X84/X856 |
| ASN_seq_aaDown_R | −1.28 | X1/X1843/X19/X2703/X2708/X3745/X3762/X4920 |
| ASN_seq_aaDown_T | −1.28 | X1362/X1363/X1364/X2060/X2064/X2065/X2068/X265/X29/X2948/X2959/X2961/ |
| | | X4003/X4007/X512/X5148/X857/X859/X90 |
| ASN_seq_aaDown_V | −1.28 | X187/X192/X21/X390/X392/X708/X73 |
| ASN_seq_aaDown_W | −1.28 | X1184/X1852 |
| ASN_seq_aaDown_Y | −1.28 | X19/X3 |
| ASN_seq_aaUp_A | −1.28 | X10462/X10463/X10465/X10469/X10470/X10471/X10472/X10473/X10474/X10483/ |
| | | X10484/X10485/X10494/X10495/X10496/X10499/X105/X10501/X10503/ |
| | | X10505/X10508/X10510/X11108/X11112/X11117/X11118/X11119/X11134/ |
| | | X11137/X11139/X11585/X11602/X1167/X1168/X1170/X1175/X1177/X1180/ |
| | | X1183/X1185/X1191/X1193/X1283/X1284/X1439/X1441/X1839/X185/X1851/ |
| | | X1853/X1856/X1858/X1861/X190/X193/X194/X195/X196/X1980/X2120/ |
| | | X2161/X2163/X2164/X2165/X23/X241/X2728/X2732/X2733/X2735/X2996/X2997/ |
| | | X3002/X3004/X3064/X3066/X3068/X3069/X3071/X3074/X3075/X3782/ |
| | | X3783/X387/X389/X391/X395/X397/X399/X401/X402/X4025/X4026/X4032/ |
| | | X4033/X4037/X4038/X4039/X4040/X4042/X4116/X4117/X4122/X4125/X4126/ |
| | | X4128/X4129/X4131/X4133/X4134/X4136/X4137/X4138/X461/X464/X5161/ |
| | | X5164/X5165/X5169/X5170/X5171/X5172/X5173/X5174/X5178/X5179/X5180/ |
| | | X5181/X5262/X5265/X5267/X5268/X5269/X5270/X5272/X5274/X5275/ |
| | | X5277/X5279/X5280/X5281/X5284/X5285/X5286/X5287/X5288/X5290/X5291/ |
| | | X5293/X5297/X5298/X5299/X5300/X5301/X5302/X6353/X6355/X6356/X6357/ |
| | | X6358/X6359/X6363/X6364/X6365/X6366/X6367/X6368/X6369/X6445/ |
| | | X6447/X6448/X6449/X6450/X6453/X6454/X6455/X6456/X6457/X6458/X6459/ |
| | | X6460/X6462/X6465/X6466/X6467/X6468/X6469/X6471/X6472/X6474/X6475/ |
| | | X6477/X6479/X6480/X6481/X6482/X6483/X6484/X6486/X6487/X6490/ |
| | | X6491/X6495/X6496/X6497/X6498/X6499/X6500/X6501/X6502/X699/X701/ |
| | | X702/X706/X709/X71/X711/X714/X715/X717/X719/X75/X7521/X7522/X7524/ |
| | | X7525/X7526/X7527/X7528/X7529/X7530/X7591/X7594/X7595/X7596/X7597/ |
| | | X7598/X7599/X76/X7600/X7602/X7603/X7604/X7605/X7606/X7607/X7608/ |
| | | X7609/X7610/X7611/X7614/X7615/X7616/X7618/X7619/X7621/X7623/ |
| | | X7625/X7626/X7627/X7628/X7629/X7631/X7632/X7635/X7636/X7637/X7638/ |
| | | X7640/X7641/X7643/X7644/X7647/X7649/X7651/X7652/X7654/X7655/X7656/ |
| | | X7657/X7658/X7659/X7660/X7661/X789/X792/X795/X83/X8560/X8633/ |
| | | X8634/X8635/X8636/X8676/X8678/X8680/X8681/X8682/X8683/X8684/X8685/ |
| | | X8686/X8687/X8688/X8689/X8692/X8693/X8694/X8695/X8696/X8697/X8698/ |
| | | X8699/X8700/X8701/X8707/X8708/X8709/X8710/X8711/X8712/X8713/ |
| | | X8715/X8717/X8718/X8720/X8721/X8724/X8726/X8728/X8729/X8730/X8731/ |
| | | X8733/X8734/X8737/X8740/X8742/X8743/X8744/X8745/X8746/X8747/X8748/ |
| | | X9639/X9640/X9641/X9643/X9644/X9645/X9646/X9647/X9648/X9649/ |
| | | X9650/X9651/X9652/X9658/X9659/X9660/X9661/X9662/X9663/X9672/X9673/ |
| | | X9674/X9675/X9676/X9677/X9678/X9680/X9681/X9684/X9687/X9689/X9690/ |
| | | X9691/X9694/X9696/X9698/X9700/X9701/X9702 |
| ASN_seq_aaUp_C | −1.28 | X10494/X1176/X1179/X1193/X1857/X1869/X187/X189/X192/X21/X2120/X2996/ |
| | | X2997/X3002/X3004/X388/X390/X392/X394/X4025/X4026/X4032/X4033/ |
| | | X4037/X4038/X4039/X4040/X4042/X5161/X5164/X5165/X5169/X5170/X5171/ |
| | | X5172/X5173/X5174/X5178/X5179/X5180/X5181/X5287/X5293/X6353/ |
| | | X6355/X6356/X6357/X6358/X6359/X6363/X6364/X6365/X6366/X6367/X6368/ |
| | | X6369/X6462/X6468/X6483/X6490/X6491/X69/X70/X700/X705/X708/X710/ |
| | | X719/X73/X7521/X7522/X7524/X7525/X7526/X7527/X7528/X7529/X7530/ |
| | | X7615/X7618/X7621/X7628/X7637/X7651/X7652/X8633/X8634/X8635/X8636/ |
| | | X8707/X8709/X8711/X8712/X8715/X8730/X8742/X9672/X9674/X9676/X9677/ |
| | | X9689 |
| ASN_seq_aaUp_D | −1.28 | X1168/X1170/X1175/X1177/X1180/X1182/X1183/X1185/X1839/X185/X1850/ |
| | | X1851/X1853/X1856/X1858/X1860/X1861/X190/X2727/X2728/X2732/X2733/ |
| | | X2735/X2738/X3779/X3782/X3783/X387/X389/X391/X395/X4956/X699/X701/ |
| | | X702/X706/X709/X71/X711 |
| ASN_seq_aaUp_E | −1.28 | X1206/X1207/X1883/X198/X25/X406/X407/X727/X728/X729/X78/X83 |
| ASN_seq_aaUp_F | −1.28 | X104/X110/X1328/X1329/X1330/X2083/X234/X235/X255/X39/X452/X453/X491/ |
| | | X492/X493/X799/X829/X830/X831/X832 |
| ASN_seq_aaUp_H | −1.28 | X188/X708 |
| ASN_seq_aaUp_I | −1.28 | X1176/X1179/X1181/X1206/X1207/X1857/X187/X1883/X189/X192/X198/X21/ |
| | | X25/X388/X390/X392/X394/X396/X406/X407/X70/X700/X705/X708/X710/ |
| | | X727/X728/X729/X73/X78 |
| ASN_seq_aaUp_K | −1.28 | X1379/X2078/X871 |
| ASN_seq_aaUp_L | −1.28 | X10462/X10463/X10465/X10469/X10470/X10471/X10472/X10473/X10474/X10483/ |
| | | X10484/X10485/X10494/X10495/X10496/X10499/X10501/X10503/X10505/ |
| | | X10508/X10510/X11108/X11112/X11117/X11118/X11119/X11134/X11137/ |
| | | X11139/X11585/X11602/X1439/X1441/X2120/X2161/X2163/X2164/X2165/ |
| | | X2996/X2997/X3002/X3004/X3064/X3066/X3068/X3069/X3071/X3074/X3075/ |
| | | X4025/X4026/X4032/X4033/X4037/X4038/X4039/X4040/X4042/X4116/ |
| | | X4117/X4122/X4125/X4126/X4128/X4129/X4131/X4133/X4134/X4136/X4137/ |
| | | X4138/X5161/X5164/X5165/X5169/X5170/X5171/X5172/X5173/X5174/X5178/ |
| | | X5179/X5180/X5181/X5262/X5265/X5267/X5268/X5269/X5270/X5272/ |
| | | X5274/X5275/X5277/X5279/X5280/X5281/X5284/X5285/X5286/X5287/X5288/ |
| | | X5290/X5291/X5293/X5297/X5298/X5299/X5300/X5301/X5302/X6353/X6355/ |
| | | X6356/X6357/X6358/X6359/X6363/X6364/X6365/X6366/X6367/X6368/ |
| | | X6369/X6445/X6447/X6448/X6449/X6450/X6453/X6454/X6455/X6456/X6457/ |
| | | X6458/X6459/X6460/X6462/X6465/X6466/X6467/X6468/X6469/X6471/X6472/ |
| | | X6474/X6475/X6477/X6479/X6480/X6481/X6482/X6483/X6484/X6486/ |
| | | X6487/X6490/X6491/X6495/X6496/X6497/X6498/X6499/X6500/X6501/X6502/ |
| | | X7521/X7522/X7524/X7525/X7526/X7527/X7528/X7529/X7530/X7591/X7594/ |
| | | X7595/X7596/X7597/X7598/X7599/X7600/X7602/X7603/X7604/X7605/ |
| | | X7606/X7607/X7608/X7609/X7610/X7611/X7614/X7615/X7616/X7618/X7619/ |
| | | X7621/X7623/X7625/X7626/X7627/X7628/X7629/X7631/X7632/X7635/X7636/ |
| | | X7637/X7638/X7640/X7641/X7643/X7644/X7647/X7649/X7651/X7652/ |
| | | X7654/X7655/X7656/X7657/X7658/X7659/X7660/X7661/X8633/X8634/X8635/ |
| | | X8636/X8676/X8678/X8680/X8681/X8682/X8683/X8684/X8685/X8686/X8687/ |
| | | X8688/X8689/X8692/X8693/X8694/X8695/X8696/X8697/X8698/X8699/ |
| | | X8700/X8701/X8707/X8708/X8709/X8710/X8711/X8712/X8713/X8715/X8717/ |
| | | X8718/X8720/X8721/X8724/X8726/X8728/X8729/X8730/X8731/X8733/X8734/ |
| | | X8737/X8740/X8742/X8743/X8744/X8745/X8746/X8747/X8748/X9639/ |
| | | X9640/X9641/X9643/X9644/X9645/X9646/X9647/X9648/X9649/X9650/X9651/ |
| | | X9652/X9658/X9659/X9660/X9661/X9662/X9663/X9672/X9673/X9674/X9675/ |
| | | X9676/X9677/X9678/X9680/X9681/X9684/X9687/X9689/X9690/X9691/ |
| | | X9694/X9696/X9698/X9700/X9701/X9702 |
| ASN_seq_aaUp_N | −1.28 | X1168/X1175/X1182/X1183/X1839/X1850/X1851/X1860/X1861/X2727/X2728/ |
| | | X2732/X2738/X3779/X3782/X3783/X387/X4956/X702/X709 |
| ASN_seq_aaUp_P | −1.28 | X1188/X1189/X1190/X1191/X1192/X1193/X1194/X1865/X1867/X1868/X1869/ |
| | | X1894/X193/X194/X195/X196/X23/X2741/X2743/X2763/X3802/X397/X398/ |
| | | X399/X400/X401/X402/X713/X714/X715/X716/X717/X718/X719/X75/X76 |
| ASN_seq_aaUp_R | −1.28 | X189/X388/X394/X70/X700 |
| ASN_seq_aaUp_S | −1.28 | X10469/X10472/X10473/X10483/X10494/X10495/X11117/X1167/X1168/X1170/ |
| | | X1175/X1177/X1180/X1183/X1185/X1322/X1356/X1359/X1439/X1835/X1839/ |
| | | X1848/X185/X1851/X1853/X1856/X1858/X1861/X190/X2016/X2017/X2022/ |
| | | X2055/X2057/X2063/X2120/X2161/X2163/X2704/X2723/X2725/X2728/ |
| | | X2733/X2900/X2901/X2914/X2944/X2946/X2951/X2953/X2957/X2996/X2997/ |
| | | X3002/X3004/X3064/X3066/X3074/X3773/X3774/X387/X389/X391/X395/ |
| | | X3954/X3955/X3959/X3992/X3993/X3995/X3996/X3998/X4002/X4006/X4025/ |
| | | X4026/X4032/X4033/X4037/X4038/X4039/X4040/X4042/X4116/X4117/X4125/ |
| | | X4129/X4133/X4134/X4950/X4951/X5107/X5108/X5141/X5142/X5144/ |
| | | X5145/X5147/X5151/X5154/X5161/X5164/X5165/X5169/X5170/X5171/X5172/ |
| | | X5173/X5174/X5178/X5179/X5180/X5181/X5262/X5265/X5267/X5268/X5270/ |
| | | X5275/X5279/X5280/X5284/X5285/X5286/X5287/X5290/X5293/X6193/ |
| | | X6344/X6346/X6348/X6349/X6352/X6353/X6355/X6356/X6357/X6358/X6359/ |
| | | X6363/X6364/X6365/X6366/X6367/X6368/X6369/X6445/X6447/X6448/X6450/ |
| | | X6453/X6456/X6457/X6459/X6462/X6465/X6466/X6467/X6468/X6471/ |
| | | X6475/X6479/X6480/X6481/X6482/X6483/X6490/X6491/X699/X701/X702/X706/ |
| | | X709/X71/X711/X7517/X7519/X7520/X7521/X7522/X7524/X7525/X7526/ |
| | | X7527/X7528/X7529/X7530/X7594/X7597/X7598/X7600/X7602/X7603/X7605/ |
| | | X7606/X7609/X7610/X7615/X7616/X7618/X7621/X7623/X7625/X7626/ |
| | | X7627/X7628/X7635/X7636/X7637/X7640/X7643/X7651/X7652/X855/X8633/ |
| | | X8634/X8635/X8636/X8680/X8681/X8683/X8684/X8687/X8688/X8692/X8695/ |
| | | X8696/X8698/X8699/X8707/X8708/X8709/X8710/X8711/X8712/X8715/X8716/ |
| | | X8717/X8720/X8728/X8729/X8730/X8742/X9643/X9646/X9647/X9649/ |
| | | X9650/X9658/X9661/X9662/X9672/X9673/X9674/X9675/X9676/X9677/X9689/ |
| | | X9690 |
| ASN_seq_aaUp_V | −1.28 | X110/X1329/X1330/X255/X491/X492/X493/X830/X831/X832 |
| ASN_seq_hydrophobicity_kd | −1.28 | X10462/X10463/X10465/X10466/X10469/X10470/X10471/X10472/X10473/X10474/ |
| | | X10475/X10477/X10478/X10479/X10480/X10481/X10483/X10484/X10485/ |
| | | X10486/X10487/X10488/X10489/X10491/X10492/X10493/X10495/X10497/ |
| | | X10498/X10500/X10502/X10504/X10506/X10507/X10509/X10512/X10514/ |
| | | X10517/X10519/X10524/X11108/X11109/X11112/X11114/X11117/X11118/ |
| | | X11119/X11120/X11121/X11122/X11123/X11125/X11126/X11127/X11128/ |
| | | X11129/X11130/X11131/X11132/X11135/X11136/X11138/X11140/X11142/X11144/ |
| | | X11147/X113/X11585/X11587/X11590/X11593/X11594/X11595/X11596/ |
| | | X11597/X11599/X11600/X11601/X11603/X11605/X11898/X11901/X11904/ |
| | | X11905/X11906/X12091/X1344/X1345/X1346/X1347/X1357/X1360/X1439/ |
| | | X1440/X18/X187/X188/X192/X2042/X2043/X2044/X2045/X2058/X2059/X2061/ |
| | | X21/X214/X2161/X2162/X2163/X2166/X254/X258/X2932/X2933/X2934/ |
| | | X2945/X2947/X2949/X2954/X3063/X3064/X3065/X3066/X3070/X3073/X3074/ |
| | | X390/X392/X3980/X3981/X3991/X3999/X4004/X4114/X4115/X4116/X4117/ |
| | | X4118/X4119/X4124/X4125/X4127/X4129/X4130/X4131/X4133/X4134/X489/ |
| | | X502/X510/X5146/X5149/X5262/X5263/X5264/X5265/X5266/X5267/X5268/ |
| | | X5269/X5270/X5273/X5275/X5276/X5277/X5279/X5280/X5283/X5284/X5285/ |
| | | X5286/X5287/X5288/X5290/X5292/X6345/X6445/X6446/X6447/X6448/ |
| | | X6449/X6450/X6451/X6452/X6453/X6454/X6455/X6456/X6457/X6458/X6459/ |
| | | X6463/X6465/X6466/X6467/X6468/X6469/X6471/X6473/X6475/X6476/X6477/ |
| | | X6479/X6480/X6481/X6482/X6483/X6484/X6485/X6488/X6489/X6519/ |
| | | X708/X73/X7591/X7592/X7593/X7594/X7595/X7596/X7597/X7598/X7599/X7600/ |
| | | X7601/X7602/X7603/X7604/X7605/X7606/X7607/X7608/X7609/X7610/ |
| | | X7611/X7612/X7616/X7617/X7619/X7623/X7625/X7626/X7627/X7628/X7629/ |
| | | X7630/X7633/X7634/X7635/X7636/X7637/X7638/X7640/X7642/X7643/X7645/ |
| | | X7646/X7648/X7650/X7666/X7669/X7677/X84/X843/X844/X856/X8676/ |
| | | X8678/X8679/X8680/X8681/X8682/X8683/X8684/X8685/X8686/X8687/X8688/ |
| | | X8689/X8690/X8692/X8693/X8694/X8695/X8696/X8697/X8698/X8699/X8700/ |
| | | X8701/X8702/X8703/X8704/X8705/X8708/X8710/X8711/X8713/X8717/ |
| | | X8719/X8720/X8722/X8723/X8725/X8727/X8728/X8729/X8730/X8731/X8732/ |
| | | X8735/X8736/X8738/X8739/X8741/X8749/X8751/X8755/X8760/X8766/X907/ |
| | | X9639/X9640/X9641/X9643/X9644/X9645/X9646/X9647/X9648/X9649/X9650/ |
| | | X9651/X9652/X9653/X9654/X9655/X9656/X9658/X9659/X9660/X9661/ |
| | | X9662/X9663/X9664/X9666/X9667/X9668/X9669/X9670/X9673/X9675/X9676/ |
| | | X9678/X9679/X9682/X9683/X9685/X9686/X9688/X9690/X9692/X9693/X9695/ |
| | | X9697/X9699/X9703/X9705/X9707/X9710/X9716/X9720 |
| ASN_seq_RSA_accpro20 | −1.28 | X117/X1195/X1198/X13/X1354/X1366/X157/X1870/X1873/X1875/X190/X2070/ |
| | | X2231/X263/X2745/X2747/X2761/X3131/X3133/X3153/X3747/X3766/X3790/ |
| | | X3801/X389/X395/X4174/X4196/X45/X4917/X4932/X4967/X508/X509/ |
| | | X5349/X6168/X6184/X701/X7088/X71/X720/X8213/X851/X852 |
| ASN_seq_RSA_accproe | −1.28 | X1357/X1360/X1362/X1363/X1364/X2058/X2059/X2060/X2061/X2064/X2065/ |
| | | X2068/X214/X265/X29/X2945/X2948/X2949/X2954/X2959/X2961/X3991/ |
| | | X4003/X4004/X4007/X510/X512/X5146/X5148/X84/X856/X857/X859/X90 |
| ASN_seq_SS_sspro8C | −1.28 | X10462/X10463/X10465/X10469/X10470/X10471/X10472/X10473/X10474/X10483/ |
| | | X10484/X10485/X10494/X10495/X10496/X10499/X10501/X10503/X10505/ |
| | | X10508/X10510/X11108/X11112/X11117/X11118/X11119/X11134/X11137/ |
| | | X11139/X11585/X11602/X1177/X1180/X1357/X1360/X1439/X1440/X1441/ |
| | | X1856/X1858/X2058/X2059/X2061/X2120/X214/X2161/X2162/X2163/X2164/ |
| | | X2165/X2166/X254/X2732/X2945/X2947/X2949/X2954/X2996/X2997/X3002/ |
| | | X3004/X3064/X3065/X3066/X3068/X3069/X3070/X3071/X3073/X3074/ |
| | | X3075/X3782/X387/X395/X3991/X3999/X4004/X4025/X4026/X4032/X4033/ |
| | | X4037/X4038/X4039/X4040/X4042/X4114/X4116/X4117/X4119/X4122/X4124/ |
| | | X4125/X4126/X4127/X4128/X4129/X4130/X4131/X4133/X4134/X4136/X4137/ |
| | | X4138/X489/X49/X510/X5146/X5149/X5161/X5164/X5165/X5169/X5170/ |
| | | X5171/X5172/X5173/X5174/X5178/X5179/X5180/X5181/X5262/X5264/X5265/ |
| | | X5267/X5268/X5269/X5270/X5272/X5273/X5274/X5275/X5276/X5277/ |
| | | X5279/X5280/X5281/X5283/X5284/X5285/X5286/X5287/X5288/X5290/X5291/ |
| | | X5293/X5297/X5298/X5299/X5300/X5301/X5302/X6345/X6353/X6355/X6356/ |
| | | X6357/X6358/X6359/X6363/X6364/X6365/X6366/X6367/X6368/X6369/ |
| | | X6445/X6447/X6448/X6449/X6450/X6452/X6453/X6454/X6455/X6456/X6457/ |
| | | X6458/X6459/X6460/X6462/X6463/X6465/X6466/X6467/X6468/X6469/X6471/ |
| | | X6472/X6474/X6475/X6476/X6477/X6479/X6480/X6481/X6482/X6483/ |
| | | X6484/X6486/X6487/X6490/X6491/X6495/X6496/X6497/X6498/X6499/X6500/ |
| | | X6501/X6502/X701/X706/X711/X7521/X7522/X7524/X7525/X7526/X7527/ |
| | | X7528/X7529/X7530/X7591/X7593/X7594/X7595/X7596/X7597/X7598/X7599/ |
| | | X7600/X7602/X7603/X7604/X7605/X7606/X7607/X7608/X7609/X7610/X7611/ |
| | | X7614/X7615/X7616/X7617/X7618/X7619/X7621/X7623/X7625/X7626/ |
| | | X7627/X7628/X7629/X7631/X7632/X7635/X7636/X7637/X7638/X7640/X7641/ |
| | | X7643/X7644/X7647/X7649/X7651/X7652/X7654/X7655/X7656/X7657/X7658/ |
| | | X7659/X7660/X7661/X799/X830/X84/X856/X8633/X8634/X8635/X8636/ |
| | | X8676/X8678/X8680/X8681/X8682/X8683/X8684/X8685/X8686/X8687/X8688/ |
| | | X8689/X8692/X8693/X8694/X8695/X8696/X8697/X8698/X8699/X8700/X8701/ |
| | | X8707/X8708/X8709/X8710/X8711/X8712/X8713/X8715/X8717/X8718/ |
| | | X8720/X8721/X8724/X8726/X8728/X8729/X8730/X8731/X8733/X8734/X8737/ |
| | | X8740/X8742/X8743/X8744/X8745/X8746/X8747/X8748/X907/X9639/X9640/ |
| | | X9641/X9643/X9644/X9645/X9646/X9647/X9648/X9649/X9650/X9651/X9652/ |
| | | X9658/X9659/X9660/X9661/X9662/X9663/X9672/X9673/X9674/X9675/ |
| | | X9676/X9677/X9678/X9680/X9681/X9684/X9687/X9689/X9690/X9691/X9694/ |
| | | X9696/X9698/X9700/X9701/X9702 |
| ASN_seq_SS_sspro8E | −1.28 | X1166/X1847/X1849/X2724/X3775 |
| ASN_seq_SS_sspro8H | −1.28 | X453/X4928 |
| ASN_seq_SS_sspro8S | −1.28 | X1176/X1179/X1181/X1206/X1207/X1857/X187/X1883/X189/X192/X198/X21/ |
| | | X25/X388/X390/X392/X394/X396/X406/X407/X70/X700/X705/X707/X708/ |
| | | X710/X727/X728/X729/X73/X78 |
| ASN_seq_SS_sspro8T | −1.28 | X1179/X1857/X189/X19/X3/X388/X394/X70/X700/X705 |
| ASN_seq_SS_ssproE | −1.28 | X1166/X1181/X1847/X1849/X2724/X3775 |
| ASN_seq_SS_ssproH | −1.28 | X4928/X799 |
| ASN_struct_aa_A | −1.28 | X10462/X10463/X10465/X10469/X10470/X10471/X10472/X10473/X10474/X10483/ |
| | | X10484/X10485/X10494/X10495/X10496/X10499/X10501/X10503/X10505/ |
| | | X10508/X10510/X11108/X11112/X11117/X11118/X11119/X11134/X11137/ |
| | | X11139/X11585/X11602/X1177/X1180/X1356/X1359/X1439/X1441/X190/ |
| | | X2055/X2057/X2063/X2120/X2161/X2163/X2164/X2165/X2944/X2946/X2951/ |
| | | X2953/X2957/X2996/X2997/X3002/X3004/X3064/X3066/X3068/X3069/ |
| | | X3071/X3074/X3075/X389/X395/X3992/X3993/X3995/X3996/X3998/X4002/ |
| | | X4006/X4025/X4026/X4032/X4033/X4037/X4038/X4039/X4040/X4042/X4116/ |
| | | X4117/X4122/X4125/X4126/X4128/X4129/X4131/X4133/X4134/X4136/X4137/ |
| | | X4138/X5141/X5142/X5144/X5145/X5147/X5151/X5154/X5161/X5164/ |
| | | X5165/X5169/X5170/X5171/X5172/X5173/X5174/X5178/X5179/X5180/X5181/ |
| | | X5262/X5265/X5267/X5268/X5269/X5270/X5272/X5274/X5275/X5277/X5279/ |
| | | X5280/X5281/X5284/X5285/X5286/X5287/X5288/X5290/X5291/X5293/ |
| | | X5297/X5298/X5299/X5300/X5301/X5302/X6344/X6346/X6348/X6349/X6352/ |
| | | X6353/X6355/X6356/X6357/X6358/X6359/X6363/X6364/X6365/X6366/X6367/ |
| | | X6368/X6369/X6445/X6447/X6448/X6449/X6450/X6453/X6454/X6455/ |
| | | X6456/X6457/X6458/X6459/X6460/X6462/X6465/X6466/X6467/X6468/X6469/ |
| | | X6471/X6472/X6474/X6475/X6477/X6479/X6480/X6481/X6482/X6483/X6484/ |
| | | X6486/X6487/X6490/X6491/X6495/X6496/X6497/X6498/X6499/X6500/ |
| | | X6501/X6502/X701/X706/X71/X711/X7517/X7519/X7520/X7521/X7522/X7524/ |
| | | X7525/X7526/X7527/X7528/X7529/X7530/X7591/X7594/X7595/X7596/X7597/ |
| | | X7598/X7599/X7600/X7602/X7603/X7604/X7605/X7606/X7607/X7608/ |
| | | X7609/X7610/X7611/X7614/X7615/X7616/X7618/X7619/X7621/X7623/X7625/ |
| | | X7626/X7627/X7628/X7629/X7631/X7632/X7635/X7636/X7637/X7638/X7640/ |
| | | X7641/X7643/X7644/X7647/X7649/X7651/X7652/X7654/X7655/X7656/ |
| | | X7657/X7658/X7659/X7660/X7661/X855/X8560/X8633/X8634/X8635/X8636/ |
| | | X8676/X8678/X8680/X8681/X8682/X8683/X8684/X8685/X8686/X8687/X8688/ |
| | | X8689/X8692/X8693/X8694/X8695/X8696/X8697/X8698/X8699/X8700/X8701/ |
| | | X8707/X8708/X8709/X8710/X8711/X8712/X8713/X8715/X8716/X8717/ |
| | | X8718/X8720/X8721/X8724/X8726/X8728/X8729/X8730/X8731/X8733/X8734/ |
| | | X8737/X8740/X8742/X8743/X8744/X8745/X8746/X8747/X8748/X9639/X9640/ |
| | | X9641/X9643/X9644/X9645/X9646/X9647/X9648/X9649/X9650/X9651/ |
| | | X9652/X9658/X9659/X9660/X9661/X9662/X9663/X9672/X9673/X9674/X9675/ |
| | | X9676/X9677/X9678/X9680/X9681/X9684/X9687/X9689/X9690/X9691/X9694/ |
| | | X9696/X9698/X9700/X9701/X9702 |
| ASN_struct_aa_C | −1.28 | X1179/X394/X705 |
| ASN_struct_aa_D | −1.28 | X16/X183/X386/X67/X704 |
| ASN_struct_aa_E | −1.28 | X10462/X10463/X10465/X10469/X10470/X10471/X10472/X10473/X10474/X10483/ |
| | | X10484/X10485/X10494/X10495/X11108/X11112/X11117/X11118/X11119/ |
| | | X11585/X1168/X1182/X1188/X1189/X1190/X1191/X1192/X1193/X1194/ |
| | | X1206/X1207/X1266/X1439/X1839/X1850/X1860/X1865/X1867/X1868/X1869/ |
| | | X1883/X1894/X193/X194/X195/X1952/X196/X198/X2120/X2161/X2163/ |
| | | X23/X25/X2727/X2732/X2738/X2741/X2743/X2763/X2827/X2996/X2997/X3002/ |
| | | X3004/X3064/X3066/X3074/X3779/X3782/X3802/X3865/X387/X397/X398/ |
| | | X399/X400/X401/X402/X4025/X4026/X4032/X4033/X4037/X4038/X4039/ |
| | | X4040/X4042/X406/X407/X4116/X4117/X4125/X4129/X4131/X4133/X4134/ |
| | | X4956/X5161/X5164/X5165/X5169/X5170/X5171/X5172/X5173/X5174/X5178/ |
| | | X5179/X5180/X5181/X5262/X5265/X5267/X5268/X5269/X5270/X5275/X5277/ |
| | | X5279/X5280/X5284/X5285/X5286/X5287/X5288/X5290/X5293/X6/X6353/ |
| | | X6355/X6356/X6357/X6358/X6359/X6363/X6364/X6365/X6366/X6367/X6368/ |
| | | X6369/X6445/X6447/X6448/X6449/X6450/X6453/X6454/X6455/X6456/ |
| | | X6457/X6458/X6459/X6462/X6465/X6466/X6467/X6468/X6469/X6471/X6475/ |
| | | X6477/X6479/X6480/X6481/X6482/X6483/X6484/X6490/X6491/X702/X713/ |
| | | X714/X715/X716/X717/X718/X719/X727/X728/X729/X75/X7521/X7522/X7524/ |
| | | X7525/X7526/X7527/X7528/X7529/X7530/X7591/X7594/X7595/X7596/ |
| | | X7597/X7598/X7599/X76/X7600/X7602/X7603/X7604/X7605/X7606/X7607/ |
| | | X7608/X7609/X7610/X7611/X7615/X7616/X7618/X7619/X7621/X7623/X7625/ |
| | | X7626/X7627/X7628/X7629/X7635/X7636/X7637/X7638/X7640/X7643/X7651/ |
| | | X7652/X78/X83/X8633/X8634/X8635/X8636/X8676/X8678/X8680/X8681/ |
| | | X8682/X8683/X8684/X8685/X8686/X8687/X8688/X8689/X8692/X8693/X8694/ |
| | | X8695/X8696/X8697/X8698/X8699/X8700/X8701/X8707/X8708/X8709/ |
| | | X8710/X8711/X8712/X8713/X8715/X8717/X8720/X8728/X8729/X8730/X8731/ |
| | | X8742/X9639/X9640/X9641/X9643/X9644/X9645/X9646/X9647/X9648/X9649/ |
| | | X9650/X9651/X9652/X9658/X9659/X9660/X9661/X9662/X9663/X9672/ |
| | | X9673/X9674/X9675/X9676/X9677/X9678/X9689/X9690 |
| ASN_struct_aa_H | −1.28 | X1206/X1207/X1883/X198/X25/X406/X407/X727/X728/X729/X78 |
| ASN_struct_aa_I | −1.28 | X110/X1199/X1202/X1328/X1329/X1330/X1876/X197/X2083/X24/X255/X3865/ |
| | | X403/X4130/X491/X492/X493/X5276/X6476/X722/X725/X7617/X77/X829/ |
| | | X830/X831/X832 |
| ASN_struct_aa_K | −1.28 | X10469/X10472/X10473/X10483/X10494/X10495/X110/X11117/X1328/X1329/ |
| | | X1330/X1439/X2083/X2120/X2161/X2163/X255/X2996/X2997/X3002/X3004/ |
| | | X3064/X3066/X3074/X4025/X4026/X4032/X4033/X4037/X4038/X4039/X4040/ |
| | | X4042/X4116/X4117/X4125/X4129/X4133/X4134/X491/X492/X493/X5161/ |
| | | X5164/X5165/X5169/X5170/X5171/X5172/X5173/X5174/X5178/X5179/ |
| | | X5180/X5181/X5262/X5265/X5267/X5268/X5270/X5275/X5279/X5280/X5284/ |
| | | X5285/X5286/X5287/X5290/X5293/X6353/X6355/X6356/X6357/X6358/X6359/ |
| | | X6363/X6364/X6365/X6366/X6367/X6368/X6369/X6445/X6447/X6448/ |
| | | X6450/X6453/X6456/X6457/X6459/X6462/X6465/X6466/X6467/X6468/X6471/ |
| | | X6475/X6479/X6480/X6481/X6482/X6483/X6490/X6491/X7521/X7522/X7524/ |
| | | X7525/X7526/X7527/X7528/X7529/X7530/X7594/X7597/X7598/X7600/ |
| | | X7602/X7603/X7605/X7606/X7609/X7610/X7615/X7616/X7618/X7621/X7623/ |
| | | X7625/X7626/X7627/X7628/X7635/X7636/X7637/X7640/X7643/X7651/X7652/ |
| | | X829/X830/X831/X832/X8633/X8634/X8635/X8636/X8680/X8681/X8683/ |
| | | X8684/X8687/X8688/X8692/X8695/X8696/X8698/X8699/X8707/X8708/X8709/ |
| | | X8710/X8711/X8712/X8715/X8717/X8720/X8728/X8729/X8730/X8742/ |
| | | X9643/X9646/X9647/X9649/X9650/X9658/X9661/X9662/X9672/X9673/X9674/ |
| | | X9675/X9676/X9677/X9689/X9690 |
| ASN_struct_aa_P | −1.28 | X1176/X1179/X1857/X187/X189/X19/X192/X21/X3/X388/X390/X392/X394/ |
| | | X396/X70/X700/X705/X707/X708/X710/X73 |
| ASN_struct_aa_R | −1.28 | X1176/X1179/X1189/X1329/X1857/X187/X189/X19/X192/X21/X3/X3775/X388/ |
| | | X390/X392/X394/X400/X70/X700/X705/X710/X718/X73 |
| ASN_struct_aa_S | −1.28 | X1267/X1953/X2828/X3865 |
| ASN_struct_aa_T | −1.28 | X1277/X1281/X1287/X1961/X1974/X1978/X1984/X239/X2855/X2861/X2868/ |
| | | X3907/X3922/X462/X787/X793 |
| ASN_struct_aa_V | −1.28 | X16/X183/X3775/X386/X67/X704 |
| ASN_struct_ASA_dssp | −1.28 | X1440/X2140/X2162/X2166/X254/X2947/X3065/X3070/X3073/X3999/X4114/ |
| | | X4119/X4124/X4127/X489/X5149/X5264/X5273/X5283/X6345/X6452/X6463/ |
| | | X7593/X907 |
| ASN_struct_ASA_BACKBONE_{—} | −1.28 | X10462/X10463/X10465/X10466/X10469/X10470/X10471/X10472/X10473/X10474/ |
| frecsasa_het | | X10475/X10477/X10478/X10479/X10480/X10481/X10483/X10484/X10485/ |
| | | X10486/X10487/X10488/X10489/X10491/X10492/X10493/X10495/X10497/ |
| | | X10498/X10500/X10502/X10504/X10506/X10507/X10509/X10512/X10514/ |
| | | X10517/X10519/X10524/X11108/X11109/X11112/X11114/X11117/X11118/ |
| | | X11119/X11120/X11121/X11122/X11123/X11125/X11126/X11127/X11128/ |
| | | X11129/X11130/X11131/X11132/X11135/X11136/X11138/X11140/X11142/X11144/ |
| | | X11147/X11585/X11587/X11590/X11593/X11594/X11595/X11596/X11597/ |
| | | X11599/X11600/X11601/X11603/X11605/X1188/X1189/X11898/X1190/ |
| | | X11901/X11904/X11905/X11906/X1191/X1192/X1193/X1194/X12091/X1439/ |
| | | X1865/X1866/X1867/X1868/X1869/X1894/X193/X194/X195/X196/X2161/X2163/ |
| | | X23/X2741/X2742/X2743/X2763/X2947/X3063/X3064/X3066/X3074/X3207/ |
| | | X3750/X3802/X3935/X397/X398/X399/X3999/X400/X401/X402/X4115/ |
| | | X4116/X4117/X4118/X4125/X4129/X4131/X4133/X4134/X5098/X5149/X5262/ |
| | | X5263/X5265/X5266/X5267/X5268/X5269/X5270/X5275/X5277/X5279/X5280/ |
| | | X5284/X5285/X5286/X5287/X5288/X5290/X5292/X6345/X6445/X6446/ |
| | | X6447/X6448/X6449/X6450/X6451/X6453/X6454/X6455/X6456/X6457/X6458/ |
| | | X6459/X6465/X6466/X6467/X6468/X6469/X6471/X6473/X6475/X6477/X6479/ |
| | | X6480/X6481/X6482/X6483/X6484/X6485/X6488/X6489/X6519/X713/X714/ |
| | | X715/X716/X717/X718/X719/X75/X7591/X7592/X7594/X7595/X7596/X7597/ |
| | | X7598/X7599/X76/X7600/X7601/X7602/X7603/X7604/X7605/X7606/X7607/ |
| | | X7608/X7609/X7610/X7611/X7612/X7616/X7619/X7623/X7625/X7626/ |
| | | X7627/X7628/X7629/X7630/X7633/X7634/X7635/X7636/X7637/X7638/X7640/ |
| | | X7642/X7643/X7645/X7646/X7648/X7650/X7666/X7669/X7677/X8676/X8678/ |
| | | X8679/X8680/X8681/X8682/X8683/X8684/X8685/X8686/X8687/X8688/ |
| | | X8689/X8690/X8692/X8693/X8694/X8695/X8696/X8697/X8698/X8699/X8700/ |
| | | X8701/X8702/X8703/X8704/X8705/X8708/X8710/X8711/X8713/X8717/X8719/ |
| | | X8720/X8722/X8723/X8725/X8727/X8728/X8729/X8730/X8731/X8732/ |
| | | X8735/X8736/X8738/X8739/X8741/X8749/X8751/X8755/X8760/X8766/X9639/ |
| | | X9640/X9641/X9643/X9644/X9645/X9646/X9647/X9648/X9649/X9650/X9651/ |
| | | X9652/X9653/X9654/X9655/X9656/X9658/X9659/X9660/X9661/X9662/ |
| | | X9663/X9664/X9666/X9667/X9668/X9669/X9670/X9673/X9675/X9676/X9678/ |
| | | X9679/X9682/X9683/X9685/X9686/X9688/X9690/X9692/X9693/X9695/X9697/ |
| | | X9699/X9703/X9705/X9707/X9710/X9716/X9720 |
| ASN_struct_ASA_{—} | −1.28 | X1357/X1360/X1440/X2058/X2059/X2061/X214/X2162/X2166/X254/X2945/ |
| NONPOLAR_freesasa_het | | X2947/X2949/X2954/X3065/X3070/X3073/X3207/X3991/X3999/X4004/X4114/ |
| | | X4119/X4124/X4127/X4130/X489/X510/X5146/X5149/X5264/X5273/X5276/ |
| | | X5283/X6345/X6452/X6463/X6476/X7593/X7617/X84/X856/X907 |
| ASN_struct_ASA_{—} | −1.28 | X1357/X1360/X1440/X2058/X2059/X2061/X214/X2140/X2162/X2166/X254/ |
| POLAR_freesasa_het | | X2945/X2947/X2949/X2954/X3065/X3070/X3073/X3991/X3999/X4004/X4114/ |
| | | X4119/X4124/X4127/X4130/X489/X510/X5146/X5149/X5264/X5273/X5276/ |
| | | X5283/X6345/X6452/X6463/X6476/X7593/X7617/X84/X856/X907 |
| ASN_struct_ASA_{—} | −1.28 | X1344/X1345/X1346/X1347/X1357/X1360/X1440/X2042/X2043/X2044/X2045/ |
| RESIDUE_freesasa_het | | X2058/X2059/X2061/X214/X2162/X2166/X254/X258/X2932/X2933/X2934/ |
| | | X2945/X2947/X2949/X2954/X3065/X3070/X3073/X3980/X3981/X3991/X3999/ |
| | | X4004/X4114/X4119/X4124/X4127/X4130/X489/X502/X510/X5146/X5149/ |
| | | X5264/X5273/X5276/X5283/X6345/X6452/X6463/X6476/X7593/X7617/X84/ |
| | | X843/X844/X856/X907 |
| ASN_struct_CA_DEPTH_msms | −1.28 | X1277/X1281/X1287/X1289/X170/X191/X1970/X1974/X1976/X1978/X1984/ |
| | | X239/X2843/X2851/X2861/X2868/X2870/X3885/X3892/X3922/X462/X5054/ |
| | | X74/X787/X793/X83 |
| ASN_struct_PHI_dssp | −1.28 | X1189/X1194/X1357/X1360/X1866/X1867/X2058/X2059/X2061/X214/X2742/ |
| | | X2743/X2945/X2947/X2949/X2954/X3935/X3991/X3999/X400/X4004/X5098/ |
| | | X510/X5146/X5149/X6345/X718/X84/X856 |
| ASN_struct_PSI_dssp | −1.28 | X170 |
| ASN_struct_RES_DEPTH_msms | −1.28 | X1189/X1194/X1866/X1867/X2742/X2743/X3935/X400/X5098/X718/X83 |
| ASN_struct_RSA_dssp | −1.28 | X1440/X2140/X2162/X2166/X254/X2947/X3065/X3070/X3073/X3999/X4114/ |
| | | X4119/X4124/X4127/X489/X5149/X5264/X5273/X5283/X6345/X6452/X6463/ |
| | | X7593/X907 |
| ASN_struct_RSA_{—} | −1.28 | X4920 |
| ALL_freesasa_het | | |
| ASN_struct_RSA_{—} | −1.28 | X10462/X10463/X10465/X10466/X10469/X10470/X10471/X10472/X10473/X10474/ |
| BACKBONE_freesasa_het | | X10475/X10477/X10478/X10479/X10480/X10481/X10483/X10484/X10485/ |
| | | X10486/X10487/X10488/X10489/X10491/X10492/X10493/X10495/X10497/ |
| | | X10498/X10500/X10502/X10504/X10506/X10507/X10509/X10512/X10514/ |
| | | X10517/X10519/X10524/X11108/X11109/X11112/X11114/X11117/X11118/ |
| | | X11119/X11120/X11121/X11122/X11123/X11125/X11126/X11127/X11128/ |
| | | X11129/X11130/X11131/X11132/X11135/X11136/X11138/X11140/X11142/X11144/ |
| | | X11147/X11585/X11587/X11590/X11593/X11594/X11595/X11596/X11597/ |
| | | X11599/X11600/X11601/X11603/X11605/X1188/X1189/X11898/X1190/ |
| | | X11901/X11904/X11905/X11906/X1191/X1192/X1193/X1194/X12091/X1330/ |
| | | X1356/X1359/X1439/X1865/X1866/X1867/X1868/X1869/X1894/X193/X194/ |
| | | X195/X196/X2055/X2057/X2063/X2161/X2163/X23/X2741/X2742/X2743/X2763/ |
| | | X2944/X2946/X2947/X2951/X2953/X2957/X3063/X3064/X3066/X3074/ |
| | | X3207/X3750/X3802/X3935/X397/X398/X399/X3992/X3993/X3995/X3996/X3998/ |
| | | X3999/X400/X4002/X4006/X401/X402/X4115/X4116/X4117/X4118/X4125/ |
| | | X4129/X4131/X4133/X4134/X49/X493/X5098/X5141/X5142/X5144/X5145/ |
| | | X5147/X5149/X5151/X5154/X5262/X5263/X5265/X5266/X5267/X5268/X5269/ |
| | | X5270/X5275/X5277/X5279/X5280/X5284/X5285/X5286/X5287/X5288/ |
| | | X5290/X5292/X6344/X6345/X6346/X6348/X6349/X6352/X6445/X6446/X6447/ |
| | | X6448/X6449/X6450/X6451/X6453/X6454/X6455/X6456/X6457/X6458/X6459/ |
| | | X6465/X6466/X6467/X6468/X6469/X6471/X6473/X6475/X6477/X6479/ |
| | | X6480/X6481/X6482/X6483/X6484/X6485/X6488/X6489/X6519/X713/X714/ |
| | | X715/X716/X717/X718/X719/X75/X7517/X7519/X7520/X7591/X7592/X7594/ |
| | | X7595/X7596/X7597/X7598/X7599/X76/X7600/X7601/X7602/X7603/X7604/ |
| | | X7605/X7606/X7607/X7608/X7609/X7610/X7611/X7612/X7616/X7619/X7623/ |
| | | X7625/X7626/X7627/X7628/X7629/X7630/X7633/X7634/X7635/X7636/X7637/ |
| | | X7638/X7640/X7642/X7643/X7645/X7646/X7648/X7650/X7666/X7669/ |
| | | X7677/X832/X855/X8676/X8678/X8679/X8680/X8681/X8682/X8683/X8684/ |
| | | X8685/X8686/X8687/X8688/X8689/X8690/X8692/X8693/X8694/X8695/X8696/ |
| | | X8697/X8698/X8699/X8700/X8701/X8702/X8703/X8704/X8705/X8708/X8710/ |
| | | X8711/X8713/X8716/X8717/X8719/X8720/X8722/X8723/X8725/X8727/ |
| | | X8728/X8729/X8730/X8731/X8732/X8735/X8736/X8738/X8739/X8741/X8749/ |
| | | X8751/X8755/X8760/X8766/X9639/X9640/X9641/X9643/X9644/X9645/X9646/ |
| | | X9647/X9648/X9649/X9650/X9651/X9652/X9653/X9654/X9655/X9656/ |
| | | X9658/X9659/X9660/X9661/X9662/X9663/X9664/X9666/X9667/X9668/X9669/ |
| | | X9670/X9673/X9675/X9676/X9678/X9679/X9682/X9683/X9685/X9686/X9688/ |
| | | X9690/X9692/X9693/X9695/X9697/X9699/X9703/X9705/X9707/X9710/ |
| | | X9716/X9720 |
| ASN_struct_RSA_{—} | −1.28 | X1357/X1360/X1440/X2058/X2059/X2061/X214/X2162/X2166/X254/X2945/ |
| NONPOLAR_freesasa_het | | X2947/X2949/X2954/X3065/X3070/X3073/X3207/X3991/X3999/X4004/X4114/ |
| | | X4119/X4124/X4127/X4130/X489/X510/X5146/X5149/X5264/X5273/X5276/ |
| | | X5283/X6345/X6452/X6463/X6476/X7593/X7617/X84/X856/X907 |
| ASN_struct_RSA_POLAR_{—} | −1.28 | X1357/X1360/X1440/X2058/X2059/X2061/X214/X2140/X2162/X2166/X254/ |
| freesasa_het | | X2945/X2947/X2949/X2954/X3065/X3070/X3073/X3991/X3999/X4004/X4114/ |
| | | X4119/X4124/X4127/X4130/X489/X510/X5146/X5149/X5264/X5273/X5276/ |
| | | X5283/X6345/X6452/X6463/X6476/X7593/X7617/X84/X856/X907 |
| ASN_struct_RSA_{—} | −1.28 | X1344/X1345/X1346/X1347/X1357/X1360/X1440/X2042/X2043/X2044/X2045/ |
| RESIDUE_freesasa_het | | X2058/X2059/X2061/X214/X2162/X2166/X254/X258/X2932/X2933/X2934/ |
| | | X2945/X2947/X2949/X2954/X3065/X3070/X3073/X3980/X3981/X3991/X3999/ |
| | | X4004/X4114/X4119/X4124/X4127/X4130/X489/X502/X510/X5146/X5149/ |
| | | X5264/X5273/X5276/X5283/X6345/X6452/X6463/X6476/X7593/X7617/X84/ |
| | | X843/X844/X856/X907 |
| ASN_struct_SS_dsspH | −1.28 | X1832/X2705/X2738/X3750/X3779/X453/X4928/X4956 |
| ASN_struct_SS_dsspS | −1.28 | X192/X25/X6 |
| ASN_struct_SS_dsspT | −1.28 | X1179/X189/X388/X394/X70/X700/X705 |
| SER.THR_seq_aaAll_I | −1.28 | X17/X96 |
| SER.THR_seq_aaUp_I | −1.28 | X17/X96 |
| SER.THR_seq_aaUp_S | −1.28 | X17 |
| SER.THR_seq_hydrophobicity_kd | −1.28 | X214/X84/X85/X86/X87/X88 |
| SER.THR_seq_RSA_accproe | −1.28 | X1 |
| ASN_seq_aaAll_A | −2.33 | X4929 |
| ASN_seq_aaAll_F | −2.33 | X6197 |
| ASN_seq_aaAll_I | −2.33 | X188 |
| ASN_seq_aaAll_R | −2.33 | X3763/X4958 |
| ASN_seq_aaAll_T | −2.33 | X1317/X1320/X1321/X2012/X2021/X2023/X253/X2910/X2913/X3958/X487/ |
| | | X823/X824 |
| ASN_seq_aaAll_V | −2.33 | X6174 |
| ASN_seq_aaUp_E | −2.33 | X6159/X7379/X8543 |
| ASN_seq_aaUp_L | −2.33 | X3749/X4927 |
| ASN_seq_SS_sspro8S | −2.33 | X8545/X8546 |
| ASN_struct_aa_F | −2.33 | X3743/X4926/X6173 |
| ASN_struct_aa_I | −2.33 | X188 |
| ASN_struct_aa_Q | −2.33 | X3781/X6197 |
| ASN_struct_aa_V | −2.33 | X1266/X1952/X2827 |
| ASN_struct_ASA_{—} | −2.33 | X1330/X493/X832 |
| BACKBONE_freesasa_het | | |
| ASN_struct_ASA_{—} | −2.33 | X10462/X10463/X10464/X10465/X10466/X10467/X10468/X10469/X10470/X10471/ |
| POLAR_freesasa_het | | X10472/X10473/X10474/X10475/X10476/X10477/X10478/X10479/X10480/ |
| | | X10481/X10482/X10483/X10484/X10485/X10486/X10487/X10488/X10489/ |
| | | X10490/X10491/X10492/X10493/X10494/X10495/X10497/X10498/X10500/ |
| | | X10502/X10504/X10506/X10507/X10509/X10512/X10513/X10514/X10517/ |
| | | X10519/X10523/X10524/X10527/X10529/X10538/X10549/X10558/X10568/ |
| | | X11108/X11109/X11110/X11111/X11112/X11113/X11114/X11115/X11116/X11117/ |
| | | X11118/X11119/X11120/X11121/X11122/X11123/X11124/X11125/X11126/ |
| | | X11127/X11128/X11129/X11130/X11131/X11132/X11133/X11135/X11136/ |
| | | X11138/X11140/X11142/X11144/X11147/X11150/X11163/X11174/X11180/ |
| | | X11584/X11585/X11586/X11587/X11588/X11589/X11590/X11591/X11592/ |
| | | X11593/X11594/X11595/X11596/X11597/X11598/X11599/X11600/X11601/ |
| | | X11603/X11605/X11618/X11627/X11897/X11898/X11899/X11900/X11901/X11902/ |
| | | X11903/X11904/X11905/X11906/X11916/X12090/X12091/X12092/X12093/ |
| | | X12200/X1439/X2120/X2161/X2163/X2243/X2996/X2997/X3002/X3004/ |
| | | X3063/X3064/X3066/X3074/X3148/X3184/X4025/X4026/X4032/X4033/X4037/ |
| | | X4038/X4039/X4040/X4042/X4115/X4116/X4117/X4118/X4125/X4129/ |
| | | X4131/X4133/X4134/X4188/X4190/X4232/X5161/X5164/X5165/X5169/X5170/ |
| | | X5171/X5172/X5173/X5174/X5178/X5179/X5180/X5181/X5261/X5262/X5263/ |
| | | X5265/X5266/X5267/X5268/X5269/X5270/X5275/X5277/X5279/X5280/ |
| | | X5284/X5285/X5286/X5287/X5288/X5290/X5292/X5293/X5334/X5336/X5338/ |
| | | X5345/X5387/X5388/X6353/X6355/X6356/X6357/X6358/X6359/X6363/X6364/ |
| | | X6365/X6366/X6367/X6368/X6369/X6444/X6445/X6446/X6447/X6448/ |
| | | X6449/X6450/X6451/X6453/X6454/X6455/X6456/X6457/X6458/X6459/X6462/ |
| | | X6465/X6466/X6467/X6468/X6469/X6471/X6473/X6475/X6477/X6479/X6480/ |
| | | X6481/X6482/X6483/X6484/X6485/X6488/X6489/X6490/X6491/X6515/ |
| | | X6517/X6518/X6519/X6523/X6524/X6529/X6534/X6567/X6568/X6569/X7521/ |
| | | X7522/X7524/X7525/X7526/X7527/X7528/X7529/X7530/X7590/X7591/X7592/ |
| | | X7594/X7595/X7596/X7597/X7598/X7599/X7600/X7601/X7602/X7603/ |
| | | X7604/X7605/X7606/X7607/X7608/X7609/X7610/X7611/X7612/X7613/X7615/ |
| | | X7616/X7618/X7619/X7621/X7623/X7625/X7626/X7627/X7628/X7629/X7630/ |
| | | X7633/X7634/X7635/X7636/X7637/X7638/X7640/X7642/X7643/X7645/ |
| | | X7646/X7648/X7650/X7651/X7652/X7666/X7667/X7668/X7669/X7673/X7674/ |
| | | X7676/X7677/X7681/X7686/X7690/X7692/X7720/X7721/X7722/X7723/X8633/ |
| | | X8634/X8635/X8636/X8676/X8677/X8678/X8679/X8680/X8681/X8682/ |
| | | X8683/X8684/X8685/X8686/X8687/X8688/X8689/X8690/X8691/X8692/X8693/ |
| | | X8694/X8695/X8696/X8697/X8698/X8699/X8700/X8701/X8702/X8703/X8704/ |
| | | X8705/X8706/X8707/X8708/X8709/X8710/X8711/X8712/X8713/X8715/ |
| | | X8717/X8719/X8720/X8722/X8723/X8725/X8727/X8728/X8729/X8730/X8731/ |
| | | X8732/X8735/X8736/X8738/X8739/X8741/X8742/X8749/X8750/X8751/X8754/ |
| | | X8755/X8759/X8760/X8764/X8765/X8766/X8769/X8773/X8775/X8778/ |
| | | X8800/X8801/X8802/X8811/X9638/X9639/X9640/X9641/X9642/X9643/X9644/ |
| | | X9645/X9646/X9647/X9648/X9649/X9650/X9651/X9652/X9653/X9654/X9655/ |
| | | X9656/X9657/X9658/X9659/X9660/X9661/X9662/X9663/X9664/X9665/ |
| | | X9666/X9667/X9668/X9669/X9670/X9671/X9672/X9673/X9674/X9675/X9676/ |
| | | X9677/X9678/X9679/X9682/X9683/X9685/X9686/X9688/X9689/X9690/X9692/ |
| | | X9693/X9695/X9697/X9699/X9703/X9705/X9706/X9707/X9710/X9714/ |
| | | X9715/X9716/X9719/X9720/X9724/X9727/X9729/X9744/X9745/X9750/X9765 |
| ASN_struct_ASA_{—} | −2.33 | X10466/X10475/X10477/X10478/X10479/X10480/X10481/X10486/X10487/X10488/ |
| RESIDUE_freesasa_het | | X10489/X10491/X10492/X10493/X10497/X10498/X10500/X10502/X10504/ |
| | | X10506/X10507/X10509/X10512/X10514/X10517/X10519/X10524/X11109/ |
| | | X11114/X11120/X11121/X11122/X11123/X11125/X11126/X11127/X11128/ |
| | | X11129/X11130/X11131/X11132/X11135/X11136/X11138/X11140/X11142/ |
| | | X11144/X11147/X11587/X11590/X11593/X11594/X11595/X11596/X11597/ |
| | | X11599/X11600/X11601/X11603/X11605/X11898/X11901/X11904/X11905/X11906/ |
| | | X12091/X3063/X4115/X4118/X5263/X5266/X5292/X6446/X6451/X6473/ |
| | | X6485/X6488/X6489/X6519/X7592/X7601/X7612/X7630/X7633/X7634/X7642/ |
| | | X7645/X7646/X7648/X7650/X7666/X7669/X7677/X8679/X8690/X8702/ |
| | | X8703/X8704/X8705/X8719/X8722/X8723/X8725/X8727/X8732/X8735/X8736/ |
| | | X8738/X8739/X8741/X8749/X8751/X8755/X8760/X8766/X9653/X9654/X9655/ |
| | | X9656/X9664/X9666/X9667/X9668/X9669/X9670/X9679/X9682/X9683/ |
| | | X9685/X9686/X9688/X9692/X9693/X9695/X9697/X9699/X9703/X9705/X9707/ |
| | | X9710/X9716/X9720 |
| ASN_struct_PSI_dssp | −2.33 | X356/X8556 |
| ASN_struct_RES_{—} | −2.33 | X170 |
| DEPTH_msms | | |
| ASN_struct_RSA_{—} | −2.33 | X10462/X10463/X10464/X10465/X10466/X10467/X10468/X10469/X10470/X10471/ |
| POLAR_freesasa_het | | X10472/X10473/X10474/X10475/X10476/X10477/X10478/X10479/X10480/ |
| | | X10481/X10482/X10483/X10484/X10485/X10486/X10487/X10488/X10489/ |
| | | X10490/X10491/X10492/X10493/X10495/X10497/X10498/X10500/X10502/ |
| | | X10504/X10506/X10507/X10509/X10512/X10513/X10514/X10517/X10519/ |
| | | X10523/X10524/X10527/X10529/X10538/X10549/X10558/X10568/X11108/ |
| | | X11109/X11110/X11111/X11112/X11113/X11114/X11115/X11116/X11117/X11118/ |
| | | X11119/X11120/X11121/X11122/X11123/X11124/X11125/X11126/X11127/ |
| | | X11128/X11129/X11130/X11131/X11132/X11133/X11135/X11136/X11138/ |
| | | X11140/X11142/X11144/X11147/X11150/X11163/X11174/X11180/X11584/ |
| | | X11585/X11586/X11587/X11588/X11589/X11590/X11591/X11592/X11593/ |
| | | X11594/X11595/X11596/X11597/X11598/X11599/X11600/X11601/X11603/ |
| | | X11605/X11618/X11627/X11897/X11898/X11899/X11900/X11901/X11902/X11903/ |
| | | X11904/X11905/X11906/X11916/X12090/X12091/X12092/X12093/X12200/ |
| | | X1439/X2161/X2163/X2243/X3063/X3064/X3066/X3074/X3148/X3184/ |
| | | X4115/X4116/X4117/X4118/X4125/X4129/X4131/X4133/X4134/X4188/X4190/ |
| | | X4232/X5261/X5262/X5263/X5265/X5266/X5267/X5268/X5269/X5270/X5275/ |
| | | X5277/X5279/X5280/X5284/X5285/X5286/X5287/X5288/X5290/X5292/ |
| | | X5334/X5336/X5338/X5345/X5387/X5388/X6444/X6445/X6446/X6447/X6448/ |
| | | X6449/X6450/X6451/X6453/X6454/X6455/X6456/X6457/X6458/X6459/X6465/ |
| | | X6466/X6467/X6468/X6469/X6471/X6473/X6475/X6477/X6479/X6480/ |
| | | X6481/X6482/X6483/X6484/X6485/X6488/X6489/X6515/X6517/X6518/X6519/ |
| | | X6523/X6524/X6529/X6534/X6567/X6568/X6569/X7590/X7591/X7592/X7594/ |
| | | X7595/X7596/X7597/X7598/X7599/X7600/X7601/X7602/X7603/X7604/ |
| | | X7605/X7606/X7607/X7608/X7609/X7610/X7611/X7612/X7613/X7616/X7619/ |
| | | X7623/X7625/X7626/X7627/X7628/X7629/X7630/X7633/X7634/X7635/X7636/ |
| | | X7637/X7638/X7640/X7642/X7643/X7645/X7646/X7648/X7650/X7666/ |
| | | X7667/X7668/X7669/X7673/X7674/X7676/X7677/X7681/X7686/X7690/X7692/ |
| | | X7720/X7721/X7722/X7723/X8676/X8677/X8678/X8679/X8680/X8681/X8682/ |
| | | X8683/X8684/X8685/X8686/X8687/X8688/X8689/X8690/X8691/X8692/ |
| | | X8693/X8694/X8695/X8696/X8697/X8698/X8699/X8700/X8701/X8702/X8703/ |
| | | X8704/X8705/X8706/X8708/X8710/X8711/X8713/X8717/X8719/X8720/X8722/ |
| | | X8723/X8725/X8727/X8728/X8729/X8730/X8731/X8732/X8735/X8736/ |
| | | X8738/X8739/X8741/X8749/X8750/X8751/X8754/X8755/X8759/X8760/X8764/ |
| | | X8765/X8766/X8769/X8773/X8775/X8778/X8800/X8801/X8802/X8811/X9638/ |
| | | X9639/X9640/X9641/X9642/X9643/X9644/X9645/X9646/X9647/X9648/ |
| | | X9649/X9650/X9651/X9652/X9653/X9654/X9655/X9656/X9657/X9658/X9659/ |
| | | X9660/X9661/X9662/X9663/X9664/X9665/X9666/X9667/X9668/X9669/X9670/ |
| | | X9671/X9673/X9675/X9676/X9678/X9679/X9682/X9683/X9685/X9686/ |
| | | X9688/X9690/X9692/X9693/X9695/X9697/X9699/X9703/X9705/X9706/X9707/ |
| | | X9710/X9714/X9715/X9716/X9719/X9720/X9724/X9727/X9729/X9744/X9745/ |
| | | X9750/X9765 |
| ASN_struct_RSA_{—} | −2.33 | X10513/X10523/X10527/X10529/X10538/X11150/X3184/X4232/X5345/X5387/ |
| RESIDUE_freesasa_het | | X5388/X6524/X6529/X6534/X6567/X6568/X6569/X7667/X7674/X7681/X7686/ |
| | | X7690/X7692/X7720/X7721/X7722/X7723/X8750/X8759/X8765/X8769/ |
| | | X8773/X8775/X8778/X8800/X8801/X8802/X9706/X9715/X9719/X9724/X9727/ |
| | | X9729/X9744/X9745 |
| ASN_seq_aaAll_F | −3.09 | X1854/X4952 |
| ASN_seq_aaUp_G | −3.09 | X191 |
| ASN_seq_aaUp_S | −3.09 | X4132/X5278/X5289/X6470/X6478/X7620/X7639/X8714 |
| ASN_struct_aa_Q | −3.09 | X3787/X4955 |
| ASN_seq_aaAll_D | −3.72 | X4132/X5278/X5289/X6470/X6478/X7620/X7639/X8714 |
| ASN_struct_aa_L | −4.26 | X6194 |
| ASN_struct_RES_{—} | −4.75 | X3747/X4917 |
| DEPTH_msms | | |
| ASN_struct_ASA_{—} | −Inf | X4474 |
| BACKBONE_freesasa_het | | |
| ASN_struct_PHI_dssp | −Inf | X1294/X1295/X1296/X1333/X1334/X1335/X1336/X1337/X1991/X1993/X1995/ |
| | | X1996/X2028/X2029/X2031/X2032/X2033/X2034/X210/X245/X256/X27/X2882/ |
| | | X2885/X2887/X2916/X2918/X2919/X2920/X2921/X31/X3936/X3938/X3962/ |
| | | X3964/X3965/X470/X496/X497/X5096/X5118/X80/X801/X802/X835/X836/ |
| | | X837/X89/X9/X91 |
| ASN_struct_RES_DEPTH_msms | −Inf | X356 |
| ASN_struct_RSA_{—} | −Inf | X4474 |
| BACKBONE_freesasa_het | | |
| ASN_seq_aaAll_A | 1.28 | X393 |
| ASN_seq_aaAll_C | 1.28 | X1844/X2709/X3763/X3865/X83 |
| ASN_seq_aaAll_D | 1.28 | X1199/X1202/X1204/X1876/X1878/X197/X24/X2751/X403/X69/X722/X725/ |
| | | X77 |
| ASN_seq_aaAll_E | 1.28 | X187/X192/X21/X390/X392/X393/X708/X73 |
| ASN_seq_aaAll_F | 1.28 | X1168/X1170/X1175/X1177/X1180/X1182/X1183/X1185/X1199/X1202/X1204/ |
| | | X1355/X1464/X1839/X185/X1850/X1851/X1853/X1856/X1858/X1876/X1878/ |
| | | X190/X197/X2054/X2186/X24/X2704/X2716/X2732/X2733/X2735/X2751/ |
| | | X3117/X3753/X3777/X387/X389/X391/X395/X403/X4940/X699/X701/X702/ |
| | | X706/X709/X71/X711/X722/X725/X77/X853 |
| ASN_seq_aaAll_G | 1.28 | X10462/X10463/X10465/X10469/X10470/X10471/X10472/X10473/X10474/X10483/ |
| | | X10484/X10485/X10495/X11108/X11112/X11117/X11118/X11119/X11585/ |
| | | X1199/X1202/X1204/X1206/X1207/X1439/X1876/X1878/X1883/X197/ |
| | | X198/X2161/X2163/X24/X25/X2736/X2751/X3064/X3066/X3074/X3784/X403/ |
| | | X406/X407/X4116/X4117/X4125/X4129/X4131/X4133/X4134/X4920/X5262/ |
| | | X5265/X5267/X5268/X5269/X5270/X5275/X5277/X5279/X5280/X5284/X5285/ |
| | | X5286/X5287/X5288/X5290/X6445/X6447/X6448/X6449/X6450/X6453/ |
| | | X6454/X6455/X6456/X6457/X6458/X6459/X6465/X6466/X6467/X6468/X6469/ |
| | | X6471/X6475/X6477/X6479/X6480/X6481/X6482/X6483/X6484/X722/X725/ |
| | | X727/X728/X729/X7591/X7594/X7595/X7596/X7597/X7598/X7599/X7600/ |
| | | X7602/X7603/X7604/X7605/X7606/X7607/X7608/X7609/X7610/X7611/X7616/ |
| | | X7619/X7623/X7625/X7626/X7627/X7628/X7629/X7635/X7636/X7637/X7638/ |
| | | X7640/X7643/X77/X78/X8676/X8678/X8680/X8681/X8682/X8683/X8684/ |
| | | X8685/X8686/X8687/X8688/X8689/X8692/X8693/X8694/X8695/X8696/X8697/ |
| | | X8698/X8699/X8700/X8701/X8708/X8710/X8711/X8713/X8717/X8720/ |
| | | X8728/X8729/X8730/X8731/X9639/X9640/X9641/X9643/X9644/X9645/X9646/ |
| | | X9647/X9648/X9649/X9650/X9651/X9652/X9658/X9659/X9660/X9661/X9662/ |
| | | X9663/X9673/X9675/X9676/X9678/X9690 |
| ASN_seq_aaAll_H | 1.28 | X170 |
| ASN_seq_aaAll_I | 1.28 | X170 |
| ASN_seq_aaAll_K | 1.28 | X1179/X1188/X1190/X1192/X1865/X1868/X1894/X19/X2741/X2763/X3/X3802/ |
| | | X398/X713/X716 |
| ASN_seq_aaAll_L | 1.28 | X1199/X1202/X1219/X1355/X1464/X1876/X1899/X197/X2054/X2186/X24/X2771/ |
| | | X3117/X403/X722/X725/X77/X853 |
| ASN_seq_aaAll_M | 1.28 | X49 |
| ASN_seq_aaAll_P | 1.28 | X1199/X1202/X1204/X1274/X1277/X1281/X1287/X1876/X1878/X1961/X197/ |
| | | X1971/X1974/X1978/X239/X24/X2751/X2855/X2857/X2868/X3907/X3909/ |
| | | X403/X462/X5073/X69/X722/X725/X77/X787/X793/X83 |
| ASN_seq_aaAll_Q | 1.28 | X1176/X1179/X1181/X1199/X1202/X1206/X1207/X1208/X1266/X1857/X187/ |
| | | X1876/X1883/X1884/X189/X19/X192/X1952/X197/X198/X21/X24/X25/X2756/ |
| | | X2827/X3/X3865/X388/X390/X392/X394/X396/X403/X406/X407/X70/X700/ |
| | | X705/X707/X708/X710/X722/X725/X727/X728/X729/X73/X77/X78 |
| ASN_seq_aaAll_R | 1.28 | X1274/X170/X1971/X2739/X2857/X3780/X3781/X3787/X4955/X6197 |
| ASN_seq_aaAll_T | 1.28 | X1362/X1363/X1364/X2060/X2065/X2068/X265/X29/X2948/X2961/X3769/X4003/ |
| | | X4935/X4948/X512/X6182/X6187/X7396/X7404/X857/X859/X90 |
| ASN_seq_aaAll_V | 1.28 | X1193/X1832/X1868/X1869/X195/X2705/X2741/X3750/X402/X4928/X715/X719 |
| ASN_seq_aaAll_W | 1.28 | X1174/X1181/X1189/X1194/X16/X183/X1849/X1867/X187/X192/X21/X2743/ |
| | | X3775/X386/X392/X396/X400/X67/X698/X704/X707/X708/X718/X73/X83 |
| ASN_seq_aaDown_A | 1.28 | X104/X110/X1328/X1329/X1330/X1382/X2081/X2083/X234/X235/X255/X2965/ |
| | | X39/X452/X453/X491/X492/X493/X799/X829/X830/X831/X832 |
| ASN_seq_aaDown_C | 1.28 | X105/X1179/X1274/X1277/X1281/X1283/X1284/X1287/X1289/X187/X192/X1971/ |
| | | X1974/X1976/X1978/X1980/X1984/X21/X239/X241/X2857/X2861/X2868/ |
| | | X2870/X390/X392/X3922/X461/X462/X464/X700/X705/X708/X73/X787/ |
| | | X789/X792/X793/X795/X83 |
| ASN_seq_aaDown_D | 1.28 | X1355/X2054/X2729/X853 |
| ASN_seq_aaDown_E | 1.28 | X1167/X1168/X1182/X1835/X1839/X1848/X1850/X1860/X2704/X2723/X2725/ |
| | | X2727/X2732/X2738/X3773/X3774/X3776/X3779/X3782/X387/X4950/X4951/ |
| | | X4956/X6193/X702 |
| ASN_seq_aaDown_G | 1.28 | X1179/X1206/X1207/X1883/X189/X198/X25/X3865/X388/X394/X406/X407/X70/ |
| | | X700/X705/X727/X728/X729/X78 |
| ASN_seq_aaDown_H | 1.28 | X1861/X187/X192/X21/X2728/X3767/X3783/X390/X392/X4933/X4959/X6185/ |
| | | X6196/X708/X73/X7407 |
| ASN_seq_aaDown_K | 1.28 | X19/X3 |
| ASN_seq_aaDown_L | 1.28 | X104/X1355/X1357/X1360/X1464/X170/X2054/X2058/X2059/X2061/X214/X2186/ |
| | | X234/X235/X2945/X2949/X2954/X3116/X3117/X39/X3991/X4004/X452/ |
| | | X510/X5146/X84/X853/X856 |
| ASN_seq_aaDown_M | 1.28 | X110/X1329/X255/X491/X492/X830/X831 |
| ASN_seq_aaDown_N | 1.28 | X1181/X1362/X1363/X1364/X189/X19/X2060/X2064/X2065/X2068/X265/X29/ |
| | | X2948/X2959/X2961/X388/X4003/X4007/X512/X5148/X70/X700/X708/X857/ |
| | | X859/X90 |
| ASN_seq_aaDown_P | 1.28 | X104/X1274/X1289/X1857/X189/X1971/X1976/X234/X235/X2857/X2870/X388/ |
| | | X39/X452/X69/X70/X700/X83 |
| ASN_seq_aaDown_Q | 1.28 | X1176/X1179/X1181/X1857/X187/X189/X19/X192/X21/X3/X388/X390/X392/ |
| | | X394/X396/X70/X700/X705/X707/X708/X710/X73 |
| ASN_seq_aaDown_R | 1.28 | X1189/X1191/X1193/X1194/X1867/X1869/X193/X194/X195/X196/X23/X2743/ |
| | | X397/X399/X400/X401/X402/X714/X715/X717/X718/X719/X75/X76 |
| ASN_seq_aaDown_S | 1.28 | X1191/X1193/X1869/X195/X402/X715/X717/X719 |
| ASN_seq_aaDown_T | 1.28 | X1206/X1207/X1266/X187/X1883/X1952/X198/X21/X25/X2827/X3865/X406/ |
| | | X407/X727/X728/X729/X73/X78 |
| ASN_seq_aaDown_V | 1.28 | X110/X1329/X170/X1832/X255/X2705/X3750/X453/X491/X492/X4928/X831 |
| ASN_seq_aaDown_W | 1.28 | X1/X1166/X1174/X1176/X1179/X16/X183/X1847/X1849/X1857/X189/X19/X2724/ |
| | | X3/X3775/X386/X388/X394/X67/X698/X70/X700/X704/X705/X710 |
| ASN_seq_aaUp_D | 1.28 | X1206/X1207/X1883/X198/X24/X25/X406/X407/X6/X727/X728/X729/X77/X78 |
| ASN_seq_aaUp_E | 1.28 | X1181/X187/X192/X21/X3781/X390/X392/X396/X707/X708/X73 |
| ASN_seq_aaUp_F | 1.28 | X107/X1167/X1168/X1170/X1175/X1177/X1180/X1182/X1183/X1185/X1372/ |
| | | X1835/X1839/X1848/X185/X1850/X1851/X1853/X1856/X1858/X1860/X190/ |
| | | X267/X2704/X2723/X2725/X2727/X2732/X2733/X2735/X3774/X3782/X387/ |
| | | X389/X391/X395/X514/X515/X699/X701/X702/X706/X709/X71/X711/X83/X864/ |
| | | X865 |
| ASN_seq_aaUp_H | 1.28 | X1355/X1464/X2054/X2186/X3117/X853 |
| ASN_seq_aaUp_I | 1.28 | X1184/X1852 |
| ASN_seq_aaUp_K | 1.28 | X1176/X1179/X1188/X1190/X1191/X1192/X1193/X1857/X1865/X1866/X1868/ |
| | | X1869/X187/X189/X1894/X192/X195/X21/X2738/X2741/X2742/X2763/X3779/ |
| | | X3782/X3802/X388/X390/X392/X3935/X394/X398/X402/X4956/X5098/ |
| | | X70/X700/X705/X708/X710/X713/X715/X716/X717/X719/X73 |
| ASN_seq_aaUp_L | 1.28 | X1176/X1179/X1199/X1202/X1204/X1206/X1207/X1208/X1857/X1876/X1878/ |
| | | X1883/X1884/X19/X197/X198/X24/X25/X2751/X2756/X394/X403/X406/X407/ |
| | | X69/X700/X710/X722/X725/X727/X728/X729/X77/X78 |
| ASN_seq_aaUp_M | 1.28 | X1179/X189/X19/X388/X394/X70/X700/X705 |
| ASN_seq_aaUp_P | 1.28 | X1355/X1464/X2054/X2186/X3117/X853 |
| ASN_seq_aaUp_Q | 1.28 | X1206/X1207/X1266/X1883/X1952/X198/X25/X2827/X3865/X390/X406/X407/ |
| | | X708/X727/X728/X729/X78 |
| ASN_seq_aaUp_R | 1.28 | X1274/X1971/X2857/X83 |
| ASN_seq_aaUp_S | 1.28 | X1199/X1202/X1204/X1355/X1876/X1878/X197/X2054/X24/X2751/X403/X722/ |
| | | X725/X77/X853 |
| ASN_seq_aaUp_T | 1.28 | X1168/X1182/X1839/X1850/X1860/X1861/X1866/X2727/X2728/X2732/X2733/ |
| | | X2738/X2742/X3779/X3782/X3783/X3935/X4956/X5098/X702 |
| ASN_seq_aaUp_V | 1.28 | X1179/X1868/X187/X189/X192/X21/X2741/X388/X390/X392/X394/X70/X700/ |
| | | X705/X73 |
| ASN_seq_aaUp_W | 1.28 | X1167/X1168/X1170/X1175/X1177/X1180/X1182/X1183/X1185/X1835/X1839/ |
| | | X1841/X1848/X185/X1850/X1851/X1853/X1856/X1858/X1860/X1861/X190/ |
| | | X2704/X2706/X2715/X2717/X2723/X2725/X2727/X2728/X2732/X2733/X2735/ |
| | | X2738/X2739/X2740/X3747/X3752/X3754/X3760/X3766/X3767/X3773/ |
| | | X3774/X3776/X3779/X3781/X3782/X3783/X3787/X387/X389/X391/X395/X453/ |
| | | X4917/X4921/X4922/X4932/X4933/X4939/X4941/X4950/X4951/X4955/X4956/ |
| | | X4959/X6159/X6162/X6163/X6168/X6184/X6185/X6193/X6196/X6197/ |
| | | X699/X701/X702/X706/X709/X71/X711/X7379/X7387/X7388/X7395/X7407/ |
| | | X8543 |
| ASN_seq_hydrophobicity_kd | 1.28 | X110/X1328/X1329/X1330/X157/X170/X2083/X255/X356/X491/X492/X493/ |
| | | X829/X830/X831/X832 |
| ASN_seq_RSA_accpro20 | 1.28 | X1186/X1854/X238/X3774/X83 |
| ASN_seq_RSA_accproe | 1.28 | X1176/X1355/X2054/X396/X710/X853 |
| ASN_seq_SS_sspro8C | 1.28 | X105/X1176/X1179/X1181/X1206/X1207/X1274/X1277/X1281/X1283/X1284/ |
| | | X1287/X1289/X1857/X187/X1883/X189/X19/X192/X1961/X1971/X1974/X1976/ |
| | | X1978/X198/X1980/X1984/X21/X239/X241/X25/X2855/X2857/X2861/X2868/ |
| | | X2870/X388/X390/X3907/X3909/X392/X3922/X393/X394/X396/X406/ |
| | | X407/X453/X461/X462/X464/X70/X700/X705/X707/X708/X710/X727/X728/ |
| | | X729/X73/X78/X787/X789/X792/X793/X795/X83 |
| ASN_seq_SS_sspro8E | 1.28 | X1188/X1190/X1191/X1192/X1865/X1868/X193/X194/X196/X23/X2741/X397/ |
| | | X398/X399/X401/X713/X714/X716/X717/X75/X76 |
| ASN_seq_SS_sspro8H | 1.28 | X1176/X1179/X1206/X1207/X1843/X1857/X1883/X198/X25/X2708/X3762/X394/ |
| | | X406/X407/X710/X727/X728/X729/X78 |
| ASN_seq_SS_sspro8S | 1.28 | X104/X1177/X1184/X1185/X1852/X1853/X234/X235/X2703/X3745/X39/X452/ |
| | | X706 |
| ASN_seq_SS_sspro8T | 1.28 | X1355/X1832/X2054/X2705/X3750/X853 |
| ASN_seq_SS_ssproE | 1.28 | X1184/X1852 |
| ASN_seq_SS_ssproH | 1.28 | X1176/X1179/X1206/X1207/X1843/X1857/X1883/X198/X25/X2708/X3762/X394/ |
| | | X406/X407/X4920/X710/X727/X728/X729/X78 |
| ASN_struct_aa_A | 1.28 | X1206/X1207/X1358/X1361/X1365/X1883/X198/X2056/X2062/X2066/X2067/ |
| | | X2069/X25/X2950/X2952/X2955/X2956/X2958/X2960/X2962/X3865/X3994/ |
| | | X3997/X4000/X4001/X4005/X4008/X4009/X406/X407/X511/X5143/X5150/X5152/ |
| | | X5153/X5155/X6347/X6350/X6351/X727/X728/X729/X7518/X78/X854/ |
| | | X858 |
| ASN_struct_aa_C | 1.28 | X1362/X1363/X1364/X2060/X2064/X2065/X2068/X265/X29/X2948/X2959/X2961/ |
| | | X4003/X4007/X512/X5148/X857/X859/X90 |
| ASN_struct_aa_E | 1.28 | X1163/X1171/X1836/X1842/X2707/X383/X697 |
| ASN_struct_aa_F | 1.28 | X1177/X1182/X1199/X1202/X1850/X1860/X1876/X190/X197/X24/X2727/X2732/ |
| | | X2738/X3779/X3782/X387/X389/X395/X403/X4956/X706/X71/X711/X722/ |
| | | X725/X7394/X77 |
| ASN_struct_aa_G | 1.28 | X799 |
| ASN_struct_aa_H | 1.28 | X117/X13/X1354/X263/X45/X508/X509/X851/X852 |
| ASN_struct_aa_K | 1.28 | X19/X3/X3865 |
| ASN_struct_aa_L | 1.28 | X10462/X10463/X10465/X10469/X10470/X10471/X10472/X10473/X10474/X10483/ |
| | | X10484/X10485/X10495/X11108/X11112/X11117/X11118/X11119/X11585/ |
| | | X1439/X2161/X2163/X3064/X3066/X3074/X4116/X4117/X4125/X4129/ |
| | | X4131/X4133/X4134/X5262/X5265/X5267/X5268/X5269/X5270/X5275/X5277/ |
| | | X5279/X5280/X5284/X5285/X5286/X5287/X5288/X5290/X6445/X6447/X6448/ |
| | | X6449/X6450/X6453/X6454/X6455/X6456/X6457/X6458/X6459/X6465/ |
| | | X6466/X6467/X6468/X6469/X6471/X6475/X6477/X6479/X6480/X6481/X6482/ |
| | | X6483/X6484/X7591/X7594/X7595/X7596/X7597/X7598/X7599/X7600/X7602/ |
| | | X7603/X7604/X7605/X7606/X7607/X7608/X7609/X7610/X7611/X7616/ |
| | | X7619/X7623/X7625/X7626/X7627/X7628/X7629/X7635/X7636/X7637/X7638/ |
| | | X7640/X7643/X8676/X8678/X8680/X8681/X8682/X8683/X8684/X8685/X8686/ |
| | | X8687/X8688/X8689/X8692/X8693/X8694/X8695/X8696/X8697/X8698/ |
| | | X8699/X8700/X8701/X8708/X8710/X8711/X8713/X8717/X8720/X8728/X8729/ |
| | | X8730/X8731/X9639/X9640/X9641/X9643/X9644/X9645/X9646/X9647/X9648/ |
| | | X9649/X9650/X9651/X9652/X9658/X9659/X9660/X9661/X9662/X9663/ |
| | | X9673/X9675/X9676/X9678/X9690 |
| ASN_struct_aa_P | 1.28 | X1168/X1182/X1274/X1839/X1850/X1860/X1971/X2727/X2732/X2738/X2857/ |
| | | X3779/X3782/X387/X49/X4956/X702/X83 |
| ASN_struct_aa_Q | 1.28 | X1179/X187/X189/X21/X388/X390/X394/X70/X700/X705/X73 |
| ASN_struct_aa_R | 1.28 | X1168/X1170/X1175/X1177/X1182/X1183/X1185/X1839/X185/X1850/X1851/ |
| | | X1853/X1856/X1860/X190/X2704/X2727/X2732/X2733/X2735/X3773/X3782/ |
| | | X387/X389/X391/X395/X4950/X4951/X699/X702/X706/X709/X71/X711 |
| ASN_struct_aa_S | 1.28 | X10462/X10463/X10465/X10469/X10470/X10471/X10472/X10473/X10474/X10483/ |
| | | X10484/X10485/X10494/X10495/X10496/X10499/X10501/X10503/X10505/ |
| | | X10508/X10510/X11108/X11112/X11117/X11118/X11119/X11134/X11137/ |
| | | X11139/X11585/X11602/X1356/X1357/X1359/X1360/X1439/X1441/X2055/ |
| | | X2057/X2058/X2059/X2061/X2063/X2120/X214/X2161/X2163/X2164/X2165/ |
| | | X2944/X2945/X2946/X2947/X2949/X2951/X2953/X2954/X2957/X2996/ |
| | | X2997/X3002/X3004/X3064/X3066/X3068/X3069/X3071/X3074/X3075/X3991/ |
| | | X3992/X3993/X3995/X3996/X3998/X3999/X4002/X4004/X4006/X4025/X4026/ |
| | | X4032/X4033/X4037/X4038/X4039/X4040/X4042/X4116/X4117/X4122/ |
| | | X4125/X4126/X4128/X4129/X4131/X4132/X4133/X4134/X4136/X4137/X4138/ |
| | | X510/X5141/X5142/X5144/X5145/X5146/X5147/X5149/X5151/X5154/X5161/ |
| | | X5164/X5165/X5169/X5170/X5171/X5172/X5173/X5174/X5178/X5179/X5180/ |
| | | X5181/X5262/X5265/X5267/X5268/X5269/X5270/X5272/X5274/X5275/ |
| | | X5277/X5278/X5279/X5280/X5281/X5284/X5285/X5286/X5287/X5288/X5289/ |
| | | X5290/X5291/X5293/X5297/X5298/X5299/X5300/X5301/X5302/X6344/X6345/ |
| | | X6346/X6348/X6349/X6352/X6353/X6355/X6356/X6357/X6358/X6359/ |
| | | X6363/X6364/X6365/X6366/X6367/X6368/X6369/X6445/X6447/X6448/X6449/ |
| | | X6450/X6453/X6454/X6455/X6456/X6457/X6458/X6459/X6460/X6462/X6465/ |
| | | X6466/X6467/X6468/X6469/X6470/X6471/X6472/X6474/X6475/X6477/ |
| | | X6478/X6479/X6480/X6481/X6482/X6483/X6484/X6486/X6487/X6490/X6491/ |
| | | X6495/X6496/X6497/X6498/X6499/X6500/X6501/X6502/X7517/X7519/X7520/ |
| | | X7521/X7522/X7524/X7525/X7526/X7527/X7528/X7529/X7530/X7591/ |
| | | X7594/X7595/X7596/X7597/X7598/X7599/X7600/X7602/X7603/X7604/X7605/ |
| | | X7606/X7607/X7608/X7609/X7610/X7611/X7614/X7615/X7616/X7618/X7619/ |
| | | X7620/X7621/X7623/X7625/X7626/X7627/X7628/X7629/X7631/X7632/ |
| | | X7635/X7636/X7637/X7638/X7639/X7640/X7641/X7643/X7644/X7647/X7649/ |
| | | X7651/X7652/X7654/X7655/X7656/X7657/X7658/X7659/X7660/X7661/X84/ |
| | | X855/X856/X8633/X8634/X8635/X8636/X8676/X8678/X8680/X8681/X8682/ |
| | | X8683/X8684/X8685/X8686/X8687/X8688/X8689/X8692/X8693/X8694/X8695/ |
| | | X8696/X8697/X8698/X8699/X8700/X8701/X8707/X8708/X8709/X8710/ |
| | | X8711/X8712/X8713/X8714/X8715/X8716/X8717/X8718/X8720/X8721/X8724/ |
| | | X8726/X8728/X8729/X8730/X8731/X8733/X8734/X8737/X8740/X8742/X8743/ |
| | | X8744/X8745/X8746/X8747/X8748/X9639/X9640/X9641/X9643/X9644/ |
| | | X9645/X9646/X9647/X9648/X9649/X9650/X9651/X9652/X9658/X9659/X9660/ |
| | | X9661/X9662/X9663/X9672/X9673/X9674/X9675/X9676/X9677/X9678/X9680/ |
| | | X9681/X9684/X9687/X9689/X9690/X9691/X9694/X9696/X9698/X9700/ |
| | | X9701/X9702 |
| ASN_struct_aa_T | 1.28 | X1189/X1191/X1194/X1866/X1867/X193/X194/X195/X196/X23/X2742/X2743/ |
| | | X3935/X397/X399/X400/X401/X402/X5098/X714/X715/X717/X718/X75/X76 |
| ASN_struct_aa_V | 1.28 | X187/X192/X21/X390/X392/X73 |
| ASN_struct_aa_W | 1.28 | X105/X1167/X1168/X1175/X1176/X1179/X1181/X1182/X1183/X1185/X1283/ |
| | | X1284/X1355/X1835/X1839/X1848/X185/X1850/X1851/X1853/X1856/X1857/ |
| | | X1860/X1861/X187/X189/X19/X192/X1980/X2054/X21/X241/X2704/X2723/ |
| | | X2725/X2727/X2728/X2732/X2733/X2735/X2738/X3/X3773/X3774/X3776/ |
| | | X3779/X3782/X3783/X387/X388/X390/X391/X392/X394/X396/X461/X464/X4950/ |
| | | X4951/X4956/X6193/X699/X70/X700/X702/X705/X707/X708/X709/X710/ |
| | | X73/X789/X792/X795/X83/X853 |
| ASN_struct_aa_Y | 1.28 | X1199/X1202/X1876/X197/X24/X403/X722/X725/X77 |
| ASN_struct_ASA_dssp | 1.28 | X238/X83 |
| ASN_struct_ASA_{—} | 1.28 | X2049/X2940/X3865/X3938/X3987/X5470/X83 |
| ALL_freesasa_het | | |
| ASN_struct_ASA_{—} | 1.28 | X170/X2018/X2902/X3956/X5109 |
| BACKBONE_freesasa_het | | |
| ASN_struct_ASA_{—} | 1.28 | X115/X12/X1350/X1351/X1352/X1529/X157/X170/X2050/X2051/X2285/X2287/ |
| NONPOLAR_freesasa_het | | X260/X2941/X3226/X4295/X44/X504/X83/X846/X847 |
| ASN_struct_ASA_{—} | 1.28 | X238/X83 |
| POLAR_freesasa_het | | |
| ASN_struct_ASA_{—} | 1.28 | X1267/X1277/X1281/X1287/X157/X1953/X1974/X1978/X238/X239/X2828/X2868/ |
| RESIDUE_freesasa_het | | X3865/X462/X787/X793/X83 |
| ASN_struct_CA_{—} | 1.28 | X1177/X157/X3747/X4917/X711 |
| DEPTH_msms | | |
| ASN_struct_PHI_dssp | 1.28 | X1366/X1527/X1528/X2070/X2231/X2232/X2283/X3131/X3132/X3133/X4174/ |
| | | X4176/X568/X965/X966 |
| ASN_struct_PSI_dssp | | X10462/X10463/X10464/X10465/X10466/X10467/X10468/X10469/X10470/X10471/ |
| | | X10472/X10473/X10474/X10475/X10476/X10477/X10478/X10479/X10480/ |
| | | X10481/X10482/X10483/X10484/X10485/X10486/X10487/X10488/X10489/ |
| | | X10490/X10491/X10492/X10493/X10494/X10495/X10496/X10497/X10498/ |
| | | X10499/X10500/X10501/X10502/X10503/X10504/X10505/X10506/X10507/ |
| | | X10508/X10509/X10510/X10512/X10514/X10517/X10519/X10524/X10549/ |
| | | X10558/X10568/X11108/X11109/X11110/X11111/X11112/X11113/X11114/X11115/ |
| | | X11116/X11117/X11118/X11119/X11120/X11121/X11122/X11123/X11124/ |
| | | X11125/X11126/X11127/X11128/X11129/X11130/X11131/X11132/X11133/ |
| | | X11134/X11135/X11136/X11137/X11138/X11139/X11140/X11142/X11144/ |
| | | X11147/X11163/X11174/X11180/X11584/X11585/X11586/X11587/X11588/ |
| | | X11589/X11590/X11591/X11592/X11593/X11594/X11595/X11596/X11597/ |
| | | X11598/X11599/X11600/X11601/X11602/X11603/X11605/X11618/X11627/X11897/ |
| | | X11898/X11899/X11900/X11901/X11902/X11903/X11904/X11905/X11906/ |
| | | X11916/X1194/X12090/X12091/X12092/X12093/X12200/X1357/X1360/ |
| | | X1439/X1440/X1441/X1867/X1869/X195/X2058/X2059/X2061/X2120/X214/ |
| | | X2161/X2162/X2163/X2164/X2165/X2166/X2243/X238/X254/X2743/X2945/ |
| | | X2947/X2949/X2954/X2996/X2997/X3002/X3004/X3063/X3064/X3065/X3066/ |
| | | X3068/X3069/X3070/X3071/X3073/X3074/X3075/X3148/X3991/X3999/X4004/ |
| | | X402/X4025/X4026/X4032/X4033/X4037/X4038/X4039/X4040/X4042/X4114/ |
| | | X4115/X4116/X4117/X4118/X4119/X4122/X4124/X4125/X4126/X4127/ |
| | | X4128/X4129/X4130/X4131/X4133/X4134/X4136/X4137/X4138/X4188/X4190/ |
| | | X489/X510/X5146/X5149/X5161/X5164/X5165/X5169/X5170/X5171/X5172/ |
| | | X5173/X5174/X5178/X5179/X5180/X5181/X5261/X5262/X5263/X5264/X5265/ |
| | | X5266/X5267/X5268/X5269/X5270/X5272/X5273/X5274/X5275/X5276/ |
| | | X5277/X5279/X5280/X5281/X5283/X5284/X5285/X5286/X5287/X5288/X5290/ |
| | | X5291/X5292/X5293/X5297/X5298/X5299/X5300/X5301/X5302/X5334/X5336/ |
| | | X5338/X6/X6345/X6353/X6355/X6356/X6357/X6358/X6359/X6363/X6364/ |
| | | X6365/X6366/X6367/X6368/X6369/X6444/X6445/X6446/X6447/X6448/X6449/ |
| | | X6450/X6451/X6452/X6453/X6454/X6455/X6456/X6457/X6458/X6459/ |
| | | X6460/X6462/X6463/X6465/X6466/X6467/X6468/X6469/X6471/X6472/X6473/ |
| | | X6474/X6475/X6476/X6477/X6479/X6480/X6481/X6482/X6483/X6484/X6485/ |
| | | X6486/X6487/X6488/X6489/X6490/X6491/X6495/X6496/X6497/X6498/ |
| | | X6499/X6500/X6501/X6502/X6515/X6517/X6518/X6519/X6523/X715/X7521/ |
| | | X7522/X7524/X7525/X7526/X7527/X7528/X7529/X7530/X7590/X7591/X7592/ |
| | | X7593/X7594/X7595/X7596/X7597/X7598/X7599/X7600/X7601/X7602/X7603/ |
| | | X7604/X7605/X7606/X7607/X7608/X7609/X7610/X7611/X7612/X7613/ |
| | | X7614/X7615/X7616/X7617/X7618/X7619/X7621/X7623/X7625/X7626/X7627/ |
| | | X7628/X7629/X7630/X7631/X7632/X7633/X7634/X7635/X7636/X7637/X7638/ |
| | | X7640/X7641/X7642/X7643/X7644/X7645/X7646/X7647/X7648/X7649/ |
| | | X7650/X7651/X7652/X7654/X7655/X7656/X7657/X7658/X7659/X7660/X7661/ |
| | | X7666/X7668/X7669/X7673/X7676/X7677/X84/X856/X8633/X8634/X8635/ |
| | | X8636/X8676/X8677/X8678/X8679/X8680/X8681/X8682/X8683/X8684/X8685/ |
| | | X8686/X8687/X8688/X8689/X8690/X8691/X8692/X8693/X8694/X8695/X8696/ |
| | | X8697/X8698/X8699/X8700/X8701/X8702/X8703/X8704/X8705/X8706/ |
| | | X8707/X8708/X8709/X8710/X8711/X8712/X8713/X8715/X8717/X8718/X8719/ |
| | | X8720/X8721/X8722/X8723/X8724/X8725/X8726/X8727/X8728/X8729/X8730/ |
| | | X8731/X8732/X8733/X8734/X8735/X8736/X8737/X8738/X8739/X8740/ |
| | | X8741/X8742/X8743/X8744/X8745/X8746/X8747/X8748/X8749/X8751/X8754/ |
| | | X8755/X8760/X8764/X8766/X8811/X907/X9638/X9639/X9640/X9641/X9642/ |
| | | X9643/X9644/X9645/X9646/X9647/X9648/X9649/X9650/X9651/X9652/X9653/ |
| | | X9654/X9655/X9656/X9657/X9658/X9659/X9660/X9661/X9662/X9663/ |
| | | X9664/X9665/X9666/X9667/X9668/X9669/X9670/X9671/X9672/X9673/X9674/ |
| | | X9675/X9676/X9677/X9678/X9679/X9680/X9681/X9682/X9683/X9684/X9685/ |
| | | X9686/X9687/X9688/X9689/X9690/X9691/X9692/X9693/X9694/X9695/ |
| | | X9696/X9697/X9698/X9699/X9700/X9701/X9702/X9703/X9705/X9707/X9710/ |
| | | X9714/X9716/X9720/X9750/X9765 |
| ASN_struct_RES_{—} | 1.28 | X1195/X1198/X1870/X1873/X1875/X2745/X2747/X2761/X3790/X3799/X3801/ |
| DEPTH_msms | | X3938/X49/X4966/X4967/X7073/X711/X720 |
| ASN_struct_RSA_dssp | 1.28 | X2064/X238/X2703/X2959/X3745/X4007/X5148/X83 |
| ASN_struct_RSA_{—} | 1.28 | X2049/X2064/X2940/X2959/X3865/X3938/X3987/X4007/X5148/X5470/X83 |
| ALL_freesasa_het | | |
| ASN_struct_RSA | 1.28 | X170/X2018/X2902/X3956/X5109 |
| BACKBONE_freesasa_het | | |
| ASN_struct_RSA_{—} | 1.28 | X115/X12/X1350/X1351/X1352/X1529/X157/X170/X2050/X2051/X2285/X2287/ |
| NONPOLAR_freesasa_het | | X260/X2941/X3226/X4295/X44/X504/X83/X846/X847 |
| ASN_struct_RSA_{—} | 1.28 | X238/X83 |
| POLAR_freesasa_het | | |
| ASN_struct_RSA_{—} | 1.28 | X1267/X1277/X1281/X1287/X157/X1953/X1974/X1978/X238/X239/X2828/X2868/ |
| RESIDUE_freesasa_het | | X3865/X462/X787/X793/X83 |
| ASN_struct_SS_dsspH | 1.28 | X1176/X1181/X1206/X1857/X189/X198/X25/X2703/X3745/X3775/X388/X394/ |
| | | X406/X407/X70/X710/X727/X728/X78/X799 |
| ASN_struct_SS_dsspT | 1.28 | X1168/X1170/X1175/X1177/X1180/X1182/X1183/X1185/X1835/X1839/X185/ |
| | | X1850/X1851/X1853/X1856/X1858/X1860/X1861/X190/X2704/X2727/X2728/ |
| | | X2732/X2733/X2735/X2738/X3773/X3774/X3779/X3782/X3783/X387/X389/ |
| | | X391/X395/X4922/X4950/X4951/X4956/X6159/X6163/X699/X701/X702/X706/ |
| | | X709/X71/X711/X7379/X7388/X8543 |
| SER.THR_seq_aaUp_P | 1.28 | X1 |
| SER.THR_seq_aaUp_Q | 1.28 | X1 |
| SER.THR_seq_hydrophobicity_kd | 1.28 | X2101/X2111/X380/X381/X495 |
| SER.THR_struct_aa_E | 1.28 | X208/X86 |
| SER.THR_struct_RES_{—} | 1.28 | X19/X2/X204/X28/X81/X87 |
| DEPTH_msms | | |
| ASN_seq_aaAll_C | 2.33 | X1163/X1171/X1834/X1836/X1842/X2707/X2712/X3761/X383/X697/X8555 |
| ASN_seq_aaAll_G | 2.33 | X10427/X1186/X8544/X9568 |
| ASN_seq_aaAll_K | 2.33 | X7394 |
| ASN_seq_aaAll_Q | 2.33 | X2736 |
| ASN_seq_aaAll_T | 2.33 | X10427/X8544/X9568 |
| ASN_seq_aaDown_C | 2.33 | X1163/X1171/X1834/X1836/X1842/X2707/X2712/X3761/X383/X697 |
| ASN_seq_aaDown_G | 2.33 | X6 |
| ASN_seq_aaDown_Q | 2.33 | X3780 |
| ASN_seq_aaDown_S | 2.33 | X4947/X6181/X7403 |
| ASN_seq_aaUp_C | 2.33 | X1163/X1171/X1834/X1836/X1842/X2707/X2712/X3761/X383/X697 |
| ASN_seq_aaUp_I | 2.33 | X8560 |
| ASN_seq_aaUp_K | 2.33 | X1167/X1848/X2723/X2725 |
| ASN_seq_aaUp_V | 2.33 | X1199/X1202/X1204/X1876/X1878/X197/X24/X2751/X403/X722/X725/X7382/ |
| | | X77 |
| ASN_seq_SS_sspro8C | 2.33 | X1862 |
| ASN_seq_SS_sspro8S | 2.33 | X1317/X1320/X1321/X2012/X2021/X2023/X253/X2910/X2913/X3958/X487/ |
| | | X823/X824 |
| ASN_struct_aa_T | 2.33 | X1479/X2212/X3116 |
| ASN_struct_aa_V | 2.33 | X117/X13/X1354/X1355/X1464/X2054/X2186/X2211/X263/X3115/X3117/X4166/ |
| | | X45/X508/X509/X7406/X851/X852/X853 |
| ASN_struct_ASA_dssp | 2.33 | X170 |
| ASN_struct_ASA_{—} | 2.33 | X170 |
| ALL_freesasa_het | | |
| ASN_struct_ASA_{—} | 2.33 | X170 |
| POLAR_freesasa_het | | |
| ASN_struct_ASA_{—} | 2.33 | X170 |
| RESIDUE_freesasa_het | | |
| ASN_struct_CA_D | 2.33 | X1536/X1538/X1539/X1567/X2297/X2298/X2299/X2301/X2302/X2303/X2343/ |
| EPTH_msms | | X3225/X3228/X3229/X3230/X3232/X3233/X3235/X3237/X3238/X3239/X3304/ |
| | | X3305/X3306/X4293/X4294/X4297/X4299/X4300/X4302/X4303/X4304/ |
| | | X4306/X4308/X4310/X4311/X4403/X4404/X4405/X4406/X4408/X5468/X5469/ |
| | | X5471/X5474/X5477/X5480/X5481/X5486/X5492/X5493/X5494/X5615/X5616/ |
| | | X5618/X5619/X5620/X5621/X5623/X570/X6669/X6672/X6673/X6679/X6684/ |
| | | X6686/X6688/X6689/X6833/X6834/X6835/X6836/X6838/X6839/X6841/ |
| | | X7832/X7838/X7843/X7844/X8002/X8003/X8005/X8006/X8008/X8912/X9072/ |
| | | X9073/X9075/X970/X971 |
| ASN_struct_RES_{—} | 2.33 | X4474 |
| DEPTH_msms | | |
| ASN_struct_RSA_dssp | 2.33 | X170 |
| ASN_struct_RSA_{—} | 2.33 | X170 |
| ALL_freesasa_het | | |
| ASN_struct_RSA_{—} | 2.33 | X10496/X10499/X10501/X10503/X10505/X10508/X10510/X11134/X11137/X11139/ |
| POLAR_freesasa_het | | X11602/X1441/X170/X2164/X2165/X3068/X3069/X3071/X3075/X4122/ |
| | | X4126/X4128/X4136/X4137/X4138/X5272/X5274/X5281/X5291/X5297/X5298/ |
| | | X5299/X5300/X5301/X5302/X6460/X6472/X6474/X6486/X6487/X6495/ |
| | | X6496/X6497/X6498/X6499/X6500/X6501/X6502/X7614/X7631/X7632/X7641/ |
| | | X7644/X7647/X7649/X7654/X7655/X7656/X7657/X7658/X7659/X7660/X7661/ |
| | | X8718/X8721/X8724/X8726/X8733/X8734/X8737/X8740/X8743/X8744/ |
| | | X8745/X8746/X8747/X8748/X9680/X9681/X9684/X9687/X9691/X9694/X9696/ |
| | | X9698/X9700/X9701/X9702 |
| ASN_struct_RSA_{—} | 2.33 | X170 |
| RESIDUE_freesasa_het | | |
| ASN_struct_SS_dsspT | 2.33 | X4921/X6162/X7387 |
| ASN_seq_aaAll_K | 3.09 | X389 |
| ASN_seq_aaUp_V | 3.09 | X1880/X2753/X3794 |
| ASN_struct_aa_K | | |
| | 3.09 | X49 |
| ASN_struct_aa_Q | 3.09 | X2717/X3754 |
| ASN_struct_aa_T | 3.09 | X7396 |
| ASN_struct_ASA_dssp | 3.09 | X5096 |
| ASN_struct_PHI_dssp | 3.09 | X1529/X2285/X3207 |
| ASN_struct_RSA_dssp | 3.09 | X5096 |
| ASN_struct_SS_dsspE | 3.09 | X1163/X1171/X1836/X1842/X2707/X383/X697 |
| ASN_struct_aa_F | 3.72 | X8545 |
| ASN_seq_RSA_accpro20 | 4.26 | X356 |
| ASN_seq_SS_sspro8E | 4.75 | X1355/X2054/X853 |
| ASN_seq_SS_ssproE | 4.75 | X1355/X2054/X853 |
| ASN_struct_ASA_{—} | 4.75 | X356 |
| BACKBONE_freesasa_het | | |
| ASN_struct_RSA_{—} | 4.75 | X356 |
| BACKBONE_freesasa_het | | |
| ASN_struct_ASA_{—} | Inf | X356 |
| NONPOLAR_freesasa_het | | |
| ASN_struct_RSA_{—} | Inf | X356 |
| NONPOLAR_freesasa_het | | |

### Databases

Methods and systems described herein may comprise one or more databases. The methods and systems described herein may comprise at least about 1, 2, 3, 4, 5, 6, 7, 8, 9, 10, 11, 12, 13, 14, 15, 16, 17, 18, 19, 20, 30, or more databases. The databases may comprise genomic, proteomic, glycomic, biological (e.g., protein sequence databases, protein structure databases, protein model databases, nucleic acid sequence databases, nucleic acid structure databases, nucleic acid model databases), biomedical, or scientific databases. The databases may comprise publicly available databases. Alternatively or additionally, the databases may comprise proprietary databases. The databases may comprise commercially available databases. The databases may include, but are not limited to, UniCarbKB, GlyConnect, and the Protein Data Bank (PDB).

Databases as described herein may comprise one or more sequences. The sequences may comprise reference or variant sequences. Databases as described herein may comprise one or more glycosylation features. Databases as described herein may comprise one or more measures of association between sequences or parts thereof (e.g., glycosites, amino acids structurally and/or sequence proximal to glycosites) and glycosylation features. The one or more measures of association may comprise intermolecular relations (IMRs) as described elsewhere herein.

The methods disclosed herein may comprise analyzing information contained in one or more databases. The method disclosed herein may comprise analyzing information contained in at least about 1, 2, 3, 4, 5, 6, 7, 8, 9, 10, 11, 12, 13, 14, 15, 16, 17, 18, 19, 20, 30, or more databases. Analyzing the information in the one or more databases may comprise one or more algorithms (e.g., trained algorithms), computers, processors, memory locations, devices, or a combination thereof.

### Trained Algorithms

Methods and systems as described herein may employ one or more trained algorithms. The trained algorithm(s) may process or operate on one or more datasets comprising information about biomolecules (e.g., biomolecular features), glycans and glycosylation features, or any combination thereof. In some embodiments, the glycan embeddings (e.g., glycompare, sweetnet) that go into the algorithms that generate the IMRs are derived from empirical observations. In some embodiments, the datasets comprise structural or sequence information about biomolecules. In some embodiments, the datasets comprise one or more datasets of glycosylation features. The one or more datasets may be observed empirically, derived from computational studies, be derived from or contained in one or more databases, or any combination thereof.

The trained algorithm may comprise an unsupervised machine learning algorithm. The trained algorithm may comprise a supervised machine learning algorithm. The trained algorithm may comprise a semi-supervised machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise a self-supervised machine learning algorithm.

In some embodiments, a machine learning algorithm (or software module) of a platform as described herein utilizes one or more neural networks. In some embodiments, a neural network is a type of computational system that can leam the relationships between an input dataset and a target dataset. A neural network may be a software representation of a human neural system (e.g. cognitive system), intended to capture “learning” and “generalization” abilities as used by a human. In some embodiments, the machine learning algorithm (or software module) comprises a neural network comprising a CNN. Non-limiting examples of structural components of embodiments of the machine learning software described herein include: CNNs, recurrent neural networks, dilated CNNs, fully-connected neural networks, deep generative models, recurrent neural networks (RNNs), RNNs using long short-term memory (LSTM) units, and Boltzmann machines.

In some embodiments, a neural network comprises a series of layers termed “neurons.” In some embodiments, a neural network comprises an input layer, to which data is presented; one or more internal, and/or “hidden”, layers; and an output layer. A neuron may be connected to neurons in other layers via connections that have weights, which are parameters that control the strength of the connection. The number of neurons in each layer may be related to the complexity of the problem to be solved. The minimum number of neurons required in a layer may be determined by the problem complexity, and the maximum number may be limited by the ability of the neural network to generalize. The input neurons may receive data being presented and then transmit that data to the first hidden layer through connections' weights, which are modified during training. The first hidden layer may process the data and transmit its result to the next layer through a second set of weighted connections. Each subsequent layer may “pool” the results from the previous layers into more complex relationships. In addition, whereas conventional software programs require writing specific instructions to perform a function, neural networks are programmed by training them with a known sample set and allowing them to modify themselves during (and after) training so as to provide a desired output such as an output value. After training, when a neural network is presented with new input data, it is configured to generalize what was “learned” during training and apply what was learned from training to the new previously unseen input data in order to generate an output associated with that input.

In some embodiments, the neural network comprises ANNs. ANN may be machine learning algorithms that may be trained to map an input dataset to an output dataset, where the ANN comprises an interconnected group of nodes organized into multiple layers of nodes. For example, the ANN architecture may comprise at least an input layer, one or more hidden layers, and an output layer. The ANN may comprise any total number of layers, and any number of hidden layers, where the hidden layers function as trainable feature extractors that allow mapping of a set of input data to an output value or set of output values. As used herein, a deep learning algorithm (such as a DNN) is an ANN comprising a plurality of hidden layers, e.g., two or more hidden layers. Each layer of the neural network may comprise a number of nodes (or “neurons”). A node receives input that comes either directly from the input data or the output of nodes in previous layers, and performs a specific operation, e.g., a summation operation. A connection from an input to a node is associated with a weight (or weighting factor). The node may sum up the products of all pairs of inputs and their associated weights. The weighted sum may be offset with a bias. The output of a node or neuron may be gated using a threshold or activation function. The activation function may be a linear or non-linear function. The activation function may be, for example, a rectified linear unit (ReLU) activation function, a Leaky ReLU activation function, or other function such as a saturating hyperbolic tangent, identity, binary step, logistic, arctan, softsign, parametric rectified linear unit, exponential linear unit, softplus, bent identity, softexponential, sinusoid, sinc, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training dataset and a gradient descent or backward propagation method so that the output value(s) that the ANN computes are consistent with the examples included in the training dataset.

The number of nodes used in the input layer of the ANN or DNN may be at least about 10, 50, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, or greater. In some instances, the number of node used in the input layer may be at most about 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 50, 10, or less. In some instances, the total number of layers used in the ANN or DNN (including input and output layers) may be at least about 3, 4, 5, 10, 15, 20, or greater. In some instances, the total number of layers may be at most about 20, 15, 10, 5, 4, 3, or less.

In some instances, the total number of learnable or trainable parameters, e.g., weighting factors, biases, or threshold values, used in the ANN or DNN may be at least about 10, 50, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, or greater. In some instances, the number of learnable parameters may be at most about 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 50, 10, or less.

In some embodiments of a machine learning software module as described herein, a machine learning software module comprises a neural network such as a deep CNN. In some embodiments in which a CNN is used, the network is constructed with any number of convolutional layers, dilated layers or fully-connected layers. In some embodiments, the number of convolutional layers is between 1-10 and the dilated layers between 0-10. The total number of convolutional layers (including input and output layers) may be at least about 1, 2, 3, 4, 5, 10, 15, 20, or greater, and the total number of dilated layers may be at least about 1, 2, 3, 4, 5, 10, 15, 20, or greater. The total number of convolutional layers may be at most about 20, 15, 10, 5, 4, 3, or less, and the total number of dilated layers may be at most about 20, 15, 10, 5, 4, 3, or less. In some embodiments, the number of convolutional layers is between 1-10 and the fully-connected layers between 0-10. The total number of convolutional layers (including input and output layers) may be at least about 1, 2, 3, 4, 5, 10, 15, 20, or greater, and the total number of fully-connected layers may be at least about 1, 2, 3, 4, 5, 10, 15, 20, or greater. The total number of convolutional layers may be at most about 20, 15, 10, 5, 4, 3, 2, 1, or less, and the total number of fully-connected layers may be at most about 20, 15, 10, 5, 4, 3, 2, 1, or less.

In some embodiments, the input data for training of the ANN may comprise a variety of input values depending whether the machine learning algorithm is used for processing sequence or structural data. In general, the ANN or deep learning algorithm may be trained using one or more training datasets comprising the same or different sets of input and paired output data.

In some embodiments, a machine learning software module comprises a neural network comprising a CNN, RNN, dilated CNN, fully-connected neural networks, deep generative models and deep restricted Boltzmann machines.

In some embodiments, a machine learning algorithm comprises CNNs. The CNN may be deep and feedforward ANNs. The CNN may be applicable to analyzing visual imagery. The CNN may comprise an input, an output layer, and multiple hidden layers. The hidden layers of a CNN may comprise convolutional layers, pooling layers, fully-connected layers and normalization layers. The layers may be organized in 3 dimensions: width, height and depth.

The convolutional layers may apply a convolution operation to the input and pass results of the convolution operation to the next layer. For processing images, the convolution operation may reduce the number of free parameters, allowing the network to be deeper with fewer parameters. In neural networks, each neuron may receive input from some number of locations in the previous layer. In a convolutional layer, neurons may receive input from only a restricted subarea of the previous layer. The convolutional layer's parameters may comprise a set of learnable filters (or kernels). The learnable filters may have a small receptive field and extend through the full depth of the input volume. During the forward pass, each filter may be convolved across the width and height of the input volume, compute the dot product between the entries of the filter and the input, and produce a two-dimensional activation map of that filter. As a result, the network may learn filters that activate when it detects some specific type of feature at some spatial position in the input.

In some embodiments, the pooling layers comprise global pooling layers. The global pooling layers may combine the outputs of neuron clusters at one layer into a single neuron in the next layer. For example, max pooling layers may use the maximum value from each of a cluster of neurons in the prior layer; and average pooling layers may use the average value from each of a cluster of neurons at the prior layer.

In some embodiments, the fully-connected layers connect every neuron in one layer to every neuron in another layer. In neural networks, each neuron may receive input from some number locations in the previous layer. In a fully-connected layer, each neuron may receive input from every element of the previous layer.

In some embodiments, the normalization layer is a batch normalization layer. The batch normalization layer may improve the performance and stability of neural networks. The batch normalization layer may provide any layer in a neural network with inputs that are zero mean/unit variance. The advantages of using batch normalization layer may include faster trained networks, higher learning rates, easier to initialize weights, more activation functions viable, and simpler process of creating deep networks.

In some embodiments, a machine learning software module comprises a recurrent neural network software module. A recurrent neural network software module may be configured to receive sequential data as an input, such as consecutive data inputs, and the recurrent neural network software module updates an internal state at every time step. A recurrent neural network can use internal state (memory) to process sequences of inputs. The recurrent neural network may be applicable to tasks such as handwriting recognition or speech recognition. The recurrent neural network may also be applicable to next word prediction, music composition, image captioning, time series anomaly detection, machine translation, scene labeling, and stock market prediction. A recurrent neural network may comprise fully recurrent neural network, independently recurrent neural network, Elman networks, Jordan networks, Echo state, neural history compressor, long short-term memory, gated recurrent unit, multiple timescales model, neural Turing machines, differentiable neural computer, and neural network pushdown automata.

In some embodiments, a machine learning software module comprises a supervised or unsupervised learning method such as, for example, support vector machines (“SVMs”), random forests, clustering algorithm (or software module), gradient boosting, logistic regression, and/or decision trees. The supervised learning algorithms may be algorithms that rely on the use of a set of labeled, paired training data examples to infer the relationship between an input data and output data. The unsupervised learning algorithms may be algorithms used to draw inferences from training datasets to the output data. The unsupervised learning algorithm may comprise cluster analysis, which may be used for exploratory data analysis to find hidden patterns or groupings in process data. One example of unsupervised learning method may comprise principal component analysis. The principal component analysis may comprise reducing the dimensionality of one or more variables. The dimensionality of a given variable may be at least 1, 5, 10, 50, 100, 200, 300, 400, 500, 600, 700, 800, 900, 1,000, 1,100, 1,200 1,300, 1,400, 1,500, 1,600, 1,700, 1,800, or greater. The dimensionality of a given variables may be at most 1,800, 1,700, 1,600, 1,500, 1,400, 1,300, 1,200, 1,100, 1,000, 900, 800, 700, 600, 500, 400, 300, 200, 100, 50, 10, or less.

In some embodiments, the machine learning algorithm may comprise reinforcement learning algorithms. The reinforcement learning algorithm may be used for optimizing Markov decision processes (i.e., mathematical models used for studying a wide range of optimization problems where future behavior cannot be accurately predicted from past behavior alone, but rather also depends on random chance or probability). One example of reinforcement learning may be Q-learning. Reinforcement learning algorithms may differ from supervised learning algorithms in that correct training data input/output pairs are never presented, nor are sub-optimal actions explicitly corrected. The reinforcement learning algorithms may be implemented with a focus on real-time performance through finding a balance between exploration of possible outcomes (e.g., correct compound identification) based on updated input data and exploitation of past training.

In some embodiments, training data resides in a cloud-based database that is accessible from local and/or remote computer systems on which the machine learning-based sensor signal processing algorithms are running. The cloud-based database and associated software may be used for archiving electronic data, sharing electronic data, and analyzing electronic data. In some embodiments, training data generated locally may be uploaded to a cloud-based database, from which it may be accessed and used to train other machine learning-based detection systems at the same site or a different site.

The trained algorithm may accept a plurality of input variables and produce one or more output variables based on the plurality of input variables. The input variables may comprise one or more datasets indicative of a glycosylation feature. For example, the input variables may comprise glycoprotein sequences, data indicative of glycoprotein structure, data indicative of one or more glycosylation features, or any combination thereof.

The trained algorithm may be trained with a plurality of independent training samples. Each of the independent training samples may comprise a glycosylation feature and a sequence (e.g., a sequence or sequon comprising a glycosite, and optionally one or more structurally proximal and/or sequence proximal amino acids). The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, at least about 500, at least about 1,000, at least about 1,500, at least about 2,000, at least about 2,500, at least about 3,000, at least about 3,500, at least about 4,000, at least about 4,500, at least about 5,000, at least about, 5,500, at least about 6,000, at least about 6,500, at least about 7,000, at least about 7,500, at least about 8,000, at least about 8,500, at least about 9,000, at least about 9,500, at least about 10,000, or more independent training samples.

The trained algorithm may associate a sequence (e.g., a sequence or sequon comprising a glycosite, and optionally one or more structurally proximal and/or sequence proximal amino acids) or glycosite with a glycosylation feature at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The accuracy of associating the sequence or glycosite and the glycosylation feature by the trained algorithm may be calculated as the percentage of independent test samples (e.g., sequences or glycosites known to be associated with the glycosylation feature or known not to be associated with the glycosylation feature) that are correctly associated or not associated.

The trained algorithm may associate the sequence (e.g., a sequence or sequon comprising a glycosite, and optionally one or more structurally proximal and/or sequence proximal amino acids) or glycosite with a glycosylation feature with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of associating the sequence or glycosite and the glycosylation feature using the trained algorithm may be calculated as the percentage of sequences or glycosites classified as being associated with the glycosylation feature that truly are associated with the glycosylation feature.

The trained algorithm may associate the sequence (e.g., a sequence or sequon comprising a glycosite, and optionally one or more structurally proximal and/or sequence proximal amino acids) or glycosite with a glycosylation feature with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of associate the sequence or glycosite with a glycosylation feature using the trained algorithm may be calculated as the percentage of sequences or glycosites identified or classified as not being associated with the glycosylation feature that truly are not associated with the glycosylation feature.

The trained algorithm may associate the sequence (e.g., a sequence or sequon comprising a glycosite, and optionally one or more structurally proximal and/or sequence proximal amino acids) or glycosite with a glycosylation feature with a sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 910%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about⁹⁹0.9⁹⁹%, or more. The sensitivity of associating the sequence or glycosite with the glycosylation feature using the trained algorithm may be calculated as the percentage of independent test samples associated with the glycosylation feature (e.g., sequences or glycosites known to be associated with glycosylation feature) that are correctly identified or classified as being associated with the glycosylation feature.

The trained algorithm may be configured to associate the sequence (e.g., a sequence or sequon comprising a glycosite, and optionally one or more structurally proximal and/or sequence proximal amino acids) or glycosite with the glycosylation feature with a specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The specificity of associating the glycosylation feature using the trained algorithm may be calculated as the percentage of independent test samples associated with an absence of the glycosylation feature (e.g., sequences or glycosites known to not be associated with the glycosylation feature) that are correctly identified or classified as not associated with the glycosylation feature.

The trained algorithm may be configured to associate the sequence (e.g., a sequence or sequon comprising a glycosite, and optionally one or more structurally proximal and/or sequence proximal amino acids) or glycosite with the glycosylation feature with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying datasets derived from a sequence or glycosite as being associated or not associated with the glycosylation feature.

The trained algorithm may be adjusted or tuned to improve one or more of the performance, accuracy, PPV, NPV, sensitivity, specificity, or AUC of associating the glycosylation feature. The trained algorithm may be adjusted or tuned by adjusting parameters of the trained algorithm (e.g., a set of cutoff values used to associate a glycosylation feature as described elsewhere herein, or weights of a neural network). The trained algorithm may be adjusted or tuned continuously during the training process or after the training process has completed.

After the trained algorithm is initially trained, a subset of the inputs may be identified as most influential or most important to be included for making high-quality predictions. For example, a subset of the data may be identified as most influential or most important to be included for making high-quality associations of sequences (e.g., a sequence or sequon comprising a glycosite, and optionally one or more structurally proximal and/or sequence proximal amino acids) or glycosites and glycosylation features. The data or a subset thereof may be ranked based on classification metrics indicative of each parameter's influence or importance toward making high-quality associations of glycosylation features with sequences or glycosites. Such metrics may be used to reduce, in some embodiments significantly, the number of input variables (e.g., predictor variables) that may be used to train the trained algorithm to a desired performance level (e.g., based on a desired minimum accuracy, PPV, NPV, sensitivity, specificity. AUC, or a combination thereof). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best association metrics.

Systems and methods as described herein may use more than one trained algorithm to determine an output (e.g., association of a sequence or glycosite and glycosylation feature). Systems and methods may comprise 1, 2, 3, 4, 5, 6, 7, 8, 9, 10, 11, 12, 13, 14, 15, 16, 17, 18, 19, 20, or more trained algorithms. A trained algorithm of the plurality of trained algorithms may be trained on a particular type of data (e.g., sequence data, structural data). Alternatively, a trained algorithm may be trained on more than one type of data. The inputs of one trained algorithm may comprise the outputs of one or more other trained algorithms. Additionally, a trained algorithm may receive as its input the output of one or more trained algorithms.

### Methods and Systems for Determining Whether a Glycan Will be Found at a Glycosite

Methods and systems as described herein may determine the likelihood that one or more glycans from a plurality of candidate glycans will be found at a glycosite of a sequence (or sequon). Methods and systems may comprise providing the sequences and the plurality of candidate glycans. The methods and systems may further comprise applying a trained algorithm such as those described herein to calculate a predicted presence for one or more of the plurality of glycans at the glycosite of the sequence. The calculation may be determined based on one or more amino acids in the sequence. The methods and systems may further comprise processing (e.g., with a computer) the one or more predicted presences of the one or more glycans of the plurality of glycans, thereby determining the likelihood that the one or more glycans will be found at the glycosite of the sequence.

The predicted presence of the glycosylation feature at a glycosite in a sequence may be based on glycosylation feature structure, glycosylation feature composition, glycosylation feature length, glycosylation feature branching, sequence length, sequence composition, position of a monomer in the sequence, substitution of one or more monomers in the sequence, insertion of one or more monomers in the sequence, deletion of one or more monomers in the sequence, observed or predicted sequence secondary structure, observed or predicted sequence tertiary structure, observed or predicted sequence quaternary structure, or any combination thereof. The predicted presence may be based on a feature of the reference sequence. The predicted presence may be determined by the position of one or more amino acids in the sequence.

The likelihood of the presence or absence of the one or more glycosylation features may be determined by applying a trained algorithm as described herein to the plurality of sequences. The trained algorithm may determine a likelihood of the one or more glycosylation features being associated with a glycosite in the reference sequences. The trained algorithm may additionally determine a likelihood of the one or more glycosylation features being associated with the corresponding glycosites in the one or more variant sequences. The effect on a reference sequence may be described categorically (e.g., positive, negative, or neutral; increase or decrease), or may be described numerically (e.g., as a difference or ratio of likelihoods). In an example embodiment, the likelihood can be used to indicate predicted presence of the one or more glycosylation features. Alternatively or additionally, the likelihood of the presence or absence of the one or more glycosylation features may be determined by looking up a measure of association (e.g., logarithm of an odds ratio) between the variant sequence and the glycosylation feature and between the reference sequence and the glycosylation feature, such as in the IMR methods provided herein.

The glycosylation feature may comprise one or more monosaccharides. The glycosylation feature may comprise mannose, sialic acid, fucose, or a combination thereof. The glycosylation feature may comprise a polysaccharide epitope. The glycosylation feature may comprise a glycosylation feature listed in Table 1. In some embodiments, the glycosylation feature is an increase or decrease in a high-mannose in one of the variant sequences as compared to the reference sequence. In some embodiments, the glycosylation feature is an increase or decrease in a sialylation in one of the variant sequences as compared to the reference sequence. In some embodiments, the glycosylation feature is an increase or decrease in a high-mannose in one of the variant sequences as compared to the reference sequence. In some embodiments, the glycosylation feature is an increase or decrease in a glycosylation feature listed in Table 1.

In some embodiments, the likelihood may be expressed as a probability. In some embodiments, the likelihood may be expressed as a pseudo-probability. In some embodiments, the likelihood may be expressed as a ratio or product of one or more probabilities or pseudo-probabilities. In some embodiments, the likelihood may be expressed as a sum or difference of one or more probabilities. In some embodiments, the likelihood may be expressed as an odds ratio. In some embodiments, the likelihood may be expressed as the logarithm of an odds ratio.

### Methods and Systems for Determining the Effect of Sequence Modification on Glycosylation

Methods and systems as described herein may determine the effect of a sequence modification on glycosylation. The effect of a sequence modification may be determined by determining the likelihood (e.g., predicted presence) of one or more glycosylation features being present or absent at corresponding sites in a plurality of sequences comprising a reference sequence and one or more variant sequences. Variant sequences may differ from the reference sequence in one or more of length, monomer (e.g., amino acid, nucleotide) identity, predicted or observed secondary structure, predicted or observed tertiary structure, glycosite composition, or glycosite position. Based on the likelihood of the presence or absence of the glycosylation features in the reference sequence as compared to the variant sequences, a determination of the effect may be made.

For example, the method may comprise providing a plurality of sequences comprising reference sequence and one or more variant sequences. The one or more variant sequences may differ from the reference sequence in one or more of length, monomer (e.g., amino acid, nucleotide) identity, predicted or observed secondary structure, predicted or observed tertiary structure, glycosite composition, or glycosite position. The variant sequences may have one or more amino acid substitutions compared to the reference sequence. For each of the plurality of sequences, a trained algorithm as described herein may calculate the likelihood (e.g., predicted presence) of a glycosylation feature at the glycosite of the reference and each of the variant sequences, thereby determining the effect of the variation of the reference sequence on glycosylation of the glycosite.

The predicted presence of the glycosylation feature at a glycosite in a sequence may be based on glycosylation feature structure, glycosylation feature composition, glycosylation feature length, glycosylation feature branching, sequence length, sequence composition, position of a monomer in the sequence, substitution of one or more monomers in the sequence, insertion of one or more monomers in the sequence, deletion of one or more monomers in the sequence, observed or predicted sequence secondary structure, observed or predicted sequence tertiary structure, observed or predicted sequence quaternary structure, or any combination thereof. The predicted presence may be based on a feature of the reference sequence. Alternatively or additionally, the predicted presence may be based on a feature of one or more of the variant sequences. In some embodiments, the predicted presence is based on the identity of one or more amino acid sequences in the variant sequence(s) compared to the reference sequence. In some embodiments, the predicted presence is based on the identity of one more amino acids in the variant sequence(s) compared to the reference sequence. In some embodiments, the predicted present is based on the position of one more amino acids in the variant sequence(s) compared to the reference sequence. The position may be a distance from the glycosite in sequence or three-dimensional space.

The variant sequences may differ from the reference sequence in amino acid sequence. The difference in amino acid sequence may comprise a difference in one or more amino acid identities or positions. In some embodiments, a variant sequence of the plurality of variant sequences differs from the reference sequence in the identity of an amino acid 1, 2, 3, 4, 5, 6, 7, 8, 9, 10, 11, 12, 13, 14, 15, 16, 17, 18, 19, 20 or more positions from the glycosite. In some embodiments, the variant sequence differs from the reference sequence in the identity of an amino acid 20, 19, 18, 17, 16, 15, 14, 13, 12, 11, 10, 9, 8, 7, 6, 5, 4, 3, 2, or fewer positions from the glycosite.

The variant sequence may differ from the reference sequence in length of amino acid sequence. The variant sequence may have one or more insertions or deletions with respect to the reference sequence. The variant sequence may comprise 1, 2, 3, 4, 5, 6, 7, 8, 9, 10, 11, 12, 13, 14, 15, 16, 17, 18, 19, 20, or more insertions or deletions. The insertions or deletions may all be contiguous, or they may comprise one or more subsets across the sequence of the variant sequence. The insertion or deletion may be proximal to a glycosite of the modified glycopeptide. In some embodiments, the insertion or deletion is within 1, 2, 3, 4, 5, 6, 7, 8, 9, 10, 11, 12, 13, 14, 15, 16, 17, 18, 19, 20, or more sites of the glycosite. In some embodiments, the insertion or deletion is within no more than 20, 19, 18, 17, 16, 15, 14, 13, 12, 11, 10, 9, 8, 7, 6, 5, 4, 3, 2, or fewer sites of the glycosite. In some embodiments, the insertion or deletion is in a site distal to the glycosite. The likelihood of the presence or absence of the one or more glycosylation features may be determined by applying a trained algorithm as described herein to the plurality of sequences. The trained algorithm may determine a likelihood of the one or more glycosylation features being associated with a glycosite in the reference sequences. The trained algorithm may additionally determine a likelihood of the one or more glycosylation features being associated with the corresponding glycosites in the one or more variant sequences. The effect on a reference sequence may be described categorically (e.g., positive, negative, or neutral; increase or decrease), or may be described numerically (e.g., as a difference or ratio of likelihoods). Alternatively, the likelihood of the presence or absence of the one or more glycosylation features may be determined by looking up a measure of association (e.g., logarithm of an odds ratio) between the variant sequence and the glycosylation feature and between the reference sequence and the glycosylation feature.

The glycosylation feature may comprise one or more monosaccharides. The glycosylation feature may comprise mannose, sialic acid, fucose, high-mannose, hybrid, complexity, core or distally fucosylation, terminal sialylation, terminal galactosylation, terminal GlcNAc-ylation, GlcNAc-bisection, poly-sialylation or a combination thereof. The glycosylation feature may comprise a polysaccharide epitope. In some embodiments, the glycosylation feature may comprise one or more glycosylation features in Table 1. In some embodiments, the glycosylation feature is an increase or decrease in a high-mannose in one of the variant sequences as compared to the reference sequence. In some embodiments, the glycosylation feature is an increase or decrease in a sialylation in one of the variant sequences as compared to the reference sequence. In some embodiments, the glycosylation feature is an increase or decrease in a fucose in one of the variant sequences as compared to the reference sequence. In some embodiments, the glycosylation feature is an increase or decrease in mannose, sialic acid, fucose, high-mannose, hybrid, complexity, core or distally fucosylation, terminal sialylation, terminal galactosylation, terminal GlcNAc-ylation, GlcNAc-bisection, poly-sialylation or a combination thereof in one of the variant sequences as compared to the reference sequence. In some embodiments, the glycosylation feature is an increase or decrease in a glycosylation feature listed in Table 1.

In some embodiments, the likelihood may be expressed as a probability. In some embodiments, the likelihood may be expressed as a pseudo-probability. In some embodiments, the likelihood may be expressed as a ratio or product of one or more probabilities or pseudo-probabilities. In some embodiments, the likelihood may be expressed as a sum or difference of one or more probabilities. In some embodiments, the likelihood may be expressed as an odds ratio. In some embodiments, the likelihood may be expressed as the logarithm of an odds ratio.

In some embodiments, the method may determine that one or more sequence modifications result in an increase of the glycosylation feature. A sequence variation may be said to result in an increase of the glycosylation feature when the trained algorithm determines that the likelihood (e.g., probability, pseudo-probability) of the glycosylation feature being associated with the variant sequence is greater than the likelihood of the glycosylation feature being associated with the reference sequence. In such cases, the variant likelihood may be arbitrarily greater than the reference likelihood or the variant likelihood may have to be greater than the sequence likelihood by some cutoff value (e.g., about 1.00001, about 1.0001, about 1.001, about 1.01, about 1.1, about 1.5, about 2, about 5, about 10, or more). Alternatively or additionally, the determination of an increase of the glycosylation feature may be made by taking the ratio of the likelihoods (i.e. ratio is variant likelihood divided by reference likelihood). In such cases, the effect of the sequence variation may be to result in an increase in the glycosylation feature if the ratio of the variant likelihood to that of the reference likelihood ((variant likelihood)/(reference likelihood)) is greater than 1. The ratio may be arbitrarily greater than one or may be greater than one by some cutoff (e.g., greater than about 1.00001, about 1.0001, about 1.001, about 1.01, about 1.1, about 1.5, about 2, about 5, about 10, or more).

In some embodiments, the method may determine that one or more sequence modifications result in a decrease of the glycosylation feature. A sequence variation may be said to result in a decrease of the glycosylation feature when the trained algorithm determines that the likelihood (e.g., probability, pseudo-probability) of the glycosylation feature being associated with the variant sequence is less than the likelihood of the glycosylation feature being associated with the reference sequence. In such cases, the variant likelihood may be arbitrarily less than the reference likelihood or the variant likelihood may have to be less than the sequence likelihood by some cutoff value (e.g., 10, 1, 0.5, 0.1, 0.01, 0.001, 0.0001, 0.00001, or less). Alternatively or additionally, the determination of a decrease of the glycosylation feature may be made by taking the ratio of the likelihoods (i.e. ratio is variant likelihood divided by reference likelihood). In such cases, the effect of the sequence variation may be to result in an increase in the glycosylation feature if the ratio of the variant likelihood to that of the reference likelihood is less than 1. The ratio may be arbitrarily less than one or may be less than one by some cutoff (e.g., less than 0.9999, 0.999, 0.99, 0.9, 0.5, 0.1, 0.01, 0.001, 0.0001, 0.00001, or less).

Alternatively or additionally, the effect of a sequence modification may be determined by determining an intermolecular relation (IMR) of the glycosylation feature and the variant sequence as compared to an IMR of the glycosylation feature and the reference sequence. Based on the magnitude of the IMRs, individually or in combination, a determination of the effect may be made. For example, the method may comprise an operation of calculating the Euclidean distance of two IMRs of a one glycosylation feature between the variant sequence and the reference sequence. In some embodiments, the method comprises an operation of calculating the Euclidean distance of two IMR vectors of multiple glycosylation features between the variant sequence and the reference sequence. In some embodiments, the method comprises an operation of calculating the Euclidean distance of the identity-line-orthogonal component between two points representing four IMRs where one point represents two IMRs indicate the likelihood of one desired glycosylation feature in either the variant sequence (y-coordinate) and the reference sequence (x-coordinate) and another point represents two IMRs indicate the likelihood of one undesired and mutually exclusive to the desired glycosylation feature in either the variant sequence (y-coordinate) and the reference sequence (x-coordinate).

The IMR of a glycosylation feature and one or more sequences (e.g., variant, reference) may be determined by methods described herein. In some embodiments, the IMR may be determined by a trained algorithm. In some embodiments, the IMR may be determined by a set of generalized estimating equations. In some embodiments, the IMR may be an odds ratio. In some embodiments, the odds ratio may be determined by Fisher's exact test. In some embodiments, the odds ratio may be determined by logistic regression.

Further provided herein are method for determining the effect of a variation of a reference sequence on glycosylation of a glycosite in the reference sequence, the method comprising:
(a) providing a plurality of sequences comprising (1) the reference sequence and optionally the associated three-dimensional structure, and (2) a plurality of variant sequences, and optionally the associated three-dimensional structures, having one or more amino acid substitution as compared to the reference sequence; and (b) for each of the plurality of variant sequences, and optionally associated three-dimensional structures: applying a trained algorithm to calculate the predicted presence of a glycosylation feature at the glycosite of each variant sequence and optional associated three-dimensional structure based at least on the amino acid sequence and the optional associated three-dimensional structure of the variant sequence; thereby determining the effect of the variation of the reference sequence on glycosylation of the glycosite. In some embodiments, the glycosylation feature is a specific monosaccharide or a polysaccharide epitope. In some embodiments, the specific monosaccharide is mannose, sialic acid, fucose, D-glucose (Glc), D-galactose (Gal), N-acetylglucosamine (GlcNAc), N-acetylgalactosamine (GalNAc), D-mannose (Man), N-acetylneuraminic acid (Neu5Ac), N-glycolylneuraminic acid (Neu5Gc), neuraminic acid (Neu), 2-keto-3-deoxynononic acid or 3-deoxy-D-glycero-D-galacto-nonulosonic acid (KDN), 3-deoxy-D-manno-2 octulopyranosylonic acid (Kdo), D-galacturonic acid (GalA), L-iduronic acid (IdoA), L-rhamnose (Rha), L-fucose (Fuc), D-xylose (Xyl), D-ribose (Rib), L-arabinofuranose (Araf), D-glucuronic acid (GlcA), D-allose (All), D-apiose (Api), D-fructofuranose (Fruf), ascarylose (Asc), or ribitol (Rbo), or a combination thereof. In some embodiments, the polysaccharide epitope is high-mannose, sialylation, fucosylation, hybrid, complexity, core or distally fucosylation, terminal sialylation, terminal galactosylation, terminal GlcNAc-ylation, GlcNAc-bisection, or poly-sialylation, or a glycosylation feature listed in Table 1, or a combination thereof. In some embodiments, the glycosylation feature is an increase in high-mannose in the variant sequence as compared to the reference sequence, the glycosylation feature is decrease in high-mannose in the variant sequence as compared to the reference sequence, the glycosylation feature is an increase in sialylation in the variant sequence as compared to the reference sequence, the glycosylation feature is decrease in sialylation in the variant sequence as compared to the reference sequence, the glycosylation feature is an increase in fucosylation in the variant sequence as compared to the reference sequence, or the glycosylation feature is decrease in fucosylation in the variant sequence as compared to the reference sequence. In some embodiments, the predicted presence that the glycosite of each variant sequence will have a glycosylation feature is deternuned at least based on the identity of one or more amino acid sequences varied as compared to the reference sequence. In some embodiments, the pseudo-probability that the glycosite of each variant sequence will have a glycosylation feature is determined at least based on the position of one or more amino acid sequences varied as compared to the reference sequence. In some embodiments, the position is the distance from the glycosite. In some embodiments, each variant sequence has at least one amino acid substitution as compared to the reference sequence. In some embodiments, each variant sequence has at least two amino acid substitution as compared to the reference sequence. In some embodiments, the glycosite comprises a glycan-bound arginine, asparagine, serine, threonine, or tyrosine. In some embodiments, the glycosite further comprises one or more amino acids N-terminal to the glycan bound arginine, asparagine, serine, threonine, or tyrosine. In some embodiments, the glycosite further comprises one or more amino acids C-terminal to the glycan bound arginine, asparagine, serine, threonine, or tyrosine. In some embodiments, the sequence of a first variant sequence is comprised within a peptide. In some embodiments, the method comprises administering a therapeutically effective amount of the peptide based at least in part on determining the effect of the variation of the reference sequence on glycosylation of the glycosite.

Further provided herein are computer systems comprising a digital processing device comprising at least one processor, an operating system configured to perform executable instructions, a memory, and a computer program including instructions executable by the digital processing device to create an application for determining the effect of a variation of a reference sequence and optionally the associated three-dimensional structure on glycosylation of a glycosite in the reference sequence, the application comprising: a module programmed to, for each of a plurality of variant sequences, and optionally the associated three-dimensional structures, having one or more amino acid substitution as compared to the reference sequence, apply a trained algorithm to calculate the predicted presence of a glycosylation feature at the glycosite of each variant sequence based at least on the amino acid sequence and the optional associated three-dimensional structure of the variant sequence.

Further provided herein are non-transitory computer-readable mediums comprising machine-executable code that, upon execution by one or more computer processors, implements a method for determining the effect of a variation of a reference sequence on glycosylation of a glycosite in the reference sequence, the method comprising: (a) providing a plurality of sequon sequences comprising (1) the reference sequence and optionally the associated three-dimensional structure, and (2) a plurality of variant sequences, and optionally the associated three-dimensional structures, having one or more amino acid substitution as compared to the reference sequence; and (b) for each of the plurality of variant sequences, and optionally associated three-dimensional structures: applying a trained algorithm to calculate the predicted presence of a glycosylation feature at the glycosite of each variant sequence and optional associated three-dimensional structure based at least on the amino acid sequence and the optional associated three-dimensional structure of the variant sequence; thereby determining the effect of the variation of the reference sequence on glycosylation of the glycosite.

Further provided herein are systems for determining the effect of a variation of a reference sequence and optionally the associated three-dimensional structure on glycosylation of a glycosite in the reference sequence, the system comprising: a database comprising a plurality of sequences comprising (1) the reference sequence and optionally the associated three-dimensional structure, and (2) a plurality of variant sequences, and optionally the associated three-dimensional structures, having one or more amino acid substitution as compared to the reference sequence; and one or more computer processors operatively coupled to the database, wherein the one or more computer processors are individually or collectively programmed to: for each of the plurality of variant sequences, and optionally associated three-dimensional structures: applying a trained algorithm to calculate the predicted presence of a glycosylation feature at the glycosite of each variant sequence and optionally associated three-dimensional structure based at least on the amino acid sequence and the optional associated three-dimensional structure of the variant sequence; thereby determining the effect of the variation of the reference sequence on glycosylation of the glycosite. In some embodiments, the glycosylation feature is a specific monosaccharide or a polysaccharide epitope. In some embodiments, the specific monosaccharide is mannose, sialic acid, fucose, D-glucose (Glc), D-galactose (Gal), N-acetylglucosamine (GlcNAc), N-acetylgalactosamine (GalNAc), D-mannose (Man), N-acetylneuraminic acid (Neu5Ac), N-glycolylneuraminic acid (Neu5Gc), neuraminic acid (Neu), 2-keto-3-deoxynononic acid or 3-deoxy-D-glycero-D-galacto-nonulosonic acid (KDN), 3-deoxy-n-manno-2 octulopyranosylonic acid (Kdo), D-galacturonic acid (GalA), L-iduronic acid (IdoA), L-rhamnose (Rha), L-fucose (Fuc), D-xylose (Xyl), D-ribose (Rib), L-arabinofuranose (Araf), D-glucuronic acid (GlcA), D-allose (All), D-apiose (Api), D-fructofuranose (Fruf), ascarylose (Asc), or ribitol (Rbo), or a combination thereof. In some embodiments, the polysaccharide epitope is high-mannose, sialylation, fucosylation, hybrid, complexity, core or distally fucosylation, terminal sialylation, terminal galactosylation, terminal GlcNAc-ylation, GlcNAc-bisection, or poly-sialylation, or a glycosylation feature listed in Table 1, or a combination thereof. In some embodiments, the glycosylation feature is an increase in high-mannose in the variant sequence as compared to the reference sequence, the glycosylation feature is decrease in high-mannose in the variant sequence as compared to the reference sequence, the glycosylation feature is an increase in sialylation in the variant sequence as compared to the reference sequence, the glycosylation feature is decrease in sialylation in the variant sequence as compared to the reference sequence, the glycosylation feature is an increase in fucosylation in the variant sequence as compared to the reference sequence, or the glycosylation feature is decrease in fucosylation in the variant sequence as compared to the reference sequence. In some embodiments, the pseudo-probability that the glycosite of each variant sequence will have a glycosylation feature is determined at least based on the identity of one or more amino acid sequences varied as compared to the reference sequence. In some embodiments, the pseudo-probability that the glycosite of each variant sequence will have a glycosylation feature is determined at least based on the position of one or more amino acid sequences varied as compared to the reference sequence. In some embodiments, the position is the distance from the glycosite. In some embodiments, each variant sequence has one amino acid substitution as compared to the reference sequence. In some embodiments, each variant sequence has at least two amino acid substitution as compared to the reference sequence. In some embodiments, the glycosite comprises an arginine, asparagine, serine, threonine, or tyrosine. In some embodiments, the glycosite further comprises one or more amino acids N-terminal to the glycan bound arginine, asparagine, serine, threonine, or tyrosine. In some embodiments, the glycosite further comprises one or more amino acids C-terminal to the glycan bound arginine, asparagine, serine, threonine, or tyrosine. In some embodiments, the sequence of a variant sequence is comprised within a peptide. Further provided are methods of treatment comprising administering to a subject in need thereof a therapeutically effective amount of the peptide.

Further provided herein are methods of modifying a reference glycopeptide to alter a glycosylation feature of a glycosite of the reference glycopeptide to produce a modified glycopeptide, the method comprising: identifying a predicted presence of the glycosylation feature at a glycosite of a modified glycopeptide, which modified glycopeptide comprises one or more amino acid substitutions to a sequence of the reference glycopeptide, and generating the modified glycopeptide having the one or more amino acid substitutions in the sequence of the reference glycopeptide if the predicted presence is at least a threshold predicted presence. In some embodiments, the threshold pseudo-probability is about 50%, 60%, 70%, 80%, 90%, or higher. In some embodiments, the predicted presence is determined using a trained algorithm. In some embodiments, the predicted presence is determined at least based on the identity of one or more amino acids varied as compared to the reference sequence. In some embodiments, the predicted presence is determined at least based on the position of one or more amino acids varied as compared to the reference sequence. In some embodiments, the position is the distance from the glycosite.

Further provided herein are methods of modifying a reference glycopeptide to alter a glycosylation feature of a glycosite of the reference glycopeptide to produce a modified glycopeptide, the method comprising: substituting one or more amino acids within 15 amino acids of the glycosite to generate the modified glycopeptide. In some embodiments, the glycosylation feature is high-mannose, sialylation, fucosylation, or a combination thereof, the glycosylation feature is an increase in high-mannose in the modified glycopeptide as compared to the reference glycopeptide, the glycosylation feature is decrease in high-mannose in the modified glycopeptide as compared to the reference glycopeptide, the glycosylation feature is an increase in sialylation in the modified glycopeptide as compared to the reference glycopeptide, the glycosylation feature is decrease in sialylation in the modified glycopeptide as compared to the reference glycopeptide, the glycosylation feature is an increase in fucosylation in the modified glycopeptide as compared to the reference glycopeptide, or the glycosylation feature is decrease in fucosylation in the modified glycopeptide as compared to the reference glycopeptide. In some embodiments, the glycosite comprises an arginine, asparagine, serine, threonine, or tyrosine. In some embodiments, the method further comprises administering a therapeutically effective amount of the modified glycopeptide to a subject in need thereof based at least in part on the altered glycosylation feature of the modified glycopeptide.

Further provided herein are modified glycopeptides having a first glycosylation feature that is different from a reference glycosylation feature of a glycosite of a reference glycoprotein, wherein the modified glycopeptide has one or more amino acid substitutions in a sequence comprising the glycosite as compared to the reference glycoprotein. In some embodiments, the one or more amino acid substitutions is positioned within 1, 2, 3, 4, 5, 6, 7, 8, 9, 10, 11, 12, 13, 14, 15, 16, 17, 18, 19, 20, 21, 22, 23, 24, or 25 amino acids of the glycosite; or wherein the one or more amino acid substitutions is positioned within a sequon comprising the glycosite. In some embodiments, the first glycosylation feature is a specific monosaccharide or a polysaccharide epitope. In some embodiments, the specific monosaccharide is mannose, sialic acid, fucose, D-glucose (Glc), D-galactose (Gal), N-acetylglucosamine (GlcNAc), N-acetylgalactosamine (GalNAc), D-mannose (Man), N-acetylneuraminic acid (Neu5Ac), N-glycolylneuraminic acid (Neu5Gc), neuraminic acid (Neu), 2-keto-3-deoxynononic acid or 3-deoxy-D-glycero-D-galacto-nonulosonic acid (KDN), 3-deoxy-D-manno-2 octulopyranosylonic acid (Kdo), D-galacturonic acid (GalA), L-iduronic acid (IdoA), L-rhamnose (Rha), L-fucose (Fuc), D-xylose (Xyl), D-ribose (Rib), L-arabinofuranose (Araf), n-glucuronic acid (GlcA), D-allose (All), D-apiose (Api), D-fructofuranose (Fruf), ascarylose (Asc), or ribitol (Rbo), or a combination thereof. In some embodiments, the polysaccharide epitope is high-mannose, sialylation, fucosylation, hybrid, complexity, core or distally fucosylation, terminal sialylation, terminal galactosylation, terminal GlcNAc-ylation, GlcNAc-bisection, or poly-sialylation, or a glycosylation feature listed in Table 1, or a combination thereof. In some embodiments, the first glycosylation feature is an increase in high-mannose in the modified glycopeptide as compared to the reference glycopeptide, the first glycosylation feature is decrease in high-mannose in the modified glycopeptide as compared to the reference glycopeptide, the first glycosylation feature is an increase in sialylation in the modified glycopeptide as compared to the reference glycopeptide, the first glycosylation feature is decrease in sialylation in the modified glycopeptide as compared to the reference glycopeptide, the first glycosylation feature is an increase in fucosylation in the modified glycopeptide as compared to the reference glycopeptide, or the first glycosylation feature is decrease in fucosylation in the modified glycopeptide as compared to the reference glycopeptide. In some embodiments, the glycosite comprises an arginine, asparagine, serine, threonine, or tyrosine. Further provided is a method comprising administering a therapeutically effective amount of the modified glycopeptide to a subject in need thereof based at least in part on the first glycosylation feature of the modified glycopeptide.

Further provided herein are methods for determining the likelihood that one or more glycans from a plurality of candidate glycans will be found at a glycosite of a sequence, the method comprising: (a) providing the sequence (and optionally the associated three-dimensional structure) and the plurality of candidate glycans; (b) for each of the plurality of candidate glycans: applying a trained algorithm to calculate a predicted presence for each glycan at the glycosite of the sequence determined at least based on one or more amino acids in the sequence (and optionally the associated three-dimensional structure); and (c) computer processing the predicted presence for each of the plurality of candidate glycans to determine the likelihood that the one or more glycans will be found at the glycosite of the sequence. In some embodiments, the one or more glycans comprises at least one glycan of Table 1. In some embodiments, the predicted presence of the glycan at the glycosite is determined at least based on the identity of the one or more amino acids in the sequence. In some embodiments, the predicted presence of the glycan at the glycosite is determined at least based on the position of the one or more amino acids in the sequence. In some embodiments, the predicted presence of the glycan at the glycosite is determined at least based on the identity and position of the one or more amino acids in the sequence. In some embodiments, the one or more amino acids in the sequence is located within 1, 2, 3, 4, 5, 6, 7, 8, 9, or 10 amino acids of the glycosite. In some embodiments, the glycosite comprises an arginine, asparagine, serine, threonine, or tyrosine. In some embodiments, the glycosite further comprises one or more amino acids N-terminal to the glycan bound arginine, asparagine, serine, threonine, or tyrosine. In some embodiments, the glycosite further comprises one or more amino acids C-terminal to the glycan bound arginine, asparagine, serine, threonine, or tyrosine. In some embodiments, the sequence is comprised within a peptide. In some embodiments, precursors of the one or more glycans are glycans present in a host cell during production of the peptide. In some embodiments, precursors of the one or more glycans are glycans present in a host cell medium during production of the peptide. In some embodiments, the method further comprises administering a therapeutically effective amount of the peptide based at least in part on determining whether the one or more glycans will be found at the glycosite of the sequence.

Further provided herein are computer systems comprising a digital processing device comprising at least one processor, an operating system configured to perform executable instructions, a memory, and a computer program including instructions executable by the digital processing device to create an application for determining the likelihood that one or more glycans from a plurality of candidate gly cans will be found at a glycosite of a sequence, the application comprising: (a) a module programmed to, for each of the plurality of candidate glycans, apply a trained algorithm to calculate a predicted presence for each glycan at the glycosite of the sequence determined at least based on one or more amino acids in the sequence (and optionally the associated three-dimensional structure of the sequence) to generate a plurality of predicted presences; and (b) a processing module programmed to process the plurality of predicted presences to determine the likelihood that the one or more glycans will be found at the glycosite of the sequence.

Further provided herein are non-transitory computer-readable mediums comprising machine-executable code that, upon execution by one or more computer processors, implements a method for determining the likelihood that one or more glycans from a plurality of candidate glycans will be found at a glycosite of a sequence, the method comprising: (a) providing the sequence (and optionally the associated three-dimensional structure) and the plurality of candidate glycans; (b) for each of the plurality of candidate glycans: applying a trained algorithm to calculate a predicted presence for each glycan at the glycosite of the sequence determined at least based on one or more amino acids in the sequence (and optionally the associated three-dimensional structure); and (c) computer processing the predicted presence for each of the plurality of candidate glycans to determine the likelihood that the one or more glycans will be found at the glycosite of the sequence.

Further provided herein are systems for determining the likelihood that one or more glycans from a plurality of candidate glycans will be found at a glycosite of a sequence, the system comprising: a database comprising the plurality of candidate glycans; and one or more computer processors operatively coupled to the database, wherein the one or more computer processors are individually or collectively programmed to: (a) for each of the plurality of candidate glycans: apply a trained algorithm to calculate a predicted presence for each glycan at the glycosite of the sequence determined at least based on one or more amino acids in the sequence (and optionally the associated three-dimensional structure of the sequence); and (c) process the predicted presence for each of the plurality of candidate glycans to determine the likelihood that the one or more glycans will be found at the glycosite of the sequence. In some embodiments, the one or more glycans comprises at least one glycan of Table 1. In some embodiments, the predicted presence for the glycan at the glycosite of the sequence is determined at least based on the identity of the one or more amino acids in the sequence. In some embodiments, the predicted presence for the glycan at the glycosite of the sequence is determined at least based on the position of the one or more amino acids in the sequence. In some embodiments, the predicted presence for the glycan at the glycosite of sequence is determined at least based on the identity and position of the one or more amino acids in the sequence. In some embodiments, the one or more amino acids in the sequence is located within 1, 2, 3, 4, 5, 6, 7, 8, 9, or 10 amino acids of the glycosite. In some embodiments, the glycosite comprises an arginine, asparagine, serine, threonine, or tyrosine. In some embodiments, the glycosite further comprises one or more amino acids N-terminal to the glycan bound arginine, asparagine, serine, threonine, or tyrosine. In some embodiments, the glycosite further comprises one or more amino acids C-terminal to the glycan bound arginine, asparagine, serine, threonine, or tyrosine. In some embodiments, the sequence is comprised within a peptide. In some embodiments, precursors of the one or more glycans are glycans present in a host cell during production of the peptide. In some embodiments, precursors of the one or more glycans are glycans present in a host cell medium during production of the peptide.

Further provided herein are methods of modifying a reference glycopeptide to alter a glycan substructure of a glycosite of the reference glycopeptide to produce a modified glycopeptide, the method comprising: calculating whether there is a positive or negative IMR association between one or more amino acid substitutions of a protein feature proximal to the glycosite and the glycan substructure, and generating the modified glycopeptide having the one or more amino acid substitutions if a magnitude of the IMR association is at least a threshold value. In some embodiments, the threshold value is about 50%, 60%, 70%, 80%, 90%, or higher. In some embodiments, the IMR is as generalized estimating equation (GEE) IMR. In some embodiments, the IMR is a Fisher's exact test IMR. In some embodiments, the IMR is significant if it has a false discovery rate (FDR) correction less than about 0.1. In some embodiments, the IMR is significant if it has a p-value less than about 0.05. In some embodiments, the IMR comprises a logarithm of an odds ratio (log OR) with a magnitude greater then about 1. In some embodiments, the IMR comprises a log OR with a magnitude greater then about 0.5. In some embodiments, the IMR comprises a log OR with a magnitude greater then about 0.1. In some embodiments, the IMR association is determined using a matrix describing the expected glycoimpact of the one or more amino acid substitutions. In some embodiments, the IMR association is determined at least based on the identity of one or more amino acids. In some embodiments, the IMR association is determined at least based on the proximity of the one or more amino acids to the glycosite. In some embodiments, the proximity is the distance from the glycosite as measured in angstroms. In some embodiments, the proximity is less than or equal to about 6 angstroms to about 25 angstroms. In some embodiments, the proximity is the number of amino acids between the each of the one or more amino acids and the glycosite. In some embodiments, the distance is about 1, 2, 3, 4, 5, 6, 7, 8, 9, 10, 11, 12, 13, 14, or 15 amino acids.

Further provided are methods of modifying a reference glycopeptide to alter a glycan substructure of a glycosite of the reference glycopeptide to produce a modified glycopeptide, the method comprising: substituting one or more amino acids of a protein feature proximal to the glycosite to generate the modified glycopeptide. In some embodiments, the protein feature proximal to the glycosite comprises a structural feature. In some embodiments, the structural feature is less than or equal to about 6 angstroms to about 25 angstroms from the glycosite. In some embodiments, the structural feature is a secondary structure comprising a beta strand, alpha helix, extended strand, beta-bridge, turn, or bend, or a combination of two or more thereof. In some embodiments, the protein feature proximal to the glycosite comprises an amino acid within about 6 amino acids of the glycosite in the N- or C-terminal direction. In some embodiments, the glycan substructure is selected from Table 2 or Table 3. In some embodiments, the method employs a computational approach. In some embodiments, the structure of the reference glycopeptide is or has been determined using X-ray crystallography, homology modeling, and/or de novo prediction based on primary amino acid sequence. In some embodiments, the method further comprises administering a therapeutically effective amount of the modified glycopeptide to a subject in need thereof based at least in part on the altered glycan substructure of the modified glycopeptide.

Further provided are modified glycopeptides having a first glycan substructure that is different from a reference glycan substructure of a glycosite of a reference glycoprotein, wherein the modified glycopeptide has one or more amino acid substitutions of a protein feature proximal to the glycosite as compared to the reference glycoprotein. In some embodiments, the protein feature proximal to the glycosite comprises a structural feature. In some embodiments, the structural feature is less than or equal to about 6 angstroms to about angstroms from the glycosite. In some embodiments, the structural feature is a secondary structure comprising a beta strand, alpha helix, extended strand, beta-bridge, turn, or bend, or a combination of two or more thereof. In some embodiments, the protein feature proximal to the glycosite comprises an amino acid within about 6 amino acids of the glycosite in the N- or C-terminal direction. In some embodiments, the glycan substructure is selected from Table 2 or Table 3. In some embodiments, the protein feature is selected from Table 2 or Table 3.

Further provided are methods comprising administering a therapeutically effective amount of the modified glycopeptide of any one of claims 117-123 to a subject in need thereof based at least in part on the first glycan substructure of the modified glycopeptide.

Further provided are modified glycopeptides having an increase, decrease, or change in a glycan structure at a glycosite of the modified glycopeptide as compared to a reference glycopeptide, as determined based on the associations of Table 2 and/or Table 3 (e.g., wherein the modified glycoprotein has a Phe within 5 amino acids upstream of the glycosite, and the reference glycopeptide does not have a Phe within 5 amino acids upstream of the glycosite of the reference glycopeptide).

Further provided are methods comprising administering a therapeutically effective amount of the modified glycopeptide of claim 125 to a subject in need thereof based at least in part on the increase, decrease, or change any of the glycan features selected from Table 1.

Further provided are methods for determining the effect of a variation of a reference sequence on glycosylation of a first glycosite in the reference sequence, wherein the reference sequence comprises the first glycosite and a second glycosite, the method comprising: (a) providing a plurality of sequences comprising (1) the reference sequence and (2) a plurality of variant sequences each having a different glycosylation feature at the second glycosite as compared to the reference sequence; and (b) for each of the plurality of variant sequences: applying a trained algorithm to calculate the predicted presence of a glycosylation feature at the first glycosite based at least on the identity of the glycosylation feature at the second glycosite; thereby determining the effect of the variation of the reference sequence on glycosylation of the first glycosite.

Further provided are methods for determining the effect of a variation of the structure of a reference sequence on glycosylation of a glycosite in the reference sequence, the method comprising: (a) providing a plurality of sequences comprising (1) the reference sequence and (2) a plurality of variant sequences having one or more amino acid substitution as compared to the reference sequence; and (b) for each of the plurality of variant sequences: applying a trained algorithm to calculate the predicted presence of a glycosylation feature at the glycosite of each variant sequence based at least on the structure of the variant sequence; thereby determining the effect of the variation of the reference sequence structure on glycosylation of the glycosite. In some embodiments, the structure is secondary structure, tertiary structure, or quaternary structure, or a combination of two or more thereof.

In any of the methods and systems herein, the sequence may be a viral sequence. For instance, in some embodiments, provided herein are methods for determining the likelihood that one or more glycans from a plurality of candidate glycans will be found at a glycosite of a viral sequence, the method comprising: (a) providing the viral sequence and the plurality of candidate glycans, observed glycans, desired glycans, undesired glycans; (b) for each of the plurality of candidate, observed, desired, or undesired glycans at each glycosite: applying a trained algorithm to calculate a predicted presence for each glycan at the glycosite of the sequence; and (c) computer processing the predicted presence for each of the plurality of candidate, observed, desired, or undesired glycans to determine the likelihood that the one or more glycans will be found at the glycosite of the sequence.

In any of the methods and systems herein, the likelihood of a disease or disorder associated with a glycoprotein in an individual may be determined. For instance, in some embodiments, provided herein is are methods of determining a likelihood of a disease or disorder associated with a glycoprotein in an individual, the method comprising: calculating a first IMR association between a glycosite of the glycoprotein and a glycosylation feature; calculating a second IMR association between said glycosylation feature and a glycosite of a modified glycoprotein, wherein said modified glycoprotein comprises one or more amino acid substitutions relative to the glycoprotein; and determining said likelihood based on the difference of said first IMR and said second IMR.

In some embodiments, methods and systems include and/or utilize: (1) a trained algorithm applied to calculate a predicted presence for each glycan, a plurality of glycans, a plurality of glycans containing a glycan feature, or a plurality of glycans lacking a glycan feature at the glycosite of the sequence, (2) IMRs to estimate the likelihood of a glycan feature existing at the glycosite of a sequence, and/or (3) predicted presence from a trained algorithm or IMRs are compared between a multiplicity of sequences (e.g. reference sequence and variant sequence) to determine the relative likelihood of a glycan feature being present.

In some embodiments, methods are provided for determining the importance of a glycosite by examining co-evolution (e.g. evolutionary coupling) or conservation (e.g. glycan or composition associated conservation around a glycosite) with defining glycosite features (e.g. N, T, or S where the glycan attaches, the T or S at position N+2). A method of determining the importance of a glycosite by identifying elevated (relative to baseline evolutionary coupling or conservation with any amino acid, any asparagine, any serine, or any threonine) evolutionary coupling with a defining glycosite feature (e.g. N, T, or S where the glycan attaches, the T or S at position N+2).

Any of the methods and systems herein may be used to: (1) determine the likelihood a glycan feature can be added to a glycosite, (2) determine the likelihood a glycan feature can be enriched at a glycosite, (3) determine the likelihood a glycan feature can be removed from a glycosite, (4) determine the likelihood a glycan feature can be depleted at a glycosite, (5) aid in synthetic methods of glycan biosynthesis, (6) determine the change in glycoylstion due to mutation as it may relate to pathogenicity, (7) determine the change in glycosylation due to sequence differences between a reference and mutant sequence, (8) to explain the biological methods underlying glycan-modulated pathologies, (9) add, remove, enrich, deplete a glycan or glycan feature for the purposes of genetic therapy wherein the modified glycoconjugate is pathogenic, corrective of a pathogenic mechanism, or competitively inhibitory of a pathogenic mechanism, (10) predict glycosylation on any glycoconjugate including proteins, peptides, sequences, nucleotides, lipids, sugars, phosphates, functional groups, or small molecules, (11) glycoengineer surface proteins or targeting receptors for the purpose of developing cell therapies (e.g. CAR-T cells), (12) change the behavior, functional impact, biological importance, biological mechanism of a glycoconjugate without changing the structure, or (13) any combination of two or more thereof.

### Methods and Systems for Modifying Glycopeptides

Methods and systems as described herein may modify a sequence or sequon comprising a glycosite, (in some cases referred to as a glycopeptide). A glycopeptide may be modified by altering the sequence or amino acid composition of the peptide (e.g., by modifying an amino acid sequence proximal to a glycosite). A glycosylation feature maybe be modified by altering an amino acid close to the glycosite (e.g., structurally proximal to the glycosite). Methods for modifying glycopeptides may comprise identifying the likelihood that one or more changes to the reference glycopeptide will alter a glycosylation feature of the glycosite to give a modified glycopeptide. The likelihood may be determined by applying a trained algorithm as described herein to the reference glycopeptide. Alternatively or additionally, the likelihood may be determined by calculating or looking up an association score between the reference glycopeptide and the glycosylation feature. The association score may be an IMR or combination of IMRs as described herein.

Changes to the glycopeptide to give a modified glycopeptide may comprise one or more of length, monomer (e.g., amino acid, nucleotide) identity, predicted or observed secondary structure, predicted or observed tertiary structure, glycosite composition, or glycosite position. Based on the likelihood of the presence or absence of the glycosylation features in the original glycopeptide as compared to the modified glycopeptide, a determination of the effect may be made.

In some embodiments, the likelihood may be expressed as a probability. In some embodiments, the likelihood may be expressed as a pseudo-probability. In some embodiments, the likelihood may be expressed as a ratio or product of one or more probabilities or pseudo-probabilities. In some embodiments, the likelihood may be expressed as a product, quotient, sum or difference of one or more probabilities. In some embodiments, the likelihood may be expressed as an odds ratio. In some embodiments, the likelihood may be expressed as the logarithm of an odds ratio. In some embodiments, the likelihood may be expressed as a mathematical operation performed on one or more odds, odds ratios, log odds ratios, probabilities or pseudo-probabilities.

In one example embodiments, the predicted presence (specific instance of likelihood) of the glycosylation feature at a glycosite in a sequence may be based on glycosylation feature structure, glycosylation feature composition, glycosylation feature length, glycosylation feature branching, sequence length, sequence composition, position of a monomer in the sequence, substitution of one or more monomers in the sequence, insertion of one or more monomers in the sequence, deletion of one or more monomers in the sequence, observed or predicted sequence secondary structure, observed or predicted sequence tertiary structure, observed or predicted sequence quaternary structure, or any combination thereof. The predicted presence may be based on a feature of the reference sequence. Alternatively or additionally, the predicted presence may be based on a feature of one or more of the variant sequences.

The method may further comprise determining that the likelihood is above or below a cutoff or threshold value. Examples of threshold values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

In some embodiments, the likelihood is expressed as the logarithm of an odds ratio. The odds ratio may be determined from Fisher's exact test. The odds ratio may be determined by solving a set of generalized estimating equations. In some embodiments, the association score may be an IMR as described herein. In some embodiments, the IMR may be a generalized estimating equation (GEE) parameter. In some embodiments, the IMR may be an odds ratio as determined by Fisher's exact test.

The modified glycopeptide may differ from the reference glycopeptide in amino acid sequence. The difference in amino acid sequence may comprise a difference in one or more amino acid identities or positions. In some embodiments, the variant glycopeptide differs from the reference glycopeptide in the identity of an amino acid 1, 2, 3, 4, 5, 6, 7, 8, 9, 10, 11, 12, 13, 14, 15, 16, 17, 18, 19, 20 or more positions from the glycosite. In some embodiments, the variant glycopeptide differs from the reference glycopeptide in the identity of an amino acid 20, 19, 18, 17, 16, 15, 14, 13, 12, 11, 10, 9, 8, 7, 6, 5, 4, 3, 2, or fewer positions from the glycosite.

The modified glycopeptide may differ from the reference glycopeptide in length of amino acid sequence. The modified glycopeptide may have one or more insertions or deletions with respect to the reference glycopeptide. The modified glycopeptide may comprise 1, 2, 3, 4, 5, 6, 7, 8, 9, 10, 11, 12, 13, 14, 15, 16, 17, 18, 19, 20, or more insertions or deletions. The insertions or deletions may all be contiguous, or they may comprise one or more subsets across the sequence of the modified glycopeptide. The insertion or deletion may be proximal to a glycosite of the modified glycopeptide. In some embodiments, the insertion or deletion is within 1, 2, 3, 4, 5, 6, 7, 8, 9, 10, 11, 12, 13, 14, 15, 16, 17, 18, 19, 20, or more sites of the glycosite. In some embodiments, the insertion or deletion is within no more than 20, 19, 18, 17, 16, 15, 14, 13, 12, 11, 10, 9, 8, 7, 6, 5, 4, 3, 2, or fewer sites of the glycosite. In some embodiments, the insertion or deletion is in a site distal to the glycosite.

The modified glycopeptide may differ from the reference glycopeptide in one or more glycosylation features. The modified glycopeptide may differ from the reference glycopeptide in 1, 2, 3, 4, 5, 6, 7, 8, 9, 10, 20, 30, 40, 50, 60, 70, 80, 90, 100, or more glycosylation features. The one or more glycosylation features may be identical, or they may be independently distinct.

In some embodiments, the glycopeptide is a naturally occurring glycoprotein or derivative or modification thereof. In some embodiments, the glycopeptide comprises an antibody, glycoconjugate, Fc fusion protein, anticoagulant, blood factor, bone morphogenetic protein, engineered protein scaffold, enzyme, growth factor, hormone, interferon, interleukin or other cytokine, viral (or other pathogen) protein (or antigens), part of proteins (truncated proteins, ectodomains, stem domains, etc.), chimeric protein (e.g., chimeric antigen receptor), or thrombolytic. In some embodiments, the glycopeptide may be used in glycopeptide-based therapies.

In some embodiments, the modified glycopeptide is a modified form of an antigen or other molecule derived form a pathogen. In some embodiments, the pathogen is selected from the group consisting of a virus, bacterium, prion, fungus, protozoon, viroid, and parasite.

In some embodiments, the pathogen is selected from the group that causes human disease which includes but are not limited to, Bacillus anthracis (anthrax), Clostridium botulinum toxin (botulism), Yersinia pestis (plague). Variola major (smallpox) and other related pox viruses, Francisella tularensis (tularemia), Viral hemorrhagic fevers, Arenaviruses, (e.g., Junin, Machupo, Guanarito, Chapare, Lassa, and/or Lujo), Bunyaviruses (e.g., Hantaviruses causing Hanta Pulmonary syndrome, Rift Valley Fever, and/or Crimean Congo Hemorrhagic Fever), Flaviviruses, Dengue, Filoviruses (e.g., Ebola and Marburg viruses), Burkholderia pseudomallei (melioidosis), Coxiella bumetii (Q fever), Brucella species (brucellosis), Burkholderia mallei (glanders), Chlamydia psittaci (Psittacosis), Ricin toxin (Ricinus communis), Epsilon toxin (Clostridium perfringens), Staphylococcus enterotoxin B (SEB), Typhus fever (Rickettsia prowazekii), Food and water-borne pathogens, Diarrheagenic E. coli, Pathogenic Vibrios, Shigella species, Salmonella, Listeria monocytogenes, Campylobacter jejuni, Yersinia enterocolitica, Caliciviruses, Hepatitis A, Cryptosporidium parvuni, Cyclospora cayatanensis, Giardia lamblia, Entamoeba histolytica, Toxoplasma gondii, Naegleria fowleri, Balamuthia mandrillaris, Fungi, Microsporidia, Mosquito-borne viruses (e.g., West Nile virus (WNV), LaCrosse encephalitis (LACV), California encephalitis, Venezuelan equine encephalitis (VEE), Eastern equine encephalitis (EEE), Western equine encephalitis (WEE), Japanese encephalitis virus (JE), St. Louis encephalitis virus (SLEV), Yellow fever virus (YFV), Chikungunya virus, Zika virus, Nipah and Hendra viruses, Additional hantaviruses, Tickborne hemorrhagic fever viruses, Bunyaviruses, Severe Fever with Thrombocytopenia Syndrome virus (SFTSV), Heartland virus, Flaviviruses (e.g., Omsk Hemorrhagic Fever virus, Alkhurma virus, Kyasanur Forest virus), Tickbome encephalitis complex flaviviruses. Tickborne encephalitis viruses, Powassan/Deer Tick virus, Tuberculosis, including drug-resistant Tuberculosis, Influenza virus, Prions, Streptococcus, Pseudomonas, Shigella, Campylobacter, Salmonella, Clostridium, Escherichia, Hepatitis C, papillomavirus, Epstein-Barr virus, varicella, variola, Orthomyxovirus, Severe acute respiratory syndrome associated coronavirus (SARS-CoV), SARS-CoV-2 (COVID-19), MERS-CoV, other highly pathogenic human coronaviruses, or any combination thereof.

In some embodiments, the virus is a respiratory virus that primarily results in respiratory symptoms including, without limitation, coronaviruses, influenza viruses, adenoviruses, rhinoviruses, coxsackieviruses, and metapneumoviruseses. In some embodiments, the virus is an enteric virus that primarily results in digestive symptoms including, without limitation, enteroviruses, noroviruses, heptoviruses, reoviruses, rotaviruses, parvoviruses, toroviruses, and mastadenovirus. In certain embodiments, the virus is a hemorrhagic fever virus including, without limitation, Ebola virus, Marburg virus, dengue fever virus, yellow fever virus, Rift valley fever virus, hanta virus, and Lassa fever virus.

In some embodiments, the pathogen-associated antigen is from an influenza virus. In some embodiments, the pathogen-associated antigen is from an influenza A virus, such as the H5N1 strain. In some embodiments, the pathogen-associated antigen is from an influenza B virus. In some embodiments, the pathogen-associated antigen is an influenza matrix M1 protein or a fragment thereof. In some embodiments, the pathogen-associated antigen is an influenza neuraminidase or a fragment thereof. In some embodiments, the pathogen-associated antigen is an influenza hemagglutinin or a fragment thereof. For example, the pathogen-associated antigen may comprise an entire hemagglutinin, an HA1 domain, an HA2 domain or any antigenic portion thereof.

In some embodiments, the pathogen-associated antigen is a Coronaviridae antigen. In some embodiments, the Coronaviridae exhibits human tropism. In some cases, the Coronaviridae is selected from the list consisting of SARS Coronavirus (SARS-CoV-1), COVID-19 (SARS-CoV-2), MERS-coronavirus (MERS-CoV), or any combination thereof. In some embodiments, the Coronaviridae comprises SARS Coronavirus (SARS-CoV-1). In some embodiments, the Coronaviridae comprises COVID-19 (SARS-CoV-2). In some embodiments, the Coronaviridae comprises MERS-coronavirus (MERS-CoV). In some embodiments, the Coronaviridae antigen comprises a spike protein, an envelope protein, a nucleocapsid protein, a membrane protein, a membrane glycoprotein, or a non-structural protein. In some embodiments, the Coronaviridae antigen comprises a spike protein, an envelope small membrane protein, a membrane protein, a non-structural protein 6 (NSP6), a nucleoprotein, an ORF10 protein, Protein 3a, Protein7a, Protein 9b, structural protein 8, uncharacterized protein 4, or any combination thereof.

The method may comprise an operation of generating the glycopeptide. The glycopeptide may be generated by any suitable biochemical process (e.g., expression in a natural or recombinant host organism or part thereof), chemical synthetic route (e.g., solid-phase glycan synthesis), or combination thereof. In some embodiments, the glycopeptide may be generated if the likelihood is determined to be above a cutoff value. Alternatively, the glycopeptide may be generated if the likelihood is determined to be below a cutoff value.

In some embodiments, a glycopeptide may be generated by culturing cells in vivo. A cell may comprise a cell membrane, at least one chromosome, composed of genetic material, cytoplasm, and various organelles which are adapted or specialized to perform one or more vital functions, such as energy and proteins synthesis, respiration, digestion, storage and transportation of nutrients, locomotion, or cell division. A cell may comprise one or a plurality of cells. A cell may comprise a somatic cell, a terminally differentiated cell, a stem cell, or a germ cell. A somatic cell may be any cell forming the body of an organism that are not germline cells. Mutations in somatic cells may affect the individual organism but are not passed onto offspring. A terminally differentiated cell may refer to any cell that in the course of acquiring specialized functions, is not able to transform into other types of cells. These cells may constitute most of the mammalian body and may be unable to proliferate.

In some embodiments, a glycoprotein may be generated by culturing cells in vitro. A cell may comprise a cell membrane, at least one chromosome, composed of genetic material, cytoplasm, and various organelles which are adapted or specialized to perform one or more vital functions, such as energy and proteins synthesis, respiration, digestion, storage and transportation of nutrients, locomotion, or cell division. A cell may comprise one or a plurality of cells. A cell may comprise a somatic cell, a terminally differentiated cell, a stem cell, a germ cell, or other cell type. A somatic cell may be any cell forming the body of an organism that are not germline cells. Mutations in somatic cells may affect the individual organism but are not passed onto offspring.

In some embodiments, a glycoprotein may be generated by a cell-free synthetic process. The cell-free synthetic process may use the constituent biomolecules, enzymes, substrates, cofactors, or reagents of an organism, recombinant or otherwise modified constituent biomolecules, enzymes, substrates, cofactors, or reagents of an organism; or catalysts, reagents, and reaction conditions not associated with biological systems, such as those which may be employed in a chemical laboratory setting.

In some embodiments, the modified glycoprotein may be used as part of a pharmaceutical composition as described elsewhere herein. In some embodiments, the modified glycoprotein may be used in a vaccine composition. In some embodiments, the vaccine is a viral vaccine.

Methods and systems as described herein are not limited to modification of glycopeptides. Other biomolecules associated with glycans or glycosylation features may be modified by the methods described herein. In some embodiments, the method may be used to produce a modified glycosylated nucleic acid. In some embodiments, the method may be used to produce a modified glycosylated lipid.

### Methods and Systems for Predicting Pathogenicity of a Mutation

Methods and systems as described herein may predict whether a mutation in a glycoprotein is pathogenic. In some cases, the methods and systems may determine the likelihood that a subject (e.g., an individual) has or is predicted to have a disease or disorder associated with a glycoprotein. Methods for determining the likelihood of the individual having the disease or disorder associated with the glycoprotein may comprise calculating a first IMR between a glycosite of the glycoprotein and a glycosylation feature. The methods may further comprise an operation of determining a second IMR based on the glycosylation feature and the glycosite in a modified glycoprotein. The modified glycoprotein may differ from the first glycoprotein in one or more amino acids (e.g., substitutions, deletions, etc.). In some cases, the modified glycoprotein corresponds to a mutant or variant glycoprotein associated with a known disease or disorder. Based on the first IMR and the second IMR, the likelihood of the individual having the disease or disorder may be determined. In some embodiments, any methods and systems provided herein for determining the effect of sequence modification on glycosylation may be used to identify changes in glycosylation. In some embodiments, any methods and systems for modifying glycopeptides provided herein may be used to identify changes in glycosylation.

Changes to the glycoprotein relative to the modified glycoprotein may comprise one or more of length, monomer (e.g., amino acid) identity, predicted or observed secondary structure, predicted or observed tertiary structure, glycosite composition, or glycosite position. Based on the likelihood of the presence or absence of the glycosylation features in the original glycoprotein as compared to the modified glycoprotein, a determination of the likelihood that the modified glycoprotein is pathogenic or causative of a pathology may be made.

In some embodiments, the likelihood may be expressed as a probability. In some embodiments, the likelihood may be expressed as a pseudo-probability. In some embodiments, the likelihood may be expressed as a ratio or product of one or more probabilities or pseudo-probabilities. In some embodiments, the likelihood may be expressed as a product, quotient, sum or difference of one or more probabilities. In some embodiments, the likelihood may be expressed as an odds ratio. In some embodiments, the likelihood may be expressed as the logarithm of an odds ratio. In some embodiments, the likelihood may be expressed as a mathematical operation performed on one or more odds, odds ratios, log odds ratios, probabilities or pseudo-probabilities.

The predicted presence of the glycosylation feature at a glycosite in a sequence may be based on glycosylation feature structure, glycosylation feature composition, glycosylation feature length, glycosylation feature branching, sequence length, sequence composition, position of a monomer in the sequence, substitution of one or more monomers in the sequence, insertion of one or more monomers in the sequence, deletion of one or more monomers in the sequence, observed or predicted sequence secondary structure, observed or predicted sequence tertiary structure, observed or predicted sequence quaternary structure, or any combination thereof. The predicted presence may be based on a feature of the reference sequence. Alternatively or additionally, the predicted presence may be based on a feature of one or more of the variant sequences.

The method may comprise determining that the likelihood is above or below a cutoff or threshold value. Examples of threshold values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

In some embodiments, the likelihood is expressed as the logarithm of an odds ratio. The odds ratio may be determined from Fisher's exact test. The odds ratio may be determined by solving a set of generalized estimating equations. In some embodiments, the association score may be an IMR as described herein. In some embodiments, the IMR may be a generalized estimating equation (GEE) parameter. In some embodiments, the IMR may be an odds ratio as determined by Fisher's exact test.

The modified glycoprotein may differ from the reference glycoprotein in amino acid sequence. The difference in amino acid sequence may comprise a difference in one or more amino acid identities or positions. In some embodiments, the variant glycoprotein differs from the reference glycoprotein in the identity of an amino acid 1, 2, 3, 4, 5, 6, 7, 8, 9, 10, 11, 12, 13, 14, 15, 16, 17, 18, 19, 20 or more positions from the glycosite. In some embodiments, the variant glycoprotein differs from the reference glycoprotein in the identity of an amino acid 20, 19, 18, 17, 16, 15, 14, 13, 12, 11, 10, 9, 8, 7, 6, 5, 4, 3, 2, or fewer positions from the glycosite.

The modified glycoprotein may differ from the reference glycoprotein in length of amino acid sequence. The modified glycoprotein may have one or more insertions or deletions with respect to the reference glycoprotein. The modified glycopeptide may comprise 1, 2, 3, 4, 5, 6, 7, 8, 9, 10, 11, 12, 13, 14, 15, 16, 17, 18, 19, 20, or more insertions or deletions. The insertions or deletions may all be contiguous, or they may comprise one or more subsets across the sequence of the modified glycoprotein. The insertion or deletion may be proximal to a glycosite of the modified glycoprotein. In some embodiments, the insertion or deletion is within 1, 2, 3, 4, 5, 6, 7, 8, 9, 10, 11, 12, 13, 14, 15, 16, 17, 18, 19, 20, or more sites of the glycosite. In some embodiments, the insertion or deletion is within no more than 20, 19, 18, 17, 16, 15, 14, 13, 12, 11, 10, 9, 8, 7, 6, 5, 4, 3, 2, or fewer sites of the glycosite. In some embodiments, the insertion or deletion is in a site distal to the glycosite.

The modified glycoprotein may differ from the reference glycopeptide in one or more glycosylation features. The modified glycopeptide may differ from the reference glycopeptide in 1, 2, 3, 4, 5, 6, 7, 8, 9, 10, 20, 30, 40, 50, 60, 70, 80, 90, 100, or more glycosylation features. The one or more glycosylation features may be identical, or they may be independently distinct.

The methods and systems may be used to determine the likelihood of the individual having any disease or disorder known to be associated with a glycoprotein or glycosylation of a protein. In some embodiments, the disease or disorder comprises albinism, a prion disease, or Gaucher disease. In some embodiments, the prion disease comprises Creutzfeldt-Jakob Disease (CJD) or Gerstmann-Straussler disease (GSD).

### Pharmaceutical Compositions and Methods of Treatment

Also described herein are pharmaceutical compositions, wherein a pharmaceutical composition may comprise a modified glycoprotein as described herein or a fragment thereof. In some embodiments, a pharmaceutical composition may further comprise a pharmaceutically acceptable carrier, an excipient, or any combination thereof. A “pharmaceutically acceptable carrier or excipient” may comprise one or more molecular entities that do not materially affect the composition or change the active agent(s) contained therein, are physiologically tolerable, and do not typically produce an allergic reaction, or similar untoward reaction, when administered to a subject.

Also described herein are methods for treating a subject using a formulation or pharmaceutical composition as described herein. Also described herein are methods for prophylactic treatment of a subject using a formulation or pharmaceutical composition as described herein. Pharmaceutical compositions are formulated in a conventional manner using one or more pharmaceutically acceptable excipients that facilitate processing of the active compounds, i.e., modified glycoproteins or functional fragments thereof, into preparations that may be used pharmaceutically. Proper formulation is dependent upon the route of administration chosen. A summary of pharmaceutical compositions described herein may be found, for example, in Remington: The Science and Practice of Pharmacy, Nineteenth Ed. (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

Such methods may comprise administering to a subject an effective amount of the pharmaceutical composition or formulation. An effective amount may be determined, for example, based on the KD of a modified glycoprotein within the formulation or pharmaceutical composition, the bioavailability of a modified glycoprotein within the formulation or pharmaceutical composition, the route of administration of the formulation or pharmaceutical composition, other factors, or a combination thereof.

In some embodiments, a formulation or pharmaceutical composition may further comprise a second therapeutic. For example, a formulation or pharmaceutical composition may further comprise a pain reliever (e.g., ibuprofen or acetaminophen or any other suitable pain reliever), an antiviral compound (e.g., remdesivir or any other suitable antiviral compound), an antibiotic compound (e.g., azithromycin or any other suitable antibiotic compounds) or a steroid (e.g., dexamethasone, corticosteroids, cortisone, hydrocortisone, prednisone, or any other suitable steroids).

In some embodiments, a method may further comprise administering a pain reliever (e.g., ibuprofen or acetaminophen), an antiviral compound (e.g., remdesivir), an antibiotic compound (e.g., asithromycin) or a steroid (e.g., dexamethasone). In some embodiments, the second therapeutic compositions may be administered prior to the administration of the modified glycopeptides or the functional fragments thereof disclosed therein. In some embodiments, the second therapeutic compositions may be administered subsequent to the administration of the modified glycoproteins or the functional fragments thereof disclosed therein. In some embodiments, the second therapeutic compositions may be administered at the same time to the administration of the modified glycopeptides or the functional fragments thereof disclosed therein. In some embodiments, the second therapeutic may be conjugated to the modified glycopeptide.

### Computer Systems

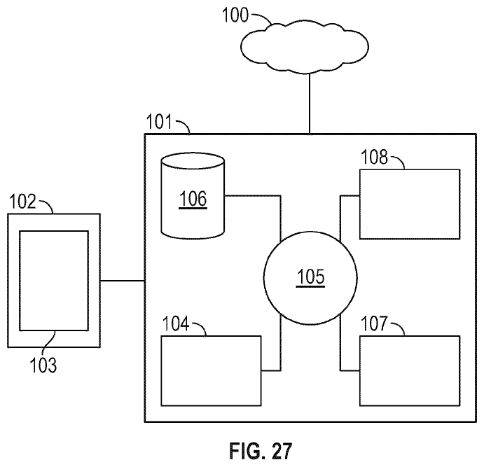

The present disclosure provides computer systems that are programmed to implement methods of the disclosure. FIG. 27 shows a computer system 101 that is programmed or otherwise configured to, for example, (i) train and test a trained algorithm, (ii) use the trained algorithm to predict the presence or absence of a glycosylation feature, (iii) use the trained algorithm to determine the effect of a sequence or structure modification on glycosylation, and (iv) use the trained algorithm to modify a glycopeptide.

The computer system 101 can regulate various aspects of analysis, calculation, and generation of the present disclosure, such as, for example, (i) training and testing a trained algorithm, (ii) using the trained algorithm to predict the presence or absence of a glycosylation feature, (iii) using the trained algorithm to determine the effect of a sequence or structure modification on glycosylation, and (iv) using the trained algorithm to modify a glycopeptide. The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 104 (e.g., random-access memory, read-only memory, flash memory), electronic storage unit 106 (e.g., hard disk), communication interface 108 (e.g., network adapter) for communicating with one or more other systems, and peripheral devices 107, such as cache, other memory, data storage and/or electronic display adapters. The memory 104, storage unit 106, interface 108 and peripheral devices 107 are in communication with the CPU 105 through a communication bus (solid lines), such as a motherboard. The storage unit 106 can be a data storage unit (or data repository) for storing data. The computer system 101 can be operatively coupled to a computer network (“network”) 100 with the aid of the communication interface 108. The network 100 can be the Internet, an internet and/or extranet, or an intranet and/or extranet that is in communication with the Internet.

In some embodiments, the network 100 is a telecommunication and/or data network. The network 100 can include one or more computer servers, which can enable distributed computing, such as cloud computing. For example, one or more computer servers may enable cloud computing over the network 100 (“the cloud”) to perform various aspects of analysis, calculation, and generation of the present disclosure, such as, for example, (i) training and testing a trained algorithm, (ii) using the trained algorithm to predict the presence or absence of a glycosylation feature, (iii) using the trained algorithm to determine the effect of a sequence or structure modification on glycosylation, and (iv) using the trained algorithm to modify a glycopeptide. Such cloud computing may be provided by cloud computing platforms such as, for example, Amazon Web Services (AWS), Microsoft Azure, Google Cloud Platform, and IBM cloud. In some embodiments, the network 100, with the aid of the computer system 101, can implement a peer-to-peer network, which may enable devices coupled to the computer system 101 to behave as a client or a server.

The CPU 105 may comprise one or more computer processors and/or one or more graphics processing units (GPUs). The CPU 105 can execute a sequence of machine-readable instructions, which can be embodied in a program or software. The instructions may be stored in a memory location, such as the memory 104. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some embodiments, the circuit is an application specific integrated circuit (ASIC).

The storage unit 106 can store files, such as drivers, libraries and saved programs. The storage unit 106 can store user data, e.g., user preferences and user programs. In some embodiments, the computer system 101 can include one or more additional data storage units that are external to the computer system 101, such as located on a remote server that is in communication with the computer system 101 through an intranet or the Internet.

The computer system 101 can communicate with one or more remote computer systems through the network 100. For instance, the computer system 101 can communicate with a remote computer system of a user. Examples of remote computer systems include personal computers (e.g., portable PC), slate or tablet PC's (e.g., AppleR iPad, SamsungR Galaxy Tab), telephones, Smart phones (e.g., Apple® iPhone, Android-enabled device, Blackberry®), or personal digital assistants. The user can access the computer system 101 via the network 100.

Methods as described herein can be implemented by way of machine (e.g., computer processor) executable code stored on an electronic storage location of the computer system 101, such as, for example, on the memory 104 or electronic storage unit 106. The machine executable or machine readable code can be provided in the form of software. During use, the code can be executed by the processor 105. In some embodiments, the code can be retrieved from the storage unit 106 and stored on the memory 104 for ready access by the processor 105. In some situations, the electronic storage unit 106 can be precluded, and machine-executable instructions are stored on memory 104.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Embodiments of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, or disk drives, which may provide non-transitory storage at any time for the software programming. All or portions of the software may at times be communicated through the Internet or various other teleconmmunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 102 that comprises a user interface (UI) 103 for providing, for example, (i) a visual display indicative of training and testing a trained algorithm, (ii) a visual display indicative of using the trained algorithm to predict the presence or absence of a glycosylation feature, (iii) a visual display indicative of using the trained algorithm to determine the effect of a sequence or structure modification on glycosylation, and (iv) a visual display indicative of using the trained algorithm to modify a glycopeptide. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 105. The algorithm can, for example, (i) train and test a trained algorithm, (ii) use the trained algorithm to predict the presence or absence of a glycosylation feature, (iii) use the trained algorithm to determine the effect of a sequence or structure modification on glycosylation, and (iv) use the trained algorithm to modify a glycopeptide

### LIST OF EMBODIMENTS

The following list of embodiments of the invention are to be considers as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A method for determining the effect of a variation of a reference sequence on glycosylation of a glycosite in the reference sequence, the method comprising:
(a) providing a plurality of sequences comprising (1) the reference sequence and optionally the associated three-dimensional structure, and (2) a plurality of variant sequences, and optionally the associated three-dimensional structures, having one or more amino acid substitution as compared to the reference sequence; and
(b) for each of the plurality of variant sequences, and optionally associated three-dimensional structures: applying a trained algorithm to calculate the predicted presence of a glycosylation feature at the glycosite of each variant sequence and optional associated three-dimensional structure based at least on the amino acid sequence and the optional associated three-dimensional structure of the variant sequence; thereby determining the effect of the variation of the reference sequence on glycosylation of the glycosite.

Embodiment 2. The method of embodiment 1, wherein the glycosylation feature is a specific monosaccharide or a polysaccharide epitope.

Embodiment 3. The method of embodiment 2, wherein the specific monosaccharide is mannose, sialic acid, fucose, D-glucose (Glc), D-galactose (Gal), N-acetylglucosamine (GlcNAc), N-acetylgalactosamine (GalNAc), D-mannose (Man), N-acetylneuraminic acid (Neu5Ac), N-glycolylneuraminic acid (Neu5Gc), neuraminic acid (Neu), 2-keto-3-deoxynononic acid or 3-deoxy-D-glycero-D-galacto-nonulosonic acid (KDN), 3-deoxy-D-manno-2 octulopyranosylonic acid (Kdo), D-galacturonic acid (GalA), L-iduronic acid (IdoA), L-rhamnose (Rha), L-fucose (Fuc), D-Xylose (Xyl), D-ribose (Rib), L-arabinofuranose (Araf), D-glucuronic acid (GlcA), D-allose (All), D-apiose (Api), D-fructofuranose (Fruf), ascarylose (Asc), or ribitol (Rbo), or a combination thereof.

Embodiment 4. The method of embodiment 2, wherein the polysaccharide epitope is high-mannose, sialylation, fucosylation, hybrid, complexity, core or distally fucosylation, terminal sialylation, terminal galactosylation, terminal GlcNAc-ylation, GlcNAc-bisection, or poly-sialylation, or a glycosylation feature listed in Table 1, or a combination thereof.

Embodiment 5. The method of any one of embodiments 1-4, wherein the glycosylation feature is an increase in high-mannose in the variant sequence as compared to the reference sequence.

Embodiment 6. The method of any one of embodiments 1-4, wherein the glycosylation feature is decrease in high-mannose in the variant sequence as compared to the reference sequence.

Embodiment 7. The method of any one of embodiments 1-4, wherein the glycosylation feature is an increase in sialylation in the variant sequence as compared to the reference sequence.

Embodiment 8. The method of any one of embodiments 1-4, wherein the glycosylation feature is decrease in sialylation in the variant sequence as compared to the reference sequence.

Embodiment 9. The method of any one of embodiments 1-4, wherein the glycosylation feature is an increase in fucosylation in the variant sequence as compared to the reference sequence.

Embodiment 10. The method of any one of embodiments 1-4, wherein the glycosylation feature is decrease in fucosylation in the variant sequence as compared to the reference sequence.

Embodiment 11. The method of any one of embodiments 1-10, wherein the predicted presence that the glycosite of each variant sequence will have a glycosylation feature is determined at least based on the identity of one or more amino acid sequences varied as compared to the reference sequence.

Embodiment 12. The method of any one of embodiments 1-11, wherein the pseudo-probability that the glycosite of each variant sequence will have a glycosylation feature is determined at least based on the position of one or more amino acid sequences varied as compared to the reference sequence.

Embodiment 13. The method of embodiment 12, wherein the position is the distance from the glycosite.

Embodiment 14. The method of any one of embodiments 1-13, wherein each variant sequence has at least one amino acid substitution as compared to the reference sequence.

Embodiment 15. The method of any one of embodiments 1-13, wherein each variant sequence has at least two amino acid substitution as compared to the reference sequence.

Embodiment 16. The method of any one of embodiments 1-15, wherein the glycosite comprises a glycan-bound arginine, asparagine, serine, threonine, or tyrosine.

Embodiment 17. The method of embodiment 16, wherein the glycosite further comprises one or more amino acids N-terminal to the glycan bound arginine, asparagine, serine, threonine, or tyrosine.

Embodiment 18. The method of embodiment 16 or embodiment 17, wherein the glycosite further comprises one or more amino acids C-terminal to the glycan bound arginine, asparagine, serine, threonine, or tyrosine.

Embodiment 19. The method of any one of embodiments 1-18, wherein the sequence of a first variant sequence is comprised within a peptide.

Embodiment 20. The method of embodiment 19, further comprising administering a therapeutically effective amount of the peptide based at least in part on determining the effect of the variation of the reference sequence on glycosylation of the glycosite.

Embodiment 21. A computer system comprising a digital processing device comprising at least one processor, an operating system configured to perform executable instructions, a memory, and a computer program including instructions executable by the digital processing device to create an application for determining the effect of a variation of a reference sequence and optionally the associated three-dimensional structure on glycosylation of a glycosite in the reference sequence, the application comprising: a module programmed to, for each of a plurality of variant sequences, and optionally the associated three-dimensional structures, having one or more amino acid substitution as compared to the reference sequence, apply a trained algorithm to calculate the predicted presence of a glycosylation feature at the glycosite of each variant sequence based at least on the amino acid sequence and the optional associated three-dimensional structure of the variant sequence.

Embodiment 22. A non-transitory computer-readable medium comprising machine-executable code that, upon execution by one or more computer processors, implements a method for determining the effect of a variation of a reference sequence on glycosylation of a glycosite in the reference sequence, the method comprising:
(a) providing a plurality of sequon sequences comprising (1) the reference sequence and optionally the associated three-dimensional structure, and (2) a plurality of variant sequences, and optionally the associated three-dimensional structures, having one or more amino acid substitution as compared to the reference sequence; and
(b) for each of the plurality of variant sequences, and optionally associated three-dimensional structures: applying a trained algorithm to calculate the predicted presence of a glycosylation feature at the glycosite of each variant sequence and optional associated three-dimensional structure based at least on the amino acid sequence and the optional associated three-dimensional structure of the variant sequence; thereby determining the effect of the variation of the reference sequence on glycosylation of the glycosite.

Embodiment 23. A system for determining the effect of a variation of a reference sequence and optionally the associated three-dimensional structure on glycosylation of a glycosite in the reference sequence, the system comprising: a database comprising a plurality of sequences comprising (1) the reference sequence and optionally the associated three-dimensional structure, and (2) a plurality of variant sequences, and optionally the associated three-dimensional structures, having one or more amino acid substitution as compared to the reference sequence; and one or more computer processors operatively coupled to the database, wherein the one or more computer processors are individually or collectively programmed to: for each of the plurality of variant sequences, and optionally associated three-dimensional structures: applying a trained algorithm to calculate the predicted presence of a glycosylation feature at the glycosite of each variant sequence and optionally associated three-dimensional structure based at least on the amino acid sequence and the optional associated three-dimensional structure of the variant sequence; thereby determining the effect of the variation of the reference sequence on glycosylation of the glycosite.

Embodiment 24. The system of any one of embodiments 21-23, wherein the glycosylation feature is a specific monosaccharide or a polysaccharide epitope.

Embodiment 25. The system of embodiment 24, wherein the specific monosaccharide is mannose, sialic acid, fucose, D-glucose (Glc), D-galactose (Gal), N-acetylglucosamine (GlcNAc), N-acetylgalactosamine (GalNAc), D-mannose (Man), N-acetylneuraminic acid (Neu5Ac), N-glycolylneuraminic acid (Neu5Gc), neuraminic acid (Neu), 2-keto-3-deoxynononic acid or 3-deoxy-D-glycero-D-galacto-nonulosonic acid (KDN), 3-deoxy-D-manno-2 octulopyranosylonic acid (Kdo), D-galacturonic acid (GalA), L-iduronic acid (IdoA), L-rhamnose (Rha), L-fucose (Fuc), D-xylose (Xyl), D-ribose (Rib), L-arabinofuranose (Araf), D-glucuronic acid (GlcA), D-allose (All), D-apiose (Api), D-fructofuranose (Fruf), ascarylose (Asc), or ribitol (Rbo), or a combination thereof.

Embodiment 26. The system of embodiment 24, wherein the polysaccharide epitope is high-mannose, sialylation, fucosylation, hybrid, complexity, core or distally fucosylation, terminal sialylation, terminal galactosylation, terminal GlcNAc-ylation, GlcNAc-bisection, or poly-sialylation, or a glycosylation feature listed in Table 1, or a combination thereof.

Embodiment 27. The system of any one of embodiments 21-26, wherein the glycosylation feature is an increase in high-mannose in the variant sequence as compared to the reference sequence.

Embodiment 28. The system of any one of embodiments 21-26, wherein the glycosylation feature is decrease in high-mannose in the variant sequence as compared to the reference sequence.

Embodiment 29. The system of any one of embodiments 21-26, wherein the glycosylation feature is an increase in sialylation in the variant sequence as compared to the reference sequence.

Embodiment 30. The system of any one of embodiments 21-26, wherein the glycosylation feature is decrease in sialylation in the variant sequence as compared to the reference sequence.

Embodiment 31. The system of any one of embodiments 21-26, wherein the glycosylation feature is an increase in fucosylation in the variant sequence as compared to the reference sequence.

Embodiment 32. The system of any one of embodiments 21-26, wherein the glycosylation feature is decrease in fucosylation in the variant sequence as compared to the reference sequence.

Embodiment 33. The system of any one of embodiments 21-32, wherein the pseudo-probability that the glycosite of each variant sequence will have a glycosylation feature is determined at least based on the identity of one or more amino acid sequences varied as compared to the reference sequence.

Embodiment 34. The system of any one of embodiments 21-33, wherein the pseudo-probability that the glycosite of each variant sequence will have a glycosylation feature is determined at least based on the position of one or more amino acid sequences varied as compared to the reference sequence.

Embodiment 35. The system of embodiment 34, wherein the position is the distance from the glycosite.

Embodiment 36. The system of any one of embodiments 21-35, wherein each variant sequence has one amino acid substitution as compared to the reference sequence.

Embodiment 37. The system of any one of embodiments 21-35, wherein each variant sequence has at least two amino acid substitution as compared to the reference sequence.

Embodiment 38. The system of any one of embodiments 21-37, wherein the glycosite comprises an arginine, asparagine, serine, threonine, or tyrosine.

Embodiment 39. The system of embodiment 38, wherein the glycosite further comprises one or more amino acids N-terminal to the glycan bound arginine, asparagine, serine, threonine, or tyrosine.

Embodiment 40. The system of embodiment 38 or embodiment 39, wherein the glycosite further comprises one or more amino acids C-terminal to the glycan bound arginine, asparagine, serine, threonine, or tyrosine.

Embodiment 41. The system of any one of embodiments 21-40, wherein the sequence of a variant sequence is comprised within a peptide.

Embodiment 42. A method of treatment comprising administering to a subject in need thereof a therapeutically effective amount of the peptide of embodiment 41.

Embodiment 43. A method of modifying a reference glycopeptide to alter a glycosylation feature of a glycosite of the reference glycopeptide to produce a modified glycopeptide, the method comprising: identifying a predicted presence of the glycosylation feature at a glycosite of a modified glycopeptide, which modified glycopeptide comprises one or more amino acid substitutions to a sequence of the reference glycopeptide, and generating the modified glycopeptide having the one or more amino acid substitutions in the sequence of the reference glycopeptide if the predicted presence is at least a threshold predicted presence.

Embodiment 44. The method of embodiment 43, wherein the threshold pseudo-probability is about 50%, 60%, 70%, 80%, 90%, or higher.

Embodiment 45. The method of embodiment 43 or embodiment 44, wherein the predicted presence is determined using a trained algorithm.

Embodiment 46. The method of any one of embodiments 43-45, wherein the predicted presence is determined at least based on the identity of one or more amino acids varied as compared to the reference sequence.

Embodiment 47. The method of any one of embodiments 43-45, wherein the predicted presence is determined at least based on the position of one or more amino acids varied as compared to the reference sequence.

Embodiment 48. The method of embodiment 47, wherein the position is the distance from the glycosite.

Embodiment 49. A method of modifying a reference glycopeptide to alter a glycosylation feature of a glycosite of the reference glycopeptide to produce a modified glycopeptide, the method comprising: substituting one or more amino acids within 15 amino acids of the glycosite to generate the modified glycopeptide.

Embodiment 50. The method of any one of embodiments 43-49, wherein the glycosylation feature is high-mannose, sialylation, fucosylation, or a combination thereof.

Embodiment 51. The method of any one of embodiments 43-50, wherein the glycosylation feature is an increase in high-mannose in the modified glycopeptide as compared to the reference glycopeptide.

Embodiment 52. The method of any one of embodiments 43-50, wherein the glycosylation feature is decrease in high-mannose in the modified glycopeptide as compared to the reference glycopeptide.

Embodiment 53. The method of any one of embodiments 43-50, wherein the glycosylation feature is an increase in sialylation in the modified glycopeptide as compared to the reference glycopeptide.

Embodiment 54. The method of any one of embodiments 43-50, wherein the glycosylation feature is decrease in sialylation in the modified glycopeptide as compared to the reference glycopeptide.

Embodiment 55. The method of any one of embodiments 43-50, wherein the glycosylation feature is an increase in fucosylation in the modified glycopeptide as compared to the reference glycopeptide.

Embodiment 56. The method of any one of embodiments 43-50, wherein the glycosylation feature is decrease in fucosylation in the modified glycopeptide as compared to the reference glycopeptide.

Embodiment 57. The method of any one of embodiments 43-56, wherein the glycosite comprises an arginine, asparagine, serine, threonine, or tyrosine.

Embodiment 58. The method of any one of embodiments 43-57, further comprising administering a therapeutically effective amount of the modified glycopeptide to a subject in need thereof based at least in part on the altered glycosylation feature of the modified glycopeptide.

Embodiment 59. A modified glycopeptide having a first glycosylation feature that is different from a reference glycosylation feature of a glycosite of a reference glycoprotein, wherein the modified glycopeptide has one or more amino acid substitutions in a sequence comprising the glycosite as compared to the reference glycoprotein.

Embodiment 60. The modified glycopeptide of embodiment 59, wherein the one or more amino acid substitutions is positioned within 1, 2, 3, 4, 5, 6, 7, 8, 9, 10, 11, 12, 13, 14, 15, 16, 17, 18, 19, 20, 21, 22, 23, 24, or 25 amino acids of the glycosite; or wherein the one or more amino acid substitutions is positioned within a sequon comprising the glycosite.

Embodiment 61. The modified glycopeptide of embodiment 59 or embodiment 60, wherein the first glycosylation feature is a specific monosaccharide or a polysaccharide epitope.

Embodiment 62. The modified glycopeptide of embodiment 61, wherein the specific monosaccharide is mannose, sialic acid, fucose, D-glucose (Glc), D-galactose (Gal), N-acetylglucosamine (GlcNAc), N-acetylgalactosamine (GalNAc), D-mannose (Man), N-acetylneuraminic acid (Neu5Ac), N-glycolylneuraminic acid (Neu5Gc), neuraminic acid (Neu), 2-keto-3-deoxynononic acid or 3-deoxy-D-glycero-D-galacto-nonulosonic acid (KDN), 3-deoxy-D-manno-2 octulopyranosylonic acid (Kdo), D-galacturonic acid (GalA), L-iduronic acid (IdoA), L-rhamnose (Rha), L-fucose (Fuc), D-xylose (Xyl), D-ribose (Rib), L-arabinofuranose (Araf), D-glucuronic acid (GlcA), D-allose (All), D-apiose (Api), D-fructofuranose (Fruf), ascarylose (Asc), or ribitol (Rbo), or a combination thereof.

Embodiment 63. The modified glycopeptide of embodiment 61, wherein the polysaccharide epitope is high-mannose, sialylation, fucosylation, hybrid, complexity, core or distally fucosylation, terminal sialylation, terminal galactosylation, terminal GlcNAc-ylation, GlcNAc-bisection, or poly-sialylation, or a glycosylation feature listed in Table 1, or a combination thereof.

Embodiment 64. The modified glycopeptide of any one of embodiments 59-63, wherein the first glycosylation feature is an increase in high-mannose in the modified glycopeptide as compared to the reference glycopeptide.

Embodiment 65. The modified glycopeptide of any one of embodiments 59-63, wherein the first glycosylation feature is decrease in high-mannose in the modified glycopeptide as compared to the reference glycopeptide.

Embodiment 66. The modified glycopeptide of any one of embodiments 59-63, wherein the first glycosylation feature is an increase in sialylation in the modified glycopeptide as compared to the reference glycopeptide.

Embodiment 67. The modified glycopeptide of any one of embodiments 59-63, wherein the first glycosylation feature is decrease in sialylation in the modified glycopeptide as compared to the reference glycopeptide.

Embodiment 68. The modified glycopeptide of any one of embodiments 59-63, wherein the first glycosylation feature is an increase in fucosylation in the modified glycopeptide as compared to the reference glycopeptide.

Embodiment 69. The modified glycopeptide of any one of embodiments 59-63, wherein the first glycosylation feature is decrease in fucosylation in the modified glycopeptide as compared to the reference glycopeptide.

Embodiment 70. The modified glycopeptide of any one of embodiments 59-69, wherein the glycosite comprises an arginine, asparagine, serine, threonine, or tyrosine.

Embodiment 71. A method comprising administering a therapeutically effective amount of the modified glycopeptide of any one of embodiments 59-70 to a subject in need thereof based at least in part on the first glycosylation feature of the modified glycopeptide.

Embodiment 72. A method for determining the likelihood that one or more glycans from a plurality of candidate glycans will be found at a glycosite of a sequence, the method comprising:
(a) providing the sequence (and optionally the associated three-dimensional structure) and the plurality of candidate glycans;
(b) for each of the plurality of candidate glycans: applying a trained algorithm to calculate a predicted presence for each glycan at the glycosite of the sequence determined at least based on one or more amino acids in the sequence (and optionally the associated three-dimensional structure); and
(c) computer processing the predicted presence for each of the plurality of candidate glycans to determine the likelihood that the one or more glycans will be found at the glycosite of the sequence.

Embodiment 73. The method of embodiment 72, wherein the one or more glycans comprises at least one glycan of Table 1.

Embodiment 74. The method of embodiment 72 or embodiment 73, wherein the predicted presence of the glycan at the glycosite is determined at least based on the identity of the one or more amino acids in the sequence.

Embodiment 75. The method of embodiment 72 or embodiment 73, wherein the predicted presence of the glycan at the glycosite is determined at least based on the position of the one or more amino acids in the sequence.

Embodiment 76. The method of embodiment 72 or embodiment 73, wherein the predicted presence of the glycan at the glycosite is determined at least based on the identity and position of the one or more amino acids in the sequence.

Embodiment 77. The method of any one of embodiments 72-76, wherein the one or more amino acids in the sequence is located within 1, 2, 3, 4, 5, 6, 7, 8, 9, or 10 amino acids of the glycosite.

Embodiment 78. The method of any one of embodiments 72-77, wherein the glycosite comprises an arginine, asparagine, serine, threonine, or tyrosine.

Embodiment 79. The method of embodiment 78, wherein the glycosite further comprises one or more amino acids N-terminal to the glycan bound arginine, asparagine, serine, threonine, or tyrosine.

Embodiment 80. The method of embodiment 78 or embodiment 79, wherein the glycosite further comprises one or more amino acids C-terminal to the glycan bound arginine, asparagine, serine, threonine, or tyrosine.

Embodiment 81. The method of any one of embodiments 72-80, wherein the sequence is comprised within a peptide.

Embodiment 82. The method of embodiment 81, wherein precursors of the one or more glycans are glycans present in a host cell during production of the peptide.

Embodiment 83. The method of embodiment 81 or embodiment 82, wherein precursors of the one or more glycans are glycans present in a host cell medium during production of the peptide.

Embodiment 84. The method of any one of embodiments 81-83, further comprising administering a therapeutically effective amount of the peptide based at least in part on determining whether the one or more glycans will be found at the glycosite of the sequence.

Embodiment 85. A computer system comprising a digital processing device comprising at least one processor, an operating system configured to perform executable instructions, a memory, and a computer program including instructions executable by the digital processing device to create an application for determining the likelihood that one or more glycans from a plurality of candidate glycans will be found at a glycosite of a sequence, the application comprising:
(a) a module programmed to, for each of the plurality of candidate glycans, apply a trained algorithm to calculate a predicted presence for each glycan at the glycosite of the sequence determined at least based on one or more amino acids in the sequence (and optionally the associated three-dimensional structure of the sequence) to generate a plurality of predicted presences; and
(b) a processing module programmed to process the plurality of predicted presences to determine the likelihood that the one or more glycans will be found at the glycosite of the sequence.

Embodiment 86. A non-transitory computer-readable medium comprising machine-executable code that, upon execution by one or more computer processors, implements a method for determining the likelihood that one or more glycans from a plurality of candidate glycans will be found at a glycosite of a sequence, the method comprising:
(a) providing the sequence (and optionally the associated three-dimensional structure) and the plurality of candidate glycans;
(b) for each of the plurality of candidate glycans: applying a trained algorithm to calculate a predicted presence for each glycan at the glycosite of the sequence determined at least based on one or more amino acids in the sequence (and optionally the associated three-dimensional structure); and
(c) computer processing the predicted presence for each of the plurality of candidate glycans to determine the likelihood that the one or more glycans will be found at the glycosite of the sequence.

Embodiment 87. A system for determining the likelihood that one or more glycans from a plurality of candidate glycans will be found at a glycosite of a sequence, the system comprising: a database comprising the plurality of candidate glycans; and one or more computer processors operatively coupled to the database, wherein the one or more computer processors are individually or collectively programmed to:
(a) for each of the plurality of candidate glycans: apply a trained algorithm to calculate a predicted presence for each glycan at the glycosite of the sequence determined at least based on one or more amino acids in the sequence (and optionally the associated three-dimensional structure of the sequence); and
(b) process the predicted presence for each of the plurality of candidate glycans to determine the likelihood that the one or more glycans will be found at the glycosite of the sequence.

Embodiment 88. The system of any one of embodiments 85-87, wherein the one or more glycans comprises at least one glycan of Table 1.

Embodiment 89. The system of any one of embodiments 85-88, wherein the predicted presence for the glycan at the glycosite of the sequence is determined at least based on the identity of the one or more amino acids in the sequence.

Embodiment 90. The system of any one of embodiments 85-89, wherein the predicted presence for the glycan at the glycosite of the sequence is determined at least based on the position of the one or more amino acids in the sequence.

Embodiment 91. The system of any one of embodiments 85-90, wherein the predicted presence for the glycan at the glycosite of sequence is determined at least based on the identity and position of the one or more amino acids in the sequence.

Embodiment 92. The system of any one of embodiments 85-91, wherein the one or more amino acids in the sequence is located within 1, 2, 3, 4, 5, 6, 7, 8, 9, or 10 amino acids of the glycosite.

Embodiment 93. The system of any one of embodiments 85-92, wherein the glycosite comprises an arginine, asparagine, serine, threonine, or tyrosine.

Embodiment 94. The system of embodiment 93, wherein the glycosite further comprises one or more amino acids N-terminal to the glycan bound arginine, asparagine, serine, threonine, or tyrosine.

Embodiment 95. The system of embodiment 93 or embodiment 94, wherein the glycosite further comprises one or more amino acids C-terminal to the glycan bound arginine, asparagine, serine, threonine, or tyrosine.

Embodiment 96. The system of any one of embodiments 85-95, wherein the sequence is comprised within a peptide.

Embodiment 97. The system of embodiment 96, wherein precursors of the one or more glycans are glycans present in a host cell during production of the peptide.

Embodiment 98. The system of embodiment 96 or embodiment 97, wherein precursors of the one or more glycans are glycans present in a host cell medium during production of the peptide.

### EXAMPLES

### Example 1. Discovery of Templating Principles for Glycoprotein Synthesis

Glycan biosynthesis, unlike DNA, RNA, and protein biosynthesis, has been described previously as “non-templated.” Through structural analysis of site-specific glycosylation data, described herein are protein-sequence and structural features that may predict specific glycan structures. Differences in sequence-predicted glycosylation, which may be referred to as “glycoimpact” herein, increase when the PAM and BLOSUM substitution matrices disagree. High-glycoimpact amino acids may also co-evolve with glycosites. Similarly, high-glycoimpact ClinVar variants observed close to glycosites may be associated with glycan-actuated diseases such as in Albinism and prion disease. More broadly, glycoimpact may predict disagreement between multiple pathogenicity predictions (e.g. VEP).

DNA, RNA, and protein sequences may be predictably templated by DNA and codon templates, respectively. Distinctly, glycosylation is often described as a non-templated process. However, protein primary sequence influences glycan diversity and identity. In the initial description of the N-glycosylation sequon, glycans were found to covalently bind asparagine (N) residues with a downstream (N+2) serine (S) or threonine (T) separated by any amino acid (AA) but proline (NXS/T). Variation at N+1 may impact glycan complexity. Glycosylation of a sequon ending in threonine is approximately 40 times more efficient than those ending in serine. Upstream of the glycosite, a phenylalanine to alanine substitution in human IgG3 increased bigalactose structures with a core-fucose. Additionally, influenza evolves glycosylation sites to evade immune detection. Tools like GlycoSiteAlign and mutagenesis studies have offered expansions of the primary sequon structure including the enhanced aromatic sequon; an aromatic residue upstream of the glycosite (N-2) that can influence glycan complexity with a variable impact given the N+1 variation.

Glycoconjugate influence may be observed beyond the primary sequence. Secondary structures, including β-sheets and α-helixes, may influence glycosylation.

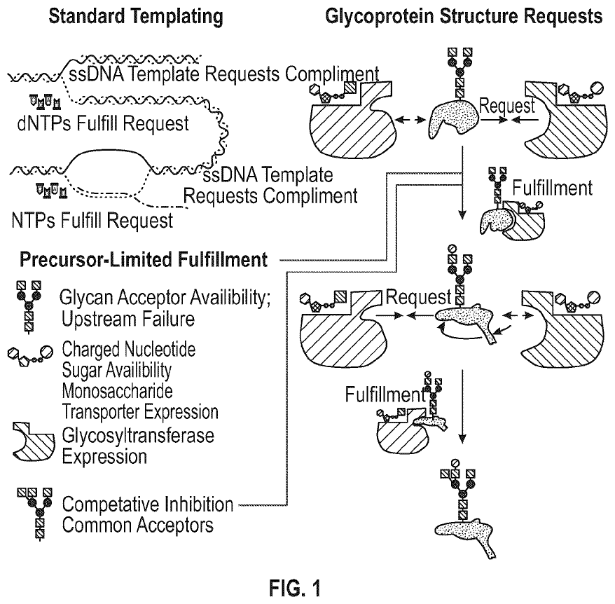

Glycosylation may be determined by both cellular metabolic environments and site-specific, glycoconjugate-defined microenvironments. However, these have not been consolidated in a clear and interoperable mapping from the genome to the glycome. As described herein, glycan biosynthesis was integrated with site-specific protein structure features to generalize the template for glycosylation. Fundamental to this is precursor-limited templating, i.e., a templated process wherein the substrate requested is not always available; thus, the template is difficult to observe without biosynthetic knowledge-defining all possible intermediates and the final possible glycans. A glycosylation template may be described as a mapping from “glycoimpactful” protein structure to expected glycan substructures. These glycoimpact relations were validated by comparison to evolutionary substitution matrices and mutation pathogenicity scores. Further, pathogenic glycoimpactful mutations were found enriched near glycosylation sites. Finally, glycoimpact was used to accurately predict changes in glycan complexity, galactosylation, sialylation and functional glycosylation. A model of glycosylation consistent with this work is illustrated in FIG. 1.

### Results

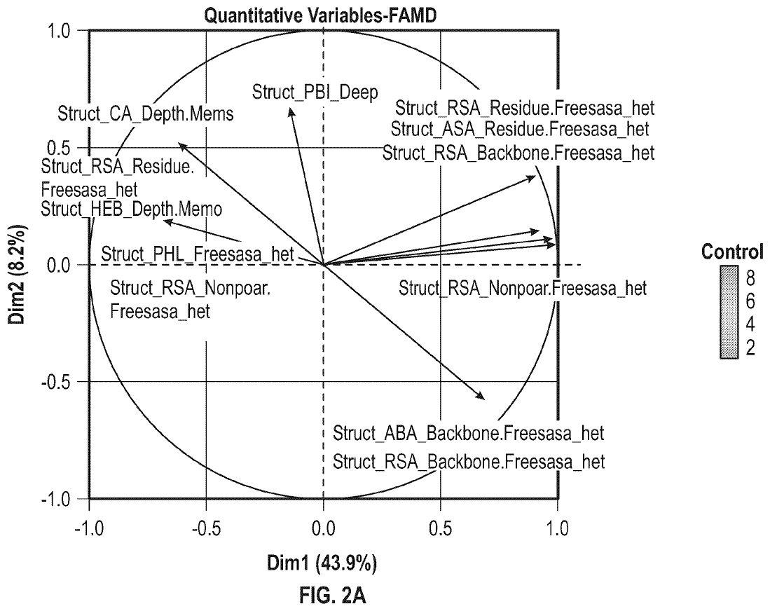

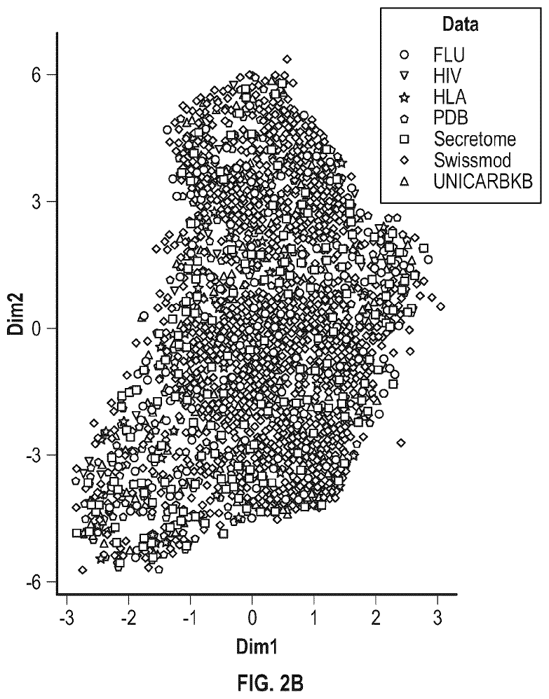

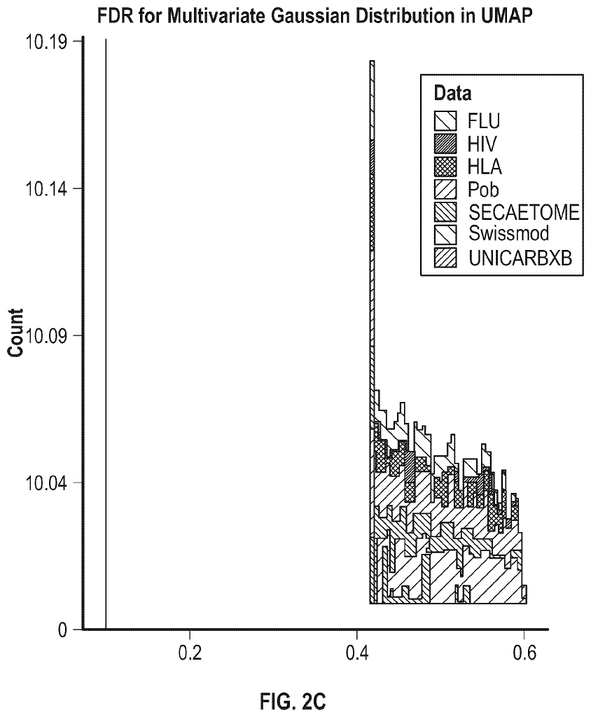

First, glycan structure was tested to see if it correlates with protein structure. To do this, the Protein-Glycan Enriched Structure Database (PGES-DB), a compendium of glycosylation sites on proteins and their experimentally measured glycans, was built leveraging the UnicarbKB and GlyConnect databases. Glycan structures were decomposed into their substructures that describe intermediates in their biosynthesis (termed “substructures”) using GlyCompare. Features of the glycosite-proximal protein structure were annotated using the Structural System Biology (SSBio) toolkit. Briefly, PGES-DB contains protein structures (empirical (PDB), curated homology models (SWISSMOD) and ab initio homology models (1-TASSER)). This includes 98 glycoproteins with N-glycosylation sites and 38 glycoproteins with O-glycosylation sites including 3,563 N-glycosylation and 700 O-glycosylation events. First the glycosite-structure annotation was checked to be representative of typical variation in glycosites; the structure-annotated glycosites from the input database were used to train a dimensionality reduction. The results of this Factor Analysis with Mixed Data (FAMD) are shown in FIG. 2A. Glycosites throughout the human secretome were then projected into the reduced space, as illustrated in FIG. 2B. Using a multivariate Gaussian, the probability that each non-input glycosite was within the distribution of input glycosite variation was determined. After a False Discovery Rate (FDR) correction, no outlying glycosite structures were found, as shown in FIG. 2C, indicating that the PGES-DB glycosites are representative of the space of glycosite structure.

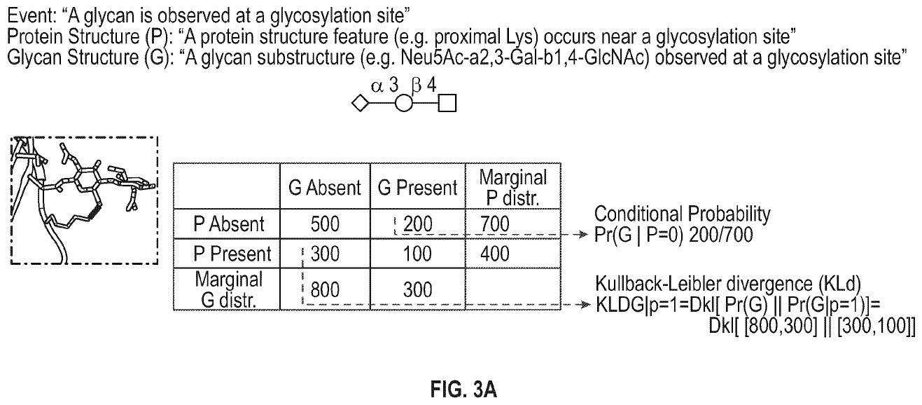

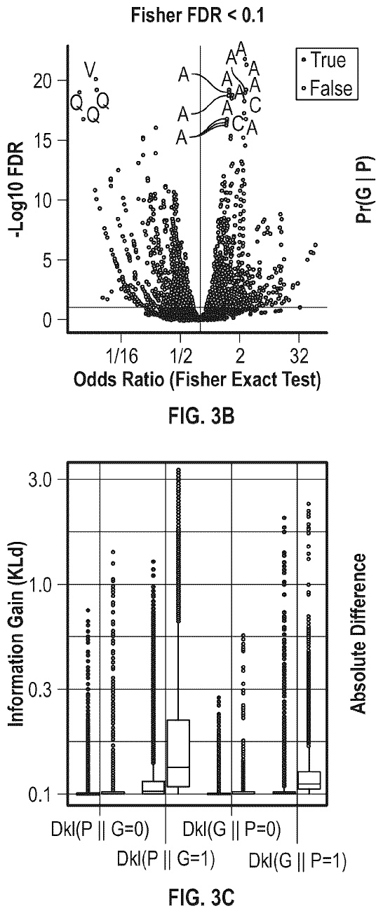

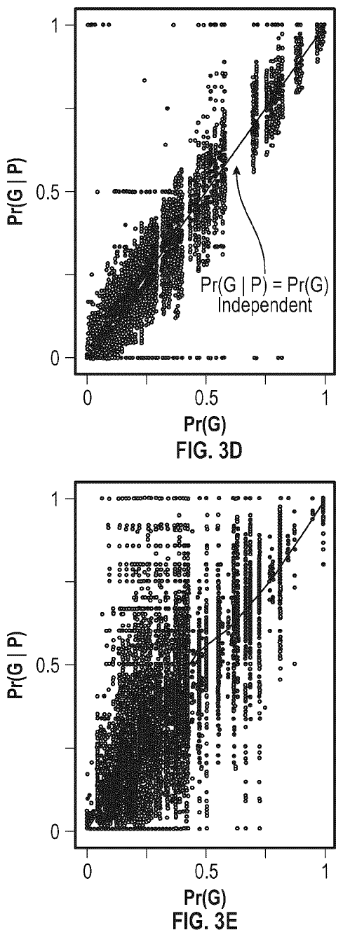

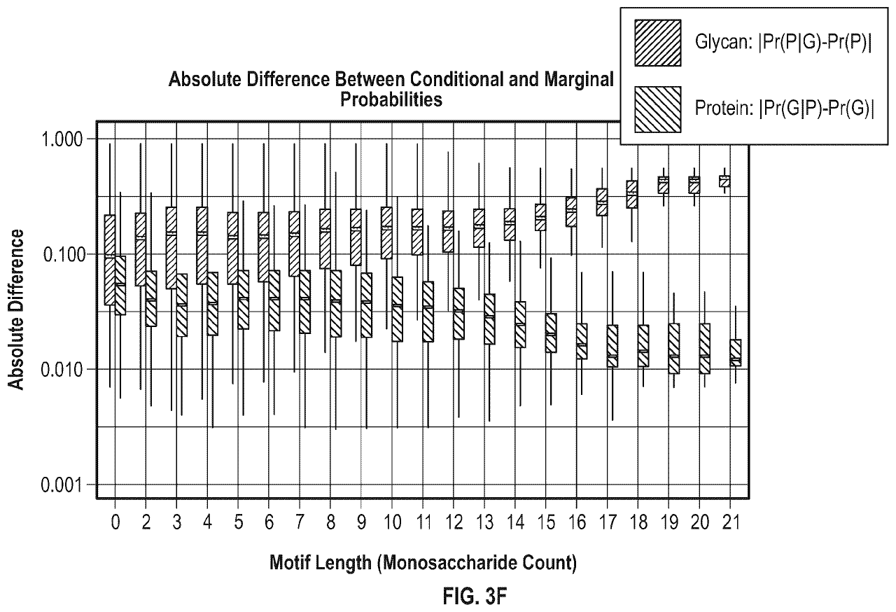

With a representative mapping of the glycosite structure manifold, the associations between glycan substructures (e.g., tri-mannose) and glycosite-proximal protein features (e.g., proximal tyrosine) within PGES-DB were examined (FIG. 3A). The Fisher exact test was used to estimate odds ratios (OR) for intermolecular relations (IMR), which may comprise quantitative associations between glycan substructures and protein features. To further probe these IMR, the augmentation to conditional-marginal probability difference (CMd) and Kullback-Leibler divergence (KLd) between conditional and marginal distributions of protein structure when glycan structure was fixed (G=1 or G=0) and glycan structure when protein structure was fixed (P=1 or P=0) were investigated.

Of 259,114 potential substructure-IMRs, 50,842 relationships that were substantial (|Fisher-OR|>0.1) and significant (Fisher-FDR<0.1) were found, and 10,111 of 26,404 substantial and significant motif-IMRs. Of the 10,111 selected motif-IMRs, 9,296 and 815 showed significant correlation and anticorrelation, respectively. Representative significant IMRs included correlations with glycosylation-site-proximal (within 5 Å) alanine and cysteine and anticorrelations with proximal arginine and valine (FIG. 3B). To further probe the motif-IMRs, the CMd and KLd when either protein structure or glycan structure was known were investigated. KLd was low for significant IMRs (Fisher-FDR<0.1) when protein structure or glycan structures were not present (Mean KLd_{P=0}=0.0038, Mean KLd_{G=0}=0.0054). KLd increased ˜10-fold when protein structure was present (Mean KLd_{P=1}=0.032) and ˜25-fold when glycan structure was present (Mean KLd_{G=1}=0.138). Overall, the presence of glycan structure and the protein structure may provide substantial information about glycan structure.

Next, motif-IMRs were examined by estimating the conditional probability between protein and glycan structures. Conditional probability diverged significantly (Fisher-FDR <0.1) from corresponding marginal probabilities, indicating non-independence. Conditional glycan probabilities (FIG. 3D) and conditional protein structure probabilities (FIG. 3E) show symmetric (Loess estimation) difference from respective marginal probabilities suggesting no global bias for either condition. The glycan-protein structure non-independence-absolute difference between conditional and marginal probabilities—was also investigated, stratified by glycan motif size (number of monosaccharides, FIG. 3F); each motif-size bin contains 279-10,658 IMRs (Table 4). For monomeric-motifs, the change from marginal to conditional probability is 2.3-fold greater when glycan structure is known than when protein structure is known (Mean CMd_{P|G}=0.091, CMd_{G|P}=0.040). As motif size increases, the fold-change between glycan and protein specified CMd grows to 34.2-fold increase at 21-mers (Mean CMd_{G|P}=0.038, CMd_{P|G}=0.10), suggesting larger glycans are less clearly informed by protein structures, but in turn, play a larger role in informing protein structure. Despite being post-translational, glycosylation is known to influence protein folding.

TABLE 4
Distribution and sample size of IMR conditional probabilities

| | | | | N: | N: | | |
|---|---|---|---|---|---|---|---|
| Motif | Structure | | Standard | P(A\|B) > | P(A\|B) < | | Fold |
| Length | Fixed | Mean | Deviation | P(A) | P(A) | N | Change |
| 1 | glycan | 0.039501 | 0.065099 | 548 | 634 | 1183 | 2.314301 |
| 1 | protein | 0.091417 | 0.13807 | 548 | 634 | 1183 | |
| 2 | glycan | 0.033059 | 0.058625 | 1025 | 1160 | 2185 | 2.519216 |
| 2 | protein | 0.083284 | 0.117493 | 1025 | 1160 | 2185 | |
| 3 | glycan | 0.027672 | 0.054289 | 1576 | 1788 | 3364 | 3.235929 |
| 3 | protein | 0.089546 | 0.115724 | 1576 | 1788 | 3364 | |
| 4 | glycan | 0.025251 | 0.045312 | 1798 | 2056 | 3855 | 3.704429 |
| 4 | protein | 0.093541 | 0.120251 | 1798 | 2056 | 3855 | |
| 5 | glycan | 0.025405 | 0.04588 | 2327 | 2628 | 4956 | 3.470337 |
| 5 | protein | 0.088163 | 0.104168 | 2327 | 2628 | 4956 | |
| 6 | glycan | 0.024508 | 0.047592 | 2941 | 3369 | 6311 | 3.562296 |
| 6 | protein | 0.087305 | 0.098485 | 2941 | 3369 | 6311 | |
| 7 | glycan | 0.023376 | 0.045586 | 4131 | 4761 | 8893 | 4.097588 |
| 7 | protein | 0.095787 | 0.103835 | 4131 | 4761 | 8893 | |
| 8 | glycan | 0.022285 | 0.041808 | 4898 | 5711 | 10612 | 4.714163 |
| 8 | protein | 0.105054 | 0.109607 | 4898 | 5711 | 10612 | |
| 9 | glycan | 0.021484 | 0.037508 | 4913 | 5742 | 10658 | 5.043449 |
| 9 | protein | 0.108351 | 0.11061 | 4913 | 5742 | 10658 | |
| 10 | glycan | 0.020017 | 0.032146 | 4319 | 5183 | 9506 | 5.641682 |
| 10 | protein | 0.11293 | 0.113366 | 4319 | 5183 | 9506 | |
| 11 | glycan | 0.019036 | 0.026794 | 3525 | 4367 | 7895 | 5.758148 |
| 11 | protein | 0.109612 | 0.107277 | 3525 | 4367 | 7895 | |
| 12 | glycan | 0.017643 | 0.022947 | 2579 | 3304 | 5885 | 5.907276 |
| 12 | protein | 0.104222 | 0.096894 | 2579 | 3304 | 5885 | |
| 13 | glycan | 0.015433 | 0.018858 | 1894 | 2475 | 4371 | 6.921746 |
| 13 | protein | 0.106823 | 0.096742 | 1894 | 2475 | 4371 | |
| 14 | glycan | 0.012897 | 0.014746 | 1399 | 1856 | 3255 | 8.261073 |
| 14 | protein | 0.106543 | 0.094723 | 1399 | 1856 | 3255 | |
| 15 | glycan | 0.010178 | 0.011166 | 931 | 1253 | 2185 | 11.10655 |
| 15 | protein | 0.113045 | 0.097098 | 931 | 1253 | 2185 | |
| 16 | glycan | 0.008308 | 0.008946 | 623 | 835 | 1458 | 14.74224 |
| 16 | protein | 0.122482 | 0.102175 | 623 | 835 | 1458 | |
| 17 | glycan | 0.006909 | 0.007713 | 427 | 565 | 993 | 19.85786 |
| 17 | protein | 0.137195 | 0.108164 | 427 | 565 | 993 | |
| 18 | glycan | 0.006178 | 0.006757 | 275 | 359 | 634 | 22.56627 |
| 18 | protein | 0.139421 | 0.107794 | 275 | 359 | 634 | |
| 19 | glycan | 0.004446 | 0.004865 | 158 | 214 | 372 | 31.24682 |
| 19 | protein | 0.13892 | 0.107965 | 158 | 214 | 372 | |
| 20 | glycan | 0.004446 | 0.004865 | 158 | 214 | 372 | 31.24682 |
| 20 | protein | 0.13892 | 0.107965 | 158 | 214 | 372 | |
| 21 | glycan | 0.00363 | 0.003766 | 118 | 161 | 279 | 34.24255 |

| | | | | | | |
|---|---|---|---|---|---|---|
| 21 | protein | 0.124301 | 0.101235 | 118 | 161 | 279 |

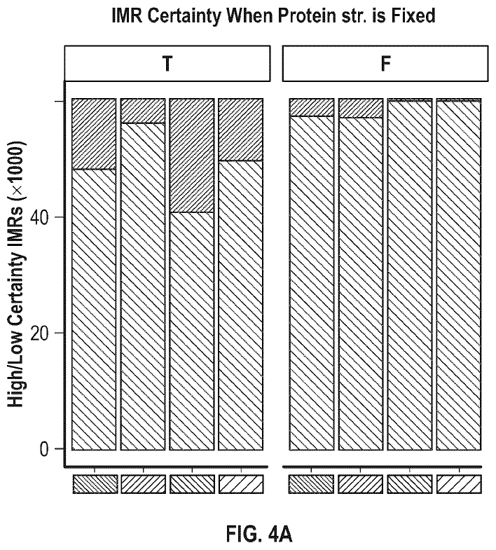

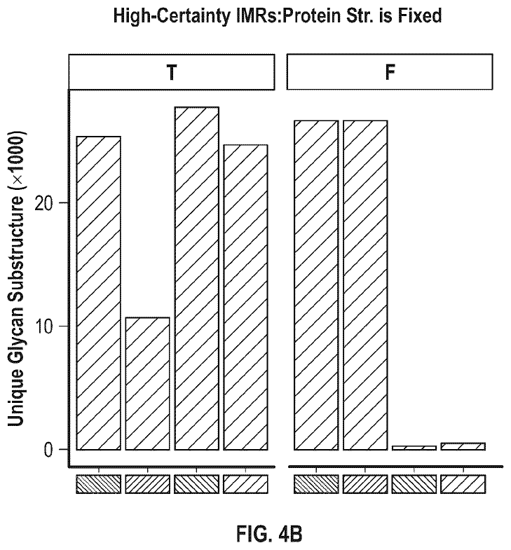

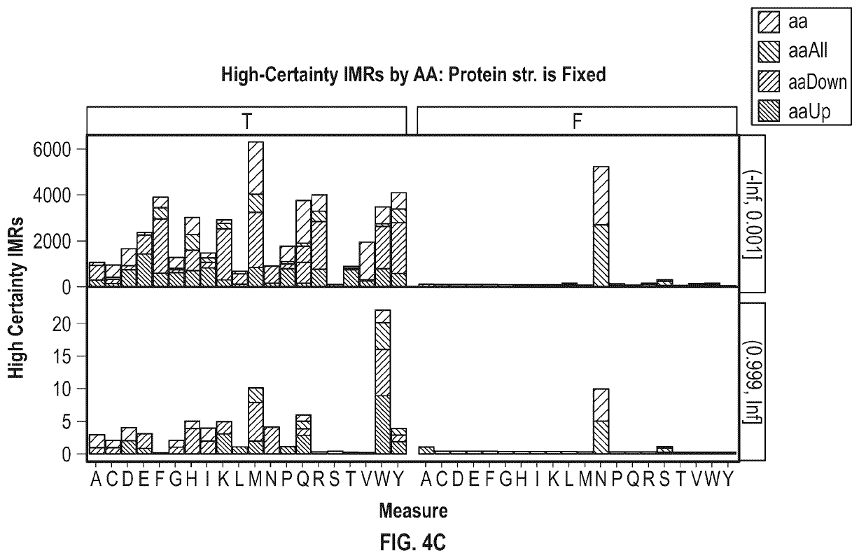

Many AA-proximal IMRs are high-confidence, wherein Pr(GP) is close to 1 or 0 (within 0.001). Confidence in glycan presence increases when a specific protein structure feature is present (FIG. 4A). Of IMRs involving a spatially proximal AA, 20.2% are highly deterministic of glycan substructures. Additionally, 32.4% and 17.5% of down- and upstream AA IMRs (+/−6aa) are highly deterministic. The certainty with which an AA determines a glycan structure decreases substantially when proximal AAs are absent to 5%, 0.3% and 0.33% for spatially, downstream and upstream-proximal residues respectively (FIG. 4A). These high-confidence IMRs do not appear to be dominated by small numbers of motifs as the high-confidence IMR count is proportional to the number of unique substructures (FIG. 4B).

Among the highly deterministic protein-glycan relations, (among 1725 N-glycosylation events) 1553 glycans were observed to contain a GlcNAc on the β-1,6-mannose branch (Glc2NAc(β1-6)Man(α1-6)Man(β1-4)Glc2NAc); indicative of a hybrid or complex N-glycan. All 75 glycosylation events with a downstream tryptophan included glycans with the hybrid/complex substructure (Table 4). These data suggest tryptophan may be sufficient to result in a decrease of oligomannosylation. Similarly, in 454 O-glycosylation events, 237 contain the sialyl-T antigen (Neu5Ac(α2-6)Gal(β1-3)GalNAc). Of 6 events containing a sequence-proximal tryptophan, every event also contained a sialyl-T antigen (Table 4).

The mapping between glycan and protein structures was described quantitatively. The specific IMRs were quantified using univariate logistic generalized estimation equations (GEE) to probe the site-matched glycan-protein co-occurrences in PGES-DB and control for protein identity effects as described elsewhere herein. The resulting odds ratios (OR) estimate the probability a glycan substructure will appear given a proximal protein structure-feature. Therefore, a list of ORs-protein-glycan structure co-occurrence likelihood—for each glycan substructure association with one protein structure-feature describes the typical glycosylation observed close to a given protein structure; “expected substructure abundance” or an “expected glycoprofile” for that protein structure. Therefore, when expected glycoprofiles for all glycan substructures is compared across protein structure-features, e.g., the expected glycoprofile change between alanine and isoleucine, glycosylation impact, or “glycoimpact,” of AA-substitution may be estimated by considering the difference in expected glycoprofiles for across all glycan substructures.

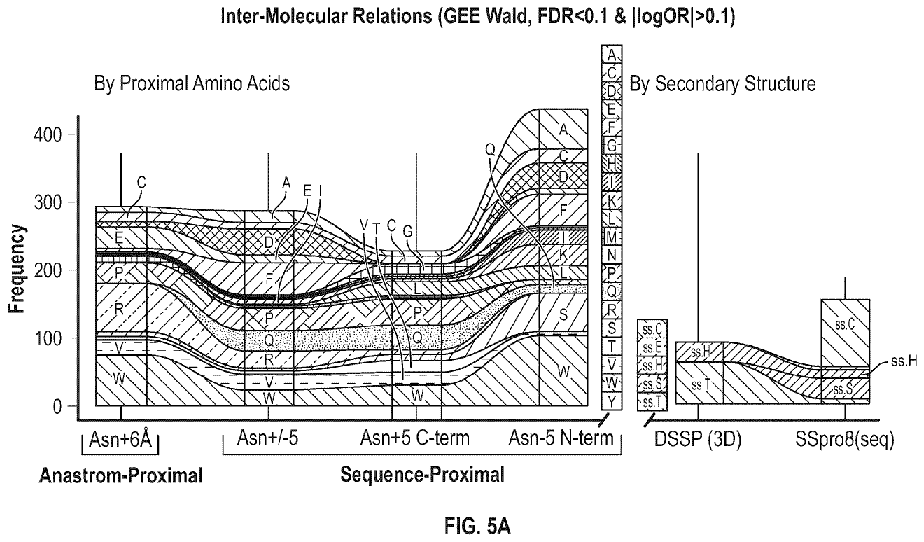

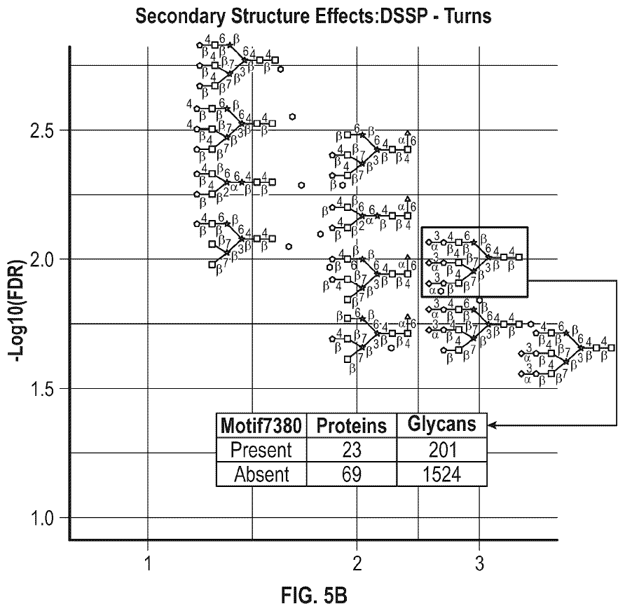

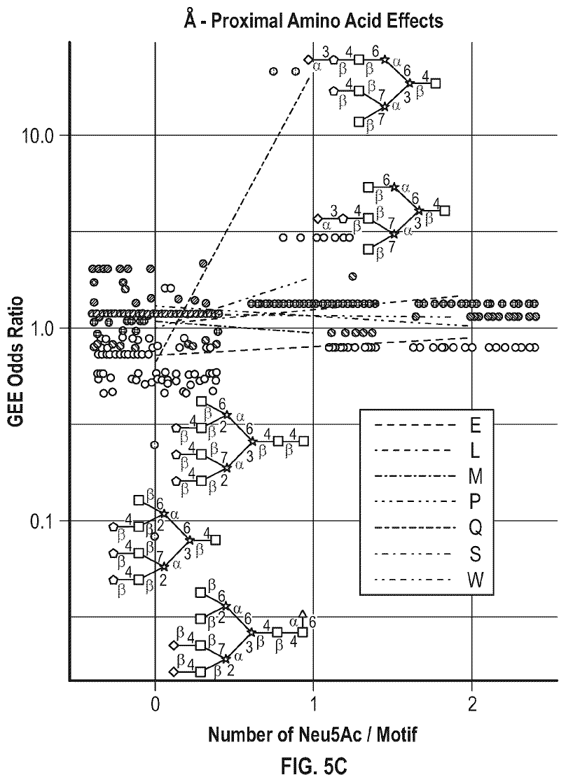

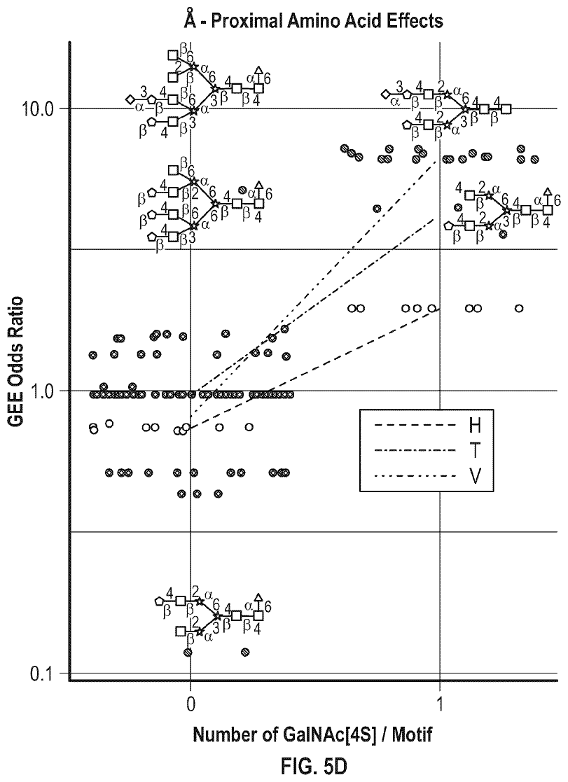

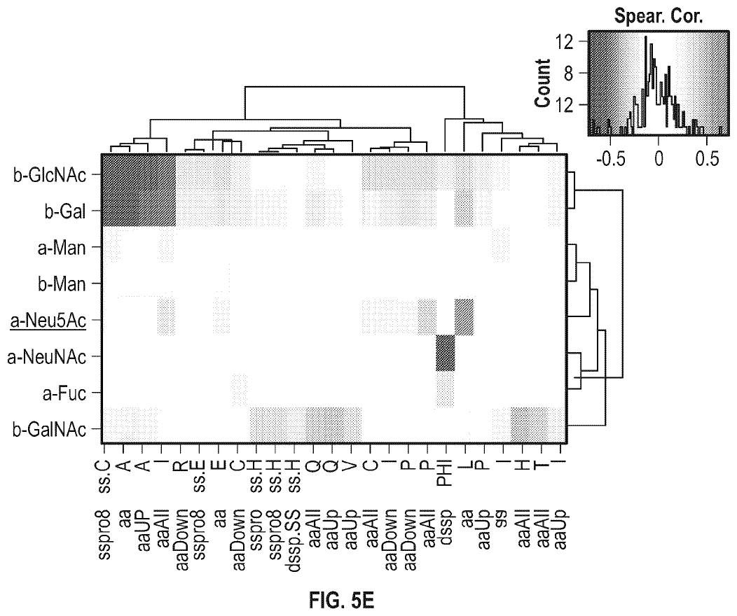

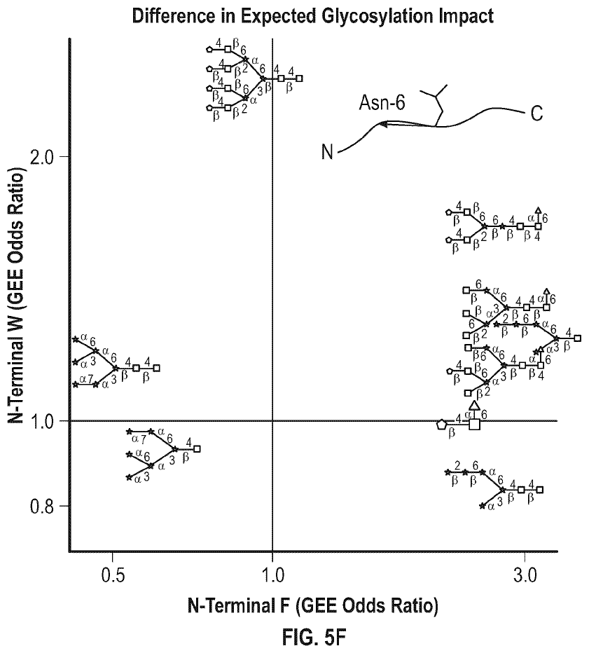

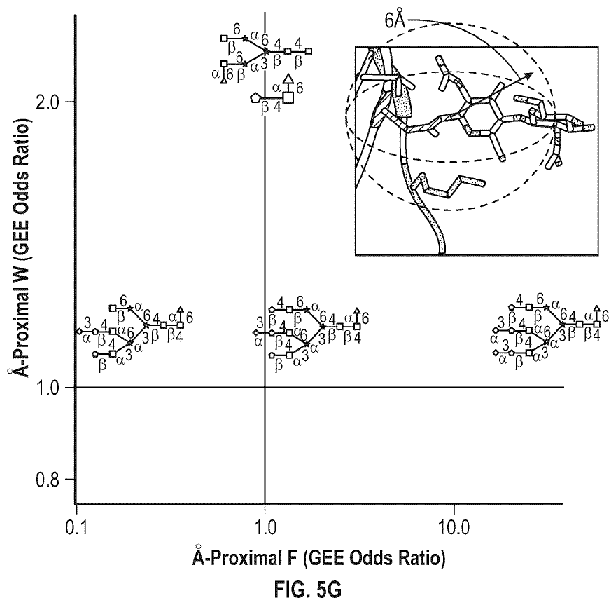

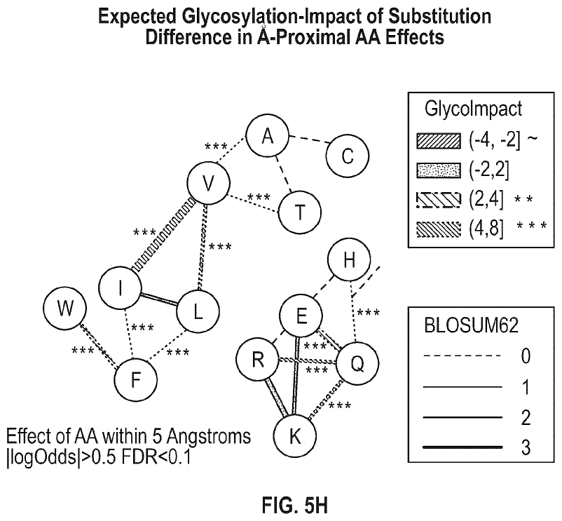

1,715 (FDR<0.1, |log(OR)|>0.1) N-glycan IMRs were discovered. Many IMRs associated with structure-proximal (e.g., N+6 Å) and sequence-proximal (e.g., N+/−5 residues) AAs were found. Stratifying sequence-proximal effects, approximately twice as many IMRs involving upstream (N-5) than downstream AAs were observed. Among the downstream AA effects, tryptophan, alanine, serine and phenylalanine are most impactful (99, 55, 55, and 48 IMRs respectively). Tryptophan also has many IMRs when downstream and physically proximal (26). Arginine and glutamine are the largest effectors when structurally proximal (70) or downstream (35). Finally, glycosylation sites on turns have the most IMRs (61) (FIG. 5A).

Turn-associated IMRs include >3-fold increases in di- and tri-sialylated tetra-antennary and >2-fold increases in mono- and di-galactosylated structures with core fucose; all positively correlated structures have at least one galactose while not all are core (FIG. 5B). Structurally proximal glutamine is associated with a >20-fold increase in monosialylated triantennary structures and a 10-fold decrease in tetra-antennary structures (FIG. 5C). Histidine, threonine and valine show increasing correlation with GalNAc[4S] (FIG. 5D). Spearman correlation biclustering between the number of monosaccharides per substructure and protein structure-features in an IMR suggest there may be two major types of protein structure influence mirroring the well-known N-glycan/O-glycan dichotomy in glycosylation. Providing clues to the elusive O-glycosylation site, proximal alanine is negatively correlated with galactose and GlcNAc but positively correlated with GalNAc. Conversely, threonine and histidine are positively associated with GIcNAc and Galactose but negatively correlated with β-GalNAc. GlcNAc and Gal-complex-glycan substituents-follow similar trends to Neu5Ac. However, Neu5Ac, GlcNAc and Gal trends diverge near proline, cysteine and valine; suggesting these AAs may be limiters of high-complexity (FIG. 3E).

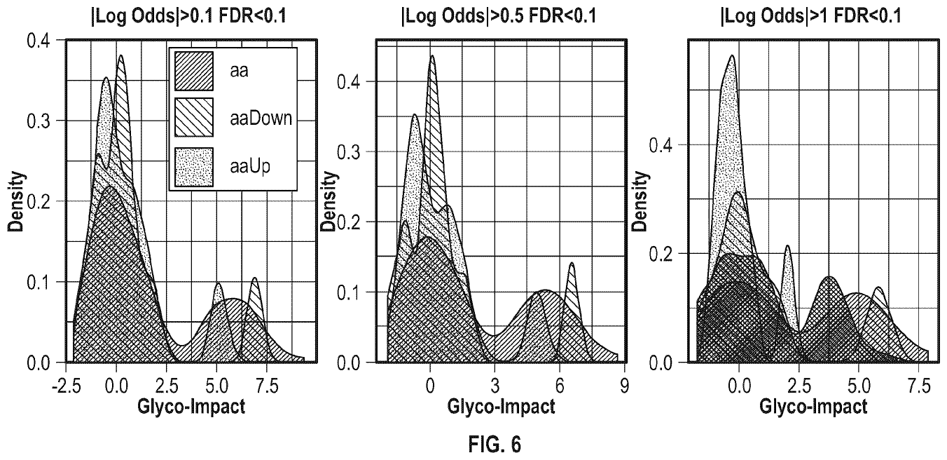

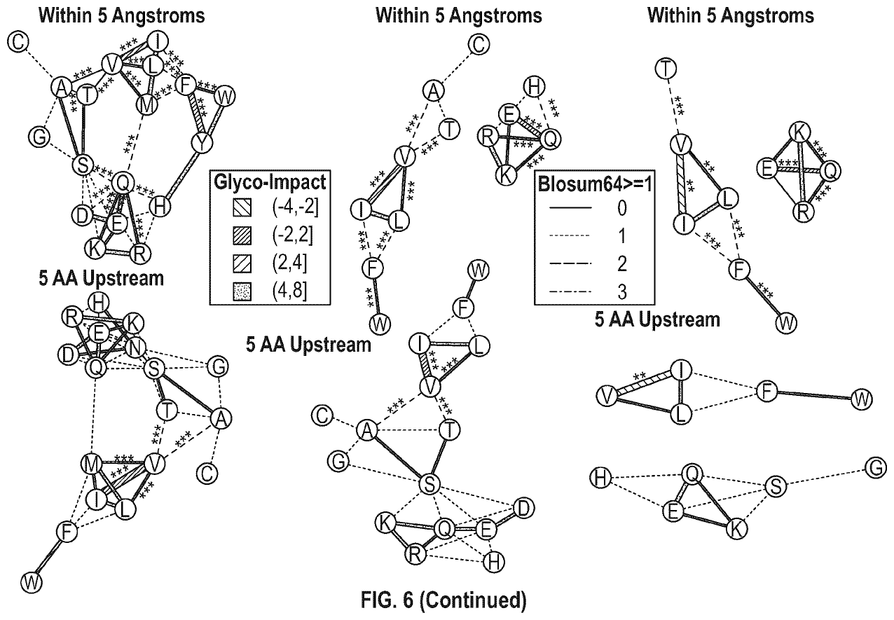

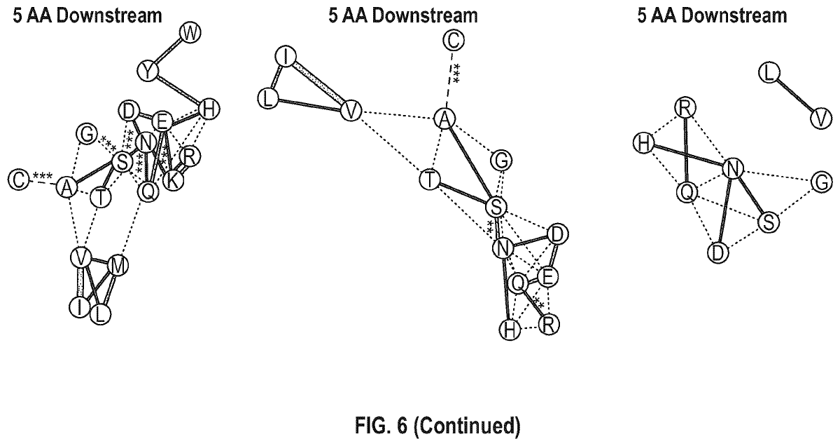

Given the expected glycoprofile for each protein-feature, the “glycoimpact” of a variety of protein structural transformations (e.g. AA-substitutions) may be explored; the impact of that transformation on glycan biosynthesis. More specifically, glycoimpact may be defined as the difference between two expected glycoprofiles; the expected difference across two protein structure-features resulting from transformation between those protein structure-features. For example, the relative impact of phenylalanine and tryptophan, two structurally similar aromatics, were compared. An upstream phenylalanine was associated (>3-fold) with core fucosylated tri- and biantennary structures with variable galactosylation. Tryptophan was marginally associated (<2-fold) with core-fucosylated biantennary structures too but more associated (>2-fold) with tetra-antennary structure, suggesting a phenylalanine/tryptophan substitution could impact branch number and core-fucosylation. Upstream phenylalanine was associated (>3-fold) with a Man7 substructure but anti-correlated with a Man6 substructure (>2-fold) suggesting that upstream phenylalanine, in some contexts, prefers larger oligomannosidic structures (FIG. 5F). At the structural-level, proximal phenylalanine and tryptophan show related effects. Structure-proximal phenylalanine was correlated (>10-fold) with an increase in sialylation on tri-antenary core-fucosylated structures while Trp was correlated with distal fucosylation (>2-fold) (FIG. 5G). Measured as the normalized Euclidean distance between tryptophan and phenylalanine expected glycoprofiles, the tryptophan/phenylalanine substitution is predicted to be highly glycoimpactful (>4-fold). All AA-substitution glycoimpact scores were calculated as the difference in expected glycoprofiles between each AA-pair. Glycoimpact was calculated at multiple IMR thresholds (FIG. 6). Representative substitution events are shown in FIG. 5H. The glycoimpact AA-substitution matrix may be referred to herein as the BLOSUM-PAM Orthology matrix (BLAMO X:Y); X and Y refer to the log OR and FDR thresholds respectively. Log ORs insignificant or unsubstantial by the X:Y threshold are excluded from the glycoimpact calculation. For example, FIG. 5H, displays a subset of BLAMO 0.5 0.1 relations.

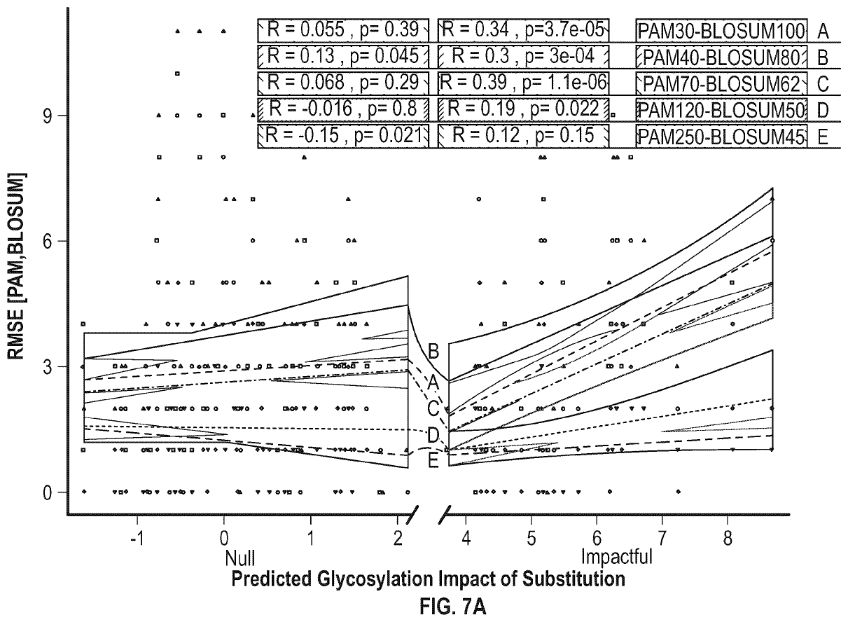

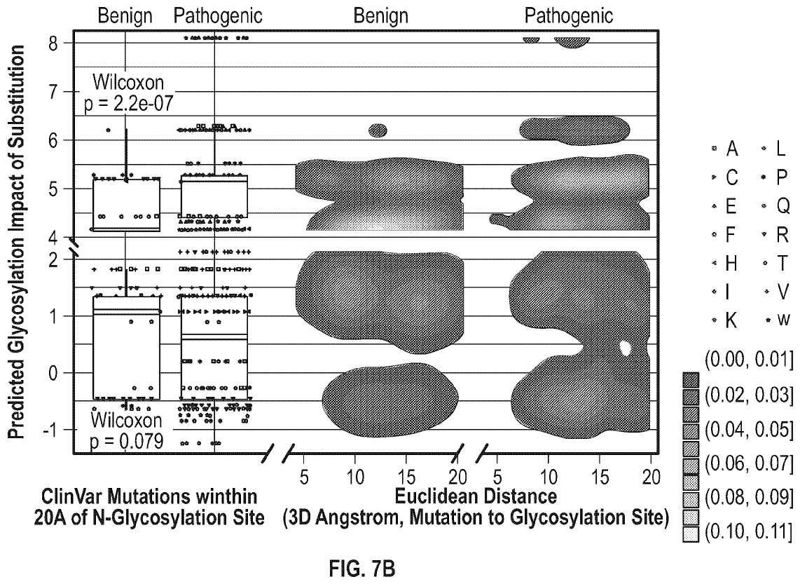

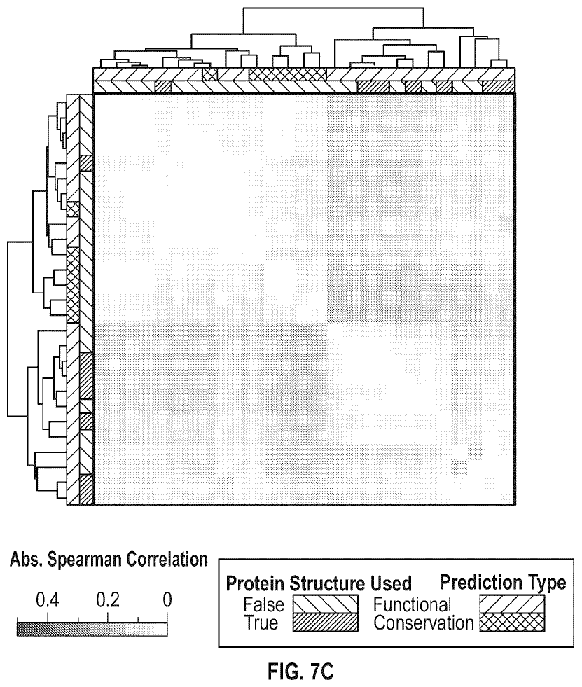

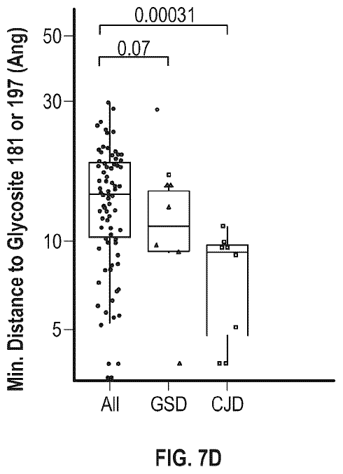

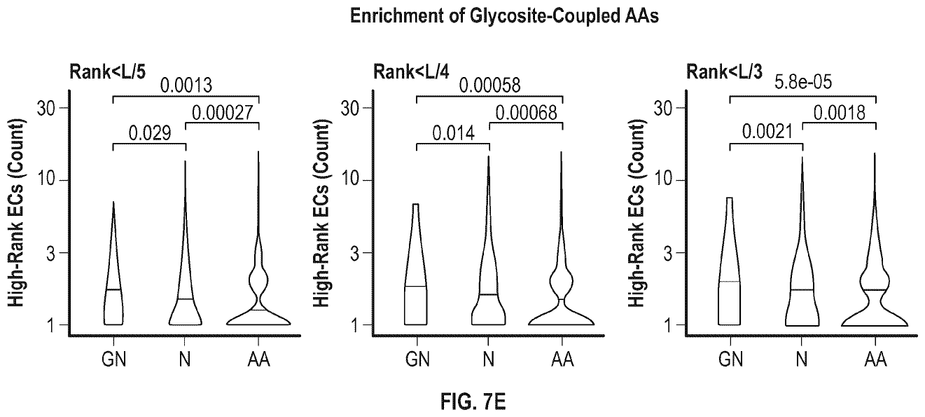

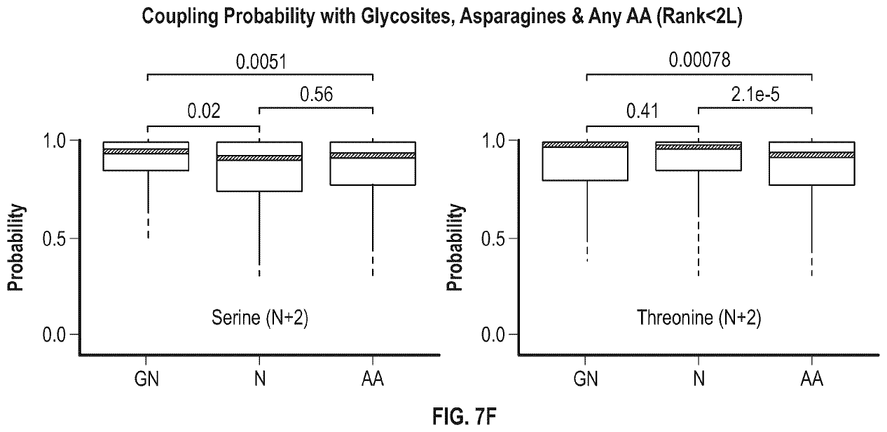

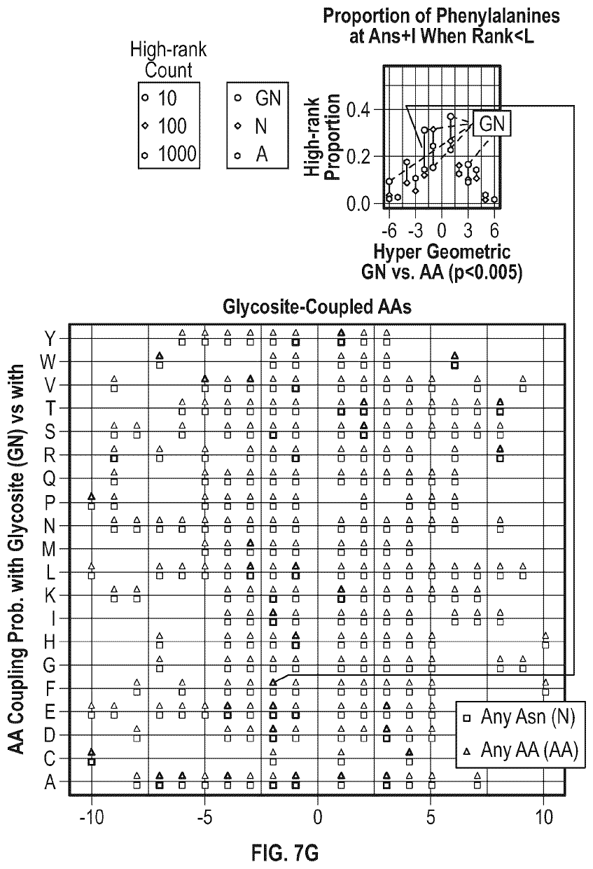

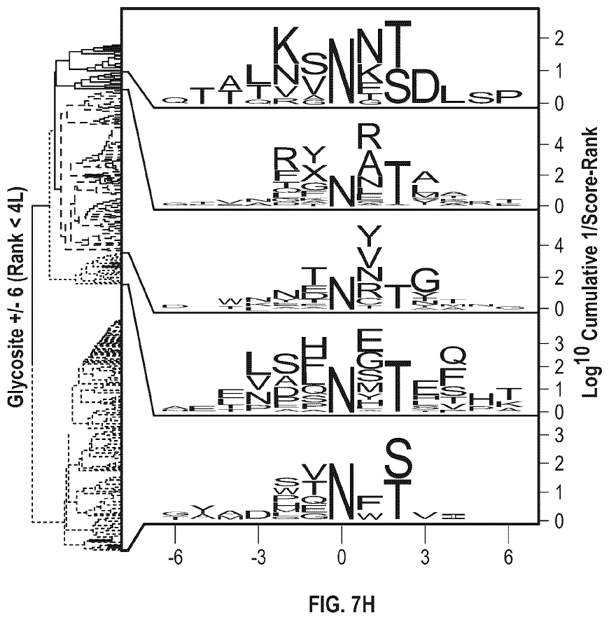

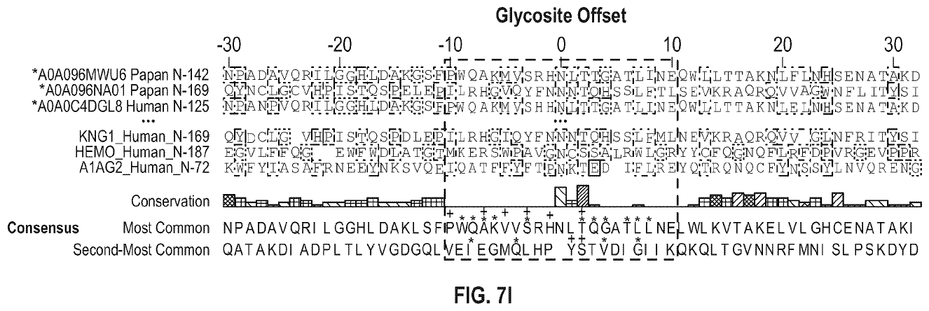

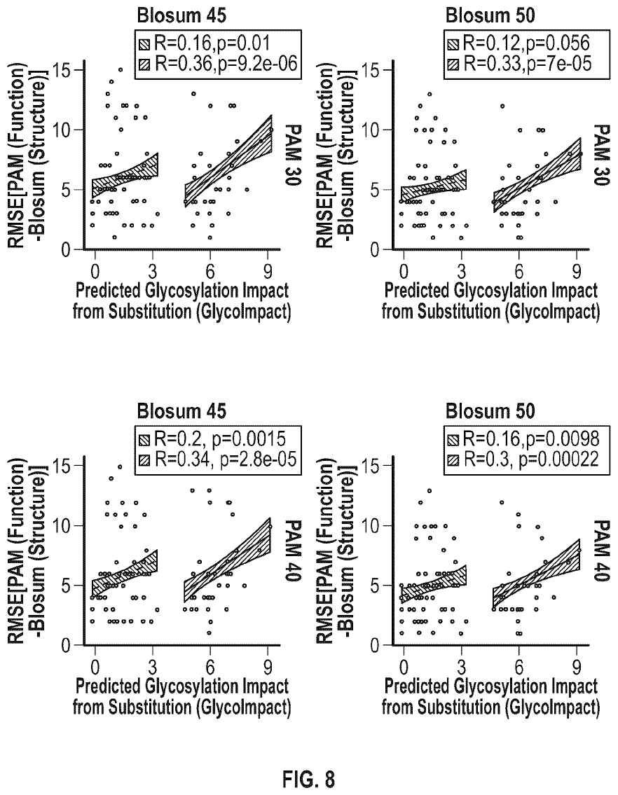

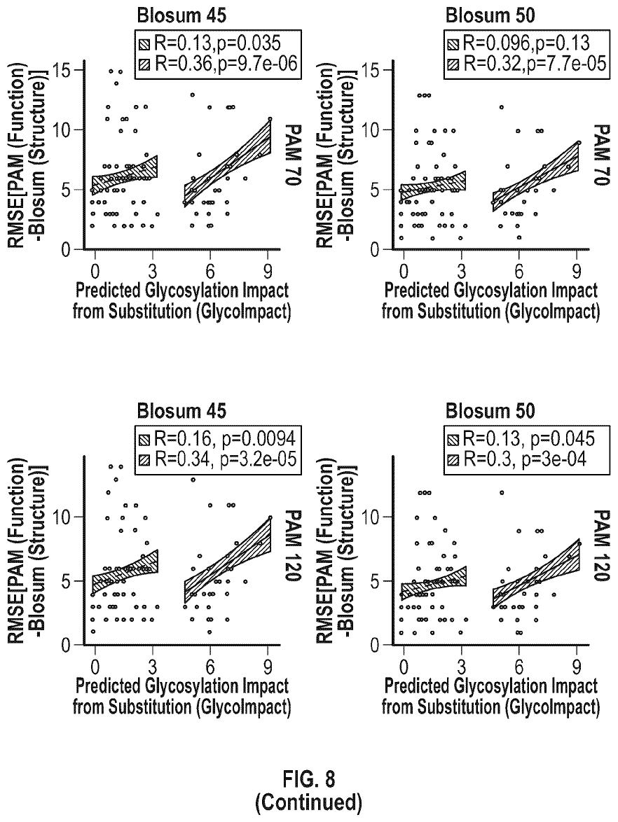

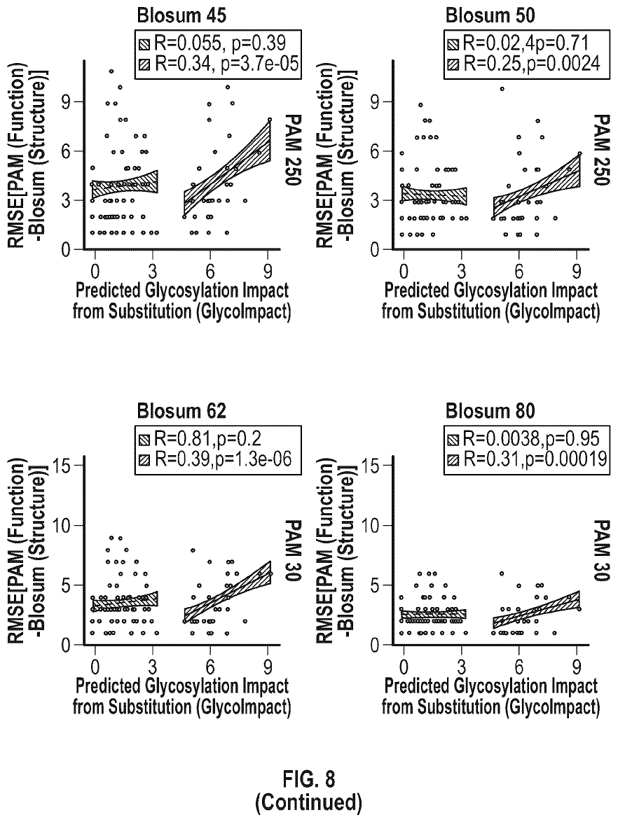

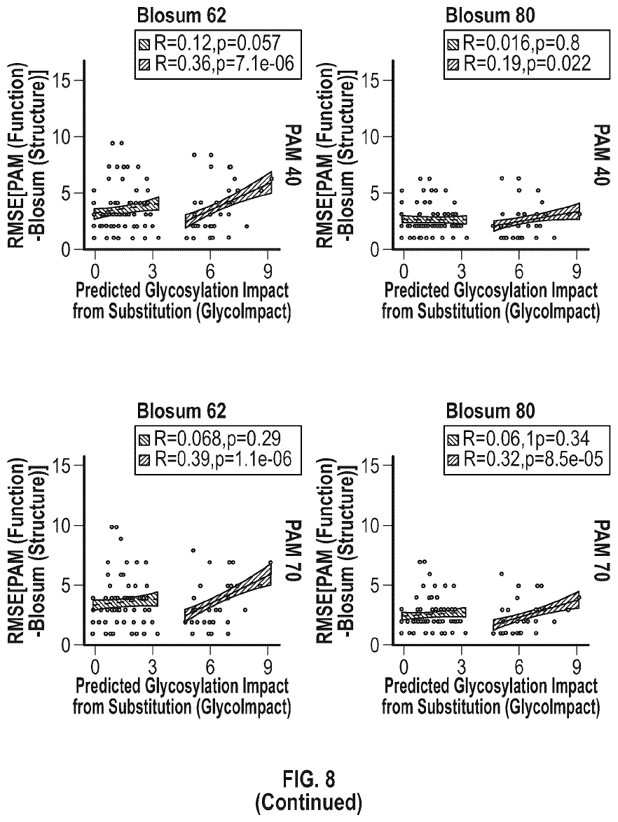

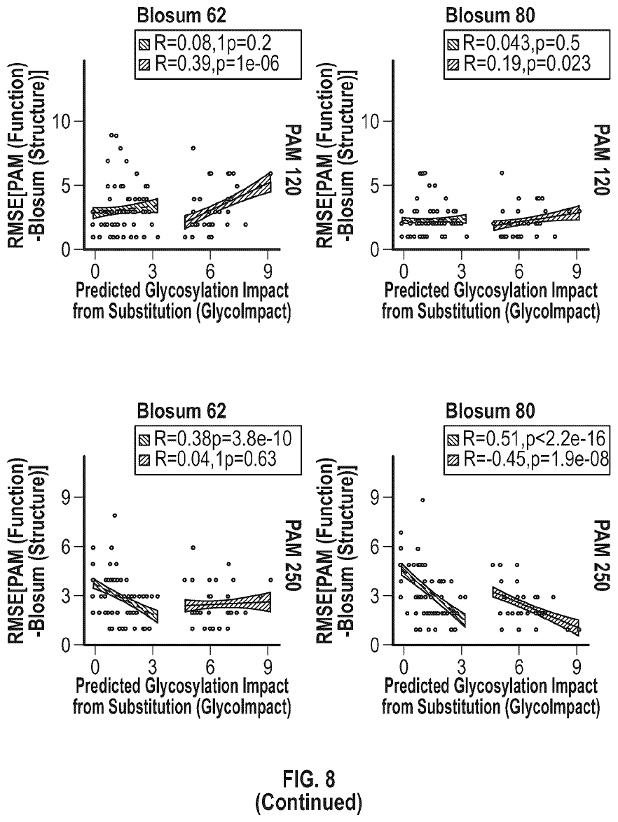

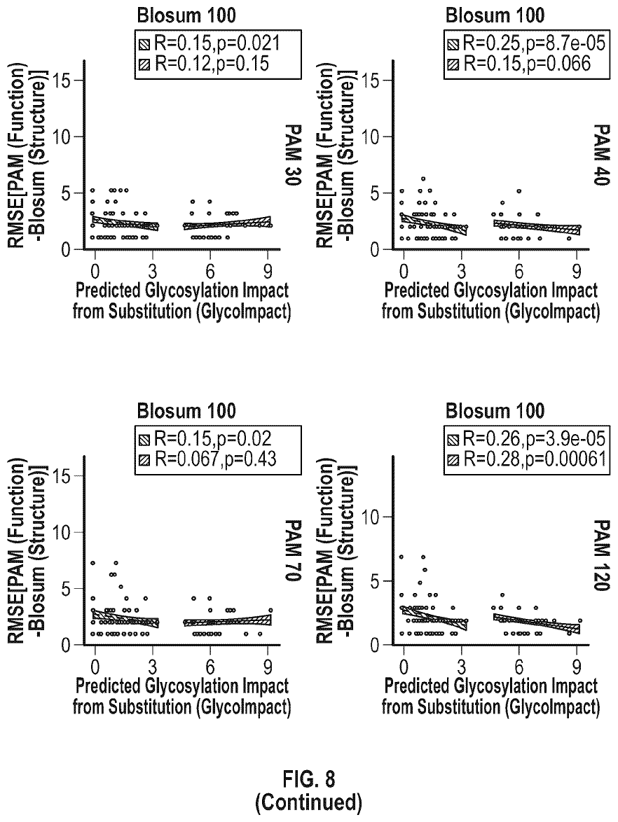

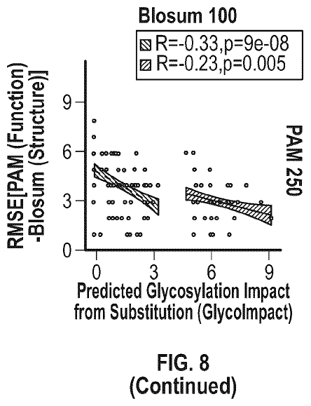

To further establish the relevance of glycoimpact, it was compared to established measures of amino acid substitution impact. The PAM and BLOSUM matrices are popular but distinct amino acid substitution matrices. PAM is based on global alignments within a protein focusing it on evolution and function. Meanwhile BLOSUM uses local alignment across proteins to highlight structure and conserved domains. Glycoimpact (BLAMO 0.5:0.1) was compared to the divergence between the function-focused PAM and structure-focused BLOSUM matrices. Comparing PAM and BLOSUM scores at multiple thresholds (RMSE(PAM_{i,j},BLOSUM_{i,j}), FIG. 7A, FIG. 6), error in 4 of 5 PAM-BLOSUM comparisons was found to be significantly correlated to glycoimpact for impactful (z>2.5) substitutions; correlation diminished for null-glycoimpact substitutions (FIG. 7A, FIG. 6). The correlation between high-glycoimpact substitutions and PAM-BLOSUM error was maintained for most PAM and BLOSUM thresholds (FIG. 8). These results suggest a positive relationship between glycoimpact and the failure of structure (BLOSUM) to explain function (PAM). Given this relationship, the glycoimpact substitution predictions may be referred to herein as the BLOSUM-PAM Orthology matrix or “BLAMO.”

Glycoimpact as a measure of pathogenicity of ClinVar mutations within 20 Å (3D min-distance; minimum distance between any two atoms) of a glycosite annotated in UniprotKB was examined. Null and impactful glycoimpact (BLAMO0.5:0.1) were examined, and glycoimpact was found to be significantly higher (Wilcoxon p=2.2e−7) for ClinVar-pathogenic mutations close to glycosylation sites. The difference trends towards inversion for null glycoimpact values (Wilcoxon p=0.079) (FIG. 7B). One example high-glycoimpact and glycosite-proximal mutation is tvrosinase/A355V (P14679), a glycosylation-associated causal mutation in albinism.

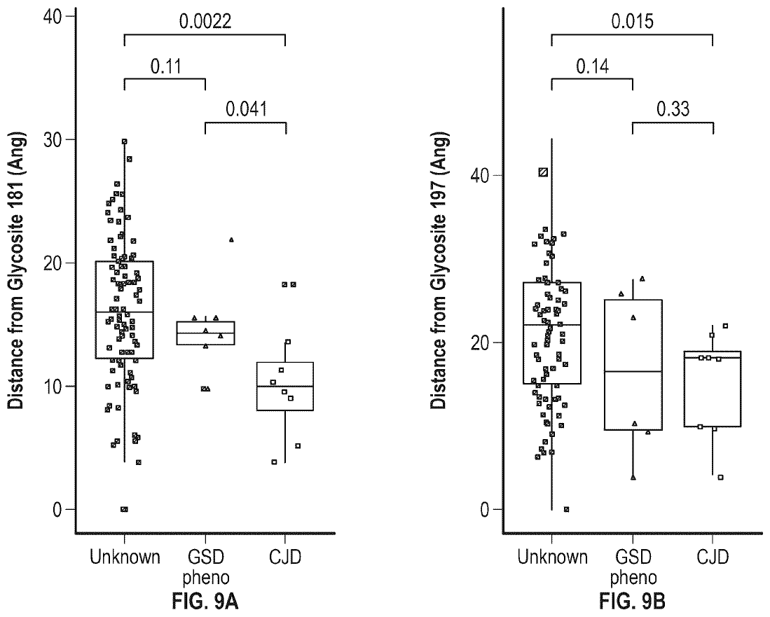

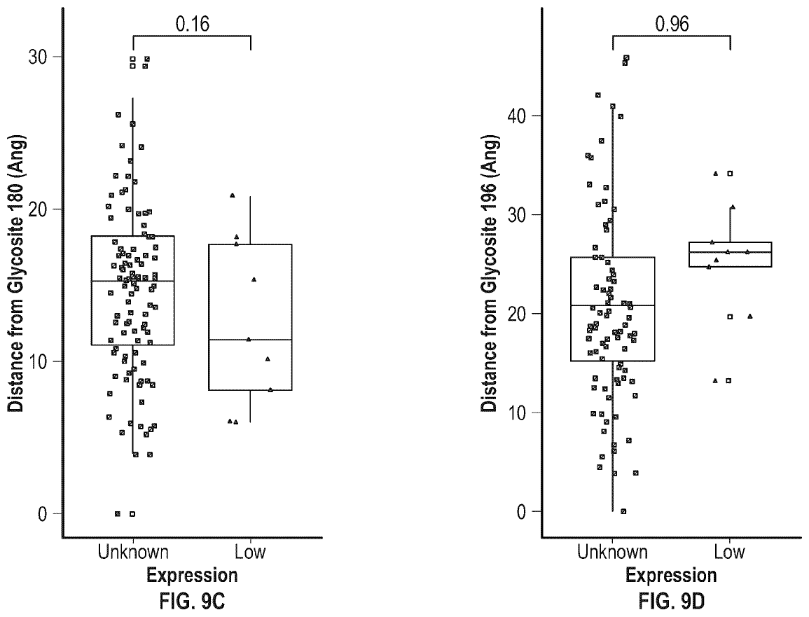

To determine the proximity of prion disease causing mutations to glycosites, the 3D min-distance from all positions in human PrP (including mutations causing Creutzfeldt-Jakob disease (CJD) and Gerstmann-Straussler disease (GSD)) to the two PrP glycosylation sites, N181 and N197, were measured (FIG. 7D). CJD-causing mutations were approximately twice as close to glycosylation sites than the background distribution of all PrP sites (One-sided Wilcoxon p=0.0003). GSD-causative mutants were also found to trend closer (One-sided Wilcoxon p=0.07) (FIG. 7D, FIG. 9A-9B). Low expression mutants, an indication of possible aberrant glycosylation, were found to trend closer to site N180 (One-sided Wilcoxon p=0.16) and appeared further from N196 (One-sided Wilcoxon p=0.04) (FIG. 9C-9D). Thus, searching for glycan-modulated pathogenic events may be possible using their glycosylation sites or known mutations as a reference point.

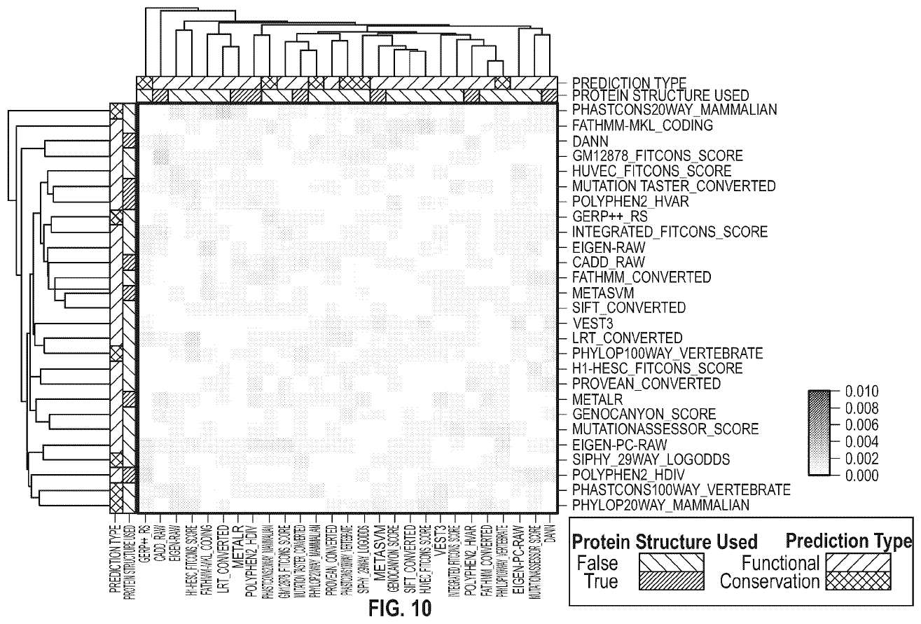

To test if differences in prediction scores across variants could be explained by glycoimpact (BLAMO 0.5 0.1) of their corresponding amino acid changes (see Methods), across 3,549,910 nonsynonymous mutations, the disagreement (RMSE) between each of 27 rank-normalized functional impact prediction tools (precomputed with dbNSFP) was measured; impact prediction divergence was correlated with glycoimpact. After hierarchical clustering on the correlation coefficients, tools were separated into two main clusters: one that primarily contained conservation and sequence and/or epigenetic-based tools and another that contained nearly all (6/7) of the protein-structure based tools (FIG. 7C). Nearly all variant impact score differences across the two clusters demonstrated marginally significant correlations with glycodistance. However, these correlations and clustering structure disappear when glycoimpact scores are shuffled (FIG. 10). Ablation by shuffling suggest that glycoimpact explain functional discrepancies between prediction scores.

To explore evolutionary pressures acting near the glycosite, evolutionary coupling (EC) scores (i.e., the likelihood that amino acids will co-occur in a protein) were calculated from functional-domain alignments of 2,005 glycoproteins. Coupling scores for top-ranked amino acid pairs were examined; using multiple score cutoffs from L/5 to 4L (L is the protein alignment length). The number of high-ranking ECs between any amino acid with N-glycosylation sites (GN) was examined. At multiple thresholds, significantly more high-ranking glycosite-coupled ECs (GN) were found than Asn-coupled (N, p<0.025, one-sided Wilcoxon-test) or all background ECs (X, p<0.0013, one-sided Wilcoxon-test, FIG. 7E). GN, N, and X couplings were compared with specific amino acids i-positions N-terminal (N-i) or C-terminal (N+i). Glycosite-coupling with another position-specific amino acid as an increased GN-coupling probability relative to N or X at a given rank threshold (one-sided Wilcoxon test, FIG. 7F) or as increased proportion of high-ranking NG-coupled events relative to N or X (FIG. 7G) was tested; increased proportion was measured by hypergeometric enrichment multiple rank thresholds then pooled using Fisher's method then corrected for multiple-testing. Serine and Threonine were found to be significantly more coupled with glycosylation sites at the N+2 position. The glycosite-coupling enrichment with Serine and Threonine was significant as measured by the relative distributions of coupling probabilities (One-sided Wilcoxon p<0.05, FIG. 7F) and the relative number of high-ranking couplings at multiple thresholds (pooled hypergeometric FDR<0.1, FIG. 7H). Several additional position-specific glycosite-coupled residues were found, including phenylalanine at N-2 (hypergeometric p<0.005, FIG. 7G; pooled hypergeometric FDR <0.1, FIG. 7H), Tyrosine at N-1 (pooled hypergeometric FDR <0.1, FIG. 7H), and Tryptophan at N-2 and N-3 (hypergeometric p<0.2 at multiple rank-thresholds).

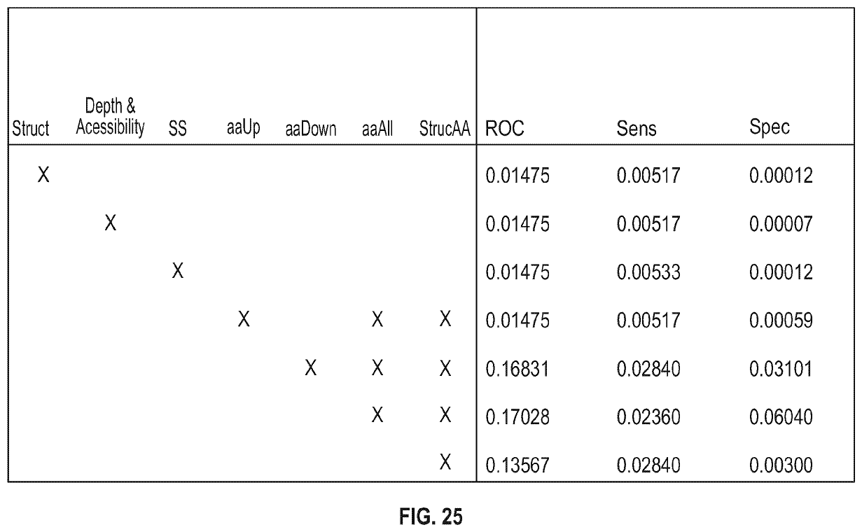

Examining position-specific glycosite couplings (hypergeometric enrichment for high-rank ECs pooled across rank-thresholds, FIG. 7G-7H), 13 of 20 amino acids were found to have at least one significant (FDR<0.1) increase in co-occurrence with glycosites over other asparagines (N, red-square) or any amino acid (X, black triangle); position-specific glycosite coupling events enriched over N and X may expand the definition of the sequon while those with either N or X enrichment may be more indicative of glycosite emergence. Seven of ten amino-acids implicated in upstream glycosite interactions (those visible in FIG. 2A) show enriched coupling with glycosites; specifically alanine (N-1,2,4,6), aspartic acid (N-2), phenylalanine (N-2), isoleucine (N-2), lysine (N-2), leucine (N-1,3,5), and serine (N-2). Several glycosite-coupling events were enriched over either N or X but not both. When coupling probability rank was pooled for each glycosite-relative position, evidence of larger co-coupling events was found. Sequons (e.g., N+/−6) were masked by EC score (only rank<4L were retained), then motifs were clustered and motifs were constructed for 5 motif-clusters (FIG. 7I) and 25 motif-clusters. The N+2 aspartic acid enriched in the univariate analysis (FIG. 7H) co-occurs with an N-2 Lysine (FIG. 7I, motif 1). Alternatively, glutamic acid was found more likely to co-occur with other glutamic acid residues (N-4,+1,+3) with an N+2 threonine sequon (FIG. 7I, motif 4). These couplings, reflective of evolutionary pressures, surrounding the glycosylation sites suggest a dramatic expansion of the N-glycosylation site structure.

Finally, a glycosite-centered alignment of glycosites permitting a tetra-antennary N-glycan with no fucose or sialic acids was examined (FIG. 7J). The glycosite alignment was examined for consistency with high-influence amino acids (Of 20 amino acids, 10 upstream residues, and 8 downstream residues, FIG. 5A) and those significantly coupled with glycosylation sites (1.9 residues per position, FIG. 7H). Sixteen of 20 glycosite-flanking amino acids show consistency between the first or second most common amino acids and either the high-influence or glycosite coupled residues. In the primary glycosite consensus sequence (PWQAKVVSRHNLTQGATLLNE (SEQ ID NO: 64), N+/−10, FIG. 7J), 5 high-influence residues appearing upstream (S, K, A, Q, W; binomial N=10, p=10/20, Pr(X>5)=0.377) and the 6 high-influence amino acids appearing downstream (T, Q, G, T, L, L; binomial N=9, p=8/20, Pr(X>6)=0.025) indicated an enrichment of highly glyco-influential downstream residues in the consensus. Glycosite-coupled residues in the primary consensus sequence were enriched upstream in the glycosite alignment (P, A, V, S, H; binomial N=10, p=1.9/10, Pr(X>5)=0.00488). At nearly every glycosite-flanking residue (N+/−10), indications of consistency were seen between these three analyses.

Finally, to validate the specificity and portability of the predictions, PGES-DB calculated IMRs were compared to well-studied glycosylation on specific glycoproteins.

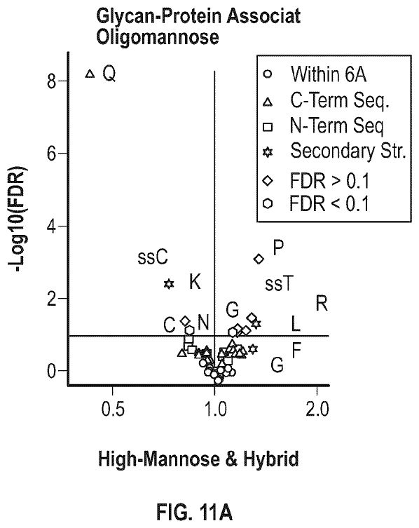

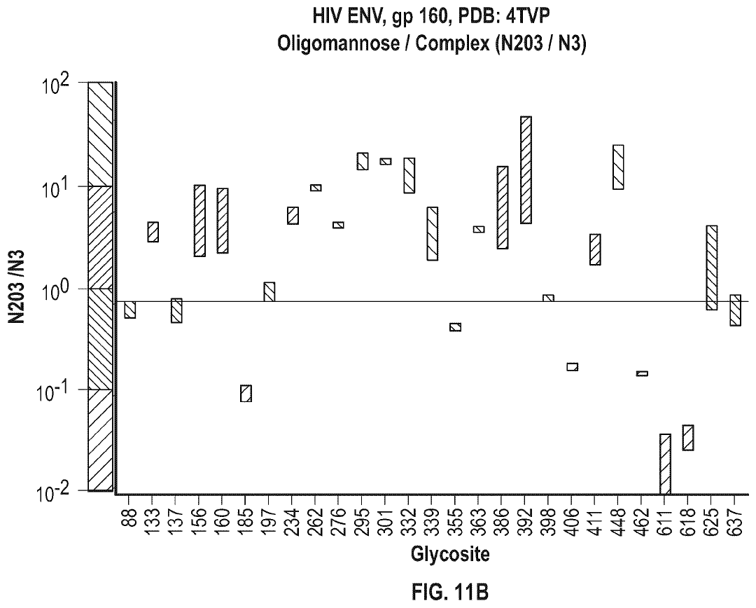

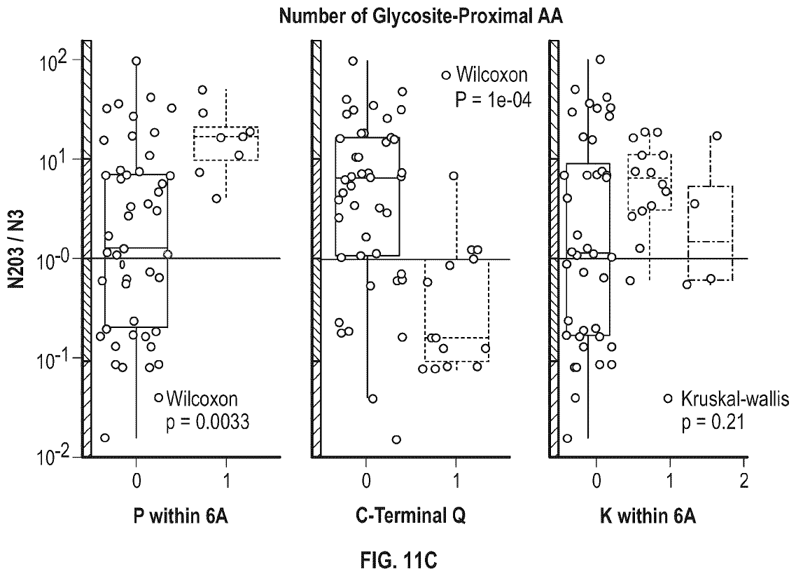

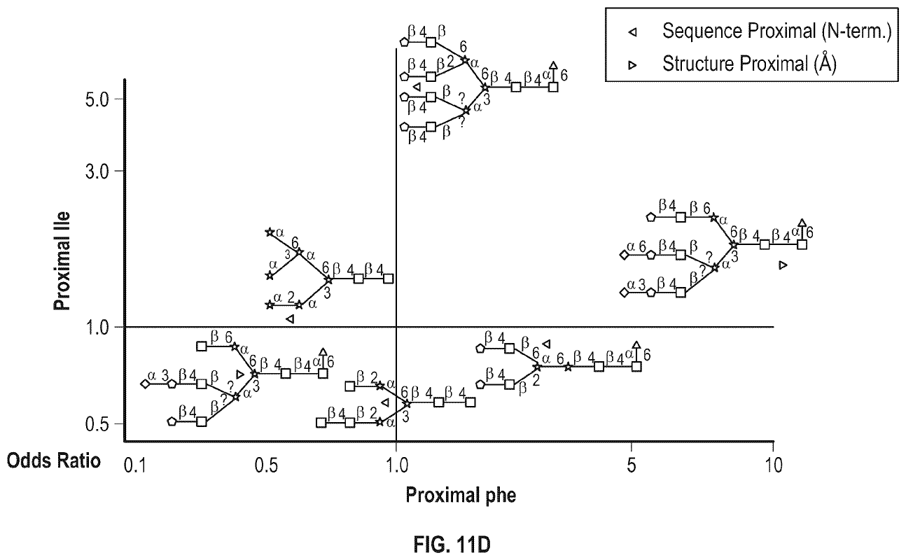

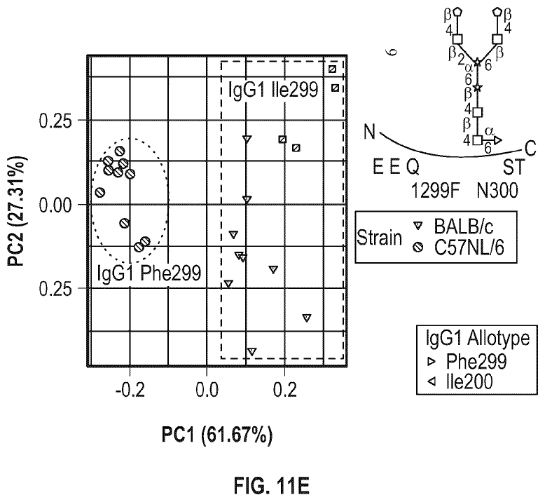

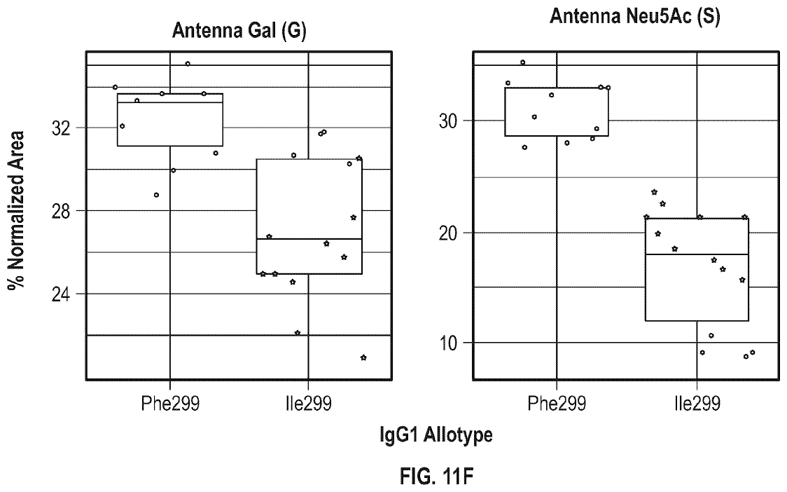

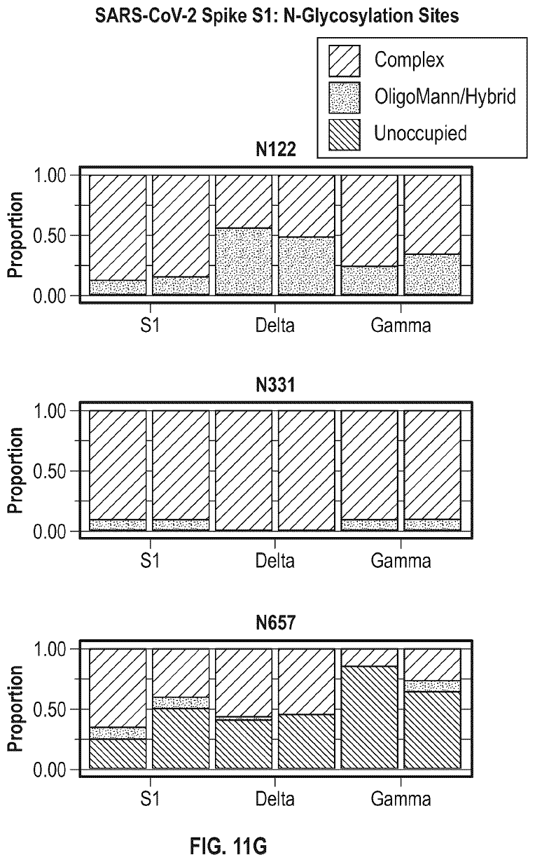

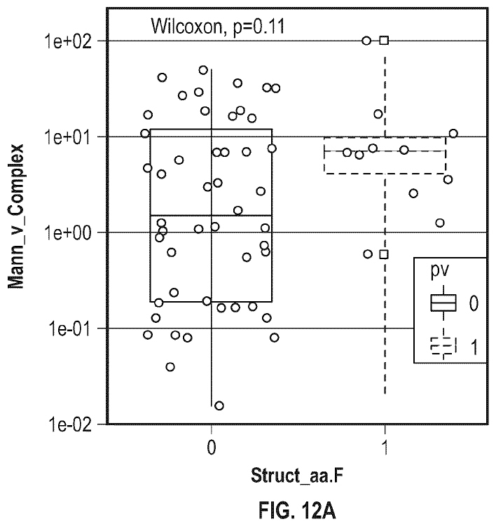

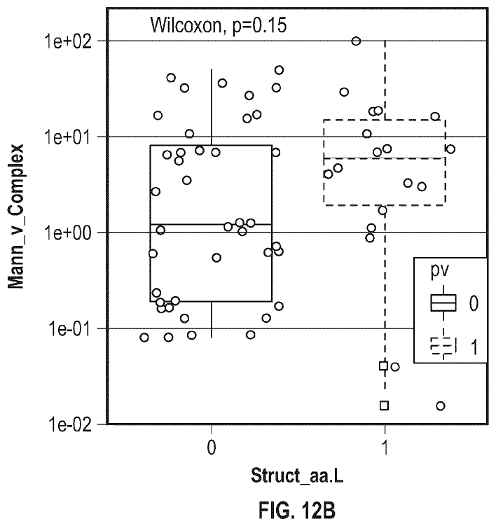

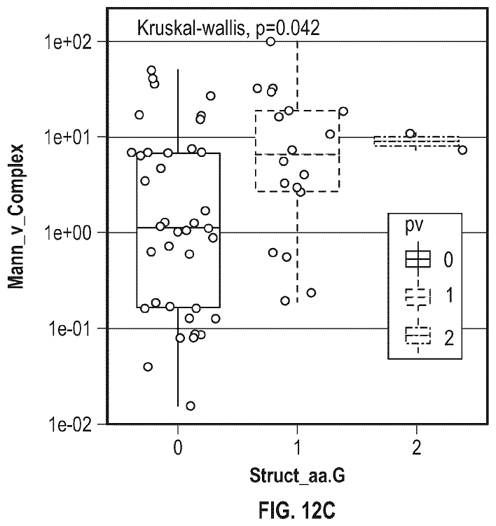

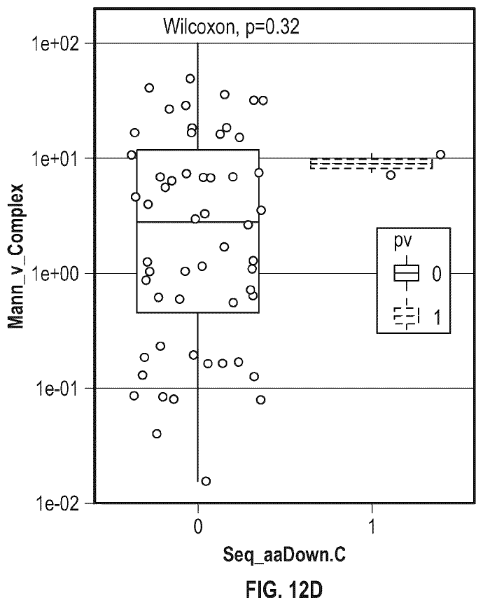

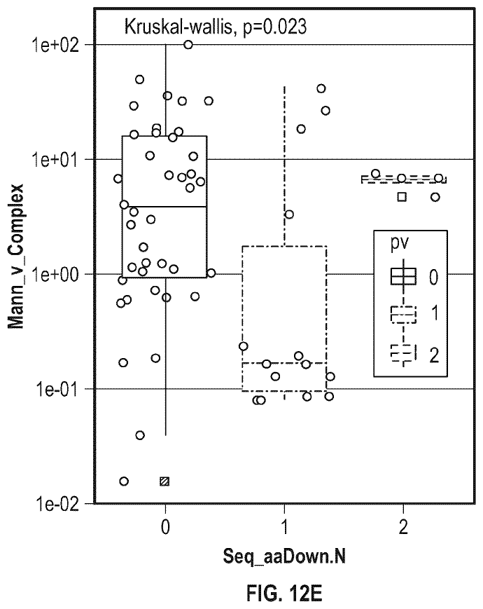

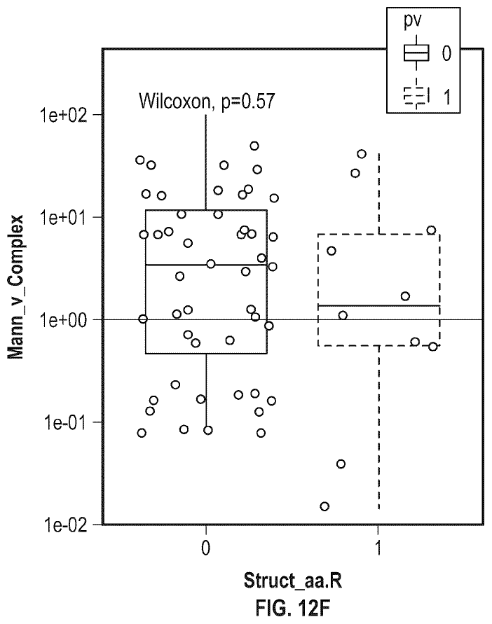

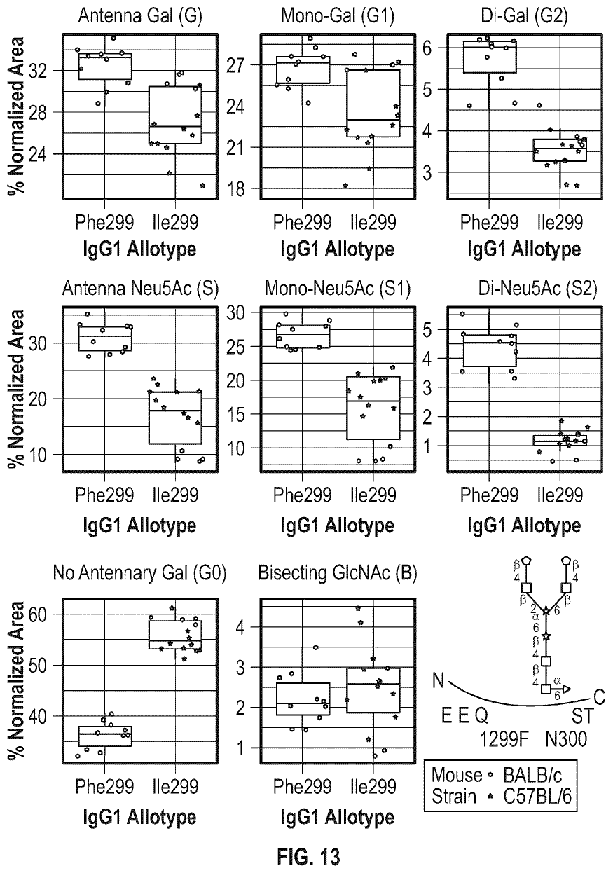

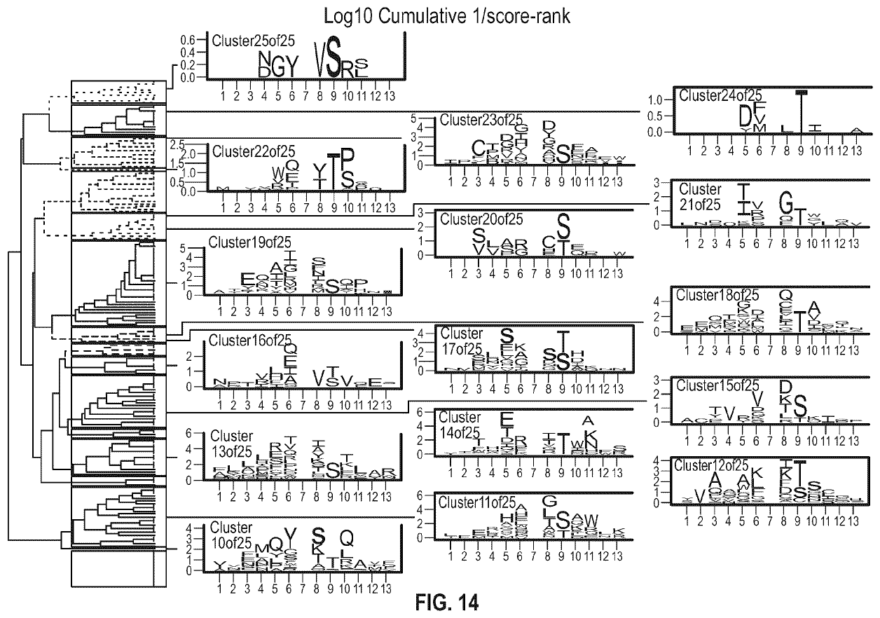

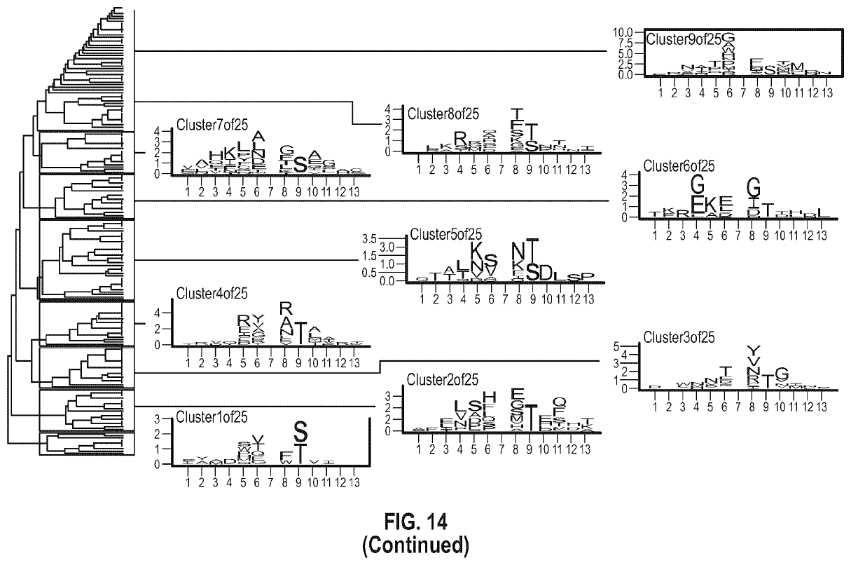

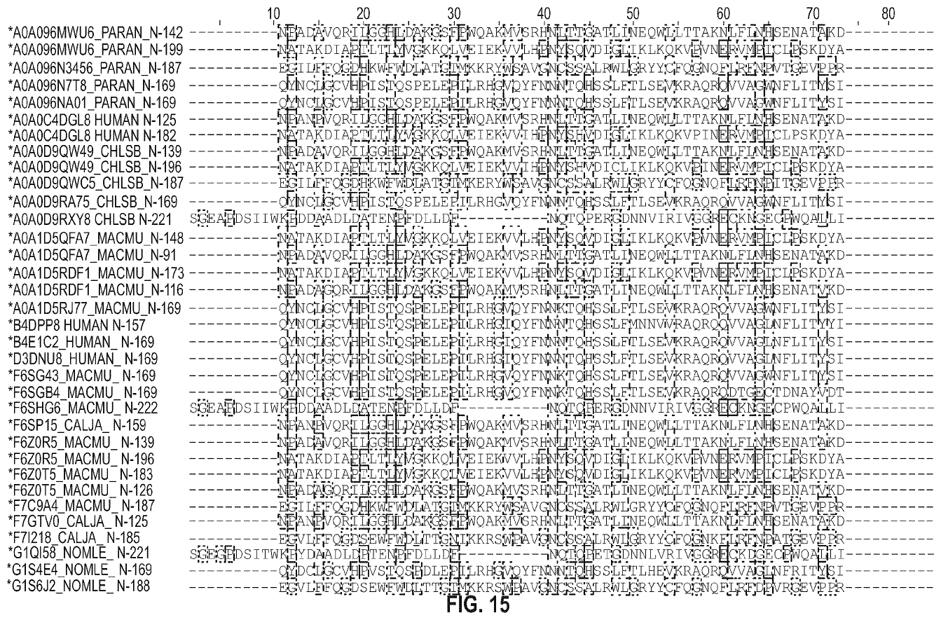

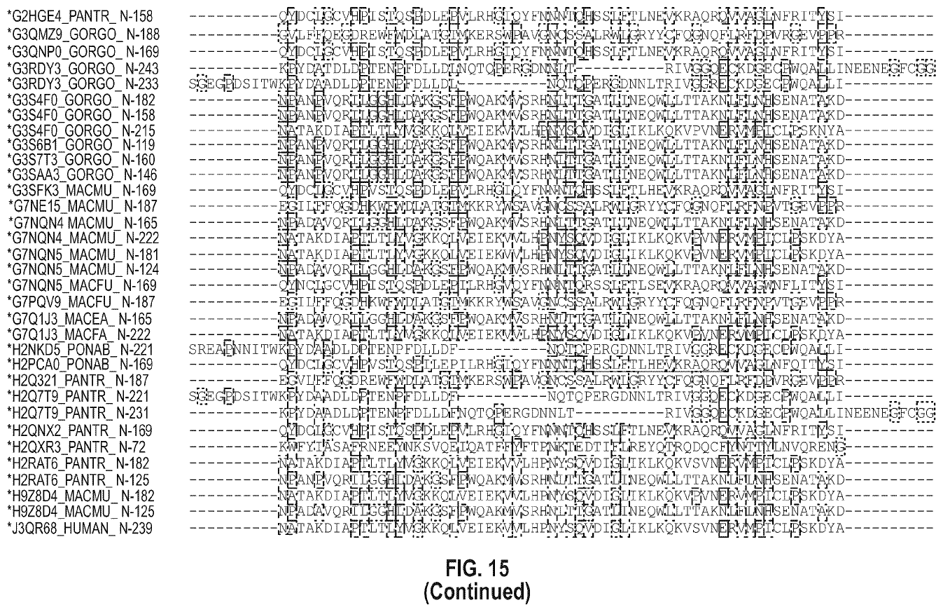

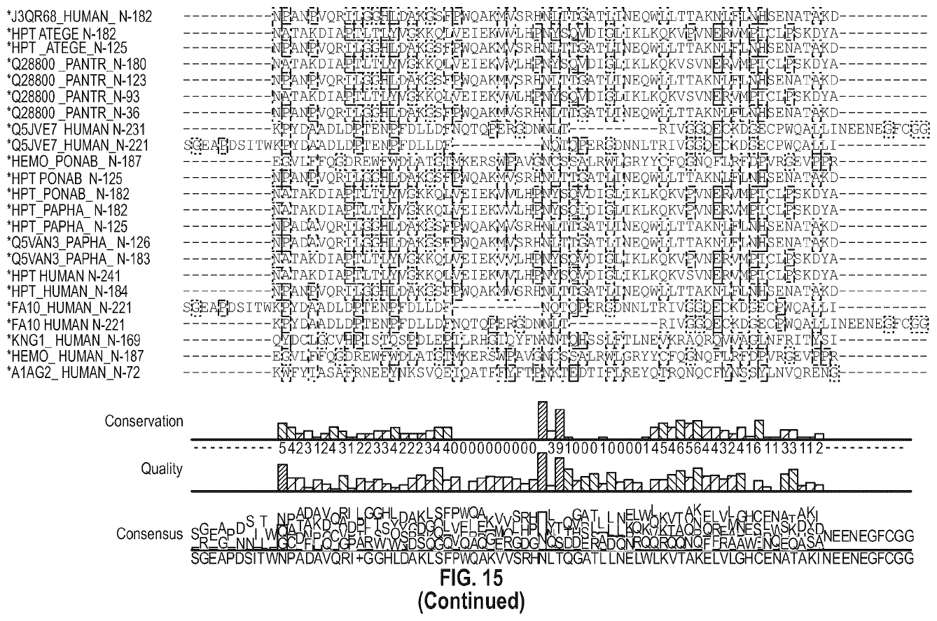

The HIV envelope proteins present several distinct N-glycans. The consistency between previously measured IMRs and HIV glycosylation was examined. PGES-DB-measured IMRs suggest that downstream glutamine was most significantly and substantially (FDR<1e−8; OR<0.5) predictive of complexity while structure-proximal Pro and Lys were weak but significant distinguishers (FDR<1e−3, FDR<0.1 respectively, FIG. 11A). These predictions' site-specific glycan complexity measurements in HIV ENV gp160 were compared (FIG. 11B). As predicted, proline-proximal (within 6 Å) gp160 glycosites presented more oligomannose (Two-sided Wilcoxon p=0.0033), whereas C-terminus-proximal glutamine were higher complexity (Two-sided Wilcoxon p=1e−4, FIG. 11C). Structure-proximal lysine, a less significant and lower magnitude prediction (FIG. 11C), revealed a nonlinear impact on glycan complexity in HIV gp160; first increasing with one proximal lysine then decreasing with two. Both of the most significant IMRs predicted from PGES-DB were consistent with the site-specific glycosylation observed in HIV gp160.

Further, differential glycosylation across Ighg1 missense mutation (Phe299Ile) in the IgG1 heavy chain was predicted. C57BL/6 and CD1 mouse strains expressing the IgG1:Phe299Ile substitution have significantly lower IgG1 sialylation and di-galactosylation than strains (e.g., BALB/c and C3H) expressing wt IgG1. Interestingly, within several BALB/c animals, heterozygous for ighg1 alleles, the Fc-linked N-glycoprofiles of IgG1:Phe299Ile were more similar to those of IgG1:Phe299Ile expressed in C57BL/6 mice, as compared to IgG1:Phe299 expressed in the same BALB/c animals (FIG. 11E-11F). The Fc-linked N-glycans of IgG1:Phe299Ile in both BALB/c and C57BL/6 animals presented increased agalactosylation (Mann-Whitney p=1.02e−6) and lower levels of di-galctosylation, mono-, di- and total sialylation (Mann-Whitney p<0.0073), as compared to IgG1:Phe299 expressed in the same animals (FIG. 11E, Table 5). The increase in galactosylation in IgG1:Phe299 is consistent with PGES-DB predicted IMRs for upstream (N-terminal) phenylalanine (FIG. 11D). Upstream phenylalanine is associated with increased di-galactosylated biantennary structures (OR>2), while upstream isoleucine is associated with tetra-antennary galactosylation. Since only bi-antennary structures are generally permitted on IgG, the Gal promotion function of upstream Ile may be unrealized in IgG. The increased sialylation in IgG1:Phe299 is also consistent with PGES-DB IMRs which show an association between structurally proximal phenylalanine and di-sialylated structures (OR>10). These results suggest that glycoimpact can accurately predict the degree of glycan complexity.

TABLE 5
P-values and FDR correction for two-sample Mann-
Whitney tests of glycan abundance distributions

| glycan | p. value | FDR |
|---|---|---|
| G | 0.000274 | 0.000353 |
| G1 | 0.007251 | 0.008158 |
| G2 | 2.04E−06 | 3.06E−06 |
| S | 1.02E−06 | 1.84E−06 |
| SI | 1.02E−06 | 1.84E−06 |
| S2 | 1.02E−06 | 1.84E−06 |
| G0 | 1.02E−06 | 1.84E−06 |
| B | 0.436617 | 0.436617 |

The SARS-CoV-2 spike S1 subunit in the original 2019 strain was compared to the Gamma and Delta variants. AlphaFold2-predicted S1 subunits were compared using pyMol (v2.5) root mean square distance (RMSD). RMSD between the 2019, Gamma, Delta and the full trimer (PDB.6VXX) were marginal (RMSD(2019,Gamma)=2.098, RMSD(2019,Delta)=6.387, RMSD(2019,Trimer)=10.435). Measuring Euclidean distance in 3-dimensions, multiple glycosite-proximal mutations (within 15 Angstroms) were found. In the Gamma spike S1, three mutations (L18F, T20N, & D138Y) appeared close to N17, two mutations (P26S & R190S) close to N61, three mutations (L18F, D138Y, & R190S) close to N122, D615G was extremely close to N616, and H655Y was extremely close to N657. In the Delta spike S1, N17 and N122 had 5 and 4 proximal mutations, respectively, and N165 and N616 each had one high-proximity mutation. Of the glycosite proximal substitutions, only one substitution in each strain had high glycoimpact; L18F in Gamma and F157V in Delta. L18F appeared within 15 Angstroms of N17, N74, N122 in Gamma. Similarly, F157V appeared within 15 Angstroms N17, N122, and N165 in Delta. Both high-impact substitutions appeared close to N17 and N122.

SARS-CoV-2 spike S1 proteins were expressed in HEK293 then glycoprofiled using the proteomics-digestion method DeGlyPHER to determine glycan occupancy (unoccupied, complex, and oligomannose/hybrid) at each glycosylation site. Two independent replicate analyses of the original 2019 strain compared to the Gamma and Delta S1 variants were performed. The proportions of unoccupied, complex, and oligomannose/hybrid observations were compared using a Mann-Whitney test, and p-values were pooled across the two independent replicate analyses using the Fisher method. Three three significant differential glycosylation events were observed (FIG. 11G). At N122, high-mannose/hybrid structures replaced complex glycans at N122 in both Delta (oligomannose/hybrid observations increased nearly 4-fold from 13.9% in S1 to 52.5%; FDR=3.3e−9) and Gamma (oligomannose/hybrid observations nearly doubled to 27.6%; FDR=7.9e−4). At N331, complex structures increased marginally to replace oligomannose/hybrid glycans in Delta (complex structures increased from 93.7% to 99.7%; FDR=0.031). At N657, complex glycosites became unoccupied in Gamma S1 (complex observations decreased by over 2-fold from 53.3% to 21.1%; FDR=1.27e3). The N17 site was inconsistently cleaved with the signal peptide precluding stable measurement in these recombinant products. The Gamma S1 monomer was consistently expressed with two novel complex glycosylation sites at N20 and N188.

Eleven of the twelve canonical S1 glycosites (excluding N17) in the original 2019 strain and the Gamma and Delta variants were examined. Significant differential glycosylation was found at N122 in both strains, N657 in Gamma, and N331 in Delta. Based on proximal high-glycoimpact substitutions, predicted change N17, N74 (Gamma only), N122, and N165 (Delta only) were predicted. Two of four (N122 in Gamma and Delta) predicted differential glycosylation events were consistent with the four observed changes (Sensitivity=0.5), while 15 sites where no change was predicted were consistent with the 17 sites where no change was observed (Specificity=0.88). The significant and substantial differential glycosylation event at N122 was correctly predicted.

### Discussion

Developing the Protein-Glycan Enriched Structure Database (PGES-DB), the correlation between protein and glycan structure were quantified, described herein as “glycoimpact.” Glycoimpact signatures were validated by comparison to substitution matrices, evolutionary couplings, and pathogenicity scores. Further validation of the glycoimpact predictions was done through comparison to glycosylation on PrP, HIV gp160, and IgG glycoproteins.

In PGES-DB, an enrichment in protein-glycan associations was inconsistent with independence. The median information gain (KLd) was substantially larger when a protein or glycan structure was present. Consistent with established glycan influence on protein folding, glycan structure may provide information-gain regarding protein structure. Yet, on average, glycans may be less determined by protein structure. Glycan size as a proxy for the influence of metabolic demand on glycan biosynthesis and steric hindrance on protein folding was examined; larger glycans (more monosaccharides) contain more opportunities for precursor-limitation. An increased conditional-marginal divergence (CMd) with glycan size was found in glycan-conditioned protein structure. The increased divergence is consistent with previous findings that glycan sterics impact protein folding. Conversely, the protein-conditioned glycan CMd decreased with glycan size, suggesting that the metabolic and processive dependencies may have an inverse impact on the predictability of glycan structure from protein structure.

Many sequence-proximal amino acid IMRs were found upstream of the glycosite, a region not previously interrogated. For example, the glycoimpact for upstream phenylalanine predicts an increase in structures containing Man7 (seven-mannose low-complexity N-glycan) and a decrease in structures containing Man6, suggesting an increase in larger high-mannose structures.

### Methods

Enrichment of glycan-protein site-matched data to generate the Protein-Glycan Enriched Structure Database (PGES-DB)

Starting from site-specific glycosylation events, the annotation of each glycosylation site and glycan was included to include detailed site-specific protein structural annotation and recorded the number of times each substructure appeared in each glycan. Only human glycoproteins were analyzed. The final database includes 111 proteins, 306 glycosylation sites and 4263 gly cans. Initially, site-specific glycosylation events documented in UnicarbKB was used. Later and current work was informed by glycosylation events documented in Glyconnect with supplemental information from GlyGen. Empirical site-specific glycosylation events from the UnicarbKB and Glyconnect were used to inform much of the core analysis.

The protein structure annotation was done using the Structural Systems Biology (SSBio) package in python. The package uses several tools to perform a variety of annotations. For each human protein, empirical and homology modeled structures were collected from the Protein Data Bank (PDB) and SWISMOD, respectively. Proteins without existing models were modelled using I-TASSER. Protein structures and chemistry close to the glycosylation sites were annotated multiple software packages through SSbio: sequence properties (EMBOS:pepstats), sequence alignment (EMBOS:needle), secondary structure (DSSP (, SCRATCH::SSpro, and SCRATCH::SSpro8), solvent accessibility (DSSP and FreeSASA), and residue depth (MSMS). Additional amino acid aggregate features were calculated using R::seqinr. Spatial proximity was defined using “min-distance” between two amino acids; the minimum distance between any pair of atoms spanning the amino acids.

Glycan structures were annotated using a combination of glypy (Klein and Zaia 2019) and GlyCompare, for structure parsing and comparison respectively. All glycan substructures, a connected subset of monosaccharides with and without linkage information, were extracted from each glycan, merged to make a superset of substructures, then mapped to each glycan, resulting in a mapping from every glycan in the input database to shared substructures.

### Software and Packages

Protein structure analysis was performed in Python v2.7.15 using SSBIO v0.9.9.8 to retrieve and calculate: existing empirical and homology models from PDB and SWISSMOD (PDBe SIFTS), de novo homology models (I-TASSER v5.1), sequence properties (EMBOS v6.6.0.0 pepstats), sequence alignment(EMBOS v6.6.0.0 needle), secondary structure (DSSP v3.0.0, SCRATCHv1.1::SSpro and SCRATCHv1.1::SSpro8), solvent accessibility (DSSPv3.0.0 and FreeSASAv2.0.2), and residue depth (MSMSv2.2.6.1). Additional amino acid aggregate features were calculated using R::seqinr.

Statistical analysis was performed in R v3.6.1. R::entropy v1.2.1 was used for entropy, Kullback-Leibler divergence and other information theoretic calculations. Generalized Estimating Equations (GEE) were fit using R::geepack v1.3.1. Gaussian Mixture Models were used to z-score normalize the glycoimpact using R::mixtools v1.1.0. BLOSUM and PAM substitution matrixes were accessed from R::Biostrings v2.52.

### Probability Event Space, Information Gain and Conditional Probability

An event (a row in the enriched glycosylation-glycosite database) was defined as as “the observation of a glycan at a glycosylation site in an experiment.” If two separate experiments in the input database both observe the same glycan at the same site on the same protein, that event was considered to have occurred twice. Within each event, it was considered if the glycan structure random variable (the presence or absence of a specific glycan substructure) is present or absent in the observed glycosylation event and if the protein structure random variable (a proximal amino acid, a secondary structure or another discrete protein structure). A Fisher exact test (R::base::fisher.test) was used to estimate the odds ratio (OR) and significance (p) of each inter-molecular relation (IMR). P-values were corrected for False Discovery Rate (FDR, q) permitting 10% false discovery (q<0.1). Conditional probability was calculated by dividing joint probability by the marginal probability of protein and glycan structure presence. Kullback-Leibler divergence (KLd, R::entropy::KL.Dirichlet, pseudo count=1/6) was calculated by comparing the conditional probability distribution to the marginal probability distributions.

### Quantitative Characterization of Inter-Molecular Relations (IMR) Using Generalized Estimation Equations (GEE)

To characterize the IMRs in the PGES-DB while controlling for protein-specific confounding effects and handle nonlinear relations, a population-averaging approach was used: logistic Generalized Estimating Equations (GEE) with glycoprotein identity as the cluster identifier. An exchangeable correlation structure was used to describe and balance the in-protein similarity. Models were fit to predict glycan substructure binary (presence or absence) from either z-score normalized continuous or binary (presence or absence) protein structures. For each model, the data from PGES-DB was isolated for one glycan-type (N-glycan or O-glycan), one glycan substructure and one protein structure. Incomplete observations (events/rows) were removed and then several checks on each data-slice were run to minimize overfitting. Glycan substructures were excluded from modelling if standard deviation was less than 1e−6 or if there were fewer than 5 observations of the structure within the pertinent data-slice. Discrete protein structure features were excluded if there were fewer than 4 observations within the data-slice. Models were excluded if there were fewer than 4 instances in any cell (of the 2×2 absence/occurrence matrix) or if the chi-squared expected value of any cell was less than or equal to 5. Observations were weighted by the reciprocal-count of the corresponding label type to balance label contributions to the model and scaled by exponentiated cscore to maximize the contribution of high-quality protein structure models ( ); c is the c-score given by I-TASSER and n is the number of times a structure is present (1) or absent (0). Models with |log(OR)|>50 were excluded as likely overfit. Quasi-likelihood under independent model criterion (QIC) and the Wald tests were used to evaluate the significance and magnitude of the estimated IMRs. This analysis was run using publication identifiers as a control variable to account for researcher and group biases; this produced similar results likely because protein identity is strongly correlated with the publications in which they appear.

Calculating Glycoimpact from IMRs and Populating a BLAMO Matrix

Glycoimpact is calculated for every pair of AAs as the Euclidean distance between significant and substantial log ORs for each AA; the Euclidean distance between expected glycoprofiles for each AA. The substantial (log(OR)>X) and significant (FDR<Y) log(OR) values are retained while insignificant or unsubstantial log(OR) values are set to zero. The resulting matrix describes the expected glycoimpact due to each AA-substitution, termed the BLAMO XY matrix where X and Y denote the log(OR) and FDR thresholds respectively.

Glycoimpact values from a BLAMO XY matrix may then be z-score normalized to a Gaussian Mixture Model estimated null distribution. z=2.5 may be used as a heuristic, but stringent, cutoff between “impactful” (z>2.5) and “null” (z<2.5) substitutions.

Comparison of SNP Pathogenicity Scores with Glycoimpact

Functional prediction rank normalized scores were obtained from dbNSFP (v3.2) for the following 27 tools: SIFT, PolyPhen-2 HDIV, PolyPhen-2 HVAR, GERP++, MutationTaster, Mutation Assessor, FATHMM, LRT, SiPhy, 2×PhyloP, MetaSVM, MetaLR, CADD, VEST3, PROVEAN, 4× fitCons scores, fathmm-MKL, DANN, 2× phastCons, GenoCanyon, Eigen and Eigen-PC. Variants were excluded from the analysis if they had more than 3 missing functional score predictions, did not result in an amino acid change, or not on proteins that had known glycosylation sites.

Assignments of “prediction-type” and “structure-usage” were adapted from classifications provided by dbNSFP.

### Estimation and Analysis of Evolutionary Coupling (EC)

For EVCouplings calculation, hits of more than 50% gaps were filtered from the alignment, and sequences with homologs more than 80% identical were downweighted to compute Neff, the effective number of sequences. ECs were calculated using pseudo-likelihood maximization, as implemented previously. The λ_{J} term was scaled by the number of amino acids minus one times the number of sites in the model minus one. Pre- and post-processing was performed using the EVCouplings Python package.

High-ranking EC events are generally considered those ranking less than L, the alignment length within the corresponding protein. Multiple high-rank thresholds between L 5 and 3L were explored. To explore the increased coupling with glycosylation sites, couplings between each amino acid with glycosites (GN), asparagines (N) and any amino acid (AA) were examined. The number of high-ranking coupling events, the distributions of EC probabilities and the relative numbers of high and low-ranking ECs for each group were compared with various amino acids at relative positions N+/−6. Distributions were compared with a one-sided Wilcoxon test, and high/low-ranking counts were compared with hypergeometric enrichment. The hypergeometric enrichment of glycosite-coupling was performed at multiple high-rank thresholds (L 3, L 2, L, 2L, 3L), and p-values were pooled for each amino acid at each relative position across ranks using Fisher's method. Finally, the pooled p-values were corrected for multiple tests using the Benjamini-Hotchberg method.

To examine larger structures in ECs, EC rank was used to mask extended sequons (N+/−6). The sequons were then clustered and motifs extracted. For each sequon, the residues were retained if the residue-glycosite coupling rank was less than L4. The extended and masked sequons were distinguished using a hamming distance (DECIPHERv2.18.1) then clustered using agglomerative hierarchical clustering (factoextra::hcut v1.0.7). Motif logos were generated using custom-scaled position-specific scoring matrices (Wagih 2017) reflecting the cumulative rank of amino acids at each glyco-site relative position. Specifically, the aggregate score, S, for each amino acid, a, at each position, p, was aggregated over EC score ranks, r, within each extended-masked sequons, s, in a cluster, c, such that

### Mouse Breeding and Samples

The Collaborative Cross (CC) recombinant inbred mouse strains (N=333, 95 strains, age 20-117 weeks) were produced by Geniad Pty Ltd and housed at Animal Resources Centre (Murdoch, WA, Australia). The CC strains were genotyped using the MegaMUGA platform (GeneSeek; Lincoln, NE). C57BL/6 (N=10) and BALB/c mice (N=10), sex- and age-matched (10 weeks old, 1:1 male:female) were obtained from Elevage Janvier (Le Genest-Saint-Isle, France). The studies received appropriate ethics approvals from the Animal Ethics Committee of the Animal Resources Centre and the Ethical Committee of the District Government of Lower Franconia.

### Liquid Chromatography-Mass Spectrometry (LC-MS), Normalization and Statistical Analysis of Mouse Fc-Linked IgG N-Glycopeptides

Immunoglobulin G was isolated from 100-500 μl of mouse serum on 96-well Protein G monolithic plates (BIA Separations) as described previously. LC-MS analysis of tryptic Fc-glycopeptides was performed as described in. In brief, approximately 10-20 g of isolated IgG was digested with 200 ng trypsin (Worthington, USA). The resulting glycopeptides were purified by reverse-phase solid phase extraction using Chromabond C18ec beads (Marcherey-Nagel, Germany). Tryptic digests were analyzed on a nanoACQUITY UPLC system (Waters, USA) coupled to a Compact mass spectrometer (Bruker Daltonics, Germany). Peak areas were calculated by summing areas for doubly and triply charged ions determined with LaCyTools v 1.0.1 b.7 software and normalized to the total integrated area per IgG subclass.

Batch correction was performed on the log-transformed values using the ComBat method (R package “sva”) to remove possible experimental variations due to LC-MS analysis having been performed on several 96-well plates within each cohort. Derived glycosylation traits describing relative abundance of N-glycans sharing specific structural features (agalactosylated, galactosylated, sialylated, monogalactosylated, digalactosylated, monosialylated, disialylated structures, structures with bisecting GlcNAc) were calculated in a subclass-specific manner. Statistical analysis and data visualization were performed using R programming language v 4.0.3.

### Example 2. Prediction of Changes in Glycosylation of Anti-Ebola Fc Antibody

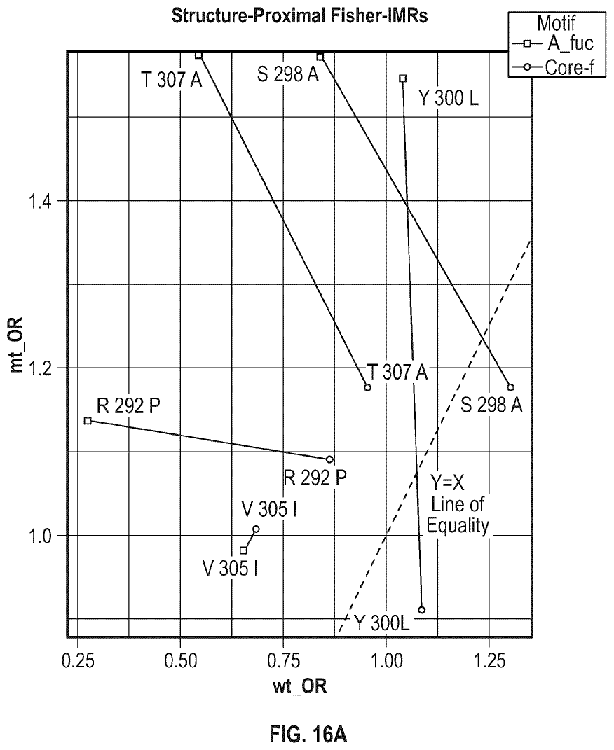

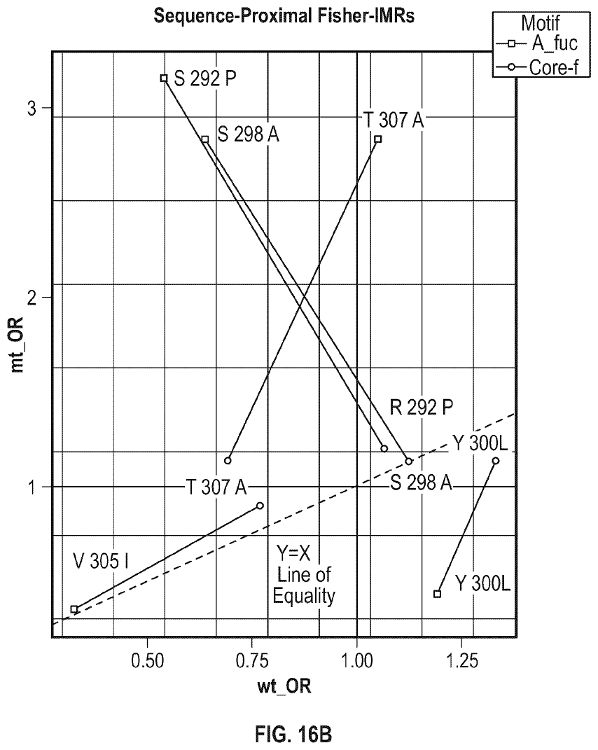

To determine if predicted changes in glycosylation can also predict glycan-modulated behaviors, the increases in antibody-dependent cellular cytotoxicity (ADCC) were predicted from predicted decreases in core-fucosylation. Anti-ebola virus antibodies from convalescent plasma were characterized for differential immune modulation. Of these antibodies, five (R292P, S298A, Y300L, V3051, T307A) contained Fc-variation close to the N297 (PODOX5) glycosylation site and showed increases in ADCC or FcRn binding. For each allotype, the relative glycoimpact (Fisher-OR estimated structure and sequence-proximal IMRs) was queried for each wt and mutant amino acid (e.g. R and P respectively upstream of a glycosite) on the N-glycan core motif with (Man(b1-4)GlcNAc(b1-4)[Fuc(a1-6)]GlcNAc(b1-4)-Asn; X183) or without (Man(b1-4)GlcNAc(b1-4)GlcNAc(b1-4)-Asn; X69) a core fucose. Wildtype and mutant glycoimpacts were plotted against each other for structure-proximal FIG. 16A) and sequence-proximal (FIG. 16B) effects. In 4 of 5 allotypes structure-proximal IMRs indicate an increase in afucosylated structures (above y=x, equal likelihood in wt and mutant) while core-fucosylation likelihood remains close to equal (FIG. 16A); 3 of 5 allotypes show the same behavior in sequence-proximal IMRs (FIG. 16B). The relative increase in predicted afucosylation for each ADCC-increasing allotype was consistent with the increase in ADCC observed.

### Example 3. A Trained Algorithm for Predicting Glycans

Described herein are trained algorithms (e.g., machine learning algorithms) such as the Interloping Saccharide Neural Network Extrapolation (InSaNNE), a machine learning model predicting glycosylation given glycosite-proximal protein structure. Using long short-term memory (LSTM) units, within a recurrent neural network, the functional and biosynthetic glycan encodings of SweetTalk, SweetNet and GlyCompare were leveraged to generate an accurate mapping to protein sequence and structure. Several protein structure models and resolutions were explored for performance including empirical, curated and ab initio homology models. The glycosite-glycan pairing model was trained and validated on empirically observed site-specific glycosylation events from UnicarbKB and GlyConnect respectively. Predictions on a small number of important glycosylation events on the coronavirus spike protein, immunoglobulin, and the enhanced aromatic sequon validate the trained model.

### Results

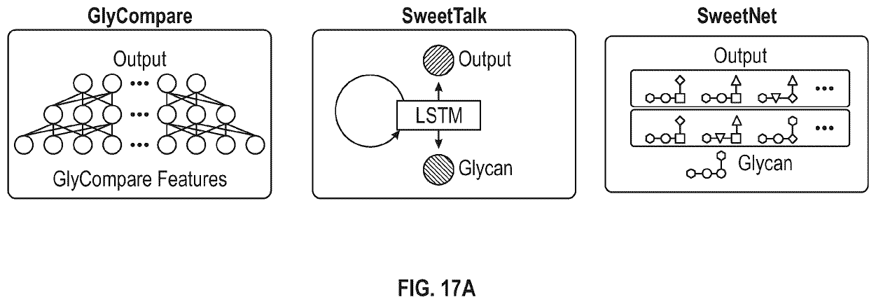

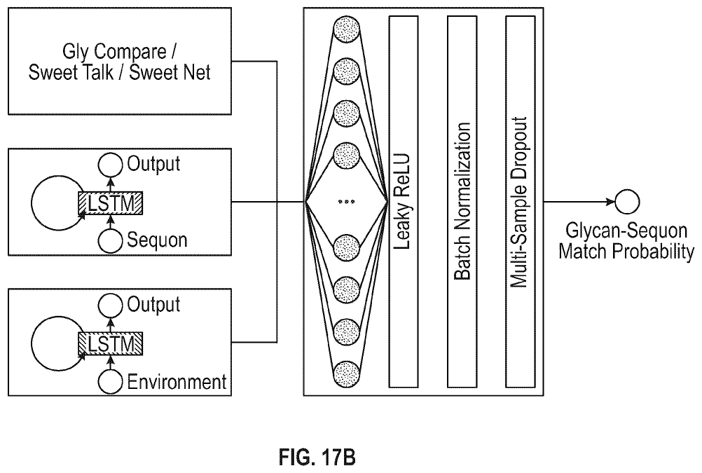

To predict which glycans will or could be present on a protein, a model was developed to predict whether a given sequon will be glycosylated with a given glycan structure. Provided with such a trained matching model, a list of available glycans (for instance tailored to the species of interest) could then be used as input to determine which glycans are predicted to be permissible with a sequon of interest. Representative models investigated are shown in FIG. 17A.

To analyze the protein sequences, long short-term memory (LSTM) units, a module used in recurrent neural networks that treated protein sequences as a biological language was used (FIG. 17B). Two separate LSTM-based modules were integrated into the model for analyzing the sequon and spatially proximal amino acids, respectively. For the analysis of the glycan component, several different modules were tested: (1) a fully connected neural network that used the GlyCompare features of a glycan as input, (2) a glycan-based language model in the style of SweetTalk, and (3) a graph convolutional neural network based on SweetNet.

On average, the model based on GlyCompare features achieved an accuracy of ˜79.7% in predicting whether a given glycan could be found on a specific glycosylation site (Table 6). Models based on recurrent neural networks (˜84.5%) or graph convolutional neural networks (˜87.1%) further improved on this competitive performance, demonstrating that optimizing the module analyzing glycan sequences yields a substantial increase in prediction performance. This result points to the information-richness of glycans that can be leveraged with state-of-the-art machine learning algorithms. Choosing the SweetNet-based model for further refinement, stochastic weight averaging was used to further optimize performance. In this technique, models at multiple training time points were averaged to yield more robust and generalizable models at the end of the training process. Indeed, SweetNet-based models enhanced with SWA achieved an average prediction accuracy of ˜90.6% and represented the final model, InSaNNe, which was used for downstream analyses.

After optimizing the analysis of glycan sequences for the model, the role of protein sequences on prediction performance was analyzed. For this, a model was trained that only had access to the glycan sequences and sequons, without additional spatially proximal amino acids. Compared to the full InSaNNe model (˜90.6%), this ablated model achieved a slightly worse performance (˜89.1%, Table 6, “-Environ”), suggesting there may be relevant information in the three-dimensional context of glycosylation. However, even without access to the spatially proximal amino acids, InSaNNe still retained most of its predictive performance. Such models may be useful in cases where no structural information is available.

A model was further trained that, in addition to sequon, proximal amino acids, and glycan sequences, also had access to the full protein sequences. The effect of this additional information on performance is shown in Table 6 below (˜90.8% accuracy, Table 6, “+Whole”).

TABLE 6
Developing a model for glycan-sequon matching to predict
permissible glycans on a glycosylation site.

| | | | | | SweetNet | SweetNet |
|---|---|---|---|---|---|---|
| | | | | SweetNet | SWA − | SWA + |
| Metric | GlyCompare | SweetTalk | SweetNet | SWA | Environ | Whole |
| Accuracy | 0.797 | 0.845 | 0.871 | 0.906 | 0.891 | 0.908 |
| ROC AUC | 0.789 | 0.850 | 0.869 | 0.900 | 0.888 | 0.901 |

Given the remarkable diversity of glycans, performance of the model on different classes of glycans was analyzed. Considering differences in sequons associated with N- and O-linked glycans, the trained model was first tested on both N- as well as O-linked glycans. InSaNNe achieved approximately equally high performance on both N- (96.2% accuracy) as well as O-linked glycans (99.8%) in the dataset, despite the consensus glycosylation sequence of O-linked glycans exhibiting far more diversity than that for N-linked glycans. The high accuracy in these cases, compared to the overall accuracy of ˜90.6% that also included predicting non-matches, suggests that InSaNNe exhibits high recall—recovering nearly all permissible glycans at a given sequon.

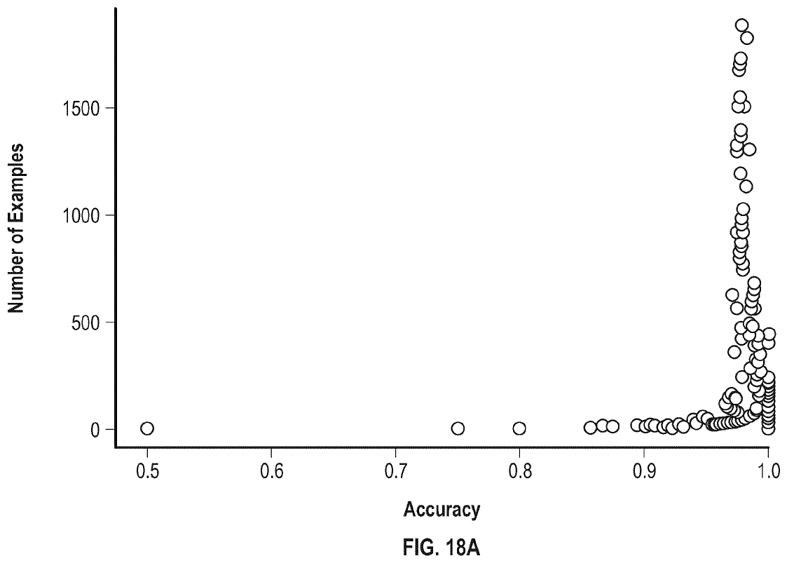

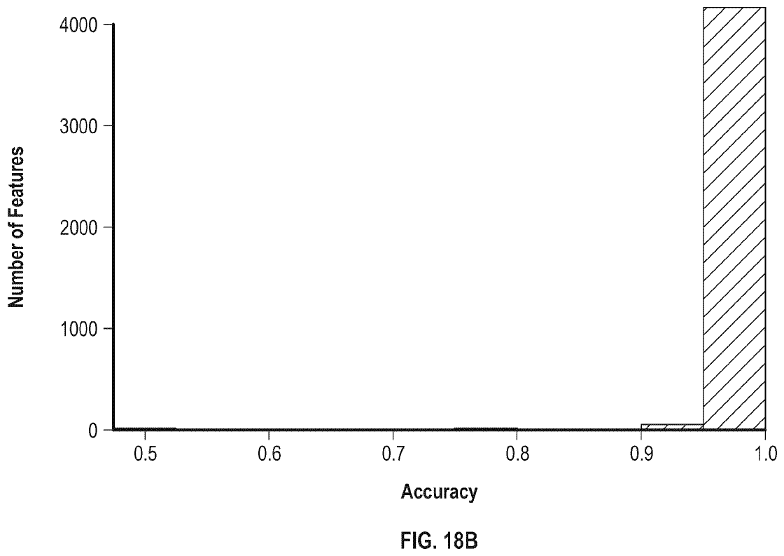

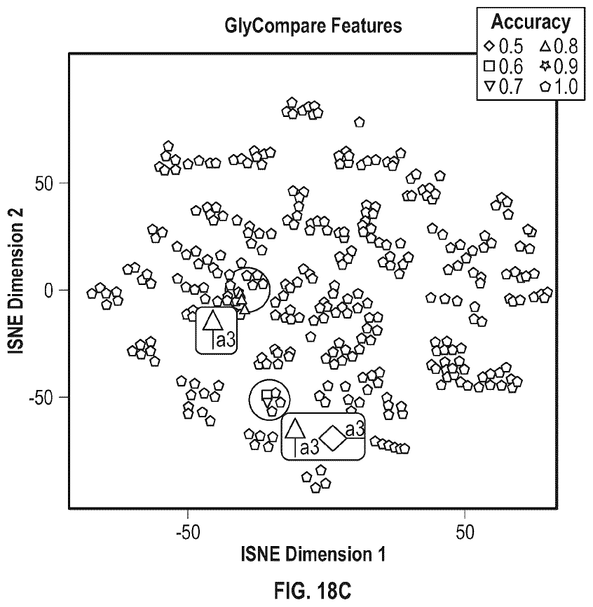

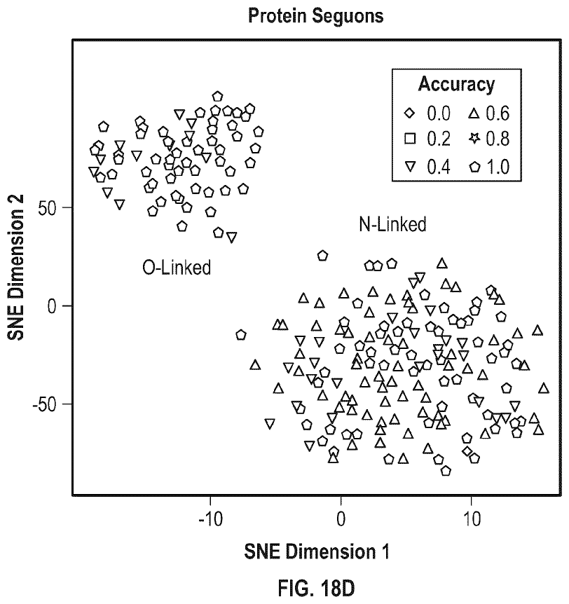

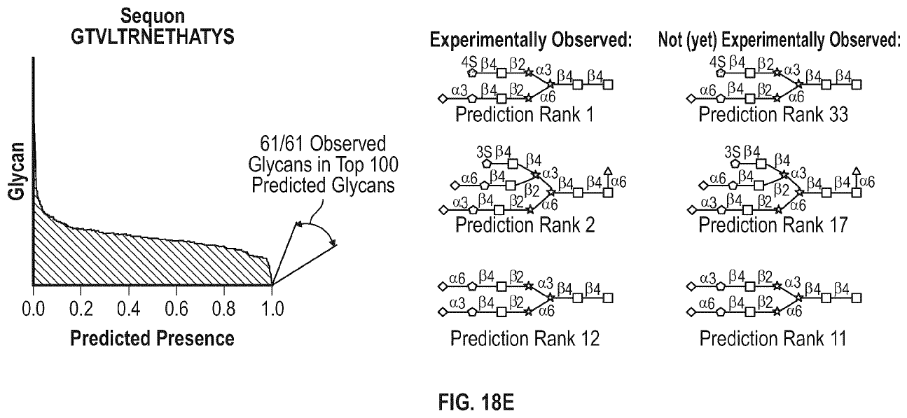

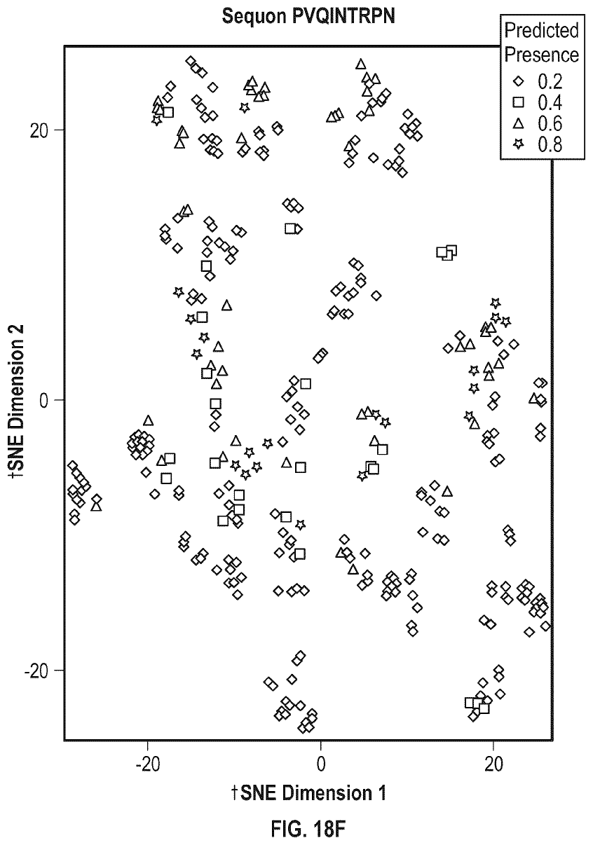

To determine if any glycan motif were especially difficult to predict, the average prediction accuracy of the trained InSaNNe model for each GlyCompare feature was calculated. Rare GlyCompare features exhibited a lower prediction accuracy than more frequent motifs, as InSaNNe had more examples and data to learn from for the latter case (FIG. 18A). However, very few examples could result in good prediction performance, and InSaNNe exhibited a predictive accuracy of more than 90% for nearly all motifs (FIG. 18B).

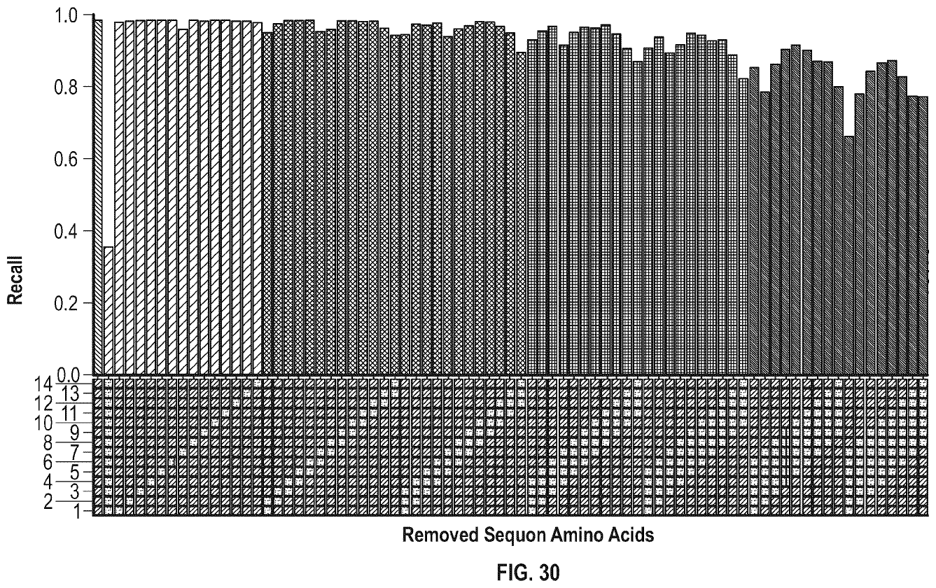

In one example, ten examples were sufficient to ensure an accuracy of at least 90% with the trained model. As GlyCompare features represent a hierarchical feature set, rare motifs with low prediction accuracy may not be independent from each other and demonstrated clusters based on their sequence similarity (FIG. 18C). GlyCompare features with lower predictive performance were enriched in α1-3 linked fucose, a glycan modification commonly found in plants and O-linked glycans. Analogous to the glycan features, most sequons exhibited an aggregate predictive accuracy beyond 90% (FIG. 18D), a correlation of prediction performance with the number of observed glycans for that sequon was again observed (FIG. 29). Widespread redundancy in the information extracted from sequon sequences was observed, as removal of single amino acids or short motifs only had a negligible impact on prediction performance (FIG. 30). The flanking residues, rather than the central region that contained the glycosylation consensus sequence, were found to inform model predictions, with a potentially stronger impact of the upstream flank (FIG. 30).

To illustrate the capabilities of InSaNNe with an example, the sequon GTVLTRNETHATYS (SEQ ID NO: 62) from human uromodulin, the most abundant protein in human urine and relevant for chronic kidney disease, was selected as an example. The trained InSaNNe model was used to predict the probability of all glycans in the dataset to decorate this sequon. All 61 experimentally observed glycans were placed in the top 100 predicted glycans (FIG. 18E). Additionally, glycans in the top 100 that were not yet experimentally reported in conjunction with this sequon shared features with the observed glycans, such as a strong negative charge via sialylation and/or sulfation. These results demonstrate that glycosylation range may be accurately predicted by models described herein.

Next to predicting specific glycans, the model was also able to predict motifs that are likely to be present in glycans at a given sequon. This feature is relevant as it may permit users to select motif-specific lectins to probe biological samples. The sequon PVQINCTRPN (SEQ ID NO: 63) from human immunodeficiency virus (HIV-1) envelope glycoprotein was used as the input for the trained InSaNNe model. As sequons from HIV-1 proteins were not part of the dataset for developing InSaNNe, this served to validate the model. This procedure resulted in structurally related clusters of predicted glycans (FIG. 18F) that can be compared between different sequons (FIG. 18G).

Additionally, directly predicting glycan motifs or features facilitates statistical enrichment analyses to determine which motifs are significantly enriched or depleted in this specific glycosylation site. The GlyCompare features of all glycans in the dataset were determined, and the InSaNNe model was trained to predict which GlyCompare features were present in a glycan associated with a sequon. Then, the enrichment of motifs was assessed by a one-sided Wilcoxon rank-sum test, a non-parametric test in which the ranks of predicted glycans were compared with that feature against the null hypothesis that they are randomly distributed across all ranks. If a GlyCompare feature obtained a p-value of smaller than 0.05 (corrected by the Benjamini-Hochberg false detection rate correction), it was deemed significantly enriched in glycans predicted to be present at that sequon.

The trained InSaNNe model, which has learned the relationship between sequons and glycan ranges, was then used to probe what effect changes to the sequon would have on predicted glycans.

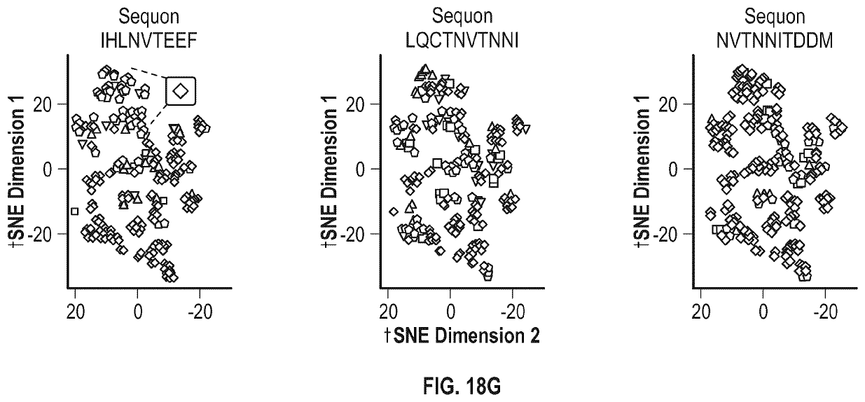

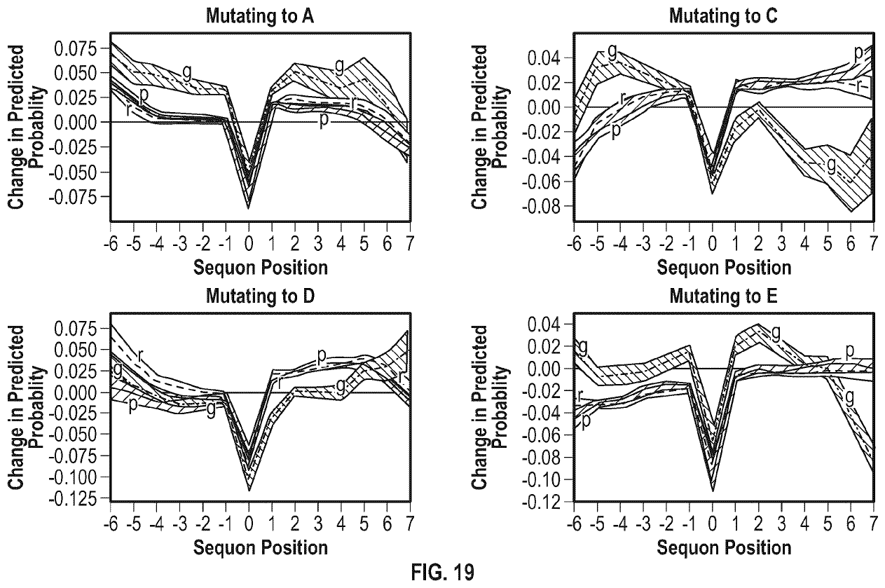

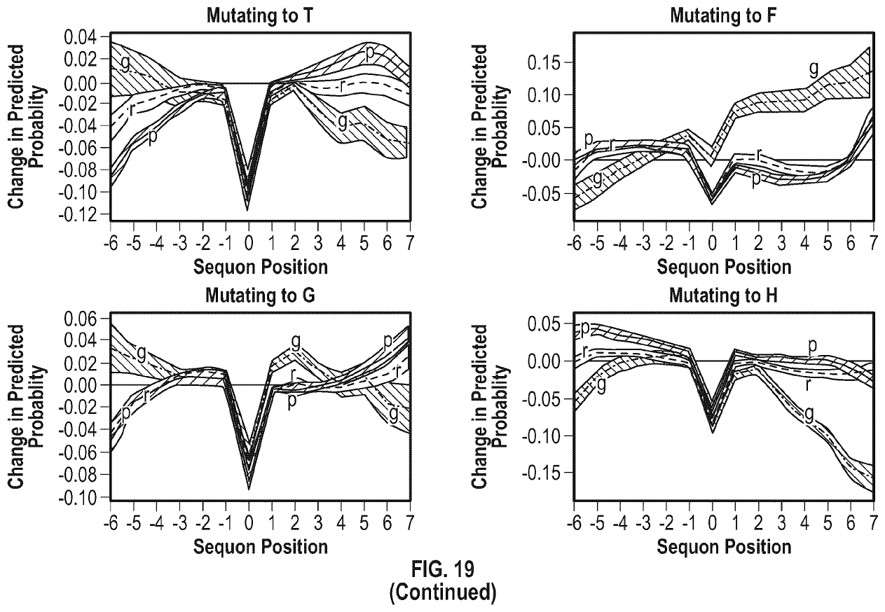

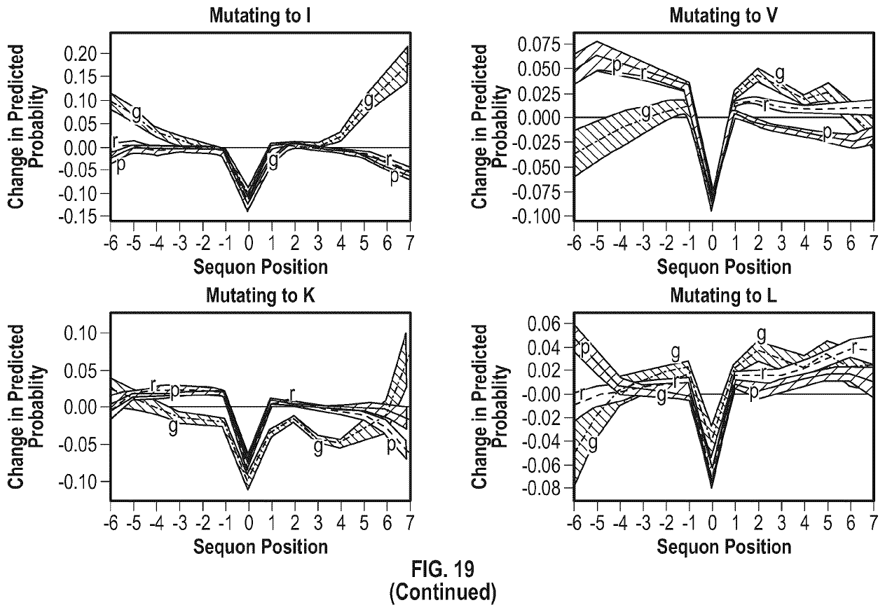

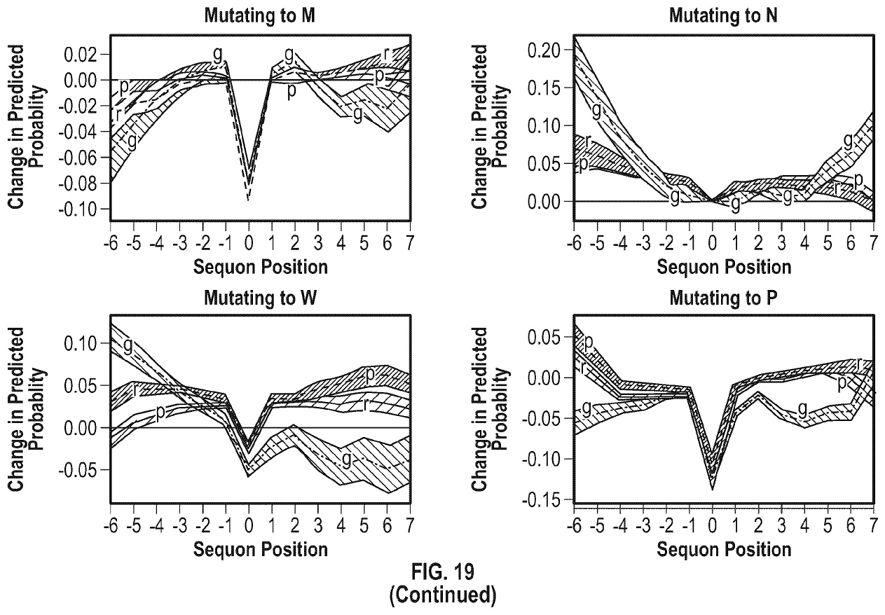

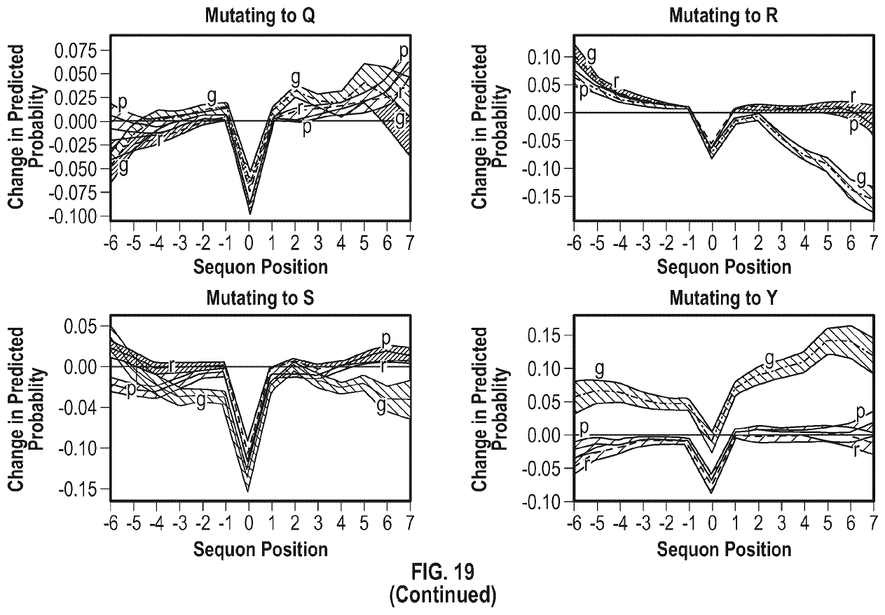

As O-linked glycans exhibit a considerably different sequon architecture, N-linked sequons were focused on for this analysis. Then, for each position in a 14-amino acid sequon, all amino acids were iteratively replaced with a given amino acid in all sequons in the dataset. These modified sequons were used as inputs for the InSaNNe model, and the changes in predicted glycans compared to the wild-type sequence were assessed. To aid interpretation, glycans were grouped into “high-mannose”, “sialylated”, and “fucosylated” to analyze common features that are relevant for human glycobiology. Changes in predicted probability for each of these features when modifying a given amino acid at different positions of the sequon were thus tracked (FIG. 19).

For multiple amino acids, distinct changes in the predicted glycosylation range of modified sequons were observed, with a clear difference between changes to upstream and downstream regions, respectively. While the introduction of some amino acids (e.g., tyrosine) had the same qualitative effect regardless of where they were introduced, other amino acids (e.g., cysteine) seemed to have diverging effects, with an increase in high-mannose glycans when introduced upstream and a decrease when introduced downstream. While there is a clear difference between upstream and downstream, within these two regions the effect seemed to be large monotonous, albeit with some changes in the direction of predicted effects (e.g., glutamate first increasing high-mannose when introduced downstream and then, further downstream, decreasing high-mannose).

One possible advantage of predicting glycosylation is a considerable increase in scale and speed for characterizing protein glycosylation, especially in the context of newly discovered proteins, for which obtaining glycan information might otherwise take several months. So, the utility of InSaNNe as an annotation tool for protein glycosylation was next demonstrated.

One possible predictions in this realm is the high-confidence prediction of sequons that will be modified by N-linked glycosylation. To leverage and extend this capability, the glycosylation ranges of 2,763 human N-linked sequons deposited in the database GlyConnect were predicted, focusing on human proteins given that the model was trained on human data.

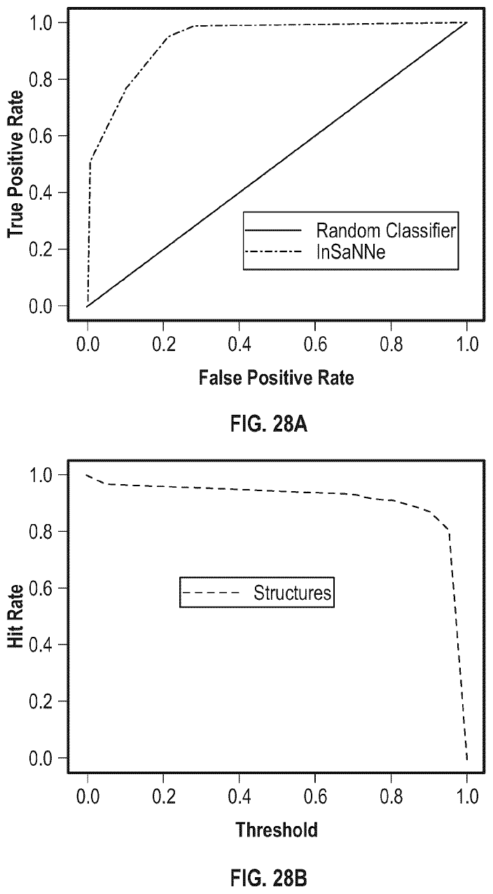

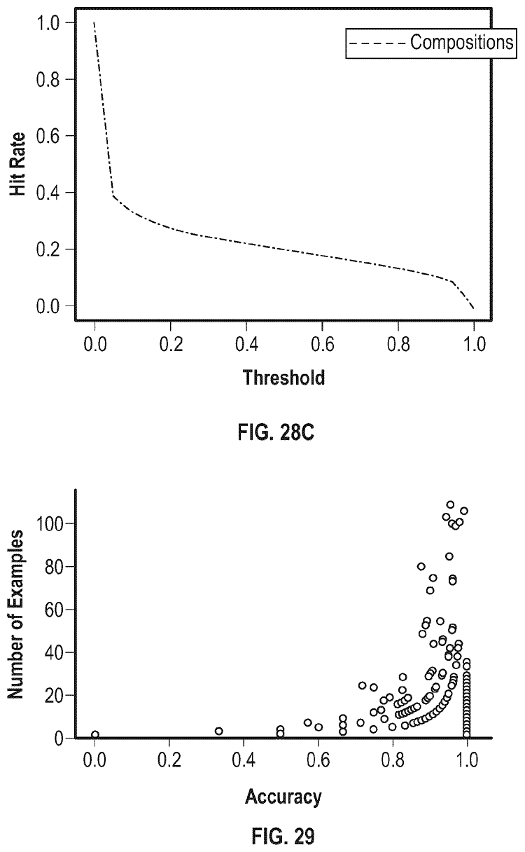

For this, annotated glycosylation sites together with the six upstream and seven downstream amino acids were extracted, resulting in sequons that were used as inputs for the trained InSaNNe model. Then, for each sequon, the likelihood of the 199 N-linked glycans in the dataset were predicted. A threshold of 0.6, corresponding to a false-positive rate of below 10% while still maintaining a true positive rate above 75%, was chose (FIG. 28A).

With that, the hit rate (also known as recall or sensitivity) of the predictions within GlyConnect was assessed by considering how many already recorded structures were matched by structures (FIG. 28B) and how many compositions could be explained and refined by InSaNNe predictions (FIG. 28C).

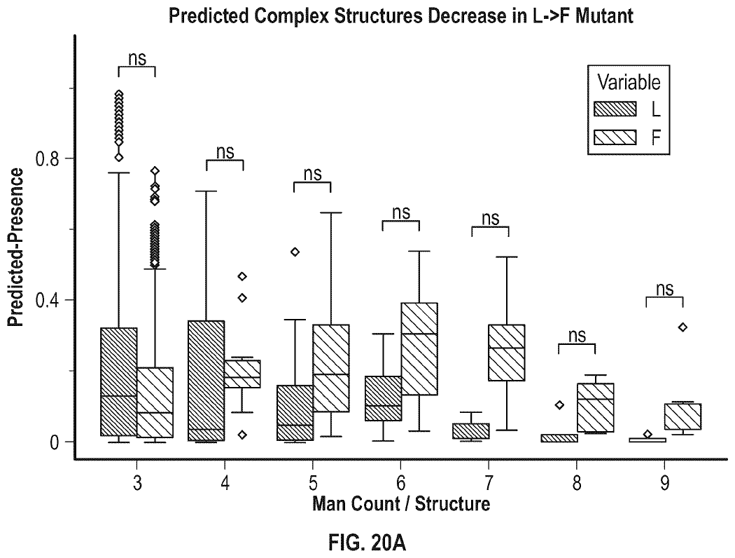

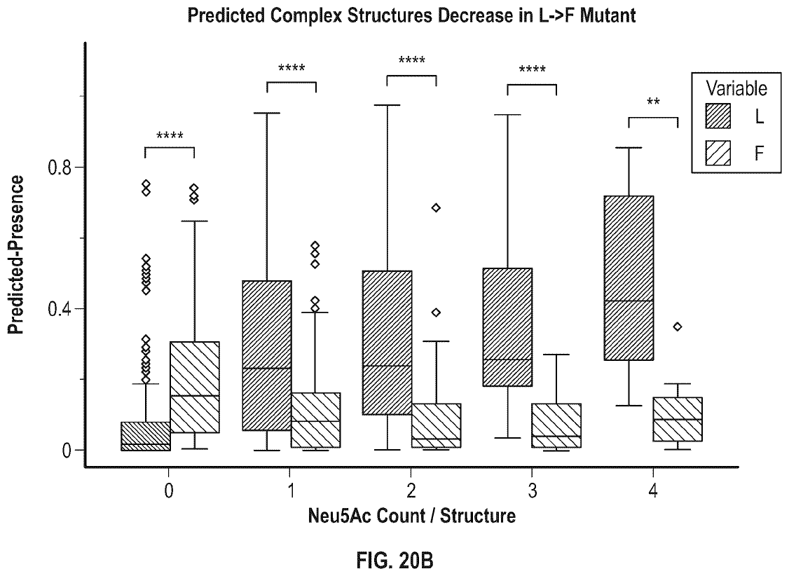

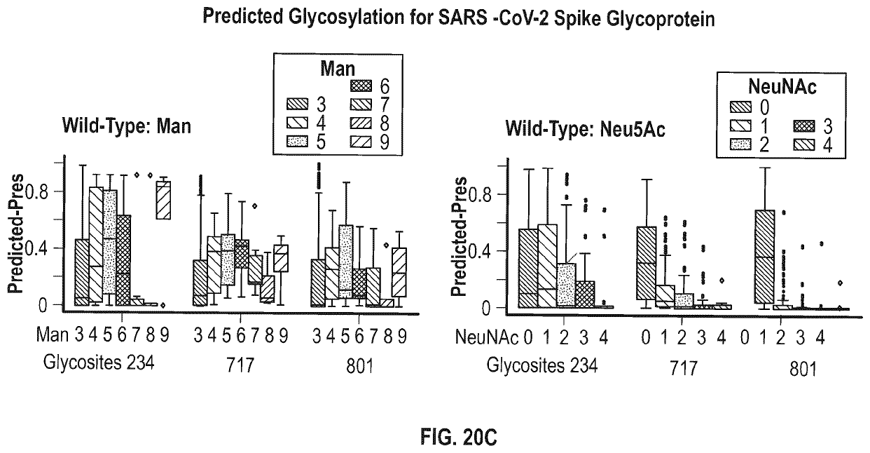

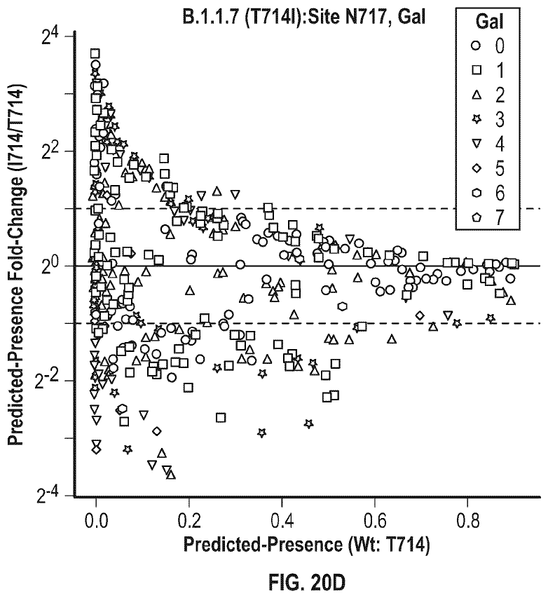

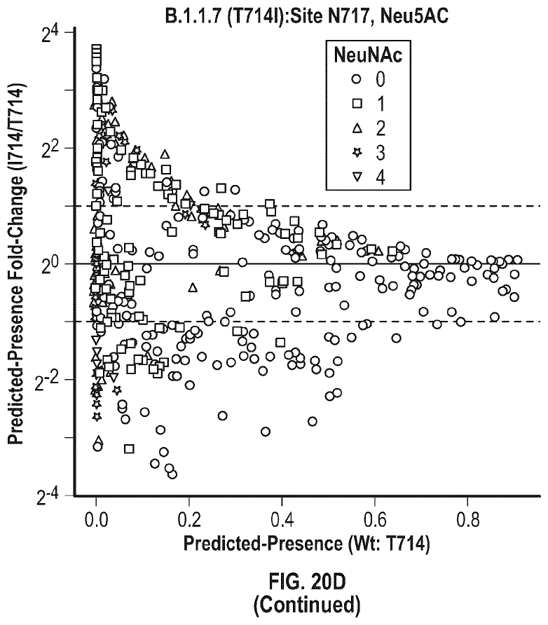

One most fundamental distinction between glycans is between highly processed, complex, and immature, oligomannose, glycans. It has been reported that an aromatic residue 2-positions N-terminal from a glycosylation site would decrease complexity at the site; this sequon has been termed the enhanced aromatic sequon. An L to F substitution two residues upstream of the CD2 glycosylation site may transform the site from predominantly complex and hybrid structures to oligomannose structures. When InSaNNe evaluates the same sequences, the F allele sequence shows significantly higher predicted presence for higher-mannose structures. An individual enrichment for 7-mannose structures (One-sided Mann-Whitney-Wilcoxon, p=0.017) and an overall increase in oligomannose structure predicted-presence for the F allele (Linear model; Wald. p<0.001; F-statistic, p=7.44×10⁵; FIF. 20A) were observed. A corresponding decrease in predicted-presence for sialylated structures in the F allele (One-sided Mann-Whitney-Wilcoxon, p<1e−4; FIG. 20B) was also observed.

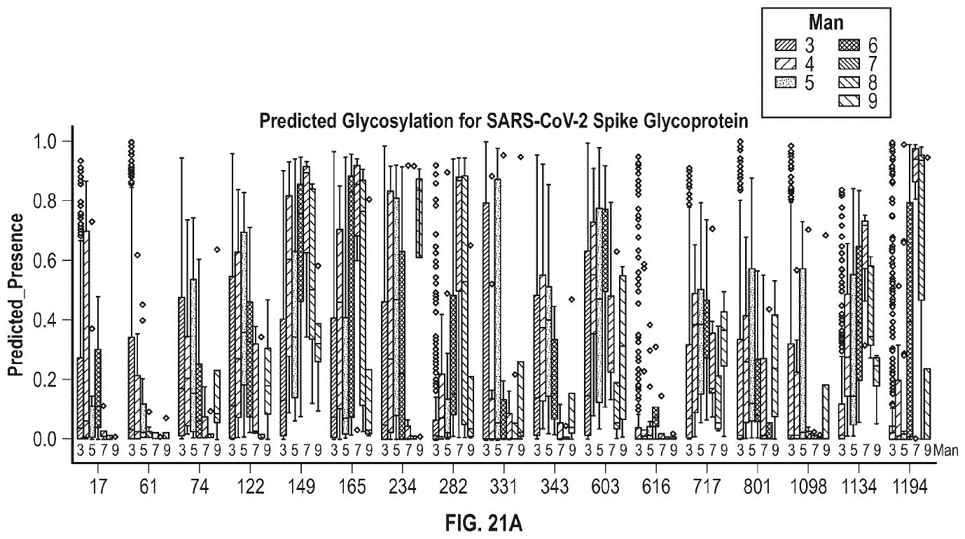

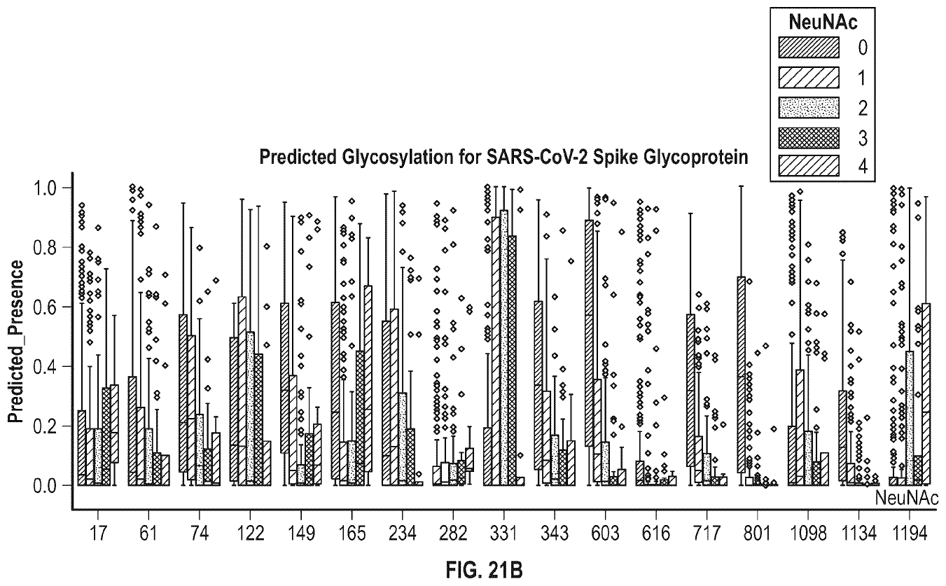

The model could also capture variation in SARS-CoV-2 glycan complexity. Oligomannose at N234 is consistently high (80-100%) and appears necessary to support the open ACE2-binding spike conformation. Predictions made by models described herein (e.g., InSaNNe) show strong preference for Man5 and Man9 structures and a strong preference against sialylation (FIG. 20C). The next-most immature sites, N717 and N801 (30-55%), see a near-complete obliteration of predicted sialylation ((FIG. 20C). Predictions for all glycosylation sites were mostly consistent with empirical observations (FIG. 21). The spike of new strains was also examined to see if their glycosylation could be predicted.

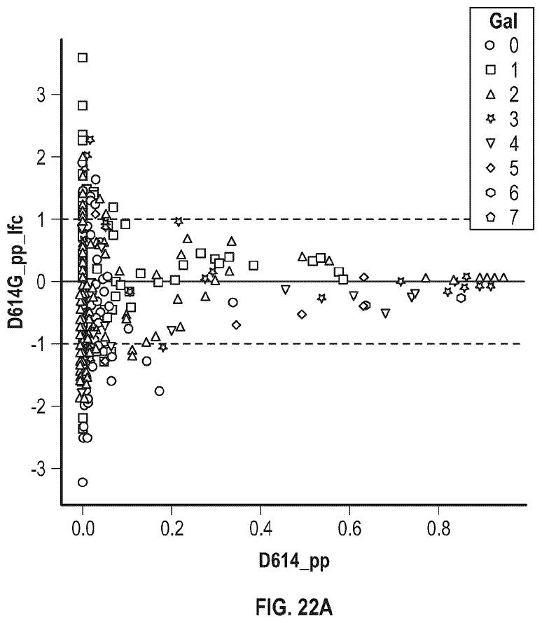

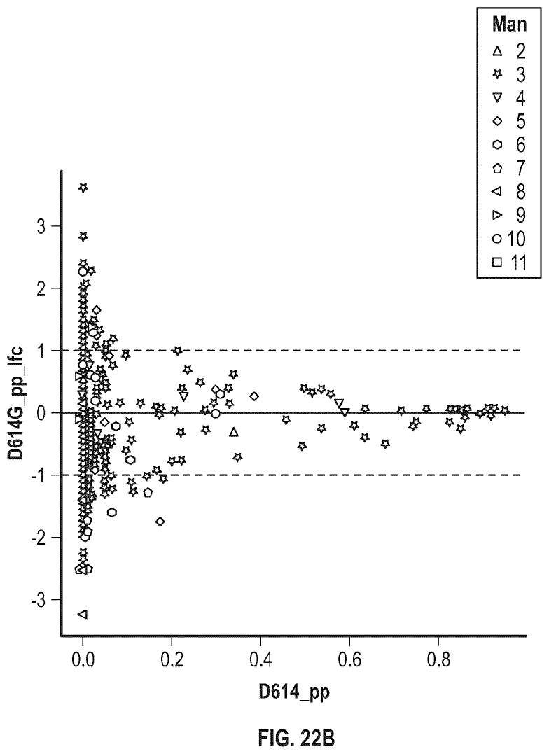

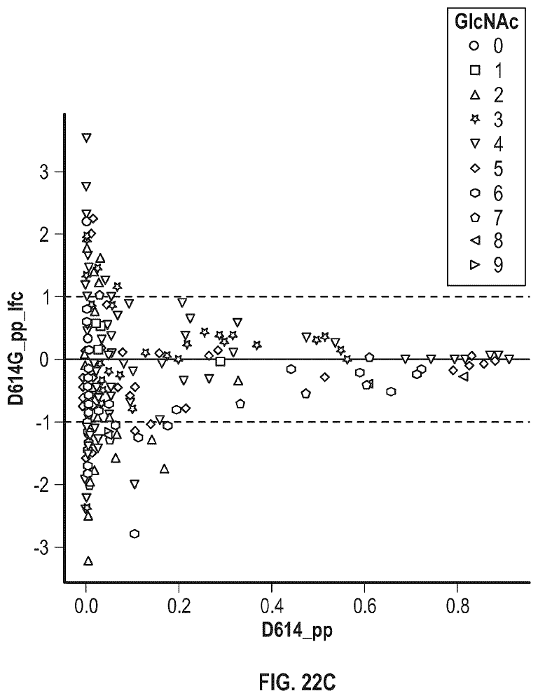

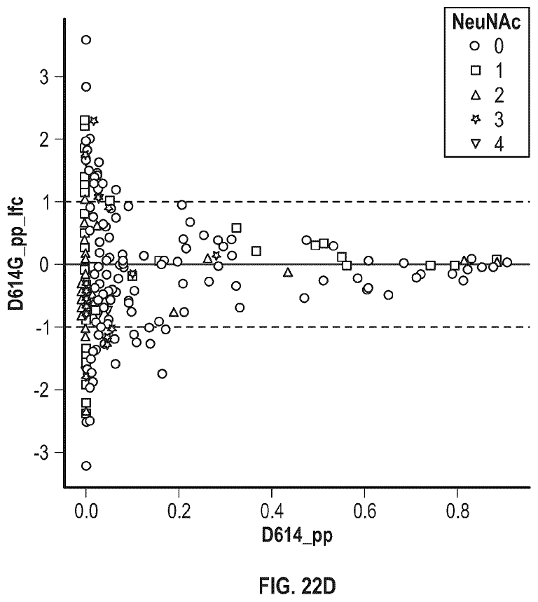

Examining site N616 in a simulated D616G variant (FIG. 22) and N717 in a T714I variant (FIG. 20D), distinct changes in predicted glycosylation were found. To focus attention on relevant changes, those with non-negligible wt predicted-presence (>0.1) and substantial fold change (|log FC|>1) relative to the wt were further examined. At site N717 in the T714I variant, many asialylated sugars with between 1 and 3 galactose decreased relative to wt. Additionally, a small number of sugars with 0-2 sialic acids and 1-4 galactose increased. Though InSaNNe predicts site N717 becomes variably hospitable to mono, di, tri and tetra-antennary sialylated and asialylated structures, empirically, it is an oligomannose site suggesting these terminal galactoses may not be visible without additional mutations to the site. Distinctly, the InSaNNe reveals few confident changes at site N616 in the D614G variant (FIG. 22)

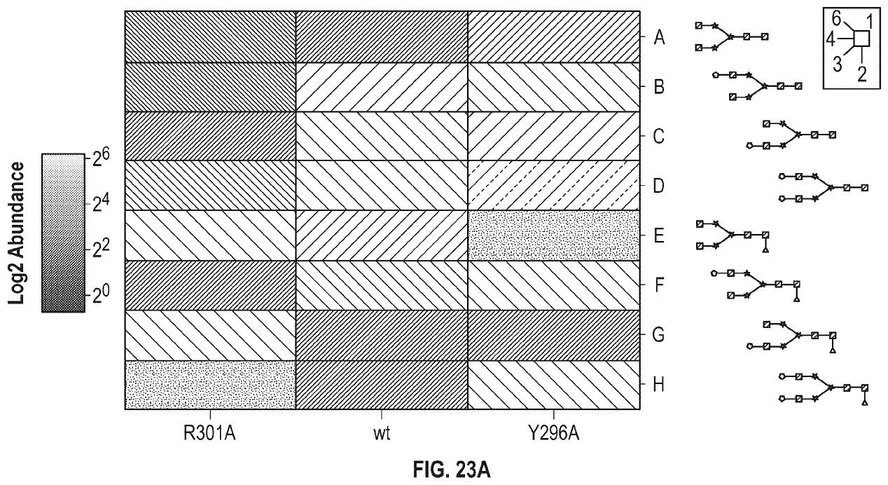

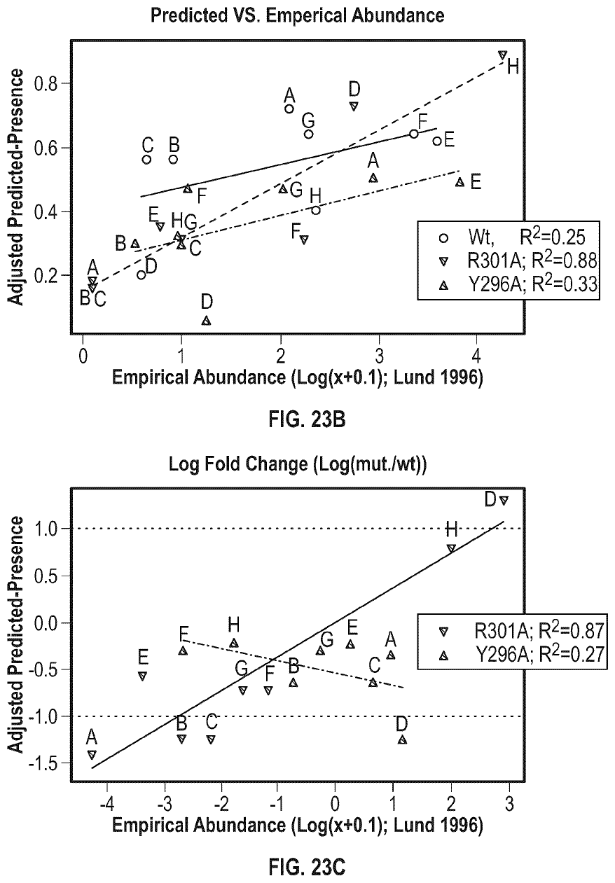

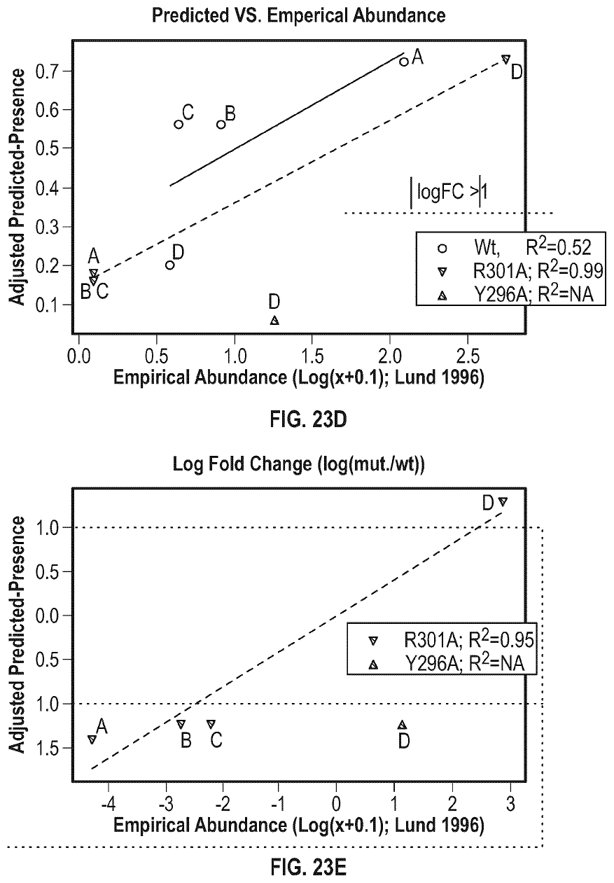

Mutations resulting in differential glycosylation in IgG3 have been reported. These data measured the abundance of 8 complex biantennary structures in human IgG3 for w/ and glycosite (N297; P01860.N227) proximal mutants. The wt IgG3 shows a preference for core-fucose and b1-6-branch galactose, R301A increased all terminal galactose, and Y296A accepted no galactosylation (FIG. 23A). As they found, primary protein structure can profoundly influence glycosylation.

InSaNNe predictions for the R301A and Y296A mutants were compared and background-adjusted predicted-presence and change in adjusted predicted-presence were found to correlate with empirical occupancy. Abundance-prediction was high for the R301A abundance (R²=0.876; FIG. 23B) and moderately predictive of wt abundance (R²=0.25; FIG. 23B). Predicted-presence was a moderate predictor of measured IgG3:N297 in the Y296A mutant (R²=0.33; FIG. 23B). Prediction accuracy increased when changes in predicted and observed values were compared. The log fold-change in predicted-presence in R301A relative to wt was highly correlated with measured log-abundance (R²=0.87; FIG. 23C). Yet, the consistency in predicted vs observed change for Y296A decreased dramatically (R<0, R²=0.27; FIG. 23C). To further probe the failure to predict change in Y296A, glycans with a predicted absolute log fold-change less than 1 were removed, and it was found that abundance prediction accuracy for wt (R²=0.52), R301A (R²=0.99), and log fold-change (R²=0.95) increased (FIG. 23D-23E) while nearly all predictions for Y296A dropped out. These results suggest that InSaNNe can predict changes in abundance, not only presence.

### Methods

### Site-Specific Glycosylation Training Set Construction

As described elsewhere herein, empirical site-specific glycosylation data from humans was obtained from UnicarbKB and Glyconnect with supplemental information from GlyGen. The protein structure annotation was done using the Structural Systems Biology (SSBio) package in python. Protein structure analysis was performed in Python v2.7.15 using SSBIO v0.9.9.8 to retrieve and calculate existing empirical and homology models from PDB and SWISSMOD (PDBe SIFTS), de novo homology models (I-TASSER v5.1), sequence properties (EMBOS v6.6.0.0 pepstats), sequence alignment (EMBOS v6.6.0.0 needle), secondary structure (DSSP v3.0.0, SCRATCHv1.1::SSpro and SCRATCHv1.1::SSpro8), solvent accessibility (DSSPv3.0.0 and FreeSASAv2.0.2), and residue depth (MSMSv2.2.6.1). Additional amino acid aggregate features were calculated using R::seqinr. Glycan structures were annotated using a combination of glypy and GlyCompare for structure parsing and comparison respectively. All glycan substructures, a connected subset of monosaccharides with and without linkage information, were extracted from each glycan, merged to make a superset of substructures, then mapped to each glycan, thus resulting in a mapping from every glycan in the input database to shared substructures.

For the dataset used to train SweetNet, 2,313 unique glycosylation events were extracted from UniCarb. This included the glycan sequence that was observed and the sequon (14 amino acids, with the glycosylated amino acid in the center) and structural information in the form of additional amino acids within 6 Å if structural simulations converged. As negative examples, the same number of combinations of sequons and glycans that have not been observed was generated.

### Model Construction

All glycan-sequon matching models comprised (1) a recurrent neural network that analyzed the amino acid sequence of the sequon, (2) another recurrent neural network analyzing the amino acids of the three-dimensional sequon surroundings, (3) a model analyzing the glycan sequence, described below, and (4) a part consisting of fully connected layers to use the concatenated features generated by the previous modules to predict whether a glycan is permissible on a sequon. The recurrent neural networks consisted of a 128-dimensional embedding layer followed by two bidirectional long short-term memory (LSTM) layers. The fully connected model part consisted of a linear layer, a leaky ReLU (rectified linear unit) activation function, a batch normalization layer, and a multi-sample dropout scheme followed by a sigmoid function.

There different model architectures for the glycan analysis module were compared. For assessing GlyCompare, the glycan analysis module comprised a fully connected neural network using the 12,259 GlyCompare features as inputs for two linear layers interspersed with dropout, leaky ReLU, and batch normalization layers. For the model containing a SweetTalk-based language model for glycan analysis, glycans were converted to glycowords, and a bidirectional recurrent neural network was use. For the SweetNet-based model, glycans were converted to graphs by constructing a list of nodes (representing monosaccharides or linkages) and edges to denote graph connectivity. The corresponding model contained an embedding layer and three graph convolutional layers, interspersed by leaky ReLUs, Top-K pooling layers, and both global mean and global maximum pooling operations. Model architectures and hyperparameters were optimized using cross-validation.

### Model Training and Prediction

All models were trained with an NVIDIA® TeslaU K80 GPU using PyTorch. The data were split on a protein level into 80% for training and 20% for testing. For the RNNs, all protein and glycan sequences were brought to the same length by padding. Linear layers and RNNs were initialized using Xavier initialization while SweetNet-type models were initialized using a sparse initialization scheme with a sparsity of 10%.

A batch size of 32 was used for all models. As an optimizer, adaptive moment estimation (ADAM) was used with a weight decay value of 0.00001 and a starting learning rate of 0.00001, which decayed according to a cosine function over 170 epochs. Models were trained for a maximum of 250 epochs, with an early stopping criterion of 25 epochs without a decrease in validation loss. Binary cross-entropy was used as a loss function. Beginning from epoch 150, stochastic weight averaging was additionally employed with a learning rate of 0.0001.

Presence or absence of each glycan may be predicted from the trained InSaNNe model. To heuristically boost signal for glycans with limited representation in the training set, a naturalistic background of predicted presence was generated for each glycan. Predictions were generated from all training-set sequons to capture the biases and variation of the dataset as a background predicted-presence distribution for each glycan. The background-adjusted predicted-presence is the product of predicted presence and the predicted-presence cumulative probability (statsmodels::ECDF v0.12.2) relative to the naturalistic background for that glycan.

### Supplemental Results

Protein Structure Optimization and Ablation Demonstrates that all Included Feature Types Support Predictive Performance

To determine suitable dataset preparations, a random forest classifier was trained to distinguish oligomannose from complex glycosylation sites, given protein structure and surface data. Dataset preparations included protein structure model type (ab initio [I-TASSER], curated [SWISSMOD], or empirical [PDB]), and proximity radius defining “proximal” amino acids (4-10 Å). Hyperparameters were optimized using 500 iterations of grid-search. Labels were balanced using up-sampling and performance was evaluated using area under the receiver-operator curve (AUROC), sensitivity and specificity on two iterations of six-fold cross-validation; each fold contained non-overlapping groups of proteins to avoid overfitting due to protein identity.

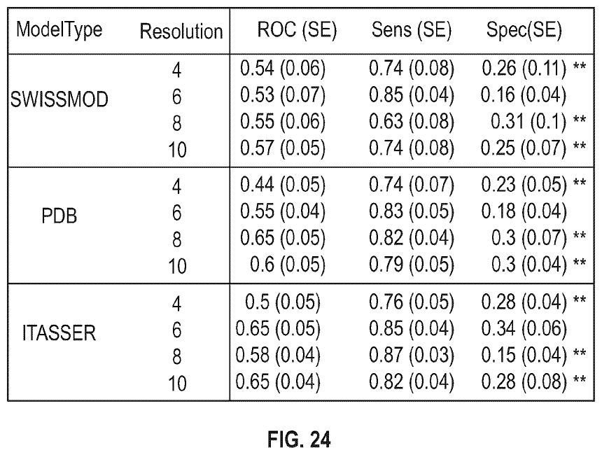

By varying these parameters, the optimal protein model and annotation resolution was evaluated (FIG. 24). Models trained on I-TASSER protein structures with a 6 Å-annotation resolution showed the high performance across all three metrics relative to random forest models trained on I-TASSER proteins annotated at other resolutions. Among models trained using PDB protein structures, those trained on data annotated at 8 Å performed well across all three metrics. The best PDB-trained models measured, on average, comparable AUROC, a 3.5% decline in sensitivity and 13% decline in specificity compared to I-TASSER-trained models. SWISSMOD-trained models did not have a clear best resolution though average scores were mostly comparable to those trained using PDB or I-TASSER structures.

Towards determining the importance of different protein structure annotations, an ablation analysis was performed by removing major types of data from the training set and comparing performance to models trained on all data (FIG. 25). The significance of each depletion in performance relative to models trained on all data was pooled (2-sample t-test, Fisher's method for pooling p-values); p-values were pooled within each ablation and performance metric across models trained on I-TASSER, PDB and SWISSMOD protein structures at all resolutions. AUROC, sensitivity and specificity are all sensitive to ablations in secondary structures, depth, and upstream amino acids (FDR<0.01475). Overall, each major data-type may maintain performance across all three metrics.

### Example 4: Trained Algorithm for Predicting Core Fucosylation

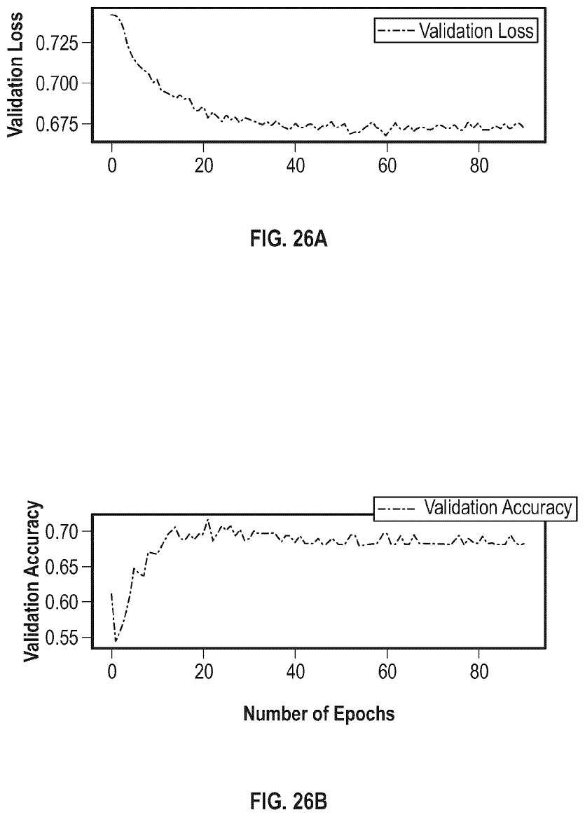

A trained algorithm as described elsewhere herein was trained on glycoprotein data to predict the likelihood of core fucosylation at a glycosite in a given protein sequence. Input glycoprotein data was split into a test set to train the network and a validation set for cross-validation of the trained algorithm. FIG. 26A shows the validation loss as a function of training epoch, indicating that the trained algorithm a generalizable model for predicting core fucosylation of previously unseen protein sequences. FIG. 26B shows the validation accuracy as a function of training epoch, demonstrating that the trained algorithm may correctly predict core fucosylation of previously unseen protein sequences.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of modifying a reference glycopeptide to alter a glycan substructure of a glycosite of the reference glycopeptide to produce a modified glycopeptide, the method comprising: calculating whether there is a positive or negative IMR association between one or more amino acid substitutions of a protein feature proximal to the glycosite and the glycan substructure, and generating the modified glycopeptide having the one or more amino acid substitutions if a magnitude of the IMR association is at least a threshold value.

2. The method of claim 1, wherein the threshold value is about 50%, 60%, 70%, 80%, 90%, or higher.

3. The method of claim 1, wherein the IMR is as generalized estimating equation (GEE) IMR.

4. The method of claim 1, wherein the IMR is a Fisher's exact test IMR.

5. The method of claim 3, wherein the IMR is significant if it has a false discovery rate (FDR) correction less than about 0.1.

6. The method of claim 3, wherein the IMR is significant if it has a p-value less than about 0.05.

7. The method of claim 1, wherein the IMR comprises a logarithm of an odds ratio (log OR) with a magnitude greater then about 1.

8. The method of claim 1, wherein the IMR comprises a log OR with a magnitude greater then about 0.5.

9. The method of claim 1, wherein the IMR comprises a log OR with a magnitude greater then about 0.1.

10. The method of claim 1, wherein the IMR association is determined using a matrix describing the expected glycoimpact of the one or more amino acid substitutions.

11. The method of claim 1, wherein the IMR association is determined at least based on the identity of one or more amino acids.

12. The method of claim 1, wherein the IMR association is determined at least based on the proximity of the one or more amino acids to the glycosite.

13. The method of claim 12, wherein the proximity is the distance from the glycosite as measured in angstroms.

14. The method of claim 13, wherein the proximity is less than or equal to about 6 angstroms to about 25 angstroms.

15. The method of claim 12, wherein the proximity is the number of amino acids between the each of the one or more amino acids and the glycosite.

16. The method of claim 15, wherein the distance is about 1, 2, 3, 4, 5, 6, 7, 8, 9, 10, 11, 12, 13, 14, or 15 amino acids.

17. A method of modifying a reference glycopeptide to alter a glycan substructure of a glycosite of the reference glycopeptide to produce a modified glycopeptide, the method comprising: substituting one or more amino acids of a protein feature proximal to the glycosite to generate the modified glycopeptide.

18. The method of claim 1, wherein the protein feature proximal to the glycosite comprises a structural feature.

19. The method of claim 18, wherein the structural feature is less than or equal to about 6 angstroms to about 25 angstroms from the glycosite.

20. The method of claim 18, wherein the structural feature is a secondary structure comprising a beta strand, alpha helix, extended strand, beta-bridge, turn, or bend, or a combination of two or more thereof.

21. The method of claim 1, wherein the protein feature proximal to the glycosite comprises an amino acid within about 6 amino acids of the glycosite in the N- or C-terminal direction.

22. The method of claim 1, wherein the glycan substructure is selected from Table 2 or Table 3.

23. The method of claim 1, employing a computational approach.

24. The method of claim 1, wherein the structure of the reference glycopeptide is or has been determined using X-ray crystallography, homology modeling, and/or de novo prediction based on primary amino acid sequence.

25. The method of claim 1, further comprising administering a therapeutically effective amount of the modified glycopeptide to a subject in need thereof based at least in part on the altered glycan substructure of the modified glycopeptide.

26. A modified glycopeptide having a first glycan substructure that is different from a reference glycan substructure of a glycosite of a reference glycoprotein, wherein the modified glycopeptide has one or more amino acid substitutions of a protein feature proximal to the glycosite as compared to the reference glycoprotein.

27. The modified glycopeptide of claim 26, wherein the protein feature proximal to the glycosite comprises a structural feature.

28. The modified glycopeptide of claim 27, wherein the structural feature is less than or equal to about 6 angstroms to about 15 angstroms from the glycosite.

29. The modified glycopeptide of claim 27, wherein the structural feature is a secondary structure comprising a beta strand, alpha helix, extended strand, beta-bridge, turn, or bend, or a combination of two or more thereof.

30. The modified glycopeptide of claim 26, wherein the protein feature proximal to the glycosite comprises an amino acid within about 6 amino acids of the glycosite in the N- or C-terminal direction.

31. The modified glycopeptide of claim 26, wherein the glycan substructure is selected from Table 2 or Table 3.

32. The modified glycopeptide of claim 26, wherein the protein feature is selected from Table 2 or Table 3.

33. A method comprising administering a therapeutically effective amount of the modified glycopeptide of claim 26, to a subject in need thereof based at least in part on the first glycan substructure of the modified glycopeptide.

34. A modified glycopeptide having an increase, decrease, or change in a glycan structure at a glycosite of the modified glycopeptide as compared to a reference glycopeptide, as determined based on the associations of Table 2 and/or Table 3 (e.g., wherein the modified glycoprotein has a Phe within 5 amino acids upstream of the glycosite, and the reference glycopeptide does not have a Phe within 5 amino acids upstream of the glycosite of the reference glycopeptide).

35. A method comprising administering a therapeutically effective amount of the modified glycopeptide of claim 34 to a subject in need thereof based at least in part on the increase, decrease, or change any of the glycan features selected from Table 1.

36. A method for determining the effect of a variation of a reference sequence on glycosylation of a first glycosite in the reference sequence, wherein the reference sequence comprises the first glycosite and a second glycosite, the method comprising:
(a) providing a plurality of sequences comprising (1) the reference sequence and (2) a plurality of variant sequences each having a different glycosylation feature at the second glycosite as compared to the reference sequence; and
(b) for each of the plurality of variant sequences: applying a trained algorithm to calculate the predicted presence of a glycosylation feature at the first glycosite based at least on the identity of the glycosylation feature at the second glycosite; thereby determining the effect of the variation of the reference sequence on glycosylation of the first glycosite.

37. A method for determining the effect of a variation of the structure of a reference sequence on glycosylation of a glycosite in the reference sequence, the method comprising:
(a) providing a plurality of sequences comprising (1) the reference sequence and (2) a plurality of variant sequences having one or more amino acid substitution as compared to the reference sequence; and
(b) for each of the plurality of variant sequences: applying a trained algorithm to calculate the predicted presence of a glycosylation feature at the glycosite of each variant sequence based at least on the structure of the variant sequence; thereby determining the effect of the variation of the reference sequence structure on glycosylation of the glycosite.

38. The method of claim 37, wherein the structure is secondary structure, tertiary structure, or quaternary structure, or a combination of two or more thereof.

39. The method of system of claim 1, wherein the sequence is a viral sequence.

40. A method for determining the likelihood that one or more glycans from a plurality of candidate glycans will be found at a glycosite of a viral sequence, the method comprising:
(a) providing the viral sequence and the plurality of candidate glycans, observed glycans, desired glycans, undesired glycans;
(b) for each of the plurality of candidate, observed, desired, or undesired glycans at each glycosite: applying a trained algorithm to calculate a predicted presence for each glycan at the glycosite of the sequence; and
(c) computer processing the predicted presence for each of the plurality of candidate, observed, desired, or undesired glycans to determine the likelihood that the one or more glycans will be found at the glycosite of the sequence.

41. A method of determining a likelihood of a disease or disorder associated with a glycoprotein in an individual, the method comprising:
calculating a first IMR association between a glycosite of the glycoprotein and a glycosylation feature;
calculating a second IMR association between the glycosylation feature and a glycosite of a modified glycoprotein, wherein the modified glycoprotein comprises one or more amino acid substitutions relative to the glycoprotein; and
determining said likelihood based on a difference between said first IMR and said second IMR.

42. A method for determining an IMR association between a glycosylation feature and one or more candidate glycoconjugates, the method comprising:
(a) applying a trained algorithm to one or more candidate glycans to calculate a predicted presence of the glycosylation feature at a glycosite of at least a subset of the one or more candidate glycoconjugates; and
(b) estimating a likelihood of the glycosylation feature at the glycosite of the at least a subset of the one or more candidate glycoconjugates.

43. The method of claim 42, further comprising: synthesizing a glycoconjugate if the likelihood is above a threshold.

44. The method of claim 42, further comprising predicting a pathogenicity of a mutation based on the likelihood calculated in (b).

45. The method of claim 42, comprising administering to an individual a gene therapy vector based on said likelihood calculated in (b).

46. The method of claim 42, wherein the at least a subset of the one or more glycoconjugates comprises a protein, peptide, polynucleotide, lipid, sugar, small molecule, or part thereof.

47. The method of claim 42 wherein the at least a subset of the one or more glycoconjugates comprises a surface protein of a cell.

48. A method for determining the importance of a glycosite, comprising
(a) providing, in computer memory, one or more datasets comprising co-evolution or conservation data associated with the glycosite;
(b) identifying one or more features of the glycosite; and
(c) calculating, with at least one computer processor, an importance of the glycosite based at least in part on the one or more datasets and the one or more features.


SEQUENCE LISTING (67 sequences)

SEQ ID NO: 1 - protein, 63 aa, Papio anubis
NPADAVQRILGGHLDAKGSFPWQAKMVSRHNLTTGATLINEQWLLTTAKNLFLNHSENATAKD

SEQ ID NO: 2 - protein, 63 aa, Papio anubis
QYNCLGCVHPISTQSPELEPILRHGVQYFNNNTQHSSLFTLSEVKRAQRQVVAGWNFLITYSI

SEQ ID NO: 3 - protein, 63 aa, Homo sapiens
NPANPVQRILGGHLDAKGSFPWQAKMVSHHNLTTGATLINEQWLLTTAKNLFLNHSENATAKD

SEQ ID NO: 4 - protein, 63 aa, Homo sapiens
QYDCLGCVHPISTQSPDLEPILRHGIQYFNNNTQHSSLFMLNEVKRAQRQVVAGLNFRITYSI

SEQ ID NO: 5 - protein, 63 aa, Homo sapiens
EGVLFFQGDREWFWDLATGTMKERSWPAVGNCSSALRWLGRYYCFQGNQFLRFDPVRGEVPPR

SEQ ID NO: 6 - protein, 63 aa, Homo sapiens
KWFYIASAFRNEEYNKSVQEIQATFFYFTPNKTEDTIFLREYQTRQNQCFYNSSYLNVQRENG

SEQ ID NO: 7 - protein, 63 aa, Papio anubis
NATAKDIAPTLTLYVGKKQLVEIEKVVLHPNYSQVDIGLIKLKQKVPVNERVMPICLPSKDYA

SEQ ID NO: 8 - protein, 63 aa, Papio anubis
EGILFFQGDHKWFWDLATGTMKKRYWSAVGNCSSALRWLGRYYCFQGNQFLRFNPVTGEVPPR

SEQ ID NO: 9 - protein, 63 aa, Homo sapiens
NATAKDIAPTLTLYVGKKQLVEIEKVVLHPNYSQVDIGLIKLKQKVSVNERVMPICLPSKDYA

SEQ ID NO: 10 - protein, 63 aa, Chlorocebus sabaeus; identical to SEQ ID NO: 1

SEQ ID NO: 11 - protein, 63 aa, Chlorocebus sabaeus
NATAKDIAPTLTLYVGKKQLVEIEKVVLHPNYSHVDIGLIKLKQKVPINERVMPICLPSKDYA

SEQ ID NO: 12 - protein, 63 aa, Chlorocebus sabaeus
EGILFFQGDHKWFWDLATGTMKERYWSAVGNCSSALRWLGRYYCFQGNQFLRFNPITGEVPPR

SEQ ID NO: 13 - protein, 63 aa, Chlorocebus sabaeus
QYNCLGCVHPISTQSPDLEPILRHGVQYFNNNTQHSSLFTLSEVKRAQRQVVAGWNFLITYSI

SEQ ID NO: 14 - protein, 63 aa, Chlorocebus sabaeus
SGEAPDSIIWKPDDAADLDATENPFDLLDFNQTQPERGDNNVIRIVGGRECKNGECPWQALLI

SEQ ID NO: 15 - protein, 63 aa, Macaca mulatta; identical to SEQ ID NO: 7

SEQ ID NO: 16 - protein, 63 aa, Macaca mulatta; identical to SEQ ID NO: 1

SEQ ID NO: 17 - protein, 63 aa, Macaca mulatta
NATVKDIAPTLTLYVGKKQLVEIEKVVLHPNYSQVDIGLIKLKQKVPVNERVMPICLPSKDYA

SEQ ID NO: 18 - protein, 63 aa, Macaca mulatta
NPADAGQRILGGHLDAKGSFPWQAKMVSRHNLTTGATLINEQWLLTTAKNLFLIHSENATVKD

SEQ ID NO: 19 - protein, 63 aa, Macaca mulatta
QYNCLGCVHPISTQSPDLEPILRHGVQYFNNKTQHSSLFTLSEVKRAQRQVVAGWNFLITYSI

SEQ ID NO: 20 - protein, 63 aa, Homo sapiens
QYDCLGCVHPISTQSPDLEPILRHGIQYFNNNTQHSSLFTLNEVKRAQRQVVAGLNFRITYSI

SEQ ID NO: 21 - protein, 63 aa, Macaca mulatta
QYNCLGCVHPISTQSPDLEPILRHGVQYFNNKTQHSSLFTLSEVKRAQRQDTGECTDNAYVDT

SEQ ID NO: 22 - protein, 63 aa, Macaca mulatta
SGEAPDNIIWKPDDAADLDATENPFDLLDFNQTQPERGDNNLIRIVGGRECENGECPWQALLI

SEQ ID NO: 23 - protein, 63 aa, Callithrix jacchus
NPANPVQRILGGHLDAKGSFPWQAKMVSRHNLTTGATLINEQWLLTTAKNLFLNHSENATAKD

SEQ ID NO: 24 - protein, 63 aa, Macaca mulatta; identical to SEQ ID NO: 8

SEQ ID NO: 25 - protein, 63 aa, Callithrix jacchus
EGVLFFQGDSEWFWDLTTGNIKKRSWPAVGNCSSALRWLGRYYCFQGNKFLRFNPATGEVPPR

SEQ ID NO: 26 - protein, 63 aa, Nomascus leucogenys
SGEGPDSITWKPYDAADLDPTENPFDLLDFNQTQPETGDNNLVRIVGGRECKDGECPWQALLI

SEQ ID NO: 27 - protein, 63 aa, Nomascus leucogenys
QYDCLGCVHPVSTQSPDLEPILRHGVQYFNNNTQHSSLFTLHEVKRAQRQVVAGLNFRITYSI

SEQ ID NO: 28 - protein, 63 aa, Nomascus leucogenys; identical to SEQ ID NO: 5

SEQ ID NO: 29 - protein, 63 aa, Pan troglodytes
QYDCLGCVHPISTQSPDLEPVLRHGIQYFNNNTQHSSLFTLNEVKRAQRQVVAGLNFRITYSI

SEQ ID NO: 30 - protein, 63 aa, Gorilla gorilla
GVLFFQEGDREWFWDLATGTMKERSWPAVGNCSSALRWLGRYYCFQGNQFLRFDPVRGEVPPR

SEQ ID NO: 31 - protein, 63 aa, Gorilla gorilla; identical to SEQ ID NO: 29

SEQ ID NO: 32 - protein, 63 aa, Gorilla gorilla
KPYDATDLDPTENPFDLLDLNQTQPERGDNNLTRIVGGQECKDGECPWQALLINEENEGFCGG

SEQ ID NO: 33 - protein, 63 aa, Gorilla gorilla
SGEAPDSITWKPYDATDLDPTENPFDLLDLNQTQPERGDNNLTRIVGGQECKDGECPWQALLI

SEQ ID NO: 34 - protein, 63 aa, Gorilla gorilla
NPANPVQRILGGHLDAKGSFPWQAKMVSRHNLTTGASLINEQWLLTTAKNLFLSHSENAIAKD

SEQ ID NO: 35 - protein, 63 aa, Gorilla gorilla; identical to SEQ ID NO: 23

SEQ ID NO: 36 - protein, 63 aa, Gorilla gorilla
NATAKDIAPTLTLYVGKKQLVEIEKVVLHPNYSQVDIGLIKLKQKVSVNERVMPICLPSKNYA

SEQ ID NO: 37 - protein, 63 aa, Macaca mulatta
NPADAVQRILGGHLDAKGSFPWQAKMVSRHNLTTGATLINEQWLLTTANNLFLNHSENATAKD

SEQ ID NO: 38 - protein, 63 aa, Macaca fascicularis
QYNCLGCVHPISTQSPDLEPILRHGVQYFNNNTQRSSLFTLSEVKRAQRQVVAGWNFLITYSI

SEQ ID NO: 39 - protein, 63 aa, Macaca fascicularis; identical to SEQ ID NO: 8

SEQ ID NO: 40 - protein, 63 aa, Macaca fascicularis; identical to SEQ ID NO: 1

SEQ ID NO: 41 - protein, 63 aa, Macaca fascicularis; identical to SEQ ID NO: 7

SEQ ID NO: 42 - protein, 63 aa, Pongo abelii
SREAPNNITWKPYDAADLDPTENPFDLLDFNQTQPERGDNNLVRIVGGRECKDGECPWQALLI

SEQ ID NO: 43 - protein, 63 aa, Pongo abelii
QYDCLGCVHPVSTQSPDLEPILRHGIQYFNNNTQHSSLFTLNEVKRAQRQVVAGLNFQITYSI

SEQ ID NO: 44 - protein, 63 aa, Pan troglodytes; identical to SEQ ID NO: 5

SEQ ID NO: 45 - protein, 63 aa, Pan troglodytes
SGEAPDSITWKPYDAADLDPTENPFDLLDFNQTQPERGDNNLTRIVGGQECKDGECPWQALLI

SEQ ID NO: 46 - protein, 63 aa, Pan troglodytes
KPYDAADLDPTENPFDLLDFNQTQPERGDNNLTRIVGGQECKDGECPWQALLINEENEGFCGG

SEQ ID NO: 47 - protein, 63 aa, Pan troglodytes
KWFYIASAFRNEEYNKSVQEIQATFFYFTPNKTEDTIFLREYQTRQDQCFYNTTYLNVQRENG

SEQ ID NO: 48 - protein, 63 aa, Pan troglodytes
NATAKDIAPTLTLYVGKKQLVEIEKVVLHPNYSQVDIGLIKLKQKVSVNERVMPICIPSKDYA

SEQ ID NO: 49 - protein, 63 aa, Pan troglodytes; identical to SEQ ID NO: 23

SEQ ID NO: 50 - protein, 63 aa, Ateles geoffroyi
NATAKDIAPTLTLYVCKNQLVEIEKVVLYPNYSQVDICLIKLKDKVPVNERVMPICLPSKDYA

SEQ ID NO: 51 - protein, 63 aa, Ateles geoffroyi; identical to SEQ ID NO: 23

SEQ ID NO: 52 - protein, 63 aa, Pan troglodytes; identical to SEQ ID NO: 9

SEQ ID NO: 53 - protein, 63 aa, Pan troglodytes
NPANPVQRILGGHLDAIGSFPWQAKMVSRHNLTTGATLINEQWLLTTAKNLFLNHSENATAKD

SEQ ID NO: 54 - protein, 63 aa, Homo sapiens; identical to SEQ ID NO: 46

SEQ ID NO: 55 - protein, 63 aa, Homo sapiens; identical to SEQ ID NO: 45

SEQ ID NO: 56 - protein, 63 aa, Pongo abelii; identical to SEQ ID NO: 5

SEQ ID NO: 57 - protein, 63 aa, Pongo abelii; identical to SEQ ID NO: 23

SEQ ID NO: 58 - protein, 62 aa, Pongo abelii
NATAKDIAPTLTLYVGKKQLVEIEKVVLHPNYSQVDIGLIKLKQKPVNERVMPICLPSKDYA

SEQ ID NO: 59 - protein, 63 aa, Papio hamadryas
NATAKDIAPTLTLYVGKKQLVEIEKVVLYPNYSQIDIGLIKLKQKVPVNERVMPICLPSKDYA

SEQ ID NO: 60 - protein, 63 aa, Papio hamadryas; identical to SEQ ID NO: 1

SEQ ID NO: 61 - protein, 63 aa, Papio hamadryas; identical to SEQ ID NO: 7

SEQ ID NO: 62 - protein, 14 aa, Homo sapiens
GTVLTRNETHATYS

SEQ ID NO: 63 - protein, 10 aa, Human immunodeficiency virus 1
PVQINCTRPN

SEQ ID NO: 64 - protein, 21 aa, synthetic construct
PWQAKVVSRHNLTQGATLLNE

SEQ ID NO: 65 - protein, 10 aa, synthetic construct
IHLENVTEEF

SEQ ID NO: 66 - protein, 10 aa, synthetic construct
LQCTNVTNNI

SEQ ID NO: 67 - protein, 10 aa, synthetic construct
NVTNNITDDM